(19) United States
(12) Patent Application Publication
(10) Pub. No.: US 2017/0039316 A1
(43) Pub. Date: Feb. 9, 2017

(54) Compositions, processes and algorithms for microbial detection

(71) Applicants: Yuriy Fofanov, Houston, TX (US); George E. Fox, Houston, TX (US); Richard Coale Willson, Houston, TX (US); B. Montgomery Pettitt, Houston, TX (US)

(72) Inventors: Yuriy Fofanov, Houston, TX (US); George E. Fox, Houston, TX (US); Richard Coale Willson, Houston, TX (US); B. Montgomery Pettitt, Houston, TX (US)

(21) Appl. No.: 10/973,113

(22) Filed: Oct. 25, 2004

Related U.S. Application Data
(60) Provisional application No. 60/519,417, filed on Nov. 12, 2003.
(60) Provisional application No. 60/532,210, filed on Dec. 23, 2003.

(51) Int. Cl. G06F 19/20; G06F 19/22; C12Q 1/68

(52) U.S. Cl. CPC G06F 19/20; C12Q 1/6888; C12Q 1/6837; C12Q 1/6874; G06F 19/22; C12Q 2600/16

(57) ABSTRACT

Processes for identifying whether any parasite or other organism is present in a host comprising: a. scanning for non-host signatures, b. scanning for one-error-removed non-host signatures; c. scanning for N-error removed non-host signatures; where N is selected to give the desired statistical certainty of the presence or absence of any parasite in the host. Algorithms useful for such detections and listings of specific signatures” (sequences or subsequences) for identifying specific microorganisms are also both provided.

### CROSS-REFERENCES TO RELATED APPLICATIONS

The present application claims priority of pending U.S. Ser. No. 60/519,417 filed Nov. 12, 2003 and U.S. Ser. No. 60/532,210 Filed Nov. 17, 2003 (Attorney Dockets 014APR/UH2003-48 and 014A′PR).

### STATEMENT REGARDING FEDERALLY SPONSORED RESEARCH OR DEVELOPMENT

No government funding has been received for this work to date.

### REFERENCE TO A SEQUENCE LISTING, A TABLE, OR A COMPUTER PROGRAM LISTING COMPACT DISC APPENDIX

Compact Discs 1-7, listing “Signatures” [sequences] are attached to, and made a part of, this Application. The contents of each CD are listed below, just before Table A. The text Table of Contents is identical with the CD contents. The CDs are enclosed as a part of the application under 37 Code of Federal Regulations Section 1.58.

### PROGRAM CODE APPENDIX

The computer programs and subroutines of the invention are set forth on the CD, which is enclosed as a part of the application under 37 Code of Federal Regulations Section 1.96.

### COPYRIGHT

Contained herein is material that is subject to international copyright protection. The copyright owner has no objection to the facsimile reproduction of the patent disclosure by any person as it appears in the Patent and Trademark Office patent files or records, but otherwise reserves all rights to the copyright whatsoever.

### BACKGROUND OF THE INVENTION

The detection and identification of microbes in complex samples from the environment, clinical specimens, food, etc. is an important and challenging problem. Naturally causative agents, including those responsible for malaria¹, infectious and food-borne bacterial diarrhea², and dengue fever³. The causative agents of such illnesses are extremely diverse in that they include bacteria, eukaryotic organisms and viruses⁴. Moreover, a considerable number of new/modified agents appear every year (SARS⁵, West Nile⁶, Monkeypox⁷, just to name a few). The threat of deliberate biological terrorism is also of concern⁸.

In addition to detecting known organisms, there is enormous value in the identification of previously unknown microorganisms responsible for environmentally and clinically important processes. The common process of identifying previously unknown human pathogens (microbes, viruses or their variants/modifications), for example, is uncertain and time consuming⁴. Often there are many weeks, months, or even years between the observation of initial infection and final identification of the causative agent. In addition to that, there are many microbes and viruses present in the human body, which do not represent an immediate danger to its physical condition, but may cause serious long-term consequences. Helicobacter pylori⁹, found to be the main cause of stomach ulcers, and papillomavirus¹⁰, many types of which cause benign skin tumors (warts) in their natural hosts, are examples.

Recent progress in genomics has led to new molecular approaches. Identification of bacteria, viruses, and rickettsia is increasingly based on the use of nucleic acid technologies⁴. In fact, the most detailed way to characterize a microbe or virus is sequencing and assembly of its complete genome. Several hundred complete genomes have become recently available and many more sequencing projects are in progress. Knowledge of complete genome sequences makes it possible to find unique “genetic signatures” of organisms. These signatures are employed with such approaches as polymerase chain reaction (PCR)¹¹ and microarrays of cDNA¹² and oligonucleotides¹³ for rapid determination of the presence of known organisms.

However, it is important to stress that many identification techniques require extraction of DNA of the organism of interest from complex samples (natural waters, rumen contents, food, blood, tissue, etc.), for analysis^{14,15}. This is not a trivial task, especially for viruses. For nucleic acid based technologies, the discrimination of a single organism/pathogen's DNA in the presence of background (host) DNA is also extremely difficult, because the concentration of other DNA is usually orders of magnitude higher than that of the organism/pathogen. A possible solution to this problem is to use PCR primers recognizing sequences which are uniquely present in the organism of interest and absent in other genomes¹⁶⁻¹⁸. In this case, PCR will exponentially amplify and effectively purify DNA of the organism of interest but not the other DNA, which may be present. It is also clear, that only nucleic acid sequences present in the organism of interest and absent in the background DNA can be used as potential specific identification signatures. This implies either extensive experimental effort (the current approach) or the extremely difficult computational task of finding such organism-unique and “background-blind” subsequences in the genome of each organism to be detected.

So far only a small fraction of microbial/viral genomes have been sequenced, however highly conserved genes, such as 16S ribosomal RNA (rRNA)¹⁸⁻²³, can be employed to distinguish a microbial species or group of species. The nucleotide sequence of 16S rRNA is regarded as the most useful marker for genetic characterization of bacteria. This molecule has been characterized in many thousands of bacteria as tabulated at the Ribosomal Database Project (RDP) site^{17-24,25} which now includes entries from over 72,600 aligned and annotated bacterial 16S rRNA sequences. These sequences can be used to construct a phylogenetic tree which positions each strain relative to every other. Organisms that are neighbors on this tree are genetically most similar and likely to share many biochemical properties

The straightforward sequence-based methods for pathogen discovery require partial sequencing and comparing results with known sequences present in existing databases GenBank, DDBJ, EMBL etc. Unfortunately such an approach cannot be used to identify new unknown organisms, and host/background DNA can cause considerable difficulty.

Another approach to identify known organisms is to find unique subsequences, present in this organism only (“signature” sequences) and use them as microarray probes or PCR primers. However, this requires not only knowledge of the sequence of the genome of interest, but also sequences of all other genomes which can be present in the sample. This method is restricted by the incompleteness of the list of sequenced genomes. Limitations of present technologies, such as melting temperature variations, possible hybridization with mismatches, primer-primer interactions, etc., make it also a nontrivial computational task. It becomes even more computationally difficult if the organism of interest has to be identified in the presence of much longer host DNA or in presence of numerous of other organisms (for example in environmental samples).

More than a thousand complete genomes, including hundreds of non-viral ones, have become available in the last several years. Many additional sequencing projects are in progress. The number of completed genomes, however, is so small compared to the number of extant species that true comparative genomics is still in its infancy. A relevant question thus arises as to whether there is sufficient material to look at from a statistical viewpoint²⁶.

### REFERENCES

1. Miller C L: Malaria in the third world. Del Med J 75:197; author reply 199-200, 2003
2. Stutman H R: Salmonella, Shigella, and Campylobacter: common bacterial causes of infectious diarrhea. Pediatr Ann 23:538-543, 1994
3. Lindback H, Lindback J, Tegnell A, et al: Dengue fever in travelers to the tropics, 1998 and 1999. Emerg Infect Dis 9:438-442, 2003
4. Relman D A: Detection and identification of previously unrecognized microbial pathogens. Emerg Infect Dis 4:382-389, 1998
5. Cooke F J, Shapiro D S: Global outbreak of severe acute respiratory syndrome (SARS). Int J Infect Dis 7:80-85, 2003
6. Bender K, Thompson F E, Jr.: West Nile virus: a growing challenge. Am J Nurs 103:32-39; quiz 40, 2003
7. Stephenson J: Monkeypox outbreak a reminder of emerging infections vulnerabilities. Jama 290:23-24, 2003
8. Cunha B A: Anthrax, tularemia, plague, ebola or smallpox as agents of bioterrorism: recognition in the emergency room. Clin Microbiol Infect 8:489-503, 2002
9. Hunt R H: Helicobacter pylori twenty years on. Can J Gastroenterol 17 Suppl B:7B, 2003
10. Mayeaux E J, Jr., Spigener S D: Treatment of human genital papillomavirus infections. Hosp Pract (Off Ed) 32:87-90, 1997
11. Markham A F: The polymerase chain reaction: a tool for molecular medicine. Bmj 306:441-446, 1993
12. Schena M, Shalon D, Davis R W, et al: Quantitative monitoring of gene expression patterns with a complementary DNA microarray. Science 270:467-470, 1995
13. Lipshutz R J, Fodor S P, Gingeras T R, et al: High density synthetic oligonucleotide arrays. Nat Genet 21:20-24, 1999
14. Diehn M, Relman D A: Comparing functional genomic datasets: lessons from DNA microarray analyses of host-pathogen interactions. Curr Opin Microbiol 4:95-101, 2001
15. Dozois C M, Daigle F, Curtiss R, 3rd: Identification of pathogen-specific and conserved genes expressed in vivo by an avian pathogenic Escherichia coli strain. Proc Natl Acad Sci USA 100:247-252, 2003
16. Relman D A, Loutit J S, Schmidt T M, et al: The agent of bacillary angiomatosis. An approach to the identification of uncultured pathogens. N Engl J Med 323:1573-1580, 1990
17. Maidak B L, Cole J R, Lilburn T G, et al: The RDP-II (Ribosomal Database Project). Nucleic Acids Res 29:173-174, 2001
18. Nichol S T, Spiropoulou C F, Morzunov S, et al: Genetic identification of a hantavirus associated with an outbreak of acute respiratory illness. Science 262:914-917, 1993
19. Woese C R, Fox G E, Zablen L, et al: Conservation of primary structure in 16S ribosomal RNA. Nature 254:83-86, 1975
20. Fox G E, Wisotzkey J D, Jurtshuk P, Jr.: How close is close: 16S rRNA sequence identity may not be sufficient to guarantee species identity. Int J Syst Bacteriol 42:166-170, 1992
21. Rossler D, Ludwig W, Schleifer K H, et al: Phylogenetic diversity in the genus Bacillus as seen by 16S rRNA sequencing studies. Syst Appl Microbiol 14:266-269, 1991
22. Relman D A, Schmidt T M, MacDermott R P, et al: Identification of the uncultured bacillus of Whipple's disease. N Engl J Med 327:293-301, 1992
23. Wilson K H, Blitchington R, Frothingham R, et al: Phylogeny of the Whipple's-disease-associated bacterium. Lancet 338:474-475, 1991
24. Ribosomal Database Project (RDP)
25. Cole J R, Chai B, Marsh T L, et al: The Ribosomal Database Project (RDP-II): previewing a new autoaligner that allows regular updates and the new prokaryotic taxonomy. Nucleic Acids Research 31:442-443, 2003
26. Fofanov Y, Luo Y, Katili C, et al: Short subsequences in genomes: How random are they? (submitted to Journal of Computational Biology), 2003

### BRIEF SUMMARY OF THE INVENTION

### Ultra Specific Probes and Primers for Genetic Testing

We have developed methods of identifying highly-specific PCR primers and DNA probes for species of interest in the presence of large backgrounds of other (e.g. human) DNA. This technology is useful in developing nucleic acid-based diagnostics, forensic assays, veterinary and agricultural assays. It also has utility in rapidly identifying unknown pathogens or biowarfare agents or other microorganisms.

### BRIEF DESCRIPTION OF THE DRAWINGS

FIG. 1 shows the frequency of presence of 9-15-mers in 114 microbial genomes. The solid line stands for the expected number of n-mers, M—genome length. All genomes fit in clearly pronounced pattern located near the random boundary.

FIG. 2 shows the frequency of presence of 5-10-mers in 541 RNA and 554 DNA viral genomes. The solid line stand for the expected number of n-mers, M—genome length. All genomes fit in clearly pronounced pattern located near the random boundary.

FIG. 3 shows an exemplary System configuration.

### DETAILED DESCRIPTION OF THE INVENTION

According to the invention, a process for identifying whether any parasite or other microorganism is present in a given host comprises: a. scanning for non-host signatures, b. scanning for one-error-removed non-host signatures; c. scanning for N-error removed non-host signatures; where N is selected to give the desired statistical certainty of the presence or absence of any parasite in the host. Specific identifiers (“signatures”) are provided herein for many pathogens and other microorganisms.

The straightforward sequence-based methods for pathogen discovery require partial sequencing and comparing results with known sequences present in existing databases GenBank, DDBJ, EMBL etc. Unfortunately such an approach cannot be used to identify new unknown organisms, and host/background DNA can cause considerable difficulty.

Another approach to identify known organisms is to find unique subsequences, present in this organism only (“signature” sequences) and use them as microarray probes or PCR primers. However, this requires not only knowledge of the sequence of the genome of interest, but also sequences of all other genomes which can be present in the sample. This method is restricted by the incompleteness of the list of sequenced genomes. Limitations of present technologies, such as melting temperature variations, possible hybridization with mismatches, primer-primer interactions, etc., make it also a nontrivial computational task. It becomes even more computationally difficult if the organism of interest has to be identified in the presence of much longer host DNA or in presence of numerous of other organisms (for example in environmental samples).

More than a thousand complete genomes, including hundreds of non-viral ones, have become available in the last several years. Many additional sequencing projects are in progress. The number of completed genomes, however, is so small compared to the number of extant species that true comparative genomics is still in its infancy. A relevant question thus arises as to whether there is sufficient material to look at from a statistical viewpoint³¹.

### More Detailed Disclosure

### Starting Materials

The starting material can be a “host” suspected to comprise microorganisms to be detected. Humans, rats, mice, and other organisms are suitable hosts.

### Processes

Using the “signatures”, techniques and algorithms provided herein, the presence of virtually any microorganism can be detected in the host.

### Apparatus

A high-speed computer is required for the large computational steps of applying the algorithms of the invention.

### Computational Methods and Algorithms

### 2.1 The Problem—

The shape of the frequency distribution for certain short subsequences: 2-4-mers [4-8] and 8-9-mers [9, 10] was proposed to be used to decide which microbial genome is being considered, based on a given random piece of genome or the entire genome. Algorithmically, such type of analyses employs a repeatable search for the short patterns in genomes, also known as the exact string matching problem.

Exact string matching is a well-developed area in computer science. The traditional definition of this problem is the following: Given a string P of size n called the pattern and the longer string T of size m called the text, the exact matching problem is to find all occurrences, if any, of pattern P in text T. Many algorithms have implemented this problem often integrating precomputation of either the pattern or the text.

Some algorithms, such as Rabin-Karp [13], Boyer-Moore [14], and Knuth-Morris-Pratt [15] apply precomputation to the pattern. The memory usage in such approaches is not very extensive and if n<<m; the time is proportional to the length of the text or sequence O(m). Other approaches are based on the idea of precomputing the text [16-18]. Such algorithms are more memory-expensive and the estimation of time required for precomputing is O(m), despite this string matching can be done extremely fast and depends only of the pattern size: O(n). Here it is necessary to note that there is some variation in the problem definition; in some cases one needs to find any occurrence instead of all occurrences of the pattern in the text, nonetheless the problem has the same notation in most of the literature.

To make an optimal choice as to which algorithm is better, one has to take into account additional parameters of the problem under consideration. In fact, we rarely match just one pattern against one sequence. In many cases we have a finite set of k sequences and a finite set of l patterns and need to perform kl searches to find the occurrence of all patterns in all sequences.

Another important parameter, which has to be considered, is the average ratio of the pattern length to the text size r=n/m. Let us provide a simple example. Assume we need to compare the Knuth-Morris-Pratt and the Suffix Tree algorithms. Taking into account that the precomputation time will be O(nl) for the Knuth-Morris-Pratt algorithm and O(km) for the Suffix Tree algorithm, we find the running time estimations are O(nl+lkm) for Knuth-Morris-Pratt and O(km+knl) for the Suffix Tree. Now, if usage of memory (which in some cases can be critical for Suffix Trees) is not a concern, we can easily estimate which algorithm would be preferable based on the parameters of a given real problem. In typical situations, the estimates above are expected for these two classes of algorithms. In general, one needs linear time for precomputing, which allows performing the search operation in linear time.

2.2 Calculation of the Presence of all Possible n-Mers in a Given Text

When applying this problem to the area of our interest, biology, four characteristics emerge:
(1) Relatively short pattern lengths: max(n)=25;
(2) Only 4 characters in the alphabet. All DNA sequences contain only 4 nucleotides A (adenine), T (thymine), C (cytosine), and G (guanine);
(3) For each value of n, the search has to be performed simultaneously for all possible 4ⁿ n-mers.
(4) Regarding each n-mer (pattern), we are interested only in its presence/absence in each genome (text), or in some rare cases how many times it appears in genome.

To take advantage of the specifics of our problem, in particular the fact that we can perform all calculations simultaneously for all n-mers for each given value of n, which is relatively small. We decided to employ an approach similar to the one used in the well-known counting-sort algorithm (for an example see [12]). The basic idea is to set in correspondence to each of the 4ⁿ n-mers a particular element in a counting array, A, and define the procedure to convert the n-mer character sequence to an index of an element in such an array. From this point of view, such an array can be treated as the extreme case of the hash table and the procedure to convert the sequence to the index can be treated as the hash function. Let us illustrate on a simple example. Assume we need to calculate how many different n-mers are present in the text T.

Algorithm 1.
NUMBER-OF-N-MERS-IN-TEXT(T,n)

1 for i ← 0 to 4ⁿ−1
2 do A[i] ← 0
3 sum ← 0
4 for j ← 0 to length [T]−n
5 do index=CONVERT_TO_INTEGER_VALUE(T(j,j+n−1))
6 if A[index]=0
7 then sum ← sum+1
8 A[index]=1
9 return sum

In this algorithm, T(j₁,j₂) stands for a substring of the string T, starting in position j₁ and ending in position j₂. The function CONVERT_TO_INTEGER_VALUE(s) is needed to convert string s of length n, which in our case is created using only a 4 character alphabet, to a unique integer value—corresponding to an index in the array A. In fact, if we assign to each character of our alphabet values from 0 to 3, each string can be interpreted as an integer value in a base-4 number system. Using a naïve algorithm this function can be implemented to have the run time O(n). We can utilize the fact that only 2 bits are needed for each character and that we read the text T sequentially, so that each string T(j₁,j₂) already contains n−1 elements of the next one T(j₁+1, j₂+1). Moreover we can implement the function CONVERT_TO_INTEGER_VALUE( ) using simple binary shift operations reducing execution time to O(1) time.

Thus the overall running time estimation for Algorithm 1 is O(4ⁿ+m). Practically, if both text T and the counting array A can be placed in memory it takes only seconds on a regular PC (1 GHz clock) to calculate, for example, how many 15-mers are present in the DNA sequence of Mycobacterium tuberculosis H37Rv (NC_000962, which has a length of 4,411,529 bp) genome as well as its complimentary sequence.

The biggest concern with this algorithm is memory usage, in particular the size of the counting array. The size of A can be defined based of the value of n and the characteristics of the problem. If we are interested only in the presence/absence of n-mers in a text, we need only 1 bit per element, where A [ . . . ]=1 indicates that the n-mer is present or A [ . . . ]=0 indicate its absence. In case of 17-mers, the necessary amount of memory required would be 4¹⁷=17,179,869,184 bits=2.15 Gb. A PC or workstation can meet such a requirement. It is, however, limiting for larger n-mers, say up to 20 in length.

If the required memory is not available or inconvenient, one can decompose the counting array A. The idea is a simple divide-and-conquer strategy: we divide our n-mer in to two parts, a prefix of size n₁ and a suffix of size n−n₁. One then creates the array A to track the appearance of (n−n₁)-mers in the suffixes and summarize results for all prefixes. Such an implementation is actualized in Algorithm 2.

Algorithm 2.
NUMBER-OF-N-MERS-IN-TEXT-2(T, n, n₁)

```
1 sum ← 0
2 for prefix ←0 to 4ⁿ₁ −1
3  do for i ← 0 to 4ⁿ⁻ⁿ₁ −1
4  do A[i] ← 0
5  for j ← 0 to length [T]−n
6  if (prefix=CONVERT_TO_INTEGER_VALUE
          (T(j,j+n₁−1))
7  then index=CONVERT_TO_INTEGER_VALUE
          (T(j+n₁,j+n−1))
8  if A[index]=0
9  then sum ← sum+1
10  A[index]=1
11 return sum
```

The operation count of Algorithm 2 is O(4ⁿ^{₁}(4ⁿ⁻ⁿ^{₁}+m))=O(4ⁿ+4ⁿ^{₁}m). In fact, in the case mentioned above, if the available computer has more than 2.5 Gb RAM, which means it can handle an array A necessary to keep track of 17-mers, it takes only about 4 times longer to count all 18-mers and 64 times longer to count all 20-mers. In practice, using Algorithm 2, the number of 20-mers in the Mycobacterium tuberculosis H37Rv genome can be calculated in less than one minute on a 1 GHz CPU. We were also able to perform such calculations for 20-mers in the human genome (available from NCBI) for all chromosomes in less than one hour. Note that in this algorithm increasing the prefix size n₁ caused an exponential increase of the run time. At the same time, because algorithm 2 can be easily parallelized (implemented in the for loop of line 2), the required run time can be essentially linearly decreased by increasing the number of processors. Such scaling makes the large-scale application of this algorithm very attractive.

For our problem area, another interesting task was to find how many n-mers are present in only one of two given texts T₁ and T₂, and how large is the number of n-mers that belong to both texts. There are at least three different ways to handle such a problem which were implemented and tested in our calculations:
(1) Run Algorithm 1 or Algorithm 2 three times to calculate the value of sum for text T₁ (sum₁), text T₂ (sum₂), and for both texts simultaneously (sum₁₂). Then sum₁-sum₁₂ will be the number of n-mers present only in T₁, sum₂-sum₁₂ will be the number of n-mers present only in T₂, and of course sum₁₂ will be the number of n-mers present in both texts;
(2) Use two arrays, A₁ and A₂, to keep track of the information regarding the presence/absence of n-mers in each text separately;
(3) Use 2 bits for each element of the array A and use them separately to track the presence/absence of n-mers in each text.
   2.3 Calculation of the Frequency of Presence of all Possible n-Mers.

Our approach, similar to that described in Algorithms 1 and 2 can also be used to calculate the actual number of n-mers present in the text. A rough or naïve algorithm can be created by employing the counting array A to store the number of appearances of each n-mer:

Algorithm 3.
FREQUENCY-OF-N-MERS-IN-TEXT(T,n)

1 for i ← 0 to 4ⁿ−1
2 do A[i] ← 0
3 for j ← 0 to length [T]−n
4 do index=CONVERT_TO_INTEGER_VALUE(T(j,j+n−1))
5 A[index]= A[index]+1
6 return A

As a result, Algorithm 3 in running time O(4ⁿ+m) produces array A, which in contrast to the cases discussed earlier contains integer numbers to store the presence in the text or sequence of each n-mer. The required memory in this case is much larger; instead of one bit we will need from 1 to 8 bytes to keep integer values. For example, using 4 bytes for each integer value the necessary memory for the case of 14-mers will be 1.07 Gb.

### Further Research

These algorithms are quite efficient but are extremely memory intensive. The problem is complicated when we need to store the n-mer calculations in a file, or if we want to do a comparative analysis of the presence/absence of n-mers in two or more than two sequences (the arrays of all the sequences need to be stored in memory). These algorithms ignore the fact that the n-mer array for n>11 is sparse and therefore can be compressed to save memory and hard disk space. Therefore a more careful look at the structure of the original data and the produced results is required.

### 3. Methods

### 3.1 Data Mining—

Before implementing our methods, we needed to gather a complete set of current data. The National Center for Biotechnology Information (NCBI) [25] is an excellent source for sequenced genomes. Complete genomic sequences of over 1000 viruses and over 100 microbes can be found in the NCBI database. The genomes represent both completely sequenced organisms and those for which sequencing is in progress. All three main domains of life—bacteria, archaea, and eukaryota—are represented, as well as many viruses and organelles.

With several newly sequenced genomes added frequently, the problem we first faced was, how to download the genomic sequence in the format that we need and keep our database up-to-date. FTP programs however cannot be used to download the sequences due to three problems. Firstly tracking down newly added sequences is a problem and downloading all the genomic sequences whenever a new sequence is added would be a waste of time. Secondly the NCBI database provides more information about the genomic sequences than we need. We want to download only the information pertinent to us and store it in a format which can be easily read by the analysis programs. Thirdly there are different sub-categories in the classification hierarchy (like viruses can be classified as single stranded DNA virus, double stranded DNA viruses, single stranded RNA viruses, etc). This information is very useful for our analysis but is not available in all FTP files; it is however available from the web site. To overcome these issues we developed a program in Java for data mining. It checks for the newly sequenced genomes and downloads the genomic sequences not present in our local database in our format.

The program can efficiently download all viral genomes (>1000) in 5-6 hrs on a normal computer with a fast Internet connection.

### 3.2 Algorithm

To make the previous algorithms more efficient it is necessary to take a more careful look at the structure of the original data and the produced results. In Tables 3.1 and 2 we show how many n-mers appear only once, more than once, and never appear in the genome of Mycobacterium tuberculosis H37Rv and in the human genome. As one can see, if the text size m is less than 4ⁿ, practically all n-mers are found to be present. However, for 4ⁿ>>m we face a very different situation: the majority of n-mers are simply absent in the text. Furthermore, the number of n-mers present in that text just once is much larger than the number of n-mers which are present more than once. It was already mentioned that a concern of our approach is the memory required for n>12. However, Tables 3.1 and 3.2 lead us to conclude that for such numbers the majority of array A will be occupied by zeros (sparse). In fact, the number of different n-mers cannot be larger than m−n. We also cannot expect the number of n-mers which appear more than once to be larger than (m−n)/2. Thus, to conveniently keep information about all present n-mers we need two arrays: one (R) to keep the “sequence” of n-mers and another (Q) to keep the integer number of its appearance. Of course it would be even easier to place both the n-mer and the number of its appearances in one data structure. The estimation of the memory usage is straightforward. For the array Q it is r_{integer}(m−n)/2≅r_{integer}m/2, where r_{integer} is the size reserved for the integer variable. For the array R it is rₙ(m−n)/2≅rₙm/2, where rₙ is the size reserved for the n-mer. Given the parameters of our problem area such a size is manageable. For example using 2 bytes for integers and 2 bits for all 16-mers in the sequence on the order of 3,000,000,000 (range of the human genome) we will need in the worst-case only 4.5 GB RAM. In practice, as seen from Tables 3.1 and 3.2 the number of n-mers present in real genomes is much less than hypothetical worst case. For larger genome like wheat or amphibians the above formulas provide a guide to calculate the memory required.

TABLE 3.1
The presence/absence statistics for Mycobacterium tuberculosis
H37Rv (NC_000962) genome of size 4,411,529 pairs
of nucleotides. Calculation was performed using both
(original and complementary) DNA sequences.

| | | | | Number of |
|---|---|---|---|---|
| | | | Number of | n-mers |
| | Number of | Number of | n-mers | present |
| n-mer | different | absent | present | more |
| size | n-mers 4ⁿ | n-mers | just once | than once |
| 7 | 16,384 | 0 | 2 | 16,382 |
| 8 | 65,536 | 152 | 318 | 65,066 |
| 9 | 262,144 | 9,234 | 11,826 | 241,084 |
| 10 | 1,048,576 | 186,370 | 147,776 | 714,430 |
| 11 | 4,194,304 | 1,918,866 | 862,428 | 1,413,011 |
| 12 | 16,777,216 | 12,415,707 | 2,624,671 | 1,736,838 |
| 13 | 67,108,864 | 60,778,599 | 4,938,895 | 1,391,370 |
| 14 | 268,435,456 | 260,837,795 | 6,777,575 | 820,086 |
| 15 | 1,073,741,824 | 1,065,526,021 | 7,798,705 | 417,098 |
| 16 | 4,294,967,296 | 4,286,498,557 | 8,245,983 | 222,756 |
| 17 | 17,179,869,184 | 17,171,299,773 | 8,424,979 | 144,432 |
| 18 | 68,719,476,736 | 68,710,863,745 | 8,499,559 | 113,432 |
| 19 | 274,877,906,944 | 274,869,272,195 | 8,534,649 | 100,100 |
| 20 | 1,099,511,627,776 | 1,099,502,979,652 | 8,555,193 | 92,931 |

TABLE 3.2
The presence/absence statistics for human genome of size 2,874,736,094
pairs of nucleotides. Calculation was performed using both
(original and complementary) DNA sequences.

| | | | | Number of |
|---|---|---|---|---|
| | | | Number of | n-mers |
| | Number of | Number of | n-mers | present |
| n-mer | different | absent | present | more |
| size | n-mers 4ⁿ | n-mers | just once | than once |
| 7 | 16,384 | 0 | 0 | 16,384 |
| 8 | 65,536 | 0 | 0 | 65,536 |
| 9 | 262,144 | 0 | 0 | 262,144 |
| 10 | 1,048,576 | 0 | 0 | 1,048,576 |
| 11 | 4,194,304 | 42 | 324 | 4,193,938 |
| 12 | 16,777,216 | 42,501 | 91,146 | 16,643,569 |
| 13 | 67,108,864 | 2,382,096 | 2,642,582 | 62,084,186 |
| 14 | 268,435,456 | 41,634,971 | 30,411,367 | 196,389,118 |
| 15 | 1,073,741,824 | 410,828,287 | 166,998,278 | 495,915,259 |
| 16 | 4,294,967,296 | 2,717,880,983 | 671,192,253 | 905,894,060 |
| 17 | 17,179,869,184 | 14,452,040,667 | 1,790,043,813 | 937,784,704 |
| 18 | 68,719,476,736 | 65,147,397,575 | 2,881,849,256 | 690,229,905 |
| 19 | 274,877,906,944 | 270,850,664,602 | 3,538,156,028 | 489,086,314 |
| 20 | 1,099,511,627,776 | 1,095,257,688,530 | 3,866,031,543 | 387,907,703 |

The following algorithm can be introduced to generate arrays Q and R:

Algorithm 4.
FREQUENCY-OF-N-MERS-IN-TEXT_2(T,n)

1 for i ← 0 to 4ⁿ−1
2 do A[i] ← 0
3 sum ← 0
4 for j ← 0 to length [T]−n
5 do index=CONVERT_TO_INTEGER_VALUE(T(j,j+n−1))
6 A[index]= A[index]+1
7 if A[index]=1
8 then sum ← SUM+1
9 A[index]=1
10 Use value of sum to reserve memory for arrays R and Q of size sum
11 counter ← 0
12 for i ← 0 to 4ⁿ−1
13 if A[i]=1
14 than R[counter]=i
15 Q[i]= A[i]
16 counter ← counter + 1
17 return Q,R

The Algorithm 4 produces two synchronized arrays Q and R of dynamically defined size sum. The total run time estimation of Algorithm 4 is O(4ⁿ+m+4ⁿ)=O(4ⁿ+m). Because the array R of present n-mers is created sorted, the time estimation to check the presence of any n-mer in such array requires logarithmic time O(log(sum)), so that the worst case would be O(ln(m)).

It is important to mention that because these two arrays (R and Q) represent the set of all n-mers present in the original sequence it is reasonable to place them in one data structure to store such data and use for future analysis. Traditional set operations such as union, intersection, and subtraction can be introduced, and implemented for or such objects with linear run time O(sum1+sum2), where sum1 and sum2 are sizes of arrays in two data structures under considerations.

### 3.3 Inclusion of Algebra Operations:

In the new RQ set, many useful functions have been integrated for enhanced analysis of the subsequences. The new functions include Union, Subtraction and Intersection among others. Each of these algebraic operations has a linear running time taking less than an hour on a standard PC.

Union—This function to calculate the union of the subsequences present in the all the microbial sequences provides us with the ability to analyze the presence and unanticipated absence of the subsequences within all the microbial genomes. It was observed that more than 8000 subsequences are not present in any of the microbial genomes.

These 8000 subsequences showed distinctive similarity between themselves which leads us to the conclusion that the absence of the subsequences are not random and further analysis needs to be done.

Union function.
R-Q-UNION (R1,R2,Q1,Q2)

| | |
|---|---|
| 1 | for i1← 0 to n1 and i2 ← 0 to n2 |
| 2 | if R1[i1] < R2[i2] and i1<n1 and i2<n2 |
| 3 | then UnionR[i] ← R1[i1] |
| 4 | UnionQ[i] ← Q1[i1] |
| 5 | i ← i +1 |
| 6 | i1 ← i1+1 |
| 7 | if R1[i1] > R2[i2] and i1<n1 and i2<n2 |
| 8 | then UnionR[i] ← R2[i2] |
| 9 | UnionQ[i] ← Q2[i2] |
| 10 | i ← i +1 |
| 11 | i2 ← i2+1 |
| 12 | if R1[i1] = R2[i2] and i1<n1 and i2<n2 |
| 13 | then UnionR[i] ← R1[i1]; |
| 14 | sum ← Q1[i1] + Q2[i2]; |
| 15 | if sum < 255 |
| 16 | then UnionQ[i] ← sum |
| 17 | else |
| 18 | UnionQ[i] ← 255; |
| 19 | i ← i +1 |
| 20 | i1 ← i1 +1 |
| 21 | i2 ← i2 +1 |
| 22 | if i1 = n1 |
| 23 | then UnionR[i] ← R2[i2] |
| 24 | UnionQ[i] ← Q2[i2] |
| 25 | i2 ← i2 + 1 |
| 26 | if i2=n2 |
| 27 | then UnionR[i] ← R1[i1] |
| 28 | UnionQ[i] ← Q1[i1] |
| 29 | i ← i +1 |
| 30 | i1 ← i1 +1 |

(R1, Q1) and (R2, Q2) are the RQ sets of two organisms on which the Union operation is to be performed and n1 and n2 are the number of different subsequences present in the two RQ sets, respectively. (UnionR, UnionQ) is the temporary RQ set used to store the results of the Union operations. In the RQ sets the sequences are ordered according to the unique index, which is calculated from the sequence. In the Union function both the sequences are traversed and the sequences that are present in either or both the RQ sets are stored in the UnionRQ set. Using the proposed algorithm and the RQ sets, the complete Union of the ten largest microbial genomes, as shown in Table 3.3, can be obtained in a relatively short period. The operation count of the Algorithm is (O (n1+n2)) where n1 and n2 are the length of the two RQ sets. For n-mer size less than 9, the calculations can be performed in a negligible amount of time and for higher n-mers it takes a reasonable amount of time on a standard PC as can be seen in table 1.

TABLE 1
The time required (in seconds) for calculating Union on the ten
largest microbial genomes. Calculation was performed using
both (original and complementary) DNA sequences.

| | 8- | 9- | 10- | 11- | 12- | 13- | 14- |
|---|---|---|---|---|---|---|---|
| Microbial Organisms | mers | mers | mers | mers | mers | mers | mers |
| Bradyrhizobium | 0 | 1 | 5 | 13 | 25 | 53 | 73 |
| japonicum strain | | | | | | | |
| Streptomyces | 0 | 0 | 3 | 10 | 27 | 45 | 64 |
| coelicolor | | | | | | | |
| Mesorhizobium loti | 1 | 1 | 3 | 10 | 26 | 45 | 67 |
| Nostoc sp. PCC | 0 | 1 | 3 | 10 | 26 | 45 | 65 |
| 7120. | | | | | | | |
| Pseudomonas | 0 | 1 | 3 | 11 | 26 | 45 | 63 |
| aeruginosa | | | | | | | |
| Pseudomonas | 0 | 0 | 3 | 11 | 26 | 44 | 65 |
| putida | | | | | | | |
| Xanthomonas | 0 | 1 | 3 | 10 | 26 | 45 | 64 |
| axonopodis | | | | | | | |
| Shewanella | 1 | 1 | 4 | 10 | 25 | 47 | 64 |
| oneidensis MR-1. | | | | | | | |
| Escherichia | 0 | 1 | 4 | 10 | 24 | 45 | 65 |
| coli K-12. | | | | | | | |
| Leptospira interrogans | 0 | 1 | 3 | 13 | 24 | 45 | 65 |
| serovar lai | | | | | | | |

Subtraction—The function to isolate the subsequence present in one particular genome and absent in all other genomes presents the excellent opportunity to find the subsequence unique to a particular organism and analyze its significance.

The function Subtraction incorporated in the R Q Set enables us to subtract subsequences present in one of the genomes with those present in other genomes.

Subtraction function.
R-Q SUBTRACTION (R1, R2, Q1, Q2)

1 for i1← 0 to n1
2 if R1[i1] = R2[i2] and i1<n1 and i2<n2
3 then i1 ← i1 +1
4 i2 ← i2 +1
5 if R1[i1] < R2[i2]
6 then SubtractionR[i] ← R1[i1]
7 SubtractionQ[i] ← Q1[i1]
8 i1 ← i1 + 1
9 i ← i + 1
10 if R1[i1] > R2[i2]
11 if i1 < y ->n
12 then i2 ← i2 +1
13 else
14 SubtractionR[i] ← R1[i1]
15 SubtractionQ[i] ← Q1[i1]
16 i1 ← i1 + 1
17 i ← i + 1
18 return

Just as with the Union function, in this algorithm (R1, Q1) and (R2, Q2) are the two RQ sets that are passed to the function as inputs and n1 and n2 are the number of different subsequences present in the two RQ sets, respectively. (SubtractionR, SubtractionQ) is the temporary RQ set used to store the result of (R2,Q2) being subtracted from (R1, Q1). This algorithm has a run time of O(n1) where n1 is the length of (R1, Q1) set. The operational count of the Subtraction algorithm is O(n1), where n1 is the number of different subsequences in the first organism. In the worst case the operational cost would be O(4ⁿ) where n is the length of the subsequence. In actuality the number of present subsequences is much less than the total possible subsequences for large subsequence lengths (>13).

The program when tested, once again, on the ten largest microbials for complete Subtraction for different subsequence sizes (8-15) gave the following result:

TABLE 3.4
The time required, in seconds, for complete subtraction of ten
largest microbial genomes. Calculation was performed using
both (original and complementary) DNA sequences.

| | 8- | 9- | 10- | 11- | 12- | 13- | 14- |
|---|---|---|---|---|---|---|---|
| Microbial Organisms | mers | mers | mers | mers | mers | mers | mers |
| Bradyrhizobium | 4 | 5 | 7 | 10 | 13 | 15 | 19 |
| japonicum | | | | | | | |
| Streptomyces | 5 | 4 | 7 | 8 | 10 | 13 | 17 |
| coelicolor | | | | | | | |
| Mesorhizobium loti | 3 | 5 | 7 | 8 | 10 | 13 | 15 |
| Nostoc sp. PCC 7120. | 4 | 3 | 6 | 8 | 10 | 12 | 17 |
| Pseudomonas | 3 | 4 | 5 | 6 | 7 | 10 | 13 |
| aeruginosa | | | | | | | |
| Pseudomonas putida | 4 | 4 | 5 | 7 | 10 | 11 | 14 |
| Xanthomonas | 2 | 2 | 5 | 5 | 6 | 7 | 11 |
| axonopodis | | | | | | | |
| Shewanella | 3 | 3 | 4 | 6 | 7 | 10 | 13 |
| oneidensis. | | | | | | | |
| Escherichia coli K-12. | 3 | 3 | 4 | 5 | 8 | 8 | 10 |
| Leptospira interrogans | 3 | 3 | 4 | 5 | 6 | 7 | 11 |
| serovar lai | | | | | | | |

Intersection—Another extremely useful function incorporated in the R Q set operations is the Intersection function. It is of great importance to analyze the subsequences which are common to two or to a set of genomes. The Intersection operation enables us to do exactly that.

The result of the Intersection operation performed on all of the available microbial genomes shows that the only subsequences that are universally present occur in the rRNA sequences. This result is in agreement with the fact that the ribosomal RNA genes are most conserved among all regions during the evolution in microbial genomes.

Intersection Function.
R-Q INTERSECTION (R1,R2,Q1,Q2)

1 for i1← 0 to n1 and i2 ← 0 to n2
2 if R1[i1] = R2[i2]
3 then IntersectionR[i] ← R1[i1]
4 if Q1[i1] > Q2[i2]
5 then IntersectionQ[i] ← Q1[i1]
6 else
7 IntersectionQ[i] ← Q2[i2]
8 i1 ← i1+1
9 i2 ← i2+1
10 if R1[i1] < R2[i2]
11 then i1 ← i1 +1
12 if R1[i1] > R2[i2])
13 then i2 ← i2 +1
14 return

Here (R1, Q1) and (R2, Q2) are the two RQ sets and n1 and n2 are the number of different subsequences present in the two RQ sets, respectively. (IntersectionR, IntersectionQ) is a temporary RQ set used to store the intersection result of (R1, Q1) and (R2, Q2). Like the other operations, this algorithm necessitates linear running time. Just like we did for the Union and Subtraction operations, we performed the Intersection operation on the ten largest microbial genomes. Running time for 8-mers was negligible (<1 sec) and the running time for 15-mers was less than a minute, as shown in Table 3.5, on a Standard PC having 1 Gb RAM.

TABLE 3.5
The time required, in seconds, for complete Intersection of ten largest
microbial genomes. Calculation was performed using both
(original and complementary) DNA sequences.

| | 8- | 9- | 10- | 11- | 12- | 13- | 14- |
|---|---|---|---|---|---|---|---|
| Microbial Organisms | mers | mers | mers | mers | mers | mers | mers |
| Bradyrhizobium | 1 | 2 | 5 | 13 | 22 | 32 | 38 |
| japonicum | | | | | | | |
| Streptomyces | 0 | 1 | 3 | 9 | 15 | 22 | 30 |
| coelicolor | | | | | | | |
| Mesorhizobium loti | 0 | 1 | 3 | 7 | 15 | 22 | 29 |
| Nostoc sp. PCC 7120 | 0 | 1 | 3 | 9 | 15 | 21 | 27 |
| Pseudomonas | 0 | 0 | 2 | 8 | 15 | 22 | 28 |
| aeruginosa | | | | | | | |
| Pseudomonas putida | 1 | 1 | 3 | 9 | 15 | 22 | 28 |
| Xanthomonas | 0 | 1 | 3 | 9 | 15 | 22 | 27 |
| axonopodis | | | | | | | |
| Shewanella | 0 | 1 | 4 | 7 | 16 | 22 | 28 |
| oneidensis. | | | | | | | |
| Escherichia coli K-12. | 0 | 0 | 3 | 8 | 16 | 21 | 32 |
| Leptospira interrogans | 0 | 1 | 3 | 8 | 14 | 22 | 28 |
| serovar lai | | | | | | | |

### 3.4 Implementation

For our analysis we downloaded all genomes currently available from the NCBI site [25] including microbial (107), viral (954), and multicellular organisms (5) genomes, with sizes ranging from 0.32 Kb (Cereal yellow dwarf virus-RPV satellite RNA NC_003533) to 2.87 Gb (human).

For our computations with multicellular organisms, microbial, and viruses, we used both complementary sequences for computational convenience because it is the way in which we can observe it based on the present technology (PCR, cDNA Microarrays, etc.). This trivially increases the amount of analyzed material by a factor of two. This apparent redundancy does not affect the statistical outcome and allows us to simplify the analysis.

In our study, we examined the presence/absence of short subsequences in more than one genome simultaneously. We also compared the frequencies of presence/absence of each n-mer in each of the genomes for 5≦n≦20. To our knowledge, no such studies have been done for n>11 due to the rapid growth of computational complexity with traditional algorithms. We performed such analyses separately in four different sets of genomes: RNA based viruses (542 genomes), DNA based viruses (412 genomes), Microorganisms (107 genomes) and Human. In each group the number of simultaneously present 5-18-mers was calculated for each pair of genomes. The human genome contains 24 chromosomes, for which the numbers of simultaneously present 7-20-mers were calculated for each pair of chromosomes.

To be able to perform calculations for longer (n>11) n-mers new algorithms and specific data structures (such as counting arrays [19] and incomplete search trees [20]) were utilized. The principal advantage of our approach is its time and memory efficiency, since only n-mers that are present in a genome under consideration (but not all possible 4ⁿ n-mers) are taken into account. This approach also provides an efficient way to store sequences for later use.

Microbial/Viral Fingerprints Using Random Subsets of n-Mers

One may use relatively small sets of randomly picked n-mers for differentiating between different viruses and organisms.

This idea can be illustrated by continuing our example for three microbial genomes. Let n* be the size of the n-mer, that fits the interval, where from 5% to 50% of all possible n-mers show up for a desirable range of genome lengths. In accordance with Table 4.5, we may choose the value n*=12. Let us randomly pick L 12-mers (say, L=1000). For example, L can be the number of probes placed on a microarray. Given a genome G₁ with the frequency of presence of n-mers p₁, we expect that K=p₁L n-mers present in G₁ will appear also in our random set, forming a “fingerprint” of G₁ (in our example, we expect 50<K<500). The probability, c, that the fingerprint of G₁ will exactly coincide with the fingerprint of some other genome G₂ (with the frequency of presence of n-mers p₂) is found in Appendix B. The result is

ε=(1−p₁−p₂+2p₁₂)^{L}. 5.3)

Here p₁₂ is the probability for the n-mer to be present in both genomes simultaneously. Let us consider the numeric example mentioned in Tables 4.3 and 4.4 of two species that are far from each other, Salmonella typhi vs. Mycobacterium tuberculosis H37Rv; p₁=0.3465, p₂=0.2600, p₁₂=0.1160; with L=1000 a remarkable accuracy of ε=1.7*10⁻²⁰⁴ can theoretically be achieved.

Given a desirable probability of error, e, one can determine the appropriate size, L, of a random set of n-mers which can be used for reliable identification of genomes as

$$L = \frac{log ɛ}{log ( 1 - {p}_{1} - {p}_{2} + 2 {p}_{12} )} . \qquad 5.4 )$$

For related organisms, the genomes may contain large common parts. This means that p₁₂ may be close to p₁ and p₂. To give a numeric example of close relatives, let us consider Staphylococcus aureus N315 vs. Staphylococcus aureus Mu50. Now p₁=0.198, p₂=0.203, p₁₂=0.197 and an accuracy of ε=10⁻¹⁰ can be achieved with L=4451. We would like to stress the logarithmic dependence of the sampling size or the number of probes on the microarray, L, on the error probability, ε. This feature is of principal importance for the estimation procedure under discussion.

Therefore, we can use practically any sufficiently random subset of n-mers of an appropriate size to construct a microarray to determine which organism a given DNA/RNA sample belongs to. Different sizes of n-mers must be employed for recognition of different organisms based on their genome lengths. Values of n that correspond to given intervals of genome lengths can be easily calculated using the above formulas. In fact, only 11 different n values, 7≦n≦17 can apply to a large variety of genome sizes from 1 Kb to 9 Gb.

The important advantage of such an approach is that it can be used without a priori knowledge of the sequence itself. This implies that there is no need to perform the expensive and time-consuming process of sequencing before array design. It is enough to obtain the purified DNA sample, hybridize it on a sufficiently random microarray chip and check which n-mers appear. Taking into account how accessible the DNA of thousands of microbial and viruses are, how easily each microarray can be produced, and the fact that we do not need to determine quantitative values of expression (we need just a yes/no answer)—it should be possible to produce an essentially universal microbial/viral DNA chip.

### Fingerprints of Closely Related Organisms

We next consider what happens when we try to compare closely related organisms using this approach (e.g. different types of influenza or different strands of the same microbes). We assume that two genomes G₁ and G₂ almost coincide and differ only in m randomly located nucleotides. This situation simulates the existence of point mutations or single nucleotide polymorphisms (SNPs). Let L be the size (number of probes) of the microarray and p—the frequency of presence of n-mers in a genome with a TSL value M. The value of L, necessary to distinguish the fingerprints of these two genomes with the error probability ε, can be estimated by the formula (see Appendix B):

$$L = \frac{log ɛ}{p log ( 1 - mn / N )} \leq \frac{M \vert log ɛ \vert}{pmn} . \qquad 5.5 )$$

Such an approach can provide the level of accuracy necessary for the individual human fingerprints. Let us assume that the differences between individual human beings appear only because of SNPs, which have equal probability and are randomly located in the genome. According to literature estimates [23], the total number of SNPs in the human genome is expected to be around 3,000,000. Then, calculating the necessary size for the random microarray (m/M˜0.1%, ε=10°, n=17, p=0.284), we have L˜4769. This rough estimation is promising and indicates that this possibility deserves a proper experimental study. We would like to recall, that our theoretical estimations have been made for randomly picked sets of n-mers. The further possibility exists to start with a larger than necessary random set of n-mers (say, L=10000) and then to decrease the microarray size experimenting with the desirable set of genomes (using, for instance, a simple optimization approach).

### Random Sets and Composite Probes

Research conducted at the University of Houston has focused on understanding the presence/absence of short subsequences in various genomes. The study of these short subsequences, also referred to as n-mers where n corresponds to the length of the subsequence, has revealed some interesting results. Using existing algorithms, analysis has been performed for more than 250 microbial, viral and multicellular organisms. A remarkable similarity of the shape of presence/absence distributions for different n-mers in all genomes was found. The same analysis applied analytically and numerically to random sequences also shows a similar shape of the distribution (the “random boundary”), with differences that correlate with biology. This led to the hypothesis that the presence/absence distribution of n-mers in all genomes considered (provided that the condition M<4ⁿ holds, where M is the total genome sequence length) can be treated as nearly random. A correlation analysis of distributions of the presence/absence of short subsequences of different length (n-mers, n=5-20) in 250 published microbial and virus genomes was also performed. The results show that for organisms, which are not close relatives of each other, the presence/absence of different 7-17-mers in their genomes is practically uncorrelated.

The low correlation among the n-mers present in different genomes suggests that it is possible to use random sets of n-mers (with appropriately chosen n) to discriminate between different microbial and viral genomes. Using a single appropriately chosen set of n-mers for each organism category (e.g. bacteria, viruses or humans), one would experimentally determine the presence or absence of every n-mer in the organism of interest. The important advantage of this approach is that it can be used without any a priori knowledge of the genomic sequence of the target organism.

This idea to use random sets of n-mers cannot be applied, as is, to samples of mixed organism categories, i.e. organisms with dramatically different genome lengths. A mixed sample of microbial, virus, and human, all belonging to different categories, would require conflicting values of n. For instance, an n-mer of length 16 or 17 can distinguish the human genome; however, in smaller genomes such as a microbial genome, there are very few 16- or 17-mers present. An n-mer of size 12 can distinguish a microbial but when applied to the human genome, nearly all 12-mers are present. In the context of probe design for a microarray assay, if 12-mers were used as probes, in the presence of a mixed sample such as a microbial and human mix, nearly all probes would bind to targets. The image produced by the microarray would appear very dark offering little information about the contents of the sample. On the other hand, if 16- or 17-mers were used, those probes specific to the larger human genome would bind; however, because very few probes would bind to the smaller microbial genome, the microarray image would appear very sparse if not completely blank. The case is even more desolate for the detection of a virus having an even smaller genome.

To discover the presence of a smaller genome(s) in a mixed sample, we must first determine what n-mers are present in the smaller genome(s) that are not present in the larger. When designing probes, we want to only include those n-mers not found in the larger genome. There can, however, be many n-mers meeting this criterion. Thus a problem arises: we need to guarantee that the microarray is dense enough to include many n-mers not found in the larger genome. A microarray is normally spotted with one particular probe at each position on the slide. Following this traditional approach, a majority of the n-mers unique to the smaller genome(s) which we are trying to detect must be included in the probe set. Assuring an appropriate density for the microarray causes a large scale and, more than likely, expensive experiment.

A simple yet unique solution to this problem requires a new method in probe design. Instead of affixing one particular probe to each “spot” on a microarray chip, multiple probes, or as we call them, composite probes can be used. By placing 10 to 100 different probes, the probability of locating a small genome in a mixed sample increases 10- to 100-fold without enlarging the chip size. Selecting the group of probes to be “spotted” together will be practically random; selection will necessitate some analysis of the group to verify that the probes will be able to bind to the target, i.e. they will not bind to each other. Moreover, it is not of concern if more than one target binds to a probe site. The properties of composite probes will be the same as those of a single probe for a microarray experiment.

Microarray experiments with composite probes are ideal for diagnosis. Quasi-random probes can be selected, grouped, and spotted to identify the presence of smaller genomes in mixed and unknown samples. In order to successfully use composite probes, the following two assumptions must preferably hold true:
1. the genomes which we will be detecting should adhere to the properties of independence which we have found to be true in past research, and
2. enough probes are used on the microarray chip to make visible results likely; we in turn can control the percentage of visibility by the number of probes “spotted” together.
   Given that these conditions are met, the use of composite probe arrays can be an invaluable diagnostic tool.

In many cases, an efficient way to make composite probes is to incorporate positions at which mixed bases are incorporated during synthesis. A single position made to be 90% A and 10% G, for example, doubles the number of probes in a spot or on a particle. A single sequence far from the closes background sequence can be expanded into a large number (thousands) of probes by mixed synthesis at more than one position, taking into account the possibility of mixing at different locations. For example, there are 20 locations at which to introduce mixed synthesis into a 20-mer, but a very large number of possible arrangements of three modification positions in a 20-mer.

Other application of host- or background-blind probes and primers include the creation of labels known to have reduced probability of occurring in the background, detection of food (e.g. meat) adulteration, detection of human genetic rearrangements and mutations, monitoring of xenotransplant organs and patients, and enhancement of identification methods based on rRNA and intergenic regions.

### Correlations

N-mers specific for identifying specific organisms are on the accompanying CDs.

Table of Contents
CD1 contains two folders - “SEQ ID NO 1: microbial” and SEQ ID NO 2:
“Human_mismatches”.
SEQ ID NO 1Folder Microbial - It contains 164 Microbial blind files, containing
sequences which are present in Microbial genome but are not present in Human genome
even with two mismatches. The list below gives us the file name and the corresponding
Microbial name. [Individual files can be referred to by the SEQ ID followed by the file
number, as in “SEQ ID-1” which refers to the file immediately below.]

| | File Name | Microbial Name |
|---|---|---|
| 1) | 18mers_human_m_2MM_1_p.txt | Aeropyrum pernix K1 complete genome. |
| 2) | 18mers_human_m_2MM_2_p.txt | Agrobacterium tumefaciens str. C58 linear |
| | | chromosome |
| 3) | 18mers_human_m_2MM_3_p.txt | Agrobacterium tumefaciens str. C58 AT |
| | | plasmid complete sequence. |
| 4) | 18mers_human_m_2MM_4_p.txt | Agrobacterium tumefaciens str. C58 Ti |
| | | plasmid |
| 5) | 18mers_human_m_2MM_5_p.txt | Agrobacterium tumefaciens strain C58 |
| | | circular chromosome |
| 6) | 18mers_human_m_2MM_6_p.txt | Agrobacterium tumefaciens strain C58 |
| | | circular chromosome |
| 7) | 18mers_human_m_2MM_7_p.txt | Agrobacterium tumefaciens strain C58 |
| | | linear chromosome |
| 8) | 18mers_human_m_2MM_8_p.txt | Agrobacterium tumefaciens strain C58 |
| | | plasmid AT |
| 9) | 18mers_human_m_2MM_9_p.txt | Agrobacterium tumefaciens strain C58 |
| | | plasmid Ti |
| 10) | 18mers_human_m_2MM_10_p.txt | Aquifex aeolicus complete genome. |
| 11) | 18mers_human_m_2MM_11_p.txt | Aquifex aeolicus plasmid ece1 |
| 12) | 18mers_human_m_2MM_12_p.txt | Archaeoglobus fulgidus complete genome. |
| 13) | 18mers_human_m_2MM_13_p.txt | Bacillus halodurans C-125 |
| 14) | 18mers_human_m_2MM_14_p.txt | Bacillus subtilis complete genome. |
| 15) | 18mers_human_m_2MM_15_p.txt | Bifidobacterium longum NCC2705 |
| | | complete genome. |
| 16) | 18mers_human_m_2MM_16_p.txt | Borrelia burgdorferi. |
| 17) | 18mers_human_m_2MM_21_p.txt | Borrelia burgdorferi plasmid cp32-4 |
| 18) | 18mers_human_m_2MM_22_p.txt | Borrelia burgdorferi plasmid cp32-6 |
| 19) | 18mers_human_m_2MM_26_p.txt | Borrelia burgdorferi plasmid lp5 |
| 20) | 18mers_human_m_2MM_31_p.txt | Borrelia burgdorferi plasmid lp28-2 |
| 21) | 18mers_human_m_2MM_32_p.txt | Borrelia burgdorferi plasmid lp28-3 |
| 22) | 18mers_human_m_2MM_33_p.txt | Borrelia burgdorferi plasmid lp28-4 |
| 23) | 18mers_human_m_2MM_34_p.txt | Borrelia burgdorferi plasmid lp36 |
| 24) | 18mers_human_m_2MM_36_p.txt | Borrelia burgdorferi plasmid lp54 |
| 25) | 18mers_human_m_2MM_38_p.txt | Bradyrhizobium japonicum strain |
| | | USDA110 |
| 26) | 18mers_human_m_2MM_39_p.txt | Brucella melitensis strain 16M chromosome I |
| 27) | 18mers_human_m_2MM_40_p.txt | Brucella suis 1330 chromosome I complete |
| | | genome. |
| 28) | 18mers_human_m_2MM_41_p.txt | Buchnera aphidicola str. Sg (Schizaphis |
| | | graminum) complete genome. |
| 29) | 18mers_human_m_2MM_42_p.txt | Buchnera sp. APS complete genome. |
| 30) | 18mers_human_m_2MM_44_p.txt | Buchnera sp. APS plasmid pTrp DNA |
| 31) | 18mers_human_m_2MM_45_p.txt | Campylobacter jejuni complete genome. |
| 32) | 18mers_human_m_2MM_46_p.txt | Caulobacter crescentus complete genome. |
| 33) | 18mers_human_m_2MM_47_p.txt | Chlamydia muridarum |
| 34) | 18mers_human_m_2MM_48_p.txt | Chlamydia muridarum plasmid pMoPn |
| 35) | 18mers_human_m_2MM_49_p.txt | Chlamydia trachomatis complete genome. |
| 36) | 18mers_human_m_2MM_50_p.txt | Chlamydophila pneumoniae AR39 |
| 37) | 18mers_human_m_2MM_51_p.txt | Chlamydophila pneumoniae J138 |
| 38) | 18mers_human_m_2MM_52_p.txt | Chlorobium tepidum TLS complete |
| | | genome. |
| 39) | 18mers_human_m_2MM_53_p.txt | Clostridium acetobutylicum ATCC824 |
| | | complete genome. |
| 40) | 18mers_human_m_2MM_54_p.txt | Clostridium acetobutylicum megaplasmid |
| | | pSOL1 |
| 41) | 18mers_human_m_2MM_55_p.txt | Clostridium perfringens 13 DNA |
| 42) | 18mers_human_m_2MM_57_p.txt | Corynebacterium efficiens YS-314 |
| 43) | 18mers_human_m_2MM_58_p.txt | Corynebacterium glutamicum ATCC 13032 |
| 44) | 18mers_human_m_2MM_59_p.txt | Deinococcus radiodurans R1 complete |
| | | chromosome 1. |
| 45) | 18mers_human_m_2MM_60_p.txt | Deinococcus radiodurans R1 megaplasmid |
| | | MP1 |
| 46) | 18mers_human_m_2MM_61_p.txt | Deinococcus radiodurans R1 plasmid CP1 |
| 47) | 18mers_human_m_2MM_62_p.txt | Escherichia coli CFT073 complete genome. |
| 48) | 18mers_human_m_2MM_63_p.txt | Escherichia coli K-12 MG1655 complete |
| | | genome. |
| 49) | 18mers_human_m_2MM_64_p.txt | Escherichia coli O157:H7 |
| 50) | 18mers_human_m_2MM_65_p.txt | Escherichia coli O157:H7 |
| 51) | 18mers_human_m_2MM_66_p.txt | Fusobacterium nucleatum subsp. nucleatum |
| | | ATCC 25586 |
| 52) | 18mers_human_m_2MM_67_p.txt | Haemophilus influenzae Rd complete |
| | | genome. |
| 53) | 18mers_human_m_2MM_68_p.txt | Halobacterium sp. NRC-1 complete |
| | | genome. |
| 54) | 18mers_human_m_2MM_69_p.txt | Halobacterium sp. NRC-1 plasmid |
| | | pNRC100 |
| 55) | 18mers_human_m_2MM_70_p.txt | Halobacterium sp. NRC-1 plasmid |
| | | pNRC200 complete genome. |
| 56) | 18mers_human_m_2MM_71_p.txt | Helicobacter pylori |
| 57) | 18mers_human_m_2MM_72_p.txt | Helicobacter pylori 26695 complete |
| | | genome. |
| 58) | 18mers_human_m_2MM_73_p.txt | Lactococcus lactis subsp. lactis IL1403 |
| | | complete genome. |
| 59) | 18mers_human_m_2MM_74_p.txt | Leptospira interrogans serovar lai str. 56601 |
| | | chromosome I |
| 60) | 18mers_human_m_2MM_75_p.txt | Listeria innocua Clip11262 |
| 61) | 18mers_human_m_2MM_76_p.txt | Listeria innocua plasmid pL1100 |
| 62) | 18mers_human_m_2MM_77_p.txt | Listeria monocytogenes strain EGD |
| 63) | 18mers_human_m_2MM_78_p.txt | Mesorhizobium loti complete genome |
| 64) | 18mers_human_m_2MM_79_p.txt | Mesorhizobium loti plasmid pMLa DNA |
| 65) | 18mers_human_m_2MM_80_p.txt | Mesorhizobium loti plasmid pMLb DNA |
| 66) | 18mers_human_m_2MM_81_p.txt | Methanobacterium thermoautotrophicum |
| | | delta H complete genome. |
| 67) | 18mers_human_m_2MM_82_p.txt | Methanococcus jannaschii complete |
| | | genome. |
| 68) | 18mers_human_m_2MM_83_p.txt | Methanococcus jannaschii large extra- |
| | | chromosomal element |
| 69) | 18mers_human_m_2MM_85_p.txt | Methanopyrus kandleri AV19 |
| 70) | 18mers_human_m_2MM_86_p.txt | Methanosarcina acetivorans str. C2A |
| 71) | 18mers_human_m_2MM_87_p.txt | Methanosarcina mazei strain Goe1 |
| 72) | 18mers_human_m_2MM_88_p.txt | Mycobacterium leprae strain TN complete |
| | | genome. |
| 73) | 18mers_human_m_2MM_89_p.txt | Mycobacterium tuberculosis CDC1551 |
| 74) | 18mers_human_m_2MM_90_p.txt | Mycobacterium tuberculosis complete |
| | | genome. |
| 75) | 18mers_human_m_2MM_91_p.txt | Mycoplasma genitalium G37 complete |
| | | genome. |
| 76) | 18mers_human_m_2MM_92_p.txt | Mycoplasma penetrans |
| 77) | 18mers_human_m_2MM_93_p.txt | Mycoplasma pneumoniae M129 |
| 78) | 18mers_human_m_2MM_94_p.txt | Mycoplasma pulmonis (strain UAB CTIP) |
| | | complete genome. |
| 79) | 18mers_human_m_2MM_95_p.txt | Neisseria meningitidis serogroup A strain |
| | | Z2491 complete genome. |
| 80) | 18mers_human_m_2MM_96_p.txt | Neisseria meningitidis serogroup B strain |
| | | MC58 complete genome. |
| 81) | 18mers_human_m_2MM_97_p.txt | Nostoc sp. PCC 7120 complete genome. |
| 82) | 18mers_human_m_2MM_98_p.txt | Nostoc sp. PCC 7120 plasmid |
| | | pCC7120alpha DNA |
| 83) | 18mers_human_m_2MM_99_p.txt | Nostoc sp. PCC 7120 plasmid |
| | | pCC7120beta DNA |
| 84) | 18mers_human_m_2MM_100_p.txt | Nostoc sp. PCC 7120 plasmid |
| | | pCC7120delta DNA |
| 85) | 18mers_human_m_2MM_101_p.txt | Nostoc sp. PCC 7120 plasmid |
| | | pCC7120epsilon DNA |
| 86) | 18mers_human_m_2MM_102_p.txt | Nostoc sp. PCC 7120 plasmid |
| | | pCC7120gamma DNA |
| 87) | 18mers_human_m_2MM_103_p.txt | Nostoc sp. PCC 7120 plasmid pCC7120zeta |
| | | DNA |
| 88) | 18mers_human_m_2MM_104_p.txt | Oceanobacillus iheyensis |
| 89) | 18mers_human_m_2MM_105_p.txt | Pasteurella multocida PM70 complete |
| | | genome. |
| 90) | 18mers_human_m_2MM_106_p.txt | Pseudomonas aeruginosa PA01 |
| 91) | 18mers_human_m_2MM_107_p.txt | Pseudomonas putida KT2440 |
| 92) | 18mers_human_m_2MM_108_p.txt | Pyrobaculum aerophilum strain IM2 |
| | | complete genome. |
| 93) | 18mers_human_m_2MM_109_p.txt | Pyrococcus abyssi complete genome. |
| 94) | 18mers_human_m_2MM_110_p.txt | Pyrococcus furiosus DSM 3638 |
| 95) | 18mers_human_m_2MM_111_p.txt | Pyrococcus horikoshii OT3 complete |
| | | genome. |
| 96) | 18mers_human_m_2MM_112_p.txt | Ralstonia solanacearum GMI1000 |
| | | chromosome |
| 97) | 18mers_human_m_2MM_113_p.txt | Ralstonia solanacearum GMI1000 |
| | | megaplasmid |
| 98) | 18mers_human_m_2MM_114_p.txt | Rickettsia conorii Malish 7 |
| 99) | 18mers_human_m_2MM_115_p.txt | Rickettsia prowazekii strain Madrid E |
| 100) | 18mers_human_m_2MM_116_p.txt | Salmonella enterica serovar Typhi |
| | | (Salmonella typhi) strain CT18 |
| 101) | 18mers_human_m_2MM_117_p.txt | Salmonella enterica serovar Typhi |
| | | (Salmonella typhi) strain CT18 plasmid pHCM1 |
| 102) | 18mers_human_m_2MM_118_p.txt | Salmonella enterica serovar Typhi |
| | | (Salmonella typhi) strain CT18 plasmid pHCM2 |
| 103) | 18mers_human_m_2MM_119_p.txt | Salmonella typhimurium LT2 |
| 104) | 18mers_human_m_2MM_120_p.txt | Salmonella typhimurium LT2 strain |
| | | SGSC1412 plasmid pSLT complete |
| 105) | 18mers_human_m_2MM_121_p.txt | Shewanella oneidensis MR-1 complete |
| | | genome. |
| 106) | 18mers_human_m_2MM_122_p.txt | Shewanella oneidensis MR-1 megaplasmid |
| | | complete sequence. |
| 107) | 18mers_human_m_2MM_123_p.txt | Shigella flexneri 2a str. 301 complete |
| | | genome. |
| 108) | 18mers_human_m_2MM_124_p.txt | Sinorhizobium meliloti 1021 complete |
| | | genomes. |
| 109) | 18mers_human_m_2MM_125_p.txt | Sinorhizobium meliloti plasmid pSymA |
| | | complete plasmid sequence. |
| 110) | 18mers_human_m_2MM_126_p.txt | Sinorhizobium meliloti plasmid pSymB |
| 111) | 18mers_human_m_2MM_127_p.txt | Staphylococcus aureus MW2 |
| 112) | 18mers_human_m_2MM_128_p.txt | Staphylococcus aureus strain Mu50 |
| 113) | 18mers_human_m_2MM_129_p.txt | Staphylococcus aureus plasmid VRSAp |
| 114) | 18mers_human_m_2MM_130_p.txt | Staphylococcus aureus strain N315 |
| 115) | 18mers_human_m_2MM_131_p.txt | Staphylococcus aureus subsp. aureus N315 |
| | | plasmid pN315B DNA |
| 116) | 18mers_human_m_2MM_132_p.txt | Streptococcus agalactiae |
| 117) | 18mers_human_m_2MM_133_p.txt | Streptococcus agalactiae NEM316 |
| 118) | 18mers_human_m_2MM_134_p.txt | Streptococcus mutans UA159 complete |
| | | genome. |
| 119) | 18mers_human_m_2MM_135_p.txt | Streptococcus pneumoniae complete |
| | | genome. |
| 120) | 18mers_human_m_2MM_136_p.txt | Streptococcus pneumoniae R6 complete |
| | | genome. |
| 121) | 18mers_human_m_2MM_137_p.txt | Streptococcus pyogenes MGAS315 |
| 122) | 18mers_human_m_2MM_138_p.txt | Streptococcus pyogenes strain MGAS8232 |
| 123) | 18mers_human_m_2MM_139_p.txt | Streptococcus pyogenes strain SF370 |
| | | serotype M1 |
| 124) | 18mers_human_m_2MM_140_p.txt | Streptomyces coelicolor A3(2) |
| 125) | 18mers_human_m_2MM_141_p.txt | Streptomyces coelicolor plasmid SCP1. |
| 126) | 18mers_human_m_2MM_142_p.txt | Streptomyces coelicolor plasmid SCP2. |
| 127) | 18mers_human_m_2MM_143_p.txt | Sulfolobus solfataricus complete genome. |
| 128) | 18mers_human_m_2MM_144_p.txt | Sulfolobus tokodaii complete genome. |
| 129) | 18mers_human_m_2MM_145_p.txt | Synechocystis PCC6803 complete genome. |
| 130) | 18mers_human_m_2MM_146_p.txt | Thermoanaerobacter tengcongensis strain |
| | | MB4T |
| 131) | 18mers_human_m_2MM_147_p.txt | Thermoplasma acidophilum |
| 132) | 18mers_human_m_2MM_148_p.txt | Thermoplasma volcanium |
| 133) | 18mers_human_m_2MM_149_p.txt | Thermosynechococcus elongatus BP-1 |
| 134) | 18mers_human_m_2MM_150_p.txt | Thermotoga maritima complete genome. |
| 135) | 18mers_human_m_2MM_151_p.txt | Treponema pallidum complete genome. |
| 136) | 18mers_human_m_2MM_152_p.txt | Ureaplasma urealyticum complete genome. |
| 137) | 18mers_human_m_2MM_153_p.txt | Vibrio cholerae chromosome I |
| 138) | 18mers_human_m_2MM_154_p.txt | Vibrio vulnificus CMCP6 chromosome I |
| | | complete sequence. |
| 139) | 18mers_human_m_2MM_155_p.txt | Vibrio parahaemolyticus RIMD 2210633, |
| | | chromosome 1, complete sequence |
| 140) | 18mers_human_m_2MM_156_p.txt | Wigglesworthia brevipalpis |
| 141) | 18mers_human_m_2MM_157_p.txt | Xanthomonas axonopodis pv. citri str. 306 |
| 142) | 18mers_human_m_2MM_158_p.txt | Xylella fastidiosa Temeculal |
| 143) | 18mers_human_m_2MM_159_p.txt | Xanthomonas campestris pv. campestris str. |
| | | ATCC 33913 |
| 144) | 18mers_human_m_2MM_160_p.txt | Xylella fastidiosa |
| 145) | 18mers_human_m_2MM_161_p.txt | Xylella fastidiosa plasmid pXF1.3 |
| 146) | 18mers_human_m_2MM_162_p.txt | Xylella fastidiosa plasmid pXF51 |
| 147) | 18mers_human_m_2MM_163_p.txt | Xylella fastidiosa plasmid pXF868 |
| 148) | 18mers_human_m_2MM_164_p.txt | Yersinia pestis KIM complete genome. |
| 149) | 18mers_human_m_2MM_165_p.txt | Yersinia pestis strain CO92 |
| 150) | 18mers_human_m_2MM_166_p.txt | Yersinia pestis plasmid pCD1. |
| 151) | 18mers_human_m_2MM_167_p.txt | Yersinia pestis plasmid pPCP1. |
| 152) | 18mers_human_m_2MM_168_p.txt | Yersinia pestis plasmid pPMT1. |
| 153) | 18mers_human_m_2MM_169_p.txt | Chlamydophila pneumoniae CWL029 |
| | | complete genome |
| 154) | 18mers_human_m_2MM_170_p.txt | Nitrosomonas europaea |
| 155) | 18mers_human_m_2MM_171_p.txt | Prochlorococcus marinus MED4 |
| 156) | 18mers_human_m_2MM_172_p.txt | Prochlorococcus marinus MIT9313 |
| 157) | 18mers_human_m_2MM_173_p.txt | Rhodopseudomonas palustris |
| 158) | 18mers_human_m_2MM_174_p.txt | Staphylococcus epidermidis ATCC 12228, |
| | | complete genome |
| 159) | 18mers_human_m_2MM_175_p.txt | Synechococcus |
| 160) | 18mers_human_m_2MM_176_p.txt | Clostridium tetani E88, complete genome. |
| 161) | 18mers_human_m_2MM_177_p.txt | Lactobacillus plantarum WCFS1, complete |
| | | genome. |
| 162) | 18mers_human_m_2MM_178_p.txt | Pseudomonas syringae pv. tomato str. |
| | | DC3000 |
| 163) | 18mers_human_m_2MM_179_p.txt | Streptococcus pyogenes phage 315.3 |
| | | provirus |
| 164) | 18mers_human_m_2MM_180_p.txt | Tropheryma whipplei str. Twist |

SEQ ID NO 2: Folder human_mismatches - This folder contains 4 files that contain
sequences which are not present in Human genome.

| | |
|---|---|
| 1) | “All human blind 11-mers” - this file contains 94 sequences of length eleven, |
| | which are not present in Human genome. |
| 2) | “All human blind 12-mers” - this file contains 44266 sequences of length twelve, |
| | which are not present in Human genome. |
| 3) | “All human blind 13-mers_1.txt” - this file contains first 170000 sequences of |
| | length 13 which are not present in human genome. |
| 4) | “All human blind 13-mers_2.txt” - this file contains next 682362 sequences of |
| | length 13 which are not present in human genome. |

CD2 has a single folder SEQ ID NO 3: “human_mismatches”.
SEQ ID NO 3: Folder human_mismatches - This folder has 25 files which contain
sequences of length 14, which are not present in Human genome. There are in total
41294373 sequences which are not present in Human genome. These sequences are in 25
files, the first 24 contain 1700000 sequences each and the last file contains 494373
sequences. The list of file is as follows -

| | |
|---|---|
| 1) | “All human blind 14-mers_1.txt” |
| 2) | “All human blind 14-mers_2.txt” |
| 3) | “All human blind 14-mers_3.txt” |
| 4) | “All human blind 14-mers_4.txt” |
| 5) | “All human blind 14-mers_5.txt” |
| 6) | “All human blind 14-mers_6.txt” |
| 7) | “All human blind 14-mers_7.txt” |
| 8) | “All human blind 14-mers_8.txt” |
| 9) | “All human blind 14-mers_9.txt” |
| 10) | “All human blind 14-mers_10.txt” |
| 11) | “All human blind 14-mers_11.txt” |
| 12) | “All human blind 14-mers_12.txt” |
| 13) | “All human blind 14-mers_13.txt” |
| 14) | “All human blind 14-mers_14.txt” |
| 15) | “All human blind 14-mers_15.txt” |
| 16) | “All human blind 14-mers_16.txt” |
| 17) | “All human blind 14-mers_17.txt” |
| 18) | “All human blind 14-mers_18.txt” |
| 19) | “All human blind 14-mers_19.txt” |
| 20) | “All human blind 14-mers_20.txt” |
| 21) | “All human blind 14-mers_21.txt” |
| 22) | “All human blind 14-mers_22.txt” |
| 23) | “All human blind 14-mers_23.txt” |
| 24) | “All human blind 14-mers_24.txt” |
| 25) | “All human blind 14-mers_25.txt” |

CD3 - contains six folders - SEQ ID NO 4: “Virus 18 mers 2MM”, SEQ ID NO 5:
“Virus 18 mers 1MM”, SEQ ID NO 6: “Virus 17 mers 2MM”, SEQ ID NO 7: “Virus
17 mers 1MM”, SEQ ID NO 8: ““Selected Microbe 17 mers” and SEQ ID NO
9: “Selected Microbe 18 mers”. CD3 contains six folders—SEQ ID NO 4:”
1) SEQ ID NO 4: Virus 18 mers 2MM - It contains 997 files, containing sequences
of length 18, which are present in the viral genome, but are not present in the
human genome. All sequences produced from these sequences with two
mismatches are also not present in the human genome. The list below contains
the file name and the corresponding virus name.

| | file name | Species Name |
|---|---|---|
| 1) | 18mers_human_m_virus_2MM_14_p.txt | Peanut chlorotic streak virus, complete genome. |
| 2) | 18mers_human_m_virus_2MM_19_p.txt | Sugarcane bacilliform virus, complete genome. |
| 3) | 18mers_human_m_virus_2MM_21_p.txt | Tobacco vein-clearing virus, complete genome. |
| 4) | 18mers_human_m_virus_2MM_24_p.txt | Ground squirrel hepatitis virus, complete genome. |
| 5) | 18mers_human_m_virus_2MM_29_p.txt | Woolly monkey hepatitis B Virus, complete |
| | | genome. |
| 6) | 18mers_human_m_virus_2MM_35_p.txt | Bovine foamy virus, complete genome. |
| 7) | 18mers_human_m_virus_2MM_38_p.txt | Caprine arthritis-encephalitis virus, complete |
| | | genome. |
| 8) | 18mers_human_m_virus_2MM_44_p.txt | Friend murine leukemia virus, complete genome. |
| 9) | 18mers_human_m_virus_2MM_61_p.txt | Ovine lentivirus, complete genome. |
| 10) | 18mers_human_m_virus_2MM_64_p.txt | Rauscher murine leukemia virus, complete genome. |
| 11) | 18mers_human_m_virus_2MM_72_p.txt | Snakehead retrovirus, complete genome. |
| 12) | 18mers_human_m_virus_2MM_84_p.txt | Chicory yellow mottle virus large satellite RNA, |
| | | complete genome. |
| 13) | 18mers_human_m_virus_2MM_89_p.txt | Eupatorium yellow vein virus-associated DNA beta, |
| | | complete genome. |
| 14) | 18mers_human_m_virus_2MM_93_p.txt | Malvastrum yellow vein virus-associated DNA beta, |
| | | complete genome. |
| 15) | 18mers_human_m_virus_2MM_102_p.txt | Satellite virus of maize white line mosaic virus, |
| | | complete genome. |
| 16) | 18mers_human_m_virus_2MM_103_p.txt | Satellite of Trichomonas vaginalis T1 virus, |
| | | complete genome. |
| 17) | 18mers_human_m_virus_2MM_110_p.txt | Tobacco curly shoot virus-associated DNA beta, |
| | | complete genome. |
| 18) | 18mers_human_m_virus_2MM_115_p.txt | Tomato bushy stunt virus satellite RNA B10, |
| | | complete genome. |
| 19) | 18mers_human_m_virus_2MM_116_p.txt | Tomato leaf curl virus satellite DNA, complete |
| | | genome. |
| 20) | 18mers_human_m_virus_2MM_285_p.txt | Canine oral papillomavirus, complete genome. |
| 21) | 18mers_human_m_virus_2MM_303_p.txt | Human papillomavirus type 16, complete genome. |
| 22) | 18mers_human_m_virus_2MM_320_p.txt | Human papillomavirus type 30, complete genome. |
| 23) | 18mers_human_m_virus_2MM_330_p.txt | Human papillomavirus type 4, complete genome. |
| 24) | 18mers_human_m_virus_2MM_334_p.txt | Human papillomavirus type 44, complete genome. |
| 25) | 18mers_human_m_virus_2MM_336_p.txt | Human papillomavirus type 47, complete genome. |
| 26) | 18mers_human_m_virus_2MM_337_p.txt | Human papillomavirus type 48, complete genome. |
| 27) | 18mers_human_m_virus_2MM_345_p.txt | Human papillomavirus type 55, complete genome. |
| 28) | 18mers_human_m_virus_2MM_357_p.txt | Human papillomavirus type 67, complete genome. |
| 29) | 18mers_human_m_virus_2MM_368_p.txt | Human papillomavirus type 84, complete genome. |
| 30) | 18mers_human_m_virus_2MM_372_p.txt | Human papillomavirus type 9, complete genome. |
| 31) | 18mers_human_m_virus_2MM_394_p.txt | Murine polyomavirus strain A2, complete genome. |
| 32) | 18mers_human_m_virus_2MM_413_p.txt | Sulfolobus virus SIRV-2, complete genome. |
| 33) | 18mers_human_m_virus_2MM_643_p.txt | TTV-like mini virus, complete genome. |
| 34) | 18mers_human_m_virus_2MM_661_p.txt | Bean yellow dwarf virus, complete genome. |
| 35) | 18mers_human_m_virus_2MM_663_p.txt | Beet mild curly top virus, complete genome. |
| 36) | 18mers_human_m_virus_2MM_683_p.txt | Dicliptera yellow mottle virus DNA A, complete |
| | | sequence. |
| 37) | 18mers_human_m_virus_2MM_731_p.txt | Sida golden mosaic Florida virus, complete genome. |
| 38) | 18mers_human_m_virus_2MM_763_p.txt | Tobacco leaf curl Yunnan virus, complete genome. |
| 39) | 18mers_human_m_virus_2MM_765_p.txt | Tobacco yellow dwarf virus, complete genome. |
| 40) | 18mers_human_m_virus_2MM_766_p.txt | Tomato chlorotic mottle virus DNA A, complete |
| | | sequence. |
| 41) | 18mers_human_m_virus_2MM_778_p.txt | Tomato leaf curl Malaysia virus, complete genome. |
| 42) | 18mers_human_m_virus_2MM_796_p.txt | Tomato yellow leaf curl Gezira virus, complete |
| | | genome. |
| 43) | 18mers_human_m_virus_2MM_801_p.txt | Tomato yellow leaf curl virus, complete genome. |
| 44) | 18mers_human_m_virus_2MM_815_p.txt | Spiroplasma phage 1-C74, complete genome. |
| 45) | 18mers_human_m_virus_2MM_817_p.txt | SVTS2 plectrovirus, complete genome. |
| 46) | 18mers_human_m_virus_2MM_832_p.txt | Spiroplasma phage 4, complete genome. |
| 47) | 18mers_human_m_virus_2MM_833_p.txt | Ageratum yellow vein virus-associated nanovirus |
| | | DNA, complete genome. |
| 48) | 18mers_human_m_virus_2MM_883_p.txt | Canine parvovirus, complete genome. |
| 49) | 18mers_human_m_virus_2MM_888_p.txt | Human erythrovirus V9, complete genome. |
| 50) | 18mers_human_m_virus_2MM_892_p.txt | Mice minute virus, complete genome. |
| 51) | 18mers_human_m_virus_2MM_894_p.txt | Mouse parvovirus 1, complete genome. |
| 52) | 18mers_human_m_virus_2MM_941_p.txt | Human parainfluenza virus 2, complete genome. |
| 53) | 18mers_human_m_virus_2MM_1008_p.txt | Rice grassy stunt virus RNA 1, complete sequence. |
| 54) | 18mers_human_m_virus_2MM_1014_p.txt | Rice stripe virus RNA 1, complete sequence. |
| 55) | 18mers_human_m_virus_2MM_1152_p.txt | Plautia stali intestine virus, complete genome. |
| 56) | 18mers_human_m_virus_2MM_1155_p.txt | Triatoma virus, complete genome. |
| 57) | 18mers_human_m_virus_2MM_1170_p.txt | Modoc virus, genome. |
| 58) | 18mers_human_m_virus_2MM_1178_p.txt | Rio Bravo virus, genome. |
| 59) | 18mers_human_m_virus_2MM_1183_p.txt | Apple stem pitting virus, complete genome. |
| 60) | 18mers_human_m_virus_2MM_1236_p.txt | Cryphonectria parasitica mitovirus 1-N B631, |
| | | complete genome. |
| 61) | 18mers_human_m_virus_2MM_1291_p.txt | Human parechovirus 2, complete genome. |
| 62) | 18mers_human_m_virus_2MM_1292_p.txt | Human rhinovirus 89, complete genome. |
| 63) | 18mers_human_m_virus_2MM_1324_p.txt | Clover yellow vein virus, complete genome. |
| 64) | 18mers_human_m_virus_2MM_1358_p.txt | Apple latent spherical virus segment 1, complete |
| | | sequence. |
| 65) | 18mers_human_m_virus_2MM_1448_p.txt | Eggplant mosaic virus, complete genome. |
| 66) | 18mers_human_m_virus_2MM_1463_p.txt | Citrus leaf blotch virus, complete genome. |
| 67) | 18mers_human_m_virus_2MM_4_p.txt | Cacao swollen shoot virus, complete genome. |
| 68) | 18mers_human_m_virus_2MM_5_p.txt | Carnation etched ring virus, complete genome. |
| 69) | 18mers_human_m_virus_2MM_16_p.txt | Rice tungro bacilliform virus, complete genome. |
| 70) | 18mers_human_m_virus_2MM_20_p.txt | Taro bacilliform virus, complete genome. |
| 71) | 18mers_human_m_virus_2MM_23_p.txt | Duck hepatitis B virus, complete genome. |
| 72) | 18mers_human_m_virus_2MM_26_p.txt | Heron hepatitis B virus, complete genome. |
| 73) | 18mers_human_m_virus_2MM_36_p.txt | Bovine immunodeficiency virus, complete genome. |
| 74) | 18mers_human_m_virus_2MM_39_p.txt | Equine foamy virus, complete genome. |
| 75) | 18mers_human_m_virus_2MM_50_p.txt | Human spumaretrovirus, complete genome. |
| 76) | 18mers_human_m_virus_2MM_54_p.txt | Mason-Pfizer monkey virus, complete genome. |
| 77) | 18mers_human_m_virus_2MM_63_p.txt | Porcine endogenous retrovirus, complete genome. |
| 78) | 18mers_human_m_virus_2MM_79_p.txt | Arabis mosaic virus satellite RNA, complete |
| | | genome. |
| 79) | 18mers_human_m_virus_2MM_91_p.txt | Hollyhock leaf crumple virus satellite DNA, |
| | | complete genome. |
| 80) | 18mers_human_m_virus_2MM_94_p.txt | OkLCV satDNA 10, complete genome. |
| 81) | 18mers_human_m_virus_2MM_95_p.txt | OkLCV satDNA 27, complete genome. |
| 82) | 18mers_human_m_virus_2MM_98_p.txt | Papaya leaf curl virus-associated DNA beta, |
| | | complete genome. |
| 83) | 18mers_human_m_virus_2MM_108_p.txt | Strawberry latent ringspot virus satellite RNA, |
| | | complete genome. |
| 84) | 18mers_human_m_virus_2MM_111_p.txt | Tobacco necrosis satellite virus, complete genome. |
| 85) | 18mers_human_m_virus_2MM_112_p.txt | Tobacco ringspot virus satellite RNA, complete |
| | | genome. |
| 86) | 18mers_human_m_virus_2MM_117_p.txt | Tomato leaf curl virus-associated DNA beta, |
| | | complete genome. |
| 87) | 18mers_human_m_virus_2MM_135_p.txt | Ovine adenovirus 7, complete genome. |
| 88) | 18mers_human_m_virus_2MM_280_p.txt | Bovine papillomavirus, complete genome. |
| 89) | 18mers_human_m_virus_2MM_286_p.txt | Common chimpanzee papillomavirus 1, complete |
| | | genome. |
| 90) | 18mers_human_m_virus_2MM_292_p.txt | Feline papillomavirus, complete genome. |
| 91) | 18mers_human_m_virus_2MM_294_p.txt | Hamster papovavirus, complete genome. |
| 92) | 18mers_human_m_virus_2MM_300_p.txt | Human papillomavirus type 13, complete genome. |
| 93) | 18mers_human_m_virus_2MM_302_p.txt | Human papillomavirus type 15, complete genome. |
| 94) | 18mers_human_m_virus_2MM_309_p.txt | Human papillomavirus type 21, complete genome. |
| 95) | 18mers_human_m_virus_2MM_321_p.txt | Human papillomavirus type 31, complete genome. |
| 96) | 18mers_human_m_virus_2MM_328_p.txt | Human papillomavirus type 38, complete genome. |
| 97) | 18mers_human_m_virus_2MM_332_p.txt | Human papillomavirus type 41, complete genome. |
| 98) | 18mers_human_m_virus_2MM_350_p.txt | Human papillomavirus type 5b, complete genome. |
| 99) | 18mers_human_m_virus_2MM_362_p.txt | Human papillomavirus type 70, complete genome. |
| 100) | 18mers_human_m_virus_2MM_363_p.txt | Human papillomavirus type 71, complete |
| | | genome. |
| 101) | 18mers_human_m_virus_2MM_373_p.txt | Human papillomavirus type 90, complete |
| | | genome. |
| 102) | 18mers_human_m_virus_2MM_375_p.txt | Human papillomavirus type 92, complete |
| | | genome. |
| 103) | 18mers_human_m_virus_2MM_439_p.txt | Atkinsonella hypoxylon partitivirus RNA |
| | | 1, complete sequence. |
| 104) | 18mers_human_m_virus_2MM_634_p.txt | Diaporthe ambigua RNA virus 1, complete |
| | | genome. |
| 105) | 18mers_human_m_virus_2MM_642_p.txt | TT virus, complete genome. |
| 106) | 18mers_human_m_virus_2MM_646_p.txt | African cassava mosaic virus DNA A, |
| | | complete sequence. |
| 107) | 18mers_human_m_virus_2MM_648_p.txt | Ageratum enation virus, complete genome. |
| 108) | 18mers_human_m_virus_2MM_652_p.txt | Ageratum yellow vein virus, complete |
| | | genome. |
| 109) | 18mers_human_m_virus_2MM_708_p.txt | Miscanthus streak virus, complete genome. |
| 110) | 18mers_human_m_virus_2MM_721_p.txt | Pepper leaf curl virus DNA A, complete |
| | | sequence. |
| 111) | 18mers_human_m_virus_2MM_758_p.txt | Sweet potato leaf curl virus, complete |
| | | genome. |
| 112) | 18mers_human_m_virus_2MM_783_p.txt | Tomato leaf curl Vietnam virus, complete |
| | | genome. |
| 113) | 18mers_human_m_virus_2MM_826_p.txt | Chlamydia psittaci bacteriophage chp1, |
| | | complete genome. |
| 114) | 18mers_human_m_virus_2MM_889_p.txt | Infectious hypodermal and hematopoietic |
| | | necrosis virus, complete genome. |
| 115) | 18mers_human_m_virus_2MM_891_p.txt | LuIII virus, complete genome. |
| 116) | 18mers_human_m_virus_2MM_901_p.txt | Lymphocytic choriomeningitis virus |
| | | segment L, complete sequence. |
| 117) | 18mers_human_m_virus_2MM_911_p.txt | Dugbe virus segment L, complete |
| | | sequence. |
| 118) | 18mers_human_m_virus_2MM_926_p.txt | Tomato spotted wilt virus RNA L, |
| | | complete sequence. |
| 119) | 18mers_human_m_virus_2MM_940_p.txt | Human parainfluenza virus 1 strain |
| | | Washington/1964, complete genome. |
| 120) | 18mers_human_m_virus_2MM_944_p.txt | Infectious hematopoietic necrosis virus, |
| | | complete genome. |
| 121) | 18mers_human_m_virus_2MM_954_p.txt | Rice yellow stunt virus, complete genome. |
| 122) | 18mers_human_m_virus_2MM_1023_p.txt | Human astrovirus, complete genome. |
| 123) | 18mers_human_m_virus_2MM_1094_p.txt | Feline calicivirus, complete genome. |
| 124) | 18mers_human_m_virus_2MM_1100_p.txt | Apple stem grooving virus, complete |
| | | genome. |
| 125) | 18mers_human_m_virus_2MM_1132_p.txt | Grapevine chrome mosaic virus RNA 1, |
| | | complete sequence. |
| 126) | 18mers_human_m_virus_2MM_1136_p.txt | Patchouli mild mosaic virus RNA 1, |
| | | complete sequence. |
| 127) | 18mers_human_m_virus_2MM_1138_p.txt | Red clover mottle virus RNA 1, complete |
| | | sequence. |
| 128) | 18mers_human_m_virus_2MM_1157_p.txt | Apoi virus, genome. |
| 129) | 18mers_human_m_virus_2MM_1171_p.txt | Montana myotis leukoencephalitis virus, |
| | | complete genome. |
| 130) | 18mers_human_m_virus_2MM_1175_p.txt | Pestivirus type 2, complete genome. |
| 131) | 18mers_human_m_virus_2MM_1253_p.txt | Transmissible gastroenteritis virus, |
| | | complete genome. |
| 132) | 18mers_human_m_virus_2MM_1276_p.txt | Encephalomyocarditis virus, complete |
| | | genome. |
| 133) | 18mers_human_m_virus_2MM_1285_p.txt | Human echovirus 1, complete genome. |
| 134) | 18mers_human_m_virus_2MM_1286_p.txt | Human enterovirus A, complete genome. |
| 135) | 18mers_human_m_virus_2MM_1296_p.txt | Porcine enterovirus A, complete genome. |
| 136) | 18mers_human_m_virus_2MM_1298_p.txt | Porcine teschovirus 1, complete genome. |
| 137) | 18mers_human_m_virus_2MM_1299_p.txt | Simian picornavirus 1, complete genome. |
| 138) | 18mers_human_m_virus_2MM_1310_p.txt | Potato aucuba mosaic virus, complete |
| | | genome. |
| 139) | 18mers_human_m_virus_2MM_1322_p.txt | Bean yellow mosaic virus, complete |
| | | genome. |
| 140) | 18mers_human_m_virus_2MM_1336_p.txt | Pea seed-borne mosaic virus, complete |
| | | genome. |
| 141) | 18mers_human_m_virus_2MM_1350_p.txt | Tobacco vein mottling virus, complete |
| | | genome. |
| 142) | 18mers_human_m_virus_2MM_1433_p.txt | Panicum mosaic virus, complete genome. |
| 143) | 18mers_human_m_virus_2MM_1450_p.txt | Kennedya yellow mosaic virus, complete |
| | | genome. |
| 144) | 18mers_human_m_virus_2MM_1452_p.txt | Physalis mottle virus, complete genome. |
| 145) | 18mers_human_m_virus_2MM_1470_p.txt | Bacteriophage L5, complete genome. |
| 146) | 18mers_human_m_virus_2MM_1_p.txt | Hepatitis D virus, complete genome. |
| 147) | 18mers_human_m_virus_2MM_11_p.txt | Figwort mosaic virus, complete genome. |
| 148) | 18mers_human_m_virus_2MM_25_p.txt | Hepatitis B virus, complete genome. |
| 149) | 18mers_human_m_virus_2MM_41_p.txt | Feline foamy virus, complete genome. |
| 150) | 18mers_human_m_virus_2MM_58_p.txt | Murine osteosarcoma virus, complete |
| | | genome. |
| 151) | 18mers_human_m_virus_2MM_76_p.txt | Woolly monkey sarcoma virus, complete |
| | | genome. |
| 152) | 18mers_human_m_virus_2MM_83_p.txt | Cereal yellow dwarf virus-RPV satellite |
| | | RNA, complete genome. |
| 153) | 18mers_human_m_virus_2MM_96_p.txt | OkLCV satDNA 3, complete genome. |
| 154) | 18mers_human_m_virus_2MM_113_p.txt | Tomato black ring virus satellite RNA, |
| | | complete genome. |
| 155) | 18mers_human_m_virus_2MM_122_p.txt | Bovine adenovirus D, complete genome. |
| 156) | 18mers_human_m_virus_2MM_140_p.txt | Hemorrhagic enteritis virus, complete |
| | | genome. |
| 157) | 18mers_human_m_virus_2MM_250_p.txt | Bovine herpesvirus 4, complete genome. |
| 158) | 18mers_human_m_virus_2MM_284_p.txt | Bovine papillomavirus type 5, complete |
| | | genome. |
| 159) | 18mers_human_m_virus_2MM_287_p.txt | Cottontail rabbit papillomavirus, complete |
| | | genome. |
| 160) | 18mers_human_m_virus_2MM_314_p.txt | Human papillomavirus type 26, complete |
| | | genome. |
| 161) | 18mers_human_m_virus_2MM_335_p.txt | Human papillomavirus type 45, complete |
| | | genome. |
| 162) | 18mers_human_m_virus_2MM_340_p.txt | Human papillomavirus type 50, complete |
| | | genome. |
| 163) | 18mers_human_m_virus_2MM_342_p.txt | Human papillomavirus type 52, complete |
| | | genome. |
| 164) | 18mers_human_m_virus_2MM_356_p.txt | Human papillomavirus type 66, complete |
| | | genome. |
| 165) | 18mers_human_m_virus_2MM_366_p.txt | Human papillomavirus type 82, complete |
| | | genome. |
| 166) | 18mers_human_m_virus_2MM_370_p.txt | Human papillomavirus type 87, complete |
| | | genome. |
| 167) | 18mers_human_m_virus_2MM_384_p.txt | Rhesus monkey papillomavirus, complete |
| | | genome. |
| 168) | 18mers_human_m_virus_2MM_387_p.txt | Acholeplasma phage L2, complete |
| | | genome. |
| 169) | 18mers_human_m_virus_2MM_412_p.txt | Sulfolobus virus SIRV-1, complete |
| | | genome. |
| 170) | 18mers_human_m_virus_2MM_626_p.txt | Saccharomyces cerevisiae virus L-BC (La), |
| | | complete genome. |
| 171) | 18mers_human_m_virus_2MM_651_p.txt | Ageratum yellow vein Taiwan virus, |
| | | complete genome. |
| 172) | 18mers_human_m_virus_2MM_672_p.txt | Chloris striate mosaic virus, complete |
| | | genome. |
| 173) | 18mers_human_m_virus_2MM_696_p.txt | Horseradish curly top virus, complete |
| | | genome. |
| 174) | 18mers_human_m_virus_2MM_715_p.txt | Papaya leaf curl virus, complete genome. |
| 175) | 18mers_human_m_virus_2MM_722_p.txt | Potato yellow mosaic Panama virus DNA |
| | | A, complete sequence. |
| 176) | 18mers_human_m_virus_2MM_755_p.txt | Sugarcane streak Reunion virus, complete |
| | | genome. |
| 177) | 18mers_human_m_virus_2MM_771_p.txt | Tomato golden mottle virus, complete |
| | | genome. |
| 178) | 18mers_human_m_virus_2MM_784_p.txt | Tomato leaf curl virus, complete genome. |
| 179) | 18mers_human_m_virus_2MM_791_p.txt | Tomato pseudo-curly top virus, complete |
| | | genome. |
| 180) | 18mers_human_m_virus_2MM_816_p.txt | Spiroplasma phage 1-R8A2B, complete |
| | | genome. |
| 181) | 18mers_human_m_virus_2MM_887_p.txt | Goose parvovirus, complete genome. |
| 182) | 18mers_human_m_virus_2MM_929_p.txt | Watermelon spotted wilt virus segment L, |
| | | complete sequence. |
| 183) | 18mers_human_m_virus_2MM_932_p.txt | Australian bat lyssavirus, complete |
| | | genome. |
| 184) | 18mers_human_m_virus_2MM_935_p.txt | Bovine ephemeral fever virus, complete |
| | | genome. |
| 185) | 18mers_human_m_virus_2MM_945_p.txt | Marburg virus, complete genome. |
| 186) | 18mers_human_m_virus_2MM_963_p.txt | Zaire Ebola virus, complete genome. |
| 187) | 18mers_human_m_virus_2MM_1022_p.txt | Avian nephritis virus, complete genome. |
| 188) | 18mers_human_m_virus_2MM_1026_p.txt | Turkey astrovirus, complete genome. |
| 189) | 18mers_human_m_virus_2MM_1176_p.txt | Pestivirus type 3, complete genome. |
| 190) | 18mers_human_m_virus_2MM_1182_p.txt | Yellow fever virus, complete genome. |
| 191) | 18mers_human_m_virus_2MM_1237_p.txt | Ophiostoma mitovirus 3a, complete |
| | | genome. |
| 192) | 18mers_human_m_virus_2MM_1289_p.txt | Human enterovirus D, complete genome. |
| 193) | 18mers_human_m_virus_2MM_1302_p.txt | Cassava common mosaic virus, complete |
| | | genome. |
| 194) | 18mers_human_m_virus_2MM_1315_p.txt | White clover mosaic virus, complete |
| | | genome. |
| 195) | 18mers_human_m_virus_2MM_1326_p.txt | Cowpea aphid-borne mosaic virus, |
| | | complete genome. |
| 196) | 18mers_human_m_virus_2MM_1356_p.txt | Yam mosaic virus, complete genome. |
| 197) | 18mers_human_m_virus_2MM_1361_p.txt | Parsnip yellow fleck virus, complete |
| | | genome. |
| 198) | 18mers_human_m_virus_2MM_1427_p.txt | Japanese iris necrotic ring virus, complete |
| | | genome. |
| 199) | 18mers_human_m_virus_2MM_1444_p.txt | Turnip crinkle virus, complete genome. |
| 200) | 18mers_human_m_virus_2MM_1449_p.txt | Erysimum latent virus, complete genome. |
| 201) | 18mers_human_m_virus_2MM_9_p.txt | Citrus yellow mosaic virus, complete |
| | | genome. |
| 202) | 18mers_human_m_virus_2MM_15_p.txt | Petunia vein clearing virus, complete |
| | | genome. |
| 203) | 18mers_human_m_virus_2MM_17_p.txt | Soybean chlorotic mottle virus, complete |
| | | genome. |
| 204) | 18mers_human_m_virus_2MM_22_p.txt | Arctic ground squirrel hepatitis B virus, |
| | | complete genome. |
| 205) | 18mers_human_m_virus_2MM_37_p.txt | Bovine leukemia virus, complete genome. |
| 206) | 18mers_human_m_virus_2MM_55_p.txt | Moloney murine sarcoma virus, complete |
| | | genome. |
| 207) | 18mers_human_m_virus_2MM_59_p.txt | Murine sarcoma virus, complete genome. |
| 208) | 18mers_human_m_virus_2MM_73_p.txt | Spleen focus-forming virus, complete |
| | | genome. |
| 209) | 18mers_human_m_virus_2MM_77_p.txt | Y73 sarcoma virus, complete genome. |
| 210) | 18mers_human_m_virus_2MM_90_p.txt | Grapevine fanleaf virus satellite RNA, |
| | | complete genome. |
| 211) | 18mers_human_m_virus_2MM_272_p.txt | Saimiriine herpesvirus 2, complete |
| | | genome. |
| 212) | 18mers_human_m_virus_2MM_295_p.txt | Human papillomavirus candHPV85, |
| | | complete genome. |
| 213) | 18mers_human_m_virus_2MM_304_p.txt | Human papillomavirus type 17, complete |
| | | genome. |
| 214) | 18mers_human_m_virus_2MM_305_p.txt | Human papillomavirus type 18, complete |
| | | genome. |
| 215) | 18mers_human_m_virus_2MM_312_p.txt | Human papillomavirus type 24, complete |
| | | genome. |
| 216) | 18mers_human_m_virus_2MM_326_p.txt | Human papillomavirus type 36, complete |
| | | genome. |
| 217) | 18mers_human_m_virus_2MM_327_p.txt | Human papillomavirus type 37, complete |
| | | genome. |
| 218) | 18mers_human_m_virus_2MM_329_p.txt | Human papillomavirus type 39, complete |
| | | genome. |
| 219) | 18mers_human_m_virus_2MM_338_p.txt | Human papillomavirus type 49, complete |
| | | genome. |
| 220) | 18mers_human_m_virus_2MM_346_p.txt | Human papillomavirus type 56, complete |
| | | genome. |
| 221) | 18mers_human_m_virus_2MM_348_p.txt | Human papillomavirus type 58, complete |
| | | genome. |
| 222) | 18mers_human_m_virus_2MM_349_p.txt | Human papillomavirus type 59, complete |
| | | genome. |
| 223) | 18mers_human_m_virus_2MM_415_p.txt | Mycoplasma arthritidis bacteriophage |
| | | MAV1, complete genome. |
| 224) | 18mers_human_m_virus_2MM_422_p.txt | Marine birnavirus segment A, complete |
| | | sequence. |
| 225) | 18mers_human_m_virus_2MM_633_p.txt | Zygosaccharomyces bailii virus Z, |
| | | complete genome. |
| 226) | 18mers_human_m_virus_2MM_639_p.txt | Columbid circovirus, complete genome. |
| 227) | 18mers_human_m_virus_2MM_657_p.txt | Bean golden mosaic virus DNA A, |
| | | complete sequence. |
| 228) | 18mers_human_m_virus_2MM_666_p.txt | Cabbage leaf curl virus DNA A, complete |
| | | sequence. |
| 229) | 18mers_human_m_virus_2MM_685_p.txt | Digitaria streak virus, complete genome. |
| 230) | 18mers_human_m_virus_2MM_700_p.txt | Macroptilium mosaic Puerto Rico virus |
| | | DNA A, complete sequence. |
| 231) | 18mers_human_m_virus_2MM_705_p.txt | Maize streak virus, complete genome. |
| 232) | 18mers_human_m_virus_2MM_711_p.txt | Mungbean yellow mosaic virus DNA A, |
| | | complete sequence. |
| 233) | 18mers_human_m_virus_2MM_714_p.txt | Panicum streak virus, complete genome. |
| 234) | 18mers_human_m_virus_2MM_759_p.txt | Tobacco curly shoot virus, complete |
| | | genome. |
| 235) | 18mers_human_m_virus_2MM_760_p.txt | Tobacco leaf curl Japan virus, complete |
| | | genome. |
| 236) | 18mers_human_m_virus_2MM_761_p.txt | Tobacco leaf curl Kochi virus, complete |
| | | genome. |
| 237) | 18mers_human_m_virus_2MM_769_p.txt | Tomato golden mosaic virus DNA A, |
| | | complete sequence. |
| 238) | 18mers_human_m_virus_2MM_773_p.txt | Tomato leaf curl Bangladesh virus, |
| | | complete genome. |
| 239) | 18mers_human_m_virus_2MM_781_p.txt | Tomato leaf curl Sri Lanka virus, complete |
| | | genome. |
| 240) | 18mers_human_m_virus_2MM_877_p.txt | Aedes albopictus densovirus, complete |
| | | genome. |
| 241) | 18mers_human_m_virus_2MM_886_p.txt | Galleria mellonella densovirus, complete |
| | | genome. |
| 242) | 18mers_human_m_virus_2MM_957_p.txt | Sonchus yellow net virus, complete |
| | | genome. |
| 243) | 18mers_human_m_virus_2MM_960_p.txt | Tupaia paramyxovirus, complete genome. |
| 244) | 18mers_human_m_virus_2MM_976_p.txt | Influenza A virus (A/Puerto |
| | | Rico/8/34/Mount Sinai(HIN1)) segment I, complete |
| 245) | 18mers_human_m_virus_2MM_1124_p.txt | Broad bean wilt virus 2 RNA1, complete |
| | | sequence. |
| 246) | 18mers_human_m_virus_2MM_1140_p.txt | Squash mosaic virus RNA 1, complete |
| | | sequence. |
| 247) | 18mers_human_m_virus_2MM_1158_p.txt | Bovine viral diarrhea virus genotype 2, |
| | | complete genome. |
| 248) | 18mers_human_m_virus_2MM_1172_p.txt | Murray Valley encephalitis virus, complete |
| | | genome. |
| 249) | 18mers_human_m_virus_2MM_1174_p.txt | Pestivirus Reindeer-I, complete genome. |
| 250) | 18mers_human_m_virus_2MM_1288_p.txt | Human enterovirus C, complete genome. |
| 251) | 18mers_human_m_virus_2MM_1303_p.txt | Clover yellow mosaic virus, complete |
| | | genome. |
| 252) | 18mers_human_m_virus_2MM_1308_p.txt | Pepino mosaic virus, complete genome. |
| 253) | 18mers_human_m_virus_2MM_1321_p.txt | Bean common mosaic virus, complete |
| | | genome. |
| 254) | 18mers_human_m_virus_2MM_1327_p.txt | Dasheen mosaic virus, complete genome. |
| 255) | 18mers_human_m_virus_2MM_1338_p.txt | Pepper mottle virus, complete genome. |
| 256) | 18mers_human_m_virus_2MM_1341_p.txt | Potato virus Y, complete genome. |
| 257) | 18mers_human_m_virus_2MM_1343_p.txt | Scallion mosaic virus, complete genome. |
| 258) | 18mers_human_m_virus_2MM_1365_p.txt | Strawberry mottle virus RNA2, complete |
| | | sequence. |
| 259) | 18mers_human_m_virus_2MM_1423_p.txt | Cucumber necrosis virus, complete |
| | | genome. |
| 260) | 18mers_human_m_virus_2MM_1426_p.txt | Hibiscus chlorotic ringspot virus, complete |
| | | genome. |
| 261) | 18mers_human_m_virus_2MM_1428_p.txt | Leek white stripe virus, complete genome. |
| 262) | 18mers_human_m_virus_2MM_1445_p.txt | Apple chlorotic leaf spot virus, complete |
| | | genome. |
| 263) | 18mers_human_m_virus_2MM_1454_p.txt | Carrot mottle mimic virus, complete |
| | | genome. |
| 264) | 18mers_human_m_virus_2MM_30_p.txt | Abelson murine leukemia virus, complete |
| | | genome. |
| 265) | 18mers_human_m_virus_2MM_31_p.txt | Avian carcinoma virus, complete genome. |
| 266) | 18mers_human_m_virus_2MM_46_p.txt | Gibbon ape leukemia virus, complete |
| | | genome. |
| 267) | 18mers_human_m_virus_2MM_56_p.txt | Mouse mammary tumor virus, complete |
| | | genome. |
| 268) | 18mers_human_m_virus_2MM_60_p.txt | Murine type C retrovirus, complete |
| | | genome. |
| 269) | 18mers_human_m_virus_2MM_70_p.txt | Simian T-lymphotropic virus 2, complete |
| | | genome. |
| 270) | 18mers_human_m_virus_2MM_75_p.txt | Walleye dermal sarcoma virus, complete |
| | | genome. |
| 271) | 18mers_human_m_virus_2MM_81_p.txt | Bamboo mosaic virus satellite RNA, |
| | | complete genome. |
| 272) | 18mers_human_m_virus_2MM_169_p.txt | Bacteriophage bIL312, complete genome. |
| 273) | 18mers_human_m_virus_2MM_228_p.txt | Staphylococcus aureus phage phiP68, |
| | | complete genome. |
| 274) | 18mers_human_m_virus_2MM_231_p.txt | Staphylococcus phage 44AHJD, complete |
| | | genome. |
| 275) | 18mers_human_m_virus_2MM_296_p.txt | Human papillomavirus RTRX7, complete |
| | | genome. |
| 276) | 18mers_human_m_virus_2MM_298_p.txt | Human papillomavirus type 11, complete |
| | | genome. |
| 277) | 18mers_human_m_virus_2MM_325_p.txt | Human papillomavirus type 35, complete |
| | | genome. |
| 278) | 18mers_human_m_virus_2MM_333_p.txt | Human papillomavirus type 42, complete |
| | | genome. |
| 279) | 18mers_human_m_virus_2MM_341_p.txt | Human papillomavirus type 51, complete |
| | | genome. |
| 280) | 18mers_human_m_virus_2MM_344_p.txt | Human papillomavirus type 54, complete |
| | | genome. |
| 281) | 18mers_human_m_virus_2MM_351_p.txt | Human papillomavirus type 6, complete |
| | | genome. |
| 282) | 18mers_human_m_virus_2MM_358_p.txt | Human papillomavirus type 69, complete |
| | | genome. |
| 283) | 18mers_human_m_virus_2MM_359_p.txt | Human papillomavirus type 6a, complete |
| | | genome. |
| 284) | 18mers_human_m_virus_2MM_364_p.txt | Human papillomavirus type 74, complete |
| | | genome. |
| 285) | 18mers_human_m_virus_2MM_371_p.txt | Human papillomavirus type 89, complete |
| | | genome. |
| 286) | 18mers_human_m_virus_2MM_379_p.txt | Ovine papillomavirus 2, complete genome. |
| 287) | 18mers_human_m_virus_2MM_396_p.txt | Amsacta moorei entomopoxvirus, complete |
| | | genome. |
| 288) | 18mers_human_m_virus_2MM_416_p.txt | Drosophila x virus segment A, complete |
| | | sequence. |
| 289) | 18mers_human_m_virus_2MM_625_p.txt | Saccharomyces cerevisiae virus L-A, |
| | | complete genome. |
| 290) | 18mers_human_m_virus_2MM_655_p.txt | Bean dwarf mosaic virus DNA A, |
| | | complete sequence. |
| 291) | 18mers_human_m_virus_2MM_668_p.txt | Chayote yellow mosaic virus, complete |
| | | genome. |
| 292) | 18mers_human_m_virus_2MM_692_p.txt | Egyptian sugarcane streak virus, complete |
| | | genome. |
| 293) | 18mers_human_m_virus_2MM_699_p.txt | Ipomoea yellow vein virus, complete |
| | | genome. |
| 294) | 18mers_human_m_virus_2MM_702_p.txt | Macroptilium yellow mosaic Florida virus |
| | | DNA A, complete sequence. |
| 295) | 18mers_human_m_virus_2MM_720_p.txt | Pepper leaf curl Bangladesh virus segment |
| | | A component, complete sequence. |
| 296) | 18mers_human_m_virus_2MM_728_p.txt | Rhynchosia Golden Mosaic Virus |
| | | chromosome DNA A, complete sequence. |
| 297) | 18mers_human_m_virus_2MM_738_p.txt | Sida yellow mosaic virus, complete |
| | | genome. |
| 298) | 18mers_human_m_virus_2MM_785_p.txt | Tomato mosaic Havana virus DNA A, |
| | | complete sequence. |
| 299) | 18mers_human_m_virus_2MM_789_p.txt | Tomato mottle virus DNA A, complete |
| | | sequence. |
| 300) | 18mers_human_m_virus_2MM_825_p.txt | Chlamydia phage PhiCPG1, complete |
| | | genome. |
| 301) | 18mers_human_m_virus_2MM_840_p.txt | Coconut foliar decay virus, complete |
| | | genome. |
| 302) | 18mers_human_m_virus_2MM_923_p.txt | Rift Valley fever virus, complete genome. |
| 303) | 18mers_human_m_virus_2MM_938_p.txt | Hendra virus, complete genome. |
| 304) | 18mers_human_m_virus_2MM_951_p.txt | Rabies virus, complete genome. |
| 305) | 18mers_human_m_virus_2MM_958_p.txt | Spring viremia of carp virus, complete |
| | | genome. |
| 306) | 18mers_human_m_virus_2MM_959_p.txt | Tioman virus, complete genome. |
| 307) | 18mers_human_m_virus_2MM_1120_p.txt | Beet ringspot virus RNA 1, complete |
| | | sequence. |
| 308) | 18mers_human_m_virus_2MM_1177_p.txt | Powassan virus, complete genome. |
| 309) | 18mers_human_m_virus_2MM_1228_p.txt | Pea enation mosaic virus-1, complete |
| | | genome. |
| 310) | 18mers_human_m_virus_2MM_1246_p.txt | Human coronavirus 229E, complete |
| | | genome. |
| 311) | 18mers_human_m_virus_2MM_1277_p.txt | Enterovirus Yanbian 96-83csf, complete |
| | | genome. |
| 312) | 18mers_human_m_virus_2MM_1297_p.txt | Porcine enterovirus B, complete genome. |
| 313) | 18mers_human_m_virus_2MM_1328_p.txt | Japanese yam mosaic virus, complete |
| | | genome. |
| 314) | 18mers_human_m_virus_2MM_1340_p.txt | Potato virus V, complete genome. |
| 315) | 18mers_human_m_virus_2MM_1351_p.txt | Turnip mosaic virus, complete genome. |
| 316) | 18mers_human_m_virus_2MM_1371_p.txt | Southern bean mosaic virus, complete |
| | | genome. |
| 317) | 18mers_human_m_virus_2MM_1373_p.txt | Subterranean clover mottle virus, complete |
| | | genome. |
| 318) | 18mers_human_m_virus_2MM_1374_p.txt | Turnip rosette virus, complete genome. |
| 319) | 18mers_human_m_virus_2MM_1422_p.txt | Cucumber Bulgarian virus, complete |
| | | genome. |
| 320) | 18mers_human_m_virus_2MM_1494_p.txt | Planaria asexual strain-specific virus-like |
| | | element type 1 large DNA |
| 321) | 18mers_human_m_virus_2MM_43_p.txt | Feline leukemia virus, complete genome. |
| 322) | 18mers_human_m_virus_2MM_52_p.txt | Human T-lymphotropic virus 2, complete |
| | | genome. |
| 323) | 18mers_human_m_virus_2MM_67_p.txt | Simian immunodeficiency virus, complete |
| | | genome. |
| 324) | 18mers_human_m_virus_2MM_86_p.txt | Cotton leaf curl virus-associated DNA |
| | | beta, complete genome. |
| 325) | 18mers_human_m_virus_2MM_127_p.txt | Frog adenovirus 1, complete genome. |
| 326) | 18mers_human_m_virus_2MM_281_p.txt | Bovine papillomavirus type 2, complete |
| | | genome. |
| 327) | 18mers_human_m_virus_2MM_282_p.txt | Bovine papillomavirus type 3, complete |
| | | genome. |
| 328) | 18mers_human_m_virus_2MM_291_p.txt | European elk papillomavirus, complete |
| | | genome. |
| 329) | 18mers_human_m_virus_2MM_310_p.txt | Human papillomavirus type 22, complete |
| | | genome. |
| 330) | 18mers_human_m_virus_2MM_322_p.txt | Human papillomavirus type 32, complete |
| | | genome. |
| 331) | 18mers_human_m_virus_2MM_354_p.txt | Human papillomavirus type 63, complete |
| | | genome. |
| 332) | 18mers_human_m_virus_2MM_360_p.txt | Human papillomavirus type 6b, complete |
| | | genome. |
| 333) | 18mers_human_m_virus_2MM_369_p.txt | Human papillomavirus type 86, complete |
| | | genome. |
| 334) | 18mers_human_m_virus_2MM_378_p.txt | Ovine papillomavirus 1, complete genome. |
| 335) | 18mers_human_m_virus_2MM_635_p.txt | Beak and feather disease virus, complete |
| | | genome. |
| 336) | 18mers_human_m_virus_2MM_650_p.txt | Ageratum yellow vein Sri Lanka virus |
| | | segment A, complete sequence. |
| 337) | 18mers_human_m_virus_2MM_669_p.txt | Chilli leaf curl virus, complete genome. |
| 338) | 18mers_human_m_virus_2MM_704_p.txt | Macroptilium yellow mosaic virus, |
| | | complete genome. |
| 339) | 18mers_human_m_virus_2MM_737_p.txt | Sida mottle virus, complete genome. |
| 340) | 18mers_human_m_virus_2MM_776_p.txt | Tomato leaf curl Karnataka virus, complete |
| | | genome. |
| 341) | 18mers_human_m_virus_2MM_804_p.txt | Acholeplasma phage MV-L1, complete |
| | | genome. |
| 342) | 18mers_human_m_virus_2MM_824_p.txt | Chlamydia phage phiCPAR39, complete |
| | | genome. |
| 343) | 18mers_human_m_virus_2MM_871_p.txt | Adeno-associated virus 1, complete |
| | | genome. |
| 344) | 18mers_human_m_virus_2MM_890_p.txt | Junonia coenia densovirus, complete |
| | | genome. |
| 345) | 18mers_human_m_virus_2MM_896_p.txt | Periplaneta fuliginosa densovirus, complete |
| | | genome. |
| 346) | 18mers_human_m_virus_2MM_898_p.txt | Porcine parvovirus, complete genome. |
| 347) | 18mers_human_m_virus_2MM_933_p.txt | Avian paramyxovirus 6, complete genome. |
| 348) | 18mers_human_m_virus_2MM_955_p.txt | Sendai virus, complete genome. |
| 349) | 18mers_human_m_virus_2MM_962_p.txt | Viral hemorrhagic septicemia virus, |
| | | complete genome. |
| 350) | 18mers_human_m_virus_2MM_964_p.txt | Mirafiori lettuce virus RNA 1, complete |
| | | sequence. |
| 351) | 18mers_human_m_virus_2MM_968_p.txt | Influenza A virus RNA segment 1, |
| | | complete sequence. |
| 352) | 18mers_human_m_virus_2MM_1021_p.txt | Shallot virus X, complete genome. |
| 353) | 18mers_human_m_virus_2MM_1095_p.txt | Norwalk virus, complete genome. |
| 354) | 18mers_human_m_virus_2MM_1150_p.txt | Drosophila C virus, complete genome. |
| 355) | 18mers_human_m_virus_2MM_1151_p.txt | Himetobi P virus, complete genome. |
| 356) | 18mers_human_m_virus_2MM_1173_p.txt | Pestivirus Giraffe-1, complete genome. |
| 357) | 18mers_human_m_virus_2MM_1180_p.txt | Tick-borne encephalitis virus, complete |
| | | genome. |
| 358) | 18mers_human_m_virus_2MM_1287_p.txt | Human enterovirus B, complete genome. |
| 359) | 18mers_human_m_virus_2MM_1345_p.txt | Soybean mosaic virus, complete genome. |
| 360) | 18mers_human_m_virus_2MM_1349_p.txt | Tobacco etch virus, complete genome. |
| 361) | 18mers_human_m_virus_2MM_1352_p.txt | Wheat streak mosaic virus, complete |
| | | genome. |
| 362) | 18mers_human_m_virus_2MM_1393_p.txt | Pea early browning virus RNA 1, complete |
| | | sequence. |
| 363) | 18mers_human_m_virus_2MM_1417_p.txt | Carnation Italian ringspot virus, complete |
| | | genome. |
| 364) | 18mers_human_m_virus_2MM_1446_p.txt | Cherry mottle leaf virus, complete genome. |
| 365) | 18mers_human_m_virus_2MM_7_p.txt | Cauliflower mosaic virus, complete |
| | | genome. |
| 366) | 18mers_human_m_virus_2MM_97_p.txt | Panicum mosaic satellite virus, complete |
| | | genome. |
| 367) | 18mers_human_m_virus_2MM_158_p.txt | Bacillus phage GA-1 virion, complete |
| | | genome. |
| 368) | 18mers_human_m_virus_2MM_207_p.txt | Lactococcus lactis bacteriophage u136, |
| | | complete genome. |
| 369) | 18mers_human_m_virus_2MM_317_p.txt | Human papillomavirus type 29, complete |
| | | genome. |
| 370) | 18mers_human_m_virus_2MM_353_p.txt | Human papillomavirus type 61, complete |
| | | genome. |
| 371) | 18mers_human_m_virus_2MM_374_p.txt | Human papillomavirus type 91, complete |
| | | genome. |
| 372) | 18mers_human_m_virus_2MM_377_p.txt | Multimammate rat papillomavirus, |
| | | complete genome. |
| 373) | 18mers_human_m_virus_2MM_382_p.txt | Rabbit oral papillomavirus, complete |
| | | genome. |
| 374) | 18mers_human_m_virus_2MM_383_p.txt | Reindeer papillomavirus, complete |
| | | genome. |
| 375) | 18mers_human_m_virus_2MM_623_p.txt | Leishmania RNA virus 1-4, complete |
| | | genome. |
| 376) | 18mers_human_m_virus_2MM_680_p.txt | Croton yellow vein mosaic virus, complete |
| | | genome. |
| 377) | 18mers_human_m_virus_2MM_729_p.txt | Sida golden mosaic Costa Rica virus DNA |
| | | A, complete sequence. |
| 378) | 18mers_human_m_virus_2MM_736_p.txt | Sida golden yellow vein virus, complete |
| | | genome. |
| 379) | 18mers_human_m_virus_2MM_762_p.txt | Tobacco leaf curl Yamaguchi virus, |
| | | complete genome. |
| 380) | 18mers_human_m_virus_2MM_782_p.txt | Tomato leaf curl Taiwan virus, complete |
| | | genome. |
| 381) | 18mers_human_m_virus_2MM_794_p.txt | Tomato severe leaf curl virus, complete |
| | | genome. |
| 382) | 18mers_human_m_virus_2MM_797_p.txt | Tomato yellow leaf curl Malaga virus, |
| | | complete genome. |
| 383) | 18mers_human_m_virus_2MM_823_p.txt | Chlamydia phage 2 virion, complete |
| | | genome. |
| 384) | 18mers_human_m_virus_2MM_956_p.txt | Snakehead rhabdovirus, complete genome. |
| 385) | 18mers_human_m_virus_2MM_1097_p.txt | Rabbit hemorrhagic disease virus, |
| | | complete genome. |
| 386) | 18mers_human_m_virus_2MM_1134_p.txt | Grapevine fanleaf virus RNA 1, complete |
| | | sequence. |
| 387) | 18mers_human_m_virus_2MM_1199_p.txt | Barley stripe mosaic virus RNA 1, complete |
| | | sequence. |
| 388) | 18mers_human_m_virus_2MM_1206_p.txt | Sacbrood virus, complete genome. |
| 389) | 18mers_human_m_virus_2MM_1268_p.txt | Indian peanut clump virus RNA 1, |
| | | complete sequence. |
| 390) | 18mers_human_m_virus_2MM_1329_p.txt | Johnsongrass mosaic virus, complete |
| | | genome. |
| 391) | 18mers_human_m_virus_2MM_1335_p.txt | Papaya ringspot virus, complete genome. |
| 392) | 18mers_human_m_virus_2MM_1380_p.txt | Cucumber green mottle mosaic virus, |
| | | complete genome. |
| 393) | 18mers_human_m_virus_2MM_1431_p.txt | Oat chlorotic stunt virus, complete |
| | | genome. |
| 394) | 18mers_human_m_virus_2MM_1459_p.txt | Grapevine virus B, complete genome. |
| 395) | 18mers_human_m_virus_2MM_18_p.txt | Strawberry vein banding virus, complete |
| | | genome. |
| 396) | 18mers_human_m_virus_2MM_34_p.txt | Avian sarcoma virus, complete genome. |
| 397) | 18mers_human_m_virus_2MM_106_p.txt | Satellite tobacco mosaic virus, complete |
| | | genome. |
| 398) | 18mers_human_m_virus_2MM_188_p.txt | Bacteriophage ski, complete genome. |
| 399) | 18mers_human_m_virus_2MM_288_p.txt | Deer papillomavirus, complete genome. |
| 400) | 18mers_human_m_virus_2MM_306_p.txt | Human papillomavirus type 19, complete |
| | | genome. |
| 401) | 18mers_human_m_virus_2MM_311_p.txt | Human papillomavirus type 23, complete |
| | | genome. |
| 402) | 18mers_human_m_virus_2MM_355_p.txt | Human papillomavirus type 65, complete |
| | | genome. |
| 403) | 18mers_human_m_virus_2MM_361_p.txt | Human papillomavirus type 7, complete |
| | | genome. |
| 404) | 18mers_human_m_virus_2MM_450_p.txt | Gremmeniella abietina RNA virus MS1 |
| | | RNA 1, complete sequence. |
| 405) | 18mers_human_m_virus_2MM_649_p.txt | Ageratum yellow vein China virus, |
| | | complete genome. |
| 406) | 18mers_human_m_virus_2MM_676_p.txt | Cotton leaf curl Gezira virus, complete |
| | | genome. |
| 407) | 18mers_human_m_virus_2MM_677_p.txt | Cotton leaf curl Kokhran virus, complete |
| | | genome. |
| 408) | 18mers_human_m_virus_2MM_693_p.txt | Eupatorium yellow vein virus, complete |
| | | genome. |
| 409) | 18mers_human_m_virus_2MM_747_p.txt | Squash leaf curl Yunnan virus, complete |
| | | genome. |
| 410) | 18mers_human_m_virus_2MM_764_p.txt | Tobacco leaf curl Zimbabwe virus, |
| | | complete genome. |
| 411) | 18mers_human_m_virus_2MM_772_p.txt | Tomato leaf curl Bangalore virus, complete |
| | | genome. |
| 412) | 18mers_human_m_virus_2MM_792_p.txt | Tomato rugose mosaic virus DNA A, |
| | | complete sequence. |
| 413) | 18mers_human_m_virus_2MM_885_p.txt | Diatraea saccharalis densovirus, complete |
| | | genome. |
| 414) | 18mers_human_m_virus_2MM_937_p.txt | Canine distemper virus, complete genome. |
| 415) | 18mers_human_m_virus_2MM_947_p.txt | Mumps virus, complete genome. |
| 416) | 18mers_human_m_virus_2MM_1070_p.txt | Pelargonium zonate spot virus RNA 1, |
| | | complete sequence. |
| 417) | 18mers_human_m_virus_2MM_1142_p.txt | Tomato black ring virus RNA 1, complete |
| | | sequence. |
| 418) | 18mers_human_m_virus_2MM_1149_p.txt | Cricket paralysis virus, complete genome. |
| 419) | 18mers_human_m_virus_2MM_1156_p.txt | Alkhurma virus, complete genome. |
| 420) | 18mers_human_m_virus_2MM_1169_p.txt | Louping ill virus, complete genome. |
| 421) | 18mers_human_m_virus_2MM_1243_p.txt | Avian infectious bronchitis virus, complete |
| | | genome. |
| 422) | 18mers_human_m_virus_2MM_1244_p.txt | Bovine coronavirus, complete genome. |
| 423) | 18mers_human_m_virus_2MM_1280_p.txt | Equine rhinovirus 3, complete genome. |
| 424) | 18mers_human_m_virus_2MM_1311_p.txt | Potato virus X, complete genome. |
| 425) | 18mers_human_m_virus_2MM_1320_p.txt | Bean common mosaic necrosis virus, |
| | | complete genome. |
| 426) | 18mers_human_m_virus_2MM_1330_p.txt | Leek yellow stripe potyvirus, complete |
| | | genome. |
| 427) | 18mers_human_m_virus_2MM_1346_p.txt | Sugarcane mosaic virus, complete genome. |
| 428) | 18mers_human_m_virus_2MM_1368_p.txt | Lucerne transient streak virus, complete |
| | | genome. |
| 429) | 18mers_human_m_virus_2MM_1370_p.txt | Sesbania mosaic virus, complete genome. |
| 430) | 18mers_human_m_virus_2MM_1383_p.txt | Odontoglossum ringspot virus, complete |
| | | genome. |
| 431) | 18mers_human_m_virus_2MM_1414_p.txt | Artichoke mottled crinkle virus, complete |
| | | genome. |
| 432) | 18mers_human_m_virus_2MM_1451_p.txt | Ononis yellow mosaic virus, complete |
| | | genome. |
| 433) | 18mers_human_m_virus_2MM_104_p.txt | Satellite RNA of Blackcurrant reversion |
| | | virus, complete genome. |
| 434) | 18mers_human_m_virus_2MM_162_p.txt | Bacteriophage B103, complete genome. |
| 435) | 18mers_human_m_virus_2MM_163_p.txt | Bacteriophage bIL170, complete genome. |
| 436) | 18mers_human_m_virus_2MM_165_p.txt | Bacteriophage bIL286, complete genome. |
| 437) | 18mers_human_m_virus_2MM_192_p.txt | Bacteriophage TP901-1, complete genome. |
| 438) | 18mers_human_m_virus_2MM_213_p.txt | Methanothermobacter wolfeii prophage |
| | | psiM100, complete genome. |
| 439) | 18mers_human_m_virus_2MM_232_p.txt | Streptococcus phage Cp-1, complete |
| | | genome. |
| 440) | 18mers_human_m_virus_2MM_289_p.txt | Equinus papillomavirus, complete genome. |
| 441) | 18mers_human_m_virus_2MM_290_p.txt | Equus caballus papillomavirus type 1, |
| | | complete genome. |
| 442) | 18mers_human_m_virus_2MM_380_p.txt | Phocoena spinipinnis papillomavirus, |
| | | complete genome. |
| 443) | 18mers_human_m_virus_2MM_420_p.txt | Infectious pancreatic necrosis virus |
| | | segment A, complete sequence. |
| 444) | 18mers_human_m_virus_2MM_688_p.txt | East African cassava mosaic virus DNA A, |
| | | complete sequence. |
| 445) | 18mers_human_m_virus_2MM_709_p.txt | Mungbean yellow mosaic India virus DNA |
| | | A, complete sequence. |
| 446) | 18mers_human_m_virus_2MM_768_p.txt | Tomato curly stunt virus, complete |
| | | genome. |
| 447) | 18mers_human_m_virus_2MM_795_p.txt | Tomato yellow leaf curl China virus, |
| | | complete genome. |
| 448) | 18mers_human_m_virus_2MM_808_p.txt | Coliphage M13, complete genome. |
| 449) | 18mers_human_m_virus_2MM_876_p.txt | Adeno-associated virus 6, complete |
| | | genome. |
| 450) | 18mers_human_m_virus_2MM_884_p.txt | Casphalia extranea densovirus, complete |
| | | genome. |
| 451) | 18mers_human_m_virus_2MM_1096_p.txt | Porcine enteric calicivirus, complete |
| | | genome. |
| 452) | 18mers_human_m_virus_2MM_1148_p.txt | Black queen cell virus, complete genome. |
| 453) | 18mers_human_m_virus_2MM_1198_p.txt | Hepatitis E virus, complete genome. |
| 454) | 18mers_human_m_virus_2MM_1205_p.txt | Perina nuda picorna-like virus, complete |
| | | genome. |
| 455) | 18mers_human_m_virus_2MM_1225_p.txt | Cereal yellow dwarf virus-RPS, complete |
| | | genome. |
| 456) | 18mers_human_m_virus_2MM_1232_p.txt | Turnip yellows virus, complete genome. |
| 457) | 18mers_human_m_virus_2MM_1249_p.txt | Porcine epidemic diarrhea virus, complete |
| | | genome. |
| 458) | 18mers_human_m_virus_2MM_1295_p.txt | Poliovirus, complete genome. |
| 459) | 18mers_human_m_virus_2MM_1304_p.txt | Cymbidium mosaic virus, complete |
| | | genome. |
| 460) | 18mers_human_m_virus_2MM_1337_p.txt | Peanut mottle virus, complete genome. |
| 461) | 18mers_human_m_virus_2MM_1355_p.txt | Wild potato mosaic virus, complete |
| | | genome. |
| 462) | 18mers_human_m_virus_2MM_1385_p.txt | Pepper mild mottle virus, complete |
| | | genome. |
| 463) | 18mers_human_m_virus_2MM_1418_p.txt | Carnation mottle virus, complete genome. |
| 464) | 18mers_human_m_virus_2MM_1430_p.txt | Melon necrotic spot virus, genome. |
| 465) | 18mers_human_m_virus_2MM_1435_p.txt | Pothos latent virus, complete genome. |
| 466) | 18mers_human_m_virus_2MM_1443_p.txt | Tomato bushy stunt virus, complete |
| | | genome. |
| 467) | 18mers_human_m_virus_2MM_1453_p.txt | Turnip yellow mosaic virus, complete |
| | | genome. |
| 468) | 18mers_human_m_virus_2MM_1460_p.txt | Acyrthosiphon pisum virus, complete |
| | | genome. |
| 469) | 18mers_human_m_virus_2MM_33_p.txt | Avian myelocytomatosis virus, complete |
| | | genome. |
| 470) | 18mers_human_m_virus_2MM_47_p.txt | Human foamy virus, complete genome. |
| 471) | 18mers_human_m_virus_2MM_62_p.txt | Ovine pulmonary adenocarcinoma virus, |
| | | complete genome. |
| 472) | 18mers_human_m_virus_2MM_66_p.txt | Simian foamy virus, complete genome. |
| 473) | 18mers_human_m_virus_2MM_187_p.txt | Bacteriophage r1t, complete genome. |
| 474) | 18mers_human_m_virus_2MM_210_p.txt | Lactococcus phage P335, complete |
| | | genome. |
| 475) | 18mers_human_m_virus_2MM_308_p.txt | Human papillomavirus type 20, complete |
| | | genome. |
| 476) | 18mers_human_m_virus_2MM_318_p.txt | Human papillomavirus type 2a, complete |
| | | genome. |
| 477) | 18mers_human_m_virus_2MM_352_p.txt | Human papillomavirus type 60, complete |
| | | genome. |
| 478) | 18mers_human_m_virus_2MM_438_p.txt | Cryphonectria hypovirus 3, complete |
| | | genome. |
| 479) | 18mers_human_m_virus_2MM_448_p.txt | Fusarium poae virus 1 RNA 1, complete |
| | | sequence. |
| 480) | 18mers_human_m_virus_2MM_673_p.txt | Cotton leaf crumple geminivirus DNA A, |
| | | complete sequence. |
| 481) | 18mers_human_m_virus_2MM_690_p.txt | East African cassava mosaic Zanzibar virus |
| | | DNA A, complete sequence. |
| 482) | 18mers_human_m_virus_2MM_695_p.txt | Honeysuckle yellow vein mosaic virus, |
| | | complete genome. |
| 483) | 18mers_human_m_virus_2MM_706_p.txt | Malvastrum yellow vein virus, complete |
| | | genome. |
| 484) | 18mers_human_m_virus_2MM_716_p.txt | Pepper golden mosaic virus DNA A, |
| | | complete sequence. |
| 485) | 18mers_human_m_virus_2MM_821_p.txt | Vibrio phage VSK, complete genome. |
| 486) | 18mers_human_m_virus_2MM_872_p.txt | Adeno-associated virus 2, complete |
| | | genome. |
| 487) | 18mers_human_m_virus_2MM_882_p.txt | Bovine parvovirus, complete genome. |
| 488) | 18mers_human_m_virus_2MM_1105_p.txt | Hop latent virus, complete genome. |
| 489) | 18mers_human_m_virus_2MM_1130_p.txt | Cycas necrotic stunt virus RNA 1, |
| | | complete sequence. |
| 490) | 18mers_human_m_virus_2MM_1160_p.txt | Deer tick virus, genome. |
| 491) | 18mers_human_m_virus_2MM_1168_p.txt | Langat virus, complete genome. |
| 492) | 18mers_human_m_virus_2MM_1223_p.txt | Beet mild yellowing virus, complete |
| | | genome. |
| 493) | 18mers_human_m_virus_2MM_1306_p.txt | Narcissus mosaic virus, complete genome. |
| 494) | 18mers_human_m_virus_2MM_1307_p.txt | Papaya mosaic virus, complete genome. |
| 495) | 18mers_human_m_virus_2MM_1347_p.txt | Sweet potato feathery mottle virus, |
| | | complete genome. |
| 496) | 18mers_human_m_virus_2MM_1360_p.txt | Maize chlorotic dwarf virus, complete |
| | | genome. |
| 497) | 18mers_human_m_virus_2MM_1419_p.txt | Carnation ringspot virus RNA 1, complete |
| | | sequence. |
| 498) | 18mers_human_m_virus_2MM_1457_p.txt | Tobacco bushy top virus, complete |
| | | genome. |
| 499) | 18mers_human_m_virus_2MM_1458_p.txt | Grapevine virus A, complete genome. |
| 500) | 18mers_human_m_virus_2MM_1493_p.txt | Carrot red leaf luteovirus associated RNA, |
| | | complete genome. |
| 501) | 18mers_human_m_virus_2MM_82_p.txt | Bhendi yellow vein mosaic virus- |
| | | associated DNA beta, complete genome. |
| 502) | 18mers_human_m_virus_2MM_299_p.txt | Human papillomavirus type 12, complete |
| | | genome. |
| 503) | 18mers_human_m_virus_2MM_316_p.txt | Human papillomavirus type 28, complete |
| | | genome. |
| 504) | 18mers_human_m_virus_2MM_631_p.txt | Trichomonas vaginalis virus II, complete |
| | | genome. |
| 505) | 18mers_human_m_virus_2MM_670_p.txt | Chino del tomate virus DNA A, complete |
| | | sequence. |
| 506) | 18mers_human_m_virus_2MM_694_p.txt | Hollyhock leaf crumple virus, complete |
| | | genome. |
| 507) | 18mers_human_m_virus_2MM_743_p.txt | Soybean crinkle leaf virus, complete |
| | | genome. |
| 508) | 18mers_human_m_virus_2MM_754_p.txt | Stachytarpheta leaf curl virus, complete |
| | | genome. |
| 509) | 18mers_human_m_virus_2MM_756_p.txt | Sugarcane streak virus, complete genome. |
| 510) | 18mers_human_m_virus_2MM_799_p.txt | Tomato yellow leaf curl Thailand virus |
| | | DNA A, complete sequence. |
| 511) | 18mers_human_m_virus_2MM_811_p.txt | Enterobacteria phage lke. |
| 512) | 18mers_human_m_virus_2MM_814_p.txt | Pseudomonas phage Pf3, complete |
| | | genome. |
| 513) | 18mers_human_m_virus_2MM_1037_p.txt | Alfalfa mosaic virus RNA 1, complete |
| | | sequence. |
| 514) | 18mers_human_m_virus_2MM_1058_p.txt | Cucumber mosaic virus RNA 1, complete |
| | | sequence. |
| 515) | 18mers_human_m_virus_2MM_1076_p.txt | Spinach latent virus RNA 1, complete |
| | | sequence. |
| 516) | 18mers_human_m_virus_2MM_1165_p.txt | Hepatitis GB virus B, complete genome. |
| 517) | 18mers_human_m_virus_2MM_1221_p.txt | Bean leafroll virus, complete genome. |
| 518) | 18mers_human_m_virus_2MM_1258_p.txt | Epinephelus tauvina nervous necrosis virus |
| | | RNA 1, complete sequence. |
| 519) | 18mers_human_m_virus_2MM_1279_p.txt | Equine rhinitis B virus, complete genome. |
| 520) | 18mers_human_m_virus_2MM_1363_p.txt | Satsuma dwarf virus RNA1, complete |
| | | sequence. |
| 521) | 18mers_human_m_virus_2MM_1387_p.txt | Tobacco mild green mosaic virus, complete |
| | | genome. |
| 522) | 18mers_human_m_virus_2MM_1389_p.txt | Tomato mosaic virus, complete genome. |
| 523) | 18mers_human_m_virus_2MM_1434_p.txt | Pear latent virus, complete genome. |
| 524) | 18mers_human_m_virus_2MM_1436_p.txt | Red clover necrotic mosaic virus RNA 1, |
| | | complete sequence. |
| 525) | 18mers_human_m_virus_2MM_1447_p.txt | Chayote mosaic tymovirus, complete |
| | | genome. |
| 526) | 18mers_human_m_virus_2MM_1461_p.txt | Beet western yellows ST9 associated virus, |
| | | complete genome. |
| 527) | 18mers_human_m_virus_2MM_1496_p.txt | Saccharomyces cerevisiae killer virus MI, |
| | | complete genome. |
| 528) | 18mers_human_m_virus_2MM_1497_p.txt | Sclerophthora macrospora virus B, |
| | | complete genome. |
| 529) | 18mers_human_m_virus_2MM_12_p.txt | Kalanchoe top-spotting virus, complete |
| | | genome. |
| 530) | 18mers_human_m_virus_2MM_45_p.txt | Fujinami sarcoma virus, complete genome. |
| 531) | 18mers_human_m_virus_2MM_57_p.txt | Murine leukemia virus, complete genome. |
| 532) | 18mers_human_m_virus_2MM_168_p.txt | Bacteriophage bIL311, complete genome. |
| 533) | 18mers_human_m_virus_2MM_418_p.txt | Infectious bursal disease virus segment A, |
| | | complete sequence. |
| 534) | 18mers_human_m_virus_2MM_640_p.txt | Goose circovirus, complete genome. |
| 535) | 18mers_human_m_virus_2MM_653_p.txt | Bean calico mosaic virus DNA A, |
| | | complete sequence. |
| 536) | 18mers_human_m_virus_2MM_659_p.txt | Bean golden yellow mosaic virus DNA A, |
| | | complete sequence. |
| 537) | 18mers_human_m_virus_2MM_707_p.txt | Melon chlorotic leaf curl virus, complete |
| | | genome. |
| 538) | 18mers_human_m_virus_2MM_745_p.txt | Squash leaf curl virus A component DNA, |
| | | complete sequence. |
| 539) | 18mers_human_m_virus_2MM_750_p.txt | Squash yellow mild mottle virus DNA A, |
| | | complete sequence. |
| 540) | 18mers_human_m_virus_2MM_774_p.txt | Tomato leaf curl Gujarat virus DNA A, |
| | | complete sequence. |
| 541) | 18mers_human_m_virus_2MM_798_p.txt | Tomato yellow leaf curl Sardinia virus, |
| | | complete genome. |
| 542) | 18mers_human_m_virus_2MM_820_p.txt | Vibrio cholerae phage VGJphi virion, |
| | | complete genome. |
| 543) | 18mers_human_m_virus_2MM_880_p.txt | Bombyx mori densovirus 1, complete |
| | | genome. |
| 544) | 18mers_human_m_virus_2MM_946_p.txt | Measles virus, complete genome. |
| 545) | 18mers_human_m_virus_2MM_1020_p.txt | Garlic virus X, complete genome. |
| 546) | 18mers_human_m_virus_2MM_1025_p.txt | Ovine astrovirus, complete genome. |
| 547) | 18mers_human_m_virus_2MM_1113_p.txt | Lettuce infectious yellows virus RNA 1, |
| | | complete sequence. |
| 548) | 18mers_human_m_virus_2MM_1181_p.txt | West Nile virus, complete genome. |
| 549) | 18mers_human_m_virus_2MM_1207_p.txt | Bacteriophage AP205 virion, complete |
| | | genome. |
| 550) | 18mers_human_m_virus_2MM_1217_p.txt | Barley yellow dwarf virus - GAV, |
| | | complete genome. |
| 551) | 18mers_human_m_virus_2MM_1218_p.txt | Barley yellow dwarf virus - MAV, |
| | | complete genome. |
| 552) | 18mers_human_m_virus_2MM_1220_p.txt | Barley yellow dwarf virus-PAS, complete |
| | | genome. |
| 553) | 18mers_human_m_virus_2MM_1222_p.txt | Beet chlorosis virus, complete genome. |
| 554) | 18mers_human_m_virus_2MM_1224_p.txt | Beet western yellows virus, complete |
| | | genome. |
| 555) | 18mers_human_m_virus_2MM_1247_p.txt | Lactate dehydrogenase-elevating virus, |
| | | complete genome. |
| 556) | 18mers_human_m_virus_2MM_1312_p.txt | Scallion virus X, complete genome. |
| 557) | 18mers_human_m_virus_2MM_1313_p.txt | Strawberry mild yellow edge virus, |
| | | complete genome. |
| 558) | 18mers_human_m_virus_2MM_1357_p.txt | Zucchini yellow mosaic virus, complete |
| | | genome. |
| 559) | 18mers_human_m_virus_2MM_1432_p.txt | Olive latent virus 1, complete genome. |
| 560) | 18mers_human_m_virus_2MM_1438_p.txt | Saguaro cactus virus, complete genome. |
| 561) | 18mers_human_m_virus_2MM_841_p.txt | Faba bean necrotic yellows virus DNA 1, |
| | | complete sequence. |
| 562) | 18mers_human_m_virus_2MM_32_p.txt | Avian leukosis virus, complete genome. |
| 563) | 18mers_human_m_virus_2MM_212_p.txt | Methanobacterium phage psiM2, complete |
| | | genome. |
| 564) | 18mers_human_m_virus_2MM_307_p.txt | Human papillomavirus type 1a, complete |
| | | genome. |
| 565) | 18mers_human_m_virus_2MM_319_p.txt | Human papillomavirus type 3, complete |
| | | genome. |
| 566) | 18mers_human_m_virus_2MM_331_p.txt | Human papillomavirus type 40, complete |
| | | genome. |
| 567) | 18mers_human_m_virus_2MM_367_p.txt | Human papillomavirus type 83, complete |
| | | genome. |
| 568) | 18mers_human_m_virus_2MM_407_p.txt | Sheeppox virus, complete genome. |
| 569) | 18mers_human_m_virus_2MM_718_p.txt | Pepper huasteco yellow vein virus DNA A, |
| | | complete sequence. |
| 570) | 18mers_human_m_virus_2MM_807_p.txt | Bacteriophage VSKK, complete genome. |
| 571) | 18mers_human_m_virus_2MM_834_p.txt | Banana bunchy top virus DNA 1, complete |
| | | sequence. |
| 572) | 18mers_human_m_virus_2MM_1046_p.txt | Broad bean mottle virus RNA 1, complete |
| | | sequence. |
| 573) | 18mers_human_m_virus_2MM_1167_p.txt | Japanese encephalitis virus, genome. |
| 574) | 18mers_human_m_virus_2MM_1211_p.txt | Enterobacteria phage GA, complete |
| | | genome. |
| 575) | 18mers_human_m_virus_2MM_1301_p.txt | Bamboo mosaic virus, complete genome. |
| 576) | 18mers_human_m_virus_2MM_1464_p.txt | Euprosterna elaeasa virus, complete |
| | | genome. |
| 577) | 18mers_human_m_virus_2MM_1489_p.txt | Streptococcus pyogenes phage 315.3 |
| | | provirus, complete genome. |
| 578) | 18mers_human_m_virus_2MM_167_p.txt | Bacteriophage bIL310, complete genome. |
| 579) | 18mers_human_m_virus_2MM_193_p.txt | Bacteriophage Tuc2009, complete genome. |
| 580) | 18mers_human_m_virus_2MM_208_p.txt | Lactococcus phage BK5-T, complete |
| | | genome. |
| 581) | 18mers_human_m_virus_2MM_248_p.txt | Ateline herpesvirus 3, complete genome. |
| 582) | 18mers_human_m_virus_2MM_313_p.txt | Human papillomavirus type 25, complete |
| | | genome. |
| 583) | 18mers_human_m_virus_2MM_679_p.txt | Cotton leaf curl Rajasthan virus segment |
| | | A, complete sequence. |
| 584) | 18mers_human_m_virus_2MM_726_p.txt | Potato yellow mosaic virus DNA A, |
| | | complete sequence. |
| 585) | 18mers_human_m_virus_2MM_862_p.txt | Subterranean clover stunt virus DNA 1, |
| | | complete sequence. |
| 586) | 18mers_human_m_virus_2MM_873_p.txt | Adeno-associated virus 3, complete |
| | | genome. |
| 587) | 18mers_human_m_virus_2MM_874_p.txt | Adeno-associated virus 3B, complete |
| | | genome. |
| 588) | 18mers_human_m_virus_2MM_881_p.txt | Bombyx mori densovirus 5, complete |
| | | genome. |
| 589) | 18mers_human_m_virus_2MM_893_p.txt | Minute virus of canines, genome. |
| 590) | 18mers_human_m_virus_2MM_934_p.txt | Boma disease virus, complete genome. |
| 591) | 18mers_human_m_virus_2MM_948_p.txt | Newcastle disease virus, complete genome. |
| 592) | 18mers_human_m_virus_2MM_1098_p.txt | Vesicular exanthema of swine virus, |
| | | complete genome. |
| 593) | 18mers_human_m_virus_2MM_1194_p.txt | Soil-borne wheat mosaic virus RNA 1, |
| | | complete sequence. |
| 594) | 18mers_human_m_virus_2MM_1229_p.txt | Potato leafroll virus, complete genome. |
| 595) | 18mers_human_m_virus_2MM_1332_p.txt | Maize dwarf mosaic virus, complete |
| | | genome. |
| 596) | 18mers_human_m_virus_2MM_1469_p.txt | Bacteriophage IN93, complete genome. |
| 597) | 18mers_human_m_virus_2MM_51_p.txt | Human T-lymphotropic virus 1, complete |
| | | genome. |
| 598) | 18mers_human_m_virus_2MM_297_p.txt | Human papillomavirus type 10, complete |
| | | genome. |
| 599) | 18mers_human_m_virus_2MM_446_p.txt | Discula destructiva virus 2 segment 1, |
| | | complete sequence. |
| 600) | 18mers_human_m_virus_2MM_575_p.txt | Rice black streaked dwarf virus segment 1, |
| | | complete sequence. |
| 601) | 18mers_human_m_virus_2MM_638_p.txt | Chicken anemia virus, complete genome. |
| 602) | 18mers_human_m_virus_2MM_741_p.txt | South African cassava mosaic virus DNA |
| | | A, complete sequence. |
| 603) | 18mers_human_m_virus_2MM_819_p.txt | Vibrio cholerae O139 fs1 phage, complete |
| | | genome. |
| 604) | 18mers_human_m_virus_2MM_870_p.txt | Acheta domesticus densovirus, complete |
| | | genome. |
| 605) | 18mers_human_m_virus_2MM_953_p.txt | Reston Ebola virus, complete genome. |
| 606) | 18mers_human_m_virus_2MM_1024_p.txt | Mink astrovirus, complete genome. |
| 607) | 18mers_human_m_virus_2MM_1067_p.txt | Peanut stunt virus RNA 1, complete |
| | | sequence. |
| 608) | 18mers_human_m_virus_2MM_1093_p.txt | European brown hare syndrome virus, |
| | | complete genome. |
| 609) | 18mers_human_m_virus_2MM_1116_p.txt | Sweet potato chlorotic stunt virus RNA 1, |
| | | complete sequence. |
| 610) | 18mers_human_m_virus_2MM_1146_p.txt | Acute bee paralysis virus, complete |
| | | genome. |
| 611) | 18mers_human_m_virus_2MM_1219_p.txt | Barley yellow dwarf virus - PAV, complete |
| | | genome. |
| 612) | 18mers_human_m_virus_2MM_1251_p.txt | SARS coronavirus, complete genome. |
| 613) | 18mers_human_m_virus_2MM_1372_p.txt | Southern cowpea mosaic virus, complete |
| | | genome. |
| 614) | 18mers_human_m_virus_2MM_1424_p.txt | Cymbidium ringspot virus, complete |
| | | genome. |
| 615) | 18mers_human_m_virus_2MM_1439_p.txt | Sweet clover necrotic mosaic virus RNA 1, |
| | | complete sequence. |
| 616) | 18mers_human_m_virus_2MM_1455_p.txt | Groundnut rosette virus, complete genome. |
| 617) | 18mers_human_m_virus_2MM_283_p.txt | Bovine papillomavirus type 4, complete |
| | | genome. |
| 618) | 18mers_human_m_virus_2MM_453_p.txt | Mycovirus FusoV RNA 1, complete |
| | | sequence. |
| 619) | 18mers_human_m_virus_2MM_675_p.txt | Cotton leaf curl Alabad virus, complete |
| | | genome. |
| 620) | 18mers_human_m_virus_2MM_724_p.txt | Potato yellow mosaic Trinidad virus DNA |
| | | A, complete sequence. |
| 621) | 18mers_human_m_virus_2MM_744_p.txt | Squash leaf curl China virus, complete |
| | | genome. |
| 622) | 18mers_human_m_virus_2MM_827_p.txt | Coliphage alpha3, complete genome. |
| 623) | 18mers_human_m_virus_2MM_1027_p.txt | Mushroom bacilliform virus, complete |
| | | genome. |
| 624) | 18mers_human_m_virus_2MM_1028_p.txt | Beet necrotic yellow vein virus RNA 1, |
| | | complete sequence. |
| 625) | 18mers_human_m_virus_2MM_1106_p.txt | Potato virus M, complete genome. |
| 626) | 18mers_human_m_virus_2MM_1227_p.txt | Cucurbit aphid-borne yellows virus, |
| | | complete genome. |
| 627) | 18mers_human_m_virus_2MM_1230_p.txt | Soybean dwarf virus, complete genome. |
| 628) | 18mers_human_m_virus_2MM_1235_p.txt | Poinsettia mosaic virus, complete genome. |
| 629) | 18mers_human_m_virus_2MM_1248_p.txt | Murine hepatitis virus, complete genome. |
| 630) | 18mers_human_m_virus_2MM_1344_p.txt | Sorghum mosaic virus, complete genome. |
| 631) | 18mers_human_m_virus_2MM_1397_p.txt | Tobacco rattle virus RNA 1, complete |
| | | sequence. |
| 632) | 18mers_human_m_virus_2MM_1421_p.txt | Cowpea mottle virus, complete genome. |
| 633) | 18mers_human_m_virus_2MM_1425_p.txt | Galinsoga mosaic virus, complete genome. |
| 634) | 18mers_human_m_virus_2MM_1487_p.txt | Streptococcus pyogenes phage 315.1 |
| | | provirus, complete genome. |
| 635) | 18mers_human_m_virus_2MM_166_p.txt | Bacteriophage bIL309, complete genome. |
| 636) | 18mers_human_m_virus_2MM_209_p.txt | Lactococcus phage c2. |
| 637) | 18mers_human_m_virus_2MM_293_p.txt | Fringilla coelebs papillomavirus, complete |
| | | genome. |
| 638) | 18mers_human_m_virus_2MM_442_p.txt | Discula destructiva virus 1 segment 1, |
| | | complete sequence. |
| 639) | 18mers_human_m_virus_2MM_624_p.txt | Leishmania RNA virus 2-1, complete |
| | | genome. |
| 640) | 18mers_human_m_virus_2MM_629_p.txt | Trichomonas vaginalis virus, complete |
| | | genome. |
| 641) | 18mers_human_m_virus_2MM_739_p.txt | Sida yellow vein virus DNA A, complete |
| | | sequence. |
| 642) | 18mers_human_m_virus_2MM_829_p.txt | Enterobacteria phage G4, complete |
| | | genome. |
| 643) | 18mers_human_m_virus_2MM_1092_p.txt | Canine calicivirus, complete genome. |
| 644) | 18mers_human_m_virus_2MM_1122_p.txt | Blackcurrant reversion virus RNA1, |
| | | complete sequence. |
| 645) | 18mers_human_m_virus_2MM_1272_p.txt | Aichi virus, complete genome. |
| 646) | 18mers_human_m_virus_2MM_1309_p.txt | Plantago asiatica mosaic virus, complete |
| | | genome. |
| 647) | 18mers_human_m_virus_2MM_1333_p.txt | Oat mosaic virus RNA 1, complete |
| | | sequence. |
| 648) | 18mers_human_m_virus_2MM_1367_p.txt | Cocksfoot mottle virus, complete genome. |
| 649) | 18mers_human_m_virus_2MM_1378_p.txt | Crucifer tobamovirus, complete genome. |
| 650) | 18mers_human_m_virus_2MM_1386_p.txt | Ribgrass mosaic virus, complete genome. |
| 651) | 18mers_human_m_virus_2MM_1429_p.txt | Maize chlorotic mottle virus, complete |
| | | genome. |
| 652) | 18mers_human_m_virus_2MM_1462_p.txt | Botrytis virus F, complete genome. |
| 653) | 18mers_human_m_virus_2MM_69_p.txt | Simian T-lymphotropic virus 1, complete |
| | | genome. |
| 654) | 18mers_human_m_virus_2MM_301_p.txt | Human papillomavirus type 14D, complete |
| | | genome. |
| 655) | 18mers_human_m_virus_2MM_347_p.txt | Human papillomavirus type 57, complete |
| | | genome. |
| 656) | 18mers_human_m_virus_2MM_622_p.txt | Leishmania RNA virus 1-1, complete |
| | | genome. |
| 657) | 18mers_human_m_virus_2MM_686_p.txt | East African cassava mosaic Cameroon |
| | | virus DNA A, complete sequence. |
| 658) | 18mers_human_m_virus_2MM_734_p.txt | Sida golden mosaic virus DNA-A, |
| | | complete sequence. |
| 659) | 18mers_human_m_virus_2MM_1055_p.txt | Cowpea chlorotic mottle virus RNA 1, |
| | | complete sequence. |
| 660) | 18mers_human_m_virus_2MM_1073_p.txt | Prunus necrotic ringspot virus RNA 1, |
| | | complete sequence. |
| 661) | 18mers_human_m_virus_2MM_1115_p.txt | Little cherry virus 1, complete genome. |
| 662) | 18mers_human_m_virus_2MM_1202_p.txt | Raspberry bushy dwarf virus RNA 1, |
| | | complete sequence. |
| 663) | 18mers_human_m_virus_2MM_1226_p.txt | Cereal yellow dwarf virus-RPV, complete |
| | | genome. |
| 664) | 18mers_human_m_virus_2MM_1231_p.txt | Sugarcane yellow leaf virus, complete |
| | | genome. |
| 665) | 18mers_human_m_virus_2MM_1331_p.txt | Lettuce mosaic virus, complete genome. |
| 666) | 18mers_human_m_virus_2MM_1348_p.txt | Sweet potato mild mottle virus, complete |
| | | genome. |
| 667) | 18mers_human_m_virus_2MM_1391_p.txt | Youcai mosaic virus, complete genome. |
| 668) | 18mers_human_m_virus_2MM_1412_p.txt | Venezuelan equine encephalitis virus, |
| | | complete genome. |
| 669) | 18mers_human_m_virus_2MM_65_p.txt | Rous sarcoma virus, complete genome. |
| 670) | 18mers_human_m_virus_2MM_123_p.txt | Canine adenovirus type 1, complete |
| | | genome. |
| 671) | 18mers_human_m_virus_2MM_159_p.txt | Bacillus phage PZA, complete genome. |
| 672) | 18mers_human_m_virus_2MM_229_p.txt | Staphylococcus aureus prophage phiPV83 |
| | | provirus, complete genome. |
| 673) | 18mers_human_m_virus_2MM_315_p.txt | Human papillomavirus type 27, complete |
| | | genome. |
| 674) | 18mers_human_m_virus_2MM_401_p.txt | Goatpox virus, complete genome. |
| 675) | 18mers_human_m_virus_2MM_630_p.txt | Trichomonas vaginalis virus 3, complete |
| | | genome. |
| 676) | 18mers_human_m_virus_2MM_665_p.txt | Bhendi yellow vein mosaic virus, complete |
| | | genome. |
| 677) | 18mers_human_m_virus_2MM_678_p.txt | Cotton leaf curl Multan virus, complete |
| | | genome. |
| 678) | 18mers_human_m_virus_2MM_779_p.txt | Tomato leaf curl New Delhi virus DNA A, |
| | | complete sequence. |
| 679) | 18mers_human_m_virus_2MM_851_p.txt | Milk vetch dwarf virus segment 1, |
| | | complete sequence. |
| 680) | 18mers_human_m_virus_2MM_1099_p.txt | Walrus calicivirus, complete genome. |
| 681) | 18mers_human_m_virus_2MM_1104_p.txt | Garlic latent virus, complete genome. |
| 682) | 18mers_human_m_virus_2MM_1274_p.txt | Bovine enterovirus, complete genome. |
| 683) | 18mers_human_m_virus_2MM_1318_p.txt | Barley yellow mosaic virus RNA 1, |
| | | complete sequence. |
| 684) | 18mers_human_m_virus_2MM_1379_p.txt | Cucumber fruit mottle mosaic virus, |
| | | complete genome. |
| 685) | 18mers_human_m_virus_2MM_1400_p.txt | Barmah Forest virus, complete genome. |
| 686) | 18mers_human_m_virus_2MM_1441_p.txt | Tobacco necrosis virus A, complete |
| | | genome. |
| 687) | 18mers_human_m_virus_2MM_1484_p.txt | Staphylococcus aureus phage phi 12 |
| | | provirus, complete genome. |
| 688) | 18mers_human_m_virus_2MM_205_p.txt | Lactobacillus bacteriophage phi adh, |
| | | complete genome. |
| 689) | 18mers_human_m_virus_2MM_223_p.txt | Roseophage SIO1, complete genome. |
| 690) | 18mers_human_m_virus_2MM_246_p.txt | Sulfolobus virus 1, complete genome. |
| 691) | 18mers_human_m_virus_2MM_787_p.txt | Tomato mottle Taino virus DNA A, |
| | | complete sequence. |
| 692) | 18mers_human_m_virus_2MM_809_p.txt | Enterobacteria phage 12-2. |
| 693) | 18mers_human_m_virus_2MM_810_p.txt | Enterobacteria phage 1f1, complete |
| | | genome. |
| 694) | 18mers_human_m_virus_2MM_831_p.txt | Phage phiMH2K virion, complete genome. |
| 695) | 18mers_human_m_virus_2MM_1033_p.txt | Beet soil-borne mosaic virus RNA1, |
| | | complete sequence. |
| 696) | 18mers_human_m_virus_2MM_1085_p.txt | Tomato aspermy virus RNA 1, complete |
| | | sequence. |
| 697) | 18mers_human_m_virus_2MM_1204_p.txt | Infectious flacherie virus, complete |
| | | genome. |
| 698) | 18mers_human_m_virus_2MM_1234_p.txt | Oat blue dwarf virus, complete genome. |
| 699) | 18mers_human_m_virus_2MM_1260_p.txt | Flock house virus, complete genome. |
| 700) | 18mers_human_m_virus_2MM_1305_p.txt | Foxtail mosaic virus, complete genome. |
| 701) | 18mers_human_m_virus_2MM_1362_p.txt | Rice tungro spherical virus, complete |
| | | genome. |
| 702) | 18mers_human_m_virus_2MM_644_p.txt | Abutilon mosaic virus A component DNA, |
| | | complete sequence. |
| 703) | 18mers_human_m_virus_2MM_713_p.txt | Okra yellow vein mosaic virus, complete |
| | | genome. |
| 704) | 18mers_human_m_virus_2MM_802_p.txt | Watermelon chlorotic stunt virus DNA A, |
| | | complete sequence. |
| 705) | 18mers_human_m_virus_2MM_830_p.txt | Enterobacteria phage S13, complete |
| | | genome. |
| 706) | 18mers_human_m_virus_2MM_1043_p.txt | Apple mosaic virus RNA 1, complete |
| | | sequence. |
| 707) | 18mers_human_m_virus_2MM_1270_p.txt | Peanut clump virus RNA 1, complete |
| | | sequence. |
| 708) | 18mers_human_m_virus_2MM_1278_p.txt | Equine rhinitis A virus, complete genome. |
| 709) | 18mers_human_m_virus_2MM_1384_p.txt | Paprika mild mottle virus, complete |
| | | genome. |
| 710) | 18mers_human_m_virus_2MM_1395_p.txt | Pepper ringspot virus RNA 1, complete |
| | | sequence. |
| 711) | 18mers_human_m_virus_2MM_1465_p.txt | Grapevine fleck virus, complete genome. |
| 712) | 18mers_human_m_virus_2MM_1485_p.txt | Staphylococcus aureus phage phi 13 |
| | | provirus, complete genome. |
| 713) | 18mers_human_m_virus_2MM_697_p.txt | Indian cassava mosaic virus DNA B, |
| | | complete sequence. |
| 714) | 18mers_human_m_virus_2MM_752_p.txt | Sri Lankan cassava mosaic virus DNA A, |
| | | complete sequence. |
| 715) | 18mers_human_m_virus_2MM_1061_p.txt | Elm mottle virus RNA 1, complete |
| | | sequence. |
| 716) | 18mers_human_m_virus_2MM_1154_p.txt | Taura syndrome virus, complete genome. |
| 717) | 18mers_human_m_virus_2MM_1264_p.txt | Pariacoto virus RNA1, complete sequence. |
| 718) | 18mers_human_m_virus_2MM_1390_p.txt | Turnip vein-clearing virus, complete |
| | | genome. |
| 719) | 18mers_human_m_virus_2MM_211_p.txt | Listeria phage 2389 virion, complete |
| | | genome. |
| 720) | 18mers_human_m_virus_2MM_455_p.txt | Rhizoctonia solani virus RNA 1, complete |
| | | sequence. |
| 721) | 18mers_human_m_virus_2MM_632_p.txt | Ustilago maydis virus H1, complete |
| | | genome. |
| 722) | 18mers_human_m_virus_2MM_681_p.txt | Cucurbit leaf curl virus DNA A, complete |
| | | sequence. |
| 723) | 18mers_human_m_virus_2MM_1103_p.txt | Blueberry scorch virus, complete genome. |
| 724) | 18mers_human_m_virus_2MM_1233_p.txt | Maize rayado fino virus, complete genome. |
| 725) | 18mers_human_m_virus_2MM_1353_p.txt | Wheat yellow mosaic virus RNA 1, |
| | | complete sequence. |
| 726) | 18mers_human_m_virus_2MM_1382_p.txt | Obuda pepper virus, complete genome. |
| 727) | 18mers_human_m_virus_2MM_1388_p.txt | Tobacco mosaic virus, complete genome. |
| 728) | 18mers_human_m_virus_2MM_1456_p.txt | Pea enation mosaic virus-2, complete |
| | | genome. |
| 729) | 18mers_human_m_virus_2MM_275_p.txt | Invertebrate iridescent virus 6, complete |
| | | genome. |
| 730) | 18mers_human_m_virus_2MM_391_p.txt | Budgerigar fledgling polyomavirus, |
| | | complete genome. |
| 731) | 18mers_human_m_virus_2MM_402_p.txt | Lumpy skin disease virus, complete |
| | | genome. |
| 732) | 18mers_human_m_virus_2MM_620_p.txt | Gremmeniella abietina RNA virus L1, |
| | | complete genome. |
| 733) | 18mers_human_m_virus_2MM_732_p.txt | Sida golden mosaic Honduras virus DNA |
| | | A, complete sequence. |
| 734) | 18mers_human_m_virus_2MM_818_p.txt | Vibrio cholerae filamentous bacteriophage |
| | | fs-2, complete genome. |
| 735) | 18mers_human_m_virus_2MM_1164_p.txt | Hepatitis GB virus A, complete genome. |
| 736) | 18mers_human_m_virus_2MM_1188_p.txt | Chinese wheat mosaic virus RNA 1, |
| | | complete sequence. |
| 737) | 18mers_human_m_virus_2MM_1192_p.txt | Soil-borne cereal mosaic virus RNA 1, |
| | | complete sequence. |
| 738) | 18mers_human_m_virus_2MM_1212_p.txt | Enterobacteria phage KU1, complete |
| | | genome. |
| 739) | 18mers_human_m_virus_2MM_1275_p.txt | Bovine kobuvirus, complete genome. |
| 740) | 18mers_human_m_virus_2MM_1323_p.txt | Brome streak mosaic virus, complete |
| | | genome. |
| 741) | 18mers_human_m_virus_2MM_1088_p.txt | Tulare apple mosaic virus RNA1, complete |
| | | sequence. |
| 742) | 18mers_human_m_virus_2MM_1282_p.txt | Foot-and-mouth disease virus O, complete |
| | | genome. |
| 743) | 18mers_human_m_virus_2MM_1316_p.txt | Barley mild mosaic virus RNA 1, complete |
| | | sequence. |
| 744) | 18mers_human_m_virus_2MM_1404_p.txt | Mayaro virus, complete genome. |
| 745) | 18mers_human_m_virus_2MM_1416_p.txt | Cardamine chlorotic fleck virus, complete |
| | | genome. |
| 746) | 18mers_human_m_virus_2MM_565_p.txt | Nilaparvata lugens reovirus segment 1, |
| | | complete sequence. |
| 747) | 18mers_human_m_virus_2MM_619_p.txt | Giardia lamblia virus, complete genome. |
| 748) | 18mers_human_m_virus_2MM_828_p.txt | Coliphage phiX174, complete genome. |
| 749) | 18mers_human_m_virus_2MM_1091_p.txt | Calicivirus strain NB, complete genome. |
| 750) | 18mers_human_m_virus_2MM_237_p.txt | Streptococcus thermophilus bacteriophage |
| | | Sfi21, complete genome. |
| 751) | 18mers_human_m_virus_2MM_897_p.txt | Planococcus citri densovirus, complete |
| | | genome. |
| 752) | 18mers_human_m_virus_2MM_1018_p.txt | Garlic virus C, complete genome. |
| 753) | 18mers_human_m_virus_2MM_1052_p.txt | Citrus leaf rugose virus RNA 1, complete |
| | | sequence. |
| 754) | 18mers_human_m_virus_2MM_1162_p.txt | Hepatitis C virus, complete genome. |
| 755) | 18mers_human_m_virus_2MM_1213_p.txt | Enterobacteria phage MX1. |
| 756) | 18mers_human_m_virus_2MM_1242_p.txt | Saccharomyces cerevisiae narnavirus 23S |
| | | RNA &lt;T&gt;, complete genome. |
| 757) | 18mers_human_m_virus_2MM_1266_p.txt | Striped Jack nervous necrosis virus RNA1, |
| | | complete sequence. |
| 758) | 18mers_human_m_virus_2MM_1403_p.txt | Igbo Ora virus, complete genome. |
| 759) | 18mers_human_m_virus_2MM_1082_p.txt | Tobacco streak virus RNA 1, complete |
| | | sequence. |
| 760) | 18mers_human_m_virus_2MM_1144_p.txt | Tomato ringspot virus RNA 1, complete |
| | | sequence. |
| 761) | 18mers_human_m_virus_2MM_1254_p.txt | Black beetle virus RNA 1, complete |
| | | sequence. |
| 762) | 18mers_human_m_virus_2MM_1300_p.txt | Theilovirus, complete genome. |
| 763) | 18mers_human_m_virus_2MM_1314_p.txt | Tulip virus X, complete genome. |
| 764) | 18mers_human_m_virus_2MM_1392_p.txt | Zucchini green mottle mosaic virus, |
| | | complete genome. |
| 765) | 18mers_human_m_virus_2MM_179_p.txt | Bacteriophage phi ETA, complete genome. |
| 766) | 18mers_human_m_virus_2MM_230_p.txt | Staphylococcus aureus temperate phage |
| | | phiSLT virion, complete genome. |
| 767) | 18mers_human_m_virus_2MM_234_p.txt | Streptococcus thermophilus bacteriophage |
| | | DT1, complete genome. |
| 768) | 18mers_human_m_virus_2MM_270_p.txt | Murid herpesvirus 4, complete genome. |
| 769) | 18mers_human_m_virus_2MM_748_p.txt | Squash mild leaf curl virus DNA A, |
| | | complete sequence. |
| 770) | 18mers_human_m_virus_2MM_1166_p.txt | Hepatitis GB virus C, complete genome. |
| 771) | 18mers_human_m_virus_2MM_1208_p.txt | Bacteriophage M11, complete genome. |
| 772) | 18mers_human_m_virus_2MM_1281_p.txt | Foot-and-mouth disease virus C, complete |
| | | genome. |
| 773) | 18mers_human_m_virus_2MM_1467_p.txt | Oyster mushroom spherical virus, complete |
| | | genome. |
| 774) | 18mers_human_m_virus_2MM_381_p.txt | Psittacus erithacus papillomavirus, |
| | | complete genome. |
| 775) | 18mers_human_m_virus_2MM_457_p.txt | Banna virus segment 1, complete sequence. |
| 776) | 18mers_human_m_virus_2MM_1147_p.txt | Aphid lethal paralysis virus, complete |
| | | genome. |
| 777) | 18mers_human_m_virus_2MM_1159_p.txt | Cell fusing agent virus, complete genome. |
| 778) | 18mers_human_m_virus_2MM_189_p.txt | Bacteriophage SPBc2, complete genome. |
| 779) | 18mers_human_m_virus_2MM_1109_p.txt | Cucumber yellows virus RNA 1, complete |
| | | sequence. |
| 780) | 18mers_human_m_virus_2MM_1256_p.txt | Boolarra virus, complete genome. |
| 781) | 18mers_human_m_virus_2MM_1369_p.txt | Rice yellow mottle virus, complete |
| | | genome. |
| 782) | 18mers_human_m_virus_2MM_618_p.txt | Eimeria brunetti RNA virus 1, complete |
| | | genome. |
| 783) | 18mers_human_m_virus_2MM_1190_p.txt | Oat golden stripe virus RNA 1, complete |
| | | sequence. |
| 784) | 18mers_human_m_virus_2MM_1466_p.txt | Indian citrus ringspot virus, complete |
| | | genome. |
| 785) | 18mers_human_m_virus_2MM_1413_p.txt | Western equine encephalomyelitis virus, |
| | | complete genome. |
| 786) | 18mers_human_m_virus_2MM_227_p.txt | Staphylococcus aureus bacteriophage PVL |
| | | provirus, complete genome. |
| 787) | 18mers_human_m_virus_2MM_238_p.txt | Streptococcus thermophilus temperate |
| | | bacteriophage O1205, complete genome. |
| 788) | 18mers_human_m_virus_2MM_247_p.txt | Alcelaphine herpesvirus 1, complete |
| | | genome. |
| 789) | 18mers_human_m_virus_2MM_1064_p.txt | Olive latent virus 2 RNA 1, complete |
| | | sequence. |
| 790) | 18mers_human_m_virus_2MM_1079_p.txt | Spring beauty latent virus RNA 1, |
| | | complete sequence. |
| 791) | 18mers_human_m_virus_2MM_1102_p.txt | Aconitum latent virus, complete genome. |
| 792) | 18mers_human_m_virus_2MM_1216_p.txt | Pseudomonas phage PP7, complete |
| | | genome. |
| 793) | 18mers_human_m_virus_2MM_1250_p.txt | Porcine reproductive and respiratory |
| | | syndrome virus, complete genome. |
| 794) | 18mers_human_m_virus_2MM_1325_p.txt | Cocksfoot streak virus, complete genome. |
| 795) | 18mers_human_m_virus_2MM_1401_p.txt | Chikungunya virus, complete genome. |
| 796) | 18mers_human_m_virus_2MM_1483_p.txt | Staphylococcus aureus phage phi 11 |
| | | provirus, complete genome. |
| 797) | 18mers_human_m_virus_2MM_1492_p.txt | Streptococcus pyogenes phage 315.6 |
| | | provirus, complete genome. |
| 798) | 18mers_human_m_virus_2MM_236_p.txt | Streptococcus thermophilus bacteriophage |
| | | Sfi19, complete genome. |
| 799) | 18mers_human_m_virus_2MM_875_p.txt | Adeno-associated virus 4, complete |
| | | genome. |
| 800) | 18mers_human_m_virus_2MM_1049_p.txt | Brome mosaic virus RNA 1, complete |
| | | sequence. |
| 801) | 18mers_human_m_virus_2MM_1488_p.txt | Streptococcus pyogenes phage 315.2 |
| | | provirus, complete genome. |
| 802) | 18mers_human_m_virus_2MM_1019_p.txt | Garlic virus E, complete genome. |
| 803) | 18mers_human_m_virus_2MM_1196_p.txt | Sorghum chlorotic spot virus RNA 1, |
| | | complete sequence. |
| 804) | 18mers_human_m_virus_2MM_1210_p.txt | Enterobacteria phage fr, complete genome. |
| 805) | 18mers_human_m_virus_2MM_1283_p.txt | Foot-and-mouth disease virus SAT 2, |
| | | complete genome. |
| 806) | 18mers_human_m_virus_2MM_235_p.txt | Streptococcus thermophilus bacteriophage |
| | | Sfi11, complete genome. |
| 807) | 18mers_human_m_virus_2MM_812_p.txt | Propionibacterium phage phiB5, complete |
| | | genome. |
| 808) | 18mers_human_m_virus_2MM_1163_p.txt | Hepatitis G virus, complete genome. |
| 809) | 18mers_human_m_virus_2MM_1245_p.txt | Equine arteritis virus, complete genome. |
| 810) | 18mers_human_m_virus_2MM_1252_p.txt | Simian hemorrhagic fever virus, complete |
| | | genome. |
| 811) | 18mers_human_m_virus_2MM_1342_p.txt | Ryegrass mosaic virus, complete genome. |
| 812) | 18mers_human_m_virus_2MM_1402_p.txt | Eastern equine encephalitis virus, complete |
| | | genome. |
| 813) | 18mers_human_m_virus_2MM_1486_p.txt | Staphylococcus phage phiN315 provirus, |
| | | complete genome. |
| 814) | 18mers_human_m_virus_2MM_1405_p.txt | O'nyong-nyong virus, complete genome. |
| 815) | 18mers_human_m_virus_2MM_1410_p.txt | Sindbis virus, complete genome. |
| 816) | 18mers_human_m_virus_2MM_161_p.txt | Bacteriophage A118 virion, complete |
| | | genome. |
| 817) | 18mers_human_m_virus_2MM_1406_p.txt | Ross River virus, complete genome. |
| 818) | 18mers_human_m_virus_2MM_233_p.txt | Streptococcus thermophilus bacteriophage |
| | | 7201, complete genome. |
| 819) | 18mers_human_m_virus_2MM_278_p.txt | Sulfolobus islandicus filamentous virus, |
| | | genome. |
| 820) | 18mers_human_m_virus_2MM_813_p.txt | Pseudomonas phage Pf1, complete |
| | | genome. |
| 821) | 18mers_human_m_virus_2MM_1491_p.txt | Streptococcus pyogenes phage 315.5 |
| | | provirus, complete genome. |
| 822) | 18mers_human_m_virus_2MM_503_p.txt | Kadipiro virus segment 1, complete |
| | | sequence. |
| 823) | 18mers_human_m_virus_2MM_607_p.txt | Trichoplusia ni cytoplasmic polyhedrosis |
| | | virus 15 segment 1, complete |
| 824) | 18mers_human_m_virus_2MM_1241_p.txt | Saccharomyces cerevisiae narnavirus 20S |
| | | RNA &lt; W&gt;, complete genome. |
| 825) | 18mers_human_m_virus_2MM_1399_p.txt | Aura virus, complete genome. |
| 826) | 18mers_human_m_virus_2MM_1040_p.txt | American plum line pattern virus RNA1, |
| | | complete sequence. |
| 827) | 18mers_human_m_virus_2MM_1153_p.txt | Rhopalosiphum padi virus, complete |
| | | genome. |
| 828) | 18mers_human_m_virus_2MM_1381_p.txt | Kyuri green mottle mosaic virus, complete |
| | | genome. |
| 829) | 18mers_human_m_virus_2MM_1408_p.txt | Salmon pancreas disease virus, complete |
| | | genome. |
| 830) | 18mers_human_m_virus_2MM_240_p.txt | Temperate phage PhiN1H1.1, complete |
| | | genome. |
| 831) | 18mers_human_m_virus_2MM_245_p.txt | Alteromonas phage PM2, complete |
| | | genome. |
| 832) | 18mers_human_m_virus_2MM_411_p.txt | Yaba-like disease virus, complete genome. |
| 833) | 18mers_human_m_virus_2MM_806_p.txt | Bacteriophage VfO4K68, complete |
| | | genome. |
| 834) | 18mers_human_m_virus_2MM_1490_p.txt | Streptococcus pyogenes phage 315.4 |
| | | provirus, complete genome. |
| 835) | 18mers_human_m_virus_2MM_137_p.txt | Porcine adenovirus A, complete genome. |
| 836) | 18mers_human_m_virus_2MM_805_p.txt | Bacteriophage VfO3K6, complete genome. |
| 837) | 18mers_human_m_virus_2MM_1215_p.txt | Enterobacterio phage MS2, complete |
| | | genome. |
| 838) | 18mers_human_m_virus_2MM_1262_p.txt | Nodamura virus RNA1, complete |
| | | sequence. |
| 839) | 18mers_human_m_virus_2MM_177_p.txt | Bacteriophage P4, complete genome. |
| 840) | 18mers_human_m_virus_2MM_1409_p.txt | Semliki forest virus, complete genome. |
| 841) | 18mers_human_m_virus_2MM_134_p.txt | Murine adenovirus A, complete genome. |
| 842) | 18mers_human_m_virus_2MM_138_p.txt | Porcine adenovirus C, complete genome. |
| 843) | 18mers_human_m_virus_2MM_621_p.txt | Helminthosporium victoriae virus 190S, |
| | | complete genome. |
| 844) | 18mers_human_m_virus_2MM_1375_p.txt | Helicoverpa armigera stunt virus RNA 1, |
| | | complete sequence. |
| 845) | 18mers_human_m_virus_2MM_136_p.txt | Ovine adenovirus A, complete genome. |
| 846) | 18mers_human_m_virus_2MM_1407_p.txt | Rubella virus, complete genome. |
| 847) | 18mers_human_m_virus_2MM_479_p.txt | Colorado tick fever virus segment 1, |
| | | complete sequence. |
| 848) | 18mers_human_m_virus_2MM_491_p.txt | Eyach virus segment 1, complete sequence. |
| 849) | 18mers_human_m_virus_2MM_1411_p.txt | Sleeping disease virus, complete genome. |
| 850) | 18mers_human_m_virus_2MM_124_p.txt | Duck adenovirus 1, complete genome. |
| 851) | 18mers_human_m_virus_2MM_437_p.txt | Cryphonectria hypovirus 1, complete |
| | | genome. |
| 852) | 18mers_human_m_virus_2MM_1214_p.txt | Enterobacteria phage NL95. |
| 853) | 18mers_human_m_virus_2MM_585_p.txt | Rice dwarf virus segment 1, complete |
| | | sequence. |
| 854) | 18mers_human_m_virus_2MM_627_p.txt | Sphaeropsis sapinea RNA virus 1, |
| | | complete genome. |
| 855) | 18mers_human_m_virus_2MM_408_p.txt | Swinepox virus, complete genome. |
| 856) | 18mers_human_m_virus_2MM_535_p.txt | Mammalian orthoreovirus 1 segment L1, |
| | | complete sequence. |
| 857) | 18mers_human_m_virus_2MM_121_p.txt | Bovine adenovirus B, complete genome. |
| 858) | 18mers_human_m_virus_2MM_129_p.txt | Human adenovirus B, complete genome. |
| 859) | 18mers_human_m_virus_2MM_204_p.txt | Haemophilus phage HP2, complete |
| | | genome. |
| 860) | 18mers_human_m_virus_2MM_628_p.txt | Sphaeropsis sapinea RNA virus 2, |
| | | complete genome. |
| 861) | 18mers_human_m_virus_2MM_1209_p.txt | Bacteriophage SP, complete genome. |
| 862) | 18mers_human_m_virus_2MM_822_p.txt | Xanthomonas phage Cf1c, complete |
| | | genome. |
| 863) | 18mers_human_m_virus_2MM_128_p.txt | Human adenovirus A, complete genome. |
| 864) | 18mers_human_m_virus_2MM_132_p.txt | Human adenovirus E, complete genome. |
| 865) | 18mers_human_m_virus_2MM_241_p.txt | Vibrio harveyi bacteriophage VHML, |
| | | complete genome. |
| 866) | 18mers_human_m_virus_2MM_414_p.txt | Enterobacteria phage PRD1, complete |
| | | genome. |
| 867) | 18mers_human_m_virus_2MM_436_p.txt | Cryphonectria hypovirus, complete |
| | | genome. |
| 868) | 18mers_human_m_virus_2MM_203_p.txt | Haemophilus phage HP1, complete |
| | | genome. |
| 869) | 18mers_human_m_virus_2MM_555_p.txt | Mammalian orthoreovirus 3 segment L1, |
| | | complete sequence. |
| 870) | 18mers_human_m_virus_2MM_1377_p.txt | Nudaurelia capensis beta virus, complete |
| | | genome. |
| 871) | 18mers_human_m_virus_2MM_131_p.txt | Human adenovirus D, complete genome. |
| 872) | 18mers_human_m_virus_2MM_180_p.txt | Bacteriophage phi-105, complete genome. |
| 873) | 18mers_human_m_virus_2MM_265_p.txt | Human herpesvirus 7, complete genome. |
| 874) | 18mers_human_m_virus_2MM_191_p.txt | Bacteriophage T3, complete genome. |
| 875) | 18mers_human_m_virus_2MM_244_p.txt | Yersinia pestis phage phiA1122, complete |
| | | genome. |
| 876) | 18mers_human_m_virus_2MM_515_p.txt | Lymantria dispar cypovirus 1 segment 1, |
| | | complete sequence. |
| 877) | 18mers_human_m_virus_2MM_597_p.txt | Rice ragged stunt virus segment 1, |
| | | complete sequence. |
| 878) | 18mers_human_m_virus_2MM_219_p.txt | Pseudomonas aeruginosa phage PaP3, |
| | | complete genome. |
| 879) | 18mers_human_m_virus_2MM_242_p.txt | Vibriophage VpV262, complete genome. |
| 880) | 18mers_human_m_virus_2MM_276_p.txt | Lymphocystis disease virus 1, complete |
| | | genome. |
| 881) | 18mers_human_m_virus_2MM_206_p.txt | Lactobacillus casei bacteriophage A2 |
| | | virion, complete genome. |
| 882) | 18mers_human_m_virus_2MM_525_p.txt | Lymantria dispar cypovirus 14 segment I, |
| | | complete sequence. |
| 883) | 18mers_human_m_virus_2MM_202_p.txt | Enterobacteria phage T7. |
| 884) | 18mers_human_m_virus_2MM_130_p.txt | Human adenovirus C, complete genome. |
| 885) | 18mers_human_m_virus_2MM_185_p.txt | Bacteriophage phigle, complete genome. |
| 886) | 18mers_human_m_virus_2MM_469_p.txt | Bombyx mori cypovirus 1 segment 1, |
| | | complete sequence. |
| 887) | 18mers_human_m_virus_2MM_1111_p.txt | Grapevine leafroll-associated virus 3, |
| | | complete genome. |
| 888) | 18mers_human_m_virus_2MM_186_p.txt | Bacteriophage phiYeO3-12, complete |
| | | genome. |
| 889) | 18mers_human_m_virus_2MM_255_p.txt | Equine herpesvirus 2, complete genome. |
| 890) | 18mers_human_m_virus_2MM_133_p.txt | Human adenovirus F, complete genome. |
| 891) | 18mers_human_m_virus_2MM_172_p.txt | Bacteriophage K139, complete genome. |
| 892) | 18mers_human_m_virus_2MM_190_p.txt | Bacteriophage SPP1, complete genome. |
| 893) | 18mers_human_m_virus_2MM_170_p.txt | Bacteriophage HK620, complete genome. |
| 894) | 18mers_human_m_virus_2MM_1107_p.txt | Beet yellows virus, complete genome. |
| 895) | 18mers_human_m_virus_2MM_545_p.txt | Mammalian orthoreovirus 2 segment L1, |
| | | complete sequence. |
| 896) | 18mers_human_m_virus_2MM_1112_p.txt | Grapevine rootstock stem lesion associated |
| | | virus, complete genome. |
| 897) | 18mers_human_m_virus_2MM_261_p.txt | Human herpesvirus 4, complete genome. |
| 898) | 18mers_human_m_virus_2MM_139_p.txt | Tupaia adenovirus 1, complete genome. |
| 899) | 18mers_human_m_virus_2MM_224_p.txt | Salmonella typhimurium bacteriophage |
| | | ST64T virion, complete genome. |
| 900) | 18mers_human_m_virus_2MM_201_p.txt | Enterobacteria phage T4, complete |
| | | genome. |
| 901) | 18mers_human_m_virus_2MM_277_p.txt | Rana tigrina ranavirus, complete genome. |
| 902) | 18mers_human_m_virus_2MM_1108_p.txt | Citrus tristeza virus, complete genome. |
| 903) | 18mers_human_m_virus_2MM_171_p.txt | Bacteriophage HK97, complete genome. |
| 904) | 18mers_human_m_virus_2MM_221_p.txt | Pseudomonas phage gh-1, complete |
| | | genome. |
| 905) | 18mers_human_m_virus_2MM_226_p.txt | Shigella flexneri bacteriophage V, |
| | | complete genome. |
| 906) | 18mers_human_m_virus_2MM_198_p.txt | Enterobacteria phage H K022 virion, |
| | | complete genome. |
| 907) | 18mers_human_m_virus_2MM_279_p.txt | Shrimp white spot syndrome virus, |
| | | complete genome. |
| 908) | 18mers_human_m_virus_2MM_173_p.txt | Bacteriophage L-413C, complete genome. |
| 909) | 18mers_human_m_virus_2MM_251_p.txt | Callitrichine herpesvirus 3, complete |
| | | genome. |
| 910) | 18mers_human_m_virus_2MM_157_p.txt | Acyrthosiphon pisum bacteriophage |
| | | APSE-1, complete genome. |
| 911) | 18mers_human_m_virus_2MM_427_p.txt | Bacteriophage phi-8 segment L, complete |
| | | sequence. |
| 912) | 18mers_human_m_virus_2MM_199_p.txt | Enterobacteria phage Mu, complete |
| | | genome. |
| 913) | 18mers_human_m_virus_2MM_410_p.txt | Variola virus, complete genome. |
| 914) | 18mers_human_m_virus_2MM_200_p.txt | Enterobacteria phage P2, complete |
| | | genome. |
| 915) | 18mers_human_m_virus_2MM_433_p.txt | Pseudomonas phage phi-6 segment L, |
| | | complete sequence. |
| 916) | 18mers_human_m_virus_2MM_176_p.txt | Bacteriophage P27, complete genome. |
| 917) | 18mers_human_m_virus_2MM_197_p.txt | Enterobacteria phage epsilon 15, complete |
| | | genome. |
| 918) | 18mers_human_m_virus_2MM_424_p.txt | Bacteriophage phi-12 segment L, complete |
| | | sequence. |
| 919) | 18mers_human_m_virus_2MM_404_p.txt | Monkeypox virus, complete genome. |
| 920) | 18mers_human_m_virus_2MM_174_p.txt | Bacteriophage lambda, complete genome. |
| 921) | 18mers_human_m_virus_2MM_430_p.txt | Pseudomonas bacteriophage phi-I3 |
| | | segment L, complete sequence. |
| 922) | 18mers_human_m_virus_2MM_222_p.txt | Pseudomonas phage phi KZ, complete |
| | | genome. |
| 923) | 18mers_human_m_virus_2MM_160_p.txt | Bacteriophage 933W provirus, complete |
| | | genome. |
| 924) | 18mers_human_m_virus_2MM_239_p.txt | Stx2 converting bacteriophage I virion, |
| | | complete genome. |
| 925) | 18mers_human_m_virus_2MM_194_p.txt | Bacteriophage VT2-Sa provirus, complete |
| | | genome. |
| 926) | 18mers_human_m_virus_2MM_196_p.txt | Enterobacteria phage 186, complete |
| | | genome. |
| 927) | 18mers_human_m_virus_2MM_225_p.txt | Salmonella typhimurium phage ST64B, |
| | | complete genome. |
| 928) | 18mers_human_m_virus_2MM_397_p.txt | Camelpox virus, complete genome. |
| 929) | 18mers_human_m_virus_2MM_409_p.txt | Vaccinia virus, complete genome. |
| 930) | 18mers_human_m_virus_2MM_125_p.txt | Fowl adenovirus A, complete genome. |
| 931) | 18mers_human_m_virus_2MM_399_p.txt | Ectromelia virus, complete genome. |
| 932) | 18mers_human_m_virus_2MM_252_p.txt | Cercopithecine herpesvirus 7, complete |
| | | genome. |
| 933) | 18mers_human_m_virus_2MM_398_p.txt | Cowpox virus, complete genome. |
| 934) | 18mers_human_m_virus_2MM_178_p.txt | Bacteriophage phi CTX, complete genome. |
| 935) | 18mers_human_m_virus_2MM_175_p.txt | Bacteriophage N15 virion, complete |
| | | genome. |
| 936) | 18mers_human_m_virus_2MM_400_p.txt | Fowlpox virus, complete genome. |
| 937) | 18mers_human_m_virus_2MM_1471_p.txt | Bacteriophage Mx8, complete genome. |
| 938) | 18mers_human_m_virus_2MM_274_p.txt | Infectious spleen and kidney necrosis virus, |
| | | complete genome. |
| 939) | 18mers_human_m_virus_2MM_257_p.txt | Gallid herpesvirus 2, complete genome. |
| 940) | 18mers_human_m_virus_2MM_266_p.txt | Human herpesvirus 8, genome. |
| 941) | 18mers_human_m_virus_2MM_141_p.txt | Adoxophyes honmai |
| | | nucleopolyhedrovirus, complete genome. |
| 942) | 18mers_human_m_virus_2MM_220_p.txt | Pseudomonas phage D3, complete genome. |
| 943) | 18mers_human_m_virus_2MM_263_p.txt | Human herpesvirus 6, complete genome. |
| 944) | 18mers_human_m_virus_2MM_264_p.txt | Human herpesvirus 6B, complete genome. |
| 945) | 18mers_human_m_virus_2MM_217_p.txt | Mycobacterium phage L5. |
| 946) | 18mers_human_m_virus_2MM_1474_p.txt | Mycobacteriophage Bxz2 virion, complete |
| | | genome. |
| 947) | 18mers_human_m_virus_2MM_1481_p.txt | Mycobacteriophage Rosebush virion, |
| | | complete genome. |
| 948) | 18mers_human_m_virus_2MM_1478_p.txt | Mycobacteriophage CJWI virion, complete |
| | | genome. |
| 949) | 18mers_human_m_virus_2MM_256_p.txt | Equine herpesvirus 4, complete genome. |
| 950) | 18mers_human_m_virus_2MM_126_p.txt | Fowl adenovirus D, complete genome. |
| 951) | 18mers_human_m_virus_2MM_215_p.txt | Mycobacteriophage D29, complete |
| | | genome. |
| 952) | 18mers_human_m_virus_2MM_260_p.txt | Human herpesvirus 3, complete genome. |
| 953) | 18mers_human_m_virus_2MM_153_p.txt | Plutella xylostella granulovirus, complete |
| | | genome. |
| 954) | 18mers_human_m_virus_2MM_1475_p.txt | Mycobacteriophage Che8 virion, complete |
| | | genome. |
| 955) | 18mers_human_m_virus_2MM_1477_p.txt | Mycobacteriophage Che9d virion, |
| | | complete genome. |
| 956) | 18mers_human_m_virus_2MM_1479_p.txt | Mycobacteriophage Corndog virion, |
| | | complete genome. |
| 957) | 18mers_human_m_virus_2MM_1482_p.txt | Sinorhizobium meliloti phage PBC5, |
| | | complete genome. |
| 958) | 18mers_human_m_virus_2MM_254_p.txt | Equine herpesvirus 1, complete genome. |
| 959) | 18mers_human_m_virus_2MM_154_p.txt | Rachiplusia ou multiple |
| | | nucleopolyhedrovirus, complete genome. |
| 960) | 18mers_human_m_virus_2MM_181_p.txt | Bacteriophage phi-BTI virion, complete |
| | | genome. |
| 961) | 18mers_human_m_virus_2MM_214_p.txt | Mycobacteriophage Bxb1, complete |
| | | genome. |
| 962) | 18mers_human_m_virus_2MM_249_p.txt | Bovine herpesvirus 1, complete genome. |
| 963) | 18mers_human_m_virus_2MM_1476_p.txt | Mycobacteriophage Che9c virion, |
| | | complete genome. |
| 964) | 18mers_human_m_virus_2MM_216_p.txt | Mycobacteriophage TM4, complete |
| | | genome. |
| 965) | 18mers_human_m_virus_2MM_268_p.txt | Meleagrid herpesvirus 1, complete |
| | | genome. |
| 966) | 18mers_human_m_virus_2MM_182_p.txt | Bacteriophage phi-C31, complete genome. |
| 967) | 18mers_human_m_virus_2MM_1472_p.txt | Mycobacteriophage Barnyard virion, |
| | | complete genome. |
| 968) | 18mers_human_m_virus_2MM_143_p.txt | Bombyx mori nuclear polyhedrosis virus, |
| | | complete genome. |
| 969) | 18mers_human_m_virus_2MM_142_p.txt | Autographa califomica |
| | | nucleopolyhedrovirus, complete genome. |
| 970) | 18mers_human_m_virus_2MM_145_p.txt | Culex nigripalpus baculovirus, complete |
| | | genome. |
| 971) | 18mers_human_m_virus_2MM_386_p.txt | Paramecium bursaria Chlorella virus 1, |
| | | complete genome. |
| 972) | 18mers_human_m_virus_2MM_259_p.txt | Human herpesvirus 2, complete genome. |
| 973) | 18mers_human_m_virus_2MM_156_p.txt | Xestia c-nigrum granulovirus, complete |
| | | genome. |
| 974) | 18mers_human_m_virus_2MM_146_p.txt | Epiphyas postvittana |
| | | nucleopolyhedrovirus, complete genome. |
| 975) | 18mers_human_m_virus_2MM_267_p.txt | Ictalurid herpesvirus 1, complete genome. |
| 976) | 18mers_human_m_virus_2MM_406_p.txt | Rabbit fibroma virus, complete genome. |
| 977) | 18mers_human_m_virus_2MM_148_p.txt | Helicoverpa zea single nucleocapsid |
| | | nucleopolyhedrovirus, complete genome. |
| 978) | 18mers_human_m_virus_2MM_149_p.txt | Heliocoverpa armigera |
| | | nucleopolyhedrovirus G4, complete genome. |
| 979) | 18mers_human_m_virus_2MM_147_p.txt | Helicoverpa armigera nuclear polyhedrosis |
| | | virus, complete genome. |
| 980) | 18mers_human_m_virus_2MM_151_p.txt | Mamestra configurata |
| | | nucleopolyhedrovirus B, complete genome. |
| 981) | 18mers_human_m_virus_2MM_1480_p.txt | Mycobacteriophage Omega virion, |
| | | complete genome. |
| 982) | 18mers_human_m_virus_2MM_150_p.txt | Mamestra configurata |
| | | nucleopolyhedrovirus A, complete genome. |
| 983) | 18mers_human_m_virus_2MM_1473_p.txt | Mycobacteriophage Bxzl virion, complete |
| | | genome. |
| 984) | 18mers_human_m_virus_2MM_184_p.txt | Bacteriophage phiE125, complete genome. |
| 985) | 18mers_human_m_virus_2MM_195_p.txt | Burkholderia cepacia phage Bcep781, |
| | | complete genome. |
| 986) | 18mers_human_m_virus_2MM_152_p.txt | Orgyia pseudotsugata multicapsid |
| | | nucleopolyhedrovirus, complete genome. |
| 987) | 18mers_human_m_virus_2MM_144_p.txt | Choristoneura fumiferana MNPV, |
| | | complete genome. |
| 988) | 18mers_human_m_virus_2MM_253_p.txt | Chimpanzee cytomegalovirus, complete |
| | | genome. |
| 989) | 18mers_human_m_virus_2MM_243_p.txt | Virus PhiCh1, complete genome. |
| 990) | 18mers_human_m_virus_2MM_258_p.txt | Gallid herpesvirus 3, complete genome. |
| 991) | 18mers_human_m_virus_2MM_155_p.txt | Spodoptera exigua nucleopolyhedrovirus, |
| | | complete genome. |
| 992) | 18mers_human_m_virus_2MM_262_p.txt | Human herpesvirus 5, complete genome. |
| 993) | 18mers_human_m_virus_2MM_271_p.txt | Rat cytomegalovirus, complete genome. |
| 994) | 18mers_human_m_virus_2MM_273_p.txt | Tupaia herpesvirus, complete genome. |
| 995) | 18mers_human_m_virus_2MM_385_p.txt | Ectocarpus siliculosus virus, complete |
| | | genome. |
| 996) | 18mers_human_m_virus_2MM_405_p.txt | Myxoma virus, complete genome. |
| 997) | 18mers_human_m_virus_2MM_269_p.txt | Mouse cytomegalovirus 1, complete |
| | | genome. |

2) SEQ ID NO 5: Virus 18 mers 1 MM - It contains 1095 files, containing
sequences of length 18, which are present in the viral genome, but are not present
in the human genome. All sequences produced from these sequences with 1
mismatch are also not present in the human genome. The list below contains the
file name and the corresponding Virus name.

| | File Name | species taxonomy name |
|---|---|---|
| 1) | 18mers_human_m_virus_1MM_1_p.txt | Hepatitis D virus, complete genome. |
| 2) | 18mers_human_m_virus_1MM_2_p.txt | Banana streak virus, complete genome. |
| 3) | 18mers_human_m_virus_1MM_3_p.txt | Blueberry red ringspot virus, complete genome. |
| 4) | 18mers_human_m_virus_1MM_4_p.txt | Cacao swollen shoot virus, complete genome. |
| 5) | 18mers_human_m_virus_1MM_5_p.txt | Carnation etched ring virus, complete genome. |
| 6) | 18mers_human_m_virus_1MM_6_p.txt | Cassava vein mosaic virus, complete genome. |
| 7) | 18mers_human_m_virus_1MM_7_p.txt | Cauliflower mosaic virus, complete genome. |
| 8) | 18mers_human_m_virus_1MM_8_p.txt | Cestrum yellow leaf curling virus, complete genome. |
| 9) | 18mers_human_m_virus_1MM_9_p.txt | Citrus yellow mosaic virus, complete genome. |
| 10) | 18mers_human_m_virus_1MM_10_p.txt | Commelina yellow mottle virus, complete genome. |
| 11) | 18mers_human_m_virus_1MM_11_p.txt | Figwort mosaic virus, complete genome. |
| 12) | 18mers_human_m_virus_1MM_12_p.txt | Kalanchoe top-spotting virus, complete genome. |
| 13) | 18mers_human_m_virus_1MM_13_p.txt | Mirabilis mosaic virus, complete genome. |
| 14) | 18mers_human_m_virus_1MM_14_p.txt | Peanut chlorotic streak virus, complete genome. |
| 15) | 18mers_human_m_virus_1MM_15_p.txt | Petunia vein clearing virus, complete genome. |
| 16) | 18mers_human_m_virus_1MM_16_p.txt | Rice tungro bacilliform virus, complete genome. |
| 17) | 18mers_human_m_virus_1MM_17_p.txt | Soybean chlorotic mottle virus, complete genome. |
| 18) | 18mers_human_m_virus_1MM_18_p.txt | Strawberry vein banding virus, complete genome. |
| 19) | 18mers_human_m_virus_1MM_19_p.txt | Sugarcane bacilliform virus, complete genome. |
| 20) | 18mers_human_m_virus_1MM_20_p.txt | Taro bacilliform virus, complete genome. |
| 21) | 18mers_human_m_virus_1MM_21_p.txt | Tobacco vein-clearing virus, complete genome. |
| 22) | 18mers_human_m_virus_1MM_22_p.txt | Arctic ground squirrel hepatitis B virus, complete |
| | | genome. |
| 23) | 18mers_human_m_virus_1MM_23_p.txt | Duck hepatitis B virus, complete genome. |
| 24) | 18mers_human_m_virus_1MM_24_p.txt | Ground squirrel hepatitis virus, complete genome. |
| 25) | 18mers_human_m_virus_1MM_25_p.txt | Hepatitis B virus, complete genome. |
| 26) | 18mers_human_m_virus_1MM_26_p.txt | Heron hepatitis B virus, complete genome. |
| 27) | 18mers_human_m_virus_1MM_27_p.txt | Orangutan hepadnavirus, complete genome. |
| 28) | 18mers_human_m_virus_1MM_28_p.txt | Woodchuck hepatitis B virus, complete genome. |
| 29) | 18mers_human_m_virus_1MM_29_p.txt | Woolly monkey hepatitis B Virus, complete genome. |
| 30) | 18mers_human_m_virus_1MM_30_p.txt | Abelson murine leukemia virus, complete genome. |
| 31) | 18mers_human_m_virus_1MM_31_p.txt | Avian carcinoma virus, complete genome. |
| 32) | 18mers_human_m_virus_1MM_32_p.txt | Avian leukosis virus, complete genome. |
| 33) | 18mers_human_m_virus_1MM_33_p.txt | Avian myelocytomatosis virus, complete genome. |
| 34) | 18mers_human_m_virus_1MM_34_p.txt | Avian sarcoma virus, complete genome. |
| 35) | 18mers_human_m_virus_1MM_35_p.txt | Bovine foamy virus, complete genome. |
| 36) | 18mers_human_m_virus_1MM_36_p.txt | Bovine immunodeficiency virus, complete genome. |
| 37) | 18mers_human_m_virus_1MM_37_p.txt | Bovine leukemia virus, complete genome. |
| 38) | 18mers_human_m_virus_1MM_38_p.txt | Caprine arthritis-encephalitis virus, complete genome. |
| 39) | 18mers_human_m_virus_1MM_39_p.txt | Equine foamy virus, complete genome. |
| 40) | 18mers_human_m_virus_1MM_40_p.txt | Equine infectious anemia virus, complete genome. |
| 41) | 18mers_human_m_virus_1MM_41_p.txt | Feline foamy virus, complete genome. |
| 42) | 18mers_human_m_virus_1MM_42_p.txt | Feline immunodeficiency virus, complete genome. |
| 43) | 18mers_human_m_virus_1MM_43_p.txt | Feline leukemia virus, complete genome. |
| 44) | 18mers_human_m_virus_1MM_44_p.txt | Friend murine leukemia virus, complete genome. |
| 45) | 18mers_human_m_virus_1MM_45_p.txt | Fujinami sarcoma virus, complete genome. |
| 46) | 18mers_human_m_virus_1MM_46_p.txt | Gibbon ape leukemia virus, complete genome. |
| 47) | 18mers_human_m_virus_1MM_47_p.txt | Human foamy virus, complete genome. |
| 48) | 18mers_human_m_virus_1MM_48_p.txt | Human immunodeficiency virus 1, complete genome. |
| 49) | 18mers_human_m_virus_1MM_49_p.txt | Human immunodeficiency virus 2, complete genome. |
| 50) | 18mers_human_m_virus_1MM_50_p.txt | Human spumaretrovirus, complete genome. |
| 51) | 18mers_human_m_virus_1MM_51_p.txt | Human T-lymphotropic virus 1, complete genome. |
| 52) | 18mers_human_m_virus_1MM_52_p.txt | Human T-lymphotropic virus 2, complete genome. |
| 53) | 18mers_human_m_virus_1MM_53_p.txt | Jembrana disease virus, complete genome. |
| 54) | 18mers_human_m_virus_1MM_54_p.txt | Mason-Pfizer monkey virus, complete genome. |
| 55) | 18mers_human_m_virus_1MM_55_p.txt | Moloney murine sarcoma virus, complete genome. |
| 56) | 18mers_human_m_virus_1MM_56_p.txt | Mouse mammary tumor virus, complete genome. |
| 57) | 18mers_human_m_virus_1MM_57_p.txt | Murine leukemia virus, complete genome. |
| 58) | 18mers_human_m_virus_1MM_58_p.txt | Murine osteosarcoma virus, complete genome. |
| 59) | 18mers_human_m_virus_1MM_59_p.txt | Murine sarcoma virus, complete genome. |
| 60) | 18mers_human_m_virus_1MM_60_p.txt | Murine type C retrovirus, complete genome. |
| 61) | 18mers_human_m_virus_1MM_61_p.txt | Ovine lentivirus, complete genome. |
| 62) | 18mers_human_m_virus_1MM_62_p.txt | Ovine pulmonary adenocarcinoma virus, complete |
| | | genome. |
| 63) | 18mers_human_m_virus_1MM_63_p.txt | Porcine endogenous retrovirus, complete genome. |
| 64) | 18mers_human_m_virus_1MM_64_p.txt | Rauscher murine leukemia virus, complete genome. |
| 65) | 18mers_human_m_virus_1MM_65_p.txt | Rous sarcoma virus, complete genome. |
| 66) | 18mers_human_m_virus_1MM_66_p.txt | Simian foamy virus, complete genome. |
| 67) | 18mers_human_m_virus_1MM_67_p.txt | Simian immunodeficiency virus, complete genome. |
| 68) | 18mers_human_m_virus_1MM_68_p.txt | Simian immunodeficiency virus 2, complete genome. |
| 69) | 18mers_human_m_virus_1MM_69_p.txt | Simian T-lymphotropic virus 1, complete genome. |
| 70) | 18mers_human_m_virus_1MM_70_p.txt | Simian T-lymphotropic virus 2, complete genome. |
| 71) | 18mers_human_m_virus_1MM_71_p.txt | Simian-Human immunodeficiency virus, complete |
| | | genome. |
| 72) | 18mers_human_m_virus_1MM_72_p.txt | Snakehead retrovirus, complete genome. |
| 73) | 18mers_human_m_virus_1MM_73_p.txt | Spleen focus-forming virus, complete genome. |
| 74) | 18mers_human_m_virus_1MM_74_p.txt | Visna virus, complete genome. |
| 75) | 18mers_human_m_virus_1MM_75_p.txt | Walleye dermal sarcoma virus, complete genome. |
| 76) | 18mers_human_m_virus_1MM_76_p.txt | Woolly monkey sarcoma virus, complete genome. |
| 77) | 18mers_human_m_virus_1MM_77_p.txt | Y73 sarcoma virus, complete genome. |
| 78) | 18mers_human_m_virus_1MM_78_p.txt | Ageratum yellow vein virus-associated DNA beta. |
| | | complete genome. |
| 79) | 18mers_human_m_virus_1MM_79_p.txt | Arabis mosaic virus satellite RNA, complete genome. |
| 80) | 18mers_human_m_virus_1MM_80_p.txt | Arabis mosaic virus small satellite RNA, complete |
| | | genome. |
| 81) | 18mers_human_m_virus_1MM_81_p.txt | Bamboo mosaic virus satellite RNA, complete genome. |
| 82) | 18mers_human_m_virus_1MM_82_p.txt | Bhendi yellow vein mosaic virus-associated DNA beta, |
| | | complete genome. |
| 83) | 18mers_human_m_virus_1MM_83_p.txt | Cereal yellow dwarf virus-RPV satellite RNA, complete |
| | | genome. |
| 84) | 18mers_human_m_virus_1MM_84_p.txt | Chicory yellow mottle virus large satellite RNA. complete |
| | | genome. |
| 85) | 18mers_human_m_virus_1MM_85_p.txt | Chicory yellow mottle virus satellite RNA, complete |
| | | genome. |
| 86) | 18mers_human_m_virus_1MM_86_p.txt | Cotton leaf curl virus-associated DNA beta, complete |
| | | genome. |
| 87) | 18mers_human_m_virus_1MM_87_p.txt | Cucumber mosaic virus satellite RNA, complete genome. |
| 88) | 18mers_human_m_virus_1MM_88_p.txt | Cymbidium ringspot virus satellite RNA, complete |
| | | genome. |
| 89) | 18mers_human_m_virus_1MM_89_p.txt | Eupatorium yellow vein virus-associated DNA beta, |
| | | complete genome. |
| 90) | 18mers_human_m_virus_1MM_90_p.txt | Grapevine fanleaf virus satellite RNA, complete genome. |
| 91) | 18mers_human_m_virus_1MM_91_p.txt | Hollyhock leaf crumple virus satellite DNA, complete |
| | | genome. |
| 92) | 18mers_human_m_virus_1MM_92_p.txt | Lucerne transient streak virus satellite RNA, complete |
| | | genome. |
| 93) | 18mers_human_m_virus_1MM_93_p.txt | Malvastrum yellow vein virus-associated DNA beta, |
| | | complete genome. |
| 94) | 18mers_human_m_virus_1MM_94_p.txt | OkLCV satDNA 10, complete genome. |
| 95) | 18mers_human_m_virus_1MM_95_p.txt | OkLCV satDNA 27, complete genome. |
| 96) | 18mers_human_m_virus_1MM_96_p.txt | OkLCV satDNA 3, complete genome. |
| 97) | 18mers_human_m_virus_1MM_97_p.txt | Panicum mosaic satellite virus, complete genome. |
| 98) | 18mers_human_m_virus_1MM_98_p.txt | Papaya leaf curl virus-associated DNA beta, complete |
| | | genome. |
| 99) | 18mers_human_m_virus_1MM_99_p.txt | Pea enation mosaic virus satellite RNA, complete |
| | | genome. |
| 100) | 18mers_human_m_virus_1MM_100_p.txt | Peanut stunt virus satellite RNA, complete genome. |
| 101) | 18mers_human_m_virus_1MM_101_p.txt | Rice yellow mottle virus satellite, complete genome. |
| 102) | 18mers_human_m_virus_1MM_102_p.txt | Satellite virus of maize white line mosaic virus, complete |
| | | genome. |
| 103) | 18mers_human_m_virus_1MM_103_p.txt | Satellite of Trichomonas vaginalis T1 virus, complete |
| | | genome. |
| 104) | 18mers_human_m_virus_1MM_104_p.txt | Satellite RNA of Blackcurrant reversion virus, complete |
| | | genome. |
| 105) | 18mers_human_m_virus_1MM_105_p.txt | Satellite RNA of Groundnut Rosette Virus, complete |
| | | genome. |
| 106) | 18mers_human_m_virus_1MM_106_p.txt | Satellite tobacco mosaic virus, complete genome. |
| 107) | 18mers_human_m_virus_1MM_107_p.txt | Solanum nodiflorum mottle virus satellite RNA, complete |
| | | genome. |
| 108) | 18mers_human_m_virus_1MM_108_p.txt | Strawberry latent ringspot virus satellite RNA, complete |
| | | genome. |
| 109) | 18mers_human_m_virus_1MM_109_p.txt | Subterranean clover mottle virus satellite RNA, complete |
| | | genome. |
| 110) | 18mers_human_m_virus_1MM_110_p.txt | Tobacco curly shoot virus-associated DNA beta, complete |
| | | genome. |
| 111) | 18mers_human_m_virus_1MM_111_p.txt | Tobacco necrosis satellite virus, complete genome. |
| 112) | 18mers_human_m_virus_1MM_112_p.txt | Tobacco ringspot virus satellite RNA, complete genome. |
| 113) | 18mers_human_m_virus_1MM_113_p.txt | Tomato black ring virus satellite RNA, complete genome. |
| 114) | 18mers_human_m_virus_1MM_114_p.txt | Tomato bushy stunt virus satellite RNA B1, complete |
| | | genome. |
| 115) | 18mers_human_m_virus_1MM_115_p.txt | Tomato bushy stunt virus satellite RNA B10, complete |
| | | genome. |
| 116) | 18mers_human_m_virus_1MM_116_p.txt | Tomato leaf curl virus satellite DNA, complete genome. |
| 117) | 18mers_human_m_virus_1MM_117_p.txt | Tomato leaf curl virus-associated DNA beta, complete |
| | | genome. |
| 118) | 18mers_human_m_virus_1MM_118_p.txt | Tomato yellow leaf curl China virus-associated DNA |
| | | beta, complete genome. |
| 119) | 18mers_human_m_virus_1MM_119_p.txt | Turnip crinkle virus satellite RNA, complete genome. |
| 120) | 18mers_human_m_virus_1MM_120_p.txt | Velvet tobacco mottle virus Satellite RNA, complete |
| | | genome. |
| 121) | 18mers_human_m_virus_1MM_121_p.txt | Bovine adenovirus B, complete genome. |
| 122) | 18mers_human_m_virus_1MM_122_p.txt | Bovine adenovirus D, complete genome. |
| 123) | 18mers_human_m_virus_1MM_123_p.txt | Canine adenovirus type 1, complete genome. |
| 124) | 18mers_human_m_virus_1MM_124_p.txt | Duck adenovirus 1, complete genome. |
| 125) | 18mers_human_m_virus_1MM_125_p.txt | Fowl adenovirus A, complete genome. |
| 126) | 18mers_human_m_virus_1MM_126_p.txt | Fowl adenovirus D, complete genome. |
| 127) | 18mers_human_m_virus_1MM_127_p.txt | Frog adenovirus 1, complete genome. |
| 128) | 18mers_human_m_virus_1MM_128_p.txt | Human adenovirus A, complete genome. |
| 129) | 18mers_human_m_virus_1MM_129_p.txt | Human adenovirus B, complete genome. |
| 130) | 18mers_human_m_virus_1MM_130_p.txt | Human adenovirus C, complete genome. |
| 131) | 18mers_human_m_virus_1MM_131_p.txt | Human adenovirus D, complete genome. |
| 132) | 18mers_human_m_virus_1MM_132_p.txt | Human adenovirus E, complete genome. |
| 133) | 18mers_human_m_virus_1MM_133_p.txt | Human adenovirus F, complete genome. |
| 134) | 18mers_human_m_virus_1MM_134_p.txt | Murine adenovirus A, complete genome. |
| 135) | 18mers_human_m_virus_1MM_135_p.txt | Ovine adenovirus 7, complete genome. |
| 136) | 18mers_human_m_virus_1MM_136_p.txt | Ovine adenovirus A, complete genome. |
| 137) | 18mers_human_m_virus_1MM_137_p.txt | Porcine adenovirus A, complete genome. |
| 138) | 18mers_human_m_virus_1MM_138_p.txt | Porcine adenovirus C, complete genome. |
| 139) | 18mers_human_m_virus_1MM_139_p.txt | Tupaia adenovirus 1, complete genome. |
| 140) | 18mers_human_m_virus_1MM_140_p.txt | Hemorrhagic enteritis virus, complete genome. |
| 141) | 18mers_human_m_virus_1MM_141_p.txt | Adoxophyes honmai nucleopolyhedrovirus, complete |
| | | genome. |
| 142) | 18mers_human_m_virus_1MM_142_p.txt | Autographa californica nucleopolyhedrovirus, complete |
| | | genome. |
| 143) | 18mers_human_m_virus_1MM_143_p.txt | Bombyx mori nuclear polyhedrosis virus, complete |
| | | genome. |
| 144) | 18mers_human_m_virus_1MM_144_p.txt | Choristoneura fumiferana MNPV, complete genome. |
| 145) | 18mers_human_m_virus_1MM_145_p.txt | Culex nigripalpus baculovirus, complete genome. |
| 146) | 18mers_human_m_virus_1MM_146_p.txt | Epiphyas postvittana nucleopolyhedrovirus, complete |
| | | genome. |
| 147) | 18mers_human_m_virus_1MM_147_p.txt | Helicoverpa armigera nuclear polyhedrosis virus, |
| | | complete genome. |
| 148) | 18mers_human_m_virus_1MM_148_p.txt | Helicoverpa zea single nucleocapsid |
| | | nucleopolyhedrovirus, complete genome. |
| 149) | 18mers_human_m_virus_1MM_149_p.txt | Heliocoverpa armigera nucleopolyhedrovirus G4, |
| | | complete genome. |
| 150) | 18mers_human_m_virus_1MM_150_p.txt | Mamestra configurata nucleopolyhedrovirus A, complete |
| | | genome. |
| 151) | 18mers_human_m_virus_1MM_151_p.txt | Mamestra configurata nucleopolyhedrovirus B, complete |
| | | genome. |
| 152) | 18mers_human_m_virus_1MM_152_p.txt | Orgyia pseudotsugata multicapsid nucleopolyhedrovirus, |
| | | complete genome. |
| 153) | 18mers_human_m_virus_1MM_153_p.txt | Plutella xylostella granulovirus, complete genome. |
| 154) | 18mers_human_m_virus_1MM_154_p.txt | Rachiplusia ou multiple nucleopolyhedrovirus, complete |
| | | genome. |
| 155) | 18mers_human_m_virus_1MM_155_p.txt | Spodoptera exigua nucleopolyhedrovirus, complete |
| | | genome. |
| 156) | 18mers_human_m_virus_1MM_156_p.txt | Xestia c-nigrum granulovirus, complete genome. |
| 157) | 18mers_human_m_virus_1MM_157_p.txt | Acyrthosiphon pisum bacteriophage APSE-1, complete |
| | | genome. |
| 158) | 18mers_human_m_virus_1MM_158_p.txt | Bacillus phage GA-1 virion, complete genome. |
| 159) | 18mers_human_m_virus_1MM_159_p.txt | Bacillus phage PZA, complete genome. |
| 160) | 18mers_human_m_virus_1MM_160_p.txt | Bacteriophage 933W provirus, complete genome. |
| 161) | 18mers_human_m_virus_1MM_161_p.txt | Bacteriophage A118 virion, complete genome. |
| 162) | 18mers_human_m_virus_1MM_162_p.txt | Bacteriophage B103, complete genome. |
| 163) | 18mers_human_m_virus_1MM_163_p.txt | Bacteriophage bIL170, complete genome. |
| 164) | 18mers_human_m_virus_1MM_164_p.txt | Bacteriophage bIL285, complete genome. |
| 165) | 18mers_human_m_virus_1MM_165_p.txt | Bacteriophage bIL286, complete genome. |
| 166) | 18mers_human_m_virus_1MM_166_p.txt | Bacteriophage bIL309, complete genome. |
| 167) | 18mers_human_m_virus_1MM_167_p.txt | Bacteriophage bIL310, complete genome. |
| 168) | 18mers_human_m_virus_1MM_168_p.txt | Bacteriophage bIL311, complete genome. |
| 169) | 18mers_human_m_virus_1MM_169_p.txt | Bacteriophage bIL312, complete genome. |
| 170) | 18mers_human_m_virus_1MM_170_p.txt | Bacteriophage HK620, complete genome. |
| 171) | 18mers_human_m_virus_1MM_171_p.txt | Bacteriophage HK97, complete genome. |
| 172) | 18mers_human_m_virus_1MM_172_p.txt | Bacteriophage K139, complete genome. |
| 173) | 18mers_human_m_virus_1MM_173_p.txt | Bacteriophage L-413C, complete genome. |
| 174) | 18mers_human_m_virus_1MM_174_p.txt | Bacteriophage lambda, complete genome. |
| 175) | 18mers_human_m_virus_1MM_175_p.txt | Bacteriophage N15 virion, complete genome. |
| 176) | 18mers_human_m_virus_1MM_176_p.txt | Bacteriophage P27, complete genome. |
| 177) | 18mers_human_m_virus_1MM_177_p.txt | Bacteriophage P4, complete genome. |
| 178) | 18mers_human_m_virus_1MM_178_p.txt | Bacteriophage phi CTX, complete genome. |
| 179) | 18mers_human_m_virus_1MM_179_p.txt | Bacteriophage phi ETA, complete genome. |
| 180) | 18mers_human_m_virus_1MM_180_p.txt | Bacteriophage phi-105, complete genome. |
| 181) | 18mers_human_m_virus_1MM_181_p.txt | Bacteriophage phi-BT1 virion, complete genome. |
| 182) | 18mers_human_m_virus_1MM_182_p.txt | Bacteriophage phi-C31, complete genome. |
| 183) | 18mers_human_m_virus_1MM_183_p.txt | Bacteriophage phi3626, complete genome. |
| 184) | 18mers_human_m_virus_1MM_184_p.txt | Bacteriophage phiE125, complete genome. |
| 185) | 18mers_human_m_virus_1MM_185_p.txt | Bacteriophage phig1e, complete genome. |
| 186) | 18mers_human_m_virus_1MM_186_p.txt | Bacteriophage phiYeO3-12, complete genome. |
| 187) | 18mers_human_m_virus_1MM_187_p.txt | Bacteriophage rlt, complete genome. |
| 188) | 18mers_human_m_virus_1MM_188_p.txt | Bacteriophage sk1, complete genome. |
| 189) | 18mers_human_m_virus_1MM_189_p.txt | Bacteriophage SPBc2, complete genome. |
| 190) | 18mers_human_m_virus_1MM_190_p.txt | Bacteriophage SPP1, complete genome. |
| 191) | 18mers_human_m_virus_1MM_191_p.txt | Bacteriophage T3, complete genome. |
| 192) | 18mers_human_m_virus_1MM_192_p.txt | Bacteriophage TP901-1, complete genome. |
| 193) | 18mers_human_m_virus_1MM_193_p.txt | Bacteriophage Tuc2009, complete genome. |
| 194) | 18mers_human_m_virus_1MM_194_p.txt | Bacteriophage VT2-Sa provirus, complete genome. |
| 195) | 18mers_human_m_virus_1MM_195_p.txt | Burkholderia cepacia phage Bcep781, complete genome. |
| 196) | 18mers_human_m_virus_1MM_196_p.txt | Enterobacteria phage 186, complete genome. |
| 197) | 18mers_human_m_virus_1MM_197_p.txt | Enterobacteria phage epsilon15, complete genome. |
| 198) | 18mers_human_m_virus_1MM_198_p.txt | Enterobacteria phage HK022 virion, complete genome. |
| 199) | 18mers_human_m_virus_1MM_199_p.txt | Enterobacteria phage Mu, complete genome. |
| 200) | 18mers_human_m_virus_1MM_200_p.txt | Enterobacteria phage P2, complete genome. |
| 201) | 18mers_human_m_virus_1MM_201_p.txt | Enterobacteria phage T4, complete genome. |
| 202) | 18mers_human_m_virus_1MM_202_p.txt | Enterobacteria phage T7. |
| 203) | 18mers_human_m_virus_1MM_203_p.txt | Haemophilus phage HP1, complete genome. |
| 204) | 18mers_human_m_virus_1MM_204_p.txt | Haemophilus phage HP2, complete genome. |
| 205) | 18mers_human_m_virus_1MM_205_p.txt | Lactobacillus bacteriophage phi adh, complete genome. |
| 206) | 18mers_human_m_virus_1MM_206_p.txt | Lactobacillus casei bacteriophage A2 virion, complete |
| | | genome. |
| 207) | 18mers_human_m_virus_1MM_207_p.txt | Lactococcus lactis bacteriophage u136, complete genome. |
| 208) | 18mers_human_m_virus_1MM_208_p.txt | Lactococcus phage BK5-T, complete genome. |
| 209) | 18mers_human_m_virus_1MM_209_p.txt | Lactococcus phage c2. |
| 210) | 18mers_human_m_virus_1MM_210_p.txt | Lactococcus phage P335, complete genome. |
| 211) | 18mers_human_m_virus_1MM_211_p.txt | Listeria phage 2389 virion, complete genome. |
| 212) | 18mers_human_m_virus_1MM_212_p.txt | Methanobacterium phage psiM2, complete genome. |
| 213) | 18mers_human_m_virus_1MM_213_p.txt | Methanothermobacter wolfeii prophage psiM100. |
| | | complete genome. |
| 214) | 18mers_human_m_virus_1MM_214_p.txt | Mycobacteriophage Bxb1, complete genome. |
| 215) | 18mers_human_m_virus_1MM_215_p.txt | Mycobacteriophage D29, complete genome. |
| 216) | 18mers_human_m_virus_1MM_216_p.txt | Mycobacteriophage TM4, complete genome. |
| 217) | 18mers_human_m_virus_1MM_217_p.txt | Mycobacterium phage L5. |
| 218) | 18mers_human_m_virus_1MM_218_p.txt | Mycoplasma virus P1, complete genome. |
| 219) | 18mers_human_m_virus_1MM_219_p.txt | Pseudomonas aeruginosa phage PaP3, complete genome. |
| 220) | 18mers_human_m_virus_1MM_220_p.txt | Pseudomonas phage D3, complete genome. |
| 221) | 18mers_human_m_virus_1MM_221_p.txt | Pseudomonas phage gh-1, complete genome. |
| 222) | 18mers_human_m_virus_1MM_222_p.txt | Pseudomonas phage phiKZ, complete genome. |
| 223) | 18mers_human_m_virus_1MM_223_p.txt | Roseophage SIO1, complete genome. |
| 224) | 18mers_human_m_virus_1MM_224_p.txt | Salmonella typhimurium bacteriophage ST64T virion, |
| | | complete genome. |
| 225) | 18mers_human_m_virus_1MM_225_p.txt | Salmonella typhimurium phage ST64B, complete |
| | | genome. |
| 226) | 18mers_human_m_virus_1MM_226_p.txt | Shigella flexneri bacteriophage V, complete genome. |
| 227) | 18mers_human_m_virus_1MM_227_p.txt | Staphylococcus aureus bacteriophage PVL provirus, |
| | | complete genome. |
| 228) | 18mers_human_m_virus_1MM_228_p.txt | Staphylococcus aureus phage phiP68, complete genome. |
| 229) | 18mers_human_m_virus_1MM_229_p.txt | Staphylococcus aureus prophage phiPV83 provirus, |
| | | complete genome. |
| 230) | 18mers_human_m_virus_1MM_230_p.txt | Staphylococcus aureus temperate phage phiSLT virion, |
| | | complete genome. |
| 231) | 18mers_human_m_virus_1MM_231_p.txt | Staphylococcus phage 44AHJD, complete genome. |
| 232) | 18mers_human_m_virus_1MM_232_p.txt | Streptococcus phage Cp-1, complete genome. |
| 233) | 18mers_human_m_virus_1MM_233_p.txt | Streptococcus thermophilus bacteriophage 7201, complete |
| | | genome. |
| 234) | 18mers_human_m_virus_1MM_234_p.txt | Streptococcus thermophilus bacteriophage DT1, complete |
| | | genome. |
| 235) | 18mers_human_m_virus_1MM_235_p.txt | Streptococcus thermophilus bacteriophage Sfi11, |
| | | complete genome. |
| 236) | 18mers_human_m_virus_1MM_236_p.txt | Streptococcus thermophilus bacteriophage Sfi19, |
| | | complete genome. |
| 237) | 18mers_human_m_virus_1MM_237_p.txt | Streptococcus thermophilus bacteriophage Sfi21, |
| | | complete genome. |
| 238) | 18mers_human_m_virus_1MM_238_p.txt | Streptococcus thermophilus temperate bacteriophage |
| | | O1205, complete genome. |
| 239) | 18mers_human_m_virus_1MM_239_p.txt | Stx2 converting bacteriophage 1 virion, complete genome. |
| 240) | 18mers_human_m_virus_1MM_240_p.txt | Temperate phage PhiN1H1.1, complete genome. |
| 241) | 18mers_human_m_virus_1MM_241_p.txt | Vibrio harveyi bacteriophage VHML, complete genome. |
| 242) | 18mers_human_m_virus_1MM_242_p.txt | Vibriophage VpV262, complete genome. |
| 243) | 18mers_human_m_virus_1MM_243_p.txt | Virus PhiCh1, complete genome. |
| 244) | 18mers_human_m_virus_1MM_244_p.txt | Yersinia pestis phage phiA1122, complete genome. |
| 245) | 18mers_human_m_virus_1MM_245_p.txt | Alteromonas phage PM2, complete genome. |
| 246) | 18mers_human_m_virus_1MM_246_p.txt | Sulfolobus virus 1, complete genome. |
| 247) | 18mers_human_m_virus_1MM_247_p.txt | Alcelaphine herpesvirus 1, complete genome. |
| 248) | 18mers_human_m_virus_1MM_248_p.txt | Ateline herpesvirus 3, complete genome. |
| 249) | 18mers_human_m_virus_1MM_249_p.txt | Bovine herpesvirus 1, complete genome. |
| 250) | 18mers_human_m_virus_1MM_250_p.txt | Bovine herpesvirus 4, complete genome. |
| 251) | 18mers_human_m_virus_1MM_251_p.txt | Callitrichine herpesvirus 3, complete genome. |
| 252) | 18mers_human_m_virus_1MM_252_p.txt | Cercopithecine herpesvirus 7, complete genome. |
| 253) | 18mers_human_m_virus_1MM_253_p.txt | Chimpanzee cytomegalovirus, complete genome. |
| 254) | 18mers_human_m_virus_1MM_254_p.txt | Equine herpesvirus 1, complete genome. |
| 255) | 18mers_human_m_virus_1MM_255_p.txt | Equine herpesvirus 2, complete genome. |
| 256) | 18mers_human_m_virus_1MM_256_p.txt | Equine herpesvirus 4, complete genome. |
| 257) | 18mers_human_m_virus_1MM_257_p.txt | Gallid herpesvirus 2, complete genome. |
| 258) | 18mers_human_m_virus_1MM_258_p.txt | Gallid herpesvirus 3, complete genome. |
| 259) | 18mers_human_m_virus_1MM_259_p.txt | Human herpesvirus 2, complete genome. |
| 260) | 18mers_human_m_virus_1MM_260_p.txt | Human herpesvirus 3, complete genome. |
| 261) | 18mers_human_m_virus_1MM_261_p.txt | Human herpesvirus 4, complete genome. |
| 262) | 18mers_human_m_virus_1MM_262_p.txt | Human herpesvirus 5, complete genome. |
| 263) | 18mers_human_m_virus_1MM_263_p.txt | Human herpesvirus 6, complete genome. |
| 264) | 18mers_human_m_virus_1MM_264_p.txt | Human herpesvirus 6B, complete genome. |
| 265) | 18mers_human_m_virus_1MM_265_p.txt | Human herpesvirus 7, complete genome. |
| 266) | 18mers_human_m_virus_1MM_266_p.txt | Human herpesvirus 8, genome. |
| 267) | 18mers_human_m_virus_1MM_267_p.txt | Ictalurid herpesvirus 1, complete genome. |
| 268) | 18mers_human_m_virus_1MM_268_p.txt | Meleagrid herpesvirus 1, complete genome. |
| 269) | 18mers_human_m_virus_1MM_269_p.txt | Mouse cytomegalovirus 1, complete genome. |
| 270) | 18mers_human_m_virus_1MM_270_p.txt | Murid herpesvirus 4, complete genome. |
| 271) | 18mers_human_m_virus_1MM_271_p.txt | Rat cytomegalovirus, complete genome. |
| 272) | 18mers_human_m_virus_1MM_272_p.txt | Saimiriine herpesvirus 2, complete genome. |
| 273) | 18mers_human_m_virus_1MM_273_p.txt | Tupaia herpesvirus, complete genome. |
| 274) | 18mers_human_m_virus_1MM_274_p.txt | Infectious spleen and kidney necrosis virus, complete |
| | | genome. |
| 275) | 18mers_human_m_virus_1MM_275_p.txt | Invertebrate iridescent virus 6, complete genome. |
| 276) | 18mers_human_m_virus_1MM_276_p.txt | Lymphocystis disease virus 1, complete genome. |
| 277) | 18mers_human_m_virus_1MM_277_p.txt | Rana tigrina ranavirus, complete genome. |
| 278) | 18mers_human_m_virus_1MM_278_p.txt | Sulfolobus islandicus filamentous virus, genome. |
| 279) | 18mers_human_m_virus_1MM_279_p.txt | Shrimp white spot syndrome virus, complete genome. |
| 280) | 18mers_human_m_virus_1MM_280_p.txt | Bovine papillomavirus, complete genome. |
| 281) | 18mers_human_m_virus_1MM_281_p.txt | Bovine papillomavirus type 2, complete genome. |
| 282) | 18mers_human_m_virus_1MM_282_p.txt | Bovine papillomavirus type 3, complete genome. |
| 283) | 18mers_human_m_virus_1MM_283_p.txt | Bovine papillomavirus type 4, complete genome. |
| 284) | 18mers_human_m_virus_1MM_284_p.txt | Bovine papillomavirus type 5, complete genome. |
| 285) | 18mers_human_m_virus_1MM_285_p.txt | Canine oral papillomavirus, complete genome. |
| 286) | 18mers_human_m_virus_1MM_286_p.txt | Common chimpanzee papillomavirus I, complete |
| | | genome. |
| 287) | 18mers_human_m_virus_1MM_287_p.txt | Cottontail rabbit papillomavirus, complete genome. |
| 288) | 18mers_human_m_virus_1MM_288_p.txt | Deer papillomavirus, complete genome. |
| 289) | 18mers_human_m_virus_1MM_289_p.txt | Equinus papillomavirus, complete genome. |
| 290) | 18mers_human_m_virus_1MM_290_p.txt | Equus caballus papillomavirus type 1, complete genome. |
| 291) | 18mers_human_m_virus_1MM_291_p.txt | European elk papillomavirus, complete genome. |
| 292) | 18mers_human_m_virus_1MM_292_p.txt | Feline papillomavirus, complete genome. |
| 293) | 18mers_human_m_virus_1MM_293_p.txt | Fringilla coelebs papillomavirus, complete genome. |
| 294) | 18mers_human_m_virus_1MM_294_p.txt | Hamster papovavirus, complete genome. |
| 295) | 18mers_human_m_virus_1MM_295_p.txt | Human papillomavirus candHPV85, complete genome. |
| 296) | 18mers_human_m_virus_1MM_296_p.txt | Human papillomavirus RTRX7, complete genome. |
| 297) | 18mers_human_m_virus_1MM_297_p.txt | Human papillomavirus type 10, complete genome. |
| 298) | 18mers_human_m_virus_1MM_298_p.txt | Human papillomavirus type 11, complete genome. |
| 299) | 18mers_human_m_virus_1MM_299_p.txt | Human papillomavirus type 12, complete genome. |
| 300) | 18mers_human_m_virus_1MM_300_p.txt | Human papillomavirus type 13, complete genome. |
| 301) | 18mers_human_m_virus_1MM_301_p.txt | Human papillomavirus type 14D, complete genome. |
| 302) | 18mers_human_m_virus_1MM_302_p.txt | Human papillomavirus type 15, complete genome. |
| 303) | 18mers_human_m_virus_1MM_303_p.txt | Human papillomavirus type 16, complete genome. |
| 304) | 18mers_human_m_virus_1MM_304_p.txt | Human papillomavirus type 17, complete genome. |
| 305) | 18mers_human_m_virus_1MM_305_p.txt | Human papillomavirus type 18, complete genome. |
| 306) | 18mers_human_m_virus_1MM_306_p.txt | Human papillomavirus type 19, complete genome. |
| 307) | 18mers_human_m_virus_1MM_307_p.txt | Human papillomavirus type 1a, complete genome. |
| 308) | 18mers_human_m_virus_1MM_308_p.txt | Human papillomavirus type 20, complete genome. |
| 309) | 18mers_human_m_virus_1MM_309_p.txt | Human papillomavirus type 21, complete genome. |
| 310) | 18mers_human_m_virus_1MM_310_p.txt | Human papillomavirus type 22, complete genome. |
| 311) | 18mers_human_m_virus_1MM_311_p.txt | Human papillomavirus type 23, complete genome. |
| 312) | 18mers_human_m_virus_1MM_312_p.txt | Human papillomavirus type 24, complete genome. |
| 313) | 18mers_human_m_virus_1MM_313_p.txt | Human papillomavirus type 25, complete genome. |
| 314) | 18mers_human_m_virus_1MM_314_p.txt | Human papillomavirus type 26, complete genome. |
| 315) | 18mers_human_m_virus_1MM_315_p.txt | Human papillomavirus type 27, complete genome. |
| 316) | 18mers_human_m_virus_1MM_316_p.txt | Human papillomavirus type 28, complete genome. |
| 317) | 18mers_human_m_virus_1MM_317_p.txt | Human papillomavirus type 29, complete genome. |
| 318) | 18mers_human_m_virus_1MM_318_p.txt | Human papillomavirus type 2a, complete genome. |
| 319) | 18mers_human_m_virus_1MM_319_p.txt | Human papillomavirus type 3, complete genome. |
| 320) | 18mers_human_m_virus_1MM_320_p.txt | Human papillomavirus type 30, complete genome. |
| 321) | 18mers_human_m_virus_1MM_321_p.txt | Human papillomavirus type 31, complete genome. |
| 322) | 18mers_human_m_virus_1MM_322_p.txt | Human papillomavirus type 32, complete genome. |
| 323) | 18mers_human_m_virus_1MM_323_p.txt | Human papillomavirus type 33, complete genome. |
| 324) | 18mers_human_m_virus_1MM_324_p.txt | Human papillomavirus type 34, complete genome. |
| 325) | 18mers_human_m_virus_1MM_325_p.txt | Human papillomavirus type 35, complete genome. |
| 326) | 18mers_human_m_virus_1MM_326_p.txt | Human papillomavirus type 36, complete genome. |
| 327) | 18mers_human_m_virus_1MM_327_p.txt | Human papillomavirus type 37, complete genome. |
| 328) | 18mers_human_m_virus_1MM_328_p.txt | Human papillomavirus type 38, complete genome. |
| 329) | 18mers_human_m_virus_1MM_329_p.txt | Human papillomavirus type 39, complete genome. |
| 330) | 18mers_human_m_virus_1MM_330_p.txt | Human papillomavirus type 4, complete genome. |
| 331) | 18mers_human_m_virus_1MM_331_p.txt | Human papillomavirus type 40, complete genome. |
| 332) | 18mers_human_m_virus_1MM_332_p.txt | Human papillomavirus type 41, complete genome. |
| 333) | 18mers_human_m_virus_1MM_333_p.txt | Human papillomavirus type 42, complete genome. |
| 334) | 18mers_human_m_virus_1MM_334_p.txt | Human papillomavirus type 44, complete genome. |
| 335) | 18mers_human_m_virus_1MM_335_p.txt | Human papillomavirus type 45, complete genome. |
| 336) | 18mers_human_m_virus_1MM_336_p.txt | Human papillomavirus type 47, complete genome. |
| 337) | 18mers_human_m_virus_1MM_337_p.txt | Human papillomavirus type 48, complete genome. |
| 338) | 18mers_human_m_virus_1MM_338_p.txt | Human papillomavirus type 49, complete genome. |
| 339) | 18mers_human_m_virus_1MM_339_p.txt | Human papillomavirus type 5, complete genome. |
| 340) | 18mers_human_m_virus_1MM_340_p.txt | Human papillomavirus type 50, complete genome. |
| 341) | 18mers_human_m_virus_1MM_341_p.txt | Human papillomavirus type 51, complete genome. |
| 342) | 18mers_human_m_virus_1MM_342_p.txt | Human papillomavirus type 52, complete genome. |
| 343) | 18mers_human_m_virus_1MM_343_p.txt | Human papillomavirus type 53, complete genome. |
| 344) | 18mers_human_m_virus_1MM_344_p.txt | Human papillomavirus type 54, complete genome. |
| 345) | 18mers_human_m_virus_1MM_345_p.txt | Human papillomavirus type 55, complete genome. |
| 346) | 18mers_human_m_virus_1MM_346_p.txt | Human papillomavirus type 56, complete genome. |
| 347) | 18mers_human_m_virus_1MM_347_p.txt | Human papillomavirus type 57, complete genome. |
| 348) | 18mers_human_m_virus_1MM_348_p.txt | Human papillomavirus type 58, complete genome. |
| 349) | 18mers_human_m_virus_1MM_349_p.txt | Human papillomavirus type 59, complete genome. |
| 350) | 18mers_human_m_virus_1MM_350_p.txt | Human papillomavirus type 5b, complete genome. |
| 351) | 18mers_human_m_virus_1MM_351_p.txt | Human papillomavirus type 6, complete genome. |
| 352) | 18mers_human_m_virus_1MM_352_p.txt | Human papillomavirus type 60, complete genome. |
| 353) | 18mers_human_m_virus_1MM_353_p.txt | Human papillomavirus type 61, complete genome. |
| 354) | 18mers_human_m_virus_1MM_354_p.txt | Human papillomavirus type 63, complete genome. |
| 355) | 18mers_human_m_virus_1MM_355_p.txt | Human papillomavirus type 65, complete genome. |
| 356) | 18mers_human_m_virus_1MM_356_p.txt | Human papillomavirus type 66, complete genome. |
| 357) | 18mers_human_m_virus_1MM_357_p.txt | Human papillomavirus type 67, complete genome. |
| 358) | 18mers_human_m_virus_1MM_358_p.txt | Human papillomavirus type 69, complete genome. |
| 359) | 18mers_human_m_virus_1MM_359_p.txt | Human papillomavirus type 6a, complete genome. |
| 360) | 18mers_human_m_virus_1MM_360_p.txt | Human papillomavirus type 6b, complete genome. |
| 361) | 18mers_human_m_virus_1MM_361_p.txt | Human papillomavirus type 7, complete genome. |
| 362) | 18mers_human_m_virus_1MM_362_p.txt | Human papillomavirus type 70, complete genome. |
| 363) | 18mers_human_m_virus_1MM_363_p.txt | Human papillomavirus type 71, complete genome. |
| 364) | 18mers_human_m_virus_1MM_364_p.txt | Human papillomavirus type 74, complete genome. |
| 365) | 18mers_human_m_virus_1MM_365_p.txt | Human papillomavirus type 8, complete genome. |
| 366) | 18mers_human_m_virus_1MM_366_p.txt | Human papillomavirus type 82, complete genome. |
| 367) | 18mers_human_m_virus_1MM_367_p.txt | Human papillomavirus type 83, complete genome. |
| 368) | 18mers_human_m_virus_1MM_368_p.txt | Human papillomavirus type 84, complete genome. |
| 369) | 18mers_human_m_virus_1MM_369_p.txt | Human papillomavirus type 86, complete genome. |
| 370) | 18mers_human_m_virus_1MM_370_p.txt | Human papillomavirus type 87, complete genome. |
| 371) | 18mers_human_m_virus_1MM_371_p.txt | Human papillomavirus type 89, complete genome. |
| 372) | 18mers_human_m_virus_1MM_372_p.txt | Human papillomavirus type 9, complete genome. |
| 373) | 18mers_human_m_virus_1MM_373_p.txt | Human papillomavirus type 90, complete genome. |
| 374) | 18mers_human_m_virus_1MM_374_p.txt | Human papillomavirus type 91, complete genome. |
| 375) | 18mers_human_m_virus_1MM_375_p.txt | Human papillomavirus type 92, complete genome. |
| 376) | 18mers_human_m_virus_1MM_376_p.txt | Monkey B-lymphotropic papovavirus, complete genome. |
| 377) | 18mers_human_m_virus_1MM_377_p.txt | Multimammate rat papillomavirus, complete genome. |
| 378) | 18mers_human_m_virus_1MM_378_p.txt | Ovine papillomavirus 1, complete genome. |
| 379) | 18mers_human_m_virus_1MM_379_p.txt | Ovine papillomavirus 2, complete genome. |
| 380) | 18mers_human_m_virus_1MM_380_p.txt | Phocoena spinipinnis papillomavirus, complete genome. |
| 381) | 18mers_human_m_virus_1MM_381_p.txt | Psittacus erithacus papillomavirus, complete genome. |
| 382) | 18mers_human_m_virus_1MM_382_p.txt | Rabbit oral papillomavirus, complete genome. |
| 383) | 18mers_human_m_virus_1MM_383_p.txt | Reindeer papillomavirus, complete genome. |
| 384) | 18mers_human_m_virus_1MM_384_p.txt | Rhesus monkey papillomavirus, complete genome. |
| 385) | 18mers_human_m_virus_1MM_385_p.txt | Ectocarpus siliculosus virus, complete genome. |
| 386) | 18mers_human_m_virus_1MM_386_p.txt | Paramecium bursaria Chlorella virus 1, complete genome. |
| 387) | 18mers_human_m_virus_1MM_387_p.txt | Acholeplasma phage L2, complete genome. |
| 388) | 18mers_human_m_virus_1MM_388_p.txt | African green monkey polyomavirus, complete genome. |
| 389) | 18mers_human_m_virus_1MM_389_p.txt | BK virus, complete genome. |
| 390) | 18mers_human_m_virus_1MM_390_p.txt | Bovine polyomavirus, complete genome. |
| 391) | 18mers_human_m_virus_1MM_391_p.txt | Budgerigar fledgling polyomavirus, complete genome. |
| 392) | 18mers_human_m_virus_1MM_392_p.txt | JC virus, complete genome. |
| 393) | 18mers_human_m_virus_1MM_393_p.txt | Murine pneumotropic virus, complete genome. |
| 394) | 18mers_human_m_virus_1MM_394_p.txt | Murine polyomavirus strain A2, complete genome. |
| 395) | 18mers_human_m_virus_1MM_395_p.txt | Simian virus 40, complete genome. |
| 396) | 18mers_human_m_virus_1MM_396_p.txt | Amsacta moorei entomopoxvirus, complete genome. |
| 397) | 18mers_human_m_virus_1MM_397_p.txt | Camelpox virus, complete genome. |
| 398) | 18mers_human_m_virus_1MM_398_p.txt | Cowpox virus, complete genome. |
| 399) | 18mers_human_m_virus_1MM_399_p.txt | Ectromelia virus, complete genome. |
| 400) | 18mers_human_m_virus_1MM_400_p.txt | Fowlpox virus, complete genome. |
| 401) | 18mers_human_m_virus_1MM_401_p.txt | Goatpox virus, complete genome. |
| 402) | 18mers_human_m_virus_1MM_402_p.txt | Lumpy skin disease virus, complete genome. |
| 403) | 18mers_human_m_virus_1MM_403_p.txt | Melanoplus sanguinipes entomopoxvirus, complete |
| | | genome. |
| 404) | 18mers_human_m_virus_1MM_404_p.txt | Monkeypox virus, complete genome. |
| 405) | 18mers_human_m_virus_1MM_405_p.txt | Myxoma virus, complete genome. |
| 406) | 18mers_human_m_virus_1MM_406_p.txt | Rabbit fibroma virus, complete genome. |
| 407) | 18mers_human_m_virus_1MM_407_p.txt | Sheeppox virus, complete genome. |
| 408) | 18mers_human_m_virus_1MM_408_p.txt | Swinepox virus, complete genome. |
| 409) | 18mers_human_m_virus_1MM_409_p.txt | Vaccinia virus, complete genome. |
| 410) | 18mers_human_m_virus_1MM_410_p.txt | Variola virus, complete genome. |
| 411) | 18mers_human_m_virus_1MM_411_p.txt | Yaba-like disease virus, complete genome. |
| 412) | 18mers_human_m_virus_1MM_412_p.txt | Sulfolobus virus SIRV-1, complete genome. |
| 413) | 18mers_human_m_virus_1MM_413_p.txt | Sulfolobus virus SIRV-2, complete genome. |
| 414) | 18mers_human_m_virus_1MM_414_p.txt | Enterobacteria phage PRD1, complete genome. |
| 415) | 18mers_human_m_virus_1MM_415_p.txt | Mycoplasma arthritidis bacteriophage MAV1, complete |
| | | genome. |
| 416) | 18mers_human_m_virus_1MM_416_p.txt | Drosophila x virus segment A, complete sequence. |
| 417) | 18mers_human_m_virus_1MM_418_p.txt | Infectious bursal disease virus segment A, complete |
| | | sequence. |
| 418) | 18mers_human_m_virus_1MM_420_p.txt | Infectious pancreatic necrosis virus segment A, complete |
| | | sequence. |
| 419) | 18mers_human_m_virus_1MM_422_p.txt | Marine birnavirus segment A, complete sequence. |
| 420) | 18mers_human_m_virus_1MM_424_p.txt | Bacteriophage phi-12 segment L, complete sequence. |
| 421) | 18mers_human_m_virus_1MM_427_p.txt | Bacteriophage phi-8 segment L, complete sequence. |
| 422) | 18mers_human_m_virus_1MM_430_p.txt | Pseudomonas bacteriophage phi-13 segment L, complete |
| | | sequence. |
| 423) | 18mers_human_m_virus_1MM_433_p.txt | Pseudomonas phage phi-6 segment L, complete sequence. |
| 424) | 18mers_human_m_virus_1MM_436_p.txt | Cryphonectria hypovirus, complete genome. |
| 425) | 18mers_human_m_virus_1MM_437_p.txt | Cryphonectria hypovirus 1, complete genome. |
| 426) | 18mers_human_m_virus_1MM_438_p.txt | Cryphonectria hypovirus 3, complete genome. |
| 427) | 18mers_human_m_virus_1MM_439_p.txt | Atkinsonella hypoxylon partitivirus RNA 1, complete |
| | | sequence. |
| 428) | 18mers_human_m_virus_1MM_442_p.txt | Discula destructiva virus 1 segment 1, complete sequence. |
| 429) | 18mers_human_m_virus_1MM_446_p.txt | Discula destructiva virus 2 segment 1, complete sequence. |
| 430) | 18mers_human_m_virus_1MM_448_p.txt | Fusarium poae virus 1 RNA 1, complete sequence. |
| 431) | 18mers_human_m_virus_1MM_450_p.txt | Gremmeniella abietina RNA virus MSI RNA 1, complete |
| | | sequence. |
| 432) | 18mers_human_m_virus_1MM_453_p.txt | Mycovirus FusoV RNA 1, complete sequence. |
| 433) | 18mers_human_m_virus_1MM_455_p.txt | Rhizoctonia solani virus RNA1, complete sequence. |
| 434) | 18mers_human_m_virus_1MM_457_p.txt | Banna virus segment 1, complete sequence. |
| 435) | 18mers_human_m_virus_1MM_469_p.txt | Bombyx mori cypovirus 1 segment 1, complete sequence. |
| 436) | 18mers_human_m_virus_1MM_479_p.txt | Colorado tick fever virus segment 1, complete sequence. |
| 437) | 18mers_human_m_virus_1MM_491_p.txt | Eyach virus segment 1, complete sequence. |
| 438) | 18mers_human_m_virus_1MM_503_p.txt | Kadipiro virus segment 1, complete sequence. |
| 439) | 18mers_human_m_virus_1MM_515_p.txt | Lymantria dispar cypovirus 1 segment 1, complete |
| | | sequence. |
| 440) | 18mers_human_m_virus_1MM_525_p.txt | Lymantria dispar cypovirus 14 segment 1, complete |
| | | sequence. |
| 441) | 18mers_human_m_virus_1MM_535_p.txt | Mammalian orthoreovirus 1 segment L1, complete |
| | | sequence. |
| 442) | 18mers_human_m_virus_1MM_545_p.txt | Mammalian orthoreovirus 2 segment L1, complete |
| | | sequence. |
| 443) | 18mers_human_m_virus_1MM_555_p.txt | Mammalian orthoreovirus 3 segment L1, complete |
| | | sequence. |
| 444) | 18mers_human_m_virus_1MM_565_p.txt | Nilaparvata lugens reovirus segment 1, complete |
| | | sequence. |
| 445) | 18mers_human_m_virus_1MM_575_p.txt | Rice black streaked dwarf virus segment 1, complete |
| | | sequence. |
| 446) | 18mers_human_m_virus_1MM_585_p.txt | Rice dwarf virus segment 1, complete sequence. |
| 447) | 18mers_human_m_virus_1MM_597_p.txt | Rice ragged stunt virus segment 1, complete sequence. |
| 448) | 18mers_human_m_virus_1MM_607_p.txt | Trichoplusia ni cytoplasmic polyhedrosis virus 15 |
| | | segment 1, complete |
| 449) | 18mers_human_m_virus_1MM_618_p.txt | Eimeria brunetti RNA virus 1, complete genome. |
| 450) | 18mers_human_m_virus_1MM_619_p.txt | Giardia lamblia virus, complete genome. |
| 451) | 18mers_human_m_virus_1MM_620_p.txt | Gremmeniella abietina RNA virus L1, complete genome. |
| 452) | 18mers_human_m_virus_1MM_621_p.txt | Helminthosporium victoriac virus 190S, complete |
| | | genome. |
| 453) | 18mers_human_m_virus_1MM_622_p.txt | Leishmania RNA virus 1-1, complete genome. |
| 454) | 18mers_human_m_virus_1MM_623_p.txt | Leishmania RNA virus 1-4, complete genome. |
| 455) | 18mers_human_m_virus_1MM_624_p.txt | Leishmania RNA virus 2-1, complete genome. |
| 456) | 18mers_human_m_virus_1MM_625_p.txt | Saccharomyces cerevisiae virus L-A, complete genome. |
| 457) | 18mers_human_m_virus_1MM_626_p.txt | Saccharomyces cerevisiae virus L-BC (La), complete |
| | | genome. |
| 458) | 18mers_human_m_virus_1MM_627_p.txt | Sphaeropsis sapinea RNA virus 1, complete genome. |
| 459) | 18mers_human_m_virus_1MM_628_p.txt | Sphaeropsis sapinea RNA virus 2, complete genome. |
| 460) | 18mers_human_m_virus_1MM_629_p.txt | Trichomonas vaginalis virus, complete genome. |
| 461) | 18mers_human_m_virus_1MM_630_p.txt | Trichomonas vaginalis virus 3, complete genome. |
| 462) | 18mers_human_m_virus_1MM_631_p.txt | Trichomonas vaginalis virus II, complete genome. |
| 463) | 18mers_human_m_virus_1MM_632_p.txt | Ustilago maydis virus H1, complete genome. |
| 464) | 18mers_human_m_virus_1MM_633_p.txt | Zygosaccharomyces bailii virus Z, complete genome. |
| 465) | 18mers_human_m_virus_1MM_634_p.txt | Diaporthe ambigua RNA virus 1, complete genome. |
| 466) | 18mers_human_m_virus_1MM_635_p.txt | Beak and feather disease virus, complete genome. |
| 467) | 18mers_human_m_virus_1MM_636_p.txt | Bovine circovirus, complete genome. |
| 468) | 18mers_human_m_virus_1MM_637_p.txt | Canary circovirus, complete genome. |
| 469) | 18mers_human_m_virus_1MM_638_p.txt | Chicken anemia virus, complete genome. |
| 470) | 18mers_human_m_virus_1MM_639_p.txt | Columbid circovirus, complete genome. |
| 471) | 18mers_human_m_virus_1MM_640_p.txt | Goose circovirus, complete genome. |
| 472) | 18mers_human_m_virus_1MM_641_p.txt | Porcine circovirus, complete genome. |
| 473) | 18mers_human_m_virus_1MM_642_p.txt | TT virus, complete genome. |
| 474) | 18mers_human_m_virus_1MM_643_p.txt | TTV-like mini virus, complete genome. |
| 475) | 18mers_human_m_virus_1MM_644_p.txt | Abutilon mosaic virus A component DNA, complete |
| | | sequence. |
| 476) | 18mers_human_m_virus_1MM_646_p.txt | African cassava mosaic virus DNA A, complete sequence. |
| 477) | 18mers_human_m_virus_1MM_648_p.txt | Ageratum enation virus, complete genome. |
| 478) | 18mers_human_m_virus_1MM_649_p.txt | Ageratum yellow vein China virus, complete genome. |
| 479) | 18mers_human_m_virus_1MM_650_p.txt | Ageratum yellow vein Sri Lanka virus segment A, |
| | | complete sequence. |
| 480) | 18mers_human_m_virus_1MM_651_p.txt | Ageratum yellow vein Taiwan virus, complete genome. |
| 481) | 18mers_human_m_virus_1MM_652_p.txt | Ageratum yellow vein virus, complete genome. |
| 482) | 18mers_human_m_virus_1MM_653_p.txt | Bean calico mosaic virus DNA A, complete sequence. |
| 483) | 18mers_human_m_virus_1MM_655_p.txt | Bean dwarf mosaic virus DNA A, complete sequence. |
| 484) | 18mers_human_m_virus_1MM_657_p.txt | Bean golden mosaic virus DNA A, complete sequence. |
| 485) | 18mers_human_m_virus_1MM_659_p.txt | Bean golden yellow mosaic virus DNA A, complete |
| | | sequence. |
| 486) | 18mers_human_m_virus_1MM_661_p.txt | Bean yellow dwarf virus, complete genome. |
| 487) | 18mers_human_m_virus_1MM_662_p.txt | Beet curly top virus, complete genome. |
| 488) | 18mers_human_m_virus_1MM_663_p.txt | Beet mild curly top virus, complete genome. |
| 489) | 18mers_human_m_virus_1MM_664_p.txt | Beet severe curly top virus, complete genome. |
| 490) | 18mers_human_m_virus_1MM_665_p.txt | Bhendi yellow vein mosaic virus, complete genome. |
| 491) | 18mers_human_m_virus_1MM_666_p.txt | Cabbage leaf curl virus DNA A, complete sequence. |
| 492) | 18mers_human_m_virus_1MM_668_p.txt | Chayote yellow mosaic virus, complete genome. |
| 493) | 18mers_human_m_virus_1MM_669_p.txt | Chilli leaf curl virus, complete genome. |
| 494) | 18mers_human_m_virus_1MM_670_p.txt | Chino del tomate virus DNA A, complete sequence. |
| 495) | 18mers_human_m_virus_1MM_672_p.txt | Chloris striate mosaic virus, complete genome. |
| 496) | 18mers_human_m_virus_1MM_673_p.txt | Cotton leaf crumple geminivirus DNA A, complete |
| | | sequence. |
| 497) | 18mers_human_m_virus_1MM_675_p.txt | Cotton leaf curl Alabad virus, complete genome. |
| 498) | 18mers_human_m_virus_1MM_676_p.txt | Cotton leaf curl Gezira virus, complete genome. |
| 499) | 18mers_human_m_virus_1MM_677_p.txt | Cotton leaf curl Kokhran virus, complete genome. |
| 500) | 18mers_human_m_virus_1MM_678_p.txt | Cotton leaf curl Multan virus, complete genome. |
| 501) | 18mers_human_m_virus_1MM_679_p.txt | Cotton leaf curl Rajasthan virus segment A, complete |
| | | sequence. |
| 502) | 18mers_human_m_virus_1MM_680_p.txt | Croton yellow vein mosaic virus, complete genome. |
| 503) | 18mers_human_m_virus_1MM_681_p.txt | Cucurbit leaf curl virus DNA A, complete sequence. |
| 504) | 18mers_human_m_virus_1MM_683_p.txt | Dicliptera yellow mottle virus DNA A, complete |
| | | sequence. |
| 505) | 18mers_human_m_virus_1MM_685_p.txt | Digitaria streak virus, complete genome. |
| 506) | 18mers_human_m_virus_1MM_686_p.txt | East African cassava mosaic Cameroon virus DNA A, |
| | | complete sequence. |
| 507) | 18mers_human_m_virus_1MM_688_p.txt | East African cassava mosaic virus DNA A, complete |
| | | sequence. |
| 508) | 18mers_human_m_virus_1MM_690_p.txt | East African cassava mosaic Zanzibar virus DNA A, |
| | | complete sequence. |
| 509) | 18mers_human_m_virus_1MM_692_p.txt | Egyptian sugarcane streak virus, complete genome. |
| 510) | 18mers_human_m_virus_1MM_693_p.txt | Eupatorium yellow vein virus, complete genome. |
| 511) | 18mers_human_m_virus_1MM_694_p.txt | Hollyhock leaf crumple virus, complete genome. |
| 512) | 18mers_human_m_virus_1MM_695_p.txt | Honeysuckle yellow vein mosaic virus, complete genome. |
| 513) | 18mers_human_m_virus_1MM_696_p.txt | Horseradish curly top virus, complete genome. |
| 514) | 18mers_human_m_virus_1MM_697_p.txt | Indian cassava mosaic virus DNA B, complete sequence. |
| 515) | 18mers_human_m_virus_1MM_699_p.txt | Ipomoea yellow vein virus, complete genome. |
| 516) | 18mers_human_m_virus_1MM_700_p.txt | Macroptilium mosaic Puerto Rico virus DNA A, complete |
| | | sequence. |
| 517) | 18mers_human_m_virus_1MM_702_p.txt | Macroptilium yellow mosaic Florida virus DNA A, |
| | | complete sequence. |
| 518) | 18mers_human_m_virus_1MM_704_p.txt | Macroptilium yellow mosaic virus, complete genome. |
| 519) | 18mers_human_m_virus_1MM_705_p.txt | Maize streak virus, complete genome. |
| 520) | 18mers_human_m_virus_1MM_706_p.txt | Malvastrum yellow vein virus, complete genome. |
| 521) | 18mers_human_m_virus_1MM_707_p.txt | Melon chlorotic leaf curl virus, complete genome. |
| 522) | 18mers_human_m_virus_1MM_708_p.txt | Miscanthus streak virus, complete genome. |
| 523) | 18mers_human_m_virus_1MM_709_p.txt | Mungbean yellow mosaic India virus DNA A, complete |
| | | sequence. |
| 524) | 18mers_human_m_virus_1MM_711_p.txt | Mungbean yellow mosaic virus DNA A, complete |
| | | sequence. |
| 525) | 18mers_human_m_virus_1MM_713_p.txt | Okra yellow vein mosaic virus, complete genome. |
| 526) | 18mers_human_m_virus_1MM_714_p.txt | Panicum streak virus, complete genome. |
| 527) | 18mers_human_m_virus_1MM_715_p.txt | Papaya leaf curl virus, complete genome. |
| 528) | 18mers_human_m_virus_1MM_716_p.txt | Pepper golden mosaic virus DNA A, complete sequence. |
| 529) | 18mers_human_m_virus_1MM_718_p.txt | Pepper huasteco yellow vein virus DNA A, complete |
| | | sequence. |
| 530) | 18mers_human_m_virus_1MM_720_p.txt | Pepper leaf curl Bangladesh virus segment A component, |
| | | complete sequence. |
| 531) | 18mers_human_m_virus_1MM_721_p.txt | Pepper leaf curl virus DNA A, complete sequence. |
| 532) | 18mers_human_m_virus_1MM_722_p.txt | Potato yellow mosaic Panama virus DNA A, complete |
| | | sequence. |
| 533) | 18mers_human_m_virus_1MM_724_p.txt | Potato yellow mosaic Trinidad virus DNA A, complete |
| | | sequence. |
| 534) | 18mers_human_m_virus_1MM_726_p.txt | Potato yellow mosaic virus DNA A, complete sequence. |
| 535) | 18mers_human_m_virus_1MM_728_p.txt | Rhynchosia Golden Mosaic Virus chromosome DNA A, |
| | | complete sequence. |
| 536) | 18mers_human_m_virus_1MM_729_p.txt | Sida golden mosaic Costa Rica virus DNA A, complete |
| | | sequence. |
| 537) | 18mers_human_m_virus_1MM_731_p.txt | Sida golden mosaic Florida virus, complete genome. |
| 538) | 18mers_human_m_virus_1MM_732_p.txt | Sida golden mosaic Honduras virus DNA A, complete |
| | | sequence. |
| 539) | 18mers_human_m_virus_1MM_734_p.txt | Sida golden mosaic virus DNA-A, complete sequence. |
| 540) | 18mers_human_m_virus_1MM_736_p.txt | Sida golden yellow vein virus, complete genome. |
| 541) | 18mers_human_m_virus_1MM_737_p.txt | Sida mottle virus, complete genome. |
| 542) | 18mers_human_m_virus_1MM_738_p.txt | Sida yellow mosaic virus, complete genome. |
| 543) | 18mers_human_m_virus_1MM_739_p.txt | Sida yellow vein virus DNA A, complete sequence. |
| 544) | 18mers_human_m_virus_1MM_741_p.txt | South African cassava mosaic virus DNA A, complete |
| | | sequence. |
| 545) | 18mers_human_m_virus_1MM_743_p.txt | Soybean crinkle leaf virus, complete genome. |
| 546) | 18mers_human_m_virus_1MM_744_p.txt | Squash leaf curl China virus, complete genome. |
| 547) | 18mers_human_m_virus_1MM_745_p.txt | Squash leaf curl virus A component DNA, complete |
| | | sequence. |
| 548) | 18mers_human_m_virus_1MM_747_p.txt | Squash leaf curl Yunnan virus, complete genome. |
| 549) | 18mers_human_m_virus_1MM_748_p.txt | Squash mild leaf curl virus DNA A, complete sequence. |
| 550) | 18mers_human_m_virus_1MM_750_p.txt | Squash yellow mild mottle virus DNA A, complete |
| | | sequence. |
| 551) | 18mers_human_m_virus_1MM_752_p.txt | Sri Lankan cassava mosaic virus DNA A, complete |
| | | sequence. |
| 552) | 18mers_human_m_virus_1MM_754_p.txt | Stachytarpheta leaf curl virus, complete genome. |
| 553) | 18mers_human_m_virus_1MM_755_p.txt | Sugarcane streak Reunion virus, complete genome. |
| 554) | 18mers_human_m_virus_1MM_756_p.txt | Sugarcane streak virus, complete genome. |
| 555) | 18mers_human_m_virus_1MM_757_p.txt | Sweet potato leaf curl Georgia virus, complete genome. |
| 556) | 18mers_human_m_virus_1MM_758_p.txt | Sweet potato leaf curl virus, complete genome. |
| 557) | 18mers_human_m_virus_1MM_759_p.txt | Tobacco curly shoot virus, complete genome. |
| 558) | 18mers_human_m_virus_1MM_760_p.txt | Tobacco leaf curl Japan virus, complete genome. |
| 559) | 18mers_human_m_virus_1MM_761_p.txt | Tobacco leaf curl Kochi virus, complete genome. |
| 560) | 18mers_human_m_virus_1MM_762_p.txt | Tobacco leaf curl Yamaguchi virus, complete genome. |
| 561) | 18mers_human_m_virus_1MM_763_p.txt | Tobacco leaf curl Yunnan virus, complete genome. |
| 562) | 18mers_human_m_virus_1MM_764_p.txt | Tobacco leaf curl Zimbabwe virus, complete genome. |
| 563) | 18mers_human_m_virus_1MM_765_p.txt | Tobacco yellow dwarf virus, complete genome. |
| 564) | 18mers_human_m_virus_1MM_766_p.txt | Tomato chlorotic mottle virus DNA A, complete |
| | | sequence. |
| 565) | 18mers_human_m_virus_1MM_768_p.txt | Tomato curly stunt virus, complete genome. |
| 566) | 18mers_human_m_virus_1MM_769_p.txt | Tomato golden mosaic virus DNA A, complete sequence. |
| 567) | 18mers_human_m_virus_1MM_771_p.txt | Tomato golden mottle virus, complete genome. |
| 568) | 18mers_human_m_virus_1MM_772_p.txt | Tomato leaf curl Bangalore virus, complete genome. |
| 569) | 18mers_human_m_virus_1MM_773_p.txt | Tomato leaf curl Bangladesh virus, complete genome. |
| 570) | 18mers_human_m_virus_1MM_774_p.txt | Tomato leaf curl Gujarat virus DNA A, complete |
| | | sequence. |
| 571) | 18mers_human_m_virus_1MM_776_p.txt | Tomato leaf curl Karnataka virus, complete genome. |
| 572) | 18mers_human_m_virus_1MM_777_p.txt | Tomato leaf curl Laos virus, complete genome. |
| 573) | 18mers_human_m_virus_1MM_778_p.txt | Tomato leaf curl Malaysia virus, complete genome. |
| 574) | 18mers_human_m_virus_1MM_779_p.txt | Tomato leaf curl New Delhi virus DNA A, complete |
| | | sequence. |
| 575) | 18mers_human_m_virus_1MM_781_p.txt | Tomato leaf curl Sri Lanka virus, complete genome. |
| 576) | 18mers_human_m_virus_1MM_782_p.txt | Tomato leaf curl Taiwan virus, complete genome. |
| 577) | 18mers_human_m_virus_1MM_783_p.txt | Tomato leaf curl Vietnam virus, complete genome. |
| 578) | 18mers_human_m_virus_1MM_784_p.txt | Tomato leaf curl virus, complete genome. |
| 579) | 18mers_human_m_virus_1MM_785_p.txt | Tomato mosaic Havana virus DNA A, complete sequence. |
| 580) | 18mers_human_m_virus_1MM_787_p.txt | Tomato mottle Taino virus DNA A, complete sequence. |
| 581) | 18mers_human_m_virus_1MM_789_p.txt | Tomato mottle virus DNA A, complete sequence. |
| 582) | 18mers_human_m_virus_1MM_791_p.txt | Tomato pseudo-curly top virus, complete genome. |
| 583) | 18mers_human_m_virus_1MM_792_p.txt | Tomato rugose mosaic virus DNA A, complete sequence. |
| 584) | 18mers_human_m_virus_1MM_794_p.txt | Tomato severe leaf curl virus, complete genome. |
| 585) | 18mers_human_m_virus_1MM_795_p.txt | Tomato yellow leaf curl China virus, complete genome. |
| 586) | 18mers_human_m_virus_1MM_796_p.txt | Tomato yellow leaf curl Gezira virus, complete genome. |
| 587) | 18mers_human_m_virus_1MM_797_p.txt | Tomato yellow leaf curl Malaga virus, complete genome. |
| 588) | 18mers_human_m_virus_1MM_798_p.txt | Tomato yellow leaf curl Sardinia virus, complete genome. |
| 589) | 18mers_human_m_virus_1MM_799_p.txt | Tomato yellow leaf curl Thailand virus DNA A, complete |
| | | sequence. |
| 590) | 18mers_human_m_virus_1MM_801_p.txt | Tomato yellow leaf curl virus, complete genome. |
| 591) | 18mers_human_m_virus_1MM_802_p.txt | Watermelon chlorotic stunt virus DNA A, complete |
| | | sequence. |
| 592) | 18mers_human_m_virus_1MM_804_p.txt | Acholeplasma phage MV-L1, complete genome. |
| 593) | 18mers_human_m_virus_1MM_805_p.txt | Bacteriophage VfO3K6, complete genome. |
| 594) | 18mers_human_m_virus_1MM_806_p.txt | Bacteriophage VfO4K68, complete genome. |
| 595) | 18mers_human_m_virus_1MM_807_p.txt | Bacteriophage VSKK, complete genome. |
| 596) | 18mers_human_m_virus_1MM_808_p.txt | Coliphage M13, complete genome. |
| 597) | 18mers_human_m_virus_1MM_809_p.txt | Enterobacteria phage l2-2. |
| 598) | 18mers_human_m_virus_1MM_810_p.txt | Enterobacteria phage lf1, complete genome. |
| 599) | 18mers_human_m_virus_1MM_811_p.txt | Enterobacteria phage lke. |
| 600) | 18mers_human_m_virus_1MM_812_p.txt | Propionibacterium phage phiB5, complete genome. |
| 601) | 18mers_human_m_virus_1MM_813_p.txt | Pseudomonas phage Pf1, complete genome. |
| 602) | 18mers_human_m_virus_1MM_814_p.txt | Pseudomonas phage Pf3, complete genome. |
| 603) | 18mers_human_m_virus_1MM_815_p.txt | Spiroplasma phage 1-C74, complete genome. |
| 604) | 18mers_human_m_virus_1MM_816_p.txt | Spiroplasma phage 1-R8A2B, complete genome. |
| 605) | 18mers_human_m_virus_1MM_817_p.txt | SVTS2 plectrovirus, complete genome. |
| 606) | 18mers_human_m_virus_1MM_818_p.txt | Vibrio cholerae filamentous bacteriophage fs-2, complete |
| | | genome. |
| 607) | 18mers_human_m_virus_1MM_819_p.txt | Vibrio cholerae O139 fs1 phage, complete genome. |
| 608) | 18mers_human_m_virus_1MM_820_p.txt | Vibrio cholerae phage VGJphi virion, complete genome. |
| 609) | 18mers_human_m_virus_1MM_821_p.txt | Vibrio phage VSK, complete genome. |
| 610) | 18mers_human_m_virus_1MM_822_p.txt | Xanthomonas phage Cf1c, complete genome. |
| 611) | 18mers_human_m_virus_1MM_823_p.txt | Chlamydia phage 2 virion, complete genome. |
| 612) | 18mers_human_m_virus_1MM_824_p.txt | Chlamydia phage phiCPAR39, complete genome. |
| 613) | 18mers_human_m_virus_1MM_825_p.txt | Chlamydia phage PhiCPG1, complete genome. |
| 614) | 18mers_human_m_virus_1MM_826_p.txt | Chlamydia psittaci bacteriophage chp1, complete genome. |
| 615) | 18mers_human_m_virus_1MM_827_p.txt | Coliphage alpha3, complete genome. |
| 616) | 18mers_human_m_virus_1MM_828_p.txt | Coliphage phiX174, complete genome. |
| 617) | 18mers_human_m_virus_1MM_829_p.txt | Enterobacteria phage G4, complete genome. |
| 618) | 18mers_human_m_virus_1MM_830_p.txt | Enterobacteria phage S13, complete genome. |
| 619) | 18mers_human_m_virus_1MM_831_p.txt | Phage phiMH2K virion, complete genome. |
| 620) | 18mers_human_m_virus_1MM_832_p.txt | Spiroplasma phage 4, complete genome. |
| 621) | 18mers_human_m_virus_1MM_833_p.txt | Ageratum yellow vein virus-associated nanovirus DNA, |
| | | complete genome. |
| 622) | 18mers_human_m_virus_1MM_834_p.txt | Banana bunchy top virus DNA 1, complete sequence. |
| 623) | 18mers_human_m_virus_1MM_840_p.txt | Coconut foliar decay virus, complete genome. |
| 624) | 18mers_human_m_virus_1MM_841_p.txt | Faba bean necrotic yellows virus DNA 1, complete |
| | | sequence. |
| 625) | 18mers_human_m_virus_1MM_851_p.txt | Milk vetch dwarf virus segment 1, complete sequence. |
| 626) | 18mers_human_m_virus_1MM_862_p.txt | Subterranean clover stunt virus DNA 1, complete |
| | | sequence. |
| 627) | 18mers_human_m_virus_1MM_870_p.txt | Acheta domesticus densovirus, complete genome. |
| 628) | 18mers_human_m_virus_1MM_871_p.txt | Adeno-associated virus 1, complete genome. |
| 629) | 18mers_human_m_virus_1MM_872_p.txt | Adeno-associated virus 2, complete genome. |
| 630) | 18mers_human_m_virus_1MM_873_p.txt | Adeno-associated virus 3, complete genome. |
| 631) | 18mers_human_m_virus_1MM_874_p.txt | Adeno-associated virus 3B, complete genome. |
| 632) | 18mers_human_m_virus_1MM_875_p.txt | Adeno-associated virus 4, complete genome. |
| 633) | 18mers_human_m_virus_1MM_876_p.txt | Adeno-associated virus 6, complete genome. |
| 634) | 18mers_human_m_virus_1MM_877_p.txt | Aedes albopictus densovirus, complete genome. |
| 635) | 18mers_human_m_virus_1MM_878_p.txt | Aleutian mink disease parvovirus, complete genome. |
| 636) | 18mers_human_m_virus_1MM_879_p.txt | B19 virus, complete genome. |
| 637) | 18mers_human_m_virus_1MM_880_p.txt | Bombyx mori densovirus 1, complete genome. |
| 638) | 18mers_human_m_virus_1MM_881_p.txt | Bombyx mori densovirus 5, complete genome. |
| 639) | 18mers_human_m_virus_1MM_882_p.txt | Bovine parvovirus, complete genome. |
| 640) | 18mers_human_m_virus_1MM_883_p.txt | Canine parvovirus, complete genome. |
| 641) | 18mers_human_m_virus_1MM_884_p.txt | Casphalia extranea densovirus, complete genome. |
| 642) | 18mers_human_m_virus_1MM_885_p.txt | Diatraea saccharalis densovirus, complete genome. |
| 643) | 18mers_human_m_virus_1MM_886_p.txt | Galleria mellonella densovirus, complete genome. |
| 644) | 18mers_human_m_virus_1MM_887_p.txt | Goose parvovirus, complete genome. |
| 645) | 18mers_human_m_virus_1MM_888_p.txt | Human erythrovirus V9, complete genome. |
| 646) | 18mers_human_m_virus_1MM_889_p.txt | Infectious hypodermal and hematopoietic necrosis virus, |
| | | complete genome. |
| 647) | 18mers_human_m_virus_1MM_890_p.txt | Junonia coenia densovirus, complete genome. |
| 648) | 18mers_human_m_virus_1MM_891_p.txt | LullI virus, complete genome. |
| 649) | 18mers_human_m_virus_1MM_892_p.txt | Mice minute virus, complete genome. |
| 650) | 18mers_human_m_virus_1MM_893_p.txt | Minute virus of canines, genome. |
| 651) | 18mers_human_m_virus_1MM_894_p.txt | Mouse parvovirus I, complete genome. |
| 652) | 18mers_human_m_virus_1MM_895_p.txt | Parvovirus H1, complete genome. |
| 653) | 18mers_human_m_virus_1MM_896_p.txt | Periplaneta fuliginosa densovirus, complete genome. |
| 654) | 18mers_human_m_virus_1MM_897_p.txt | Planococcus citri densovirus, complete genome. |
| 655) | 18mers_human_m_virus_1MM_898_p.txt | Porcine parvovirus, complete genome. |
| 656) | 18mers_human_m_virus_1MM_899_p.txt | Lassa virus segment L, complete sequence. |
| 657) | 18mers_human_m_virus_1MM_901_p.txt | Lymphocytic choriomeningitis virus segment L, complete |
| | | sequence. |
| 658) | 18mers_human_m_virus_1MM_903_p.txt | Tacaribe virus segment L, complete sequence. |
| 659) | 18mers_human_m_virus_1MM_905_p.txt | Andes virus segment L, complete sequence. |
| 660) | 18mers_human_m_virus_1MM_908_p.txt | Bunyamwera virus L segment, complete sequence. |
| 661) | 18mers_human_m_virus_1MM_911_p.txt | Dugbe virus segment L, complete sequence. |
| 662) | 18mers_human_m_virus_1MM_914_p.txt | Groundnut bud necrosis virus segment L, complete |
| | | sequence. |
| 663) | 18mers_human_m_virus_1MM_917_p.txt | Impatiens necrotic spot virus segment L, complete |
| | | sequence. |
| 664) | 18mers_human_m_virus_1MM_920_p.txt | La Crosse virus segment L, complete sequence. |
| 665) | 18mers_human_m_virus_1MM_923_p.txt | Rift Valley fever virus, complete genome. |
| 666) | 18mers_human_m_virus_1MM_926_p.txt | Tomato spotted wilt virus RNA L, complete sequence. |
| 667) | 18mers_human_m_virus_1MM_929_p.txt | Watermelon spotted wilt virus segment L, complete |
| | | sequence. |
| 668) | 18mers_human_m_virus_1MM_932_p.txt | Australian bat lyssavirus, complete genome. |
| 669) | 18mers_human_m_virus_1MM_933_p.txt | Avian paramyxovirus 6, complete genome. |
| 670) | 18mers_human_m_virus_1MM_934_p.txt | Boma disease virus, complete genome. |
| 671) | 18mers_human_m_virus_1MM_935_p.txt | Bovine ephemeral fever virus, complete genome. |
| 672) | 18mers_human_m_virus_1MM_936_p.txt | Bovine parainfluenza virus 3, complete genome. |
| 673) | 18mers_human_m_virus_1MM_937_p.txt | Canine distemper virus, complete genome. |
| 674) | 18mers_human_m_virus_1MM_938_p.txt | Hendra virus, complete genome. |
| 675) | 18mers_human_m_virus_1MM_939_p.txt | Human metapneumovirus, genome. |
| 676) | 18mers_human_m_virus_1MM_940_p.txt | Human parainfluenza virus 1 strain Washington/1964, |
| | | complete genome. |
| 677) | 18mers_human_m_virus_1MM_941_p.txt | Human parainfluenza virus 2, complete genome. |
| 678) | 18mers_human_m_virus_1MM_942_p.txt | Human parainfluenza virus 3, complete genome. |
| 679) | 18mers_human_m_virus_1MM_943_p.txt | Human respiratory syncytial virus, complete genome. |
| 680) | 18mers_human_m_virus_1MM_944_p.txt | Infectious hematopoietic necrosis virus, complete |
| | | genome. |
| 681) | 18mers_human_m_virus_1MM_945_p.txt | Marburg virus, complete genome. |
| 682) | 18mers_human_m_virus_1MM_946_p.txt | Measles virus, complete genome. |
| 683) | 18mers_human_m_virus_1MM_947_p.txt | Mumps virus, complete genome. |
| 684) | 18mers_human_m_virus_1MM_948_p.txt | Newcastle disease virus, complete genome. |
| 685) | 18mers_human_m_virus_1MM_949_p.txt | Nipah virus, complete genome. |
| 686) | 18mers_human_m_virus_1MM_950_p.txt | Northern cereal mosaic virus, complete genome. |
| 687) | 18mers_human_m_virus_1MM_951_p.txt | Rabies virus, complete genome. |
| 688) | 18mers_human_m_virus_1MM_952_p.txt | Respiratory syncytial virus, complete genome. |
| 689) | 18mers_human_m_virus_1MM_953_p.txt | Reston Ebola virus, complete genome. |
| 690) | 18mers_human_m_virus_1MM_954_p.txt | Rice yellow stunt virus, complete genome. |
| 691) | 18mers_human_m_virus_1MM_955_p.txt | Sendai virus, complete genome. |
| 692) | 18mers_human_m_virus_1MM_956_p.txt | Snakehead rhabdovirus, complete genome. |
| 693) | 18mers_human_m_virus_1MM_957_p.txt | Sonchus yellow net virus, complete genome. |
| 694) | 18mers_human_m_virus_1MM_958_p.txt | Spring viremia of carp virus, complete genome. |
| 695) | 18mers_human_m_virus_1MM_959_p.txt | Tioman virus, complete genome. |
| 696) | 18mers_human_m_virus_1MM_960_p.txt | Tupaia paramyxovirus, complete genome. |
| 697) | 18mers_human_m_virus_1MM_961_p.txt | Vesicular stomatitis Indiana virus, complete genome. |
| 698) | 18mers_human_m_virus_1MM_962_p.txt | Viral hemorrhagic septicemia virus, complete genome. |
| 699) | 18mers_human_m_virus_1MM_963_p.txt | Zaire Ebola virus, complete genome. |
| 700) | 18mers_human_m_virus_1MM_964_p.txt | Mirafiori lettuce virus RNA 1, complete sequence. |
| 701) | 18mers_human_m_virus_1MM_968_p.txt | Influenza A virus RNA segment 1, complete sequence. |
| 702) | 18mers_human_m_virus_1MM_976_p.txt | Influenza A virus (A/Puerto Rico/8/34/Mount |
| | | Sinai(H1N1)) segment 1, complete |
| 703) | 18mers_human_m_virus_1MM_984_p.txt | Influenza B virus RNA-1, complete sequence. |
| 704) | 18mers_human_m_virus_1MM_992_p.txt | Influenza B virus (B/Memphis/12/97) segment 1, |
| | | complete sequence. |
| 705) | 18mers_human_m_virus_1MM_1000_p.txt | Influenza B virus (B/Memphis/12/97-MA) segment 1, |
| | | complete sequence. |
| 706) | 18mers_human_m_virus_1MM_1008_p.txt | Rice grassy stunt virus RNA 1, complete sequence. |
| 707) | 18mers_human_m_virus_1MM_1014_p.txt | Rice stripe virus RNA 1, complete sequence. |
| 708) | 18mers_human_m_virus_1MM_1018_p.txt | Garlic virus C, complete genome. |
| 709) | 18mers_human_m_virus_1MM_1019_p.txt | Garlic virus E, complete genome. |
| 710) | 18mers_human_m_virus_1MM_1020_p.txt | Garlic virus X, complete genome. |
| 711) | 18mers_human_m_virus_1MM_1021_p.txt | Shallot virus X, complete genome. |
| 712) | 18mers_human_m_virus_1MM_1022_p.txt | Avian nephritis virus, complete genome. |
| 713) | 18mers_human_m_virus_1MM_1023_p.txt | Human astrovirus, complete genome. |
| 714) | 18mers_human_m_virus_1MM_1024_p.txt | Mink astrovirus, complete genome. |
| 715) | 18mers_human_m_virus_1MM_1025_p.txt | Ovine astrovirus, complete genome. |
| 716) | 18mers_human_m_virus_1MM_1026_p.txt | Turkey astrovirus, complete genome. |
| 717) | 18mers_human_m_virus_1MM_1027_p.txt | Mushroom bacilliform virus, complete genome. |
| 718) | 18mers_human_m_virus_1MM_1028_p.txt | Beet necrotic yellow vein virus RNA 1, complete |
| | | sequence. |
| 719) | 18mers_human_m_virus_1MM_1033_p.txt | Beet soil-borne mosaic virus RNA1, complete sequence. |
| 720) | 18mers_human_m_virus_1MM_1037_p.txt | Alfalfa mosaic virus RNA 1, complete sequence. |
| 721) | 18mers_human_m_virus_1MM_1040_p.txt | American plum line pattern virus RNA1, complete |
| | | sequence. |
| 722) | 18mers_human_m_virus_1MM_1043_p.txt | Apple mosaic virus RNA 1, complete sequence. |
| 723) | 18mers_human_m_virus_1MM_1046_p.txt | Broad bean mottle virus RNA 1, complete sequence. |
| 724) | 18mers_human_m_virus_1MM_1049_p.txt | Brome mosaic virus RNA 1, complete sequence. |
| 725) | 18mers_human_m_virus_1MM_1052_p.txt | Citrus leaf rugose virus RNA 1, complete sequence. |
| 726) | 18mers_human_m_virus_1MM_1055_p.txt | Cowpea chlorotic mottle virus RNA 1, complete |
| | | sequence. |
| 727) | 18mers_human_m_virus_1MM_1058_p.txt | Cucumber mosaic virus RNA 1, complete sequence. |
| 728) | 18mers_human_m_virus_1MM_1061_p.txt | Elm mottle virus RNA 1, complete sequence. |
| 729) | 18mers_human_m_virus_1MM_1064_p.txt | Olive latent virus 2 RNA 1, complete sequence. |
| 730) | 18mers_human_m_virus_1MM_1067_p.txt | Peanut stunt virus RNA 1, complete sequence. |
| 731) | 18mers_human_m_virus_1MM_1070_p.txt | Pelargonium zonate spot virus RNA 1, complete |
| | | sequence. |
| 732) | 18mers_human_m_virus_1MM_1073_p.txt | Prunus necrotic ringspot virus RNA1, complete sequence. |
| 733) | 18mers_human_m_virus_1MM_1076_p.txt | Spinach latent virus RNA 1, complete sequence. |
| 734) | 18mers_human_m_virus_1MM_1079_p.txt | Spring beauty latent virus RNA 1, complete sequence. |
| 735) | 18mers_human_m_virus_1MM_1082_p.txt | Tobacco streak virus RNA 1, complete sequence. |
| 736) | 18mers_human_m_virus_1MM_1085_p.txt | Tomato aspermy virus RNA 1, complete sequence. |
| 737) | 18mers_human_m_virus_1MM_1088_p.txt | Tulare apple mosaic virus RNA1, complete sequence. |
| 738) | 18mers_human_m_virus_1MM_1091_p.txt | Calicivirus strain NB, complete genome. |
| 739) | 18mers_human_m_virus_1MM_1092_p.txt | Canine calicivirus, complete genome. |
| 740) | 18mers_human_m_virus_1MM_1093_p.txt | European brown hare syndrome virus, complete genome. |
| 741) | 18mers_human_m_virus_1MM_1094_p.txt | Feline calicivirus, complete genome. |
| 742) | 18mers_human_m_virus_1MM_1095_p.txt | Norwalk virus, complete genome. |
| 743) | 18mers_human_m_virus_1MM_1096_p.txt | Porcine enteric calicivirus, complete genome. |
| 744) | 18mers_human_m_virus_1MM_1097_p.txt | Rabbit hemorrhagic disease virus, complete genome. |
| 745) | 18mers_human_m_virus_1MM_1098_p.txt | Vesicular exanthema of swine virus, complete genome. |
| 746) | 18mers_human_m_virus_1MM_1099_p.txt | Walrus calicivirus, complete genome. |
| 747) | 18mers_human_m_virus_1MM_1100_p.txt | Apple stem grooving virus, complete genome. |
| 748) | 18mers_human_m_virus_1MM_1101_p.txt | Cherry virus A, complete genome. |
| 749) | 18mers_human_m_virus_1MM_1102_p.txt | Aconitum latent virus, complete genome. |
| 750) | 18mers_human_m_virus_1MM_1103_p.txt | Blueberry scorch virus, complete genome. |
| 751) | 18mers_human_m_virus_1MM_1104_p.txt | Garlic latent virus, complete genome. |
| 752) | 18mers_human_m_virus_1MM_1105_p.txt | Hop latent virus, complete genome. |
| 753) | 18mers_human_m_virus_1MM_1106_p.txt | Potato virus M, complete genome. |
| 754) | 18mers_human_m_virus_1MM_1107_p.txt | Beet yellows virus, complete genome. |
| 755) | 18mers_human_m_virus_1MM_1108_p.txt | Citrus tristeza virus, complete genome. |
| 756) | 18mers_human_m_virus_1MM_1109_p.txt | Cucumber yellows virus RNA 1, complete sequence. |
| 757) | 18mers_human_m_virus_1MM_1111_p.txt | Grapevine leafroll-associated virus 3, complete genome. |
| 758) | 18mers_human_m_virus_1MM_1112_p.txt | Grapevine rootstock stem lesion associated virus, |
| | | complete genome. |
| 759) | 18mers_human_m_virus_1MM_1113_p.txt | Lettuce infectious yellows virus RNA 1, complete |
| | | sequence. |
| 760) | 18mers_human_m_virus_1MM_1115_p.txt | Little cherry virus 1, complete genome. |
| 761) | 18mers_human_m_virus_1MM_1116_p.txt | Sweet potato chlorotic stunt virus RNA 1, complete |
| | | sequence. |
| 762) | 18mers_human_m_virus_1MM_1118_p.txt | Bean pod mottle virus RNA1, complete sequence. |
| 763) | 18mers_human_m_virus_1MM_1120_p.txt | Beet ringspot virus RNA 1, complete sequence. |
| 764) | 18mers_human_m_virus_1MM_1122_p.txt | Blackcurrant reversion virus RNA1, complete sequence. |
| 765) | 18mers_human_m_virus_1MM_1124_p.txt | Broad bean wilt virus 2 RNA1, complete sequence. |
| 766) | 18mers_human_m_virus_1MM_1126_p.txt | Cowpea mosaic virus RNA 1, complete sequence. |
| 767) | 18mers_human_m_virus_1MM_1128_p.txt | Cowpea severe mosaic virus RNA 1, complete sequence. |
| 768) | 18mers_human_m_virus_1MM_1130_p.txt | Cycas necrotic stunt virus RNA 1, complete sequence. |
| 769) | 18mers_human_m_virus_1MM_1132_p.txt | Grapevine chrome mosaic virus RNA 1, complete |
| | | sequence. |
| 770) | 18mers_human_m_virus_1MM_1134_p.txt | Grapevine fanleaf virus RNA 1, complete sequence. |
| 771) | 18mers_human_m_virus_1MM_1136_p.txt | Patchouli mild mosaic virus RNA 1, complete sequence. |
| 772) | 18mers_human_m_virus_1MM_1138_p.txt | Red clover mottle virus RNA 1, complete sequence. |
| 773) | 18mers_human_m_virus_1MM_1140_p.txt | Squash mosaic virus RNA 1, complete sequence. |
| 774) | 18mers_human_m_virus_1MM_1142_p.txt | Tomato black ring virus RNA 1, complete sequence. |
| 775) | 18mers_human_m_virus_1MM_1144_p.txt | Tomato ringspot virus RNA 1, complete sequence. |
| 776) | 18mers_human_m_virus_1MM_1146_p.txt | Acute bee paralysis virus, complete genome. |
| 777) | 18mers_human_m_virus_1MM_1147_p.txt | Aphid lethal paralysis virus, complete genome. |
| 778) | 18mers_human_m_virus_1MM_1148_p.txt | Black queen cell virus, complete genome. |
| 779) | 18mers_human_m_virus_1MM_1149_p.txt | Cricket paralysis virus, complete genome. |
| 780) | 18mers_human_m_virus_1MM_1150_p.txt | Drosophila C virus, complete genome. |
| 781) | 18mers_human_m_virus_1MM_1151_p.txt | Himetobi P virus, complete genome. |
| 782) | 18mers_human_m_virus_1MM_1152_p.txt | Plautia stali intestine virus, complete genome. |
| 783) | 18mers_human_m_virus_1MM_1153_p.txt | Rhopalosiphum padi virus, complete genome. |
| 784) | 18mers_human_m_virus_1MM_1154_p.txt | Taura syndrome virus, complete genome. |
| 785) | 18mers_human_m_virus_1MM_1155_p.txt | Triatoma virus, complete genome. |
| 786) | 18mers_human_m_virus_1MM_1156_p.txt | Alkhurma virus, complete genome. |
| 787) | 18mers_human_m_virus_1MM_1157_p.txt | Apoi virus, genome. |
| 788) | 18mers_human_m_virus_1MM_1158_p.txt | Bovine viral diarrhea virus genotype 2, complete genome. |
| 789) | 18mers_human_m_virus_1MM_1159_p.txt | Cell fusing agent virus, complete genome. |
| 790) | 18mers_human_m_virus_1MM_1160_p.txt | Deer tick virus, genome. |
| 791) | 18mers_human_m_virus_1MM_1161_p.txt | Dengue virus, complete genome. |
| 792) | 18mers_human_m_virus_1MM_1162_p.txt | Hepatitis C virus, complete genome. |
| 793) | 18mers_human_m_virus_1MM_1163_p.txt | Hepatitis G virus, complete genome. |
| 794) | 18mers_human_m_virus_1MM_1164_p.txt | Hepatitis GB virus A, complete genome. |
| 795) | 18mers_human_m_virus_1MM_1165_p.txt | Hepatitis GB virus B, complete genome. |
| 796) | 18mers_human_m_virus_1MM_1166_p.txt | Hepatitis GB virus C, complete genome. |
| 797) | 18mers_human_m_virus_1MM_1167_p.txt | Japanese encephalitis virus, genome. |
| 798) | 18mers_human_m_virus_1MM_1168_p.txt | Langat virus, complete genome. |
| 799) | 18mers_human_m_virus_1MM_1169_p.txt | Louping ill virus, complete genome. |
| 800) | 18mers_human_m_virus_1MM_1170_p.txt | Modoc virus, genome. |
| 801) | 18mers_human_m_virus_1MM_1171_p.txt | Montana myotis leukoencephalitis virus, complete |
| | | genome. |
| 802) | 18mers_human_m_virus_1MM_1172_p.txt | Murray Valley encephalitis virus, complete genome. |
| 803) | 18mers_human_m_virus_1MM_1173_p.txt | Pestivirus Giraffe-1, complete genome. |
| 804) | 18mers_human_m_virus_1MM_1174_p.txt | Pestivirus Reindeer-1, complete genome. |
| 805) | 18mers_human_m_virus_1MM_1175_p.txt | Pestivirus type 2, complete genome. |
| 806) | 18mers_human_m_virus_1MM_1176_p.txt | Pestivirus type 3, complete genome. |
| 807) | 18mers_human_m_virus_1MM_1177_p.txt | Powassan virus, complete genome. |
| 808) | 18mers_human_m_virus_1MM_1178_p.txt | Rio Bravo virus, genome. |
| 809) | 18mers_human_m_virus_1MM_1179_p.txt | Tamana bat virus, genome. |
| 810) | 18mers_human_m_virus_1MM_1180_p.txt | Tick-borne encephalitis virus, complete genome. |
| 811) | 18mers_human_m_virus_1MM_1181_p.txt | West Nile virus, complete genome. |
| 812) | 18mers_human_m_virus_1MM_1182_p.txt | Yellow fever virus, complete genome. |
| 813) | 18mers_human_m_virus_1MM_1183_p.txt | Apple stem pitting virus, complete genome. |
| 814) | 18mers_human_m_virus_1MM_1184_p.txt | Banana mild mosaic virus, complete genome. |
| 815) | 18mers_human_m_virus_1MM_1185_p.txt | Cherry green ring mottle virus, complete genome. |
| 816) | 18mers_human_m_virus_1MM_1186_p.txt | Cherry necrotic rusty mottle virus, complete genome. |
| 817) | 18mers_human_m_virus_1MM_1187_p.txt | Rupestris stem pitting associated virus-1, complete |
| | | genome. |
| 818) | 18mers_human_m_virus_1MM_1188_p.txt | Chinese wheat mosaic virus RNA1, complete sequence. |
| 819) | 18mers_human_m_virus_1MM_1190_p.txt | Oat golden stripe virus RNA 1, complete sequence. |
| 820) | 18mers_human_m_virus_1MM_1192_p.txt | Soil-borne cereal mosaic virus RNA1, complete sequence. |
| 821) | 18mers_human_m_virus_1MM_1194_p.txt | Soil-borne wheat mosaic virus RNA1, complete sequence. |
| 822) | 18mers_human_m_virus_1MM_1196_p.txt | Sorghum chlorotic spot virus RNA 1, complete sequence. |
| 823) | 18mers_human_m_virus_1MM_1198_p.txt | Hepatitis E virus, complete genome. |
| 824) | 18mers_human_m_virus_1MM_1199_p.txt | Barley stripe mosaic virus RNA 1, complete sequence. |
| 825) | 18mers_human_m_virus_1MM_1202_p.txt | Raspberry bushy dwarf virus RNA 1, complete sequence. |
| 826) | 18mers_human_m_virus_1MM_1204_p.txt | Infectious flacherie virus, complete genome. |
| 827) | 18mers_human_m_virus_1MM_1205_p.txt | Perina nuda picorna-like virus, complete genome. |
| 828) | 18mers_human_m_virus_1MM_1206_p.txt | Sacbrood virus, complete genome. |
| 829) | 18mers_human_m_virus_1MM_1207_p.txt | Bacteriophage AP205 virion, complete genome. |
| 830) | 18mers_human_m_virus_1MM_1208_p.txt | Bacteriophage M11, complete genome. |
| 831) | 18mers_human_m_virus_1MM_1209_p.txt | Bacteriophage SP, complete genome. |
| 832) | 18mers_human_m_virus_1MM_1210_p.txt | Enterobacteria phage fr, complete genome. |
| 833) | 18mers_human_m_virus_1MM_1211_p.txt | Enterobacteria phage GA, complete genome. |
| 834) | 18mers_human_m_virus_1MM_1212_p.txt | Enterobacteria phage KU1, complete genome. |
| 835) | 18mers_human_m_virus_1MM_1213_p.txt | Enterobacteria phage MX1. |
| 836) | 18mers_human_m_virus_1MM_1214_p.txt | Enterobacteria phage NL95. |
| 837) | 18mers_human_m_virus_1MM_1215_p.txt | Enterobacterio phage MS2, complete genome. |
| 838) | 18mers_human_m_virus_1MM_1216_p.txt | Pseudomonas phage PP7, complete genome. |
| 839) | 18mers_human_m_virus_1MM_1217_p.txt | Barley yellow dwarf virus—GAV, complete genome. |
| 840) | 18mers_human_m_virus_1MM_1218_p.txt | Barley yellow dwarf virus—MAV, complete genome. |
| 841) | 18mers_human_m_virus_1MM_1219_p.txt | Barley yellow dwarf virus—PAV, complete genome. |
| 842) | 18mers_human_m_virus_1MM_1220_p.txt | Barley yellow dwarf virus—PAS, complete genome. |
| 843) | 18mers_human_m_virus_1MM_1221_p.txt | Bean leafroll virus, complete genome. |
| 844) | 18mers_human_m_virus_1MM_1222_p.txt | Beet chlorosis virus, complete genome. |
| 845) | 18mers_human_m_virus_1MM_1223_p.txt | Beet mild yellowing virus, complete genome. |
| 846) | 18mers_human_m_virus_1MM_1224_p.txt | Beet western yellows virus, complete genome. |
| 847) | 18mers_human_m_virus_1MM_1225_p.txt | Cereal yellow dwarf virus—RPS, complete genome. |
| 848) | 18mers_human_m_virus_1MM_1226_p.txt | Cereal yellow dwarf virus—RPV, complete genome. |
| 849) | 18mers_human_m_virus_1MM_1227_p.txt | Cucurbit aphid-borne yellows virus, complete genome. |
| 850) | 18mers_human_m_virus_1MM_1228_p.txt | Pea enation mosaic virus-1, complete genome. |
| 851) | 18mers_human_m_virus_1MM_1229_p.txt | Potato leafroll virus, complete genome. |
| 852) | 18mers_human_m_virus_1MM_1230_p.txt | Soybean dwarf virus, complete genome. |
| 853) | 18mers_human_m_virus_1MM_1231_p.txt | Sugarcane yellow leaf virus, complete genome. |
| 854) | 18mers_human_m_virus_1MM_1232_p.txt | Turnip yellows virus, complete genome. |
| 855) | 18mers_human_m_virus_1MM_1233_p.txt | Maize rayado fino virus, complete genome. |
| 856) | 18mers_human_m_virus_1MM_1234_p.txt | Oat blue dwarf virus, complete genome. |
| 857) | 18mers_human_m_virus_1MM_1235_p.txt | Poinsettia mosaic virus, complete genome. |
| 858) | 18mers_human_m_virus_1MM_1236_p.txt | Cryphonectria parasitica mitovirus 1-NB631, complete |
| | | genome. |
| 859) | 18mers_human_m_virus_1MM_1237_p.txt | Ophiostoma mitovirus 3a, complete genome. |
| 860) | 18mers_human_m_virus_1MM_1238_p.txt | Ophiostoma novo-ulmi mitovirus 4-Ld, complete genome. |
| 861) | 18mers_human_m_virus_1MM_1239_p.txt | Ophiostoma novo-ulmi mitovirus 5-Ld, complete genome. |
| 862) | 18mers_human_m_virus_1MM_1240_p.txt | Ophiostoma novo-ulmi mitovirus 6-Ld, complete genome. |
| 863) | 18mers_human_m_virus_1MM_1241_p.txt | Saccharomyces cerevisiae narnavirus 20S RNA |
| | | &lt; W&gt;, complete genome. |
| 864) | 18mers_human_m_virus_1MM_1242_p.txt | Saccharomyces cerevisiae narnavirus 23S RNA |
| | | &lt; T&gt;, complete genome. |
| 865) | 18mers_human_m_virus_1MM_1243_p.txt | Avian infectious bronchitis virus, complete genome. |
| 866) | 18mers_human_m_virus_1MM_1244_p.txt | Bovine coronavirus, complete genome. |
| 867) | 18mers_human_m_virus_1MM_1245_p.txt | Equine arteritis virus, complete genome. |
| 868) | 18mers_human_m_virus_1MM_1246_p.txt | Human coronavirus 229E, complete genome. |
| 869) | 18mers_human_m_virus_1MM_1247_p.txt | Lactate dehydrogenase-elevating virus, complete genome. |
| 870) | 18mers_human_m_virus_1MM_1248_p.txt | Murine hepatitis virus, complete genome. |
| 871) | 18mers_human_m_virus_1MM_1249_p.txt | Porcine epidemic diarrhea virus, complete genome. |
| 872) | 18mers_human_m_virus_1MM_1250_p.txt | Porcine reproductive and respiratory syndrome virus, |
| | | complete genome. |
| 873) | 18mers_human_m_virus_1MM_1251_p.txt | SARS coronavirus, complete genome. |
| 874) | 18mers_human_m_virus_1MM_1252_p.txt | Simian hemorrhagic fever virus, complete genome. |
| 875) | 18mers_human_m_virus_1MM_1253_p.txt | Transmissible gastroenteritis virus, complete genome. |
| 876) | 18mers_human_m_virus_1MM_1254_p.txt | Black beetle virus RNA 1, complete sequence. |
| 877) | 18mers_human_m_virus_1MM_1256_p.txt | Boolarra virus, complete genome. |
| 878) | 18mers_human_m_virus_1MM_1258_p.txt | Epinephelus tauvina nervous necrosis virus RNA 1, |
| | | complete sequence. |
| 879) | 18mers_human_m_virus_1MM_1260_p.txt | Flock house virus, complete genome. |
| 880) | 18mers_human_m_virus_1MM_1262_p.txt | Nodamura virus RNA 1, complete sequence. |
| 881) | 18mers_human_m_virus_1MM_1264_p.txt | Pariacoto virus RNA1, complete sequence. |
| 882) | 18mers_human_m_virus_1MM_1266_p.txt | Striped Jack nervous necrosis virus RNA1, complete |
| | | sequence. |
| 883) | 18mers_human_m_virus_1MM_1268_p.txt | Indian peanut clump virus RNA 1, complete sequence. |
| 884) | 18mers_human_m_virus_1MM_1270_p.txt | Peanut clump virus RNA 1, complete sequence. |
| 885) | 18mers_human_m_virus_1MM_1272_p.txt | Aichi virus, complete genome. |
| 886) | 18mers_human_m_virus_1MM_1273_p.txt | Avian encephalomyelitis virus, complete genome. |
| 887) | 18mers_human_m_virus_1MM_1274_p.txt | Bovine enterovirus, complete genome. |
| 888) | 18mers_human_m_virus_1MM_1275_p.txt | Bovine kobuvirus, complete genome. |
| 889) | 18mers_human_m_virus_1MM_1276_p.txt | Encephalomyocarditis virus, complete genome. |
| 890) | 18mers_human_m_virus_1MM_1277_p.txt | Enterovirus Yanbian 96-83csf, complete genome. |
| 891) | 18mers_human_m_virus_1MM_1278_p.txt | Equine rhinitis A virus, complete genome. |
| 892) | 18mers_human_m_virus_1MM_1279_p.txt | Equine rhinitis B virus, complete genome. |
| 893) | 18mers_human_m_virus_1MM_1280_p.txt | Equine rhinovirus 3, complete genome. |
| 894) | 18mers_human_m_virus_1MM_1281_p.txt | Foot-and-mouth disease virus C, complete genome. |
| 895) | 18mers_human_m_virus_1MM_1282_p.txt | Foot-and-mouth disease virus O, complete genome. |
| 896) | 18mers_human_m_virus_1MM_1283_p.txt | Foot-and-mouth disease virus SAT 2, complete genome. |
| 897) | 18mers_human_m_virus_1MM_1284_p.txt | Hepatitis A virus, complete genome. |
| 898) | 18mers_human_m_virus_1MM_1285_p.txt | Human echovirus I, complete genome. |
| 899) | 18mers_human_m_virus_1MM_1286_p.txt | Human enterovirus A, complete genome. |
| 900) | 18mers_human_m_virus_1MM_1287_p.txt | Human enterovirus B, complete genome. |
| 901) | 18mers_human_m_virus_1MM_1288_p.txt | Human enterovirus C, complete genome. |
| 902) | 18mers_human_m_virus_1MM_1289_p.txt | Human enterovirus D, complete genome. |
| 903) | 18mers_human_m_virus_1MM_1290_p.txt | Human enterovirus E, complete genome. |
| 904) | 18mers_human_m_virus_1MM_1291_p.txt | Human parechovirus 2, complete genome. |
| 905) | 18mers_human_m_virus_1MM_1292_p.txt | Human rhinovirus 89, complete genome. |
| 906) | 18mers_human_m_virus_1MM_1293_p.txt | Human rhinovirus B, complete genome. |
| 907) | 18mers_human_m_virus_1MM_1294_p.txt | Ljungan virus, complete genome. |
| 908) | 18mers_human_m_virus_1MM_1295_p.txt | Poliovirus, complete genome. |
| 909) | 18mers_human_m_virus_1MM_1296_p.txt | Porcine enterovirus A, complete genome. |
| 910) | 18mers_human_m_virus_1MM_1297_p.txt | Porcine enterovirus B, complete genome. |
| 911) | 18mers_human_m_virus_1MM_1298_p.txt | Porcine teschovirus I, complete genome. |
| 912) | 18mers_human_m_virus_1MM_1299_p.txt | Simian picornavirus 1, complete genome. |
| 913) | 18mers_human_m_virus_1MM_1300_p.txt | Theilovirus, complete genome. |
| 914) | 18mers_human_m_virus_1MM_1301_p.txt | Bamboo mosaic virus, complete genome. |
| 915) | 18mers_human_m_virus_1MM_1302_p.txt | Cassava common mosaic virus, complete genome. |
| 916) | 18mers_human_m_virus_1MM_1303_p.txt | Clover yellow mosaic virus, complete genome. |
| 917) | 18mers_human_m_virus_1MM_1304_p.txt | Cymbidium mosaic virus, complete genome. |
| 918) | 18mers_human_m_virus_1MM_1305_p.txt | Foxtail mosaic virus, complete genome. |
| 919) | 18mers_human_m_virus_1MM_1306_p.txt | Narcissus mosaic virus, complete genome. |
| 920) | 18mers_human_m_virus_1MM_1307_p.txt | Papaya mosaic virus, complete genome. |
| 921) | 18mers_human_m_virus_1MM_1308_p.txt | Pepino mosaic virus, complete genome. |
| 922) | 18mers_human_m_virus_1MM_1309_p.txt | Plantago asiatica mosaic virus, complete genome. |
| 923) | 18mers_human_m_virus_1MM_1310_p.txt | Potato aucuba mosaic virus, complete genome. |
| 924) | 18mers_human_m_virus_1MM_1311_p.txt | Potato virus X, complete genome. |
| 925) | 18mers_human_m_virus_1MM_1312_p.txt | Scallion virus X, complete genome. |
| 926) | 18mers_human_m_virus_1MM_1313_p.txt | Strawberry mild yellow edge virus, complete genome. |
| 927) | 18mers_human_m_virus_1MM_1314_p.txt | Tulip virus X, complete genome. |
| 928) | 18mers_human_m_virus_1MM_1315_p.txt | White clover mosaic virus, complete genome. |
| 929) | 18mers_human_m_virus_1MM_1316_p.txt | Barley mild mosaic virus RNA 1, complete sequence. |
| 930) | 18mers_human_m_virus_1MM_1318_p.txt | Barley yellow mosaic virus RNA 1, complete sequence. |
| 931) | 18mers_human_m_virus_1MM_1320_p.txt | Bean common mosaic necrosis virus, complete genome. |
| 932) | 18mers_human_m_virus_1MM_1321_p.txt | Bean common mosaic virus, complete genome. |
| 933) | 18mers_human_m_virus_1MM_1322_p.txt | Bean yellow mosaic virus, complete genome. |
| 934) | 18mers_human_m_virus_1MM_1323_p.txt | Brome streak mosaic virus, complete genome. |
| 935) | 18mers_human_m_virus_1MM_1324_p.txt | Clover yellow vein virus, complete genome. |
| 936) | 18mers_human_m_virus_1MM_1325_p.txt | Cocksfoot streak virus, complete genome. |
| 937) | 18mers_human_m_virus_1MM_1326_p.txt | Cowpea aphid-borne mosaic virus, complete genome. |
| 938) | 18mers_human_m_virus_1MM_1327_p.txt | Dasheen mosaic virus, complete genome. |
| 939) | 18mers_human_m_virus_1MM_1328_p.txt | Japanese yam mosaic virus, complete genome. |
| 940) | 18mers_human_m_virus_1MM_1329_p.txt | Johnsongrass mosaic virus, complete genome. |
| 941) | 18mers_human_m_virus_1MM_1330_p.txt | Leek yellow stripe potyvirus, complete genome. |
| 942) | 18mers_human_m_virus_1MM_1331_p.txt | Lettuce mosaic virus, complete genome. |
| 943) | 18mers_human_m_virus_1MM_1332_p.txt | Maize dwarf mosaic virus, complete genome. |
| 944) | 18mers_human_m_virus_1MM_1333_p.txt | Oat mosaic virus RNA 1, complete sequence. |
| 945) | 18mers_human_m_virus_1MM_1335_p.txt | Papaya ringspot virus, complete genome. |
| 946) | 18mers_human_m_virus_1MM_1336_p.txt | Pea seed-borne mosaic virus, complete genome. |
| 947) | 18mers_human_m_virus_1MM_1337_p.txt | Peanut mottle virus, complete genome. |
| 948) | 18mers_human_m_virus_1MM_1338_p.txt | Pepper mottle virus, complete genome. |
| 949) | 18mers_human_m_virus_1MM_1339_p.txt | Peru tomato mosaic virus, complete genome. |
| 950) | 18mers_human_m_virus_1MM_1340_p.txt | Potato virus V, complete genome. |
| 951) | 18mers_human_m_virus_1MM_1341_p.txt | Potato virus Y, complete genome. |
| 952) | 18mers_human_m_virus_1MM_1342_p.txt | Ryegrass mosaic virus, complete genome. |
| 953) | 18mers_human_m_virus_1MM_1343_p.txt | Scallion mosaic virus, complete genome. |
| 954) | 18mers_human_m_virus_1MM_1344_p.txt | Sorghum mosaic virus, complete genome. |
| 955) | 18mers_human_m_virus_1MM_1345_p.txt | Soybean mosaic virus, complete genome. |
| 956) | 18mers_human_m_virus_1MM_1346_p.txt | Sugarcane mosaic virus, complete genome. |
| 957) | 18mers_human_m_virus_1MM_1347_p.txt | Sweet potato feathery mottle virus, complete genome. |
| 958) | 18mers_human_m_virus_1MM_1348_p.txt | Sweet potato mild mottle virus, complete genome. |
| 959) | 18mers_human_m_virus_1MM_1349_p.txt | Tobacco etch virus, complete genome. |
| 960) | 18mers_human_m_virus_1MM_1350_p.txt | Tobacco vein mottling virus, complete genome. |
| 961) | 18mers_human_m_virus_1MM_1351_p.txt | Turnip mosaic virus, complete genome. |
| 962) | 18mers_human_m_virus_1MM_1352_p.txt | Wheat streak mosaic virus, complete genome. |
| 963) | 18mers_human_m_virus_1MM_1353_p.txt | Wheat yellow mosaic virus RNA 1, complete sequence. |
| 964) | 18mers_human_m_virus_1MM_1355_p.txt | Wild potato mosaic virus, complete genome. |
| 965) | 18mers_human_m_virus_1MM_1356_p.txt | Yam mosaic virus, complete genome. |
| 966) | 18mers_human_m_virus_1MM_1357_p.txt | Zucchini yellow mosaic virus, complete genome. |
| 967) | 18mers_human_m_virus_1MM_1358_p.txt | Apple latent spherical virus segment 1, complete |
| | | sequence. |
| 968) | 18mers_human_m_virus_1MM_1360_p.txt | Maize chlorotic dwarf virus, complete genome. |
| 969) | 18mers_human_m_virus_1MM_1361_p.txt | Parsnip yellow fleck virus, complete genome. |
| 970) | 18mers_human_m_virus_1MM_1362_p.txt | Rice tungro spherical virus, complete genome. |
| 971) | 18mers_human_m_virus_1MM_1363_p.txt | Satsuma dwarf virus RNA1, complete sequence. |
| 972) | 18mers_human_m_virus_1MM_1365_p.txt | Strawberry mottle virus RNA2, complete sequence. |
| 973) | 18mers_human_m_virus_1MM_1367_p.txt | Cocksfoot mottle virus, complete genome. |
| 974) | 18mers_human_m_virus_1MM_1368_p.txt | Lucerne transient streak virus, complete genome. |
| 975) | 18mers_human_m_virus_1MM_1369_p.txt | Rice yellow mottle virus, complete genome. |
| 976) | 18mers_human_m_virus_1MM_1370_p.txt | Sesbania mosaic virus, complete genome. |
| 977) | 18mers_human_m_virus_1MM_1371_p.txt | Southern bean mosaic virus, complete genome. |
| 978) | 18mers_human_m_virus_1MM_1372_p.txt | Southern cowpea mosaic virus, complete genome. |
| 979) | 18mers_human_m_virus_1MM_1373_p.txt | Subterranean clover mottle virus, complete genome. |
| 980) | 18mers_human_m_virus_1MM_1374_p.txt | Turnip rosette virus, complete genome. |
| 981) | 18mers_human_m_virus_1MM_1375_p.txt | Helicoverpa armigera stunt virus RNA 1, complete |
| | | sequence. |
| 982) | 18mers_human_m_virus_1MM_1377_p.txt | Nudaurclia capensis beta virus, complete genome. |
| 983) | 18mers_human_m_virus_1MM_1378_p.txt | Crucifer tobamovirus, complete genome. |
| 984) | 18mers_human_m_virus_1MM_1379_p.txt | Cucumber fruit mottle mosaic virus, complete genome. |
| 985) | 18mers_human_m_virus_1MM_1380_p.txt | Cucumber green mottle mosaic virus, complete genome. |
| 986) | 18mers_human_m_virus_1MM_1381_p.txt | Kyuri green mottle mosaic virus, complete genome. |
| 987) | 18mers_human_m_virus_1MM_1382_p.txt | Obuda pepper virus, complete genome. |
| 988) | 18mers_human_m_virus_1MM_1383_p.txt | Odontoglossum ringspot virus, complete genome. |
| 989) | 18mers_human_m_virus_1MM_1384_p.txt | Paprika mild mottle virus, complete genome. |
| 990) | 18mers_human_m_virus_1MM_1385_p.txt | Pepper mild mottle virus, complete genome. |
| 991) | 18mers_human_m_virus_1MM_1386_p.txt | Ribgrass mosaic virus, complete genome. |
| 992) | 18mers_human_m_virus_1MM_1387_p.txt | Tobacco mild green mosaic virus, complete genome. |
| 993) | 18mers_human_m_virus_1MM_1388_p.txt | Tobacco mosaic virus, complete genome. |
| 994) | 18mers_human_m_virus_1MM_1389_p.txt | Tomato mosaic virus, complete genome. |
| 995) | 18mers_human_m_virus_1MM_1390_p.txt | Turnip vein-clearing virus, complete genome. |
| 996) | 18mers_human_m_virus_1MM_1391_p.txt | Youcai mosaic virus, complete genome. |
| 997) | 18mers_human_m_virus_1MM_1392_p.txt | Zucchini green mottle mosaic virus, complete genome. |
| 998) | 18mers_human_m_virus_1MM_1393_p.txt | Pea early browning virus RNA 1, complete sequence. |
| 999) | 18mers_human_m_virus_1MM_1395_p.txt | Pepper ringspot virus RNA 1, complete sequence. |
| 1000) | 18mers_human_m_virus_1MM_1397_p.txt | Tobacco rattle virus RNA 1. complete sequence. |
| 1001) | 18mers_human_m_virus_1MM_1399_p.txt | Aura virus, complete genome. |
| 1002) | 18mers_human_m_virus_1MM_1400_p.txt | Barmah Forest virus, complete genome. |
| 1003) | 18mers_human_m_virus_1MM_1401_p.txt | Chikungunya virus, complete genome. |
| 1004) | 18mers_human_m_virus_1MM_1402_p.txt | Eastern equine encephalitis virus, complete |
| | | genome. |
| 1005) | 18mers_human_m_virus_1MM_1403_p.txt | Igbo Ora virus, complete genome. |
| 1006) | 18mers_human_m_virus_1MM_1404_p.txt | Mayaro virus, complete genome. |
| 1007) | 18mers_human_m_virus_1MM_1405_p.txt | O'nyong-nyong virus, complete genome. |
| 1008) | 18mers_human_m_virus_1MM_1406_p.txt | Ross River virus, complete genome. |
| 1009) | 18mers_human_m_virus_1MM_1407_p.txt | Rubella virus, complete genome. |
| 1010) | 18mers_human_m_virus_1MM_1408_p.txt | Salmon pancreas disease virus, complete |
| | | genome. |
| 1011) | 18mers_human_m_virus_1MM_1409_p.txt | Semliki forest virus, complete genome. |
| 1012) | 18mers_human_m_virus_1MM_1410_p.txt | Sindbis virus, complete genome. |
| 1013) | 18mers_human_m_virus_1MM_1411_p.txt | Sleeping disease virus, complete genome. |
| 1014) | 18mers_human_m_virus_1MM_1412_p.txt | Venezuelan equine encephalitis virus, complete |
| | | genome. |
| 1015) | 18mers_human_m_virus_1MM_1413_p.txt | Western equine encephalomyelitis virus, |
| | | complete genome. |
| 1016) | 18mers_human_m_virus_1MM_1414_p.txt | Artichoke mottled crinkle virus, complete |
| | | genome. |
| 1017) | 18mers_human_m_virus_1MM_1415_p.txt | Beet black scorch virus, complete genome. |
| 1018) | 18mers_human_m_virus_1MM_1416_p.txt | Cardamine chlorotic fleck virus, complete |
| | | genome. |
| 1019) | 18mers_human_m_virus_1MM_1417_p.txt | Carnation Italian ringspot virus, complete |
| | | genome. |
| 1020) | 18mers_human_m_virus_1MM_1418_p.txt | Carnation mottle virus, complete genome. |
| 1021) | 18mers_human_m_virus_1MM_1419_p.txt | Carnation ringspot virus RNA 1, complete |
| | | sequence. |
| 1022) | 18mers_human_m_virus_1MM_1421_p.txt | Cowpea mottle virus, complete genome. |
| 1023) | 18mers_human_m_virus_1MM_1422_p.txt | Cucumber Bulgarian virus, complete genome. |
| 1024) | 18mers_human_m_virus_1MM_1423_p.txt | Cucumber necrosis virus, complete genome. |
| 1025) | 18mers_human_m_virus_1MM_1424_p.txt | Cymbidium ringspot virus, complete genome. |
| 1026) | 18mers_human_m_virus_1MM_1425_p.txt | Galinsoga mosaic virus, complete genome. |
| 1027) | 18mers_human_m_virus_1MM_1426_p.txt | Hibiscus chlorotic ringspot virus, complete |
| | | genome. |
| 1028) | 18mers_human_m_virus_1MM_1427_p.txt | Japanese iris necrotic ring virus, complete |
| | | genome. |
| 1029) | 18mers_human_m_virus_1MM_1428_p.txt | Leek white stripe virus, complete genome. |
| 1030) | 18mers_human_m_virus_1MM_1429_p.txt | Maize chlorotic mottle virus, complete genome. |
| 1031) | 18mers_human_m_virus_1MM_1430_p.txt | Melon necrotic spot virus, genome. |
| 1032) | 18mers_human_m_virus_1MM_1431_p.txt | Oat chlorotic stunt virus, complete genome. |
| 1033) | 18mers_human_m_virus_1MM_1432_p.txt | Olive latent virus 1, complete genome. |
| 1034) | 18mers_human_m_virus_1MM_1433_p.txt | Panicum mosaic virus, complete genome. |
| 1035) | 18mers_human_m_virus_1MM_1434_p.txt | Pear latent virus, complete genome. |
| 1036) | 18mers_human_m_virus_1MM_1435_p.txt | Pothos latent virus, complete genome. |
| 1037) | 18mers_human_m_virus_1MM_1436_p.txt | Red clover necrotic mosaic virus RNA 1, |
| | | complete sequence. |
| 1038) | 18mers_human_m_virus_1MM_1438_p.txt | Saguaro cactus virus, complete genome. |
| 1039) | 18mers_human_m_virus_1MM_1439_p.txt | Sweet clover necrotic mosaic virus RNA 1, |
| | | complete sequence. |
| 1040) | 18mers_human_m_virus_1MM_1441_p.txt | Tobacco necrosis virus A, complete genome. |
| 1041) | 18mers_human_m_virus_1MM_1442_p.txt | Tobacco necrosis virus D, complete genome. |
| 1042) | 18mers_human_m_virus_1MM_1443_p.txt | Tomato bushy stunt virus, complete genome. |
| 1043) | 18mers_human_m_virus_1MM_1444_p.txt | Turnip crinkle virus, complete genome. |
| 1044) | 18mers_human_m_virus_1MM_1445_p.txt | Apple chlorotic leaf spot virus, complete |
| | | genome. |
| 1045) | 18mers_human_m_virus_1MM_1446_p.txt | Cherry mottle leaf virus, complete genome. |
| 1046) | 18mers_human_m_virus_1MM_1447_p.txt | Chayote mosaic tymovirus, complete genome. |
| 1047) | 18mers_human_m_virus_1MM_1448_p.txt | Eggplant mosaic virus, complete genome. |
| 1048) | 18mers_human_m_virus_1MM_1449_p.txt | Erysimum latent virus, complete genome. |
| 1049) | 18mers_human_m_virus_1MM_1450_p.txt | Kennedya yellow mosaic virus, complete |
| | | genome. |
| 1050) | 18mers_human_m_virus_1MM_1451_p.txt | Ononis yellow mosaic virus, complete genome. |
| 1051) | 18mers_human_m_virus_1MM_1452_p.txt | Physalis mottle virus, complete genome. |
| 1052) | 18mers_human_m_virus_1MM_1453_p.txt | Turnip yellow mosaic virus, complete genome. |
| 1053) | 18mers_human_m_virus_1MM_1454_p.txt | Carrot mottle mimic virus, complete genome. |
| 1054) | 18mers_human_m_virus_1MM_1455_p.txt | Groundnut rosette virus, complete genome. |
| 1055) | 18mers_human_m_virus_1MM_1456_p.txt | Pea enation mosaic virus-2, complete genome. |
| 1056) | 18mers_human_m_virus_1MM_1457_p.txt | Tobacco bushy top virus, complete genome. |
| 1057) | 18mers_human_m_virus_1MM_1458_p.txt | Grapevine virus A, complete genome. |
| 1058) | 18mers_human_m_virus_1MM_1459_p.txt | Grapevine virus B. complete genome. |
| 1059) | 18mers_human_m_virus_1MM_1460_p.txt | Acyrthosiphon pisum virus, complete genome. |
| 1060) | 18mers_human_m_virus_1MM_1461_p.txt | Beet western yellows ST9 associated virus, |
| | | complete genome. |
| 1061) | 18mers_human_m_virus_1MM_1462_p.txt | Botrytis virus F, complete genome. |
| 1062) | 18mers_human_m_virus_1MM_1463_p.txt | Citrus leaf blotch virus, complete genome. |
| 1063) | 18mers_human_m_virus_1MM_1464_p.txt | Euprosterna elaeasa virus, complete genome. |
| 1064) | 18mers_human_m_virus_1MM_1465_p.txt | Grapevine fleck virus, complete genome. |
| 1065) | 18mers_human_m_virus_1MM_1466_p.txt | Indian citrus ringspot virus, complete genome. |
| 1066) | 18mers_human_m_virus_1MM_1467_p.txt | Oyster mushroom spherical virus, complete |
| | | genome. |
| 1067) | 18mers_human_m_virus_1MM_1468_p.txt | Sugarcane striate mosaic associated virus, |
| | | complete genome. |
| 1068) | 18mers_human_m_virus_1MM_1469_p.txt | Bacteriophage IN93, complete genome. |
| 1069) | 18mers_human_m_virus_1MM_1470_p.txt | Bacteriophage L5, complete genome. |
| 1070) | 18mers_human_m_virus_1MM_1471_p.txt | Bacteriophage Mx8, complete genome. |
| 1071) | 18mers_human_m_virus_1MM_1472_p.txt | Mycobacteriophage Barnyard virion, complete |
| | | genome. |
| 1072) | 18mers_human_m_virus_1MM_1473_p.txt | Mycobacteriophage Bxzl virion, complete |
| | | genome. |
| 1073) | 18mers_human_m_virus_1MM_1474_p.txt | Mycobacteriophage Bxz2 virion, complete |
| | | genome. |
| 1074) | 18mers_human_m_virus_1MM_1475_p.txt | Mycobacteriophage Che8 virion, complete |
| | | genome. |
| 1075) | 18mers_human_m_virus_1MM_1476_p.txt | Mycobacteriophage Che9c virion, complete |
| | | genome. |
| 1076) | 18mers_human_m_virus_1MM_1477_p.txt | Mycobacteriophage Che9d virion, complete |
| | | genome. |
| 1077) | 18mers_human_m_virus_1MM_1478_p.txt | Mycobacteriophage CJW1 virion, complete |
| | | genome. |
| 1078) | 18mers_human_m_virus_1MM_1479_p.txt | Mycobacteriophage Corndog virion, complete |
| | | genome. |
| 1079) | 18mers_human_m_virus_1MM_1480_p.txt | Mycobacteriophage Omega virion, complete |
| | | genome. |
| 1080) | 18mers_human_m_virus_1MM_1481_p.txt | Mycobacteriophage Rosebush virion, complete |
| | | genome. |
| 1081) | 18mers_human_m_virus_1MM_1482_p.txt | Sinorhizobium meliloti phage PBC5, complete |
| | | genome. |
| 1082) | 18mers_human_m_virus_1MM_1483_p.txt | Staphylococcus aureus phage phi 11 provirus, |
| | | complete genome. |
| 1083) | 18mers_human_m_virus_1MM_1484_p.txt | Staphylococcus aureus phage phi 12 provirus. |
| | | complete genome. |
| 1084) | 18mers_human_m_virus_1MM_1485_p.txt | Staphylococcus aureus phage phi 13 provirus, |
| | | complete genome. |
| 1085) | 18mers_human_m_virus_1MM_1486_p.txt | Staphylococcus phage phiN315 provirus, |
| | | complete genome. |
| 1086) | 18mers_human_m_virus_1MM_1487_p.txt | Streptococcus pyogenes phage 315.1 provirus, |
| | | complete genome. |
| 1087) | 18mers_human_m_virus_1MM_1488_p.txt | Streptococcus pyogenes phage 315.2 provirus, |
| | | complete genome. |
| 1088) | 18mers_human_m_virus_1MM_1489_p.txt | Streptococcus pyogenes phage 315.3 provirus, |
| | | complete genome. |
| 1089) | 18mers_human_m_virus_1MM_1490_p.txt | Streptococcus pyogenes phage 315.4 provirus, |
| | | complete genome. |
| 1090) | 18mers_human_m_virus_1MM_1491_p.txt | Streptococcus pyogenes phage 315.5 provirus, |
| | | complete genome. |
| 1091) | 18mers_human_m_virus_1MM_1492_p.txt | Streptococcus pyogenes phage 315.6 provirus, |
| | | complete genome. |
| 1092) | 18mers_human_m_virus_1MM_1493_p.txt | Carrot red leaf luteovirus associated RNA, |
| | | complete genome. |
| 1093) | 18mers_human_m_virus_1MM_1494_p.txt | Planaria asexual strain-specific virus-like |
| | | element type 1 large DNA |
| 1094) | 18mers_human_m_virus_1MM_1496_p.txt | Saccharomyces cerevisiae killer virus M1, |
| | | complete genome. |
| 1095) | 18mers_human_m_virus_1MM_1497_p.txt | Sclerophthora macrospora virus B, complete |
| | | genome. |

3) SEQ ID NO 6: Virus 17 mers 2MM - It contains 306 files, containing sequences
of length 17, which are present in the viral genome, but are not present in the human
genome. All sequences produced from these sequences with two mismatches are also
not present in the human genome. The list below contains the file name and the
corresponding virus name.

| | File Name | Species Taxonomy Name |
|---|---|---|
| 1) | 17mers_human_m_virus_2MM_15_p.txt | Petunia vein clearing virus, complete |
| | | genome. |
| 2) | 17mers_human_m_virus_2MM_41_p.txt | Feline foamy virus, complete genome. |
| 3) | 17mers_human_m_virus_2MM_67_p.txt | Simian immunodeficiency virus, complete |
| | | genome. |
| 4) | 17mers_human_m_virus_2MM_106_p.txt | Satellite tobacco mosaic virus, complete |
| | | genome. |
| 5) | 17mers_human_m_virus_2MM_161_p.txt | Bacteriophage A118 virion, complete |
| | | genome. |
| 6) | 17mers_human_m_virus_2MM_177_p.txt | Bacteriophage P4, complete genome. |
| 7) | 17mers_human_m_virus_2MM_188_p.txt | Bacteriophage sk1, complete genome. |
| 8) | 17mers_human_m_virus_2MM_190_p.txt | Bacteriophage SPP1, complete genome. |
| 9) | 17mers_human_m_virus_2MM_202_p.txt | Enterobacteria phage T7. |
| 10) | 17mers_human_m_virus_2MM_206_p.txt | Lactobacillus casei bacteriophage A2 |
| | | virion, complete genome. |
| 11) | 17mers_human_m_virus_2MM_211_p.txt | Listeria phage 2389 virion, complete |
| | | genome. |
| 12) | 17mers_human_m_virus_2MM_213_p.txt | Methanothermobacter wolfeii prophage |
| | | psiM100, complete genome. |
| 13) | 17mers_human_m_virus_2MM_221_p.txt | Pseudomonas phage gh-1, complete |
| | | genome. |
| 14) | 17mers_human_m_virus_2MM_227_p.txt | Staphylococcus aureus bacteriophage PVL |
| | | provirus, complete genome. |
| 15) | 17mers_human_m_virus_2MM_235_p.txt | Streptococcus thermophilus bacteriophage |
| | | Sfi11, complete genome. |
| 16) | 17mers_human_m_virus_2MM_240_p.txt | Temperate phage PhiN1H1.1, complete |
| | | genome. |
| 17) | 17mers_human_m_virus_2MM_244_p.txt | Yersinia pestis phage phiA1122, complete |
| | | genome. |
| 18) | 17mers_human_m_virus_2MM_246_p.txt | Sulfolobus virus 1, complete genome. |
| 19) | 17mers_human_m_virus_2MM_247_p.txt | Alcelaphine herpesvirus 1, complete |
| | | genome. |
| 20) | 17mers_human_m_virus_2MM_277_p.txt | Rana tigrina ranavirus, complete genome. |
| 21) | 17mers_human_m_virus_2MM_278_p.txt | Sulfolobus islandicus filamentous virus, |
| | | genome. |
| 22) | 17mers_human_m_virus_2MM_283_p.txt | Bovine papillomavirus type 4, complete |
| | | genome. |
| 23) | 17mers_human_m_virus_2MM_293_p.txt | Fringilla coelebs papillomavirus, complete |
| | | genome. |
| 24) | 17mers_human_m_virus_2MM_294_p.txt | Hamster papovavirus, complete genome. |
| 25) | 17mers_human_m_virus_2MM_297_p.txt | Human papillomavirus type 10, complete |
| | | genome. |
| 26) | 17mers_human_m_virus_2MM_307_p.txt | Human papillomavirus type 1a, complete |
| | | genome. |
| 27) | 17mers_human_m_virus_2MM_316_p.txt | Human papillomavirus type 28, complete |
| | | genome. |
| 28) | 17mers_human_m_virus_2MM_317_p.txt | Human papillomavirus type 29, complete |
| | | genome. |
| 29) | 17mers_human_m_virus_2MM_331_p.txt | Human papillomavirus type 40, complete |
| | | genome. |
| 30) | 17mers_human_m_virus_2MM_347_p.txt | Human papillomavirus type 57, complete |
| | | genome. |
| 31) | 17mers_human_m_virus_2MM_352_p.txt | Human papillomavirus type 60, complete |
| | | genome. |
| 32) | 17mers_human_m_virus_2MM_355_p.txt | Human papillomavirus type 65, complete |
| | | genome. |
| 33) | 17mers_human_m_virus_2MM_361_p.txt | Human papillomavirus type 7, complete |
| | | genome. |
| 34) | 17mers_human_m_virus_2MM_370_p.txt | Human papillomavirus type 87, complete |
| | | genome. |
| 35) | 17mers_human_m_virus_2MM_374_p.txt | Human papillomavirus type 91, complete |
| | | genome. |
| 36) | 17mers_human_m_virus_2MM_391_p.txt | Budgerigar fledgling polyomavirus, |
| | | complete genome. |
| 37) | 17mers_human_m_virus_2MM_437_p.txt | Cryphonectria hypovirus 1, complete |
| | | genome. |
| 38) | 17mers_human_m_virus_2MM_446_p.txt | Discula destructiva virus 2 segment 1, |
| | | complete sequence. |
| 39) | 17mers_human_m_virus_2MM_535_p.txt | Mammalian orthoreovirus 1 segment L1, |
| | | complete sequence. |
| 40) | 17mers_human_m_virus_2MM_555_p.txt | Mammalian orthoreovirus 3 segment L1, |
| | | complete sequence. |
| 41) | 17mers_human_m_virus_2MM_585_p.txt | Rice dwarf virus segment 1, complete |
| | | sequence. |
| 42) | 17mers_human_m_virus_2MM_618_p.txt | Eimeria brunetti RNA virus 1, complete |
| | | genome. |
| 43) | 17mers_human_m_virus_2MM_620_p.txt | Gremmeniella abietina RNA virus L1, |
| | | complete genome. |
| 44) | 17mers_human_m_virus_2MM_626_p.txt | Saccharomyces cerevisiae virus L-BC (La), |
| | | complete genome. |
| 45) | 17mers_human_m_virus_2MM_629_p.txt | Trichomonas vaginalis virus, complete |
| | | genome. |
| 46) | 17mers_human_m_virus_2MM_638_p.txt | Chicken anemia virus, complete genome. |
| 47) | 17mers_human_m_virus_2MM_644_p.txt | Abutilon mosaic virus A component DNA, |
| | | complete sequence. |
| 48) | 17mers_human_m_virus_2MM_679_p.txt | Cotton leaf curl Rajasthan virus segment |
| | | A, complete sequence. |
| 49) | 17mers_human_m_virus_2MM_686_p.txt | East African cassava mosaic Cameroon |
| | | virus DNA A, complete sequence. |
| 50) | 17mers_human_m_virus_2MM_704_p.txt | Macroptilium yellow mosaic virus, |
| | | complete genome. |
| 51) | 17mers_human_m_virus_2MM_729_p.txt | Sida golden mosaic Costa Rica virus DNA |
| | | A, complete sequence. |
| 52) | 17mers_human_m_virus_2MM_732_p.txt | Sida golden mosaic Honduras virus DNA |
| | | A, complete sequence. |
| 53) | 17mers_human_m_virus_2MM_739_p.txt | Sida yellow vein virus DNA A, complete |
| | | sequence. |
| 54) | 17mers_human_m_virus_2MM_802_p.txt | Watermelon chlorotic stunt virus DNA A, |
| | | complete sequence. |
| 55) | 17mers_human_m_virus_2MM_809_p.txt | Enterobacteria phage 12-2. |
| 56) | 17mers_human_m_virus_2MM_812_p.txt | Propionibacterium phage phiB5, complete |
| | | genome. |
| 57) | 17mers_human_m_virus_2MM_813_p.txt | Pseudomonas phage Pf1, complete |
| | | genome. |
| 58) | 17mers_human_m_virus_2MM_818_p.txt | Vibrio cholerae filamentous bacteriophage |
| | | fs-2, complete genome. |
| 59) | 17mers_human_m_virus_2MM_823_p.txt | Chlamydia phage 2 virion, complete |
| | | genome. |
| 60) | 17mers_human_m_virus_2MM_828_p.txt | Coliphage phiX174, complete genome. |
| 61) | 17mers_human_m_virus_2MM_830_p.txt | Enterobacteria phage S13, complete |
| | | genome. |
| 62) | 17mers_human_m_virus_2MM_831_p.txt | Phage phiMH2K virion, complete genome. |
| 63) | 17mers_human_m_virus_2MM_834_p.txt | Banana bunchy top virus DNA 1, complete |
| | | sequence. |
| 64) | 17mers_human_m_virus_2MM_885_p.txt | Diatraea saccharalis densovirus, complete |
| | | genome. |
| 65) | 17mers_human_m_virus_2MM_948_p.txt | Newcastle disease virus, complete genome. |
| 66) | 17mers_human_m_virus_2MM_953_p.txt | Reston Ebola virus, complete genome. |
| 67) | 17mers_human_m_virus_2MM_1033_p.txt | Beet soil-borne mosaic virus RNA1, |
| | | complete sequence. |
| 68) | 17mers_human_m_virus_2MM_1049_p.txt | Brome mosaic virus RNA 1, complete |
| | | sequence. |
| 69) | 17mers_human_m_virus_2MM_1052_p.txt | Citrus leaf rugose virus RNA 1, complete |
| | | sequence. |
| 70) | 17mers_human_m_virus_2MM_1055_p.txt | Cowpea chlorotic mottle virus RNA 1, |
| | | complete sequence. |
| 71) | 17mers_human_m_virus_2MM_1091_p.txt | Calicivirus strain NB, complete genome. |
| 72) | 17mers_human_m_virus_2MM_1102_p.txt | Aconitum latent virus, complete genome. |
| 73) | 17mers_human_m_virus_2MM_1106_p.txt | Potato virus M, complete genome. |
| 74) | 17mers_human_m_virus_2MM_1109_p.txt | Cucumber yellows virus RNA 1, complete |
| | | sequence. |
| 75) | 17mers_human_m_virus_2MM_1146_p.txt | Acute bee paralysis virus, complete |
| | | genome. |
| 76) | 17mers_human_m_virus_2MM_1148_p.txt | Black queen cell virus, complete genome. |
| 77) | 17mers_human_m_virus_2MM_1160_p.txt | Deer tick virus, genome. |
| 78) | 17mers_human_m_virus_2MM_1163_p.txt | Hepatitis G virus, complete genome. |
| 79) | 17mers_human_m_virus_2MM_1164_p.txt | Hepatitis GB virus A, complete genome. |
| 80) | 17mers_human_m_virus_2MM_1165_p.txt | Hepatitis GB virus B, complete genome. |
| 81) | 17mers_human_m_virus_2MM_1168_p.txt | Langat virus, complete genome. |
| 82) | 17mers_human_m_virus_2MM_1175_p.txt | Pestivirus type 2, complete genome. |
| 83) | 17mers_human_m_virus_2MM_1208_p.txt | Bacteriophage M11, complete genome. |
| 84) | 17mers_human_m_virus_2MM_1210_p.txt | Enterobacteria phage fr, complete genome. |
| 85) | 17mers_human_m_virus_2MM_1216_p.txt | Pseudomonas phage PP7, complete |
| | | genome. |
| 86) | 17mers_human_m_virus_2MM_1223_p.txt | Beet mild yellowing virus, complete |
| | | genome. |
| 87) | 17mers_human_m_virus_2MM_1230_p.txt | Soybean dwarf virus, complete genome. |
| 88) | 17mers_human_m_virus_2MM_1231_p.txt | Sugarcane yellow leaf virus, complete |
| | | genome. |
| 89) | 17mers_human_m_virus_2MM_1242_p.txt | Saccharomyces cerevisiae narnavirus 23S |
| | | RNA &lt; T&gt;, complete genome. |
| 90) | 17mers_human_m_virus_2MM_1251_p.txt | SARS coronavirus, complete genome. |
| 91) | 17mers_human_m_virus_2MM_1252_p.txt | Simian hemorrhagic fever virus, complete |
| | | genome. |
| 92) | 17mers_human_m_virus_2MM_1256_p.txt | Boolarra virus, complete genome. |
| 93) | 17mers_human_m_virus_2MM_1264_p.txt | Pariacoto virus RNA1, complete sequence. |
| 94) | 17mers_human_m_virus_2MM_1287_p.txt | Human enterovirus B, complete genome. |
| 95) | 17mers_human_m_virus_2MM_1300_p.txt | Theilovirus, complete genome. |
| 96) | 17mers_human_m_virus_2MM_1345_p.txt | Soybean mosaic virus, complete genome. |
| 97) | 17mers_human_m_virus_2MM_1355_p.txt | Wild potato mosaic virus, complete |
| | | genome. |
| 98) | 17mers_human_m_virus_2MM_1362_p.txt | Rice tungro spherical virus, complete |
| | | genome. |
| 99) | 17mers_human_m_virus_2MM_1368_p.txt | Lucerne transient streak virus, complete |
| | | genome. |
| 100) | 17mers_human_m_virus_2MM_1375_p.txt | Helicoverpa armigera stunt virus |
| | | RNA 1, complete sequence. |
| 101) | 17mers_human_m_virus_2MM_1378_p.txt | Crucifer tobamovirus, complete |
| | | genome. |
| 102) | 17mers_human_m_virus_2MM_1381_p.txt | Kyuri green mottle mosaic virus, |
| | | complete genome. |
| 103) | 17mers_human_m_virus_2MM_1387_p.txt | Tobacco mild green mosaic virus, |
| | | complete genome. |
| 104) | 17mers_human_m_virus_2MM_1390_p.txt | Turnip vein-clearing virus, |
| | | complete genome. |
| 105) | 17mers_human_m_virus_2MM_1392_p.txt | Zucchini green mottle mosaic |
| | | virus, complete genome. |
| 106) | 17mers_human_m_virus_2MM_1399_p.txt | Aura virus, complete genome. |
| 107) | 17mers_human_m_virus_2MM_1401_p.txt | Chikungunya virus, complete |
| | | genome. |
| 108) | 17mers_human_m_virus_2MM_1406_p.txt | Ross River virus, complete |
| | | genome. |
| 109) | 17mers_human_m_virus_2MM_1423_p.txt | Cucumber necrosis virus, |
| | | complete genome. |
| 110) | 17mers_human_m_virus_2MM_1424_p.txt | Cymbidium ringspot virus, |
| | | complete genome. |
| 111) | 17mers_human_m_virus_2MM_1438_p.txt | Saguaro cactus virus, complete |
| | | genome. |
| 112) | 17mers_human_m_virus_2MM_1457_p.txt | Tobacco bushy top virus, |
| | | complete genome. |
| 113) | 17mers_human_m_virus_2MM_1465_p.txt | Grapevine fleck virus, complete |
| | | genome. |
| 114) | 17mers_human_m_virus_2MM_1469_p.txt | Bacteriophage IN93, complete |
| | | genome. |
| 115) | 17mers_human_m_virus_2MM_1490_p.txt | Streptococcus pyogenes phage |
| | | 315.4 provirus, complete genome. |
| 116) | 17mers_human_m_virus_2MM_121_p.txt | Bovine adenovirus B, complete |
| | | genome. |
| 117) | 17mers_human_m_virus_2MM_131_p.txt | Human adenovirus D, complete |
| | | genome. |
| 118) | 17mers_human_m_virus_2MM_132_p.txt | Human adenovirus E, complete |
| | | genome. |
| 119) | 17mers_human_m_virus_2MM_157_p.txt | Acyrthosiphon pisum |
| | | bacteriophage APSE-1, complete genome. |
| 120) | 17mers_human_m_virus_2MM_166_p.txt | Bacteriophage bIL309, complete |
| | | genome. |
| 121) | 17mers_human_m_virus_2MM_172_p.txt | Bacteriophage K139, complete |
| | | genome. |
| 122) | 17mers_human_m_virus_2MM_180_p.txt | Bacteriophage phi-105, complete |
| | | genome. |
| 123) | 17mers_human_m_virus_2MM_185_p.txt | Bacteriophage phigle, complete |
| | | genome. |
| 124) | 17mers_human_m_virus_2MM_191_p.txt | Bacteriophage T3, complete |
| | | genome. |
| 125) | 17mers_human_m_virus_2MM_201_p.txt | Enterobacteria phage T4, |
| | | complete genome. |
| 126) | 17mers_human_m_virus_2MM_212_p.txt | Methanobacterium phage psiM2, |
| | | complete genome. |
| 127) | 17mers_human_m_virus_2MM_219_p.txt | Pseudomonas aeruginosa phage |
| | | PaP3, complete genome. |
| 128) | 17mers_human_m_virus_2MM_233_p.txt | Streptococcus thermophilus |
| | | bacteriophage 7201, complete genome. |
| 129) | 17mers_human_m_virus_2MM_241_p.txt | Vibrio harveyi bacteriophage |
| | | VHML, complete genome. |
| 130) | 17mers_human_m_virus_2MM_242_p.txt | Vibriophage VpV262, complete |
| | | genome. |
| 131) | 17mers_human_m_virus_2MM_245_p.txt | Alteromonas phage PM2, |
| | | complete genome. |
| 132) | 17mers_human_m_virus_2MM_265_p.txt | Human herpesvirus 7, complete |
| | | genome. |
| 133) | 17mers_human_m_virus_2MM_276_p.txt | Lymphocystis disease virus 1, |
| | | complete genome. |
| 134) | 17mers_human_m_virus_2MM_279_p.txt | Shrimp white spot syndrome |
| | | virus, complete genome. |
| 135) | 17mers_human_m_virus_2MM_318_p.txt | Human papillomavirus type 2a, |
| | | complete genome. |
| 136) | 17mers_human_m_virus_2MM_319_p.txt | Human papillomavirus type 3, |
| | | complete genome. |
| 137) | 17mers_human_m_virus_2MM_414_p.txt | Enterobacteria phage PRD1, |
| | | complete genome. |
| 138) | 17mers_human_m_virus_2MM_436_p.txt | Cryphonectria hypovirus, |
| | | complete genome. |
| 139) | 17mers_human_m_virus_2MM_455_p.txt | Rhizoctonia solani virus RNA1, |
| | | complete sequence. |
| 140) | 17mers_human_m_virus_2MM_597_p.txt | Rice ragged stunt virus segment 1, |
| | | complete sequence. |
| 141) | 17mers_human_m_virus_2MM_621_p.txt | Helminthosporium victoriae virus |
| | | 190S, complete genome. |
| 142) | 17mers_human_m_virus_2MM_627_p.txt | Sphaeropsis sapinea RNA virus 1, |
| | | complete genome. |
| 143) | 17mers_human_m_virus_2MM_632_p.txt | Ustilago maydis virus H1, |
| | | complete genome. |
| 144) | 17mers_human_m_virus_2MM_675_p.txt | Cotton leaf curl Alabad virus, |
| | | complete genome. |
| 145) | 17mers_human_m_virus_2MM_678_p.txt | Cotton leaf curl Multan virus, |
| | | complete genome. |
| 146) | 17mers_human_m_virus_2MM_697_p.txt | Indian cassava mosaic virus DNA |
| | | B, complete sequence. |
| 147) | 17mers_human_m_virus_2MM_713_p.txt | Okra yellow vein mosaic virus, |
| | | complete genome. |
| 148) | 17mers_human_m_virus_2MM_752_p.txt | Sri Lankan cassava mosaic virus |
| | | DNA A, complete sequence. |
| 149) | 17mers_human_m_virus_2MM_805_p.txt | Bacteriophage VfO3K6, complete |
| | | genome. |
| 150) | 17mers_human_m_virus_2MM_806_p.txt | Bacteriophage VfO4K68, |
| | | complete genome. |
| 151) | 17mers_human_m_virus_2MM_810_p.txt | Enterobacteria phage 1f1, |
| | | complete genome. |
| 152) | 17mers_human_m_virus_2MM_872_p.txt | Adeno-associated virus 2, |
| | | complete genome. |
| 153) | 17mers_human_m_virus_2MM_875_p.txt | Adeno-associated virus 4, |
| | | complete genome. |
| 154) | 17mers_human_m_virus_2MM_886_p.txt | Galleria mellonella densovirus, |
| | | complete genome. |
| 155) | 17mers_human_m_virus_2MM_890_p.txt | Junonia coenia densovirus, |
| | | complete genome. |
| 156) | 17mers_human_m_virus_2MM_898_p.txt | Porcine parvovirus, complete |
| | | genome. |
| 157) | 17mers_human_m_virus_2MM_1019_p.txt | Garlic virus E, complete genome. |
| 158) | 17mers_human_m_virus_2MM_1040_p.txt | American plum line pattern virus |
| | | RNA1, complete sequence. |
| 159) | 17mers_human_m_virus_2MM_1064_p.txt | Olive latent virus 2 RNA 1, |
| | | complete sequence. |
| 160) | 17mers_human_m_virus_2MM_1098_p.txt | Vesicular exanthema of swine |
| | | virus, complete genome. |
| 161) | 17mers_human_m_virus_2MM_1103_p.txt | Blueberry scorch virus, complete |
| | | genome. |
| 162) | 17mers_human_m_virus_2MM_1153_p.txt | Rhopalosiphum padi virus, |
| | | complete genome. |
| 163) | 17mers_human_m_virus_2MM_1192_p.txt | Soil-borne cereal mosaic virus |
| | | RNA1, complete sequence. |
| 164) | 17mers_human_m_virus_2MM_1213_p.txt | Enterobacteria phage MX1. |
| 165) | 17mers_human_m_virus_2MM_1241_p.txt | Saccharomyces cerevisiae |
| | | narnavirus 20S RNA &lt;W&gt;, complete genome. |
| 166) | 17mers_human_m_virus_2MM_1250_p.txt | Porcine reproductive and |
| | | respiratory syndrome virus, complete genome. |
| 167) | 17mers_human_m_virus_2MM_1270_p.txt | Peanut clump virus RNA 1, |
| | | complete sequence. |
| 168) | 17mers_human_m_virus_2MM_1275_p.txt | Bovine kobuvirus, complete |
| | | genome. |
| 169) | 17mers_human_m_virus_2MM_1283_p.txt | Foot-and-mouth disease virus |
| | | SAT 2, complete genome. |
| 170) | 17mers_human_m_virus_2MM_1331_p.txt | Lettuce mosaic virus, complete |
| | | genome. |
| 171) | 17mers_human_m_virus_2MM_1377_p.txt | Nudaurelia capensis beta virus, |
| | | complete genome. |
| 172) | 17mers_human_m_virus_2MM_1386_p.txt | Ribgrass mosaic virus, complete |
| | | genome. |
| 173) | 17mers_human_m_virus_2MM_1391_p.txt | Youcai mosaic virus, complete |
| | | genome. |
| 174) | 17mers_human_m_virus_2MM_1407_p.txt | Rubella virus, complete genome. |
| 175) | 17mers_human_m_virus_2MM_1416_p.txt | Cardamine chlorotic fleck virus, |
| | | complete genome. |
| 176) | 17mers_human_m_virus_2MM_1434_p.txt | Pear latent virus, complete |
| | | genome. |
| 177) | 17mers_human_m_virus_2MM_1467_p.txt | Oyster mushroom spherical virus, |
| | | complete genome. |
| 178) | 17mers_human_m_virus_2MM_1483_p.txt | Staphylococcus aureus phage phi |
| | | 11 provirus, complete genome. |
| 179) | 17mers_human_m_virus_2MM_82_p.txt | Bhendi yellow vein mosaic virus- |
| | | associated DNA beta, complete genome. |
| 180) | 17mers_human_m_virus_2MM_124_p.txt | Duck adenovirus 1, complete |
| | | genome. |
| 181) | 17mers_human_m_virus_2MM_133_p.txt | Human adenovirus F, complete |
| | | genome. |
| 182) | 17mers_human_m_virus_2MM_136_p.txt | Ovine adenovirus A, complete |
| | | genome. |
| 183) | 17mers_human_m_virus_2MM_186_p.txt | Bacteriophage phiYeO3-12, |
| | | complete genome. |
| 184) | 17mers_human_m_virus_2MM_198_p.txt | Enterobacteria phage HK022 |
| | | virion, complete genome. |
| 185) | 17mers_human_m_virus_2MM_203_p.txt | Haemophilus phage HP1, |
| | | complete genome. |
| 186) | 17mers_human_m_virus_2MM_226_p.txt | Shigella flexneri bacteriophage V, |
| | | complete genome. |
| 187) | 17mers_human_m_virus_2MM_251_p.txt | Callitrichine herpesvirus 3, |
| | | complete genome. |
| 188) | 17mers_human_m_virus_2MM_315_p.txt | Human papillomavirus type 27, |
| | | complete genome. |
| 189) | 17mers_human_m_virus_2MM_408_p.txt | Swinepox virus, complete |
| | | genome. |
| 190) | 17mers_human_m_virus_2MM_503_p.txt | Kadipiro virus segment 1, |
| | | complete sequence. |
| 191) | 17mers_human_m_virus_2MM_525_p.txt | Lymantria dispar cypovirus 14 |
| | | segment 1, complete sequence. |
| 192) | 17mers_human_m_virus_2MM_628_p.txt | Sphaeropsis sapinea RNA virus 2, |
| | | complete genome. |
| 193) | 17mers_human_m_virus_2MM_640_p.txt | Goose circovirus, complete |
| | | genome. |
| 194) | 17mers_human_m_virus_2MM_665_p.txt | Bhendi yellow vein mosaic virus, |
| | | complete genome. |
| 195) | 17mers_human_m_virus_2MM_681_p.txt | Cucurbit leaf curl virus DNA A, |
| | | complete sequence. |
| 196) | 17mers_human_m_virus_2MM_748_p.txt | Squash mild leaf curl virus DNA |
| | | A, complete sequence. |
| 197) | 17mers_human_m_virus_2MM_1144_p.txt | Tomato ringspot virus RNA 1, |
| | | complete sequence. |
| 198) | 17mers_human_m_virus_2MM_1147_p.txt | Aphid lethal paralysis virus, |
| | | complete genome. |
| 199) | 17mers_human_m_virus_2MM_1214_p.txt | Enterobacteria phage NL95. |
| 200) | 17mers_human_m_virus_2MM_1262_p.txt | Nodamura virus RNA1, complete |
| | | sequence. |
| 201) | 17mers_human_m_virus_2MM_1266_p.txt | Striped Jack nervous necrosis |
| | | virus RNA1, complete sequence. |
| 202) | 17mers_human_m_virus_2MM_1274_p.txt | Bovine enterovirus, complete |
| | | genome. |
| 203) | 17mers_human_m_virus_2MM_1409_p.txt | Semliki forest virus, complete |
| | | genome. |
| 204) | 17mers_human_m_virus_2MM_1456_p.txt | Pea enation mosaic virus-2, |
| | | complete genome. |
| 205) | 17mers_human_m_virus_2MM_139_p.txt | Tupaia adenovirus 1, complete |
| | | genome. |
| 206) | 17mers_human_m_virus_2MM_170_p.txt | Bacteriophage HK620, complete |
| | | genome. |
| 207) | 17mers_human_m_virus_2MM_171_p.txt | Bacteriophage HK97, complete |
| | | genome. |
| 208) | 17mers_human_m_virus_2MM_197_p.txt | Enterobacteria phage epsilon15, |
| | | complete genome. |
| 209) | 17mers_human_m_virus_2MM_199_p.txt | Enterobacteria phage Mu, |
| | | complete genome. |
| 210) | 17mers_human_m_virus_2MM_204_p.txt | Haemophilus phage HP2, |
| | | complete genome. |
| 211) | 17mers_human_m_virus_2MM_409_p.txt | Vaccinia virus, complete genome. |
| 212) | 17mers_human_m_virus_2MM_433_p.txt | Pseudomonas phage phi-6 |
| | | segment L, complete sequence. |
| 213) | 17mers_human_m_virus_2MM_469_p.txt | Bombyx mori cypovirus 1 |
| | | segment 1, complete sequence. |
| 214) | 17mers_human_m_virus_2MM_822_p.txt | Xanthomonas phage Cf1c, |
| | | complete genome. |
| 215) | 17mers_human_m_virus_2MM_1107_p.txt | Beet yellows virus, complete |
| | | genome. |
| 216) | 17mers_human_m_virus_2MM_1395_p.txt | Pepper ringspot virus RNA 1, |
| | | complete sequence. |
| 217) | 17mers_human_m_virus_2MM_125_p.txt | Fowl adenovirus A, complete |
| | | genome. |
| 218) | 17mers_human_m_virus_2MM_173_p.txt | Bacteriophage L-413C, complete |
| | | genome. |
| 219) | 17mers_human_m_virus_2MM_176_p.txt | Bacteriophage P27, complete |
| | | genome. |
| 220) | 17mers_human_m_virus_2MM_200_p.txt | Enterobacteria phage P2, |
| | | complete genome. |
| 221) | 17mers_human_m_virus_2MM_224_p.txt | Salmonella typhimurium |
| | | bacteriophage ST64T virion, complete genome. |
| 222) | 17mers_human_m_virus_2MM_1108_p.txt | Citrus tristeza virus, complete |
| | | genome. |
| 223) | 17mers_human_m_virus_2MM_1215_p.txt | Enterobacterio phage MS2, |
| | | complete genome. |
| 224) | 17mers_human_m_virus_2MM_410_p.txt | Variola virus, complete genome. |
| 225) | 17mers_human_m_virus_2MM_424_p.txt | Bacteriophage phi-12 segment L, |
| | | complete sequence. |
| 226) | 17mers_human_m_virus_2MM_427_p.txt | Bacteriophage phi-8 segment L, |
| | | complete sequence. |
| 227) | 17mers_human_m_virus_2MM_1112_p.txt | Grapevine rootstock stem lesion |
| | | associated virus, complete genome. |
| 228) | 17mers_human_m_virus_2MM_160_p.txt | Bacteriophage 933W provirus, |
| | | complete genome. |
| 229) | 17mers_human_m_virus_2MM_194_p.txt | Bacteriophage VT2-Sa provirus, |
| | | complete genome. |
| 230) | 17mers_human_m_virus_2MM_239_p.txt | Stx2 converting bacteriophage 1 |
| | | virion, complete genome. |
| 231) | 17mers_human_m_virus_2MM_274_p.txt | Infectious spleen and kidney |
| | | necrosis virus, complete genome. |
| 232) | 17mers_human_m_virus_2MM_545_p.txt | Mammalian orthoreovirus 2 |
| | | segment L1, complete sequence. |
| 233) | 17mers_human_m_virus_2MM_1471_p.txt | Bacteriophage Mx8, complete |
| | | genome. |
| 234) | 17mers_human_m_virus_2MM_404_p.txt | Monkeypox virus, complete |
| | | genome. |
| 235) | 17mers_human_m_virus_2MM_1111_p.txt | Grapevine leafroll-associated |
| | | virus 3, complete genome. |
| 236) | 17mers_human_m_virus_2MM_196_p.txt | Enterobacteria phage 186, |
| | | complete genome. |
| 237) | 17mers_human_m_virus_2MM_222_p.txt | Pseudomonas phage phiKZ, |
| | | complete genome. |
| 238) | 17mers_human_m_virus_2MM_256_p.txt | Equine herpesvirus 4, complete |
| | | genome. |
| 239) | 17mers_human_m_virus_2MM_399_p.txt | Ectromelia virus, complete |
| | | genome. |
| 240) | 17mers_human_m_virus_2MM_1474_p.txt | Mycobacteriophage Bxz2 virion, |
| | | complete genome. |
| 241) | 17mers_human_m_virus_2MM_174_p.txt | Bacteriophage lambda, complete |
| | | genome. |
| 242) | 17mers_human_m_virus_2MM_175_p.txt | Bacteriophage N15 virion, |
| | | complete genome. |
| 243) | 17mers_human_m_virus_2MM_225_p.txt | Salmonella typhimurium phage |
| | | ST64B, complete genome. |
| 244) | 17mers_human_m_virus_2MM_252_p.txt | Cercopithecine herpesvirus 7, |
| | | complete genome. |
| 245) | 17mers_human_m_virus_2MM_260_p.txt | Human herpesvirus 3, complete |
| | | genome. |
| 246) | 17mers_human_m_virus_2MM_266_p.txt | Human herpesvirus 8, genome. |
| 247) | 17mers_human_m_virus_2MM_397_p.txt | Camelpox virus, complete |
| | | genome. |
| 248) | 17mers_human_m_virus_2MM_220_p.txt | Pseudomonas phage D3, complete |
| | | genome. |
| 249) | 17mers_human_m_virus_2MM_430_p.txt | Pseudomonas bacteriophage phi- |
| | | 13 segment L, complete sequence. |
| 250) | 17mers_human_m_virus_2MM_215_p.txt | Mycobacteriophage D29, |
| | | complete genome. |
| 251) | 17mers_human_m_virus_2MM_264_p.txt | Human herpesvirus 6B, complete |
| | | genome. |
| 252) | 17mers_human_m_virus_2MM_398_p.txt | Cowpox virus, complete genome. |
| 253) | 17mers_human_m_virus_2MM_400_p.txt | Fowlpox virus, complete genome. |
| 254) | 17mers_human_m_virus_2MM_1479_p.txt | Mycobacteriophage Corndog |
| | | virion, complete genome. |
| 255) | 17mers_human_m_virus_2MM_1481_p.txt | Mycobacteriophage Rosebush |
| | | virion, complete genome. |
| 256) | 17mers_human_m_virus_2MM_141_p.txt | Adoxophyes honmai |
| | | nucleopolyhedrovirus, complete genome. |
| 257) | 17mers_human_m_virus_2MM_257_p.txt | Gallid herpesvirus 2, complete |
| | | genome. |
| 258) | 17mers_human_m_virus_2MM_1478_p.txt | Mycobacteriophage CJW1 virion, |
| | | complete genome. |
| 259) | 17mers_human_m_virus_2MM_153_p.txt | Plutella xylostella granulovirus, |
| | | complete genome. |
| 260) | 17mers_human_m_virus_2MM_217_p.txt | Mycobacterium phage L5. |
| 261) | 17mers_human_m_virus_2MM_1482_p.txt | Sinorhizobium meliloti phage |
| | | PBC5, complete genome. |
| 262) | 17mers_human_m_virus_2MM_178_p.txt | Bacteriophage phi CTX, complete |
| | | genome. |
| 263) | 17mers_human_m_virus_2MM_1475_p.txt | Mycobacteriophage Che8 virion, |
| | | complete genome. |
| 264) | 17mers_human_m_virus_2MM_263_p.txt | Human herpesvirus 6, complete |
| | | genome. |
| 265) | 17mers_human_m_virus_2MM_1477_p.txt | Mycobacteriophage Che9d virion, |
| | | complete genome. |
| 266) | 17mers_human_m_virus_2MM_249_p.txt | Bovine herpesvirus 1, complete |
| | | genome. |
| 267) | 17mers_human_m_virus_2MM_259_p.txt | Human herpesvirus 2, complete |
| | | genome. |
| 268) | 17mers_human_m_virus_2MM_268_p.txt | Meleagrid herpesvirus 1, complete |
| | | genome. |
| 269) | 17mers_human_m_virus_2MM_386_p.txt | Paramecium bursaria Chlorella |
| | | virus 1, complete genome. |
| 270) | 17mers_human_m_virus_2MM_154_p.txt | Rachiplusia ou multiple |
| | | nucleopolyhedrovirus, complete genome. |
| 271) | 17mers_human_m_virus_2MM_216_p.txt | Mycobacteriophage TM4, |
| | | complete genome. |
| 272) | 17mers_human_m_virus_2MM_254_p.txt | Equine herpesvirus 1, complete |
| | | genome. |
| 273) | 17mers_human_m_virus_2MM_214_p.txt | Mycobacteriophage Bxb1, |
| | | complete genome. |
| 274) | 17mers_human_m_virus_2MM_126_p.txt | Fowl adenovirus D, complete |
| | | genome. |
| 275) | 17mers_human_m_virus_2MM_1472_p.txt | Mycobacteriophage Barnyard |
| | | virion, complete genome. |
| 276) | 17mers_human_m_virus_2MM_1473_p.txt | Mycobacteriophage Bxz1 virion, |
| | | complete genome. |
| 277) | 17mers_human_m_virus_2MM_181_p.txt | Bacteriophage phi-BT1 virion, |
| | | complete genome. |
| 278) | 17mers_human_m_virus_2MM_182_p.txt | Bacteriophage phi-C31, complete |
| | | genome. |
| 279) | 17mers_human_m_virus_2MM_143_p.txt | Bombyx mori nuclear |
| | | polyhedrosis virus, complete genome. |
| 280) | 17mers_human_m_virus_2MM_1480_p.txt | Mycobacteriophage Omega |
| | | virion, complete genome. |
| 281) | 17mers_human_m_virus_2MM_142_p.txt | Autographa californica |
| | | nucleopolyhedrovirus, complete genome. |
| 282) | 17mers_human_m_virus_2MM_146_p.txt | Epiphyas postvittana |
| | | nucleopolyhedrovirus, complete genome. |
| 283) | 17mers_human_m_virus_2MM_156_p.txt | Xestia c-nigrum granulovirus, |
| | | complete genome. |
| 284) | 17mers_human_m_virus_2MM_151_p.txt | Mamestra configurata |
| | | nucleopolyhedrovirus B, complete genome. |
| 285) | 17mers_human_m_virus_2MM_152_p.txt | Orgyia pseudotsugata multicapsid |
| | | nucleopolyhedrovirus, complete genome. |
| 286) | 17mers_human_m_virus_2MM_267_p.txt | lctalurid herpesvirus 1, complete |
| | | genome. |
| 287) | 17mers_human_m_virus_2MM_145_p.txt | Culex nigripalpus baculovirus, |
| | | complete genome. |
| 288) | 17mers_human_m_virus_2MM_1476_p.txt | Mycobacteriophage Che9c virion, |
| | | complete genome. |
| 289) | 17mers_human_m_virus_2MM_144_p.txt | Choristoneura fumiferana MNPV, |
| | | complete genome. |
| 290) | 17mers_human_m_virus_2MM_150_p.txt | Mamestra configurata |
| | | nucleopolyhedrovirus A, complete genome. |
| 291) | 17mers_human_m_virus_2MM_147_p.txt | Helicoverpa armigera nuclear |
| | | polyhedrosis virus, complete genome. |
| 292) | 17mers_human_m_virus_2MM_149_p.txt | Heliocoverpa armigera |
| | | nucleopolyhedrovirus G4, complete genome. |
| 293) | 17mers_human_m_virus_2MM_148_p.txt | Helicoverpa zea single |
| | | nucleocapsid nucleopolyhedrovirus, complete genome. |
| 294) | 17mers_human_m_virus_2MM_184_p.txt | Bacteriophage phiE125, complete |
| | | genome. |
| 295) | 17mers_human_m_virus_2MM_195_p.txt | Burkholderia cepacia phage |
| | | Bcep781, complete genome. |
| 296) | 17mers_human_m_virus_2MM_406_p.txt | Rabbit fibroma virus, complete |
| | | genome. |
| 297) | 17mers_human_m_virus_2MM_258_p.txt | Gallid herpesvirus 3, complete |
| | | genome. |
| 298) | 17mers_human_m_virus_2MM_253_p.txt | Chimpanzee cytomegalovirus, |
| | | complete genome. |
| 299) | 17mers_human_m_virus_2MM_271_p.txt | Rat cytomegalovirus, complete |
| | | genome. |
| 300) | 17mers_human_m_virus_2MM_155_p.txt | Spodoptera exigua |
| | | nucleopolyhedrovirus, complete genome. |
| 301) | 17mers_human_m_virus_2MM_262_p.txt | Human herpesvirus 5, complete |
| | | genome. |
| 302) | 17mers_human_m_virus_2MM_243_p.txt | Virus PhiCh1, complete genome. |
| 303) | 17mers_human_m_virus_2MM_273_p.txt | Tupaia herpesvirus, complete |
| | | genome. |
| 304) | 17mers_human_m_virus_2MM_385_p.txt | Ectocarpus siliculosus virus, |
| | | complete genome. |
| 305) | 17mers_human_m_virus_2MM_269_p.txt | Mouse cytomegalovirus 1, |
| | | complete genome. |
| 306) | 17mers_human_m_virus_2MM_405_p.txt | Myxoma virus, complete genome. |

3) Virus 17 mers 1MM - It contains 1095 files, containing sequences of length 17,
which are present in the viral genome, but are not present in the human genome.
All sequences produced from these sequences with one mismatch are also not
present in the human genome. The list below contains the file name and the
corresponding virus name.

| | File Name | Species Taxonomy Name |
|---|---|---|
| 1) | 17mers_human_m_virus_1MM_1_p.txt | Hepatitis D virus, complete genome. |
| 2) | 17mers_human_m_virus_1MM_2_p.txt | Banana streak virus, complete genome. |
| 3) | 17mers_human_m_virus_1MM_3_p.txt | Blueberry red ringspot virus, complete genome. |
| 4) | 17mers_human_m_virus_1MM_4_p.txt | Cacao swollen shoot virus, complete genome. |
| 5) | 17mers_human_m_virus_1MM_5_p.txt | Carnation etched ring virus, complete genome. |
| 6) | 17mers_human_m_virus_1MM_6_p.txt | Cassava vein mosaic virus, complete genome. |
| 7) | 17mers_human_m_virus_1MM_7_p.txt | Cauliflower mosaic virus, complete genome. |
| 8) | 17mers_human_m_virus_1MM_8_p.txt | Cestrum yellow leaf curling virus, complete |
| | | genome. |
| 9) | 17mers_human_m_virus_1MM_9_p.txt | Citrus yellow mosaic virus, complete genome. |
| 10) | 17mers_human_m_virus_1MM_10_p.txt | Commelina yellow mottle virus, complete genome. |
| 11) | 17mers_human_m_virus_1MM_11_p.txt | Figwort mosaic virus, complete genome. |
| 12) | 17mers_human_m_virus_1MM_12_p.txt | Kalanchoe top-spotting virus, complete genome. |
| 13) | 17mers_human_m_virus_1MM_13_p.txt | Mirabilis mosaic virus, complete genome. |
| 14) | 17mers_human_m_virus_1MM_14_p.txt | Peanut chlorotic streak virus, complete genome. |
| 15) | 17mers_human_m_virus_1MM_15_p.txt | Petunia vein clearing virus, complete genome. |
| 16) | 17mers_human_m_virus_1MM_16_p.txt | Rice tungro bacilliform virus, complete genome. |
| 17) | 17mers_human_m_virus_1MM_17_p.txt | Soybean chlorotic mottle virus, complete genome. |
| 18) | 17mers_human_m_virus_1MM_18_p.txt | Strawberry vein banding virus, complete genome. |
| 19) | 17mers_human_m_virus_1MM_19_p.txt | Sugarcane bacilliform virus, complete genome. |
| 20) | 17mers_human_m_virus_1MM_20_p.txt | Taro bacilliform virus, complete genome. |
| 21) | 17mers_human_m_virus_1MM_21_p.txt | Tobacco vein-clearing virus, complete genome. |
| 22) | 17mers_human_m_virus_1MM_22_p.txt | Arctic ground squirrel hepatitis B virus, complete |
| | | genome. |
| 23) | 17mers_human_m_virus_1MM_23_p.txt | Duck hepatitis B virus, complete genome. |
| 24) | 17mers_human_m_virus_1MM_24_p.txt | Ground squirrel hepatitis virus, complete genome. |
| 25) | 17mers_human_m_virus_1MM_25_p.txt | Hepatitis B virus, complete genome. |
| 26) | 17mers_human_m_virus_1MM_26_p.txt | Heron hepatitis B virus, complete genome. |
| 27) | 17mers_human_m_virus_1MM_27_p.txt | Orangutan hepadnavirus, complete genome. |
| 28) | 17mers_human_m_virus_1MM_28_p.txt | Woodchuck hepatitis B virus, complete genome. |
| 29) | 17mers_human_m_virus_1MM_29_p.txt | Woolly monkey hepatitis B Virus, complete |
| | | genome. |
| 30) | 17mers_human_m_virus_1MM_30_p.txt | Abelson murine leukemia virus, complete genome. |
| 31) | 17mers_human_m_virus_1MM_31_p.txt | Avian carcinoma virus, complete genome. |
| 32) | 17mers_human_m_virus_1MM_32_p.txt | Avian leukosis virus, complete genome. |
| 33) | 17mers_human_m_virus_1MM_33_p.txt | Avian myelocytomatosis virus, complete genome. |
| 34) | 17mers_human_m_virus_1MM_34_p.txt | Avian sarcoma virus, complete genome. |
| 35) | 17mers_human_m_virus_1MM_35_p.txt | Bovine foamy virus, complete genome. |
| 36) | 17mers_human_m_virus_1MM_36_p.txt | Bovine immunodeficiency virus, complete genome. |
| 37) | 17mers_human_m_virus_1MM_37_p.txt | Bovine leukemia virus, complete genome. |
| 38) | 17mers_human_m_virus_1MM_38_p.txt | Caprine arthritis-encephalitis virus, complete |
| | | genome. |
| 39) | 17mers_human_m_virus_1MM_39_p.txt | Equine foamy virus, complete genome. |
| 40) | 17mers_human_m_virus_1MM_40_p.txt | Equine infectious anemia virus, complete genome. |
| 41) | 17mers_human_m_virus_1MM_41_p.txt | Feline foamy virus, complete genome. |
| 42) | 17mers_human_m_virus_1MM_42_p.txt | Feline immunodeficiency virus, complete genome. |
| 43) | 17mers_human_m_virus_1MM_43_p.txt | Feline leukemia virus, complete genome. |
| 44) | 17mers_human_m_virus_1MM_44_p.txt | Friend murine leukemia virus, complete genome. |
| 45) | 17mers_human_m_virus_1MM_45_p.txt | Fujinami sarcoma virus, complete genome. |
| 46) | 17mers_human_m_virus_1MM_46_p.txt | Gibbon ape leukemia virus, complete genome. |
| 47) | 17mers_human_m_virus_1MM_47_p.txt | Human foamy virus, complete genome. |
| 48) | 17mers_human_m_virus_1MM_48_p.txt | Human immunodeficiency virus 1, complete |
| | | genome. |
| 49) | 17mers_human_m_virus_1MM_49_p.txt | Human immunodeficiency virus 2, complete |
| | | genome. |
| 50) | 17mers_human_m_virus_1MM_50_p.txt | Human spumaretrovirus, complete genome. |
| 51) | 17mers_human_m_virus_1MM_51_p.txt | Human T-lymphotropic virus 1, complete genome. |
| 52) | 17mers_human_m_virus_1MM_52_p.txt | Human T-lymphotropic virus 2, complete genome. |
| 53) | 17mers_human_m_virus_1MM_53_p.txt | Jembrana disease virus, complete genome. |
| 54) | 17mers_human_m_virus_1MM_54_p.txt | Mason-Pfizer monkey virus, complete genome. |
| 55) | 17mers_human_m_virus_1MM_55_p.txt | Moloney murine sarcoma virus, complete genome. |
| 56) | 17mers_human_m_virus_1MM_56_p.txt | Mouse mammary tumor virus, complete genome. |
| 57) | 17mers_human_m_virus_1MM_57_p.txt | Murine leukemia virus, complete genome. |
| 58) | 17mers_human_m_virus_1MM_58_p.txt | Murine osteosarcoma virus, complete genome. |
| 59) | 17mers_human_m_virus_1MM_59_p.txt | Murine sarcoma virus, complete genome. |
| 60) | 17mers_human_m_virus_1MM_60_p.txt | Murine type C retrovirus, complete genome. |
| 61) | 17mers_human_m_virus_1MM_61_p.txt | Ovine lentivirus, complete genome. |
| 62) | 17mers_human_m_virus_1MM_62_p.txt | Ovine pulmonary adenocarcinoma virus, complete |
| | | genome. |
| 63) | 17mers_human_m_virus_1MM_63_p.txt | Porcine endogenous retrovirus, complete genome. |
| 64) | 17mers_human_m_virus_1MM_64_p.txt | Rauscher murine leukemia virus, complete genome. |
| 65) | 17mers_human_m_virus_1MM_65_p.txt | Rous sarcoma virus, complete genome. |
| 66) | 17mers_human_m_virus_1MM_66_p.txt | Simian foamy virus, complete genome. |
| 67) | 17mers_human_m_virus_1MM_67_p.txt | Simian immunodeficiency virus, complete genome. |
| 68) | 17mers_human_m_virus_1MM_68_p.txt | Simian immunodeficiency virus 2, complete |
| | | genome. |
| 69) | 17mers_human_m_virus_1MM_69_p.txt | Simian T-lymphotropic virus 1, complete genome. |
| 70) | 17mers_human_m_virus_1MM_70_p.txt | Simian T-lymphotropic virus 2, complete genome. |
| 71) | 17mers_human_m_virus_1MM_71_p.txt | Simian-Human immunodeficiency virus, complete |
| | | genome. |
| 72) | 17mers_human_m_virus_1MM_72_p.txt | Snakehead retrovirus, complete genome. |
| 73) | 17mers_human_m_virus_1MM_73_p.txt | Spleen focus-forming virus, complete genome. |
| 74) | 17mers_human_m_virus_1MM_74_p.txt | Visna virus, complete genome. |
| 75) | 17mers_human_m_virus_1MM_75_p.txt | Walleye dermal sarcoma virus, complete genome. |
| 76) | 17mers_human_m_virus_1MM_76_p.txt | Woolly monkey sarcoma virus, complete genome. |
| 77) | 17mers_human_m_virus_1MM_77_p.txt | Y73 sarcoma virus, complete genome. |
| 78) | 17mers_human_m_virus_1MM_78_p.txt | Ageratum yellow vein virus-associated DNA beta, |
| | | complete genome. |
| 79) | 17mers_human_m_virus_1MM_79_p.txt | Arabis mosaic virus satellite RNA, complete |
| | | genome. |
| 80) | 17mers_human_m_virus_1MM_80_p.txt | Arabis mosaic virus small satellite RNA, complete |
| | | genome. |
| 81) | 17mers_human_m_virus_1MM_81_p.txt | Bamboo mosaic virus satellite RNA, complete |
| | | genome. |
| 82) | 17mers_human_m_virus_1MM_82_p.txt | Bhendi yellow vein mosaic virus-associated DNA |
| | | beta, complete genome. |
| 83) | 17mers_human_m_virus_1MM_83_p.txt | Cereal yellow dwarf virus-RPV satellite RNA, |
| | | complete genome. |
| 84) | 17mers_human_m_virus_1MM_84_p.txt | Chicory yellow mottle virus large satellite RNA, |
| | | complete genome. |
| 85) | 17mers_human_m_virus_1MM_85_p.txt | Chicory yellow mottle virus satellite RNA, |
| | | complete genome. |
| 86) | 17mers_human_m_virus_1MM_86_p.txt | Cotton leaf curl virus-associated DNA beta, |
| | | complete genome. |
| 87) | 17mers_human_m_virus_1MM_87_p.txt | Cucumber mosaic virus satellite RNA, complete |
| | | genome. |
| 88) | 17mers_human_m_virus_1MM_88_p.txt | Cymbidium ringspot virus satellite RNA, complete |
| | | genome. |
| 89) | 17mers_human_m_virus_1MM_89_p.txt | Eupatorium yellow vein virus-associated DNA beta, |
| | | complete genome. |
| 90) | 17mers_human_m_virus_1MM_90_p.txt | Grapevine fanleaf virus satellite RNA, complete |
| | | genome. |
| 91) | 17mers_human_m_virus_1MM_91_p.txt | Hollyhock leaf crumple virus satellite DNA, |
| | | complete genome. |
| 92) | 17mers_human_m_virus_1MM_92_p.txt | Lucerne transient streak virus satellite RNA, |
| | | complete genome. |
| 93) | 17mers_human_m_virus_1MM_93_p.txt | Malvastrum yellow vein virus-associated DNA beta, |
| | | complete genome. |
| 94) | 17mers_human_m_virus_1MM_94_p.txt | OkLCV satDNA 10, complete genome. |
| 95) | 17mers_human_m_virus_1MM_95_p.txt | OkLCV satDNA 27, complete genome. |
| 96) | 17mers_human_m_virus_1MM_96_p.txt | OkLCV satDNA 3, complete genome. |
| 97) | 17mers_human_m_virus_1MM_97_p.txt | Panicum mosaic satellite virus, complete genome. |
| 98) | 17mers_human_m_virus_1MM_98_p.txt | Papaya leaf curl virus-associated DNA beta, |
| | | complete genome. |
| 99) | 17mers_human_m_virus_1MM_99_p.txt | Pea enation mosaic virus satellite RNA, complete |
| | | genome. |
| 100) | 17mers_human_m_virus_1MM_100_p.txt | Peanut stunt virus satellite RNA, complete |
| | | genome. |
| 101) | 17mers_human_m_virus_1MM_101_p.txt | Rice yellow mottle virus satellite, complete |
| | | genome. |
| 102) | 17mers_human_m_virus_1MM_102_p.txt | Satellite virus of maize white line mosaic |
| | | virus, complete genome. |
| 103) | 17mers_human_m_virus_1MM_103_p.txt | Satellite of Trichomonas vaginalis T1 |
| | | virus, complete genome. |
| 104) | 17mers_human_m_virus_1MM_104_p.txt | Satellite RNA of Blackcurrant reversion |
| | | virus, complete genome. |
| 105) | 17mers_human_m_virus_1MM_105_p.txt | Satellite RNA of Groundnut Rosette Virus, |
| | | complete genome. |
| 106) | 17mers_human_m_virus_1MM_106_p.txt | Satellite tobacco mosaic virus, complete |
| | | genome. |
| 107) | 17mers_human_m_virus_1MM_107_p.txt | Solanum nodiflorum mottle virus satellite |
| | | RNA, complete genome. |
| 108) | 17mers_human_m_virus_1MM_108_p.txt | Strawberry latent ringspot virus satellite |
| | | RNA, complete genome. |
| 109) | 17mers_human_m_virus_1MM_109_p.txt | Subterranean clover mottle virus satellite |
| | | RNA, complete genome. |
| 110) | 17mers_human_m_virus_1MM_110_p.txt | Tobacco curly shoot virus- associated DNA |
| | | beta, complete genome. |
| 111) | 17mers_human_m_virus_1MM_111_p.txt | Tobacco necrosis satellite virus, complete |
| | | genome. |
| 112) | 17mers_human_m_virus_1MM_112_p.txt | Tobacco ringspot virus satellite RNA, |
| | | complete genome. |
| 113) | 17mers_human_m_virus_1MM_113_p.txt | Tomato black ring virus satellite RNA, |
| | | complete genome. |
| 114) | 17mers_human_m_virus_1MM_114_p.txt | Tomato bushy stunt virus satellite RNA |
| | | B1, complete genome. |
| 115) | 17mers_human_m_virus_1MM_115_p.txt | Tomato bushy stunt virus satellite RNA |
| | | B10, complete genome. |
| 116) | 17mers_human_m_virus_1MM_116_p.txt | Tomato leaf curl virus satellite DNA, |
| | | complete genome. |
| 117) | 17mers_human_m_virus_1MM_117_p.txt | Tomato leaf curl virus- associated DNA |
| | | beta, complete genome. |
| 118) | 17mers_human_m_virus_1MM_118_p.txt | Tomato yellow leaf curl China virus- |
| | | associated DNA beta, complete genome. |
| 119) | 17mers_human_m_virus_1MM_119_p.txt | Turnip crinkle virus satellite RNA, |
| | | complete genome. |
| 120) | 17mers_human_m_virus_1MM_120_p.txt | Velvet tobacco mottle virus Satellite RNA, |
| | | complete genome. |
| 121) | 17mers_human_m_virus_1MM_121_p.txt | Bovine adenovirus B, complete genome. |
| 122) | 17mers_human_m_virus_1MM_122_p.txt | Bovine adenovirus D, complete genome. |
| 123) | 17mers_human_m_virus_1MM_123_p.txt | Canine adenovirus type 1, complete |
| | | genome. |
| 124) | 17mers_human_m_virus_1MM_124_p.txt | Duck adenovirus 1, complete genome. |
| 125) | 17mers_human_m_virus_1MM_125_p.txt | Fowl adenovirus A, complete genome. |
| 126) | 17mers_human_m_virus_1MM_126_p.txt | Fowl adenovirus D, complete genome. |
| 127) | 17mers_human_m_virus_1MM_127_p.txt | Frog adenovirus 1, complete genome. |
| 128) | 17mers_human_m_virus_1MM_128_p.txt | Human adenovirus A, complete genome. |
| 129) | 17mers_human_m_virus_1MM_129_p.txt | Human adenovirus B, complete genome. |
| 130) | 17mers_human_m_virus_1MM_130_p.txt | Human adenovirus C, complete genome. |
| 131) | 17mers_human_m_virus_1MM_131_p.txt | Human adenovirus D, complete genome. |
| 132) | 17mers_human_m_virus_1MM_132_p.txt | Human adenovirus E, complete genome. |
| 133) | 17mers_human_m_virus_1MM_133_p.txt | Human adenovirus F, complete genome. |
| 134) | 17mers_human_m_virus_1MM_134_p.txt | Murine adenovirus A, complete genome. |
| 135) | 17mers_human_m_virus_1MM_135_p.txt | Ovine adenovirus 7, complete genome. |
| 136) | 17mers_human_m_virus_1MM_136_p.txt | Ovine adenovirus A, complete genome. |
| 137) | 17mers_human_m_virus_1MM_137_p.txt | Porcine adenovirus A, complete genome. |
| 138) | 17mers_human_m_virus_1MM_138_p.txt | Porcine adenovirus C, complete genome. |
| 139) | 17mers_human_m_virus_1MM_139_p.txt | Tupaia adenovirus I, complete genome. |
| 140) | 17mers_human_m_virus_1MM_140_p.txt | Hemorrhagic enteritis virus, complete |
| | | genome. |
| 141) | 17mers_human_m_virus_1MM_141_p.txt | Adoxophyes honmai |
| | | nucleopolyhedrovirus, complete genome. |
| 142) | 17mers_human_m_virus_1MM_142_p.txt | Autographa californica |
| | | nucleopolyhedrovirus, complete genome. |
| 143) | 17mers_human_m_virus_1MM_143_p.txt | Bombyx mori nuclear polyhedrosis virus, |
| | | complete genome. |
| 144) | 17mers_human_m_virus_1MM_144_p.txt | Choristoneura fumiferana MNPV, |
| | | complete genome. |
| 145) | 17mers_human_m_virus_1MM_145_p.txt | Culex nigripalpus baculovirus, complete |
| | | genome. |
| 146) | 17mers_human_m_virus_1MM_146_p.txt | Epiphyas postvittana |
| | | nucleopolyhedrovirus, complete genome. |
| 147) | 17mers_human_m_virus_1MM_147_p.txt | Helicoverpa armigera nuclear polyhedrosis |
| | | virus, complete genome. |
| 148) | 17mers_human_m_virus_1MM_148_p.txt | Helicoverpa zea single nucleocapsid |
| | | nucleopolyhedrovirus, complete genome. |
| 149) | 17mers_human_m_virus_1MM_149_p.txt | Heliocoverpa armigera |
| | | nucleopolyhedrovirus G4, complete genome. |
| 150) | 17mers_human_m_virus_1MM_150_p.txt | Mamestra configurata |
| | | nucleopolyhedrovirus A, complete genome. |
| 151) | 17mers_human_m_virus_1MM_151_p.txt | Mamestra configurata |
| | | nucleopolyhedrovirus B, complete genome. |
| 152) | 17mers_human_m_virus_1MM_152_p.txt | Orgyia pseudotsugata multicapsid |
| | | nucleopolyhedrovirus, complete genome. |
| 153) | 17mers_human_m_virus_1MM_153_p.txt | Plutella xylostella granulovirus, complete |
| | | genome. |
| 154) | 17mers_human_m_virus_1MM_154_p.txt | Rachiplusia ou multiple |
| | | nucleopolyhedrovirus, complete genome. |
| 155) | 17mers_human_m_virus_1MM_155_p.txt | Spodoptera exigua nucleopolyhedrovirus, |
| | | complete genome. |
| 156) | 17mers_human_m_virus_1MM_156_p.txt | Xestia c-nigrum granulovirus, complete |
| | | genome. |
| 157) | 18mers_human_m_virus_1MM_157_p.txt | Acyrthosiphon pisum bacteriophage APSE-1, complete |
| | | genome. |
| 158) | 18mers_human_m_virus_1MM_158_p.txt | Bacillus phage GA-1 virion, complete genome. |
| 159) | 18mers_human_m_virus_1MM_159_p.txt | Bacillus phage PZA, complete genome. |
| 160) | 18mers_human_m_virus_1MM_160_p.txt | Bacteriophage 933W provirus, complete genome. |
| 161) | 18mers_human_m_virus_1MM_161_p.txt | Bacteriophage A118 virion, complete genome. |
| 162) | 18mers_human_m_virus_1MM_162_p.txt | Bacteriophage B103, complete genome. |
| 163) | 18mers_human_m_virus_1MM_163_p.txt | Bacteriophage bIL170, complete genome. |
| 164) | 18mers_human_m_virus_1MM_164_p.txt | Bacteriophage bIL285, complete genome. |
| 165) | 18mers_human_m_virus_1MM_165_p.txt | Bacteriophage bIL286, complete genome. |
| 166) | 18mers_human_m_virus_1MM_166_p.txt | Bacteriophage bIL309, complete genome. |
| 167) | 18mers_human_m_virus_1MM_167_p.txt | Bacteriophage bIL310, complete genome. |
| 168) | 18mers_human_m_virus_1MM_168_p.txt | Bacteriophage bIL311, complete genome. |
| 169) | 18mers_human_m_virus_1MM_169_p.txt | Bacteriophage bIL312, complete genome. |
| 170) | 18mers_human_m_virus_1MM_170_p.txt | Bacteriophage HK620, complete genome. |
| 171) | 18mers_human_m_virus_1MM_171_p.txt | Bacteriophage HK97, complete genome. |
| 172) | 18mers_human_m_virus_1MM_172_p.txt | Bacteriophage K139, complete genome. |
| 173) | 18mers_human_m_virus_1MM_173_p.txt | Bacteriophage L-413C, complete genome. |
| 174) | 18mers_human_m_virus_1MM_174_p.txt | Bacteriophage lambda, complete genome. |
| 175) | 18mers_human_m_virus_1MM_175_p.txt | Bacteriophage N15 virion, complete genome. |
| 176) | 18mers_human_m_virus_1MM_176_p.txt | Bacteriophage P27, complete genome. |
| 177) | 18mers_human_m_virus_1MM_177_p.txt | Bacteriophage P4, complete genome. |
| 178) | 18mers_human_m_virus_1MM_178_p.txt | Bacteriophage phi CTX, complete genome. |
| 179) | 18mers_human_m_virus_1MM_179_p.txt | Bacteriophage phi ETA, complete genome. |
| 180) | 18mers_human_m_virus_1MM_180_p.txt | Bacteriophage phi-105, complete genome. |
| 181) | 18mers_human_m_virus_1MM_181_p.txt | Bacteriophage phi-BT1 virion, complete genome. |
| 182) | 18mers_human_m_virus_1MM_182_p.txt | Bacteriophage phi-C31, complete genome. |
| 183) | 18mers_human_m_virus_1MM_183_p.txt | Bacteriophage phi3626, complete genome. |
| 184) | 18mers_human_m_virus_1MM_184_p.txt | Bacteriophage phiE125, complete genome. |
| 185) | 18mers_human_m_virus_1MM_185_p.txt | Bacteriophage phig1e, complete genome. |
| 186) | 18mers_human_m_virus_1MM_186_p.txt | Bacteriophage phiYeO3-12, complete genome. |
| 187) | 18mers_human_m_virus_1MM_187_p.txt | Bacteriophage rlt, complete genome. |
| 188) | 18mers_human_m_virus_1MM_188_p.txt | Bacteriophage sk1, complete genome. |
| 189) | 18mers_human_m_virus_1MM_189_p.txt | Bacteriophage SPBc2, complete genome. |
| 190) | 18mers_human_m_virus_1MM_190_p.txt | Bacteriophage SPP1, complete genome. |
| 191) | 18mers_human_m_virus_1MM_191_p.txt | Bacteriophage T3, complete genome. |
| 192) | 18mers_human_m_virus_1MM_192_p.txt | Bacteriophage TP901-1, complete genome. |
| 193) | 18mers_human_m_virus_1MM_193_p.txt | Bacteriophage Tuc2009, complete genome. |
| 194) | 18mers_human_m_virus_1MM_194_p.txt | Bacteriophage VT2-Sa provirus, complete genome. |
| 195) | 18mers_human_m_virus_1MM_195_p.txt | Burkholderia cepacia phage Bcep781, complete genome. |
| 196) | 18mers_human_m_virus_1MM_196_p.txt | Enterobacteria phage 186, complete genome. |
| 197) | 18mers_human_m_virus_1MM_197_p.txt | Enterobacteria phage epsilon15, complete genome. |
| 198) | 18mers_human_m_virus_1MM_198_p.txt | Enterobacteria phage HK022 virion, complete genome. |
| 199) | 18mers_human_m_virus_1MM_199_p.txt | Enterobacteria phage Mu, complete genome. |
| 200) | 18mers_human_m_virus_1MM_200_p.txt | Enterobacteria phage P2, complete genome. |
| 201) | 18mers_human_m_virus_1MM_201_p.txt | Enterobacteria phage T4, complete genome. |
| 202) | 18mers_human_m_virus_1MM_202_p.txt | Enterobacteria phage T7. |
| 203) | 18mers_human_m_virus_1MM_203_p.txt | Haemophilus phage HP1, complete genome. |
| 204) | 18mers_human_m_virus_1MM_204_p.txt | Haemophilus phage HP2, complete genome. |
| 205) | 18mers_human_m_virus_1MM_205_p.txt | Lactobacillus bacteriophage phi adh, complete genome. |
| 206) | 18mers_human_m_virus_1MM_206_p.txt | Lactobacillus casei bacteriophage A2 virion, complete |
| | | genome. |
| 207) | 18mers_human_m_virus_1MM_207_p.txt | Lactococcus lactis bacteriophage u136, complete genome. |
| 208) | 18mers_human_m_virus_1MM_208_p.txt | Lactococcus phage BK5-T, complete genome. |
| 209) | 18mers_human_m_virus_1MM_209_p.txt | Lactococcus phage c2. |
| 210) | 18mers_human_m_virus_1MM_210_p.txt | Lactococcus phage P335, complete genome. |
| 211) | 18mers_human_m_virus_1MM_211_p.txt | Listeria phage 2389 virion, complete genome. |
| 212) | 18mers_human_m_virus_1MM_212_p.txt | Methanobacterium phage psiM2, complete genome. |
| 213) | 18mers_human_m_virus_1MM_213_p.txt | Methanothermobacter wolfeii prophage psiM100. |
| | | complete genome. |
| 214) | 18mers_human_m_virus_1MM_214_p.txt | Mycobacteriophage Bxb1, complete genome. |
| 215) | 18mers_human_m_virus_1MM_215_p.txt | Mycobacteriophage D29, complete genome. |
| 216) | 18mers_human_m_virus_1MM_216_p.txt | Mycobacteriophage TM4, complete genome. |
| 217) | 18mers_human_m_virus_1MM_217_p.txt | Mycobacterium phage L5. |
| 218) | 18mers_human_m_virus_1MM_218_p.txt | Mycoplasma virus P1, complete genome. |
| 219) | 18mers_human_m_virus_1MM_219_p.txt | Pseudomonas aeruginosa phage PaP3, complete genome. |
| 220) | 18mers_human_m_virus_1MM_220_p.txt | Pseudomonas phage D3, complete genome. |
| 221) | 18mers_human_m_virus_1MM_221_p.txt | Pseudomonas phage gh-1, complete genome. |
| 222) | 18mers_human_m_virus_1MM_222_p.txt | Pseudomonas phage phiKZ, complete genome. |
| 223) | 18mers_human_m_virus_1MM_223_p.txt | Roseophage SIO1, complete genome. |
| 224) | 18mers_human_m_virus_1MM_224_p.txt | Salmonella typhimurium bacteriophage ST64T virion, |
| | | complete genome. |
| 225) | 18mers_human_m_virus_1MM_225_p.txt | Salmonella typhimurium phage ST64B, complete |
| | | genome. |
| 226) | 18mers_human_m_virus_1MM_226_p.txt | Shigella flexneri bacteriophage V, complete genome. |
| 227) | 18mers_human_m_virus_1MM_227_p.txt | Staphylococcus aureus bacteriophage PVL provirus, |
| | | complete genome. |
| 228) | 18mers_human_m_virus_1MM_228_p.txt | Staphylococcus aureus phage phiP68, complete genome. |
| 229) | 18mers_human_m_virus_1MM_229_p.txt | Staphylococcus aureus prophage phiPV83 provirus, |
| | | complete genome. |
| 230) | 18mers_human_m_virus_1MM_230_p.txt | Staphylococcus aureus temperate phage phiSLT virion, |
| | | complete genome. |
| 231) | 18mers_human_m_virus_1MM_231_p.txt | Staphylococcus phage 44AHJD, complete genome. |
| 232) | 18mers_human_m_virus_1MM_232_p.txt | Streptococcus phage Cp-1, complete genome. |
| 233) | 18mers_human_m_virus_1MM_233_p.txt | Streptococcus thermophilus bacteriophage 7201, complete |
| | | genome. |
| 234) | 18mers_human_m_virus_1MM_234_p.txt | Streptococcus thermophilus bacteriophage DT1, complete |
| | | genome. |
| 235) | 18mers_human_m_virus_1MM_235_p.txt | Streptococcus thermophilus bacteriophage Sfi11, |
| | | complete genome. |
| 236) | 18mers_human_m_virus_1MM_236_p.txt | Streptococcus thermophilus bacteriophage Sfi19, |
| | | complete genome. |
| 237) | 18mers_human_m_virus_1MM_237_p.txt | Streptococcus thermophilus bacteriophage Sfi21, |
| | | complete genome. |
| 238) | 18mers_human_m_virus_1MM_238_p.txt | Streptococcus thermophilus temperate bacteriophage |
| | | O1205, complete genome. |
| 239) | 18mers_human_m_virus_1MM_239_p.txt | Stx2 converting bacteriophage 1 virion, complete genome. |
| 240) | 18mers_human_m_virus_1MM_240_p.txt | Temperate phage PhiN1H1.1, complete genome. |
| 241) | 18mers_human_m_virus_1MM_241_p.txt | Vibrio harveyi bacteriophage VHML, complete genome. |
| 242) | 18mers_human_m_virus_1MM_242_p.txt | Vibriophage VpV262, complete genome. |
| 243) | 18mers_human_m_virus_1MM_243_p.txt | Virus PhiCh1, complete genome. |
| 244) | 18mers_human_m_virus_1MM_244_p.txt | Yersinia pestis phage phiA1122, complete genome. |
| 245) | 18mers_human_m_virus_1MM_245_p.txt | Alteromonas phage PM2, complete genome. |
| 246) | 18mers_human_m_virus_1MM_246_p.txt | Sulfolobus virus 1, complete genome. |
| 247) | 18mers_human_m_virus_1MM_247_p.txt | Alcelaphine herpesvirus 1, complete genome. |
| 248) | 18mers_human_m_virus_1MM_248_p.txt | Ateline herpesvirus 3, complete genome. |
| 249) | 18mers_human_m_virus_1MM_249_p.txt | Bovine herpesvirus 1, complete genome. |
| 250) | 18mers_human_m_virus_1MM_250_p.txt | Bovine herpesvirus 4, complete genome. |
| 251) | 18mers_human_m_virus_1MM_251_p.txt | Callitrichine herpesvirus 3, complete genome. |
| 252) | 18mers_human_m_virus_1MM_252_p.txt | Cercopithecine herpesvirus 7, complete genome. |
| 253) | 18mers_human_m_virus_1MM_253_p.txt | Chimpanzee cytomegalovirus, complete genome. |
| 254) | 18mers_human_m_virus_1MM_254_p.txt | Equine herpesvirus 1, complete genome. |
| 255) | 18mers_human_m_virus_1MM_255_p.txt | Equine herpesvirus 2, complete genome. |
| 256) | 18mers_human_m_virus_1MM_256_p.txt | Equine herpesvirus 4, complete genome. |
| 257) | 18mers_human_m_virus_1MM_257_p.txt | Gallid herpesvirus 2, complete genome. |
| 258) | 18mers_human_m_virus_1MM_258_p.txt | Gallid herpesvirus 3, complete genome. |
| 259) | 18mers_human_m_virus_1MM_259_p.txt | Human herpesvirus 2, complete genome. |
| 260) | 18mers_human_m_virus_1MM_260_p.txt | Human herpesvirus 3, complete genome. |
| 261) | 18mers_human_m_virus_1MM_261_p.txt | Human herpesvirus 4, complete genome. |
| 262) | 18mers_human_m_virus_1MM_262_p.txt | Human herpesvirus 5, complete genome. |
| 263) | 18mers_human_m_virus_1MM_263_p.txt | Human herpesvirus 6, complete genome. |
| 264) | 18mers_human_m_virus_1MM_264_p.txt | Human herpesvirus 6B, complete genome. |
| 265) | 18mers_human_m_virus_1MM_265_p.txt | Human herpesvirus 7, complete genome. |
| 266) | 18mers_human_m_virus_1MM_266_p.txt | Human herpesvirus 8, genome. |
| 267) | 18mers_human_m_virus_1MM_267_p.txt | Ictalurid herpesvirus 1, complete genome. |
| 268) | 18mers_human_m_virus_1MM_268_p.txt | Meleagrid herpesvirus 1, complete genome. |
| 269) | 18mers_human_m_virus_1MM_269_p.txt | Mouse cytomegalovirus 1, complete genome. |
| 270) | 18mers_human_m_virus_1MM_270_p.txt | Murid herpesvirus 4, complete genome. |
| 271) | 18mers_human_m_virus_1MM_271_p.txt | Rat cytomegalovirus, complete genome. |
| 272) | 18mers_human_m_virus_1MM_272_p.txt | Saimiriine herpesvirus 2, complete genome. |
| 273) | 18mers_human_m_virus_1MM_273_p.txt | Tupaia herpesvirus, complete genome. |
| 274) | 18mers_human_m_virus_1MM_274_p.txt | Infectious spleen and kidney necrosis virus, complete |
| | | genome. |
| 275) | 18mers_human_m_virus_1MM_275_p.txt | Invertebrate iridescent virus 6, complete genome. |
| 276) | 18mers_human_m_virus_1MM_276_p.txt | Lymphocystis disease virus 1, complete genome. |
| 277) | 18mers_human_m_virus_1MM_277_p.txt | Rana tigrina ranavirus, complete genome. |
| 278) | 18mers_human_m_virus_1MM_278_p.txt | Sulfolobus islandicus filamentous virus, genome. |
| 279) | 18mers_human_m_virus_1MM_279_p.txt | Shrimp white spot syndrome virus, complete genome. |
| 280) | 18mers_human_m_virus_1MM_280_p.txt | Bovine papillomavirus, complete genome. |
| 281) | 18mers_human_m_virus_1MM_281_p.txt | Bovine papillomavirus type 2, complete genome. |
| 282) | 18mers_human_m_virus_1MM_282_p.txt | Bovine papillomavirus type 3, complete genome. |
| 283) | 18mers_human_m_virus_1MM_283_p.txt | Bovine papillomavirus type 4, complete genome. |
| 284) | 18mers_human_m_virus_1MM_284_p.txt | Bovine papillomavirus type 5, complete genome. |
| 285) | 18mers_human_m_virus_1MM_285_p.txt | Canine oral papillomavirus, complete genome. |
| 286) | 18mers_human_m_virus_1MM_286_p.txt | Common chimpanzee papillomavirus I, complete |
| | | genome. |
| 287) | 18mers_human_m_virus_1MM_287_p.txt | Cottontail rabbit papillomavirus, complete genome. |
| 288) | 18mers_human_m_virus_1MM_288_p.txt | Deer papillomavirus, complete genome. |
| 289) | 18mers_human_m_virus_1MM_289_p.txt | Equinus papillomavirus, complete genome. |
| 290) | 18mers_human_m_virus_1MM_290_p.txt | Equus caballus papillomavirus type 1, complete genome. |
| 291) | 18mers_human_m_virus_1MM_291_p.txt | European elk papillomavirus, complete genome. |
| 292) | 18mers_human_m_virus_1MM_292_p.txt | Feline papillomavirus, complete genome. |
| 293) | 18mers_human_m_virus_1MM_293_p.txt | Fringilla coelebs papillomavirus, complete genome. |
| 294) | 18mers_human_m_virus_1MM_294_p.txt | Hamster papovavirus, complete genome. |
| 295) | 18mers_human_m_virus_1MM_295_p.txt | Human papillomavirus candHPV85, complete genome. |
| 296) | 18mers_human_m_virus_1MM_296_p.txt | Human papillomavirus RTRX7, complete genome. |
| 297) | 18mers_human_m_virus_1MM_297_p.txt | Human papillomavirus type 10, complete genome. |
| 298) | 18mers_human_m_virus_1MM_298_p.txt | Human papillomavirus type 11, complete genome. |
| 299) | 18mers_human_m_virus_1MM_299_p.txt | Human papillomavirus type 12, complete genome. |
| 300) | 18mers_human_m_virus_1MM_300_p.txt | Human papillomavirus type 13, complete genome. |
| 301) | 18mers_human_m_virus_1MM_301_p.txt | Human papillomavirus type 14D, complete genome. |
| 302) | 18mers_human_m_virus_1MM_302_p.txt | Human papillomavirus type 15, complete genome. |
| 303) | 18mers_human_m_virus_1MM_303_p.txt | Human papillomavirus type 16, complete genome. |
| 304) | 18mers_human_m_virus_1MM_304_p.txt | Human papillomavirus type 17, complete genome. |
| 305) | 18mers_human_m_virus_1MM_305_p.txt | Human papillomavirus type 18, complete genome. |
| 306) | 18mers_human_m_virus_1MM_306_p.txt | Human papillomavirus type 19, complete genome. |
| 307) | 18mers_human_m_virus_1MM_307_p.txt | Human papillomavirus type 1a, complete genome. |
| 308) | 18mers_human_m_virus_1MM_308_p.txt | Human papillomavirus type 20, complete genome. |
| 309) | 18mers_human_m_virus_1MM_309_p.txt | Human papillomavirus type 21, complete genome. |
| 310) | 18mers_human_m_virus_1MM_310_p.txt | Human papillomavirus type 22, complete genome. |
| 311) | 18mers_human_m_virus_1MM_311_p.txt | Human papillomavirus type 23, complete genome. |
| 312) | 18mers_human_m_virus_1MM_312_p.txt | Human papillomavirus type 24, complete genome. |
| 313) | 18mers_human_m_virus_1MM_313_p.txt | Human papillomavirus type 25, complete genome. |
| 314) | 18mers_human_m_virus_1MM_314_p.txt | Human papillomavirus type 26, complete genome. |
| 315) | 18mers_human_m_virus_1MM_315_p.txt | Human papillomavirus type 27, complete genome. |
| 316) | 18mers_human_m_virus_1MM_316_p.txt | Human papillomavirus type 28, complete genome. |
| 317) | 18mers_human_m_virus_1MM_317_p.txt | Human papillomavirus type 29, complete genome. |
| 318) | 18mers_human_m_virus_1MM_318_p.txt | Human papillomavirus type 2a, complete genome. |
| 319) | 18mers_human_m_virus_1MM_319_p.txt | Human papillomavirus type 3, complete genome. |
| 320) | 18mers_human_m_virus_1MM_320_p.txt | Human papillomavirus type 30, complete genome. |
| 321) | 18mers_human_m_virus_1MM_321_p.txt | Human papillomavirus type 31, complete genome. |
| 322) | 18mers_human_m_virus_1MM_322_p.txt | Human papillomavirus type 32, complete genome. |
| 323) | 18mers_human_m_virus_1MM_323_p.txt | Human papillomavirus type 33, complete genome. |
| 324) | 18mers_human_m_virus_1MM_324_p.txt | Human papillomavirus type 34, complete genome. |
| 325) | 18mers_human_m_virus_1MM_325_p.txt | Human papillomavirus type 35, complete genome. |
| 326) | 18mers_human_m_virus_1MM_326_p.txt | Human papillomavirus type 36, complete genome. |
| 327) | 18mers_human_m_virus_1MM_327_p.txt | Human papillomavirus type 37, complete genome. |
| 328) | 18mers_human_m_virus_1MM_328_p.txt | Human papillomavirus type 38, complete genome. |
| 329) | 18mers_human_m_virus_1MM_329_p.txt | Human papillomavirus type 39, complete genome. |
| 330) | 18mers_human_m_virus_1MM_330_p.txt | Human papillomavirus type 4, complete genome. |
| 331) | 18mers_human_m_virus_1MM_331_p.txt | Human papillomavirus type 40, complete genome. |
| 332) | 18mers_human_m_virus_1MM_332_p.txt | Human papillomavirus type 41, complete genome. |
| 333) | 18mers_human_m_virus_1MM_333_p.txt | Human papillomavirus type 42, complete genome. |
| 334) | 18mers_human_m_virus_1MM_334_p.txt | Human papillomavirus type 44, complete genome. |
| 335) | 18mers_human_m_virus_1MM_335_p.txt | Human papillomavirus type 45, complete genome. |
| 336) | 18mers_human_m_virus_1MM_336_p.txt | Human papillomavirus type 47, complete genome. |
| 337) | 18mers_human_m_virus_1MM_337_p.txt | Human papillomavirus type 48, complete genome. |
| 338) | 18mers_human_m_virus_1MM_338_p.txt | Human papillomavirus type 49, complete genome. |
| 339) | 18mers_human_m_virus_1MM_339_p.txt | Human papillomavirus type 5, complete genome. |
| 340) | 18mers_human_m_virus_1MM_340_p.txt | Human papillomavirus type 50, complete genome. |
| 341) | 18mers_human_m_virus_1MM_341_p.txt | Human papillomavirus type 51, complete genome. |
| 342) | 18mers_human_m_virus_1MM_342_p.txt | Human papillomavirus type 52, complete genome. |
| 343) | 18mers_human_m_virus_1MM_343_p.txt | Human papillomavirus type 53, complete genome. |
| 344) | 18mers_human_m_virus_1MM_344_p.txt | Human papillomavirus type 54, complete genome. |
| 345) | 18mers_human_m_virus_1MM_345_p.txt | Human papillomavirus type 55, complete genome. |
| 346) | 18mers_human_m_virus_1MM_346_p.txt | Human papillomavirus type 56, complete genome. |
| 347) | 18mers_human_m_virus_1MM_347_p.txt | Human papillomavirus type 57, complete genome. |
| 348) | 18mers_human_m_virus_1MM_348_p.txt | Human papillomavirus type 58, complete genome. |
| 349) | 18mers_human_m_virus_1MM_349_p.txt | Human papillomavirus type 59, complete genome. |
| 350) | 18mers_human_m_virus_1MM_350_p.txt | Human papillomavirus type 5b, complete genome. |
| 351) | 18mers_human_m_virus_1MM_351_p.txt | Human papillomavirus type 6, complete genome. |
| 352) | 18mers_human_m_virus_1MM_352_p.txt | Human papillomavirus type 60, complete genome. |
| 353) | 18mers_human_m_virus_1MM_353_p.txt | Human papillomavirus type 61, complete genome. |
| 354) | 18mers_human_m_virus_1MM_354_p.txt | Human papillomavirus type 63, complete genome. |
| 355) | 18mers_human_m_virus_1MM_355_p.txt | Human papillomavirus type 65, complete genome. |
| 356) | 18mers_human_m_virus_1MM_356_p.txt | Human papillomavirus type 66, complete genome. |
| 357) | 18mers_human_m_virus_1MM_357_p.txt | Human papillomavirus type 67, complete genome. |
| 358) | 18mers_human_m_virus_1MM_358_p.txt | Human papillomavirus type 69, complete genome. |
| 359) | 18mers_human_m_virus_1MM_359_p.txt | Human papillomavirus type 6a, complete genome. |
| 360) | 18mers_human_m_virus_1MM_360_p.txt | Human papillomavirus type 6b, complete genome. |
| 361) | 18mers_human_m_virus_1MM_361_p.txt | Human papillomavirus type 7, complete genome. |
| 362) | 18mers_human_m_virus_1MM_362_p.txt | Human papillomavirus type 70, complete genome. |
| 363) | 18mers_human_m_virus_1MM_363_p.txt | Human papillomavirus type 71, complete genome. |
| 364) | 18mers_human_m_virus_1MM_364_p.txt | Human papillomavirus type 74, complete genome. |
| 365) | 18mers_human_m_virus_1MM_365_p.txt | Human papillomavirus type 8, complete genome. |
| 366) | 18mers_human_m_virus_1MM_366_p.txt | Human papillomavirus type 82, complete genome. |
| 367) | 18mers_human_m_virus_1MM_367_p.txt | Human papillomavirus type 83, complete genome. |
| 368) | 18mers_human_m_virus_1MM_368_p.txt | Human papillomavirus type 84, complete genome. |
| 369) | 18mers_human_m_virus_1MM_369_p.txt | Human papillomavirus type 86, complete genome. |
| 370) | 18mers_human_m_virus_1MM_370_p.txt | Human papillomavirus type 87, complete genome. |
| 371) | 18mers_human_m_virus_1MM_371_p.txt | Human papillomavirus type 89, complete genome. |
| 372) | 18mers_human_m_virus_1MM_372_p.txt | Human papillomavirus type 9, complete genome. |
| 373) | 18mers_human_m_virus_1MM_373_p.txt | Human papillomavirus type 90, complete genome. |
| 374) | 18mers_human_m_virus_1MM_374_p.txt | Human papillomavirus type 91, complete genome. |
| 375) | 18mers_human_m_virus_1MM_375_p.txt | Human papillomavirus type 92, complete genome. |
| 376) | 18mers_human_m_virus_1MM_376_p.txt | Monkey B-lymphotropic papovavirus, complete genome. |
| 377) | 18mers_human_m_virus_1MM_377_p.txt | Multimammate rat papillomavirus, complete genome. |
| 378) | 18mers_human_m_virus_1MM_378_p.txt | Ovine papillomavirus 1, complete genome. |
| 379) | 18mers_human_m_virus_1MM_379_p.txt | Ovine papillomavirus 2, complete genome. |
| 380) | 18mers_human_m_virus_1MM_380_p.txt | Phocoena spinipinnis papillomavirus, complete genome. |
| 381) | 18mers_human_m_virus_1MM_381_p.txt | Psittacus erithacus papillomavirus, complete genome. |
| 382) | 18mers_human_m_virus_1MM_382_p.txt | Rabbit oral papillomavirus, complete genome. |
| 383) | 18mers_human_m_virus_1MM_383_p.txt | Reindeer papillomavirus, complete genome. |
| 384) | 18mers_human_m_virus_1MM_384_p.txt | Rhesus monkey papillomavirus, complete genome. |
| 385) | 18mers_human_m_virus_1MM_385_p.txt | Ectocarpus siliculosus virus, complete genome. |
| 386) | 18mers_human_m_virus_1MM_386_p.txt | Paramecium bursaria Chlorella virus 1, complete genome. |
| 387) | 18mers_human_m_virus_1MM_387_p.txt | Acholeplasma phage L2, complete genome. |
| 388) | 18mers_human_m_virus_1MM_388_p.txt | African green monkey polyomavirus, complete genome. |
| 389) | 18mers_human_m_virus_1MM_389_p.txt | BK virus, complete genome. |
| 390) | 18mers_human_m_virus_1MM_390_p.txt | Bovine polyomavirus, complete genome. |
| 391) | 18mers_human_m_virus_1MM_391_p.txt | Budgerigar fledgling polyomavirus, complete genome. |
| 392) | 18mers_human_m_virus_1MM_392_p.txt | JC virus, complete genome. |
| 393) | 18mers_human_m_virus_1MM_393_p.txt | Murine pneumotropic virus, complete genome. |
| 394) | 18mers_human_m_virus_1MM_394_p.txt | Murine polyomavirus strain A2, complete genome. |
| 395) | 18mers_human_m_virus_1MM_395_p.txt | Simian virus 40, complete genome. |
| 396) | 18mers_human_m_virus_1MM_396_p.txt | Amsacta moorei entomopoxvirus, complete genome. |
| 397) | 18mers_human_m_virus_1MM_397_p.txt | Camelpox virus, complete genome. |
| 398) | 18mers_human_m_virus_1MM_398_p.txt | Cowpox virus, complete genome. |
| 399) | 18mers_human_m_virus_1MM_399_p.txt | Ectromelia virus, complete genome. |
| 400) | 18mers_human_m_virus_1MM_400_p.txt | Fowlpox virus, complete genome. |
| 401) | 18mers_human_m_virus_1MM_401_p.txt | Goatpox virus, complete genome. |
| 402) | 18mers_human_m_virus_1MM_402_p.txt | Lumpy skin disease virus, complete genome. |
| 403) | 18mers_human_m_virus_1MM_403_p.txt | Melanoplus sanguinipes entomopoxvirus, complete |
| | | genome. |
| 404) | 18mers_human_m_virus_1MM_404_p.txt | Monkeypox virus, complete genome. |
| 405) | 18mers_human_m_virus_1MM_405_p.txt | Myxoma virus, complete genome. |
| 406) | 18mers_human_m_virus_1MM_406_p.txt | Rabbit fibroma virus, complete genome. |
| 407) | 18mers_human_m_virus_1MM_407_p.txt | Sheeppox virus, complete genome. |
| 408) | 18mers_human_m_virus_1MM_408_p.txt | Swinepox virus, complete genome. |
| 409) | 18mers_human_m_virus_1MM_409_p.txt | Vaccinia virus, complete genome. |
| 410) | 18mers_human_m_virus_1MM_410_p.txt | Variola virus, complete genome. |
| 411) | 18mers_human_m_virus_1MM_411_p.txt | Yaba-like disease virus, complete genome. |
| 412) | 18mers_human_m_virus_1MM_412_p.txt | Sulfolobus virus SIRV-1, complete genome. |
| 413) | 18mers_human_m_virus_1MM_413_p.txt | Sulfolobus virus SIRV-2, complete genome. |
| 414) | 18mers_human_m_virus_1MM_414_p.txt | Enterobacteria phage PRD1, complete genome. |
| 415) | 18mers_human_m_virus_1MM_415_p.txt | Mycoplasma arthritidis bacteriophage MAV1, complete |
| | | genome. |
| 416) | 18mers_human_m_virus_1MM_416_p.txt | Drosophila x virus segment A, complete sequence. |
| 417) | 18mers_human_m_virus_1MM_418_p.txt | Infectious bursal disease virus segment A, complete |
| | | sequence. |
| 418) | 18mers_human_m_virus_1MM_420_p.txt | Infectious pancreatic necrosis virus segment A, complete |
| | | sequence. |
| 419) | 18mers_human_m_virus_1MM_422_p.txt | Marine birnavirus segment A, complete sequence. |
| 420) | 18mers_human_m_virus_1MM_424_p.txt | Bacteriophage phi-12 segment L, complete sequence. |
| 421) | 18mers_human_m_virus_1MM_427_p.txt | Bacteriophage phi-8 segment L, complete sequence. |
| 422) | 18mers_human_m_virus_1MM_430_p.txt | Pseudomonas bacteriophage phi-13 segment L, complete |
| | | sequence. |
| 423) | 18mers_human_m_virus_1MM_433_p.txt | Pseudomonas phage phi-6 segment L, complete sequence. |
| 424) | 18mers_human_m_virus_1MM_436_p.txt | Cryphonectria hypovirus , complete genome. |
| 425) | 18mers_human_m_virus_1MM_437_p.txt | Cryphonectria hypovirus 1, complete genome. |
| 426) | 18mers_human_m_virus_1MM_438_p.txt | Cryphonectria hypovirus 3, complete genome. |
| 427) | 18mers_human_m_virus_1MM_439_p.txt | Atkinsonella hypoxylon partitivirus RNA 1, complete |
| | | sequence. |
| 428) | 18mers_human_m_virus_1MM_442_p.txt | Discula destructiva virus 1 segment 1, complete sequence. |
| 429) | 18mers_human_m_virus_1MM_446_p.txt | Discula destructiva virus 2 segment 1, complete sequence. |
| 430) | 18mers_human_m_virus_1MM_448_p.txt | Fusarium poae virus 1 RNA 1, complete sequence. |
| 431) | 18mers_human_m_virus_1MM_450_p.txt | Gremmeniella abietina RNA virus MSI RNA 1, complete |
| | | sequence. |
| 432) | 18mers_human_m_virus_1MM_453_p.txt | Mycovirus FusoV RNA 1, complete sequence. |
| 433) | 18mers_human_m_virus_1MM_455_p.txt | Rhizoctonia solani virus RNA1, complete sequence. |
| 434) | 18mers_human_m_virus_1MM_457_p.txt | Banna virus segment 1, complete sequence. |
| 435) | 18mers_human_m_virus_1MM_469_p.txt | Bombyx mori cypovirus 1 segment 1, complete sequence. |
| 436) | 18mers_human_m_virus_1MM_479_p.txt | Colorado tick fever virus segment 1, complete sequence. |
| 437) | 18mers_human_m_virus_1MM_491_p.txt | Eyach virus segment 1, complete sequence. |
| 438) | 18mers_human_m_virus_1MM_503_p.txt | Kadipiro virus segment 1, complete sequence. |
| 439) | 18mers_human_m_virus_1MM_515_p.txt | Lymantria dispar cypovirus 1 segment 1, complete |
| | | sequence. |
| 440) | 18mers_human_m_virus_1MM_525_p.txt | Lymantria dispar cypovirus 14 segment 1, complete |
| | | sequence. |
| 441) | 18mers_human_m_virus_1MM_535_p.txt | Mammalian orthoreovirus 1 segment L1, complete |
| | | sequence. |
| 442) | 18mers_human_m_virus_1MM_545_p.txt | Mammalian orthoreovirus 2 segment L1, complete |
| | | sequence. |
| 443) | 18mers_human_m_virus_1MM_555_p.txt | Mammalian orthoreovirus 3 segment L1, complete |
| | | sequence. |
| 444) | 18mers_human_m_virus_1MM_565_p.txt | Nilaparvata lugens reovirus segment 1, complete |
| | | sequence. |
| 445) | 18mers_human_m_virus_1MM_575_p.txt | Rice black streaked dwarf virus segment 1, complete |
| | | sequence. |
| 446) | 18mers_human_m_virus_1MM_585_p.txt | Rice dwarf virus segment 1, complete sequence. |
| 447) | 18mers_human_m_virus_1MM_597_p.txt | Rice ragged stunt virus segment 1, complete sequence. |
| 448) | 18mers_human_m_virus_1MM_607_p.txt | Trichoplusia ni cytoplasmic polyhedrosis virus 15 |
| | | segment 1, complete |
| 449) | 18mers_human_m_virus_1MM_618_p.txt | Eimeria brunetti RNA virus 1, complete genome. |
| 450) | 18mers_human_m_virus_1MM_619_p.txt | Giardia lamblia virus, complete genome. |
| 451) | 18mers_human_m_virus_1MM_620_p.txt | Gremmeniella abietina RNA virus L1, complete genome. |
| 452) | 18mers_human_m_virus_1MM_621_p.txt | Helminthosporium victoriac virus 190S, complete |
| | | genome. |
| 453) | 18mers_human_m_virus_1MM_622_p.txt | Leishmania RNA virus 1-1, complete genome. |
| 454) | 18mers_human_m_virus_1MM_623_p.txt | Leishmania RNA virus 1-4, complete genome. |
| 455) | 18mers_human_m_virus_1MM_624_p.txt | Leishmania RNA virus 2-1, complete genome. |
| 456) | 18mers_human_m_virus_1MM_625_p.txt | Saccharomyces cerevisiae virus L-A, complete genome. |
| 457) | 18mers_human_m_virus_1MM_626_p.txt | Saccharomyces cerevisiae virus L-BC (La), complete |
| | | genome. |
| 458) | 18mers_human_m_virus_1MM_627_p.txt | Sphaeropsis sapinea RNA virus 1, complete genome. |
| 459) | 18mers_human_m_virus_1MM_628_p.txt | Sphaeropsis sapinea RNA virus 2, complete genome. |
| 460) | 18mers_human_m_virus_1MM_629_p.txt | Trichomonas vaginalis virus, complete genome. |
| 461) | 18mers_human_m_virus_1MM_630_p.txt | Trichomonas vaginalis virus 3, complete genome. |
| 462) | 18mers_human_m_virus_1MM_631_p.txt | Trichomonas vaginalis virus II, complete genome. |
| 463) | 18mers_human_m_virus_1MM_632_p.txt | Ustilago maydis virus H1, complete genome. |
| 464) | 18mers_human_m_virus_1MM_633_p.txt | Zygosaccharomyces bailii virus Z, complete genome. |
| 465) | 18mers_human_m_virus_1MM_634_p.txt | Diaporthe ambigua RNA virus 1, complete genome. |
| 466) | 18mers_human_m_virus_1MM_635_p.txt | Beak and feather disease virus, complete genome. |
| 467) | 18mers_human_m_virus_1MM_636_p.txt | Bovine circovirus, complete genome. |
| 468) | 18mers_human_m_virus_1MM_637_p.txt | Canary circovirus, complete genome. |
| 469) | 18mers_human_m_virus_1MM_638_p.txt | Chicken anemia virus, complete genome. |
| 470) | 18mers_human_m_virus_1MM_639_p.txt | Columbid circovirus, complete genome. |
| 471) | 18mers_human_m_virus_1MM_640_p.txt | Goose circovirus, complete genome. |
| 472) | 18mers_human_m_virus_1MM_641_p.txt | Porcine circovirus, complete genome. |
| 473) | 18mers_human_m_virus_1MM_642_p.txt | TT virus, complete genome. |
| 474) | 18mers_human_m_virus_1MM_643_p.txt | TTV-like mini virus, complete genome. |
| 475) | 18mers_human_m_virus_1MM_644_p.txt | Abutilon mosaic virus A component DNA, complete |
| | | sequence. |
| 476) | 18mers_human_m_virus_1MM_646_p.txt | African cassava mosaic virus DNA A, complete sequence. |
| 477) | 18mers_human_m_virus_1MM_648_p.txt | Ageratum enation virus, complete genome. |
| 478) | 18mers_human_m_virus_1MM_649_p.txt | Ageratum yellow vein China virus, complete genome. |
| 479) | 18mers_human_m_virus_1MM_650_p.txt | Ageratum yellow vein Sri Lanka virus segment A, |
| | | complete sequence. |
| 480) | 18mers_human_m_virus_1MM_651_p.txt | Ageratum yellow vein Taiwan virus, complete genome. |
| 481) | 18mers_human_m_virus_1MM_652_p.txt | Ageratum yellow vein virus, complete genome. |
| 482) | 18mers_human_m_virus_1MM_653_p.txt | Bean calico mosaic virus DNA A, complete sequence. |
| 483) | 18mers_human_m_virus_1MM_655_p.txt | Bean dwarf mosaic virus DNA A, complete sequence. |
| 484) | 18mers_human_m_virus_1MM_657_p.txt | Bean golden mosaic virus DNA A, complete sequence. |
| 485) | 18mers_human_m_virus_1MM_659_p.txt | Bean golden yellow mosaic virus DNA A, complete |
| | | sequence. |
| 486) | 18mers_human_m_virus_1MM_661_p.txt | Bean yellow dwarf virus, complete genome. |
| 487) | 18mers_human_m_virus_1MM_662_p.txt | Beet curly top virus, complete genome. |
| 488) | 18mers_human_m_virus_1MM_663_p.txt | Beet mild curly top virus, complete genome. |
| 489) | 18mers_human_m_virus_1MM_664_p.txt | Beet severe curly top virus, complete genome. |
| 490) | 18mers_human_m_virus_1MM_665_p.txt | Bhendi yellow vein mosaic virus, complete genome. |
| 491) | 18mers_human_m_virus_1MM_666_p.txt | Cabbage leaf curl virus DNA A, complete sequence. |
| 492) | 18mers_human_m_virus_1MM_668_p.txt | Chayote yellow mosaic virus, complete genome. |
| 493) | 18mers_human_m_virus_1MM_669_p.txt | Chilli leaf curl virus, complete genome. |
| 494) | 18mers_human_m_virus_1MM_670_p.txt | Chino del tomate virus DNA A, complete sequence. |
| 495) | 18mers_human_m_virus_1MM_672_p.txt | Chloris striate mosaic virus, complete genome. |
| 496) | 18mers_human_m_virus_1MM_673_p.txt | Cotton leaf crumple geminivirus DNA A, complete |
| | | sequence. |
| 497) | 18mers_human_m_virus_1MM_675_p.txt | Cotton leaf curl Alabad virus, complete genome. |
| 498) | 18mers_human_m_virus_1MM_676_p.txt | Cotton leaf curl Gezira virus, complete genome. |
| 499) | 18mers_human_m_virus_1MM_677_p.txt | Cotton leaf curl Kokhran virus, complete genome. |
| 500) | 18mers_human_m_virus_1MM_678_p.txt | Cotton leaf curl Multan virus, complete genome. |
| 501) | 18mers_human_m_virus_1MM_679_p.txt | Cotton leaf curl Rajasthan virus segment A, complete |
| | | sequence. |
| 502) | 18mers_human_m_virus_1MM_680_p.txt | Croton yellow vein mosaic virus, complete genome. |
| 503) | 18mers_human_m_virus_1MM_681_p.txt | Cucurbit leaf curl virus DNA A, complete sequence. |
| 504) | 18mers_human_m_virus_1MM_683_p.txt | Dicliptera yellow mottle virus DNA A, complete |
| | | sequence. |
| 505) | 18mers_human_m_virus_1MM_685_p.txt | Digitaria streak virus, complete genome. |
| 506) | 18mers_human_m_virus_1MM_686_p.txt | East African cassava mosaic Cameroon virus DNA A, |
| | | complete sequence. |
| 507) | 18mers_human_m_virus_1MM_688_p.txt | East African cassava mosaic virus DNA A, complete |
| | | sequence. |
| 508) | 18mers_human_m_virus_1MM_690_p.txt | East African cassava mosaic Zanzibar virus DNA A, |
| | | complete sequence. |
| 509) | 18mers_human_m_virus_1MM_692_p.txt | Egyptian sugarcane streak virus, complete genome. |
| 510) | 18mers_human_m_virus_1MM_693_p.txt | Eupatorium yellow vein virus, complete genome. |
| 511) | 18mers_human_m_virus_1MM_694_p.txt | Hollyhock leaf crumple virus, complete genome. |
| 512) | 18mers_human_m_virus_1MM_695_p.txt | Honeysuckle yellow vein mosaic virus, complete genome. |
| 513) | 18mers_human_m_virus_1MM_696_p.txt | Horseradish curly top virus, complete genome. |
| 514) | 18mers_human_m_virus_1MM_697_p.txt | Indian cassava mosaic virus DNA B, complete sequence. |
| 515) | 18mers_human_m_virus_1MM_699_p.txt | Ipomoea yellow vein virus, complete genome. |
| 516) | 18mers_human_m_virus_1MM_700_p.txt | Macroptilium mosaic Puerto Rico virus DNA A, complete |
| | | sequence. |
| 517) | 18mers_human_m_virus_1MM_702_p.txt | Macroptilium yellow mosaic Florida virus DNA A, |
| | | complete sequence. |
| 518) | 18mers_human_m_virus_1MM_704_p.txt | Macroptilium yellow mosaic virus, complete genome. |
| 519) | 18mers_human_m_virus_1MM_705_p.txt | Maize streak virus, complete genome. |
| 520) | 18mers_human_m_virus_1MM_706_p.txt | Malvastrum yellow vein virus, complete genome. |
| 521) | 18mers_human_m_virus_1MM_707_p.txt | Melon chlorotic leaf curl virus, complete genome. |
| 522) | 18mers_human_m_virus_1MM_708_p.txt | Miscanthus streak virus, complete genome. |
| 523) | 18mers_human_m_virus_1MM_709_p.txt | Mungbean yellow mosaic India virus DNA A, complete |
| | | sequence. |
| 524) | 18mers_human_m_virus_1MM_711_p.txt | Mungbean yellow mosaic virus DNA A, complete |
| | | sequence. |
| 525) | 18mers_human_m_virus_1MM_713_p.txt | Okra yellow vein mosaic virus, complete genome. |
| 526) | 18mers_human_m_virus_1MM_714_p.txt | Panicum streak virus, complete genome. |
| 527) | 18mers_human_m_virus_1MM_715_p.txt | Papaya leaf curl virus, complete genome. |
| 528) | 18mers_human_m_virus_1MM_716_p.txt | Pepper golden mosaic virus DNA A, complete sequence. |
| 529) | 18mers_human_m_virus_1MM_718_p.txt | Pepper huasteco yellow vein virus DNA A, complete |
| | | sequence. |
| 530) | 18mers_human_m_virus_1MM_720_p.txt | Pepper leaf curl Bangladesh virus segment A component, |
| | | complete sequence. |
| 531) | 18mers_human_m_virus_1MM_721_p.txt | Pepper leaf curl virus DNA A, complete sequence. |
| 532) | 18mers_human_m_virus_1MM_722_p.txt | Potato yellow mosaic Panama virus DNA A, complete |
| | | sequence. |
| 533) | 18mers_human_m_virus_1MM_724_p.txt | Potato yellow mosaic Trinidad virus DNA A, complete |
| | | sequence. |
| 534) | 18mers_human_m_virus_1MM_726_p.txt | Potato yellow mosaic virus DNA A, complete sequence. |
| 535) | 18mers_human_m_virus_1MM_728_p.txt | Rhynchosia Golden Mosaic Virus chromosome DNA A, |
| | | complete sequence. |
| 536) | 18mers_human_m_virus_1MM_729_p.txt | Sida golden mosaic Costa Rica virus DNA A, complete |
| | | sequence. |
| 537) | 18mers_human_m_virus_1MM_731_p.txt | Sida golden mosaic Florida virus, complete genome. |
| 538) | 18mers_human_m_virus_1MM_732_p.txt | Sida golden mosaic Honduras virus DNA A, complete |
| | | sequence. |
| 539) | 18mers_human_m_virus_1MM_734_p.txt | Sida golden mosaic virus DNA-A, complete sequence. |
| 540) | 18mers_human_m_virus_1MM_736_p.txt | Sida golden yellow vein virus, complete genome. |
| 541) | 18mers_human_m_virus_1MM_737_p.txt | Sida mottle virus, complete genome. |
| 542) | 18mers_human_m_virus_1MM_738_p.txt | Sida yellow mosaic virus, complete genome. |
| 543) | 18mers_human_m_virus_1MM_739_p.txt | Sida yellow vein virus DNA A, complete sequence. |
| 544) | 18mers_human_m_virus_1MM_741_p.txt | South African cassava mosaic virus DNA A, complete |
| | | sequence. |
| 545) | 18mers_human_m_virus_1MM_743_p.txt | Soybean crinkle leaf virus, complete genome. |
| 546) | 18mers_human_m_virus_1MM_744_p.txt | Squash leaf curl China virus, complete genome. |
| 547) | 18mers_human_m_virus_1MM_745_p.txt | Squash leaf curl virus A component DNA, complete |
| | | sequence. |
| 548) | 18mers_human_m_virus_1MM_747_p.txt | Squash leaf curl Yunnan virus, complete genome. |
| 549) | 18mers_human_m_virus_1MM_748_p.txt | Squash mild leaf curl virus DNA A, complete sequence. |
| 550) | 18mers_human_m_virus_1MM_750_p.txt | Squash yellow mild mottle virus DNA A, complete |
| | | sequence. |
| 551) | 18mers_human_m_virus_1MM_752_p.txt | Sri Lankan cassava mosaic virus DNA A, complete |
| | | sequence. |
| 552) | 18mers_human_m_virus_1MM_754_p.txt | Stachytarpheta leaf curl virus, complete genome. |
| 553) | 18mers_human_m_virus_1MM_755_p.txt | Sugarcane streak Reunion virus, complete genome. |
| 554) | 18mers_human_m_virus_1MM_756_p.txt | Sugarcane streak virus, complete genome. |
| 555) | 18mers_human_m_virus_1MM_757_p.txt | Sweet potato leaf curl Georgia virus, complete genome. |
| 556) | 18mers_human_m_virus_1MM_758_p.txt | Sweet potato leaf curl virus, complete genome. |
| 557) | 18mers_human_m_virus_1MM_759_p.txt | Tobacco curly shoot virus, complete genome. |
| 558) | 18mers_human_m_virus_1MM_760_p.txt | Tobacco leaf curl Japan virus, complete genome. |
| 559) | 18mers_human_m_virus_1MM_761_p.txt | Tobacco leaf curl Kochi virus, complete genome. |
| 560) | 18mers_human_m_virus_1MM_762_p.txt | Tobacco leaf curl Yamaguchi virus, complete genome. |
| 561) | 18mers_human_m_virus_1MM_763_p.txt | Tobacco leaf curl Yunnan virus, complete genome. |
| 562) | 18mers_human_m_virus_1MM_764_p.txt | Tobacco leaf curl Zimbabwe virus, complete genome. |
| 563) | 18mers_human_m_virus_1MM_765_p.txt | Tobacco yellow dwarf virus, complete genome. |
| 564) | 18mers_human_m_virus_1MM_766_p.txt | Tomato chlorotic mottle virus DNA A, complete |
| | | sequence. |
| 565) | 18mers_human_m_virus_1MM_768_p.txt | Tomato curly stunt virus, complete genome. |
| 566) | 18mers_human_m_virus_1MM_769_p.txt | Tomato golden mosaic virus DNA A, complete sequence. |
| 567) | 18mers_human_m_virus_1MM_771_p.txt | Tomato golden mottle virus, complete genome. |
| 568) | 18mers_human_m_virus_1MM_772_p.txt | Tomato leaf curl Bangalore virus, complete genome. |
| 569) | 18mers_human_m_virus_1MM_773_p.txt | Tomato leaf curl Bangladesh virus, complete genome. |
| 570) | 18mers_human_m_virus_1MM_774_p.txt | Tomato leaf curl Gujarat virus DNA A, complete |
| | | sequence. |
| 571) | 18mers_human_m_virus_1MM_776_p.txt | Tomato leaf curl Karnataka virus, complete genome. |
| 572) | 18mers_human_m_virus_1MM_777_p.txt | Tomato leaf curl Laos virus, complete genome. |
| 573) | 18mers_human_m_virus_1MM_778_p.txt | Tomato leaf curl Malaysia virus, complete genome. |
| 574) | 18mers_human_m_virus_1MM_779_p.txt | Tomato leaf curl New Delhi virus DNA A, complete |
| | | sequence. |
| 575) | 18mers_human_m_virus_1MM_781_p.txt | Tomato leaf curl Sri Lanka virus, complete genome. |
| 576) | 18mers_human_m_virus_1MM_782_p.txt | Tomato leaf curl Taiwan virus, complete genome. |
| 577) | 18mers_human_m_virus_1MM_783_p.txt | Tomato leaf curl Vietnam virus, complete genome. |
| 578) | 18mers_human_m_virus_1MM_784_p.txt | Tomato leaf curl virus, complete genome. |
| 579) | 18mers_human_m_virus_1MM_785_p.txt | Tomato mosaic Havana virus DNA A, complete sequence. |
| 580) | 18mers_human_m_virus_1MM_787_p.txt | Tomato mottle Taino virus DNA A, complete sequence. |
| 581) | 18mers_human_m_virus_1MM_789_p.txt | Tomato mottle virus DNA A, complete sequence. |
| 582) | 18mers_human_m_virus_1MM_791_p.txt | Tomato pseudo-curly top virus, complete genome. |
| 583) | 18mers_human_m_virus_1MM_792_p.txt | Tomato rugose mosaic virus DNA A, complete sequence. |
| 584) | 18mers_human_m_virus_1MM_794_p.txt | Tomato severe leaf curl virus, complete genome. |
| 585) | 18mers_human_m_virus_1MM_795_p.txt | Tomato yellow leaf curl China virus, complete genome. |
| 586) | 18mers_human_m_virus_1MM_796_p.txt | Tomato yellow leaf curl Gezira virus, complete genome. |
| 587) | 18mers_human_m_virus_1MM_797_p.txt | Tomato yellow leaf curl Malaga virus, complete genome. |
| 588) | 18mers_human_m_virus_1MM_798_p.txt | Tomato yellow leaf curl Sardinia virus, complete genome. |
| 589) | 18mers_human_m_virus_1MM_799_p.txt | Tomato yellow leaf curl Thailand virus DNA A, complete |
| | | sequence. |
| 590) | 18mers_human_m_virus_1MM_801_p.txt | Tomato yellow leaf curl virus, complete genome. |
| 591) | 18mers_human_m_virus_1MM_802_p.txt | Watermelon chlorotic stunt virus DNA A, complete |
| | | sequence. |
| 592) | 18mers_human_m_virus_1MM_804_p.txt | Acholeplasma phage MV-L1, complete genome. |
| 593) | 18mers_human_m_virus_1MM_805_p.txt | Bacteriophage VfO3K6, complete genome. |
| 594) | 18mers_human_m_virus_1MM_806_p.txt | Bacteriophage VfO4K68, complete genome. |
| 595) | 18mers_human_m_virus_1MM_807_p.txt | Bacteriophage VSKK, complete genome. |
| 596) | 18mers_human_m_virus_1MM_808_p.txt | Coliphage M13, complete genome. |
| 597) | 18mers_human_m_virus_1MM_809_p.txt | Enterobacteria phage l2-2. |
| 598) | 18mers_human_m_virus_1MM_810_p.txt | Enterobacteria phage lf1, complete genome. |
| 599) | 18mers_human_m_virus_1MM_811_p.txt | Enterobacteria phage lke. |
| 600) | 18mers_human_m_virus_1MM_812_p.txt | Propionibacterium phage phiB5, complete genome. |
| 601) | 18mers_human_m_virus_1MM_813_p.txt | Pseudomonas phage Pf1, complete genome. |
| 602) | 18mers_human_m_virus_1MM_814_p.txt | Pseudomonas phage Pf3, complete genome. |
| 603) | 18mers_human_m_virus_1MM_815_p.txt | Spiroplasma phage 1-C74, complete genome. |
| 604) | 18mers_human_m_virus_1MM_816_p.txt | Spiroplasma phage 1-R8A2B, complete genome. |
| 605) | 18mers_human_m_virus_1MM_817_p.txt | SVTS2 plectrovirus, complete genome. |
| 606) | 18mers_human_m_virus_1MM_818_p.txt | Vibrio cholerae filamentous bacteriophage fs-2, complete |
| | | genome. |
| 607) | 18mers_human_m_virus_1MM_819_p.txt | Vibrio cholerae O139 fs1 phage, complete genome. |
| 608) | 18mers_human_m_virus_1MM_820_p.txt | Vibrio cholerae phage VGJphi virion, complete genome. |
| 609) | 18mers_human_m_virus_1MM_821_p.txt | Vibrio phage VSK, complete genome. |
| 610) | 18mers_human_m_virus_1MM_822_p.txt | Xanthomonas phage Cf1c, complete genome. |
| 611) | 18mers_human_m_virus_1MM_823_p.txt | Chlamydia phage 2 virion, complete genome. |
| 612) | 18mers_human_m_virus_1MM_824_p.txt | Chlamydia phage phiCPAR39, complete genome. |
| 613) | 18mers_human_m_virus_1MM_825_p.txt | Chlamydia phage PhiCPG1, complete genome. |
| 614) | 18mers_human_m_virus_1MM_826_p.txt | Chlamydia psittaci bacteriophage chp1, complete genome. |
| 615) | 18mers_human_m_virus_1MM_827_p.txt | Coliphage alpha3, complete genome. |
| 616) | 18mers_human_m_virus_1MM_828_p.txt | Coliphage phiX174, complete genome. |
| 617) | 18mers_human_m_virus_1MM_829_p.txt | Enterobacteria phage G4, complete genome. |
| 618) | 18mers_human_m_virus_1MM_830_p.txt | Enterobacteria phage S13, complete genome. |
| 619) | 18mers_human_m_virus_1MM_831_p.txt | Phage phiMH2K virion, complete genome. |
| 620) | 18mers_human_m_virus_1MM_832_p.txt | Spiroplasma phage 4, complete genome. |
| 621) | 18mers_human_m_virus_1MM_833_p.txt | Ageratum yellow vein virus-associated nanovirus DNA, |
| | | complete genome. |
| 622) | 18mers_human_m_virus_1MM_834_p.txt | Banana bunchy top virus DNA 1, complete sequence. |
| 623) | 18mers_human_m_virus_1MM_840_p.txt | Coconut foliar decay virus, complete genome. |
| 624) | 18mers_human_m_virus_1MM_841_p.txt | Faba bean necrotic yellows virus DNA 1, complete |
| | | sequence. |
| 625) | 18mers_human_m_virus_1MM_851_p.txt | Milk vetch dwarf virus segment 1, complete sequence. |
| 626) | 18mers_human_m_virus_1MM_862_p.txt | Subterranean clover stunt virus DNA 1, complete |
| | | sequence. |
| 627) | 18mers_human_m_virus_1MM_870_p.txt | Acheta domesticus densovirus, complete genome. |
| 628) | 18mers_human_m_virus_1MM_871_p.txt | Adeno-associated virus 1, complete genome. |
| 629) | 18mers_human_m_virus_1MM_872_p.txt | Adeno-associated virus 2, complete genome. |
| 630) | 18mers_human_m_virus_1MM_873_p.txt | Adeno-associated virus 3, complete genome. |
| 631) | 18mers_human_m_virus_1MM_874_p.txt | Adeno-associated virus 3B, complete genome. |
| 632) | 18mers_human_m_virus_1MM_875_p.txt | Adeno-associated virus 4, complete genome. |
| 633) | 18mers_human_m_virus_1MM_876_p.txt | Adeno-associated virus 6, complete genome. |
| 634) | 18mers_human_m_virus_1MM_877_p.txt | Aedes albopictus densovirus, complete genome. |
| 635) | 18mers_human_m_virus_1MM_878_p.txt | Aleutian mink disease parvovirus, complete genome. |
| 636) | 18mers_human_m_virus_1MM_879_p.txt | B19 virus, complete genome. |
| 637) | 18mers_human_m_virus_1MM_880_p.txt | Bombyx mori densovirus 1, complete genome. |
| 638) | 18mers_human_m_virus_1MM_881_p.txt | Bombyx mori densovirus 5, complete genome. |
| 639) | 18mers_human_m_virus_1MM_882_p.txt | Bovine parvovirus, complete genome. |
| 640) | 18mers_human_m_virus_1MM_883_p.txt | Canine parvovirus, complete genome. |
| 641) | 18mers_human_m_virus_1MM_884_p.txt | Casphalia extranea densovirus, complete genome. |
| 642) | 18mers_human_m_virus_1MM_885_p.txt | Diatraea saccharalis densovirus, complete genome. |
| 643) | 18mers_human_m_virus_1MM_886_p.txt | Galleria mellonella densovirus, complete genome. |
| 644) | 18mers_human_m_virus_1MM_887_p.txt | Goose parvovirus, complete genome. |
| 645) | 18mers_human_m_virus_1MM_888_p.txt | Human erythrovirus V9, complete genome. |
| 646) | 18mers_human_m_virus_1MM_889_p.txt | Infectious hypodermal and hematopoietic necrosis virus, |
| | | complete genome. |
| 647) | 18mers_human_m_virus_1MM_890_p.txt | Junonia coenia densovirus, complete genome. |
| 648) | 18mers_human_m_virus_1MM_891_p.txt | LullI virus, complete genome. |
| 649) | 18mers_human_m_virus_1MM_892_p.txt | Mice minute virus, complete genome. |
| 650) | 18mers_human_m_virus_1MM_893_p.txt | Minute virus of canines, genome. |
| 651) | 18mers_human_m_virus_1MM_894_p.txt | Mouse parvovirus I, complete genome. |
| 652) | 18mers_human_m_virus_1MM_895_p.txt | Parvovirus H1, complete genome. |
| 653) | 18mers_human_m_virus_1MM_896_p.txt | Periplaneta fuliginosa densovirus, complete genome. |
| 654) | 18mers_human_m_virus_1MM_897_p.txt | Planococcus citri densovirus, complete genome. |
| 655) | 18mers_human_m_virus_1MM_898_p.txt | Porcine parvovirus, complete genome. |
| 656) | 18mers_human_m_virus_1MM_899_p.txt | Lassa virus segment L, complete sequence. |
| 657) | 18mers_human_m_virus_1MM_901_p.txt | Lymphocytic choriomeningitis virus segment L, complete |
| | | sequence. |
| 658) | 18mers_human_m_virus_1MM_903_p.txt | Tacaribe virus segment L, complete sequence. |
| 659) | 18mers_human_m_virus_1MM_905_p.txt | Andes virus segment L, complete sequence. |
| 660) | 18mers_human_m_virus_1MM_908_p.txt | Bunyamwera virus L segment, complete sequence. |
| 661) | 18mers_human_m_virus_1MM_911_p.txt | Dugbe virus segment L, complete sequence. |
| 662) | 18mers_human_m_virus_1MM_914_p.txt | Groundnut bud necrosis virus segment L, complete |
| | | sequence. |
| 663) | 18mers_human_m_virus_1MM_917_p.txt | Impatiens necrotic spot virus segment L, complete |
| | | sequence. |
| 664) | 18mers_human_m_virus_1MM_920_p.txt | La Crosse virus segment L, complete sequence. |
| 665) | 18mers_human_m_virus_1MM_923_p.txt | Rift Valley fever virus, complete genome. |
| 666) | 18mers_human_m_virus_1MM_926_p.txt | Tomato spotted wilt virus RNA L, complete sequence. |
| 667) | 18mers_human_m_virus_1MM_929_p.txt | Watermelon spotted wilt virus segment L, complete |
| | | sequence. |
| 668) | 18mers_human_m_virus_1MM_932_p.txt | Australian bat lyssavirus, complete genome. |
| 669) | 18mers_human_m_virus_1MM_933_p.txt | Avian paramyxovirus 6, complete genome. |
| 670) | 18mers_human_m_virus_1MM_934_p.txt | Boma disease virus, complete genome. |
| 671) | 18mers_human_m_virus_1MM_935_p.txt | Bovine ephemeral fever virus, complete genome. |
| 672) | 18mers_human_m_virus_1MM_936_p.txt | Bovine parainfluenza virus 3, complete genome. |
| 673) | 18mers_human_m_virus_1MM_937_p.txt | Canine distemper virus, complete genome. |
| 674) | 18mers_human_m_virus_1MM_938_p.txt | Hendra virus, complete genome. |
| 675) | 18mers_human_m_virus_1MM_939_p.txt | Human metapneumovirus, genome. |
| 676) | 18mers_human_m_virus_1MM_940_p.txt | Human parainfluenza virus 1 strain Washington/1964, |
| | | complete genome. |
| 677) | 18mers_human_m_virus_1MM_941_p.txt | Human parainfluenza virus 2, complete genome. |
| 678) | 18mers_human_m_virus_1MM_942_p.txt | Human parainfluenza virus 3, complete genome. |
| 679) | 18mers_human_m_virus_1MM_943_p.txt | Human respiratory syncytial virus, complete genome. |
| 680) | 18mers_human_m_virus_1MM_944_p.txt | Infectious hematopoietic necrosis virus, complete |
| | | genome. |
| 681) | 18mers_human_m_virus_1MM_945_p.txt | Marburg virus, complete genome. |
| 682) | 18mers_human_m_virus_1MM_946_p.txt | Measles virus, complete genome. |
| 683) | 18mers_human_m_virus_1MM_947_p.txt | Mumps virus, complete genome. |
| 684) | 18mers_human_m_virus_1MM_948_p.txt | Newcastle disease virus, complete genome. |
| 685) | 18mers_human_m_virus_1MM_949_p.txt | Nipah virus, complete genome. |
| 686) | 18mers_human_m_virus_1MM_950_p.txt | Northern cereal mosaic virus, complete genome. |
| 687) | 18mers_human_m_virus_1MM_951_p.txt | Rabies virus, complete genome. |
| 688) | 18mers_human_m_virus_1MM_952_p.txt | Respiratory syncytial virus, complete genome. |
| 689) | 18mers_human_m_virus_1MM_953_p.txt | Reston Ebola virus, complete genome. |
| 690) | 18mers_human_m_virus_1MM_954_p.txt | Rice yellow stunt virus, complete genome. |
| 691) | 18mers_human_m_virus_1MM_955_p.txt | Sendai virus, complete genome. |
| 692) | 18mers_human_m_virus_1MM_956_p.txt | Snakehead rhabdovirus, complete genome. |
| 693) | 18mers_human_m_virus_1MM_957_p.txt | Sonchus yellow net virus, complete genome. |
| 694) | 18mers_human_m_virus_1MM_958_p.txt | Spring viremia of carp virus, complete genome. |
| 695) | 18mers_human_m_virus_1MM_959_p.txt | Tioman virus, complete genome. |
| 696) | 18mers_human_m_virus_1MM_960_p.txt | Tupaia paramyxovirus, complete genome. |
| 697) | 18mers_human_m_virus_1MM_961_p.txt | Vesicular stomatitis Indiana virus, complete genome. |
| 698) | 18mers_human_m_virus_1MM_962_p.txt | Viral hemorrhagic septicemia virus, complete genome. |
| 699) | 18mers_human_m_virus_1MM_963_p.txt | Zaire Ebola virus, complete genome. |
| 700) | 18mers_human_m_virus_1MM_964_p.txt | Mirafiori lettuce virus RNA 1, complete sequence. |
| 701) | 18mers_human_m_virus_1MM_968_p.txt | Influenza A virus RNA segment 1, complete sequence. |
| 702) | 18mers_human_m_virus_1MM_976_p.txt | Influenza A virus (A/Puerto Rico/8/34/Mount |
| | | Sinai(H1N1)) segment 1, complete |
| 703) | 18mers_human_m_virus_1MM_984_p.txt | Influenza B virus RNA-1, complete sequence. |
| 704) | 18mers_human_m_virus_1MM_992_p.txt | Influenza B virus (B/Memphis/12/97) segment 1, |
| | | complete sequence. |
| 705) | 18mers_human_m_virus_1MM_1000_p.txt | Influenza B virus (B/Memphis/12/97-MA) segment 1, |
| | | complete sequence. |
| 706) | 18mers_human_m_virus_1MM_1008_p.txt | Rice grassy stunt virus RNA 1, complete sequence. |
| 707) | 18mers_human_m_virus_1MM_1014_p.txt | Rice stripe virus RNA 1, complete sequence. |
| 708) | 18mers_human_m_virus_1MM_1018_p.txt | Garlic virus C, complete genome. |
| 709) | 18mers_human_m_virus_1MM_1019_p.txt | Garlic virus E, complete genome. |
| 710) | 18mers_human_m_virus_1MM_1020_p.txt | Garlic virus X, complete genome. |
| 711) | 18mers_human_m_virus_1MM_1021_p.txt | Shallot virus X, complete genome. |
| 712) | 18mers_human_m_virus_1MM_1022_p.txt | Avian nephritis virus, complete genome. |
| 713) | 18mers_human_m_virus_1MM_1023_p.txt | Human astrovirus, complete genome. |
| 714) | 18mers_human_m_virus_1MM_1024_p.txt | Mink astrovirus, complete genome. |
| 715) | 18mers_human_m_virus_1MM_1025_p.txt | Ovine astrovirus, complete genome. |
| 716) | 18mers_human_m_virus_1MM_1026_p.txt | Turkey astrovirus, complete genome. |
| 717) | 18mers_human_m_virus_1MM_1027_p.txt | Mushroom bacilliform virus, complete genome. |
| 718) | 18mers_human_m_virus_1MM_1028_p.txt | Beet necrotic yellow vein virus RNA 1, complete |
| | | sequence. |
| 719) | 18mers_human_m_virus_1MM_1033_p.txt | Beet soil-borne mosaic virus RNA1, complete sequence. |
| 720) | 18mers_human_m_virus_1MM_1037_p.txt | Alfalfa mosaic virus RNA 1, complete sequence. |
| 721) | 18mers_human_m_virus_1MM_1040_p.txt | American plum line pattern virus RNA1, complete |
| | | sequence. |
| 722) | 18mers_human_m_virus_1MM_1043_p.txt | Apple mosaic virus RNA 1, complete sequence. |
| 723) | 18mers_human_m_virus_1MM_1046_p.txt | Broad bean mottle virus RNA 1, complete sequence. |
| 724) | 18mers_human_m_virus_1MM_1049_p.txt | Brome mosaic virus RNA 1, complete sequence. |
| 725) | 18mers_human_m_virus_1MM_1052_p.txt | Citrus leaf rugose virus RNA 1, complete sequence. |
| 726) | 18mers_human_m_virus_1MM_1055_p.txt | Cowpea chlorotic mottle virus RNA 1, complete |
| | | sequence. |
| 727) | 18mers_human_m_virus_1MM_1058_p.txt | Cucumber mosaic virus RNA 1, complete sequence. |
| 728) | 18mers_human_m_virus_1MM_1061_p.txt | Elm mottle virus RNA 1, complete sequence. |
| 729) | 18mers_human_m_virus_1MM_1064_p.txt | Olive latent virus 2 RNA 1, complete sequence. |
| 730) | 18mers_human_m_virus_1MM_1067_p.txt | Peanut stunt virus RNA 1, complete sequence. |
| 731) | 18mers_human_m_virus_1MM_1070_p.txt | Pelargonium zonate spot virus RNA 1, complete |
| | | sequence. |
| 732) | 18mers_human_m_virus_1MM_1073_p.txt | Prunus necrotic ringspot virus RNA1, complete sequence. |
| 733) | 18mers_human_m_virus_1MM_1076_p.txt | Spinach latent virus RNA 1, complete sequence. |
| 734) | 18mers_human_m_virus_1MM_1079_p.txt | Spring beauty latent virus RNA 1, complete sequence. |
| 735) | 18mers_human_m_virus_1MM_1082_p.txt | Tobacco streak virus RNA 1, complete sequence. |
| 736) | 18mers_human_m_virus_1MM_1085_p.txt | Tomato aspermy virus RNA 1, complete sequence. |
| 737) | 18mers_human_m_virus_1MM_1088_p.txt | Tulare apple mosaic virus RNA1, complete sequence. |
| 738) | 18mers_human_m_virus_1MM_1091_p.txt | Calicivirus strain NB, complete genome. |
| 739) | 18mers_human_m_virus_1MM_1092_p.txt | Canine calicivirus, complete genome. |
| 740) | 18mers_human_m_virus_1MM_1093_p.txt | European brown hare syndrome virus, complete genome. |
| 741) | 18mers_human_m_virus_1MM_1094_p.txt | Feline calicivirus, complete genome. |
| 742) | 18mers_human_m_virus_1MM_1095_p.txt | Norwalk virus, complete genome. |
| 743) | 18mers_human_m_virus_1MM_1096_p.txt | Porcine enteric calicivirus, complete genome. |
| 744) | 18mers_human_m_virus_1MM_1097_p.txt | Rabbit hemorrhagic disease virus, complete genome. |
| 745) | 18mers_human_m_virus_1MM_1098_p.txt | Vesicular exanthema of swine virus, complete genome. |
| 746) | 18mers_human_m_virus_1MM_1099_p.txt | Walrus calicivirus, complete genome. |
| 747) | 18mers_human_m_virus_1MM_1100_p.txt | Apple stem grooving virus, complete genome. |
| 748) | 18mers_human_m_virus_1MM_1101_p.txt | Cherry virus A, complete genome. |
| 749) | 18mers_human_m_virus_1MM_1102_p.txt | Aconitum latent virus, complete genome. |
| 750) | 18mers_human_m_virus_1MM_1103_p.txt | Blueberry scorch virus, complete genome. |
| 751) | 18mers_human_m_virus_1MM_1104_p.txt | Garlic latent virus, complete genome. |
| 752) | 18mers_human_m_virus_1MM_1105_p.txt | Hop latent virus, complete genome. |
| 753) | 18mers_human_m_virus_1MM_1106_p.txt | Potato virus M, complete genome. |
| 754) | 18mers_human_m_virus_1MM_1107_p.txt | Beet yellows virus, complete genome. |
| 755) | 18mers_human_m_virus_1MM_1108_p.txt | Citrus tristeza virus, complete genome. |
| 756) | 18mers_human_m_virus_1MM_1109_p.txt | Cucumber yellows virus RNA 1, complete sequence. |
| 757) | 18mers_human_m_virus_1MM_1111_p.txt | Grapevine leafroll-associated virus 3, complete genome. |
| 758) | 18mers_human_m_virus_1MM_1112_p.txt | Grapevine rootstock stem lesion associated virus, |
| | | complete genome. |
| 759) | 18mers_human_m_virus_1MM_1113_p.txt | Lettuce infectious yellows virus RNA 1, complete |
| | | sequence. |
| 760) | 18mers_human_m_virus_1MM_1115_p.txt | Little cherry virus 1, complete genome. |
| 761) | 18mers_human_m_virus_1MM_1116_p.txt | Sweet potato chlorotic stunt virus RNA 1, complete |
| | | sequence. |
| 762) | 18mers_human_m_virus_1MM_1118_p.txt | Bean pod mottle virus RNA1, complete sequence. |
| 763) | 18mers_human_m_virus_1MM_1120_p.txt | Beet ringspot virus RNA 1, complete sequence. |
| 764) | 18mers_human_m_virus_1MM_1122_p.txt | Blackcurrant reversion virus RNA1, complete sequence. |
| 765) | 18mers_human_m_virus_1MM_1124_p.txt | Broad bean wilt virus 2 RNA1, complete sequence. |
| 766) | 18mers_human_m_virus_1MM_1126_p.txt | Cowpea mosaic virus RNA 1, complete sequence. |
| 767) | 18mers_human_m_virus_1MM_1128_p.txt | Cowpea severe mosaic virus RNA 1, complete sequence. |
| 768) | 18mers_human_m_virus_1MM_1130_p.txt | Cycas necrotic stunt virus RNA 1, complete sequence. |
| 769) | 18mers_human_m_virus_1MM_1132_p.txt | Grapevine chrome mosaic virus RNA 1, complete |
| | | sequence. |
| 770) | 18mers_human_m_virus_1MM_1134_p.txt | Grapevine fanleaf virus RNA 1, complete sequence. |
| 771) | 18mers_human_m_virus_1MM_1136_p.txt | Patchouli mild mosaic virus RNA 1, complete sequence. |
| 772) | 18mers_human_m_virus_1MM_1138_p.txt | Red clover mottle virus RNA 1, complete sequence. |
| 773) | 18mers_human_m_virus_1MM_1140_p.txt | Squash mosaic virus RNA 1, complete sequence. |
| 774) | 18mers_human_m_virus_1MM_1142_p.txt | Tomato black ring virus RNA 1, complete sequence. |
| 775) | 18mers_human_m_virus_1MM_1144_p.txt | Tomato ringspot virus RNA 1, complete sequence. |
| 776) | 18mers_human_m_virus_1MM_1146_p.txt | Acute bee paralysis virus, complete genome. |
| 777) | 18mers_human_m_virus_1MM_1147_p.txt | Aphid lethal paralysis virus, complete genome. |
| 778) | 18mers_human_m_virus_1MM_1148_p.txt | Black queen cell virus, complete genome. |
| 779) | 18mers_human_m_virus_1MM_1149_p.txt | Cricket paralysis virus, complete genome. |
| 780) | 18mers_human_m_virus_1MM_1150_p.txt | Drosophila C virus, complete genome. |
| 781) | 18mers_human_m_virus_1MM_1151_p.txt | Himetobi P virus, complete genome. |
| 782) | 18mers_human_m_virus_1MM_1152_p.txt | Plautia stali intestine virus, complete genome. |
| 783) | 18mers_human_m_virus_1MM_1153_p.txt | Rhopalosiphum padi virus, complete genome. |
| 784) | 18mers_human_m_virus_1MM_1154_p.txt | Taura syndrome virus, complete genome. |
| 785) | 18mers_human_m_virus_1MM_1155_p.txt | Triatoma virus, complete genome. |
| 786) | 18mers_human_m_virus_1MM_1156_p.txt | Alkhurma virus, complete genome. |
| 787) | 18mers_human_m_virus_1MM_1157_p.txt | Apoi virus, genome. |
| 788) | 18mers_human_m_virus_1MM_1158_p.txt | Bovine viral diarrhea virus genotype 2, complete genome. |
| 789) | 18mers_human_m_virus_1MM_1159_p.txt | Cell fusing agent virus, complete genome. |
| 790) | 18mers_human_m_virus_1MM_1160_p.txt | Deer tick virus, genome. |
| 791) | 18mers_human_m_virus_1MM_1161_p.txt | Dengue virus, complete genome. |
| 792) | 18mers_human_m_virus_1MM_1162_p.txt | Hepatitis C virus, complete genome. |
| 793) | 18mers_human_m_virus_1MM_1163_p.txt | Hepatitis G virus, complete genome. |
| 794) | 18mers_human_m_virus_1MM_1164_p.txt | Hepatitis GB virus A, complete genome. |
| 795) | 18mers_human_m_virus_1MM_1165_p.txt | Hepatitis GB virus B, complete genome. |
| 796) | 18mers_human_m_virus_1MM_1166_p.txt | Hepatitis GB virus C, complete genome. |
| 797) | 18mers_human_m_virus_1MM_1167_p.txt | Japanese encephalitis virus, genome. |
| 798) | 18mers_human_m_virus_1MM_1168_p.txt | Langat virus, complete genome. |
| 799) | 18mers_human_m_virus_1MM_1169_p.txt | Louping ill virus, complete genome. |
| 800) | 18mers_human_m_virus_1MM_1170_p.txt | Modoc virus, genome. |
| 801) | 18mers_human_m_virus_1MM_1171_p.txt | Montana myotis leukoencephalitis virus, complete |
| | | genome. |
| 802) | 18mers_human_m_virus_1MM_1172_p.txt | Murray Valley encephalitis virus, complete genome. |
| 803) | 18mers_human_m_virus_1MM_1173_p.txt | Pestivirus Giraffe-1, complete genome. |
| 804) | 18mers_human_m_virus_1MM_1174_p.txt | Pestivirus Reindeer-1, complete genome. |
| 805) | 18mers_human_m_virus_1MM_1175_p.txt | Pestivirus type 2, complete genome. |
| 806) | 18mers_human_m_virus_1MM_1176_p.txt | Pestivirus type 3, complete genome. |
| 807) | 18mers_human_m_virus_1MM_1177_p.txt | Powassan virus, complete genome. |
| 808) | 18mers_human_m_virus_1MM_1178_p.txt | Rio Bravo virus, genome. |
| 809) | 18mers_human_m_virus_1MM_1179_p.txt | Tamana bat virus, genome. |
| 810) | 18mers_human_m_virus_1MM_1180_p.txt | Tick-borne encephalitis virus, complete genome. |
| 811) | 18mers_human_m_virus_1MM_1181_p.txt | West Nile virus, complete genome. |
| 812) | 18mers_human_m_virus_1MM_1182_p.txt | Yellow fever virus, complete genome. |
| 813) | 18mers_human_m_virus_1MM_1183_p.txt | Apple stem pitting virus, complete genome. |
| 814) | 18mers_human_m_virus_1MM_1184_p.txt | Banana mild mosaic virus, complete genome. |
| 815) | 18mers_human_m_virus_1MM_1185_p.txt | Cherry green ring mottle virus, complete genome. |
| 816) | 18mers_human_m_virus_1MM_1186_p.txt | Cherry necrotic rusty mottle virus, complete genome. |
| 817) | 18mers_human_m_virus_1MM_1187_p.txt | Rupestris stem pitting associated virus-1, complete |
| | | genome. |
| 818) | 18mers_human_m_virus_1MM_1188_p.txt | Chinese wheat mosaic virus RNA1, complete sequence. |
| 819) | 18mers_human_m_virus_1MM_1190_p.txt | Oat golden stripe virus RNA 1, complete sequence. |
| 820) | 18mers_human_m_virus_1MM_1192_p.txt | Soil-borne cereal mosaic virus RNA1, complete sequence. |
| 821) | 18mers_human_m_virus_1MM_1194_p.txt | Soil-borne wheat mosaic virus RNA1, complete sequence. |
| 822) | 18mers_human_m_virus_1MM_1196_p.txt | Sorghum chlorotic spot virus RNA 1, complete sequence. |
| 823) | 18mers_human_m_virus_1MM_1198_p.txt | Hepatitis E virus, complete genome. |
| 824) | 18mers_human_m_virus_1MM_1199_p.txt | Barley stripe mosaic virus RNA 1, complete sequence. |
| 825) | 18mers_human_m_virus_1MM_1202_p.txt | Raspberry bushy dwarf virus RNA 1, complete sequence. |
| 826) | 18mers_human_m_virus_1MM_1204_p.txt | Infectious flacherie virus, complete genome. |
| 827) | 18mers_human_m_virus_1MM_1205_p.txt | Perina nuda picorna-like virus, complete genome. |
| 828) | 18mers_human_m_virus_1MM_1206_p.txt | Sacbrood virus, complete genome. |
| 829) | 18mers_human_m_virus_1MM_1207_p.txt | Bacteriophage AP205 virion, complete genome. |
| 830) | 18mers_human_m_virus_1MM_1208_p.txt | Bacteriophage M11, complete genome. |
| 831) | 18mers_human_m_virus_1MM_1209_p.txt | Bacteriophage SP, complete genome. |
| 832) | 18mers_human_m_virus_1MM_1210_p.txt | Enterobacteria phage fr, complete genome. |
| 833) | 18mers_human_m_virus_1MM_1211_p.txt | Enterobacteria phage GA, complete genome. |
| 834) | 18mers_human_m_virus_1MM_1212_p.txt | Enterobacteria phage KU1, complete genome. |
| 835) | 18mers_human_m_virus_1MM_1213_p.txt | Enterobacteria phage MX1. |
| 836) | 18mers_human_m_virus_1MM_1214_p.txt | Enterobacteria phage NL95. |
| 837) | 18mers_human_m_virus_1MM_1215_p.txt | Enterobacterio phage MS2, complete genome. |
| 838) | 18mers_human_m_virus_1MM_1216_p.txt | Pseudomonas phage PP7, complete genome. |
| 839) | 18mers_human_m_virus_1MM_1217_p.txt | Barley yellow dwarf virus - GAV, complete genome. |
| 840) | 18mers_human_m_virus_1MM_1218_p.txt | Barley yellow dwarf virus - MAV, complete genome. |
| 841) | 18mers_human_m_virus_1MM_1219_p.txt | Barley yellow dwarf virus - PAV, complete genome. |
| 842) | 18mers_human_m_virus_1MM_1220_p.txt | Barley yellow dwarf virus - PAS, complete genome. |
| 843) | 18mers_human_m_virus_1MM_1221_p.txt | Bean leafroll virus, complete genome. |
| 844) | 18mers_human_m_virus_1MM_1222_p.txt | Beet chlorosis virus, complete genome. |
| 845) | 18mers_human_m_virus_1MM_1223_p.txt | Beet mild yellowing virus, complete genome. |
| 846) | 18mers_human_m_virus_1MM_1224_p.txt | Beet western yellows virus, complete genome. |
| 847) | 18mers_human_m_virus_1MM_1225_p.txt | Cereal yellow dwarf virus - RPS, complete genome. |
| 848) | 18mers_human_m_virus_1MM_1226_p.txt | Cereal yellow dwarf virus - RPV, complete genome. |
| 849) | 18mers_human_m_virus_1MM_1227_p.txt | Cucurbit aphid-borne yellows virus, complete genome. |
| 850) | 18mers_human_m_virus_1MM_1228_p.txt | Pea enation mosaic virus-1, complete genome. |
| 851) | 18mers_human_m_virus_1MM_1229_p.txt | Potato leafroll virus, complete genome. |
| 852) | 18mers_human_m_virus_1MM_1230_p.txt | Soybean dwarf virus, complete genome. |
| 853) | 18mers_human_m_virus_1MM_1231_p.txt | Sugarcane yellow leaf virus, complete genome. |
| 854) | 18mers_human_m_virus_1MM_1232_p.txt | Turnip yellows virus, complete genome. |
| 855) | 18mers_human_m_virus_1MM_1233_p.txt | Maize rayado fino virus, complete genome. |
| 856) | 18mers_human_m_virus_1MM_1234_p.txt | Oat blue dwarf virus, complete genome. |
| 857) | 18mers_human_m_virus_1MM_1235_p.txt | Poinsettia mosaic virus, complete genome. |
| 858) | 18mers_human_m_virus_1MM_1236_p.txt | Cryphonectria parasitica mitovirus 1-NB631, complete |
| | | genome. |
| 859) | 18mers_human_m_virus_1MM_1237_p.txt | Ophiostoma mitovirus 3a, complete genome. |
| 860) | 18mers_human_m_virus_1MM_1238_p.txt | Ophiostoma novo-ulmi mitovirus 4-Ld, complete genome. |
| 861) | 18mers_human_m_virus_1MM_1239_p.txt | Ophiostoma novo-ulmi mitovirus 5-Ld, complete genome. |
| 862) | 18mers_human_m_virus_1MM_1240_p.txt | Ophiostoma novo-ulmi mitovirus 6-Ld, complete genome. |
| 863) | 18mers_human_m_virus_1MM_1241_p.txt | Saccharomyces cerevisiae narnavirus 20S RNA |
| | | &lt; W&gt;, complete genome. |
| 864) | 18mers_human_m_virus_1MM_1242_p.txt | Saccharomyces cerevisiae narnavirus 23S RNA |
| | | &lt; T&gt;, complete genome. |
| 865) | 18mers_human_m_virus_1MM_1243_p.txt | Avian infectious bronchitis virus, complete genome. |
| 866) | 18mers_human_m_virus_1MM_1244_p.txt | Bovine coronavirus, complete genome. |
| 867) | 18mers_human_m_virus_1MM_1245_p.txt | Equine arteritis virus, complete genome. |
| 868) | 18mers_human_m_virus_1MM_1246_p.txt | Human coronavirus 229E, complete genome. |
| 869) | 18mers_human_m_virus_1MM_1247_p.txt | Lactate dehydrogenase-elevating virus, complete genome. |
| 870) | 18mers_human_m_virus_1MM_1248_p.txt | Murine hepatitis virus, complete genome. |
| 871) | 18mers_human_m_virus_1MM_1249_p.txt | Porcine epidemic diarrhea virus, complete genome. |
| 872) | 18mers_human_m_virus_1MM_1250_p.txt | Porcine reproductive and respiratory syndrome virus, |
| | | complete genome. |
| 873) | 18mers_human_m_virus_1MM_1251_p.txt | SARS coronavirus, complete genome. |
| 874) | 18mers_human_m_virus_1MM_1252_p.txt | Simian hemorrhagic fever virus, complete genome. |
| 875) | 18mers_human_m_virus_1MM_1253_p.txt | Transmissible gastroenteritis virus, complete genome. |
| 876) | 18mers_human_m_virus_1MM_1254_p.txt | Black beetle virus RNA 1, complete sequence. |
| 877) | 18mers_human_m_virus_1MM_1256_p.txt | Boolarra virus, complete genome. |
| 878) | 18mers_human_m_virus_1MM_1258_p.txt | Epinephelus tauvina nervous necrosis virus RNA 1, |
| | | complete sequence. |
| 879) | 18mers_human_m_virus_1MM_1260_p.txt | Flock house virus, complete genome. |
| 880) | 18mers_human_m_virus_1MM_1262_p.txt | Nodamura virus RNA 1, complete sequence. |
| 881) | 18mers_human_m_virus_1MM_1264_p.txt | Pariacoto virus RNA1, complete sequence. |
| 882) | 18mers_human_m_virus_1MM_1266_p.txt | Striped Jack nervous necrosis virus RNA1, complete |
| | | sequence. |
| 883) | 18mers_human_m_virus_1MM_1268_p.txt | Indian peanut clump virus RNA 1, complete sequence. |
| 884) | 18mers_human_m_virus_1MM_1270_p.txt | Peanut clump virus RNA 1, complete sequence. |
| 885) | 18mers_human_m_virus_1MM_1272_p.txt | Aichi virus, complete genome. |
| 886) | 18mers_human_m_virus_1MM_1273_p.txt | Avian encephalomyelitis virus, complete genome. |
| 887) | 18mers_human_m_virus_1MM_1274_p.txt | Bovine enterovirus, complete genome. |
| 888) | 18mers_human_m_virus_1MM_1275_p.txt | Bovine kobuvirus, complete genome. |
| 889) | 18mers_human_m_virus_1MM_1276_p.txt | Encephalomyocarditis virus, complete genome. |
| 890) | 18mers_human_m_virus_1MM_1277_p.txt | Enterovirus Yanbian 96-83csf, complete genome. |
| 891) | 18mers_human_m_virus_1MM_1278_p.txt | Equine rhinitis A virus, complete genome. |
| 892) | 18mers_human_m_virus_1MM_1279_p.txt | Equine rhinitis B virus, complete genome. |
| 893) | 18mers_human_m_virus_1MM_1280_p.txt | Equine rhinovirus 3, complete genome. |
| 894) | 18mers_human_m_virus_1MM_1281_p.txt | Foot-and-mouth disease virus C, complete genome. |
| 895) | 18mers_human_m_virus_1MM_1282_p.txt | Foot-and-mouth disease virus O, complete genome. |
| 896) | 18mers_human_m_virus_1MM_1283_p.txt | Foot-and-mouth disease virus SAT 2, complete genome. |
| 897) | 18mers_human_m_virus_1MM_1284_p.txt | Hepatitis A virus, complete genome. |
| 898) | 18mers_human_m_virus_1MM_1285_p.txt | Human echovirus I, complete genome. |
| 899) | 18mers_human_m_virus_1MM_1286_p.txt | Human enterovirus A, complete genome. |
| 900) | 18mers_human_m_virus_1MM_1287_p.txt | Human enterovirus B, complete genome. |
| 901) | 18mers_human_m_virus_1MM_1288_p.txt | Human enterovirus C, complete genome. |
| 902) | 18mers_human_m_virus_1MM_1289_p.txt | Human enterovirus D, complete genome. |
| 903) | 18mers_human_m_virus_1MM_1290_p.txt | Human enterovirus E, complete genome. |
| 904) | 18mers_human_m_virus_1MM_1291_p.txt | Human parechovirus 2, complete genome. |
| 905) | 18mers_human_m_virus_1MM_1292_p.txt | Human rhinovirus 89, complete genome. |
| 906) | 18mers_human_m_virus_1MM_1293_p.txt | Human rhinovirus B, complete genome. |
| 907) | 18mers_human_m_virus_1MM_1294_p.txt | Ljungan virus, complete genome. |
| 908) | 18mers_human_m_virus_1MM_1295_p.txt | Poliovirus, complete genome. |
| 909) | 18mers_human_m_virus_1MM_1296_p.txt | Porcine enterovirus A, complete genome. |
| 910) | 18mers_human_m_virus_1MM_1297_p.txt | Porcine enterovirus B, complete genome. |
| 911) | 18mers_human_m_virus_1MM_1298_p.txt | Porcine teschovirus I, complete genome. |
| 912) | 18mers_human_m_virus_1MM_1299_p.txt | Simian picornavirus 1, complete genome. |
| 913) | 18mers_human_m_virus_1MM_1300_p.txt | Theilovirus, complete genome. |
| 914) | 18mers_human_m_virus_1MM_1301_p.txt | Bamboo mosaic virus, complete genome. |
| 915) | 18mers_human_m_virus_1MM_1302_p.txt | Cassava common mosaic virus, complete genome. |
| 916) | 18mers_human_m_virus_1MM_1303_p.txt | Clover yellow mosaic virus, complete genome. |
| 917) | 18mers_human_m_virus_1MM_1304_p.txt | Cymbidium mosaic virus, complete genome. |
| 918) | 18mers_human_m_virus_1MM_1305_p.txt | Foxtail mosaic virus, complete genome. |
| 919) | 18mers_human_m_virus_1MM_1306_p.txt | Narcissus mosaic virus, complete genome. |
| 920) | 18mers_human_m_virus_1MM_1307_p.txt | Papaya mosaic virus, complete genome. |
| 921) | 18mers_human_m_virus_1MM_1308_p.txt | Pepino mosaic virus, complete genome. |
| 922) | 18mers_human_m_virus_1MM_1309_p.txt | Plantago asiatica mosaic virus, complete genome. |
| 923) | 18mers_human_m_virus_1MM_1310_p.txt | Potato aucuba mosaic virus, complete genome. |
| 924) | 18mers_human_m_virus_1MM_1311_p.txt | Potato virus X, complete genome. |
| 925) | 18mers_human_m_virus_1MM_1312_p.txt | Scallion virus X, complete genome. |
| 926) | 18mers_human_m_virus_1MM_1313_p.txt | Strawberry mild yellow edge virus, complete genome. |
| 927) | 18mers_human_m_virus_1MM_1314_p.txt | Tulip virus X, complete genome. |
| 928) | 18mers_human_m_virus_1MM_1315_p.txt | White clover mosaic virus, complete genome. |
| 929) | 18mers_human_m_virus_1MM_1316_p.txt | Barley mild mosaic virus RNA 1, complete sequence. |
| 930) | 18mers_human_m_virus_1MM_1318_p.txt | Barley yellow mosaic virus RNA 1, complete sequence. |
| 931) | 18mers_human_m_virus_1MM_1320_p.txt | Bean common mosaic necrosis virus, complete genome. |
| 932) | 18mers_human_m_virus_1MM_1321_p.txt | Bean common mosaic virus, complete genome. |
| 933) | 18mers_human_m_virus_1MM_1322_p.txt | Bean yellow mosaic virus, complete genome. |
| 934) | 18mers_human_m_virus_1MM_1323_p.txt | Brome streak mosaic virus, complete genome. |
| 935) | 18mers_human_m_virus_1MM_1324_p.txt | Clover yellow vein virus, complete genome. |
| 936) | 18mers_human_m_virus_1MM_1325_p.txt | Cocksfoot streak virus, complete genome. |
| 937) | 18mers_human_m_virus_1MM_1326_p.txt | Cowpea aphid-borne mosaic virus, complete genome. |
| 938) | 18mers_human_m_virus_1MM_1327_p.txt | Dasheen mosaic virus, complete genome. |
| 939) | 18mers_human_m_virus_1MM_1328_p.txt | Japanese yam mosaic virus, complete genome. |
| 940) | 18mers_human_m_virus_1MM_1329_p.txt | Johnsongrass mosaic virus, complete genome. |
| 941) | 18mers_human_m_virus_1MM_1330_p.txt | Leek yellow stripe potyvirus, complete genome. |
| 942) | 18mers_human_m_virus_1MM_1331_p.txt | Lettuce mosaic virus, complete genome. |
| 943) | 18mers_human_m_virus_1MM_1332_p.txt | Maize dwarf mosaic virus, complete genome. |
| 944) | 17mers_human_m_virus_1MM_1333_p.txt | Oat mosaic virus RNA 1, complete |
| | | sequence. |
| 945) | 17mers_human_m_virus_1MM_1335_p.txt | Papaya ringspot virus, complete genome. |
| 946) | 17mers_human_m_virus_1MM_1336_p.txt | Pea seed-borne mosaic virus, complete |
| | | genome. |
| 947) | 17mers_human_m_virus_1MM_1337_p.txt | Peanut mottle virus, complete genome. |
| 948) | 17mers_human_m_virus_1MM_1338_p.txt | Pepper mottle virus, complete genome. |
| 949) | 17mers_human_m_virus_1MM_1339_p.txt | Peru tomato mosaic virus, complete |
| | | genome. |
| 950) | 17mers_human_m_virus_1MM_1340_p.txt | Potato virus V, complete genome. |
| 951) | 17mers_human_m_virus_1MM_1341_p.txt | Potato virus Y, complete genome. |
| 952) | 17mers_human_m_virus_1MM_1342_p.txt | Ryegrass mosaic virus, complete genome. |
| 953) | 17mers_human_m_virus_1MM_1343_p.txt | Scallion mosaic virus, complete genome. |
| 954) | 17mers_human_m_virus_1MM_1344_p.txt | Sorghum mosaic virus, complete genome. |
| 955) | 17mers_human_m_virus_1MM_1345_p.txt | Soybean mosaic virus, complete genome. |
| 956) | 17mers_human_m_virus_1MM_1346_p.txt | Sugarcane mosaic virus, complete genome. |
| 957) | 17mers_human_m_virus_1MM_1347_p.txt | Sweet potato feathery mottle virus, |
| | | complete genome. |
| 958) | 17mers_human_m_virus_1MM_1348_p.txt | Sweet potato mild mottle virus, complete |
| | | genome. |
| 959) | 17mers_human_m_virus_1MM_1349_p.txt | Tobacco etch virus, complete genome. |
| 960) | 17mers_human_m_virus_1MM_1350_p.txt | Tobacco vein mottling virus, complete |
| | | genome. |
| 961) | 17mers_human_m_virus_1MM_1351_p.txt | Turnip mosaic virus, complete genome. |
| 962) | 17mers_human_m_virus_1MM_1352_p.txt | Wheat streak mosaic virus, complete |
| | | genome. |
| 963) | 17mers_human_m_virus_1MM_1353_p.txt | Wheat yellow mosaic virus RNA 1, |
| | | complete sequence. |
| 964) | 17mers_human_m_virus_1MM_1355_p.txt | Wild potato mosaic virus, complete |
| | | genome. |
| 965) | 17mers_human_m_virus_1MM_1356_p.txt | Yam mosaic virus, complete genome. |
| 966) | 17mers_human_m_virus_1MM_1357_p.txt | Zucchini yellow mosaic virus, complete |
| | | genome. |
| 967) | 17mers_human_m_virus_1MM_1358_p.txt | Apple latent spherical virus segment 1, |
| | | complete sequence. |
| 968) | 17mers_human_m_virus_1MM_1360_p.txt | Maize chlorotic dwarf virus, complete |
| | | genome. |
| 969) | 17mers_human_m_virus_1MM_1361_p.txt | Parsnip yellow fleck virus, complete |
| | | genome. |
| 970) | 17mers_human_m_virus_1MM_1362_p.txt | Rice tungro spherical virus, complete |
| | | genome. |
| 971) | 17mers_human_m_virus_1MM_1363_p.txt | Satsuma dwarf virus RNA 1, complete |
| | | sequence. |
| 972) | 17mers_human_m_virus_1MM_1365_p.txt | Strawberry mottle virus RNA2, complete |
| | | sequence. |
| 973) | 17mers_human_m_virus_1MM_1367_p.txt | Cocksfoot mottle virus, complete genome. |
| 974) | 17mers_human_m_virus_1MM_1368_p.txt | Lucerne transient streak virus, complete |
| | | genome. |
| 975) | 17mers_human_m_virus_1MM_1369_p.txt | Rice yellow mottle virus, complete |
| | | genome. |
| 976) | 17mers_human_m_virus_1MM_1370_p.txt | Sesbania mosaic virus, complete genome. |
| 977) | 17mers_human_m_virus_1MM_1371_p.txt | Southern bean mosaic virus, complete |
| | | genome. |
| 978) | 17mers_human_m_virus_1MM_1372_p.txt | Southern cowpea mosaic virus, complete |
| | | genome. |
| 979) | 17mers_human_m_virus_1MM_1373_p.txt | Subterranean clover mottle virus, complete |
| | | genome. |
| 980) | 17mers_human_m_virus_1MM_1374_p.txt | Turnip rosette virus, complete genome. |
| 981) | 17mers_human_m_virus_1MM_1375_p.txt | Helicoverpa armigera stunt virus RNA 1, |
| | | complete sequence. |
| 982) | 17mers_human_m_virus_1MM_1377_p.txt | Nudaurelia capensis beta virus, complete |
| | | genome. |
| 983) | 17mers_human_m_virus_1MM_1378_p.txt | Crucifer tobamovirus, complete genome. |
| 984) | 17mers_human_m_virus_1MM_1379_p.txt | Cucumber fruit mottle mosaic virus, |
| | | complete genome. |
| 985) | 17mers_human_m_virus_1MM_1380_p.txt | Cucumber green mottle mosaic virus, |
| | | complete genome. |
| 986) | 17mers_human_m_virus_1MM_1381_p.txt | Kyuri green mottle mosaic virus, complete |
| | | genome. |
| 987) | 17mers_human_m_virus_1MM_1382_p.txt | Obuda pepper virus, complete genome. |
| 988) | 17mers_human_m_virus_1MM_1383_p.txt | Odontoglossum ringspot virus, complete |
| | | genome. |
| 989) | 17mers_human_m_virus_1MM_1384_p.txt | Paprika mild mottle virus, complete |
| | | genome. |
| 990) | 17mers_human_m_virus_1MM_1385_p.txt | Pepper mild mottle virus, complete |
| | | genome. |
| 991) | 17mers_human_m_virus_1MM_1386_p.txt | Ribgrass mosaic virus, complete genome. |
| 992) | 17mers_human_m_virus_1MM_1387_p.txt | Tobacco mild green mosaic virus, complete |
| | | genome. |
| 993) | 17mers_human_m_virus_1MM_1388_p.txt | Tobacco mosaic virus, complete genome. |
| 994) | 17mers_human_m_virus_1MM_1389_p.txt | Tomato mosaic virus, complete genome. |
| 995) | 17mers_human_m_virus_1MM_1390_p.txt | Turnip vein-clearing virus, complete |
| | | genome. |
| 996) | 17mers_human_m_virus_1MM_1391_p.txt | Youcai mosaic virus, complete genome. |
| 997) | 17mers_human_m_virus_1MM_1392_p.txt | Zucchini green mottle mosaic virus, |
| | | complete genome. |
| 998) | 17mers_human_m_virus_1MM_1393_p.txt | Pea early browning virus RNA 1, complete |
| | | sequence. |
| 999) | 17mers_human_m_virus_1MM_1395_p.txt | Pepper ringspot virus RNA 1, complete |
| | | sequence. |
| 1000) | 17mers_human_m_virus_1MM_1397_p.txt | Tobacco rattle virus RNA 1, complete |
| | | sequence. |
| 1001) | 17mers_human_m_virus_1MM_1399_p.txt | Aura virus, complete genome. |
| 1002) | 17mers_human_m_virus_1MM_1400_p.txt | Barmah Forest virus, complete genome. |
| 1003) | 17mers_human_m_virus_1MM_1401_p.txt | Chikungunya virus, complete genome. |
| 1004) | 17mers_human_m_virus_1MM_1402_p.txt | Eastern equine encephalitis virus, complete |
| | | genome. |
| 1005) | 17mers_human_m_virus_1MM_1403_p.txt | Igbo Ora virus, complete genome. |
| 1006) | 17mers_human_m_virus_1MM_1404_p.txt | Mayaro virus, complete genome. |
| 1007) | 17mers_human_m_virus_1MM_1405_p.txt | O'nyong-nyong virus, complete genome. |
| 1008) | 17mers_human_m_virus_1MM_1406_p.txt | Ross River virus, complete genome. |
| 1009) | 17mers_human_m_virus_1MM_1407_p.txt | Rubella virus, complete genome. |
| 1010) | 17mers_human_m_virus_1MM_1408_p.txt | Salmon pancreas disease virus, complete |
| | | genome. |
| 1011) | 17mers_human_m_virus_1MM_1409_p.txt | Semliki forest virus, complete genome. |
| 1012) | 17mers_human_m_virus_1MM_1410_p.txt | Sindbis virus, complete genome. |
| 1013) | 17mers_human_m_virus_1MM_1411_p.txt | Sleeping disease virus, complete genome. |
| 1014) | 17mers_human_m_virus_1MM_1412_p.txt | Venezuelan equine encephalitis virus, |
| | | complete genome. |
| 1015) | 17mers_human_m_virus_1MM_1413_p.txt | Western equine encephalomyelitis virus, |
| | | complete genome. |
| 1016) | 17mers_human_m_virus_1MM_1414_p.txt | Artichoke mottled crinkle virus, complete |
| | | genome. |
| 1017) | 17mers_human_m_virus_1MM_1415_p.txt | Beet black scorch virus, complete genome. |
| 1018) | 17mers_human_m_virus_1MM_1416_p.txt | Cardamine chlorotic fleck virus, complete |
| | | genome. |
| 1019) | 17mers_human_m_virus_1MM_1417_p.txt | Carnation Italian ringspot virus, complete |
| | | genome. |
| 1020) | 17mers_human_m_virus_1MM_1418_p.txt | Carnation mottle virus, complete genome. |
| 1021) | 17mers_human_m_virus_1MM_1419_p.txt | Carnation ringspot virus RNA 1, complete |
| | | sequence. |
| 1022) | 17mers_human_m_virus_1MM_1421_p.txt | Cowpea mottle virus, complete genome. |
| 1023) | 17mers_human_m_virus_1MM_1422_p.txt | Cucumber Bulgarian virus, complete |
| | | genome. |
| 1024) | 17mers_human_m_virus_1MM_1423_p.txt | Cucumber necrosis virus, complete |
| | | genome. |
| 1025) | 17mers_human_m_virus_1MM_1424_p.txt | Cymbidium ringspot virus, complete |
| | | genome. |
| 1026) | 17mers_human_m_virus_1MM_1425_p.txt | Galinsoga mosaic virus, complete genome. |
| 1027) | 17mers_human_m_virus_1MM_1426_p.txt | Hibiscus chlorotic ringspot virus, complete |
| | | genome. |
| 1028) | 17mers_human_m_virus_1MM_1427_p.txt | Japanese iris necrotic ring virus, complete |
| | | genome. |
| 1029) | 17mers_human_m_virus_1MM_1428_p.txt | Leek white stripe virus, complete genome. |
| 1030) | 17mers_human_m_virus_1MM_1429_p.txt | Maize chlorotic mottle virus, complete |
| | | genome. |
| 1031) | 17mers_human_m_virus_1MM_1430_p.txt | Melon necrotic spot virus, genome. |
| 1032) | 17mers_human_m_virus_1MM_1431_p.txt | Oat chlorotic stunt virus, complete |
| | | genome. |
| 1033) | 17mers_human_m_virus_1MM_1432_p.txt | Olive latent virus 1, complete genome. |
| 1034) | 17mers_human_m_virus_1MM_1433_p.txt | Panicum mosaic virus, complete genome. |
| 1035) | 17mers_human_m_virus_1MM_1434_p.txt | Pear latent virus, complete genome. |
| 1036) | 17mers_human_m_virus_1MM_1435_p.txt | Pothos latent virus, complete genome. |
| 1037) | 17mers_human_m_virus_1MM_1436_p.txt | Red clover necrotic mosaic virus RNA 1, |
| | | complete sequence. |
| 1038) | 17mers_human_m_virus_1MM_1438_p.txt | Saguaro cactus virus, complete genome. |
| 1039) | 17mers_human_m_virus_1MM_1439_p.txt | Sweet clover necrotic mosaic virus RNA 1, |
| | | complete sequence. |
| 1040) | 17mers_human_m_virus_1MM_1441_p.txt | Tobacco necrosis virus A, complete |
| | | genome. |
| 1041) | 17mers_human_m_virus_1MM_1442_p.txt | Tobacco necrosis virus D, complete |
| | | genome. |
| 1042) | 17mers_human_m_virus_1MM_1443_p.txt | Tomato bushy stunt virus, complete |
| | | genome. |
| 1043) | 17mers_human_m_virus_1MM_1444_p.txt | Turnip crinkle virus, complete genome. |
| 1044) | 17mers_human_m_virus_1MM_1445_p.txt | Apple chlorotic leaf spot virus, complete |
| | | genome. |
| 1045) | 17mers_human_m_virus_1MM_1446_p.txt | Cherry mottle leaf virus, complete genome. |
| 1046) | 17mers_human_m_virus_1MM_1447_p.txt | Chayote mosaic tymovirus, complete |
| | | genome. |
| 1047) | 17mers_human_m_virus_1MM_1448_p.txt | Eggplant mosaic virus, complete genome. |
| 1048) | 17mers_human_m_virus_1MM_1449_p.txt | Erysimum latent virus, complete genome. |
| 1049) | 17mers_human_m_virus_1MM_1450_p.txt | Kennedya yellow mosaic virus, complete |
| | | genome. |
| 1050) | 17mers_human_m_virus_1MM_1451_p.txt | Ononis yellow mosaic virus, complete |
| | | genome. |
| 1051) | 17mers_human_m_virus_1MM_1452_p.txt | Physalis mottle virus, complete genome. |
| 1052) | 17mers_human_m_virus_1MM_1453_p.txt | Turnip yellow mosaic virus, complete |
| | | genome. |
| 1053) | 17mers_human_m_virus_1MM_1454_p.txt | Carrot mottle mimic virus, complete |
| | | genome. |
| 1054) | 17mers_human_m_virus_1MM_1455_p.txt | Groundnut rosette virus, complete genome. |
| 1055) | 17mers_human_m_virus_1MM_1456_p.txt | Pea enation mosaic virus-2, complete |
| | | genome. |
| 1056) | 17mers_human_m_virus_1MM_1457_p.txt | Tobacco bushy top virus, complete |
| | | genome. |
| 1057) | 17mers_human_m_virus_1MM_1458_p.txt | Grapevine virus A, complete genome. |
| 1058) | 17mers_human_m_virus_1MM_1459_p.txt | Grapevine virus B, complete genome. |
| 1059) | 17mers_human_m_virus_1MM_1460_p.txt | Acyrthosiphon pisum virus, complete |
| | | genome. |
| 1060) | 17mers_human_m_virus_1MM_1461_p.txt | Beet western yellows ST9 associated virus, |
| | | complete genome. |
| 1061) | 17mers_human_m_virus_1MM_1462_p.txt | Botrytis virus F, complete genome. |
| 1062) | 17mers_human_m_virus_1MM_1463_p.txt | Citrus leaf blotch virus, complete genome. |
| 1063) | 17mers_human_m_virus_1MM_1464_p.txt | Euprosterna elaeasa virus, complete |
| | | genome. |
| 1064) | 17mers_human_m_virus_1MM_1465_p.txt | Grapevine fleck virus, complete genome. |
| 1065) | 17mers_human_m_virus_1MM_1466_p.txt | Indian citrus ringspot virus, complete |
| | | genome. |
| 1066) | 17mers_human_m_virus_1MM_1467_p.txt | Oyster mushroom spherical virus, complete |
| | | genome. |
| 1067) | 17mers_human_m_virus_1MM_1468_p.txt | Sugarcane striate mosaic associated virus, |
| | | complete genome. |
| 1068) | 17mers_human_m_virus_1MM_1469_p.txt | Bacteriophage IN93, complete genome. |
| 1069) | 17mers_human_m_virus_1MM_1470_p.txt | Bacteriophage L5, complete genome. |
| 1070) | 17mers_human_m_virus_1MM_1471_p.txt | Bacteriophage Mx8, complete genome. |
| 1071) | 17mers_human_m_virus_1MM_1472_p.txt | Mycobacteriophage Barnyard virion, |
| | | complete genome. |
| 1072) | 18mers_human_m_virus_2MM_1473_p.txt | Mycobacteriophage Bxz1 virion, complete |
| | | genome. |
| 1073) | 17mers_human_m_virus_1MM_1474_p.txt | Mycobacteriophage Bxz2 virion, complete |
| | | genome. |
| 1074) | 17mers_human_m_virus_1MM_1475_p.txt | Mycobacteriophage Che8 virion, complete |
| | | genome. |
| 1075) | 17mers_human_m_virus_1MM_1476_p.txt | Mycobacteriophage Che9c virion, |
| | | complete genome. |
| 1076) | 17mers_human_m_virus_1MM_1477_p.txt | Mycobacteriophage Che9d virion, |
| | | complete genome. |
| 1077) | 17mers_human_m_virus_1MM_1478_p.txt | Mycobacteriophage CJW1 virion, complete |
| | | genome. |
| 1078) | 17mers_human_m_virus_1MM_1479_p.txt | Mycobacteriophage Corndog virion, |
| | | complete genome. |
| 1079) | 17mers_human_m_virus_1MM_1480_p.txt | Mycobacteriophage Omega virion, |
| | | complete genome. |
| 1080) | 17mers_human_m_virus_1MM_1481_p.txt | Mycobacteriophage Rosebush virion, |
| | | complete genome. |
| 1081) | 17mers_human_m_virus_1MM_1482_p.txt | Sinorhizobium meliloti phage PBC5, |
| | | complete genome. |
| 1082) | 17mers_human_m_virus_1MM_1483_p.txt | Staphylococcus aureus phage phi 11 |
| | | provirus, complete genome. |
| 1083) | 17mers_human_m_virus_1MM_1484_p.txt | Staphylococcus aureus phage phi 12 |
| | | provirus, complete genome. |
| 1084) | 17mers_human_m_virus_1MM_1485_p.txt | Staphylococcus aureus phage phi 13 |
| | | provirus, complete genome. |
| 1085) | 17mers_human_m_virus_1MM_1486_p.txt | Staphylococcus phage phiN315 provirus, |
| | | complete genome. |
| 1086) | 17mers_human_m_virus_1MM_1487_p.txt | Streptococcus pyogenes phage 315.1 |
| | | provirus, complete genome. |
| 1087) | 17mers_human_m_virus_1MM_1488_p.txt | Streptococcus pyogenes phage 315.2 |
| | | provirus, complete genome. |
| 1088) | 17mers_human_m_virus_1MM_1489_p.txt | Streptococcus pyogenes phage 315.3 |
| | | provirus, complete genome. |
| 1089) | 17mers_human_m_virus_1MM_1490_p.txt | Streptococcus pyogenes phage 315.4 |
| | | provirus, complete genome. |
| 1090) | 17mers_human_m_virus_1MM_1491_p.txt | Streptococcus pyogenes phage 315.5 |
| | | provirus, complete genome. |
| 1091) | 17mers_human_m_virus_1MM_1492_p.txt | Streptococcus pyogenes phage 315.6 |
| | | provirus, complete genome. |
| 1092) | 17mers_human_m_virus_1MM_1493_p.txt | Carrot red leaf luteovirus associated RNA, |
| | | complete genome. |
| 1093) | 17mers_human_m_virus_1MM_1494_p.txt | Planaria asexual strain-specific virus-like |
| | | element type 1 large DNA |
| 1094) | 17mers_human_m_virus_1MM_1496_p.txt | Saccharomyces cerevisiae killer virus M1, |
| | | complete genome. |
| 1095) | 17mers_human_m_virus_1MM_1497_p.txt | Sclerophthora macrospora virus B, |
| | | complete genome. |

5) SEQ ID NO 8: Selected Microbe 17 mers - It contains 7 files, each having the
subsequences of length 17 that are present in one microbial genome, but are absent in
human genome and other microbial genomes in the list. Sequences produced from these
sequences with two mismatches are also not present in the human genome or the
genomes of other microbes in the list.

| | File name | Genome name |
|---|---|---|
| 1. | 17_h_m_2mm_m_2_65.txt | Agrobacterium tumefaciens strain C58 circular |
| | | chromosome |
| 2. | 17_h_m_2mm_m_2_142.txt | Bacillus subtilis complete genome. |
| 3. | 17_h_m_2mm_m_2_146.txt | Corynebacterium glutamicum ATCC 13032 |
| 4. | 17_h_m_2mm_m_2_139.txt | Escherichia coli K-12 MG1655 complete genome. |
| 5. | 17_h_m_2mm_m_2_23.txt | Helicobacter pylori |
| 6. | 17_h_m_2mm_m_2_46.txt | Pseudomonas aeruginosa PA01 |
| 7. | 17_h_m_2mm_m_2_149.txt | Pseudomonas putida KT2440 |

6) SEQ ID NO 9: Selected Microbe 18 mers - It contains 7 files, each having the
subsequences of length 18 that are present in one microbial genome, but are absent in
human genome and other microbial genomes in the list. Sequences produced from these
sequences with two mismatches are also not present in the human genome or the
genomes of other microbes in the list.

| | File name | Genome name |
|---|---|---|
| 1. | 18_h_m_2mm_m_2_65.txt | Agrobacterium tumefaciens strain C58 circular |
| | | chromosome |
| 2. | 18_h_m_2mm_m_2_142.txt | Bacillus subtilis complete genome. |
| 3. | 18_h_m_2mm_m_2_90.txt | Campylobacter jejuni complete genome. |
| 4. | 18_h_m_2mm_m_2_146.txt | Corynebacterium glutamicum ATCC 13032 |
| 5. | 18_h_m_2mm_m_2_139.txt | Escherichia coli K-12 MG1655 complete genome. |
| 6. | 18_h_m_2mm_m_2_23.txt | Helicobacter pylori |
| 7. | 18_h_m_2mm_m_2_46.txt | Pseudomonas aeruginosa PA01 |

CD4 contains two folders - SEQ ID NO 10: “Microbial 17 mers 2MM” and SEQ
ID NO 11: “Microbial 17 mers 1MM”. The contents of the folder are listed below.
1) SEQ ID NO 10: Folder Microbial 17 mers 2MM - It contains 146 files,
containing sequences of length 17, which are present in the microbial genome,
but are not present in the human genome. All sequences produced from these
sequences with two mismatches are also not present in the human genome. The
list below contains the file name and the corresponding microbial name.

| | File Name | Species Name |
|---|---|---|
| a. | 17mers_human_m_2MM_1_p.txt | Aeropyrum pernix K1 complete genome. |
| b. | 17mers_human_m_2MM_10_p.txt | Aquifex aeolicus complete genome. |
| c. | 17mers_human_m_2MM_100_p.txt | Nostoc sp. PCC 7120 plasmid |
| | | pCC7120delta DNA |
| d. | 17mers_human_m_2MM_102_p.txt | Nostoc sp. PCC 7120 plasmid |
| | | pCC7120gamma DNA |
| e. | 17mers_human_m_2MM_103_p.txt | Nostoc sp. PCC 7120 plasmid pCC7120zeta |
| | | DNA |
| f. | 17mers_human_m_2MM_104_p.txt | Oceanobacillus iheyensis |
| g. | 17mers_human_m_2MM_105_p.txt | Pasteurella multocida PM70 complete |
| | | genome. |
| h. | 17mers_human_m_2MM_106_p.txt | Pseudomonas aeruginosa PA01 |
| i. | 17mers_human_m_2MM_107_p.txt | Pseudomonas putida KT2440 |
| j. | 17mers_human_m_2MM_108_p.txt | Pyrobaculum aerophilum strain IM2 |
| | | complete genome. |
| k. | 17mers_human_m_2MM_109_p.txt | Pyrococcus abyssi complete genome. |
| l. | 17mers_human_m_2MM_110_p.txt | Pyrococcus furiosus DSM 3638 |
| m. | 17mers_human_m_2MM_111_p.txt | Pyrococcus horikoshii OT3 complete |
| | | genome. |
| n. | 17mers_human_m_2MM_112_p.txt | Ralstonia solanacearum GM11000 |
| | | chromosome |
| o. | 17mers_human_m_2MM_113_p.txt | Ralstonia solanacearum GM11000 |
| | | megaplasmid |
| p. | 17mers_human_m_2MM_114_p.txt | Rickettsia conorii Malish 7 |
| q. | 17mers_human_m_2MM_115_p.txt | Rickettsia prowazekii strain Madrid E |
| r. | 17mers_human_m_2MM_116_p.txt | Salmonella enterica serovar Typhi |
| | | (Salmonella typhi) strain CT18 |
| s. | 17mers_human_m_2MM_117_p.txt | Salmonella enterica serovar Typhi |
| | | (Salmonella typhi) strain CT18 plasmid pHCM1 |
| t. | 17mers_human_m_2MM_118_p.txt | Salmonella enterica serovar Typhi |
| | | (Salmonella typhi) strain CT18 plasmid pHCM2 |
| u. | 17mers_human_m_2MM_119_p.txt | Salmonella typhimurium LT2 |
| v. | 17mers_human_m_2MM_12_p.txt | Archaeoglobus fulgidus complete genome. |
| w. | 17mers_human_m_2MM_120_p.txt | Salmonella typhimurium LT2 strain |
| | | SGSC1412 plasmid pSLT complete |
| x. | 17mers_human_m_2MM_121_p.txt | Shewanella oneidensis MR-1 complete |
| | | genome. |
| y. | 17mers_human_m_2MM_122_p.txt | Shewanella oneidensis MR-1 megaplasmid |
| | | complete sequence. |
| z. | 17mers_human_m_2MM_123_p.txt | Shigella flexneri 2a str. 301 complete |
| | | genome. |
| aa. | 17mers_human_m_2MM_124_p.txt | Sinorhizobium meliloti 1021 complete |
| | | genomes. |
| bb. | 17mers_human_m_2MM_125_p.txt | Sinorhizobium meliloti plasmid pSymA |
| | | complete plasmid sequence. |
| cc. | 17mers_human_m_2MM_126_p.txt | Sinorhizobium meliloti plasmid pSymB |
| dd. | 17mers_human_m_2MM_127_p.txt | Staphylococcus aureus MW2 |
| ee. | 17mers_human_m_2MM_128_p.txt | Staphylococcus aureus strain Mu50 |
| ff. | 17mers_human_m_2MM_13_p.txt | Bacillus halodurans C-125 |
| gg. | 17mers_human_m_2MM_130_p.txt | Staphylococcus aureus strain N315 |
| hh. | 17mers_human_m_2MM_131_p.txt | Staphylococcus aureus subsp. aureus N315 |
| | | plasmid pN315B DNA |
| ii. | 17mers_human_m_2MM_132_p.txt | Streptococcus agalactiae |
| jj. | 17mers_human_m_2MM_133_p.txt | Streptococcus agalactiae NEM316 |
| kk. | 17mers_human_m_2MM_134_p.txt | Streptococcus mutans UA159 complete |
| | | genome. |
| ll. | 17mers_human_m_2MM_135_p.txt | Streptococcus pneumoniae complete |
| | | genome. |
| mm. | 17mers_human_m_2MM_136_p.txt | Streptococcus pneumoniae R6 |
| | | complete genome. |
| nn. | 17mers_human_m_2MM_137_p.txt | Streptococcus pyogenes MGAS315 |
| oo. | 17mers_human_m_2MM_138_p.txt | Streptococcus pyogenes strain MGAS8232 |
| pp. | 17mers_human_m_2MM_139_p.txt | Streptococcus pyogenes strain SF370 |
| | | serotype M1 |
| qq. | 17mers_human_m_2MM_14_p.txt | Bacillus subtilis complete genome. |
| rr. | 17mers_human_m_2MM_140_p.txt | Streptomyces coelicolor A3(2) |
| ss. | 17mers_human_m_2MM_141_p.txt | Streptomyces coelicolor plasmid SCP1. |
| tt. | 17mers_human_m_2MM_142_p.txt | Streptomyces coelicolor plasmid SCP2. |
| uu. | 17mers_human_m_2MM_143_p.txt | Sulfolobus solfataricus complete genome. |
| vv. | 17mers_human_m_2MM_144_p.txt | Sulfolobus tokodaii complete genome. |
| ww. | 17mers_human_m_2MM_145_p.txt | Synechocystis PCC6803 complete genome. |
| xx. | 17mers_human_m_2MM_146_p.txt | Thermoanaerobacter tengcongensis strain |
| | | MB4T |
| yy. | 17mers_human_m_2MM_147_p.txt | Thermoplasma acidophilum |
| zz. | 17mers_human_m_2MM_148_p.txt | Thermoplasma volcanium |
| aaa. | 17mers_human_m_2MM_149_p.txt | Thermosynechococcus elongatus BP-1 |
| bbb. | 17mers_human_m_2MM_15_p.txt | Bifidobacterium longum NCC2705 |
| | | complete genome. |
| ccc. | 17mers_human_m_2MM_150_p.txt | Thermotoga maritima complete genome. |
| ddd. | 17mers_human_m_2MM_151_p.txt | Treponema pallidum complete genome. |
| eee. | 17mers_human_m_2MM_152_p.txt | Ureaplasma urealyticum complete genome. |
| fff. | 17mers_human_m_2MM_153_p.txt | Vibrio cholerae chromosome I |
| ggg. | 17mers_human_m_2MM_154_p.txt | Vibrio vulnificus CMCP6 chromosome I |
| | | complete sequence. |
| hhh. | 17mers_human_m_2MM_155_p.txt | Wigglesworthia brevipalpis |
| iii. | 17mers_human_m_2MM_157_p.txt | Xanthomonas campestris pv. campestris str. |
| | | ATCC 33913 |
| jjj. | 17mers_human_m_2MM_158_p.txt | Xylella fastidiosa |
| kkk. | 17mers_human_m_2MM_159_p.txt | Xylella fastidiosa plasmid pXF1.3 |
| lll. | 17mers_human_m_2MM_16_p.txt | Borrelia burgdorferi. |
| mmm. | 17mers_human_m_2MM_160_p.txt | Xylella fastidiosa plasmid pX F51 |
| nnn. | 17mers_human_m_2MM_162_p.txt | Yersinia pestis KIM complete genome. |
| ooo. | 17mers_human_m_2MM_163_p.txt | Yersinia pestis strain CO92 |
| ppp. | 17mers_human_m_2MM_164_p.txt | Yersinia pestis plasmid pCD1. |
| qqq. | 17mers_human_m_2MM_165_p.txt | Yersinia pestis plasmid pPCP1. |
| rrr. | 17mers_human_m_2MM_166_p.txt | Yersinia pestis plasmid pPMT1. |
| sss. | 17mers_human_m_2MM_168_p.txt | Nitrosomonas europaea |
| ttt. | 17mers_human_m_2MM_169_p.txt | Prochlorococcus marinus MED4 |
| uuu. | 17mers_human_m_2MM_170_p.txt | Prochlorococcus marinus MIT9313 |
| vvv. | 17mers_human_m_2MM_171_p.txt | Rhodopseudomonas palustris |
| www. | 17mers_human_m_2MM_172_p.txt | Staphylococcus epidermidis |
| | | ATCC 12228, complete genome |
| xxx. | 17mers_human_m_2MM_173_p.txt | Synechococcus |
| yyy. | 17mers_human_m_2MM_174_p.txt | Clostridium tetani E88, complete genome. |
| zzz. | 17mers_human_m_2MM_175_p.txt | Lactobacillus plantarum WCFS1, complete |
| | | genome. |
| aaaa. | 17mers_human_m_2MM_176_p.txt | Pseudomonas syringae pv. tomato |
| | | str. DC3000 |
| bbbb. | 17mers_human_m_2MM_177_p.txt | Streptococcus pyogenes phage |
| | | 315.3 provirus |
| cccc. | 17mers_human_m_2MM_178_p.txt | Tropheryma whipplei str. Twist |
| dddd. | 17mers_human_m_2MM_180_p.txt | Xylella fastidiosa Temecula1 |
| eeee. | 17mers_human_m_2MM_2_p.txt | Agrobacterium tumefaciens str. C58 linear |
| | | chromosome |
| ffff. | 17mers_human_m_2MM_3_p.txt | Agrobacterium tumefaciens str. C58 AT |
| | | plasmid complete sequence. |
| gggg. | 17mers_human_m_2MM_31_p.txt | Borrelia burgdorferi plasmid lp28-2 |
| hhhh. | 17mers_human_m_2MM_38_p.txt | Bradyrhizobium japonicum strain |
| | | USDA110 |
| iiii. | 17mers_human_m_2MM_39_p.txt | Brucella melitensis strain 16M chromosome I |
| jjjj. | 17mers_human_m_2MM_4_p.txt | Agrobacterium tumefaciens str. C58 Ti |
| | | plasmid |
| kkkk. | 17mers_human_m_2MM_40_p.txt | Brucella suis 1330 chromosome I complete |
| | | genome. |
| llll. | 17mers_human_m_2MM_41_p.txt | Buchnera aphidicola str. Sg (Schizaphis |
| | | graminum) complete genome. |
| mmmm. | 17mers_human_m_2MM_42_p.txt | Buchnera sp. APS complete genome. |
| nnnn. | 17mers_human_m_2MM_45_p.txt | Campylobacter jejuni complete genome. |
| oooo. | 17mers_human_m_2MM_46_p.txt | Caulobacter crescentus complete genome. |
| pppp. | 17mers_human_m_2MM_47_p.txt | Chlamydia muridarum |
| qqqq. | 17mers_human_m_2MM_49_p.txt | Chlamydia trachomatis complete genome. |
| rrrr. | 17mers_human_m_2MM_5_p.txt | Agrobacterium tumefaciens strain C58 |
| | | circular chromosome |
| ssss. | 17mers_human_m_2MM_50_p.txt | Chlamydophila pneumoniae AR39 |
| tttt. | 17mers_human_m_2MM_51_p.txt | Chlamydophila pneumoniae J138 |
| uuuu. | 17mers_human_m_2MM_52_p.txt | Chlorobium tepidum TLS complete |
| | | genome. |
| vvvv. | 17mers_human_m_2MM_53_p.txt | Clostridium acetobutylicum ATCC824 |
| | | complete genome. |
| wwww. | 17mers_human_m_2MM_55_p.txt | Clostridium perfringens 13 DNA |
| xxxx. | 17mers_human_m_2MM_57_p.txt | Corynebacterium efficiens YS-314 |
| yyyy. | 17mers_human_m_2MM_58_p.txt | Corynebacterium glutamicum ATCC 13032 |
| zzzz. | 17mers_human_m_2MM_59_p.txt | Deinococcus radiodurans R1 complete |
| | | chromosome I. |
| aaaaa. | 17mers_human_m_2MM_6_p.txt | Agrobacterium tumefaciens strain C58 |
| | | circular chromosome |
| bbbbb. | 17mers_human_m_2MM_60_p.txt | Deinococcus radiodurans R1 megaplasmid |
| | | MP1 |
| ccccc. | 17mers_human_m_2MM_61_p.txt | Deinococcus radiodurans R1 plasmid CP1 |
| ddddd. | 17mers_human_m_2MM_62_p.txt | Escherichia coli CFT073 complete genome. |
| eeeee. | 17mers_human_m_2MM_63_p.txt | Escherichia coli K-12 MG 1655 complete |
| | | genome. |
| fffff. | 17mers_human_m_2MM_64_p.txt | Escherichia coli O157:H7 |
| ggggg. | 17mers_human_m_2MM_65_p.txt | Escherichia coli O157:H7 |
| hhhhh. | 17mers_human_m_2MM_66_p.txt | Fusobacterium nucleatum subsp. nucleatum |
| | | ATCC 25586 |
| iiiii. | 17mers_human_m_2MM_67_p.txt | Haemophilus influenzae Rd complete |
| | | genome. |
| jjjjj. | 17mers_human_m_2MM_68_p.txt | Halobacterium sp. NRC-1 complete |
| | | genome. |
| kkkkk. | 17mers_human_m_2MM_69_p.txt | Halobacterium sp. NRC-1 plasmid |
| | | pNRC100 |
| lllll. | 17mers_human_m_2MM_7_p.txt | Agrobacterium tumefaciens strain C58 |
| | | linear chromosome |
| mmmmm. | 17mers_human_m_2MM_70_p.txt | Halobacterium sp. NRC-1 plasmid |
| | | pNRC200 complete genome. |
| nnnnn. | 17mers_human_m_2MM_71_p.txt | Helicobacter pylori |
| ooooo. | 17mers_human_m_2MM_72_p.txt | Helicobacter pylori 26695 complete |
| | | genome. |
| ppppp. | 17mers_human_m_2MM_73_p.txt | Lactococcus lactis subsp. lactis IL1403 |
| | | complete genome. |
| qqqqq. | 17mers_human_m_2MM_74_p.txt | Leptospira interrogans serovar lai str. 56601 |
| | | chromosome I |
| rrrrr. | 17mers_human_m_2MM_75_p.txt | Listeria innocua Clip11262 |
| sssss. | 17mers_human_m_2MM_76_p.txt | Listeria innocua plasmid pL1100 |
| ttttt. | 17mers_human_m_2MM_77_p.txt | Listeria monocytogenes strain EGD |
| uuuuu. | 17mers_human_m_2MM_78_p.txt | Mesorhizobium loti complete genome |
| vvvvv. | 17mers_human_m_2MM_79_p.txt | Mesorhizobium loti plasmid pMLa DNA |
| wwwww. | 17mers_human_m_2MM_8_p.txt | Agrobacterium tumefaciens strain C58 |
| | | plasmid AT |
| xxxxx. | 17mers_human_m_2MM_80_p.txt | Mesorhizobium loti plasmid pMLb DNA |
| yyyyy. | 17mers_human_m_2MM_81_p.txt | Methanobacterium thermoautotrophicum |
| | | delta H complete genome. |
| zzzzz. | 17mers_human_m_2MM_82_p.txt | Methanococcus jannaschii complete |
| | | genome. |
| aaaaaa. | 17mers_human_m_2MM_85_p.txt | Methanopyrus kandleri AV19 |
| bbbbbb. | 17mers_human_m_2MM_86_p.txt | Methanosarcina acetivorans str. C2A |
| cccccc. | 17mers_human_m_2MM_87_p.txt | Methanosarcina mazei strain Goe1 |
| dddddd. | 17mers_human_m_2MM_88_p.txt | Mycobacterium leprae strain TN complete |
| | | genome. |
| eeeeee. | 17mers_human_m_2MM_89_p.txt | Mycobacterium tuberculosis CDC1551 |
| ffffff. | 17mers_human_m_2MM_9_p.txt | Agrobacterium tumefaciens strain C58 |
| | | plasmid Ti |
| gggggg. | 17mers_human_m_2MM_90_p.txt | Mycobacterium tuberculosis complete |
| | | genome. |
| hhhhhh. | 17mers_human_m_2MM_91_p.txt | Mycoplasma genitalium G37 complete |
| | | genome. |
| iiiiii. | 17mers_human_m_2MM_93_p.txt | Mycoplasma pneumoniae M129 |
| jjjjjj. | 17mers_human_m_2MM_95_p.txt | Neisseria meningitidis serogroup A strain |
| | | Z2491 complete genome. |
| kkkkkk. | 17mers_human_m_2MM_96_p.txt | Neisseria meningitidis serogroup B strain |
| | | MC58 complete genome. |
| llllll. | 17mers_human_m_2MM_97_p.txt | Nostoc sp. PCC 7120 complete genome. |
| mmmmmm. | 17mers_human_m_2MM_98_p.txt | Nostoc sp. PCC 7120 plasmid |
| | | pCC7120alpha DNA |
| nnnnnn. | 17mers_human_m_2MM_99_p.txt | Nostoc sp. PCC 7120 plasmid |
| | | pCC7120beta DNA |

2) SEQ ID NO 11: Folder 17 mers 1MM - It contains 80 files, containing
sequences of length 17, which are present in the microbial genome, but are not
present in the human genome. All sequences produced from these sequences with
one mismatch are also not present in the human genome. There are 180 files in total
and this CD contains the first 80 of them. The list below contains the file name and
the corresponding microbial name.

| | File Name | Species Name |
|---|---|---|
| 1. | 17mers_human_m_1MM_1_p.txt | Aeropyrum pernix K1 complete genome. |
| 2. | 17mers_human_m_1MM_2_p.txt | Agrobacterium tumefaciens str. C58 linear |
| | | chromosome |
| 3. | 17mers_human_m_1MM_3_p.txt | Agrobacterium tumefaciens str. C58 AT |
| | | plasmid complete sequence. |
| 4. | 17mers_human_m_1MM_4_p.txt | Agrobacterium tumefaciens str. C58 Ti plasmid |
| 5. | 17mers_human_m_1MM_5_p.txt | Agrobacterium tumefaciens strain C58 circular |
| | | chromosome |
| 6. | 17mers_human_m_1MM_6_p.txt | Agrobacterium tumefaciens strain C58 circular |
| | | chromosome |
| 7. | 17mers_human_m_1MM_7_p.txt | Agrobacterium tumefaciens strain C58 linear |
| | | chromosome |
| 8. | 17mers_human_m_1MM_8_p.txt | Agrobacterium tumefaciens strain C58 plasmid |
| | | AT |
| 9. | 17mers_human_m_1MM_9_p.txt | Agrobacterium tumefaciens strain C58 plasmid |
| | | Ti |
| 10. | 17mers_human_m_1MM_10_p.txt | Aquifex aeolicus complete genome. |
| 11. | 17mers_human_m_1MM_11_p.txt | Aquifex aeolicus plasmid ece1 |
| 12. | 17mers_human_m_1MM_12_p.txt | Archaeoglobus fulgidus complete genome. |
| 13. | 17mers_human_m_1MM_13_p.txt | Bacillus halodurans C-125 |
| 14. | 17mers_human_m_1MM_14_p.txt | Bacillus subtilis complete genome. |
| 15. | 17mers_human_m_1MM_15_p.txt | Bifidobacterium longum NCC2705 complete |
| | | genome. |
| 16. | 17mers_human_m_1MM_16_p.txt | Borrelia burgdorferi. |
| 17. | 17mers_human_m_1MM_17_p.txt | Borrelia burgdorferi plasmid cp9 |
| 18. | 17mers_human_m_1MM_18_p.txt | Borrelia burgdorferi plasmid cp26 |
| 19. | 17mers_human_m_1MM_19_p.txt | Borrelia burgdorferi plasmid cp32-1 |
| 20. | 17mers_human_m_1MM_20_p.txt | Borrelia burgdorferi plasmid cp32-3 |
| 21. | 17mers_human_m_1MM_21_p.txt | Borrelia burgdorferi plasmid cp32-4 |
| 22. | 17mers_human_m_1MM_22_p.txt | Borrelia burgdorferi plasmid cp32-6 |
| 23. | 17mers_human_m_1MM_23_p.txt | Borrelia burgdorferi plasmid cp32-7 |
| 24. | 17mers_human_m_1MM_24_p.txt | Borrelia burgdorferi plasmid cp32-8 |
| 25. | 17mers_human_m_1MM_25_p.txt | Borrelia burgdorferi plasmid cp32-9 |
| 26. | 17mers_human_m_1MM_26_p.txt | Borrelia burgdorferi plasmid lp5 |
| 27. | 17mers_human_m_1MM_27_p.txt | Borrelia burgdorferi plasmid lp17 |
| 28. | 17mers_human_m_1MM_28_p.txt | Borrelia burgdorferi plasmid lp21 |
| 29. | 17mers_human_m_1MM_29_p.txt | Borrelia burgdorferi plasmid lp25 |
| 30. | 17mers_human_m_1MM_30_p.txt | Borrelia burgdorferi plasmid lp28-1 |
| 31. | 17mers_human_m_1MM_31_p.txt | Borrelia burgdorferi plasmid lp28-2 |
| 32. | 17mers_human_m_1MM_32_p.txt | Borrelia burgdorferi plasmid lp28-3 |
| 33. | 17mers_human_m_1MM_33_p.txt | Borrelia burgdorferi plasmid lp28-4 |
| 34. | 17mers_human_m_1MM_34_p.txt | Borrelia burgdorferi plasmid lp36 |
| 35. | 17mers_human_m_1MM_35_p.txt | Borrelia burgdorferi plasmid lp38 |
| 36. | 17mers_human_m_1MM_36_p.txt | Borrelia burgdorferi plasmid lp54 |
| 37. | 17mers_human_m_1MM_37_p.txt | Borrelia burgdorferi plasmid lp56 |
| 38. | 17mers_human_m_1MM_38_p.txt | Bradyrhizobium japonicum strain USDA110 |
| 39. | 17mers_human_m_1MM_39_p.txt | Brucella melitensis strain 16M chromosome I |
| 40. | 17mers_human_m_1MM_40_p.txt | Brucella suis 1330 chromosome I complete |
| | | genome. |
| 41. | 17mers_human_m_1MM_41_p.txt | Buchnera aphidicola str. Sg (Schizaphis |
| | | graminum) complete genome. |
| 42. | 17mers_human_m_1MM_42_p.txt | Buchnera sp. APS complete genome. |
| 43. | 17mers_human_m_1MM_43_p.txt | Buchnera sp. APS plasmid pLeu DNA |
| 44. | 17mers_human_m_1MM_44_p.txt | Buchnera sp. APS plasmid pTrp DNA |
| 45. | 17mers_human_m_1MM_45_p.txt | Campylobacter jejuni complete genome. |
| 46. | 17mers_human_m_1MM_46_p.txt | Caulobacter crescentus complete genome. |
| 47. | 17mers_human_m_1MM_47_p.txt | Chlamydia muridarum |
| 48. | 17mers_human_m_1MM_48_p.txt | Chlamydia muridarum plasmid pMoPn |
| 49. | 17mers_human_m_1MM_49_p.txt | Chlamydia trachomatis complete genome. |
| 50. | 17mers_human_m_1MM_50_p.txt | Chlamydophila pneumoniae AR39 |
| 51. | 17mers_human_m_1MM_51_p.txt | Chlamydophila pneumoniae J138 |
| 52. | 17mers_human_m_1MM_52_p.txt | Chlorobium tepidum TLS complete genome. |
| 53. | 17mers_human_m_1MM_53_p.txt | Clostridium acetobutylicum ATCC824 |
| | | complete genome. |
| 54. | 17mers_human_m_1MM_54_p.txt | Clostridium acetobutylicum megaplasmid |
| | | pSOL1 |
| 55. | 17mers_human_m_1MM_55_p.txt | Clostridium perfringens 13 DNA |
| 56. | 17mers_human_m_1MM_56_p.txt | Clostridium perfringens plasmid pCP13 DNA |
| 57. | 17mers_human_m_1MM_57_p.txt | Corynebacterium efficiens YS-314 |
| 58. | 17mers_human_m_1MM_58_p.txt | Corynebacterium glutamicum ATCC 13032 |
| 59. | 17mers_human_m_1MM_59_p.txt | Deinococcus radiodurans R1 complete |
| | | chromosome 1. |
| 60. | 17mers_human_m_1MM_60_p.txt | Deinococcus radiodurans R1 megaplasmid MP1 |
| 61. | 17mers_human_m_1MM_61_p.txt | Deinococcus radiodurans R1 plasmid CP1 |
| 62. | 17mers_human_m_1MM_62_p.txt | Escherichia coli CFT073 complete genome. |
| 63. | 17mers_human_m_1MM_63_p.txt | Escherichia coli K-12 MG1655 complete |
| | | genome. |
| 64. | 17mers_human_m_1MM_64_p.txt | Escherichia coli O157:H7 |
| 65. | 17mers_human_m_1MM_65_p.txt | Escherichia coli O157:H7 |
| 66. | 17mers_human_m_1MM_66_p.txt | Fusobacterium nucleatum subsp. nucleatum |
| | | ATCC 25586 |
| 67. | 17mers_human_m_1MM_67_p.txt | Haemophilus influenzae Rd complete genome. |
| 68. | 17mers_human_m_1MM_68_p.txt | Halobacterium sp. NRC-1 complete genome. |
| 69. | 17mers_human_m_1MM_69_p.txt | Halobacterium sp. NRC-1 plasmid pNRC100 |
| 70. | 17mers_human_m_1MM_70_p.txt | Halobacterium sp. NRC-1 plasmid pNRC200 |
| | | complete genome. |
| 71. | 17mers_human_m_1MM_71_p.txt | Helicobacter pylori |
| 72. | 17mers_human_m_1MM_72_p.txt | Helicobacter pylori 26695 complete genome. |
| 73. | 17mers_human_m_1MM_73_p.txt | Lactococcus lactis subsp. lactis IL1403 |
| | | complete genome. |
| 74. | 17mers_human_m_1MM_74_p.txt | Leptospira interrogans serovar lai str. 56601 |
| | | chromosome I |
| 75. | 17mers_human_m_1MM_75_p.txt | Listeria innocua Clip 11262 |
| 76. | 17mers_human_m_1MM_76_p.txt | Listeria innocua plasmid pLI100 |
| 77. | 17mers_human_m_1MM_77_p.txt | Listeria monocytogenes strain EGD |
| 78. | 17mers_human_m_1MM_78_p.txt | Mesorhizobium loti complete genome |
| 79. | 17mers_human_m_1MM_79_p.txt | Mesorhizobium loti plasmid pMLa DNA |
| 80. | 17mers_human_m_1MM_80_p.txt | Mesorhizobium loti plasmid pMLb DNA |
| 81. | | |

CD5 contains one folder - SEQ ID NO 12: “Microbial 17 mers 1MM”. The
contents of the folder are listed below.
SEQ ID NO 12: Folder 17 mers 1MM - It contains 60 files, containing sequences
of length 17, which are present in the microbial genome, but are not present in the
human genome. All sequences produced from these sequences with one mismatch
are also not present in the human genome. There are 180 files in total and this CD
contains the 81^{st} to 140^{th} of them. The list below contains the file name and the
corresponding microbial name.

| | File Name | Species Name |
|---|---|---|
| 82. | 17mers_human_m_1MM_81_p.txt | Methanobacterium thermoautotrophicum delta |
| | | H complete genome. |
| 83. | 17mers_human_m_1MM_82_p.txt | Methanococcus jannaschii complete genome. |
| 84. | 17mers_human_m_1MM_83_p.txt | Methanococcus jannaschii large extra- |
| | | chromosomal element |
| 85. | 17mers_human_m_1MM_84_p.txt | Methanococcus jannaschii small extra- |
| | | chromosomal element |
| 86. | 17mers_human_m_1MM_85_p.txt | Methanopyrus kandleri AV19 |
| 87. | 17mers_human_m_1MM_86_p.txt | Methanosarcina acetivorans str. C2A |
| 88. | 17mers_human_m_1MM_87_p.txt | Methanosarcina mazei strain Goe1 |
| 89. | 17mers_human_m_1MM_88_p.txt | Mycobacterium leprae strain TN complete |
| | | genome. |
| 90. | 17mers_human_m_1MM_89_p.txt | Mycobacterium tuberculosis CDC1551 |
| 91. | 17mers_human_m_1MM_90_p.txt | Mycobacterium tuberculosis complete genome. |
| 92. | 17mers_human_m_1MM_91_p.txt | Mycoplasma genitalium G37 complete genome. |
| 93. | 17mers_human_m_1MM_92_p.txt | Mycoplasma penetrans |
| 94. | 17mers_human_m_1MM_93_p.txt | Mycoplasma pneumoniae M129 |
| 95. | 17mers_human_m_1MM_94_p.txt | Mycoplasma pulmonis (strain UAB CTIP) |
| | | complete genome. |
| 96. | 17mers_human_m_1MM_95_p.txt | Neisseria meningitidis serogroup A strain |
| | | Z2491 complete genome. |
| 97. | 17mers_human_m_1MM_96_p.txt | Neisseria meningitidis serogroup B strain |
| | | MC58 complete genome. |
| 98. | 17mers_human_m_1MM_97_p.txt | Nostoc sp. PCC 7120 complete genome. |
| 99. | 17mers_human_m_1MM_98_p.txt | Nostoc sp. PCC 7120 plasmid pCC7120alpha |
| | | DNA |
| 100. | 17mers_human_m_1MM_99_p.txt | Nostoc sp. PCC 7120 plasmid |
| | | pCC7120beta DNA |
| 101. | 17mers_human_m_1MM_100_p.txt | Nostoc sp. PCC 7120 plasmid |
| | | pCC7120delta DNA |
| 102. | 17mers_human_m_1MM_101_p.txt | Nostoc sp. PCC 7120 plasmid |
| | | pCC7120epsilon DNA |
| 103. | 17mers_human_m_1MM_102_p.txt | Nostoc sp. PCC 7120 plasmid |
| | | pCC7120gamma DNA |
| 104. | 17mers_human_m_1MM_103_p.txt | Nostoc sp. PCC 7120 plasmid |
| | | pCC7120zeta DNA |
| 105. | 17mers_human_m_1MM_104_p.txt | Oceanobacillus iheyensis |
| 106. | 17mers_human_m_1MM_105_p.txt | Pasteurella multocida PM70 complete |
| | | genome. |
| 107. | 17mers_human_m_1MM_106_p.txt | Pseudomonas aeruginosa PA01 |
| 108. | 17mers_human_m_1MM_107_p.txt | Pseudomonas putida KT2440 |
| 109. | 17mers_human_m_1MM_108_p.txt | Pyrobaculum aerophilum strain 1M2 |
| | | complete genome. |
| 110. | 17mers_human_m_1MM_109_p.txt | Pyrococcus abyssi complete genome. |
| 111. | 17mers_human_m_1MM_110_p.txt | Pyrococcus furiosus DSM 3638 |
| 112. | 17mers_human_m_1MM_111_p.txt | Pyrococcus horikoshii OT3 complete |
| | | genome. |
| 113. | 17mers_human_m_1MM_112_p.txt | Ralstonia solanacearum GMI1000 |
| | | chromosome |
| 114. | 17mers_human_m_1MM_113_p.txt | Ralstonia solanacearum GMI1000 |
| | | megaplasmid |
| 115. | 17mers_human_m_1MM_114_p.txt | Rickettsia conorii Malish 7 |
| 116. | 17mers_human_m_1MM_115_p.txt | Rickettsia prowazekii strain Madrid E |
| 117. | 17mers_human_m_1MM_116_p.txt | Salmonella enterica serovar Typhi |
| | | (Salmonella typhi) strain CT18 |
| 118. | 17mers_human_m_1MM_117_p.txt | Salmonella enterica serovar Typhi |
| | | (Salmonella typhi) strain CT18 plasmid pHCM1 |
| 119. | 17mers_human_m_1MM_118_p.txt | Salmonella enterica serovar Typhi |
| | | (Salmonella typhi) strain CT18 plasmid pHCM2 |
| 120. | 17mers_human_m_1MM_119_p.txt | Salmonella typhimurium LT2 |
| 121. | 17mers_human_m_1MM_120_p.txt | Salmonella typhimurium LT2 strain |
| | | SGSC1412 plasmid pSLT complete |
| 122. | 17mers_human_m_1MM_121_p.txt | Shewanella oneidensis MR-1 complete |
| | | genome. |
| 123. | 17mers_human_m_1MM_122_p.txt | Shewanella oneidensis MR-1 |
| | | megaplasmid complete sequence. |
| 124. | 17mers_human_m_1MM_123_p.txt | Shigella flexneri 2a str. 301 complete |
| | | genome. |
| 125. | 17mers_human_m_1MM_124_p.txt | Sinorhizobium meliloti 1021 complete |
| | | genomes. |
| 126. | 17mers_human_m_1MM_125_p.txt | Sinorhizobium meliloti plasmid pSymA |
| | | complete plasmid sequence. |
| 127. | 17mers_human_m_1MM_126_p.txt | Sinorhizobium meliloti plasmid pSym B |
| 128. | 17mers_human_m_1MM_127_p.txt | Staphylococcus aureus MW2 |
| 129. | 17mers_human_m_1MM_128_p.txt | Staphylococcus aureus strain Mu50 |
| 130. | 17mers_human_m_1MM_129_p.txt | Staphylococcus aureus plasmid VRSAp |
| 131. | 17mers_human_m_1MM_130_p.txt | Staphylococcus aureus strain N315 |
| 132. | 17mers_human_m_1MM_131_p.txt | Staphylococcus aureus subsp. aureus |
| | | N315 plasmid pN315B DNA |
| 133. | 17mers_human_m_1MM_132_p.txt | Streptococcus agalactiae |
| 134. | 17mers_human_m_1MM_133_p.txt | Streptococcus agalactiae NEM316 |
| 135. | 17mers_human_m_1MM_134_p.txt | Streptococcus mutans UA 159 complete |
| | | genome. |
| 136. | 17mers_human_m_1MM_135_p.txt | Streptococcus pneumoniae complete |
| | | genome. |
| 137. | 17mers_human_m_1MM_136_p.txt | Streptococcus pneumoniae R6 complete |
| | | genome. |
| 138. | 17mers_human_m_1MM_137_p.txt | Streptococcus pyogenes MGAS315 |
| 139. | 17mers_human_m_1MM_138_p.txt | Streptococcus pyogenes strain |
| | | MGAS8232 |
| 140. | 17mers_human_m_1MM_139_p.txt | Streptococcus pyogenes strain SF370 |
| | | serotype M1 |
| 141. | 17mers_human_m_1MM_140_p.txt | Streptomyces coelicolor A3(2) |
| 142. | | |

CD6 contains Two folders - SEQ ID NO 13: “Microbial 17 mers 1MM” and
SEQ ID NO 14: “Microchip”. The contents of the folder are listed below.
1) SEQ ID NO 13: Folder 17 mers 1MM - It contains 40 files, containing
sequences of length 17, which are present in the microbial genome, but are not
present in the human genome. All sequences produced from these sequences with
one mismatch are also not present in the human genome. There are 180 files in total
and this CD contains the last 40 of them. The list below contains the file name and
the corresponding microbial name.

| | File Name | Species Name |
|---|---|---|
| 143. | 17mers_human_m_1MM_141_p.txt | Streptomyces coelicolor plasmid SCP1. |
| 144. | 17mers_human_m_1MM_142_p.txt | Streptomyces coelicolor plasmid SCP2. |
| 145. | 17mers_human_m_1MM_143_p.txt | Sulfolobus solfataricus complete |
| | | genome. |
| 146. | 17mers_human_m_1MM_144_p.txt | Sulfolobus tokodaii complete genome. |
| 147. | 17mers_human_m_1MM_145_p.txt | Synechocystis PCC6803 complete |
| | | genome. |
| 148. | 17mers_human_m_1MM_146_p.txt | Thermoanaerobacter tengcongensis |
| | | strain MB4T |
| 149. | 17mers_human_m_1MM_147_p.txt | Thermoplasma acidophilum |
| 150. | 17mers_human_m_1MM_148_p.txt | Thermoplasma volcanium |
| 151. | 17mers_human_m_1MM_149_p.txt | Thermosynechococcus elongatus BP-1 |
| 152. | 17mers_human_m_1MM_150_p.txt | Thermotoga maritima complete |
| | | genome. |
| 153. | 17mers_human_m_1MM_151_p.txt | Treponema pallidum complete genome. |
| 154. | 17mers_human_m_1MM_152_p.txt | Ureaplasma urealyticum complete |
| | | genome. |
| 155. | 17mers_human_m_1MM_153_p.txt | Vibrio cholerae chromosome I |
| 156. | 17mers_human_m_1MM_154_p.txt | Vibrio vulnificus CMCP6 chromosome |
| | | I complete sequence. |
| 157. | 17mers_human_m_1MM_156_p.txt | Wigglesworthia brevipalpis |
| 158. | 17mers_human_m_1MM_157_p.txt | Xanthomonas axonopodis pv. citri str. |
| | | 306 |
| 159. | 17mers_human_m_1MM_159_p.txt | Xanthomonas campestris pv. campestris |
| | | str. ATCC 33913 |
| 160. | 17mers_human_m_1MM_160_p.txt | Xylella fastidiosa |
| 161. | 17mers_human_m_1MM_161_p.txt | Xylella fastidiosa plasmid pXF1.3 |
| 162. | 17mers_human_m_1MM_162_p.txt | Xylella fastidiosa plasmid pXF51 |
| 163. | 17mers_human_m_1MM_163_p.txt | Xylella fastidiosa plasmid pXF868 |
| 164. | 17mers_human_m_1MM_164_p.txt | Yersinia pestis KIM complete genome. |
| 165. | 17mers_human_m_1MM_165_p.txt | Yersinia pestis strain CO92 |
| 166. | 17mers_human_m_1MM_166_p.txt | Yersinia pestis plasmid pCD1. |
| 167. | 17mers_human_m_1MM_167_p.txt | Yersinia pestis plasmid pPCP1. |
| 168. | 17mers_human_m_1MM_168_p.txt | Yersinia pestis plasmid pPMT1. |
| 169. | 17mers_human_m_1MM_169_p.txt | Chlamydophila pneumoniae CWL029 |
| | | complete genome |
| 170. | 17mers_human_m_1MM_170_p.txt | Nitrosomonas europaea |
| 171. | 17mers_human_m_1MM_171_p.txt | Prochlorococcus marinus MED4 |
| 172. | 17mers_human_m_1MM_172_p.txt | Prochlorococcus marinus MIT9313 |
| 173. | 17mers_human_m_1MM_173_p.txt | Rhodopseudomonas palustris |
| 174. | 17mers_human_m_1MM_174_p.txt | Staphylococcus epidermidis ATCC |
| | | 12228, complete genome |
| 175. | 17mers_human_m_1MM_175_p.txt | Synechococcus |
| 176. | 17mers_human_m_1MM_176_p.txt | Clostridium tetani E88, complete |
| | | genome. |
| 177. | 17mers_human_m_1MM_177_p.txt | Lactobacillus plantarum WCFS 1, |
| | | complete genome. |
| 178. | 17mers_human_m_1MM_178_p.txt | Pseudomonas syringae pv. tomato str. |
| | | DC3000 |
| 179. | 17mers_human_m_1MM_179_p.txt | Streptococcus pyogenes phage 315.3 |
| | | provirus |
| 180. | 17mers_human_m_1MM_180_p.txt | Tropheryma whipplei str. Twist |
| 181. | 17mers_human_m_1MM_181_p.txt | Vibrio parahaemolyticus RIMD |
| | | 2210633, chromosome 1, complete sequence |
| 182. | 17mers_human_m_1MM_182_p.txt | Xylella fastidiosa Temeculal |

2) SEQ ID NO 14: Microchip - This folder contains 8 files containing sequences, of length 18,
which are present in a particular microbial and not present in any other tested microbial or human
genome even with any two changes. To make the melting temperature of all probes similar, the
GC content of each of these sequences is equal 10.
This is an example of how the Microchip can be designed for any organism.
CD 7 contains 3 files:

| | File Name | Species Name |
|---|---|---|
| 1. | microbial_2_65.txt | Agrobacterium tumefaciens strain C58 circular chromosome |
| 2. | microbial_2_142.txt | Bacillus subtilis complete genome. |
| 3. | microbial_2_90.txt | Campylobacter jejuni complete genome. |
| 4. | microbial_2_146.txt | Corynebacterium glutamicum ATCC 13032 |
| 5. | microbial_2_139.txt | Escherichia coli K-12 MG1655 complete genome. |
| 6. | microbial_2_23.txt | Helicobacter pylori |
| 7. | microbial_2_46.txt | Pseudomonas aeruginosa PA01 |
| 8. | microbial_2_149.txt | Pseudomonas putida KT2440 |

CD7 contains three folders - SEQ ID NO 15 “nmerseqsAllFlavi”, SEQ ID
NO 16 “nmerseqsDV” and SEQ ID NO 17 “nmerseqsWNV”.
Folder nmerseqsAllFlavi - It contains 765 Flavivirus human-blind files, containing
sequences which are present in Flavivirus (other than Dengue and West Nile) genomes
but are not present in Human genome even with two mismatches. The list below gives us
the file name and the corresponding virus name.

| | | |
|---|---|---|
| 1) | Alkhurma(NC_004355)_16mers_0MM.txt | Alkhurma (NC_004355) |
| 2) | Alkhurma(NC_004355)_16mers_1MM.txt | Alkhurma (NC_004355) |
| 3) | Alkhurma(NC_004355)_16mers_2MM.txt | Alkhurma (NC_004355) |
| 4) | Alkhurma(NC_004355)_17mers_0MM.txt | Alkhurma (NC_004355) |
| 5) | Alkhurma(NC_004355)_17mers_1MM.txt | Alkhurma (NC_004355) |
| 6) | Alkhurma(NC_004355)_17mers_2MM.txt | Alkhurma (NC_004355) |
| 7) | Alkhurma(NC_004355)_18mers_0MM.txt | Alkhurma (NC_004355) |
| 8) | Alkhurma(NC_004355)_18mers_1MM.txt | Alkhurma (NC_004355) |
| 9) | Alkhurma(NC_004355)_18mers_2MM.txt | Alkhurma (NC_004355) |
| 10) | Apoi(NC_003676)_16mers_0MM.txt | Apoi (NC_003676) |
| 11) | Apoi(NC_003676)_16mers_1MM.txt | Apoi (NC_003676) |
| 12) | Apoi(NC_003676)_16mers_2MM.txt | Apoi (NC_003676) |
| 13) | Apoi(NC_003676)_17mers_0MM.txt | Apoi (NC_003676) |
| 14) | Apoi(NC_003676)_17mers_1MM.txt | Apoi (NC_003676) |
| 15) | Apoi(NC_003676)_17mers_2MM.txt | Apoi (NC_003676) |
| 16) | Apoi(NC_003676)_18mers_0MM.txt | Apoi (NC_003676) |
| 17) | Apoi(NC_003676)_18mers_1MM.txt | Apoi (NC_003676) |
| 18) | Apoi(NC_003676)_18mers_2MM.txt | Apoi (NC_003676) |
| 19) | Cell_fusing_agent_virus(NC_001564)_16mers_0MM.txt | Cell fusing agent virus (NC_001564) |
| 20) | Cell_fusing_agent_virus(NC_001564)_16mers_1MM.txt | Cell fusing agent virus (NC_001564) |
| 21) | Cell_fusing_agent_virus(NC_001564)_16mers_2MM.txt | Cell fusing agent virus (NC_001564) |
| 22) | Cell_fusing_agent_virus(NC_001564)_17mers_0MM.txt | Cell fusing agent virus (NC_001564) |
| 23) | Cell_fusing_agent_virus(NC_001564)_17mers_1MM.txt | Cell fusing agent virus (NC_001564) |
| 24) | Cell_fusing_agent_virus(NC_001564)_17mers_2MM.txt | Cell fusing agent virus (NC_001564) |
| 25) | Cell_fusing_agent_virus(NC_001564)_18mers_0MM.txt | Cell fusing agent virus (NC_001564) |
| 26) | Cell_fusing_agent_virus(NC_001564)_18mers_1MM.txt | Cell fusing agent virus (NC_001564) |
| 27) | Cell_fusing_agent_virus(NC_001564)_18mers_2MM.txt | Cell fusing agent virus (NC_001564) |
| 28) | Deer_tick(NC_003218)_16mers_0MM.txt | Deer tick (NC_003218) |
| 29) | Deer_tick(NC_003218)_16mers_1MM.txt | Deer tick (NC_003218) |
| 30) | Deer_tick(NC_003218)_16mers_2MM.txt | Deer tick (NC_003218) |
| 31) | Deer_tick(NC_003218)_17mers_0MM.txt | Deer tick (NC_003218) |
| 32) | Deer_tick(NC_003218)_17mers_1MM.txt | Deer tick (NC_003218) |
| 33) | Deer_tick(NC_003218)_17mers_2MM.txt | Deer tick (NC_003218) |
| 34) | Deer_tick(NC_003218)_18mers_0MM.txt | Deer tick (NC_003218) |
| 35) | Deer_tick(NC_003218)_18mers_1MM.txt | Deer tick (NC_003218) |
| 36) | Deer_tick(NC_003218)_18mers_2MM.txt | Deer tick (NC_003218) |
| 37) | Japanese_encephalitis(NC_001437)_16mers_0MM.txt | Japanese encephalitis (NC_001437) |
| 38) | Japanese_encephalitis(NC_001437)_16mers_1MM.txt | Japanese encephalitis (NC_001437) |
| 39) | Japanese_encephalitis(NC_001437)_16mers_2MM.txt | Japanese encephalitis (NC_001437) |
| 40) | Japanese_encephalitis(NC_001437)_17mers_0MM.txt | Japanese encephalitis (NC_001437) |
| 41) | Japanese_encephalitis(NC_001437)_17mers_1MM.txt | Japanese encephalitis (NC_001437) |
| 42) | Japanese_encephalitis(NC_001437)_17mers_2MM.txt | Japanese encephalitis (NC_001437) |
| 43) | Japanese_encephalitis(NC_001437)_18mers_0MM.txt | Japanese encephalitis (NC_001437) |
| 44) | Japanese_encephalitis(NC_001437)_18mers_1MM.txt | Japanese encephalitis (NC_001437) |
| 45) | Japanese_encephalitis(NC_001437)_18mers_2MM.txt | Japanese encephalitis (NC_001437) |
| 46) | JEV_attenuated_strain_SA14-12-1-7(AF416457)_16mers_0MM.txt | JEV attenuated strain |
| | | SA14-12-1-7(AF416457) |
| 47) | JEV_attenuated_strain_SA14-12-1-7(AF416457)_16mers_1MM.txt | JEV attenuated strain |
| | | SA14-12-1-7(AF416457) |
| 48) | JEV_attenuated_strain_SA14-12-1-7(AF416457)_16mers_2MM.txt | JEV attenuated strain |
| | | SA14-12-1-7(AF416457) |
| 49) | JEV_attenuated_strain_SA14-12-1-7(AF416457)_17mers_0MM.txt | JEV attenuated strain |
| | | SA14-12-1-7(AF416457) |
| 50) | JEV_attenuated_strain_SA14-12-1-7(AF416457)_17mers_1MM.txt | JEV attenuated strain |
| | | SA14-12-1-7(AF416457) |
| 51) | JEV_attenuated_strain_SA14-12-1-7(AF416457)_17mers_2MM.txt | JEV attenuated strain |
| | | SA14-12-1-7(AF416457) |
| 52) | JEV_attenuated_strain_SA14-12-1-7(AF416457)_18mers_0MM.txt | JEV attenuated strain |
| | | SA14-12-1-7(AF416457) |
| 53) | JEV_attenuated_strain_SA14-12-1-7(AF416457)_18mers_1MM.txt | JEV attenuated strain |
| | | SA14-12-1-7(AF416457) |
| 54) | JEV_attenuated_strain_SA14-12-1-7(AF416457)_18mers_2MM.txt | JEV attenuated strain |
| | | SA14-12-1-7(AF416457) |
| 55) | JEV_CNU-LP2(AY585243)_16mers_0MM.txt | JEV CNU-LP2(AY585243) |
| 56) | JEV_CNU-LP2(AY585243)_16mers_1MM.txt | JEV CNU-LP2(AY585243) |
| 57) | JEV_CNU-LP2(AY585243)_16mers_2MM.txt | JEV CNU-LP2(AY585243) |
| 58) | JEV_CNU-LP2(AY585243)_17mers_0MM.txt | JEV CNU-LP2(AY585243) |
| 59) | JEV_CNU-LP2(AY585243)_17mers_1MM.txt | JEV CNU-LP2(AY585243) |
| 60) | JEV_CNU-LP2(AY585243)_17mers_2MM.txt | JEV CNU-LP2(AY585243) |
| 61) | JEV_CNU-LP2(AY585243)_18mers_0MM.txt | JEV CNU-LP2(AY585243) |
| 62) | JEV_CNU-LP2(AY585243)_18mers_1MM.txt | JEV CNU-LP2(AY585243) |
| 63) | JEV_CNU-LP2(AY585243)_18mers_2MM.txt | JEV CNU-LP2(AY585243) |
| 64) | JEV_FU_strain(AF217620)_16mers_0MM.txt | JEV FU strain(AF217620) |
| 65) | JEV_FU_strain(AF217620)_16mers_1MM.txt | JEV FU strain(AF217620) |
| 66) | JEV_FU_strain(AF217620)_16mers_2MM.txt | JEV FU strain(AF217620) |
| 67) | JEV_FU_strain(AF217620)_17mers_0MM.txt | JEV FU strain(AF217620) |
| 68) | JEV_FU_strain(AF217620)_17mers_1MM.txt | JEV FU strain(AF217620) |
| 69) | JEV_FU_strain(AF217620)_17mers_2MM.txt | JEV FU strain(AF217620) |
| 70) | JEV_FU_strain(AF217620)_18mers_0MM.txt | JEV FU strain(AF217620) |
| 71) | JEV_FU_strain(AF217620)_18mers_1MM.txt | JEV FU strain(AF217620) |
| 72) | JEV_FU_strain(AF217620)_18mers_2MM.txt | JEV FU strain(AF217620) |
| 73) | JEV_gene_for_viral_protein(AB051292)_16mers_0MM.txt | JEV gene for viral |
| | | protein(AB051292) |
| 74) | JEV_gene_for_viral_protein(AB051292)_16mers_1MM.txt | JEV gene for viral |
| | | protein(AB051292) |
| 75) | JEV_gene_for_viral_protein(AB051292)_16mers_2MM.txt | JEV gene for viral |
| | | protein(AB051292) |
| 76) | JEV_gene_for_viral_protein(AB051292)_17mers_0MM.txt | JEV gene for viral |
| | | protein(AB051292) |
| 77) | JEV_gene_for_viral_protein(AB051292)_17mers_1MM.txt | JEV gene for viral |
| | | protein(AB051292) |
| 78) | JEV_gene_for_viral_protein(AB051292)_17mers_2MM.txt | JEV gene for viral |
| | | protein(AB051292) |
| 79) | JEV_gene_for_viral_protein(AB051292)_18mers_0MM.txt | JEV gene for viral |
| | | protein(AB051292) |
| 80) | JEV_gene_for_viral_protein(AB051292)_18mers_1MM.txt | JEV gene for viral |
| | | protein(AB051292) |
| 81) | JEV_gene_for_viral_protein(AB051292)_18mers_2MM.txt | JEV gene for viral |
| | | protein(AB051292) |
| 82) | JEV_isolate_CH195LA(AF221499)_16mers_0MM.txt | JEV isolate CH195LA(AF221499) |
| 83) | JEV_isolate_CH195LA(AF221499)_16mers_1MM.txt | JEV isolate CH195LA(AF221499) |
| 84) | JEV_isolate_CH195LA(AF221499)_16mers_2MM.txt | JEV isolate CH195LA(AF221499) |
| 85) | JEV_isolate_CH195LA(AF221499)_17mers_0MM.txt | JEV isolate CH195LA(AF221499) |
| 86) | JEV_isolate_CH195LA(AF221499)_17mers_1MM.txt | JEV isolate CH195LA(AF221499) |
| 87) | JEV_isolate_CH195LA(AF221499)_17mers_2MM.txt | JEV isolate CH195LA(AF221499) |
| 88) | JEV_isolate_CH195LA(AF221499)_18mers_0MM.txt | JEV isolate CH195LA(AF221499) |
| 89) | JEV_isolate_CH195LA(AF221499)_18mers_1MM.txt | JEV isolate CH195LA(AF221499) |
| 90) | JEV_isolate_CH195LA(AF221499)_18mers_2MM.txt | JEV isolate CH195LA(AF221499) |
| 91) | JEV_isolate_CH2195SA(AF221500)_16mers_0MM.txt | JEV isolate CH2195SA(AF221500) |
| 92) | JEV_isolate_CH2195SA(AF221500)_16mers_1MM.txt | JEV isolate CH2195SA(AF221500) |
| 93) | JEV_isolate_CH2195SA(AF221500)_16mers_2MM.txt | JEV isolate CH2195SA(AF221500) |
| 94) | JEV_isolate_CH2195SA(AF221500)_17mers_0MM.txt | JEV isolate CH2195SA(AF221500) |
| 95) | JEV_isolate_CH2195SA(AF221500)_17mers_1MM.txt | JEV isolate CH2195SA(AF221500) |
| 96) | JEV_isolate_CH2195SA(AF221500)_17mers_2MM.txt | JEV isolate CH2195SA(AF221500) |
| 97) | JEV_isolate_CH2195SA(AF221500)_18mers_0MM.txt | JEV isolate CH2195SA(AF221500) |
| 98) | JEV_isolate_CH2195SA(AF221500)_18mers_1MM.txt | JEV isolate CH2195SA(AF221500) |
| 99) | JEV_isolate_CH2195SA(AF221500)_18mers_2MM.txt | JEV isolate CH2195SA(AF221500) |
| 100) | JEV_isolate_Vellore_P20778(AF080251)_16mers_0MM.txt | JEV isolate Vellore |
| | | P20778(AF080251) |
| 101) | JEV_isolate_Vellore_P20778(AF080251)_16mers_1MM.txt | JEV isolate Vellore |
| | | P20778(AF080251) |
| 102) | JEV_isolate_Vellore_P20778(AF080251)_16mers_2MM.txt | JEV isolate Vellore |
| | | P20778(AF080251) |
| 103) | JEV_isolate_Vellore_P20778(AF080251)_17mers_0MM.txt | JEV isolate Vellore |
| | | P20778(AF080251) |
| 104) | JEV_isolate_Vellore_P20778(AF080251)_17mers_1MM.txt | JEV isolate Vellore |
| | | P20778(AF080251) |
| 105) | JEV_isolate_Vellore_P20778(AF080251)_17mers_2MM.txt | JEV isolate Vellore |
| | | P20778(AF080251) |
| 106) | JEV_isolate_Vellore_P20778(AF080251)_18mers_0MM.txt | JEV isolate Vellore |
| | | P20778(AF080251) |
| 107) | JEV_isolate_Vellore_P20778(AF080251)_18mers_1MM.txt | JEV isolate Vellore |
| | | P20778(AF080251) |
| 108) | JEV_isolate_Vellore_P20778(AF080251)_18mers_2MM.txt | JEV isolate Vellore |
| | | P20778(AF080251) |
| 109) | JEV_JaGAr_01(AF069076)_16mers_0MM.txt | JEV JaGAr 01(AF069076) |
| 110) | JEV_JaGAr_01(AF069076)_16mers_1MM.txt | JEV JaGAr 01(AF069076) |
| 111) | JEV_JaGAr_01(AF069076)_16mers_2MM.txt | JEV JaGAr 01(AF069076) |
| 112) | JEV_JaGAr_01(AF069076)_17mers_0MM.txt | JEV JaGAr 01(AF069076) |
| 113) | JEV_JaGAr_01(AF069076)_17mers_1MM.txt | JEV JaGAr 01(AF069076) |
| 114) | JEV_JaGAr_01(AF069076)_17mers_2MM.txt | JEV JaGAr 01(AF069076) |
| 115) | JEV_JaGAr_01(AF069076)_18mers_0MM.txt | JEV JaGAr 01(AF069076) |
| 116) | JEV_JaGAr_01(AF069076)_18mers_1MM.txt | JEV JaGAr 01(AF069076) |
| 117) | JEV_JaGAr_01(AF069076)_18mers_2MM.txt | JEV JaGAr 01(AF069076) |
| 118) | JEV_polyprotein_gene(AF014160)_16mers_0MM.txt | JEV polyprotein gene(AF014160) |
| 119) | JEV_polyprotein_gene(AF014160)_16mers_1MM.txt | JEV polyprotein gene(AF014160) |
| 120) | JEV_polyprotein_gene(AF014160)_16mers_2MM.txt | JEV polyprotein gene(AF014160) |
| 121) | JEV_polyprotein_gene(AF014160)_17mers_0MM.txt | JEV polyprotein gene(AF014160) |
| 122) | JEV_polyprotein_gene(AF014160)_17mers_1MM.txt | JEV polyprotein gene(AF014160) |
| 123) | JEV_polyprotein_gene(AF014160)_17mers_2MM.txt | JEV polyprotein gene(AF014160) |
| 124) | JEV_polyprotein_gene(AF014160)_18mers_0MM.txt | JEV polyprotein gene(AF014160) |
| 125) | JEV_polyprotein_gene(AF014160)_18mers_1MM.txt | JEV polyprotein gene(AF014160) |
| 126) | JEV_polyprotein_gene(AF014160)_18mers_2MM.txt | JEV polyprotein gene(AF014160) |
| 127) | JEV_polyprotein_gene(AF014161)_16mers_0MM.txt | JEV polyprotein gene(AF014161) |
| 128) | JEV_polyprotein_gene(AF014161)_16mers_1MM.txt | JEV polyprotein gene(AF014161) |
| 129) | JEV_polyprotein_gene(AF014161)_16mers_2MM.txt | JEV polyprotein gene(AF014161) |
| 130) | JEV_polyprotein_gene(AF014161)_17mers_0MM.txt | JEV polyprotein gene(AF014161) |
| 131) | JEV_polyprotein_gene(AF014161)_17mers_1MM.txt | JEV polyprotein gene(AF014161) |
| 132) | JEV_polyprotein_gene(AF014161)_17mers_2MM.txt | JEV polyprotein gene(AF014161) |
| 133) | JEV_polyprotein_gene(AF014161)_18mers_0MM.txt | JEV polyprotein gene(AF014161) |
| 134) | JEV_polyprotein_gene(AF014161)_18mers_1MM.txt | JEV polyprotein gene(AF014161) |
| 135) | JEV_polyprotein_gene(AF014161)_18mers_2MM.txt | JEV polyprotein gene(AF014161) |
| 136) | JEV_polyprotein_mRNA(U15763)_16mers_0MM.txt | JEV polyprotein mRNA(U15763) |
| 137) | JEV_polyprotein_mRNA(U15763)_16mers_1MM.txt | JEV polyprotein mRNA(U15763) |
| 138) | JEV_polyprotein_mRNA(U15763)_16mers_2MM.txt | JEV polyprotein mRNA(U15763) |
| 139) | JEV_polyprotein_mRNA(U15763)_17mers_0MM.txt | JEV polyprotein mRNA(U15763) |
| 140) | JEV_polyprotein_mRNA(U15763)_17mers_1MM.txt | JEV polyprotein mRNA(U15763) |
| 141) | JEV_polyprotein_mRNA(U15763)_17mers_2MM.txt | JEV polyprotein mRNA(U15763) |
| 142) | JEV_polyprotein_mRNA(U15763)_18mers_0MM.txt | JEV polyprotein mRNA(U15763) |
| 143) | JEV_polyprotein_mRNA(U15763)_18mers_1MM.txt | JEV polyprotein mRNA(U15763) |
| 144) | JEV_polyprotein_mRNA(U15763)_18mers_2MM.txt | JEV polyprotein mRNA(U15763) |
| 145) | JEV_SA-14(M55506)_16mers_0MM.txt | JEV SA-14(M55506) |
| 146) | JEV_SA-14(M55506)_16mers_1MM.txt | JEV SA-14(M55506) |
| 147) | JEV_SA-14(M55506)_16mers_2MM.txt | JEV SA-14(M55506) |
| 148) | JEV_SA-14(M55506)_17mers_0MM.txt | JEV SA-14(M55506) |
| 149) | JEV_SA-14(M55506)_17mers_1MM.txt | JEV SA-14(M55506) |
| 150) | JEV_SA-14(M55506)_17mers_2MM.txt | JEV SA-14(M55506) |
| 151) | JEV_SA-14(M55506)_18mers_0MM.txt | JEV SA-14(M55506) |
| 152) | JEV_SA-14(M55506)_18mers_1MM.txt | JEV SA-14(M55506) |
| 153) | JEV_SA-14(M55506)_18mers_2MM.txt | JEV SA-14(M55506) |
| 154) | JEV_SA14_polyprotein_mRNA(U14163)_16mers_0MM.txt | JEV SA14 polyprotein |
| | | mRNA(U14163) |
| 155) | JEV_SA14_polyprotein_mRNA(U14163)_16mers_1MM.txt | JEV SA14 polyprotein |
| | | mRNA(U14163) |
| 156) | JEV_SA14_polyprotein_mRNA(U14163)_16mers_2MM.txt | JEV SA14 polyprotein |
| | | mRNA(U14163) |
| 157) | JEV_SA14_polyprotein_mRNA(U14163)_17mers_0MM.txt | JEV SA14 polyprotein |
| | | mRNA(U14163) |
| 158) | JEV_SA14_polyprotein_mRNA(U14163)_17mers_1MM.txt | JEV SA14 polyprotein |
| | | mRNA(U14163) |
| 159) | JEV_SA14_polyprotein_mRNA(U14163)_17mers_2MM.txt | JEV SA14 polyprotein |
| | | mRNA(U14163) |
| 160) | JEV_SA14_polyprotein_mRNA(U14163)_18mers_0MM.txt | JEV SA14 polyprotein |
| | | mRNA(U14163) |
| 161) | JEV_SA14_polyprotein_mRNA(U14163)_18mers_1MM.txt | JEV SA14 polyprotein |
| | | mRNA(U14163) |
| 162) | JEV_SA14_polyprotein_mRNA(U14163)_18mers_2MM.txt | JEV SA14 polyprotein |
| | | mRNA(U14163) |
| 163) | JEV_strain_Beijing-1(L48961)_16mers_0MM.txt | JEV strain Beijing-1(L48961) |
| 164) | JEV_strain_Beijing-1(L48961)_16mers_1MM.txt | JEV strain Beijing-1(L48961) |
| 165) | JEV_strain_Beijing-1(L48961)_16mers_2MM.txt | JEV strain Beijing-1(L48961) |
| 166) | JEV_strain_Beijing-1(L48961)_17mers_0MM.txt | JEV strain Beijing-1(L48961) |
| 167) | JEV_strain_Beijing-1(L48961)_17mers_1MM.txt | JEV strain Beijing-1(L48961) |
| 168) | JEV_strain_Beijing-1(L48961)_17mers_2MM.txt | JEV strain Beijing-1(L48961) |
| 169) | JEV_strain_Beijing-1(L48961)_18mers_0MM.txt | JEV strain Beijing-1(L48961) |
| 170) | JEV_strain_Beijing-1(L48961)_18mers_1MM.txt | JEV strain Beijing-1(L48961) |
| 171) | JEV_strain_Beijing-1(L48961)_18mers_2MM.txt | JEV strain Beijing-1(L48961) |
| 172) | JEV_strain_CC27-L1(AY303795)_16mers_0MM.txt | JEV strain CC27-L1(AY303795) |
| 173) | JEV_strain_CC27-L1(AY303795)_16mers_1MM.txt | JEV strain CC27-L1(AY303795) |
| 174) | JEV_strain_CC27-L1(AY303795)_16mers_2MM.txt | JEV strain CC27-L1(AY303795) |
| 175) | JEV_strain_CC27-L1(AY303795)_17mers_0MM.txt | JEV strain CC27-L1(AY303795) |
| 176) | JEV_strain_CC27-L1(AY303795)_17mers_1MM.txt | JEV strain CC27-L1(AY303795) |
| 177) | JEV_strain_CC27-L1(AY303795)_17mers_2MM.txt | JEV strain CC27-L1(AY303795) |
| 178) | JEV_strain_CC27-L1(AY303795)_18mers_0MM.txt | JEV strain CC27-L1(AY303795) |
| 179) | JEV_strain_CC27-L1(AY303795)_18mers_1MM.txt | JEV strain CC27-L1(AY303795) |
| 180) | JEV_strain_CC27-L1(AY303795)_18mers_2MM.txt | JEV strain CC27-L1(AY303795) |
| 181) | JEV_strain_CC27-L3(AY303796)_16mers_0MM.txt | JEV strain CC27-L3(AY303796) |
| 182) | JEV_strain_CC27-L3(AY303796)_16mers_1MM.txt | JEV strain CC27-L3(AY303796) |
| 183) | JEV_strain_CC27-L3(AY303796)_16mers_2MM.txt | JEV strain CC27-L3(AY303796) |
| 184) | JEV_strain_CC27-L3(AY303796)_17mers_0MM.txt | JEV strain CC27-L3(AY303796) |
| 185) | JEV_strain_CC27-L3(AY303796)_17mers_1MM.txt | JEV strain CC27-L3(AY303796) |
| 186) | JEV_strain_CC27-L3(AY303796)_17mers_2MM.txt | JEV strain CC27-L3(AY303796) |
| 187) | JEV_strain_CC27-L3(AY303796)_18mers_0MM.txt | JEV strain CC27-L3(AY303796) |
| 188) | JEV_strain_CC27-L3(AY303796)_18mers_1MM.txt | JEV strain CC27-L3(AY303796) |
| 189) | JEV_strain_CC27-L3(AY303796)_18mers_2MM.txt | JEV strain CC27-L3(AY303796) |
| 190) | JEV_strain_CC27-S6(AY303797)_16mers_0MM.txt | JEV strain CC27-S6(AY303797) |
| 191) | JEV_strain_CC27-S6(AY303797)_16mers_1MM.txt | JEV strain CC27-S6(AY303797) |
| 192) | JEV_strain_CC27-S6(AY303797)_16mers_2MM.txt | JEV strain CC27-S6(AY303797) |
| 193) | JEV_strain_CC27-S6(AY303797)_17mers_0MM.txt | JEV strain CC27-S6(AY303797) |
| 194) | JEV_strain_CC27-S6(AY303797)_17mers_1MM.txt | JEV strain CC27-S6(AY303797) |
| 195) | JEV_strain_CC27-S6(AY303797)_17mers_2MM.txt | JEV strain CC27-S6(AY303797) |
| 196) | JEV_strain_CC27-S6(AY303797)_18mers_0MM.txt | JEV strain CC27-S6(AY303797) |
| 197) | JEV_strain_CC27-S6(AY303797)_18mers_1MM.txt | JEV strain CC27-S6(AY303797) |
| 198) | JEV_strain_CC27-S6(AY303797)_18mers_2MM.txt | JEV strain CC27-S6(AY303797) |
| 199) | JEV_strain_CH1392(AF254452)_16mers_0MM.txt | JEV strain CH1392(AF254452) |
| 200) | JEV_strain_CH1392(AF254452)_16mers_1MM.txt | JEV strain CH1392(AF254452) |
| 201) | JEV_strain_CH1392(AF254452)_16mers_2MM.txt | JEV strain CH1392(AF254452) |
| 202) | JEV_strain_CH1392(AF254452)_17mers_0MM.txt | JEV strain CH1392(AF254452) |
| 203) | JEV_strain_CH1392(AF254452)_17mers_1MM.txt | JEV strain CH1392(AF254452) |
| 204) | JEV_strain_CH1392(AF254452)_17mers_2MM.txt | JEV strain CH1392(AF254452) |
| 205) | JEV_strain_CH1392(AF254452)_18mers_0MM.txt | JEV strain CH1392(AF254452) |
| 206) | JEV_strain_CH1392(AF254452)_18mers_1MM.txt | JEV strain CH1392(AF254452) |
| 207) | JEV_strain_CH1392(AF254452)_18mers_2MM.txt | JEV strain CH1392(AF254452) |
| 208) | JEV_strain_CJN-L1(AY202794)_16mers_0MM.txt | JEV strain CJN-L1(AY202794) |
| 209) | JEV_strain_CJN-L1(AY202794)_16mers_1MM.txt | JEV strain CJN-L1(AY202794) |
| 210) | JEV_strain_CJN-L1(AY202794)_16mers_2MM.txt | JEV strain CJN-L1(AY202794) |
| 211) | JEV_strain_CJN-L1(AY202794)_17mers_0MM.txt | JEV strain CJN-L1(AY202794) |
| 212) | JEV_strain_CJN-L1(AY202794)_17mers_1MM.txt | JEV strain CJN-L1(AY202794) |
| 213) | JEV_strain_CJN-L1(AY202794)_17mers_2MM.txt | JEV strain CJN-L1(AY202794) |
| 214) | JEV_strain_CJN-L1(AY202794)_18mers_0MM.txt | JEV strain CJN-L1(AY202794) |
| 215) | JEV_strain_CJN-L1(AY202794)_18mers_1MM.txt | JEV strain CJN-L1(AY202794) |
| 216) | JEV_strain_CJN-L1(AY202794)_18mers_2MM.txt | JEV strain CJN-L1(AY202794) |
| 217) | JEV_strain_CJN-S1(AY303793)_16mers_0MM.txt | JEV strain CJN-S1(AY303793) |
| 218) | JEV_strain_CJN-S1(AY303793)_16mers_1MM.txt | JEV strain CJN-S1(AY303793) |
| 219) | JEV_strain_CJN-S1(AY303793)_16mers_2MM.txt | JEV strain CJN-S1(AY303793) |
| 220) | JEV_strain_CJN-S1(AY303793)_17mers_0MM.txt | JEV strain CJN-S1(AY303793) |
| 221) | JEV_strain_CJN-S1(AY303793)_17mers_1MM.txt | JEV strain CJN-S1(AY303793) |
| 222) | JEV_strain_CJN-S1(AY303793)_17mers_2MM.txt | JEV strain CJN-S1(AY303793) |
| 223) | JEV_strain_CJN-S1(AY303793)_18mers_0MM.txt | JEV strain CJN-S1(AY303793) |
| 224) | JEV_strain_CJN-S1(AY303793)_18mers_1MM.txt | JEV strain CJN-S1(AY303793) |
| 225) | JEV_strain_CJN-S1(AY303793)_18mers_2MM.txt | JEV strain CJN-S1(AY303793) |
| 226) | JEV_strain_CJN-S8(AY303798)_16mers_0MM.txt | JEV strain CJN-S8(AY303798) |
| 227) | JEV_strain_CJN-S8(AY303798)_16mers_1MM.txt | JEV strain CJN-S8(AY303798) |
| 228) | JEV_strain_CJN-S8(AY303798)_16mers_2MM.txt | JEV strain CJN-S8(AY303798) |
| 229) | JEV_strain_CJN-S8(AY303798)_17mers_0MM.txt | JEV strain CJN-S8(AY303798) |
| 230) | JEV_strain_CJN-S8(AY303798)_17mers_1MM.txt | JEV strain CJN-S8(AY303798) |
| 231) | JEV_strain_CJN-S8(AY303798)_17mers_2MM.txt | JEV strain CJN-S8(AY303798) |
| 232) | JEV_strain_CJN-S8(AY303798)_18mers_0MM.txt | JEV strain CJN-S8(AY303798) |
| 233) | JEV_strain_CJN-S8(AY303798)_18mers_1MM.txt | JEV strain CJN-S8(AY303798) |
| 234) | JEV_strain_CJN-S8(AY303798)_18mers_2MM.txt | JEV strain CJN-S8(AY303798) |
| 235) | JEV_strain_GP78(AF075723)_16mers_0MM.txt | JEV strain GP78(AF075723) |
| 236) | JEV_strain_GP78(AF075723)_16mers_1MM.txt | JEV strain GP78(AF075723) |
| 237) | JEV_strain_GP78(AF075723)_16mers_2MM.txt | JEV strain GP78(AF075723) |
| 238) | JEV_strain_GP78(AF075723)_17mers_0MM.txt | JEV strain GP78(AF075723) |
| 239) | JEV_strain_GP78(AF075723)_17mers_1MM.txt | JEV strain GP78(AF075723) |
| 240) | JEV_strain_GP78(AF075723)_17mers_2MM.txt | JEV strain GP78(AF075723) |
| 241) | JEV_strain_GP78(AF075723)_18mers_0MM.txt | JEV strain GP78(AF075723) |
| 242) | JEV_strain_GP78(AF075723)_18mers_1MM.txt | JEV strain GP78(AF075723) |
| 243) | JEV_strain_GP78(AF075723)_18mers_2MM.txt | JEV strain GP78(AF075723) |
| 244) | JEV_strain_HVI(AF098735)_16mers_0MM.txt | JEV strain HVI(AF098735) |
| 245) | JEV_strain_HVI(AF098735)_16mers_1MM.txt | JEV strain HVI(AF098735) |
| 246) | JEV_strain_HVI(AF098735)_16mers_2MM.txt | JEV strain HVI(AF098735) |
| 247) | JEV_strain_HVI(AF098735)_17mers_0MM.txt | JEV strain HVI(AF098735) |
| 248) | JEV_strain_HVI(AF098735)_17mers_1MM.txt | JEV strain HVI(AF098735) |
| 249) | JEV_strain_HVI(AF098735)_17mers_2MM.txt | JEV strain HVI(AF098735) |
| 250) | JEV_strain_HVI(AF098735)_18mers_0MM.txt | JEV strain HVI(AF098735) |
| 251) | JEV_strain_HVI(AF098735)_18mers_1MM.txt | JEV strain HVI(AF098735) |
| 252) | JEV_strain_HVI(AF098735)_18mers_2MM.txt | JEV strain HVI(AF098735) |
| 253) | JEV_strain_JKT6468_polyprotein_gene(AY184212)_16mers_0MM.txt | JEV strain JKT6468 |
| | | polyprotein gene(AY184212) |
| 254) | JEV_strain_JKT6468_polyprotein_gene(AY184212)_16mers_1MM.txt | JEV strain JKT6468 |
| | | polyprotein gene(AY184212) |
| 255) | JEV_strain_JKT6468_polyprotein_gene(AY184212)_16mers_2MM.txt | JEV strain JKT6468 |
| | | polyprotein gene(AY184212) |
| 256) | JEV_strain_JKT6468_polyprotein_gene(AY184212)_17mers_0MM.txt | JEV strain JKT6468 |
| | | polyprotein gene(AY184212) |
| 257) | JEV_strain_JKT6468_polyprotein_gene(AY184212)_17mers_1MM.txt | JEV strain JKT6468 |
| | | polyprotein gene(AY184212) |
| 258) | JEV_strain_JKT6468_polyprotein_gene(AY184212)_17mers_2MM.txt | JEV strain JKT6468 |
| | | polyprotein gene(AY184212) |
| 259) | JEV_strain_JKT6468_polyprotein_gene(AY184212)_18mers_0MM.txt | JEV strain JKT6468 |
| | | polyprotein gene(AY184212) |
| 260) | JEV_strain_JKT6468_polyprotein_gene(AY184212)_18mers_1MM.txt | JEV strain JKT6468 |
| | | polyprotein gene(AY184212) |
| 261) | JEV_strain_JKT6468_polyprotein_gene(AY184212)_18mers_2MM.txt | JEV strain JKT6468 |
| | | polyprotein gene(AY184212) |
| 262) | JEV_strain_K87P39(AY585242)_16mers_0MM.txt | JEV strain K87P39(AY585242) |
| 263) | JEV_strain_K87P39(AY585242)_16mers_1MM.txt | JEV strain K87P39(AY585242) |
| 264) | JEV_strain_K87P39(AY585242)_16mers_2MM.txt | JEV strain K87P39(AY585242) |
| 265) | JEV_strain_K87P39(AY585242)_17mers_0MM.txt | JEV strain K87P39(AY585242) |
| 266) | JEV_strain_K87P39(AY585242)_17mers_1MM.txt | JEV strain K87P39(AY585242) |
| 267) | JEV_strain_K87P39(AY585242)_17mers_2MM.txt | JEV strain K87P39(AY585242) |
| 268) | JEV_strain_K87P39(AY585242)_18mers_0MM.txt | JEV strain K87P39(AY585242) |
| 269) | JEV_strain_K87P39(AY585242)_18mers_1MM.txt | JEV strain K87P39(AY585242) |
| 270) | JEV_strain_K87P39(AY585242)_18mers_2MM.txt | JEV strain K87P39(AY585242) |
| 271) | JEV_strain_K94P05(AF045551)_16mers_0MM.txt | JEV strain K94P05(AF045551) |
| 272) | JEV_strain_K94P05(AF045551)_16mers_1MM.txt | JEV strain K94P05(AF045551) |
| 273) | JEV_strain_K94P05(AF045551)_16mers_2MM.txt | JEV strain K94P05(AF045551) |
| 274) | JEV_strain_K94P05(AF045551)_17mers_0MM.txt | JEV strain K94P05(AF045551) |
| 275) | JEV_strain_K94P05(AF045551)_17mers_1MM.txt | JEV strain K94P05(AF045551) |
| 276) | JEV_strain_K94P05(AF045551)_17mers_2MM.txt | JEV strain K94P05(AF045551) |
| 277) | JEV_strain_K94P05(AF045551)_18mers_0MM.txt | JEV strain K94P05(AF045551) |
| 278) | JEV_strain_K94P05(AF045551)_18mers_1MM.txt | JEV strain K94P05(AF045551) |
| 279) | JEV_strain_K94P05(AF045551)_18mers_2MM.txt | JEV strain K94P05(AF045551) |
| 280) | JEV_strain_KV1899(AY316157)_16mers_0MM.txt | JEV strain KV1899(AY316157) |
| 281) | JEV_strain_KV1899(AY316157)_16mers_1MM.txt | JEV strain KV1899(AY316157) |
| 282) | JEV_strain_KV1899(AY316157)_16mers_2MM.txt | JEV strain KV1899(AY316157) |
| 283) | JEV_strain_KV1899(AY316157)_17mers_0MM.txt | JEV strain KV1899(AY316157) |
| 284) | JEV_strain_KV1899(AY316157)_17mers_1MM.txt | JEV strain KV1899(AY316157) |
| 285) | JEV_strain_KV1899(AY316157)_17mers_2MM.txt | JEV strain KV1899(AY316157) |
| 286) | JEV_strain_KV1899(AY316157)_18mers_0MM.txt | JEV strain KV1899(AY316157) |
| 287) | JEV_strain_KV1899(AY316157)_18mers_1MM.txt | JEV strain KV1899(AY316157) |
| 288) | JEV_strain_KV1899(AY316157)_18mers_2MM.txt | JEV strain KV1899(AY316157) |
| 289) | JEV_strain_Ling(L78128)_16mers_0MM.txt | JEV strain Ling(L78128) |
| 290) | JEV_strain_Ling(L78128)_16mers_1MM.txt | JEV strain Ling(L78128) |
| 291) | JEV_strain_Ling(L78128)_16mers_2MM.txt | JEV strain Ling(L78128) |
| 292) | JEV_strain_Ling(L78128)_17mers_0MM.txt | JEV strain Ling(L78128) |
| 293) | JEV_strain_Ling(L78128)_17mers_1MM.txt | JEV strain Ling(L78128) |
| 294) | JEV_strain_Ling(L78128)_17mers_2MM.txt | JEV strain Ling(L78128) |
| 295) | JEV_strain_Ling(L78128)_18mers_0MM.txt | JEV strain Ling(L78128) |
| 296) | JEV_strain_Ling(L78128)_18mers_1MM.txt | JEV strain Ling(L78128) |
| 297) | JEV_strain_Ling(L78128)_18mers_2MM.txt | JEV strain Ling(L78128) |
| 298) | JEV_strain_p3(U47032)_16mers_0MM.txt | JEV strain p3(U47032) |
| 299) | JEV_strain_p3(U47032)_16mers_1MM.txt | JEV strain p3(U47032) |
| 300) | JEV_strain_p3(U47032)_16mers_2MM.txt | JEV strain p3(U47032) |
| 301) | JEV_strain_p3(U47032)_17mers_0MM.txt | JEV strain p3(U47032) |
| 302) | JEV_strain_p3(U47032)_17mers_1MM.txt | JEV strain p3(U47032) |
| 303) | JEV_strain_p3(U47032)_17mers_2MM.txt | JEV strain p3(U47032) |
| 304) | JEV_strain_p3(U47032)_18mers_0MM.txt | JEV strain p3(U47032) |
| 305) | JEV_strain_p3(U47032)_18mers_1MM.txt | JEV strain p3(U47032) |
| 306) | JEV_strain_p3(U47032)_18mers_2MM.txt | JEV strain p3(U47032) |
| 307) | JEV_strain_SA(A)_genomic_RNA(D90195)_16mers_0MM.txt | JEV strain SA(A) genomic |
| | | RNA(D90195) |
| 308) | JEV_strain_SA(A)_genomic_RNA(D90195)_16mers_1MM.txt | JEV strain SA(A) genomic |
| | | RNA(D90195) |
| 309) | JEV_strain_SA(A)_genomic_RNA(D90195)_16mers_2MM.txt | JEV strain SA(A) genomic |
| | | RNA(D90195) |
| 310) | JEV_strain_SA(A)_genomic_RNA(D90195)_17mers_0MM.txt | JEV strain SA(A) genomic |
| | | RNA(D90195) |
| 311) | JEV_strain_SA(A)_genomic_RNA(D90195)_17mers_1MM.txt | JEV strain SA(A) genomic |
| | | RNA(D90195) |
| 312) | JEV_strain_SA(A)_genomic_RNA(D90195)_17mers_2MM.txt | JEV strain SA(A) genomic |
| | | RNA(D90195) |
| 313) | JEV_strain_SA(A)_genomic_RNA(D90195)_18mers_0MM.txt | JEV strain SA(A) genomic |
| | | RNA(D90195) |
| 314) | JEV_strain_SA(A)_genomic_RNA(D90195)_18mers_1MM.txt | JEV strain SA(A) genomic |
| | | RNA(D90195) |
| 315) | JEV_strain_SA(A)_genomic_RNA(D90195)_18mers_2MM.txt | JEV strain SA(A) genomic |
| | | RNA(D90195) |
| 316) | JEV_strain_SA(S)_genomic_RNA(D90194)_16mers_0MM.txt | JEV strain SA(S) genomic |
| | | RNA(D90194) |
| 317) | JEV_strain_SA(S)_genomic_RNA(D90194)_16mers_1MM.txt | JEV strain SA(S) genomic |
| | | RNA(D90194) |
| 318) | JEV_strain_SA(S)_genomic_RNA(D90194)_16mers_2MM.txt | JEV strain SA(S) genomic |
| | | RNA(D90194) |
| 319) | JEV_strain_SA(S)_genomic_RNA(D90194)_17mers_0MM.txt | JEV strain SA(S) genomic |
| | | RNA(D90194) |
| 320) | JEV_strain_SA(S)_genomic_RNA(D90194)_17mers_1MM.txt | JEV strain SA(S) genomic |
| | | RNA(D90194) |
| 321) | JEV_strain_SA(S)_genomic_RNA(D90194)_17mers_2MM.txt | JEV strain SA(S) genomic |
| | | RNA(D90194) |
| 322) | JEV_strain_SA(S)_genomic_RNA(D90194)_18mers_0MM.txt | JEV strain SA(S) genomic |
| | | RNA(D90194) |
| 323) | JEV_strain_SA(S)_genomic_RNA(D90194)_18mers_1MM.txt | JEV strain SA(S) genomic |
| | | RNA(D90194) |
| 324) | JEV_strain_SA(S)_genomic_RNA(D90194)_18mers_2MM.txt | JEV strain SA(S) genomic |
| | | RNA(D90194) |
| 325) | JEV_strain_SA14-14-2(AF315119)_16mers_0MM.txt | JEV strain SA14-14-2(AF315119) |
| 326) | JEV_strain_SA14-14-2(AF315119)_16mers_1MM.txt | JEV strain SA14-14-2(AF315119) |
| 327) | JEV_strain_SA14-14-2(AF315119)_16mers_2MM.txt | JEV strain SA14-14-2(AF315119) |
| 328) | JEV_strain_SA14-14-2(AF315119)_17mers_0MM.txt | JEV strain SA14-14-2(AF315119) |
| 329) | JEV_strain_SA14-14-2(AF315119)_17mers_1MM.txt | JEV strain SA14-14-2(AF315119) |
| 330) | JEV_strain_SA14-14-2(AF315119)_17mers_2MM.txt | JEV strain SA14-14-2(AF315119) |
| 331) | JEV_strain_SA14-14-2(AF315119)_18mers_0MM.txt | JEV strain SA14-14-2(AF315119) |
| 332) | JEV_strain_SA14-14-2(AF315119)_18mers_1MM.txt | JEV strain SA14-14-2(AF315119) |
| 333) | JEV_strain_SA14-14-2(AF315119)_18mers_2MM.txt | JEV strain SA14-14-2(AF315119) |
| 334) | JEV_strain_T1P1(AF254453)_16mers_0MM.txt | JEV strain T1P1(AF254453) |
| 335) | JEV_strain_T1P1(AF254453)_16mers_1MM.txt | JEV strain T1P1(AF254453) |
| 336) | JEV_strain_T1P1(AF254453)_16mers_2MM.txt | JEV strain T1P1(AF254453) |
| 337) | JEV_strain_T1P1(AF254453)_17mers_0MM.txt | JEV strain T1P1(AF254453) |
| 338) | JEV_strain_T1P1(AF254453)_17mers_1MM.txt | JEV strain T1P1(AF254453) |
| 339) | JEV_strain_T1P1(AF254453)_17mers_2MM.txt | JEV strain T1P1(AF254453) |
| 340) | JEV_strain_T1P1(AF254453)_18mers_0MM.txt | JEV strain T1P1(AF254453) |
| 341) | JEV_strain_T1P1(AF254453)_18mers_1MM.txt | JEV strain T1P1(AF254453) |
| 342) | JEV_strain_T1P1(AF254453)_18mers_2MM.txt | JEV strain T1P1(AF254453) |
| 343) | JEV_strain_T1P1-L4(AY303792)_16mers_0MM.txt | JEV strain T1P1-L4(AY303792) |
| 344) | JEV_strain_T1P1-L4(AY303792)_16mers_1MM.txt | JEV strain T1P1-L4(AY303792) |
| 345) | JEV_strain_T1P1-L4(AY303792)_16mers_2MM.txt | JEV strain T1P1-L4(AY303792) |
| 346) | JEV_strain_T1P1-L4(AY303792)_17mers_0MM.txt | JEV strain T1P1-L4(AY303792) |
| 347) | JEV_strain_T1P1-L4(AY303792)_17mers_1MM.txt | JEV strain T1P1-L4(AY303792) |
| 348) | JEV_strain_T1P1-L4(AY303792)_17mers_2MM.txt | JEV strain T1P1-L4(AY303792) |
| 349) | JEV_strain_T1P1-L4(AY303792)_18mers_0MM.txt | JEV strain T1P1-L4(AY303792) |
| 350) | JEV_strain_T1P1-L4(AY303792)_18mers_1MM.txt | JEV strain T1P1-L4(AY303792) |
| 351) | JEV_strain_T1P1-L4(AY303792)_18mers_2MM.txt | JEV strain T1P1-L4(AY303792) |
| 352) | JEV_strain_T1P1-S1(AY303791)_16mers_0MM.txt | JEV strain T1P1-S1(AY303791) |
| 353) | JEV_strain_T1P1-S1(AY303791)_16mers_1MM.txt | JEV strain T1P1-S1(AY303791) |
| 354) | JEV_strain_T1P1-S1(AY303791)_16mers_2MM.txt | JEV strain T1P1-S1(AY303791) |
| 355) | JEV_strain_T1P1-S1(AY303791)_17mers_0MM.txt | JEV strain T1P1-S1(AY303791) |
| 356) | JEV_strain_T1P1-S1(AY303791)_17mers_1MM.txt | JEV strain T1P1-S1(AY303791) |
| 357) | JEV_strain_T1P1-S1(AY303791)_17mers_2MM.txt | JEV strain T1P1-S1(AY303791) |
| 358) | JEV_strain_T1P1-S1(AY303791)_18mers_0MM.txt | JEV strain T1P1-S1(AY303791) |
| 359) | JEV_strain_T1P1-S1(AY303791)_18mers_1MM.txt | JEV strain T1P1-S1(AY303791) |
| 360) | JEV_strain_T1P1-S1(AY303791)_18mers_2MM.txt | JEV strain T1P1-S1(AY303791) |
| 361) | JEV_strain_TC(AF098736)_16mers_0MM.txt | JEV strain TC(AF098736) |
| 362) | JEV_strain_TC(AF098736)_16mers_1MM.txt | JEV strain TC(AF098736) |
| 363) | JEV_strain_TC(AF098736)_16mers_2MM.txt | JEV strain TC(AF098736) |
| 364) | JEV_strain_TC(AF098736)_17mers_0MM.txt | JEV strain TC(AF098736) |
| 365) | JEV_strain_TC(AF098736)_17mers_1MM.txt | JEV strain TC(AF098736) |
| 366) | JEV_strain_TC(AF098736)_17mers_2MM.txt | JEV strain TC(AF098736) |
| 367) | JEV_strain_TC(AF098736)_18mers_0MM.txt | JEV strain TC(AF098736) |
| 368) | JEV_strain_TC(AF098736)_18mers_1MM.txt | JEV strain TC(AF098736) |
| 369) | JEV_strain_TC(AF098736)_18mers_2MM.txt | JEV strain TC(AF098736) |
| 370) | JEV_strain_TL(AF098737)_16mers_0MM.txt | JEV strain TL(AF098737) |
| 371) | JEV_strain_TL(AF098737)_16mers_1MM.txt | JEV strain TL(AF098737) |
| 372) | JEV_strain_TL(AF098737)_16mers_2MM.txt | JEV strain TL(AF098737) |
| 373) | JEV_strain_TL(AF098737)_17mers_0MM.txt | JEV strain TL(AF098737) |
| 374) | JEV_strain_TL(AF098737)_17mers_1MM.txt | JEV strain TL(AF098737) |
| 375) | JEV_strain_TL(AF098737)_17mers_2MM.txt | JEV strain TL(AF098737) |
| 376) | JEV_strain_TL(AF098737)_18mers_0MM.txt | JEV strain TL(AF098737) |
| 377) | JEV_strain_TL(AF098737)_18mers_1MM.txt | JEV strain TL(AF098737) |
| 378) | JEV_strain_TL(AF098737)_18mers_2MM.txt | JEV strain TL(AF098737) |
| 379) | JEV_strain_YL_polyprotein_mRNA(AF486638)_16mers_0MM.txt | JEV strain YL polyprotein |
| | | mRNA(AF486638) |
| 380) | JEV_strain_YL_polyprotein_mRNA(AF486638)_16mers_1MM.txt | JEV strain YL polyprotein |
| | | mRNA(AF486638) |
| 381) | JEV_strain_YL_polyprotein_mRNA(AF486638)_16mers_2MM.txt | JEV strain YL polyprotein |
| | | mRNA(AF486638) |
| 382) | JEV_strain_YL_polyprotein_mRNA(AF486638)_17mers_0MM.txt | JEV strain YL polyprotein |
| | | mRNA(AF486638) |
| 383) | JEV_strain_YL_polyprotein_mRNA(AF486638)_17mers_1MM.txt | JEV strain YL polyprotein |
| | | mRNA(AF486638) |
| 384) | JEV_strain_YL_polyprotein_mRNA(AF486638)_17mers_2MM.txt | JEV strain YL polyprotein |
| | | mRNA(AF486638) |
| 385) | JEV_strain_YL_polyprotein_mRNA(AF486638)_18mers_0MM.txt | JEV strain YL polyprotein |
| | | mRNA(AF486638) |
| 386) | JEV_strain_YL_polyprotein_mRNA(AF486638)_18mers_1MM.txt | JEV strain YL polyprotein |
| | | mRNA(AF486638) |
| 387) | JEV_strain_YL_polyprotein_mRNA(AF486638)_18mers_2MM.txt | JEV strain YL polyprotein |
| | | mRNA(AF486638) |
| 388) | Kamiti_River(NC_005064)_16mers_0MM.txt | Kamiti River (NC_005064) |
| 389) | Kamiti_River(NC_005064)_16mers_1MM.txt | Kamiti River (NC_005064) |
| 390) | Kamiti_River(NC_005064)_16mers_2MM.txt | Kamiti River (NC_005064) |
| 391) | Kamiti_River(NC_005064)_17mers_0MM.txt | Kamiti River (NC_005064) |
| 392) | Kamiti_River(NC_005064)_17mers_1MM.txt | Kamiti River (NC_005064) |
| 393) | Kamiti_River(NC_005064)_17mers_2MM.txt | Kamiti River (NC_005064) |
| 394) | Kamiti_River(NC_005064)_18mers_0MM.txt | Kamiti River (NC_005064) |
| 395) | Kamiti_River(NC_005064)_18mers_1MM.txt | Kamiti River (NC_005064) |
| 396) | Kamiti_River(NC_005064)_18mers_2MM.txt | Kamiti River (NC_005064) |
| 397) | KRV_isolate_SR-75_polyprotein_precursor(AY149904)_16mers_0MM.txt | KRV isolate SR-75 |
| | | polyprotein precursor(AY149904) |
| 398) | KRV_isolate_SR-75_polyprotein_precursor(AY149904)_16mers_1MM.txt | KRV isolate SR-75 |
| | | polyprotein precursor(AY149904) |
| 399) | KRV_isolate_SR-75_polyprotein_precursor(AY149904)_16mers_2MM.txt | KRV isolate SR-75 |
| | | polyprotein precursor(AY149904) |
| 400) | KRV_isolate_SR-75_polyprotein_precursor(AY149904)_17mers_0MM.txt | KRV isolate SR-75 |
| | | polyprotein precursor(AY149904) |
| 401) | KRV_isolate_SR-75_polyprotein_precursor(AY149904)_17mers_1MM.txt | KRV isolate SR-75 |
| | | polyprotein precursor(AY149904) |
| 402) | KRV_isolate_SR-75_polyprotein_precursor(AY149904)_17mers_2MM.txt | KRV isolate SR-75 |
| | | polyprotein precursor(AY149904) |
| 403) | KRV_isolate_SR-75_polyprotein_precursor(AY149904)_18mers_0MM.txt | KRV isolate SR-75 |
| | | polyprotein precursor(AY149904) |
| 404) | KRV_isolate_SR-75_polyprotein_precursor(AY149904)_18mers_1MM.txt | KRV isolate SR-75 |
| | | polyprotein precursor(AY149904) |
| 405) | KRV_isolate_SR-75_polyprotein_precursor(AY149904)_18mers_2MM.txt | KRV isolate SR-75 |
| | | polyprotein precursor(AY149904) |
| 406) | Kunjin_virus_clone_FLSDX_polyprotein_mRNA(AY274504)_16mers_0MM.txt | Kunjin virus |
| | | clone FLSDX polyprotein mRNA(AY274504) |
| 407) | Kunjin_virus_clone_FLSDX_polyprotein_mRNA(AY274504)_16mers_1MM.txt | Kunjin virus |
| | | clone FLSDX polyprotein mRNA(AY274504) |
| 408) | Kunjin_virus_clone_FLSDX_polyprotein_mRNA(AY274504)_16mers_2MM.txt | Kunjin virus |
| | | clone FLSDX polyprotein mRNA(AY274504) |
| 409) | Kunjin_virus_clone_FLSDX_polyprotein_mRNA(AY274504)_17mers_0MM.txt | Kunjin virus |
| | | clone FLSDX polyprotein mRNA(AY274504) |
| 410) | Kunjin_virus_clone_FLSDX_polyprotein_mRNA(AY274504)_17mers_1MM.txt | Kunjin virus |
| | | clone FLSDX polyprotein mRNA(AY274504) |
| 411) | Kunjin_virus_clone_FLSDX_polyprotein_mRNA(AY274504)_17mers_2MM.txt | Kunjin virus |
| | | clone FLSDX polyprotein mRNA(AY274504) |
| 412) | Kunjin_virus_clone_FLSDX_polyprotein_mRNA(AY274504)_18mers_0MM.txt | Kunjin virus |
| | | clone FLSDX polyprotein mRNA(AY274504) |
| 413) | Kunjin_virus_clone_FLSDX_polyprotein_mRNA(AY274504)_18mers_1MM.txt | Kunjin virus |
| | | clone FLSDX polyprotein mRNA(AY274504) |
| 414) | Kunjin_virus_clone_FLSDX_polyprotein_mRNA(AY274504)_18mers_2MM.txt | Kunjin virus |
| | | clone FLSDX polyprotein mRNA(AY274504) |
| 415) | Kunjin_virus_clone_pAKUN_polyprotein_mRNA(AY274505)_16mers_0MM.txt | Kunjin virus |
| | | clone pAKUN polyprotein mRNA(AY274505) |
| 416) | Kunjin_virus_clone_pAKUN_polyprotein_mRNA(AY274505)_16mers_1MM.txt | Kunjin virus |
| | | clone pAKUN polyprotein mRNA(AY274505) |
| 417) | Kunjin_virus_clone_pAKUN_polyprotein_mRNA(AY274505)_16mers_2MM.txt | Kunjin virus |
| | | clone pAKUN polyprotein mRNA(AY274505) |
| 418) | Kunjin_virus_clone_pAKUN_polyprotein_mRNA(AY274505)_17mers_0MM.txt | Kunjin virus |
| | | clone pAKUN polyprotein mRNA(AY274505) |
| 419) | Kunjin_virus_clone_pAKUN_polyprotein_mRNA(AY274505)_17mers_1MM.txt | Kunjin virus |
| | | clone pAKUN polyprotein mRNA(AY274505) |
| 420) | Kunjin_virus_clone_pAKUN_polyprotein_mRNA(AY274505)_17mers_2MM.txt | Kunjin virus |
| | | clone pAKUN polyprotein mRNA(AY274505) |
| 421) | Kunjin_virus_clone_pAKUN_polyprotein_mRNA(AY274505)_18mers_0MM.txt | Kunjin virus |
| | | clone pAKUN polyprotein mRNA(AY274505) |
| 422) | Kunjin_virus_clone_pAKUN_polyprotein_mRNA(AY274505)_18mers_1MM.txt | Kunjin virus |
| | | clone pAKUN polyprotein mRNA(AY274505) |
| 423) | Kunjin_virus_clone_pAKUN_polyprotein_mRNA(AY274505)_18mers_2MM.txt | Kunjin virus |
| | | clone pAKUN polyprotein mRNA(AY274505) |
| 424) | Kunjin_virus_gene_for_polyprotein(D00246)_16mers_0MM.txt | Kunjin virus gene for |
| | | polyprotein(D00246) |
| 425) | Kunjin_virus_gene_for_polyprotein(D00246)_16mers_1MM.txt | Kunjin virus gene for |
| | | polyprotein(D00246) |
| 426) | Kunjin_virus_gene_for_polyprotein(D00246)_16mers_2MM.txt | Kunjin virus gene for |
| | | polyprotein(D00246) |
| 427) | Kunjin_virus_gene_for_polyprotein(D00246)_17mers_0MM.txt | Kunjin virus gene for |
| | | polyprotein(D00246) |
| 428) | Kunjin_virus_gene_for_polyprotein(D00246)_17mers_1MM.txt | Kunjin virus gene for |
| | | polyprotein(D00246) |
| 429) | Kunjin_virus_gene_for_polyprotein(D00246)_17mers_2MM.txt | Kunjin virus gene for |
| | | polyprotein(D00246) |
| 430) | Kunjin_virus_gene_for_polyprotein(D00246)_18mers_0MM.txt | Kunjin virus gene for |
| | | polyprotein(D00246) |
| 431) | Kunjin_virus_gene_for_polyprotein(D00246)_18mers_1MM.txt | Kunjin virus gene for |
| | | polyprotein(D00246) |
| 432) | Kunjin_virus_gene_for_polyprotein(D00246)_18mers_2MM.txt | Kunjin virus gene for |
| | | polyprotein(D00246) |
| 433) | Langat(NC_003690)_16mers_0MM.txt | Langat (NC_003690) |
| 434) | Langat(NC_003690)_16mers_1MM.txt | Langat (NC_003690) |
| 435) | Langat(NC_003690)_16mers_2MM.txt | Langat (NC_003690) |
| 436) | Langat(NC_003690)_17mers_0MM.txt | Langat (NC_003690) |
| 437) | Langat(NC_003690)_17mers_1MM.txt | Langat (NC_003690) |
| 438) | Langat(NC_003690)_17mers_2MM.txt | Langat (NC_003690) |
| 439) | Langat(NC_003690)_18mers_0MM.txt | Langat (NC_003690) |
| 440) | Langat(NC_003690)_18mers_1MM.txt | Langat (NC_003690) |
| 441) | Langat(NC_003690)_18mers_2MM.txt | Langat (NC_003690) |
| 442) | Louping_ill(NC_001809)_16mers_0MM.txt | Louping ill (NC_001809) |
| 443) | Louping_ill(NC_001809)_16mers_1MM.txt | Louping ill (NC_001809) |
| 444) | Louping_ill(NC_001809)_16mers_2MM.txt | Louping ill (NC_001809) |
| 445) | Louping_ill(NC_001809)_17mers_0MM.txt | Louping ill (NC_001809) |
| 446) | Louping_ill(NC_001809)_17mers_1MM.txt | Louping ill (NC_001809) |
| 447) | Louping_ill(NC_001809)_17mers_2MM.txt | Louping ill (NC_001809) |
| 448) | Louping_ill(NC_001809)_18mers_0MM.txt | Louping ill (NC_001809) |
| 449) | Louping_ill(NC_001809)_18mers_1MM.txt | Louping ill (NC_001809) |
| 450) | Louping_ill(NC_001809)_18mers_2MM.txt | Louping ill (NC_001809) |
| 451) | LV_attenuated_strain_E5_polyprotein_gene(AF253420)_16mers_0MM.txt | LV attenuated strain E5 |
| | | polyprotein gene(AF253420) |
| 452) | LV_attenuated_strain_E5_polyprotein_gene(AF253420)_16mers_1MM.txt | LV attenuated strain E5 |
| | | polyprotein gene(AF253420) |
| 453) | LV_attenuated_strain_E5_polyprotein_gene(AF253420)_16mers_2MM.txt | LV attenuated strain E5 |
| | | polyprotein gene(AF253420) |
| 454) | LV_attenuated_strain_E5_polyprotein_gene(AF253420)_17mers_0MM.txt | LV attenuated strain E5 |
| | | polyprotein gene(AF253420) |
| 455) | LV_attenuated_strain_E5_polyprotein_gene(AF253420)_17mers_1MM.txt | LV attenuated strain E5 |
| | | polyprotein gene(AF253420) |
| 456) | LV_attenuated_strain_E5_polyprotein_gene(AF253420)_17mers_2MM.txt | LV attenuated strain E5 |
| | | polyprotein gene(AF253420) |
| 457) | LV_attenuated_strain_E5_polyprotein_gene(AF253420)_18mers_0MM.txt | LV attenuated strain E5 |
| | | polyprotein gene(AF253420) |
| 458) | LV_attenuated_strain_E5_polyprotein_gene(AF253420)_18mers_1MM.txt | LV attenuated strain E5 |
| | | polyprotein gene(AF253420) |
| 459) | LV_attenuated_strain_E5_polyprotein_gene(AF253420)_18mers_2MM.txt | LV attenuated strain E5 |
| | | polyprotein gene(AF253420) |
| 460) | Modoc(NC_003635)_16mers_0MM.txt | Modoc (NC_003635) |
| 461) | Modoc(NC_003635)_16mers_1MM.txt | Modoc (NC_003635) |
| 462) | Modoc(NC_003635)_16mers_2MM.txt | Modoc (NC_003635) |
| 463) | Modoc(NC_003635)_17mers_0MM.txt | Modoc (NC_003635) |
| 464) | Modoc(NC_003635)_17mers_1MM.txt | Modoc (NC_003635) |
| 465) | Modoc(NC_003635)_17mers_2MM.txt | Modoc (NC_003635) |
| 466) | Modoc(NC_003635)_18mers_0MM.txt | Modoc (NC_003635) |
| 467) | Modoc(NC_003635)_18mers_1MM.txt | Modoc (NC_003635) |
| 468) | Modoc(NC_003635)_18mers_2MM.txt | Modoc (NC_003635) |
| 469) | Montana_myotis_leukoencephalitis(NC_004119)_16mers_0MM.txt | Montana myotis |
| | | leukoencephalitis (NC_004119) |
| 470) | Montana_myotis_leukoencephalitis(NC_004119)_16mers_1MM.txt | Montana myotis |
| | | leukoencephalitis (NC_004119) |
| 471) | Montana_myotis_leukoencephalitis(NC_004119)_16mers_2MM.txt | Montana myotis |
| | | leukoencephalitis (NC_004119) |
| 472) | Montana_myotis_leukoencephalitis(NC_004119)_17mers_0MM.txt | Montana myotis |
| | | leukoencephalitis (NC_004119) |
| 473) | Montana_myotis_leukoencephalitis(NC_004119)_17mers_1MM.txt | Montana myotis |
| | | leukoencephalitis (NC_004119) |
| 474) | Montana_myotis_leukoencephalitis(NC_004119)_17mers_2MM.txt | Montana myotis |
| | | leukoencephalitis (NC_004119) |
| 475) | Montana_myotis_leukoencephalitis(NC_004119)_18mers_0MM.txt | Montana myotis |
| | | leukoencephalitis (NC_004119) |
| 476) | Montana_myotis_leukoencephalitis(NC_004119)_18mers_1MM.txt | Montana myotis |
| | | leukoencephalitis (NC_004119) |
| 477) | Montana_myotis_leukoencephalitis(NC_004119)_18mers_2MM.txt | Montana myotis |
| | | leukoencephalitis (NC_004119) |
| 478) | Murray_Valley_encephalitis(NC_000943)_16mers_0MM.txt | Murray Valley encephalitis |
| | | (NC_000943) |
| 479) | Murray_Valley_encephalitis(NC_000943)_16mers_1MM.txt | Murray Valley encephalitis |
| | | (NC_000943) |
| 480) | Murray_Valley_encephalitis(NC_000943)_16mers_2MM.txt | Murray Valley encephalitis |
| | | (NC_000943) |
| 481) | Murray_Valley_encephalitis(NC_000943)_17mers_0MM.txt | Murray Valley encephalitis |
| | | (NC_000943) |
| 482) | Murray_Valley_encephalitis(NC_000943)_17mers_1MM.txt | Murray Valley encephalitis |
| | | (NC_000943) |
| 483) | Murray_Valley_encephalitis(NC_000943)_17mers_2MM.txt | Murray Valley encephalitis |
| | | (NC_000943) |
| 484) | Murray_Valley_encephalitis(NC_000943)_18mers_0MM.txt | Murray Valley encephalitis |
| | | (NC_000943) |
| 485) | Murray_Valley_encephalitis(NC_000943)_18mers_1MM.txt | Murray Valley encephalitis |
| | | (NC_000943) |
| 486) | Murray_Valley_encephalitis(NC_000943)_18mers_2MM.txt | Murray Valley encephalitis |
| | | (NC_000943) |
| 487) | OHFV_strain_Kubrin_16mers_0MM.txt | OHFV strain Kubrin |
| 488) | OHFV_strain_Kubrin_16mers_1MM.txt | OHFV strain Kubrin |
| 489) | OHFV_strain_Kubrin_16mers_2MM.txt | OHFV strain Kubrin |
| 490) | OHFV_strain_Kubrin_17mers_0MM.txt | OHFV strain Kubrin |
| 491) | OHFV_strain_Kubrin_17mers_1MM.txt | OHFV strain Kubrin |
| 492) | OHFV_strain_Kubrin_17mers_2MM.txt | OHFV strain Kubrin |
| 493) | OHFV_strain_Kubrin_18mers_0MM.txt | OHFV strain Kubrin |
| 494) | OHFV_strain_Kubrin_18mers_1MM.txt | OHFV strain Kubrin |
| 495) | OHFV_strain_Kubrin_18mers_2MM.txt | OHFV strain Kubrin |
| 496) | Omsk_hemorrhagic_fever(NC_005062)_16mers_0MM.txt | Omsk hemorrhagic fever |
| | | (NC_005062) |
| 497) | Omsk_hemorrhagic_fever(NC_005062)_16mers_1MM.txt | Omsk hemorrhagic fever |
| | | (NC_005062) |
| 498) | Omsk_hemorrhagic_fever(NC_005062)_16mers_2MM.txt | Omsk hemorrhagic fever |
| | | (NC_005062) |
| 499) | Omsk_hemorrhagic_fever(NC_005062)_17mers_0MM.txt | Omsk hemorrhagic fever |
| | | (NC_005062) |
| 500) | Omsk_hemorrhagic_fever(NC_005062)_17mers_1MM.txt | Omsk hemorrhagic fever |
| | | (NC_005062) |
| 501) | Omsk_hemorrhagic_fever(NC_005062)_17mers_2MM.txt | Omsk hemorrhagic fever |
| | | (NC_005062) |
| 502) | Omsk_hemorrhagic_fever(NC_005062)_18mers_0MM.txt | Omsk hemorrhagic fever |
| | | (NC_005062) |
| 503) | Omsk_hemorrhagic_fever(NC_005062)_18mers_1MM.txt | Omsk hemorrhagic fever |
| | | (NC_005062) |
| 504) | Omsk_hemorrhagic_fever(NC_005062)_18mers_2MM.txt | Omsk hemorrhagic fever |
| | | (NC_005062) |
| 505) | Powassan(NC_003687)_16mers_0MM.txt | Powassan (NC_003687) |
| 506) | Powassan(NC_003687)_16mers_1MM.txt | Powassan (NC_003687) |
| 507) | Powassan(NC_003687)_16mers_2MM.txt | Powassan (NC_003687) |
| 508) | Powassan(NC_003687)_17mers_0MM.txt | Powassan (NC_003687) |
| 509) | Powassan(NC_003687)_17mers_1MM.txt | Powassan (NC_003687) |
| 510) | Powassan(NC_003687)_17mers_2MM.txt | Powassan (NC_003687) |
| 511) | Powassan(NC_003687)_18mers_0MM.txt | Powassan (NC_003687) |
| 512) | Powassan(NC_003687)_18mers_1MM.txt | Powassan (NC_003687) |
| 513) | Powassan(NC_003687)_18mers_2MM.txt | Powassan (NC_003687) |
| 514) | Rio_Bravo(NC_003675)_16mers_0MM.txt | Rio Bravo (NC_003675) |
| 515) | Rio_Bravo(NC_003675)_16mers_1MM.txt | Rio Bravo (NC_003675) |
| 516) | Rio_Bravo(NC_003675)_16mers_2MM.txt | Rio Bravo (NC_003675) |
| 517) | Rio_Bravo(NC_003675)_17mers_0MM.txt | Rio Bravo (NC_003675) |
| 518) | Rio_Bravo(NC_003675)_17mers_1MM.txt | Rio Bravo (NC_003675) |
| 519) | Rio_Bravo(NC_003675)_17mers_2MM.txt | Rio Bravo (NC_003675) |
| 520) | Rio_Bravo(NC_003675)_18mers_0MM.txt | Rio Bravo (NC_003675) |
| 521) | Rio_Bravo(NC_003675)_18mers_1MM.txt | Rio Bravo (NC_003675) |
| 522) | Rio_Bravo(NC_003675)_18mers_2MM.txt | Rio Bravo (NC_003675) |
| 523) | Tamana_bat(NC_003996)_16mers_0MM.txt | Tamana bat (NC_003996) |
| 524) | Tamana_bat(NC_003996)_16mers_1MM.txt | Tamana bat (NC_003996) |
| 525) | Tamana_bat(NC_003996)_16mers_2MM.txt | Tamana bat (NC_003996) |
| 526) | Tamana_bat(NC_003996)_17mers_0MM.txt | Tamana bat (NC_003996) |
| 527) | Tamana_bat(NC_003996)_17mers_1MM.txt | Tamana bat (NC_003996) |
| 528) | Tamana_bat(NC_003996)_17mers_2MM.txt | Tamana bat (NC_003996) |
| 529) | Tamana_bat(NC_003996)_18mers_0MM.txt | Tamana bat (NC_003996) |
| 530) | Tamana_bat(NC_003996)_18mers_1MM.txt | Tamana bat (NC_003996) |
| 531) | Tamana_bat(NC_003996)_18mers_2MM.txt | Tamana bat (NC_003996) |
| 532) | TBEV_263_polyprotein(U27491)_16mers_0MM.txt | TBEV 263 polyprotein(U27491) |
| 533) | TBEV_263_polyprotein(U27491)_16mers_1MM.txt | TBEV 263 polyprotein(U27491) |
| 534) | TBEV_263_polyprotein(U27491)_16mers_2MM.txt | TBEV 263 polyprotein(U27491) |
| 535) | TBEV_263_polyprotein(U27491)_17mers_0MM.txt | TBEV 263 polyprotein(U27491) |
| 536) | TBEV_263_polyprotein(U27491)_17mers_1MM.txt | TBEV 263 polyprotein(U27491) |
| 537) | TBEV_263_polyprotein(U27491)_17mers_2MM.txt | TBEV 263 polyprotein(U27491) |
| 538) | TBEV_263_polyprotein(U27491)_18mers_0MM.txt | TBEV 263 polyprotein(U27491) |
| 539) | TBEV_263_polyprotein(U27491)_18mers_1MM.txt | TBEV 263 polyprotein(U27491) |
| 540) | TBEV_263_polyprotein(U27491)_18mers_2MM.txt | TBEV 263 polyprotein(U27491) |
| 541) | TBEV_Hypr_polyprotein_gene(U39292)_16mers_0MM.txt | TBEV Hypr polyprotein |
| | | gene(U39292) |
| 542) | TBEV_Hypr_polyprotein_gene(U39292)_16mers_1MM.txt | TBEV Hypr polyprotein |
| | | gene(U39292) |
| 543) | TBEV_Hypr_polyprotein_gene(U39292)_16mers_2MM.txt | TBEV Hypr polyprotein |
| | | gene(U39292) |
| 544) | TBEV_Hypr_polyprotein_gene(U39292)_17mers_0MM.txt | TBEV Hypr polyprotein |
| | | gene(U39292) |
| 545) | TBEV_Hypr_polyprotein_gene(U39292)_17mers_1MM.txt | TBEV Hypr polyprotein |
| | | gene(U39292) |
| 546) | TBEV_Hypr_polyprotein_gene(U39292)_17mers_2MM.txt | TBEV Hypr polyprotein |
| | | gene(U39292) |
| 547) | TBEV_Hypr_polyprotein_gene(U39292)_18mers_0MM.txt | TBEV Hypr polyprotein |
| | | gene(U39292) |
| 548) | TBEV_Hypr_polyprotein_gene(U39292)_18mers_1MM.txt | TBEV Hypr polyprotein |
| | | gene(U39292) |
| 549) | TBEV_Hypr_polyprotein_gene(U39292)_18mers_2MM.txt | TBEV Hypr polyprotein |
| | | gene(U39292) |
| 550) | TBEV_polyprotein_gene(L40361)_16mers_0MM.txt | TBEV polyprotein gene(L40361) |
| 551) | TBEV_polyprotein_gene(L40361)_16mers_1MM.txt | TBEV polyprotein gene(L40361) |
| 552) | TBEV_polyprotein_gene(L40361)_16mers_2MM.txt | TBEV polyprotein gene(L40361) |
| 553) | TBEV_polyprotein_gene(L40361)_17mers_0MM.txt | TBEV polyprotein gene(L40361) |
| 554) | TBEV_polyprotein_gene(L40361)_17mers_1MM.txt | TBEV polyprotein gene(L40361) |
| 555) | TBEV_polyprotein_gene(L40361)_17mers_2MM.txt | TBEV polyprotein gene(L40361) |
| 556) | TBEV_polyprotein_gene(L40361)_18mers_0MM.txt | TBEV polyprotein gene(L40361) |
| 557) | TBEV_polyprotein_gene(L40361)_18mers_1MM.txt | TBEV polyprotein gene(L40361) |
| 558) | TBEV_polyprotein_gene(L40361)_18mers_2MM.txt | TBEV polyprotein gene(L40361) |
| 559) | TBEV_strain_Oshima(AB062063)_16mers_0MM.txt | TBEV strain Oshima(AB062063) |
| 560) | TBEV_strain_Oshima(AB062063)_16mers_1MM.txt | TBEV strain Oshima(AB062063) |
| 561) | TBEV_strain_Oshima(AB062063)_16mers_2MM.txt | TBEV strain Oshima(AB062063) |
| 562) | TBEV_strain_Oshima(AB062063)_17mers_0MM.txt | TBEV strain Oshima(AB062063) |
| 563) | TBEV_strain_Oshima(AB062063)_17mers_1MM.txt | TBEV strain Oshima(AB062063) |
| 564) | TBEV_strain_Oshima(AB062063)_17mers_2MM.txt | TBEV strain Oshima(AB062063) |
| 565) | TBEV_strain_Oshima(AB062063)_18mers_0MM.txt | TBEV strain Oshima(AB062063) |
| 566) | TBEV_strain_Oshima(AB062063)_18mers_1MM.txt | TBEV strain Oshima(AB062063) |
| 567) | TBEV_strain_Oshima(AB062063)_18mers_2MM.txt | TBEV strain Oshima(AB062063) |
| 568) | TBEV_strain_Senzhang(AY182009)_16mers_0MM.txt | TBEV strain Senzhang(AY182009) |
| 569) | TBEV_strain_Senzhang(AY182009)_16mers_1MM.txt | TBEV strain Senzhang(AY182009) |
| 570) | TBEV_strain_Senzhang(AY182009)_16mers_2MM.txt | TBEV strain Senzhang(AY182009) |
| 571) | TBEV_strain_Senzhang(AY182009)_17mers_0MM.txt | TBEV strain Senzhang(AY182009) |
| 572) | TBEV_strain_Senzhang(AY182009)_17mers_1MM.txt | TBEV strain Senzhang(AY182009) |
| 573) | TBEV_strain_Senzhang(AY182009)_17mers_2MM.txt | TBEV strain Senzhang(AY182009) |
| 574) | TBEV_strain_Senzhang(AY182009)_18mers_0MM.txt | TBEV strain Senzhang(AY182009) |
| 575) | TBEV_strain_Senzhang(AY182009)_18mers_1MM.txt | TBEV strain Senzhang(AY182009) |
| 576) | TBEV_strain_Senzhang(AY182009)_18mers_2MM.txt | TBEV strain Senzhang(AY182009) |
| 577) | TBEV_strain_Sofjin-HO(AB062064)_16mers_0MM.txt | TBEV strain Sofjin-HO(AB062064) |
| 578) | TBEV_strain_Sofjin-HO(AB062064)_16mers_1MM.txt | TBEV strain Sofjin-HO(AB062064) |
| 579) | TBEV_strain_Sofjin-HO(AB062064)_16mers_2MM.txt | TBEV strain Sofjin-HO(AB062064) |
| 580) | TBEV_strain_Sofjin-HO(AB062064)_17mers_0MM.txt | TBEV strain Sofjin-HO(AB062064) |
| 581) | TBEV_strain_Sofjin-HO(AB062064)_17mers_1MM.txt | TBEV strain Sofjin-HO(AB062064) |
| 582) | TBEV_strain_Sofjin-HO(AB062064)_17mers_2MM.txt | TBEV strain Sofjin-HO(AB062064) |
| 583) | TBEV_strain_Sofjin-HO(AB062064)_18mers_0MM.txt | TBEV strain Sofjin-HO(AB062064) |
| 584) | TBEV_strain_Sofjin-HO(AB062064)_18mers_1MM.txt | TBEV strain Sofjin-HO(AB062064) |
| 585) | TBEV_strain_Sofjin-HO(AB062064)_18mers_2MM.txt | TBEV strain Sofjin-HO(AB062064) |
| 586) | TBEV_strain_Vasilchenko(AF069066)_16mers_0MM.txt | TBEV strain |
| | | Vasilchenko(AF069066) |
| 587) | TBEV_strain_Vasilchenko(AF069066)_16mers_1MM.txt | TBEV strain |
| | | Vasilchenko(AF069066) |
| 588) | TBEV_strain_Vasilchenko(AF069066)_16mers_2MM.txt | TBEV strain |
| | | Vasilchenko(AF069066) |
| 589) | TBEV_strain_Vasilchenko(AF069066)_17mers_0MM.txt | TBEV strain |
| | | Vasilchenko(AF069066) |
| 590) | TBEV_strain_Vasilchenko(AF069066)_17mers_1MM.txt | TBEV strain |
| | | Vasilchenko(AF069066) |
| 591) | TBEV_strain_Vasilchenko(AF069066)_17mers_2MM.txt | TBEV strain |
| | | Vasilchenko(AF069066) |
| 592) | TBEV_strain_Vasilchenko(AF069066)_18mers_0MM.txt | TBEV strain |
| | | Vasilchenko(AF069066) |
| 593) | TBEV_strain_Vasilchenko(AF069066)_18mers_1MM.txt | TBEV strain |
| | | Vasilchenko(AF069066) |
| 594) | TBEV_strain_Vasilchenko(AF069066)_18mers_2MM.txt | TBEV strain |
| | | Vasilchenko(AF069066) |
| 595) | TBEV_strain_Zausaev(AF527415)_16mers_0MM.txt | TBEV strain Zausaev(AF527415) |
| 596) | TBEV_strain_Zausaev(AF527415)_16mers_1MM.txt | TBEV strain Zausaev(AF527415) |
| 597) | TBEV_strain_Zausaev(AF527415)_16mers_2MM.txt | TBEV strain Zausaev(AF527415) |
| 598) | TBEV_strain_Zausaev(AF527415)_17mers_0MM.txt | TBEV strain Zausaev(AF527415) |
| 599) | TBEV_strain_Zausaev(AF527415)_17mers_1MM.txt | TBEV strain Zausaev(AF527415) |
| 600) | TBEV_strain_Zausaev(AF527415)_17mers_2MM.txt | TBEV strain Zausaev(AF527415) |
| 601) | TBEV_strain_Zausaev(AF527415)_18mers_0MM.txt | TBEV strain Zausaev(AF527415) |
| 602) | TBEV_strain_Zausaev(AF527415)_18mers_1MM.txt | TBEV strain Zausaev(AF527415) |
| 603) | TBEV_strain_Zausaev(AF527415)_18mers_2MM.txt | TBEV strain Zausaev(AF527415) |
| 604) | Tick-borne_encephalitis(NC_001672)_16mers_0MM.txt | Tick-borne encephalitis (NC_001672) |
| 605) | Tick-borne_encephalitis(NC_001672)_16mers_1MM.txt | Tick-borne encephalitis (NC_001672) |
| 606) | Tick-borne_encephalitis(NC_001672)_16mers_2MM.txt | Tick-borne encephalitis (NC_001672) |
| 607) | Tick-borne_encephalitis(NC_001672)_17mers_0MM.txt | Tick-borne encephalitis (NC_001672) |
| 608) | Tick-borne_encephalitis(NC_001672)_17mers_1MM.txt | Tick-borne encephalitis (NC_001672) |
| 609) | Tick-borne_encephalitis(NC_001672)_17mers_2MM.txt | Tick-borne encephalitis (NC_001672) |
| 610) | Tick-borne_encephalitis(NC_001672)_18mers_0MM.txt | Tick-borne encephalitis (NC_001672) |
| 611) | Tick-borne_encephalitis(NC_001672)_18mers_1MM.txt | Tick-borne encephalitis (NC_001672) |
| 612) | Tick-borne_encephalitis(NC_001672)_18mers_2MM.txt | Tick-borne encephalitis (NC_001672) |
| 613) | Yellow_fever(NC_002031)_16mers_0MM.txt | Yellow fever (NC_002031) |
| 614) | Yellow_fever(NC_002031)_16mers_1MM.txt | Yellow fever (NC_002031) |
| 615) | Yellow_fever(NC_002031)_16mers_2MM.txt | Yellow fever (NC_002031) |
| 616) | Yellow_fever(NC_002031)_17mers_0MM.txt | Yellow fever (NC_002031) |
| 617) | Yellow_fever(NC_002031)_17mers_1MM.txt | Yellow fever (NC_002031) |
| 618) | Yellow_fever(NC_002031)_17mers_2MM.txt | Yellow fever (NC_002031) |
| 619) | Yellow_fever(NC_002031)_18mers_0MM.txt | Yellow fever (NC_002031) |
| 620) | Yellow_fever(NC_002031)_18mers_1MM.txt | Yellow fever (NC_002031) |
| 621) | Yellow_fever(NC_002031)_18mers_2MM.txt | Yellow fever (NC_002031) |
| 622) | YFV_clone_HONG1_polyprotein_gene(AF052437)_16mers_0MM.txt | YFV clone HONG1 |
| | | polyprotein gene(AF052437) |
| 623) | YFV_clone_HONG1_polyprotein_gene(AF052437)_16mers_1MM.txt | YFV clone HONG1 |
| | | polyprotein gene(AF052437) |
| 624) | YFV_clone_HONG1_polyprotein_gene(AF052437)_16mers_2MM.txt | YFV clone HONG1 |
| | | polyprotein gene(AF052437) |
| 625) | YFV_clone_HONG1_polyprotein_gene(AF052437)_17mers_0MM.txt | YFV clone HONG1 |
| | | polyprotein gene(AF052437) |
| 626) | YFV_clone_HONG1_polyprotein_gene(AF052437)_17mers_1MM.txt | YFV clone HONG1 |
| | | polyprotein gene(AF052437) |
| 627) | YFV_clone_HONG1_polyprotein_gene(AF052437)_17mers_2MM.txt | YFV clone HONG1 |
| | | polyprotein gene(AF052437) |
| 628) | YFV_clone_HONG1_polyprotein_gene(AF052437)_18mers_0MM.txt | YFV clone HONG1 |
| | | polyprotein gene(AF052437) |
| 629) | YFV_clone_HONG1_polyprotein_gene(AF052437)_18mers_1MM.txt | YFV clone HONG1 |
| | | polyprotein gene(AF052437) |
| 630) | YFV_clone_HONG1_polyprotein_gene(AF052437)_18mers_2MM.txt | YFV clone HONG1 |
| | | polyprotein gene(AF052437) |
| 631) | YFV_clone_HONG10_polyprotein_gene(AF052446)_16mers_0MM.txt | YFV clone HONG10 |
| | | polyprotein gene(AF052446) |
| 632) | YFV_clone_HONG10_polyprotein_gene(AF052446)_16mers_1MM.txt | YFV clone HONG10 |
| | | polyprotein gene(AF052446) |
| 633) | YFV_clone_HONG10_polyprotein_gene(AF052446)_16mers_2MM.txt | YFV clone HONG10 |
| | | polyprotein gene(AF052446) |
| 634) | YFV_clone_HONG10_polyprotein_gene(AF052446)_17mers_0MM.txt | YFV clone HONG10 |
| | | polyprotein gene(AF052446) |
| 635) | YFV_clone_HONG10_polyprotein_gene(AF052446)_17mers_1MM.txt | YFV clone HONG10 |
| | | polyprotein gene(AF052446) |
| 636) | YFV_clone_HONG10_polyprotein_gene(AF052446)_17mers_2MM.txt | YFV clone HONG10 |
| | | polyprotein gene(AF052446) |
| 637) | YFV_clone_HONG10_polyprotein_gene(AF052446)_18mers_0MM.txt | YFV clone HONG10 |
| | | polyprotein gene(AF052446) |
| 638) | YFV_clone_HONG10_polyprotein_gene(AF052446)_18mers_1MM.txt | YFV clone HONG10 |
| | | polyprotein gene(AF052446) |
| 639) | YFV_clone_HONG10_polyprotein_gene(AF052446)_18mers_2MM.txt | YFV clone HONG10 |
| | | polyprotein gene(AF052446) |
| 640) | YFV_clone_HONG2_polyprotein_gene(AF052438)_16mers_0MM.txt | YFV clone HONG2 |
| | | polyprotein gene(AF052438) |
| 641) | YFV_clone_HONG2_polyprotein_gene(AF052438)_16mers_1MM.txt | YFV clone HONG2 |
| | | polyprotein gene(AF052438) |
| 642) | YFV_clone_HONG2_polyprotein_gene(AF052438)_16mers_2MM.txt | YFV clone HONG2 |
| | | polyprotein gene(AF052438) |
| 643) | YFV_clone_HONG2_polyprotein_gene(AF052438)_17mers_0MM.txt | YFV clone HONG2 |
| | | polyprotein gene(AF052438) |
| 644) | YFV_clone_HONG2_polyprotein_gene(AF052438)_17mers_1MM.txt | YFV clone HONG2 |
| | | polyprotein gene(AF052438) |
| 645) | YFV_clone_HONG2_polyprotein_gene(AF052438)_17mers_2MM.txt | YFV clone HONG2 |
| | | polyprotein gene(AF052438) |
| 646) | YFV_clone_HONG2_polyprotein_gene(AF052438)_18mers_0MM.txt | YFV clone HONG2 |
| | | polyprotein gene(AF052438) |
| 647) | YFV_clone_HONG2_polyprotein_gene(AF052438)_18mers_1MM.txt | YFV clone HONG2 |
| | | polyprotein gene(AF052438) |
| 648) | YFV_clone_HONG2_polyprotein_gene(AF052438)_18mers_2MM.txt | YFV clone HONG2 |
| | | polyprotein gene(AF052438) |
| 649) | YFV_clone_HONG3_polyprotein_gene(AF052439)_16mers_0MM.txt | YFV clone HONG3 |
| | | polyprotein gene(AF052439) |
| 650) | YFV_clone_HONG3_polyprotein_gene(AF052439)_16mers_1MM.txt | YFV clone HONG3 |
| | | polyprotein gene(AF052439) |
| 651) | YFV_clone_HONG3_polyprotein_gene(AF052439)_16mers_2MM.txt | YFV clone HONG3 |
| | | polyprotein gene(AF052439) |
| 652) | YFV_clone_HONG3_polyprotein_gene(AF052439)_17mers_0MM.txt | YFV clone HONG3 |
| | | polyprotein gene(AF052439) |
| 653) | YFV_clone_HONG3_polyprotein_gene(AF052439)_17mers_1MM.txt | YFV clone HONG3 |
| | | polyprotein gene(AF052439) |
| 654) | YFV_clone_HONG3_polyprotein_gene(AF052439)_17mers_2MM.txt | YFV clone HONG3 |
| | | polyprotein gene(AF052439) |
| 655) | YFV_clone_HONG3_polyprotein_gene(AF052439)_18mers_0MM.txt | YFV clone HONG3 |
| | | polyprotein gene(AF052439) |
| 656) | YFV_clone_HONG3_polyprotein_gene(AF052439)_18mers_1MM.txt | YFV clone HONG3 |
| | | polyprotein gene(AF052439) |
| 657) | YFV_clone_HONG3_polyprotein_gene(AF052439)_18mers_2MM.txt | YFV clone HONG3 |
| | | polyprotein gene(AF052439) |
| 658) | YFV_clone_HONG8_polyprotein_gene(AF052444)_16mers_0MM.txt | YFV clone HONG8 |
| | | polyprotein gene(AF052444) |
| 659) | YFV_clone_HONG8_polyprotein_gene(AF052444)_16mers_1MM.txt | YFV clone HONG8 |
| | | polyprotein gene(AF052444) |
| 660) | YFV_clone_HONG8_polyprotein_gene(AF052444)_16mers_2MM.txt | YFV clone HONG8 |
| | | polyprotein gene(AF052444) |
| 661) | YFV_clone_HONG8_polyprotein_gene(AF052444)_17mers_0MM.txt | YFV clone HONG8 |
| | | polyprotein gene(AF052444) |
| 662) | YFV_clone_HONG8_polyprotein_gene(AF052444)_17mers_1MM.txt | YFV clone HONG8 |
| | | polyprotein gene(AF052444) |
| 663) | YFV_clone_HONG8_polyprotein_gene(AF052444)_17mers_2MM.txt | YFV clone HONG8 |
| | | polyprotein gene(AF052444) |
| 664) | YFV_clone_HONG8_polyprotein_gene(AF052444)_18mers_0MM.txt | YFV clone HONG8 |
| | | polyprotein gene(AF052444) |
| 665) | YFV_clone_HONG8_polyprotein_gene(AF052444)_18mers_1MM.txt | YFV clone HONG8 |
| | | polyprotein gene(AF052444) |
| 666) | YFV_clone_HONG8_polyprotein_gene(AF052444)_18mers_2MM.txt | YFV clone HONG8 |
| | | polyprotein gene(AF052444) |
| 667) | YFV_clone_HONG9_polyprotein_gene(AF052445)_16mers_0MM.txt | YFV clone HONG9 |
| | | polyprotein gene(AF052445) |
| 668) | YFV_clone_HONG9_polyprotein_gene(AF052445)_16mers_1MM.txt | YFV clone HONG9 |
| | | polyprotein gene(AF052445) |
| 669) | YFV_clone_HONG9_polyprotein_gene(AF052445)_16mers_2MM.txt | YFV clone HONG9 |
| | | polyprotein gene(AF052445) |
| 670) | YFV_clone_HONG9_polyprotein_gene(AF052445)_17mers_0MM.txt | YFV clone HONG9 |
| | | polyprotein gene(AF052445) |
| 671) | YFV_clone_HONG9_polyprotein_gene(AF052445)_17mers_1MM.txt | YFV clone HONG9 |
| | | polyprotein gene(AF052445) |
| 672) | YFV_clone_HONG9_polyprotein_gene(AF052445)_17mers_2MM.txt | YFV clone HONG9 |
| | | polyprotein gene(AF052445) |
| 673) | YFV_clone_HONG9_polyprotein_gene(AF052445)_18mers_0MM.txt | YFV clone HONG9 |
| | | polyprotein gene(AF052445) |
| 674) | YFV_clone_HONG9_polyprotein_gene(AF052445)_18mers_1MM.txt | YFV clone HONG9 |
| | | polyprotein gene(AF052445) |
| 675) | YFV_clone_HONG9_polyprotein_gene(AF052445)_18mers_2MM.txt | YFV clone HONG9 |
| | | polyprotein gene(AF052445) |
| 676) | YFV_French_neurotropic_strain(U21055)_16mers_0MM.txt | YFV French neurotropic |
| | | strain(U21055) |
| 677) | YFV_French_neurotropic_strain(U21055)_16mers_1MM.txt | YFV French neurotropic |
| | | strain(U21055) |
| 678) | YFV_French_neurotropic_strain(U21055)_16mers_2MM.txt | YFV French neurotropic |
| | | strain(U21055) |
| 679) | YFV_French_neurotropic_strain(U21055)_17mers_0MM.txt | YFV French neurotropic |
| | | strain(U21055) |
| 680) | YFV_French_neurotropic_strain(U21055)_17mers_1MM.txt | YFV French neurotropic |
| | | strain(U21055) |
| 681) | YFV_French_neurotropic_strain(U21055)_17mers_2MM.txt | YFV French neurotropic |
| | | strain(U21055) |
| 682) | YFV_French_neurotropic_strain(U21055)_18mers_0MM.txt | YFV French neurotropic |
| | | strain(U21055) |
| 683) | YFV_French_neurotropic_strain(U21055)_18mers_1MM.txt | YFV French neurotropic |
| | | strain(U21055) |
| 684) | YFV_French_neurotropic_strain(U21055)_18mers_2MM.txt | YFV French neurotropic |
| | | strain(U21055) |
| 685) | YFV_French_viscerotropic_strain(U21056)_16mers_0MM.txt | YFV French viscerotropic |
| | | strain(U21056) |
| 686) | YFV_French_viscerotropic_strain(U21056)_16mers_1MM.txt | YFV French viscerotropic |
| | | strain(U21056) |
| 687) | YFV_French_viscerotropic_strain(U21056)_16mers_2MM.txt | YFV French viscerotropic |
| | | strain(U21056) |
| 688) | YFV_French_viscerotropic_strain(U21056)_17mers_0MM.txt | YFV French viscerotropic |
| | | strain(U21056) |
| 689) | YFV_French_viscerotropic_strain(U21056)_17mers_1MM.txt | YFV French viscerotropic |
| | | strain(U21056) |
| 690) | YFV_French_viscerotropic_strain(U21056)_17mers_2MM.txt | YFV French viscerotropic |
| | | strain(U21056) |
| 691) | YFV_French_viscerotropic_strain(U21056)_18mers_0MM.txt | YFV French viscerotropic |
| | | strain(U21056) |
| 692) | YFV_French_viscerotropic_strain(U21056)_18mers_1MM.txt | YFV French viscerotropic |
| | | strain(U21056) |
| 693) | YFV_French_viscerotropic_strain(U21056)_18mers_2MM.txt | YFV French viscerotropic |
| | | strain(U21056) |
| 694) | YFV_genomic_RNA(X15062)_16mers_0MM.txt | YFV genomic RNA(X15062) |
| 695) | YFV_genomic_RNA(X15062)_16mers_1MM.txt | YFV genomic RNA(X15062) |
| 696) | YFV_genomic_RNA(X15062)_16mers_2MM.txt | YFV genomic RNA(X15062) |
| 697) | YFV_genomic_RNA(X15062)_17mers_0MM.txt | YFV genomic RNA(X15062) |
| 698) | YFV_genomic_RNA(X15062)_17mers_1MM.txt | YFV genomic RNA(X15062) |
| 699) | YFV_genomic_RNA(X15062)_17mers_2MM.txt | YFV genomic RNA(X15062) |
| 700) | YFV_genomic_RNA(X15062)_18mers_0MM.txt | YFV genomic RNA(X15062) |
| 701) | YFV_genomic_RNA(X15062)_18mers_1MM.txt | YFV genomic RNA(X15062) |
| 702) | YFV_genomic_RNA(X15062)_18mers_2MM.txt | YFV genomic RNA(X15062) |
| 703) | YFV_strain_85-82H_Ivory_Coast(U54798)_16mers_0MM.txt | YFV strain 85-82H Ivory |
| | | Coast(U54798) |
| 704) | YFV_strain_85-82H_Ivory_Coast(U54798)_16mers_1MM.txt | YFV strain 85-82H Ivory |
| | | Coast(U54798) |
| 705) | YFV_strain_85-82H_Ivory_Coast(U54798)_16mers_2MM.txt | YFV strain 85-82H Ivory |
| | | Coast(U54798) |
| 706) | YFV_strain_85-82H_Ivory_Coast(U54798)_17mers_0MM.txt | YFV strain 85-82H Ivory |
| | | Coast(U54798) |
| 707) | YFV_strain_85-82H_Ivory_Coast(U54798)_17mers_1MM.txt | YFV strain 85-82H Ivory |
| | | Coast(U54798) |
| 708) | YFV_strain_85-82H_Ivory_Coast(U54798)_17mers_2MM.txt | YFV strain 85-82H Ivory |
| | | Coast(U54798) |
| 709) | YFV_strain_85-82H_Ivory_Coast(U54798)_18mers_0MM.txt | YFV strain 85-82H Ivory |
| | | Coast(U54798) |
| 710) | YFV_strain_85-82H_Ivory_Coast(U54798)_18mers_1MM.txt | YFV strain 85-82H Ivory |
| | | Coast(U54798) |
| 711) | YFV_strain_85-82H_Ivory_Coast(U54798)_18mers_2MM.txt | YFV strain 85-82H Ivory |
| | | Coast(U54798) |
| 712) | YFV_strain_Gambia_2001(AY572535)_16mers_0MM.txt | YFV strain Gambia |
| | | 2001(AY572535) |
| 713) | YFV_strain_Gambia_2001(AY572535)_16mers_1MM.txt | YFV strain Gambia |
| | | 2001(AY572535) |
| 714) | YFV_strain_Gambia_2001(AY572535)_16mers_2MM.txt | YFV strain Gambia |
| | | 2001(AY572535) |
| 715) | YFV_strain_Gambia_2001(AY572535)_17mers_0MM.txt | YFV strain Gambia |
| | | 2001(AY572535) |
| 716) | YFV_strain_Gambia_2001(AY572535)_17mers_1MM.txt | YFV strain Gambia |
| | | 2001(AY572535) |
| 717) | YFV_strain_Gambia_2001(AY572535)_17mers_2MM.txt | YFV strain Gambia |
| | | 2001(AY572535) |
| 718) | YFV_strain_Gambia_2001(AY572535)_18mers_0MM.txt | YFV strain Gambia |
| | | 2001(AY572535) |
| 719) | YFV_strain_Gambia_2001(AY572535)_18mers_1MM.txt | YFV strain Gambia |
| | | 2001(AY572535) |
| 720) | YFV_strain_Gambia_2001(AY572535)_18mers_2MM.txt | YFV strain Gambia |
| | | 2001(AY572535) |
| 721) | YFV_strain_Ivory_Coast_1999(AY603338)_16mers_0MM.txt | YFV strain Ivory Coast |
| | | 1999(AY603338) |
| 722) | YFV_strain_Ivory_Coast_1999(AY603338)_16mers_1MM.txt | YFV strain Ivory Coast |
| | | 1999(AY603338) |
| 723) | YFV_strain_Ivory_Coast_1999(AY603338)_16mers_2MM.txt | YFV strain Ivory Coast |
| | | 1999(AY603338) |
| 724) | YFV_strain_Ivory_Coast_1999(AY603338)_17mers_0MM.txt | YFV strain Ivory Coast |
| | | 1999(AY603338) |
| 725) | YFV_strain_Ivory_Coast_1999(AY603338)_17mers_1MM.txt | YFV strain Ivory Coast |
| | | 1999(AY603338) |
| 726) | YFV_strain_Ivory_Coast_1999(AY603338)_17mers_2MM.txt | YFV strain Ivory Coast |
| | | 1999(AY603338) |
| 727) | YFV_strain_Ivory_Coast_1999(AY603338)_18mers_0MM.txt | YFV strain Ivory Coast |
| | | 1999(AY603338) |
| 728) | YFV_strain_Ivory_Coast_1999(AY603338)_18mers_1MM.txt | YFV strain Ivory Coast |
| | | 1999(AY603338) |
| 729) | YFV_strain_Ivory_Coast_1999(AY603338)_18mers_2MM.txt | YFV strain Ivory Coast |
| | | 1999(AY603338) |
| 730) | YFV_strain_Trinidad_79A_isolate_788379(AF094612)_16mers_0MM.txt | YFV strain Trinidad 79A |
| | | isolate 788379(AF094612) |
| 731) | YFV_strain_Trinidad_79A_isolate_788379(AF094612)_16mers_1MM.txt | YFV strain Trinidad 79A |
| | | isolate 788379(AF094612) |
| 732) | YFV_strain_Trinidad_79A_isolate_788379(AF094612)_16mers_2MM.txt | YFV strain Trinidad 79A |
| | | isolate 788379(AF094612) |
| 733) | YFV_strain_Trinidad_79A_isolate_788379(AF094612)_17mers_0MM.txt | YFV strain Trinidad 79A |
| | | isolate 788379(AF094612) |
| 734) | YFV_strain_Trinidad_79A_isolate_788379(AF094612)_17mers_1MM.txt | YFV strain Trinidad 79A |
| | | isolate 788379(AF094612) |
| 735) | YFV_strain_Trinidad_79A_isolate_788379(AF094612)_17mers_2MM.txt | YFV strain Trinidad 79A |
| | | isolate 788379(AF094612) |
| 736) | YFV_strain_Trinidad_79A_isolate_788379(AF094612)_18mers_0MM.txt | YFV strain Trinidad 79A |
| | | isolate 788379(AF094612) |
| 737) | YFV_strain_Trinidad_79A_isolate_788379(AF094612)_18mers_1MM.txt | YFV strain Trinidad 79A |
| | | isolate 788379(AF094612) |
| 738) | YFV_strain_Trinidad_79A_isolate_788379(AF094612)_18mers_2MM.txt | YFV strain Trinidad 79A |
| | | isolate 788379(AF094612) |
| 739) | YFV_vaccine_strain_17D-213(U17067)_16mers_0MM.txt | YFV vaccine strain 17D- |
| | | 213(U17067) |
| 740) | YFV_vaccine_strain_17D-213(U17067)_16mers_1MM.txt | YFV vaccine strain 17D- |
| | | 213(U17067) |
| 741) | YFV_vaccine_strain_17D-213(U17067)_16mers_2MM.txt | YFV vaccine strain 17D- |
| | | 213(U17067) |
| 742) | YFV_vaccine_strain_17D-213(U17067)_17mers_0MM.txt | YFV vaccine strain 17D- |
| | | 213(U17067) |
| 743) | YFV_vaccine_strain_17D-213(U17067)_17mers_1MM.txt | YFV vaccine strain 17D- |
| | | 213(U17067) |
| 744) | YFV_vaccine_strain_17D-213(U17067)_17mers_2MM.txt | YFV vaccine strain 17D- |
| | | 213(U17067) |
| 745) | YFV_vaccine_strain_17D-213(U17067)_18mers_0MM.txt | YFV vaccine strain 17D- |
| | | 213(U17067) |
| 746) | YFV_vaccine_strain_17D-213(U17067)_18mers_1MM.txt | YFV vaccine strain 17D- |
| | | 213(U17067) |
| 747) | YFV_vaccine_strain_17D-213(U17067)_18mers_2MM.txt | YFV vaccine strain 17D- |
| | | 213(U17067) |
| 748) | YFV_vaccine_strain_17DD(U17066)_16mers_0MM.txt | YFV vaccine strain 17DD(U17066) |
| 749) | YFV_vaccine_strain_17DD(U17066)_16mers_1MM.txt | YFV vaccine strain 17DD(U17066) |
| 750) | YFV_vaccine_strain_17DD(U17066)_16mers_2MM.txt | YFV vaccine strain 17DD(U17066) |
| 751) | YFV_vaccine_strain_17DD(U17066)_17mers_0MM.txt | YFV vaccine strain 17DD(U17066) |
| 752) | YFV_vaccine_strain_17DD(U17066)_17mers_1MM.txt | YFV vaccine strain 17DD(U17066) |
| 753) | YFV_vaccine_strain_17DD(U17066)_17mers_2MM.txt | YFV vaccine strain 17DD(U17066) |
| 754) | YFV_vaccine_strain_17DD(U17066)_18mers_0MM.txt | YFV vaccine strain 17DD(U17066) |
| 755) | YFV_vaccine_strain_17DD(U17066)_18mers_1MM.txt | YFV vaccine strain 17DD(U17066) |
| 756) | YFV_vaccine_strain_17DD(U17066)_18mers_2MM.txt | YFV vaccine strain 17DD(U17066) |
| 757) | Yokose(NC_005039)_16mers_0MM.txt | Yokose (NC_005039) |
| 758) | Yokose(NC_005039)_16mers_1MM.txt | Yokose (NC_005039) |
| 759) | Yokose(NC_005039)_16mers_2MM.txt | Yokose (NC_005039) |
| 760) | Yokose(NC_005039)_17mers_0MM.txt | Yokose (NC_005039) |
| 761) | Yokose(NC_005039)_17mers_1MM.txt | Yokose (NC_005039) |
| 762) | Yokose(NC_005039)_17mers_2MM.txt | Yokose (NC_005039) |
| 763) | Yokose(NC_005039)_18mers_0MM.txt | Yokose (NC_005039) |
| 764) | Yokose(NC_005039)_18mers_1MM.txt | Yokose (NC_005039) |
| 765) | Yokose(NC_005039)_18mers_2MM.txt | Yokose (NC_005039) |

Folder nmerseqsDV - It contains 1815 Dengue virus human-blind files, containing
sequences which are present in Dengue virus genomes but are not present in Human
genome even with two mismatches. The list below gives us the file name and the
corresponding genome name.

| | | |
|---|---|---|
| 1) | dengu1_19mers_0MM.txt | Dengue type 3 virus complete genome RNA (DENCME) |
| 2) | dengu1_19mers_1MM.txt | Dengue type 3 virus complete genome RNA (DENCME) |
| 3) | dengu1_19mers_2MM.txt | Dengue type 3 virus complete genome RNA (DENCME) |
| 4) | dengu1_20mers_0MM.txt | Dengue type 3 virus complete genome RNA (DENCME) |
| 5) | dengu1_20mers_1MM.txt | Dengue type 3 virus complete genome RNA (DENCME) |
| 6) | dengu1_20mers_2MM.txt | Dengue type 3 virus complete genome RNA (DENCME) |
| 7) | dengu1_21mers_0MM.txt | Dengue type 3 virus complete genome RNA (DENCME) |
| 8) | dengu1_21mers_1MM.txt | Dengue type 3 virus complete genome RNA (DENCME) |
| 9) | dengu1_21mers_2MM.txt | Dengue type 3 virus complete genome RNA (DENCME) |
| 10) | dengu1_22mers_0MM.txt | Dengue type 3 virus complete genome RNA (DENCME) |
| 11) | dengu1_22mers_1MM.txt | Dengue type 3 virus complete genome RNA (DENCME) |
| 12) | dengu1_22mers_2MM.txt | Dengue type 3 virus complete genome RNA (DENCME) |
| 13) | dengu10_19mers_0MM.txt | Dengue virus type 2 strain K0010 polyprotein gene (AF100460) |
| 14) | dengu10_19mers_1MM.txt | Dengue virus type 2 strain K0010 polyprotein gene (AF100460) |
| 15) | dengu10_19mers_2MM.txt | Dengue virus type 2 strain K0010 polyprotein gene (AF100460) |
| 16) | dengu10_20mers_0MM.txt | Dengue virus type 2 strain K0010 polyprotein gene (AF100460) |
| 17) | dengu10_20mers_1MM.txt | Dengue virus type 2 strain K0010 polyprotein gene (AF100460) |
| 18) | dengu10_20mers_2MM.txt | Dengue virus type 2 strain K0010 polyprotein gene (AF100460) |
| 19) | dengu10_21mers_0MM.txt | Dengue virus type 2 strain K0010 polyprotein gene (AF100460) |
| 20) | dengu10_21mers_1MM.txt | Dengue virus type 2 strain K0010 polyprotein gene (AF100460) |
| 21) | dengu10_21mers_2MM.txt | Dengue virus type 2 strain K0010 polyprotein gene (AF100460) |
| 22) | dengu10_22mers_0MM.txt | Dengue virus type 2 strain K0010 polyprotein gene (AF100460) |
| 23) | dengu10_22mers_1MM.txt | Dengue virus type 2 strain K0010 polyprotein gene (AF100460) |
| 24) | dengu10_22mers_2MM.txt | Dengue virus type 2 strain K0010 polyprotein gene (AF100460) |
| 25) | dengu11_19mers_0MM.txt | Dengue virus type 2 strain C0371 polyprotein gene (AF100461) |
| 26) | dengu11_19mers_1MM.txt | Dengue virus type 2 strain C0371 polyprotein gene (AF100461) |
| 27) | dengu11_19mers_2MM.txt | Dengue virus type 2 strain C0371 polyprotein gene (AF100461) |
| 28) | dengu11_20mers_0MM.txt | Dengue virus type 2 strain C0371 polyprotein gene (AF100461) |
| 29) | dengu11_20mers_1MM.txt | Dengue virus type 2 strain C0371 polyprotein gene (AF100461) |
| 30) | dengu11_20mers_2MM.txt | Dengue virus type 2 strain C0371 polyprotein gene (AF100461) |
| 31) | dengu11_21mers_0MM.txt | Dengue virus type 2 strain C0371 polyprotein gene (AF100461) |
| 32) | dengu11_21mers_1MM.txt | Dengue virus type 2 strain C0371 polyprotein gene (AF100461) |
| 33) | dengu11_21mers_2MM.txt | Dengue virus type 2 strain C0371 polyprotein gene (AF100461) |
| 34) | dengu11_22mers_0MM.txt | Dengue virus type 2 strain C0371 polyprotein gene (AF100461) |
| 35) | dengu11_22mers_1MM.txt | Dengue virus type 2 strain C0371 polyprotein gene (AF100461) |
| 36) | dengu11_22mers_2MM.txt | Dengue virus type 2 strain C0371 polyprotein gene (AF100461) |
| 37) | dengu12_19mers_0MM.txt | Dengue virus type 2 strain C0390 polyprotein gene (AF100462) |
| 38) | dengu12_19mers_1MM.txt | Dengue virus type 2 strain C0390 polyprotein gene (AF100462) |
| 39) | dengu12_19mers_2MM.txt | Dengue virus type 2 strain C0390 polyprotein gene (AF100462) |
| 40) | dengu12_20mers_0MM.txt | Dengue virus type 2 strain C0390 polyprotein gene (AF100462) |
| 41) | dengu12_20mers_1MM.txt | Dengue virus type 2 strain C0390 polyprotein gene (AF100462) |
| 42) | dengu12_20mers_2MM.txt | Dengue virus type 2 strain C0390 polyprotein gene (AF100462) |
| 43) | dengu12_21mers_0MM.txt | Dengue virus type 2 strain C0390 polyprotein gene (AF100462) |
| 44) | dengu12_21mers_1MM.txt | Dengue virus type 2 strain C0390 polyprotein gene (AF100462) |
| 45) | dengu12_21mers_2MM.txt | Dengue virus type 2 strain C0390 polyprotein gene (AF100462) |
| 46) | dengu12_22mers_0MM.txt | Dengue virus type 2 strain C0390 polyprotein gene (AF100462) |
| 47) | dengu12_22mers_1MM.txt | Dengue virus type 2 strain C0390 polyprotein gene (AF100462) |
| 48) | dengu12_22mers_2MM.txt | Dengue virus type 2 strain C0390 polyprotein gene (AF100462) |
| 49) | dengu13_19mers_0MM.txt | Dengue virus type 2 strain C0166 polyprotein gene (AF100463) |
| 50) | dengu13_19mers_1MM.txt | Dengue virus type 2 strain C0166 polyprotein gene (AF100463) |
| 51) | dengu13_19mers_2MM.txt | Dengue virus type 2 strain C0166 polyprotein gene (AF100463) |
| 52) | dengu13_20mers_0MM.txt | Dengue virus type 2 strain C0166 polyprotein gene (AF100463) |
| 53) | dengu13_20mers_1MM.txt | Dengue virus type 2 strain C0166 polyprotein gene (AF100463) |
| 54) | dengu13_20mers_2MM.txt | Dengue virus type 2 strain C0166 polyprotein gene (AF100463) |
| 55) | dengu13_21mers_0MM.txt | Dengue virus type 2 strain C0166 polyprotein gene (AF100463) |
| 56) | dengu13_21mers_1MM.txt | Dengue virus type 2 strain C0166 polyprotein gene (AF100463) |
| 57) | dengu13_21mers_2MM.txt | Dengue virus type 2 strain C0166 polyprotein gene (AF100463) |
| 58) | dengu13_22mers_0MM.txt | Dengue virus type 2 strain C0166 polyprotein gene (AF100463) |
| 59) | dengu13_22mers_1MM.txt | Dengue virus type 2 strain C0166 polyprotein gene (AF100463) |
| 60) | dengu13_22mers_2MM.txt | Dengue virus type 2 strain C0166 polyprotein gene (AF100463) |
| 61) | dengu14_19mers_0MM.txt | Dengue virus type 2 strain C0167 polyprotein gene (AF100464) |
| 62) | dengu14_19mers_1MM.txt | Dengue virus type 2 strain C0167 polyprotein gene (AF100464) |
| 63) | dengu14_19mers_2MM.txt | Dengue virus type 2 strain C0167 polyprotein gene (AF100464) |
| 64) | dengu14_20mers_0MM.txt | Dengue virus type 2 strain C0167 polyprotein gene (AF100464) |
| 65) | dengu14_20mers_1MM.txt | Dengue virus type 2 strain C0167 polyprotein gene (AF100464) |
| 66) | dengu14_20mers_2MM.txt | Dengue virus type 2 strain C0167 polyprotein gene (AF100464) |
| 67) | dengu14_21mers_0MM.txt | Dengue virus type 2 strain C0167 polyprotein gene (AF100464) |
| 68) | dengu14_21mers_1MM.txt | Dengue virus type 2 strain C0167 polyprotein gene (AF100464) |
| 69) | dengu14_21mers_2MM.txt | Dengue virus type 2 strain C0167 polyprotein gene (AF100464) |
| 70) | dengu14_22mers_0MM.txt | Dengue virus type 2 strain C0167 polyprotein gene (AF100464) |
| 71) | dengu14_22mers_1MM.txt | Dengue virus type 2 strain C0167 polyprotein gene (AF100464) |
| 72) | dengu14_22mers_2MM.txt | Dengue virus type 2 strain C0167 polyprotein gene (AF100464) |
| 73) | dengu15_19mers_0MM.txt | Dengue virus type 2 strain Ven2 polyprotein gene (AF100465) |
| 74) | dengu15_19mers_1MM.txt | Dengue virus type 2 strain Ven2 polyprotein gene (AF100465) |
| 75) | dengu15_19mers_2MM.txt | Dengue virus type 2 strain Ven2 polyprotein gene (AF100465) |
| 76) | dengu15_20mers_0MM.txt | Dengue virus type 2 strain Ven2 polyprotein gene (AF100465) |
| 77) | dengu15_20mers_1MM.txt | Dengue virus type 2 strain Ven2 polyprotein gene (AF100465) |
| 78) | dengu15_20mers_2MM.txt | Dengue virus type 2 strain Ven2 polyprotein gene (AF100465) |
| 79) | dengu15_21mers_0MM.txt | Dengue virus type 2 strain Ven2 polyprotein gene (AF100465) |
| 80) | dengu15_21mers_1MM.txt | Dengue virus type 2 strain Ven2 polyprotein gene (AF100465) |
| 81) | dengu15_21mers_2MM.txt | Dengue virus type 2 strain Ven2 polyprotein gene (AF100465) |
| 82) | dengu15_22mers_0MM.txt | Dengue virus type 2 strain Ven2 polyprotein gene (AF100465) |
| 83) | dengu15_22mers_1MM.txt | Dengue virus type 2 strain Ven2 polyprotein gene (AF100465) |
| 84) | dengu15_22mers_2MM.txt | Dengue virus type 2 strain Ven2 polyprotein gene (AF100465) |
| 85) | dengu16_19mers_0MM.txt | Dengue virus type 2 strain IQT1797 polyprotein gene (AF100467) |
| 86) | dengu16_19mers_1MM.txt | Dengue virus type 2 strain IQT1797 polyprotein gene (AF100467) |
| 87) | dengu16_19mers_2MM.txt | Dengue virus type 2 strain IQT1797 polyprotein gene (AF100467) |
| 88) | dengu16_20mers_0MM.txt | Dengue virus type 2 strain IQT1797 polyprotein gene (AF100467) |
| 89) | dengu16_20mers_1MM.txt | Dengue virus type 2 strain IQT1797 polyprotein gene (AF100467) |
| 90) | dengu16_20mers_2MM.txt | Dengue virus type 2 strain IQT1797 polyprotein gene (AF100467) |
| 91) | dengu16_21mers_0MM.txt | Dengue virus type 2 strain IQT1797 polyprotein gene (AF100467) |
| 92) | dengu16_21mers_1MM.txt | Dengue virus type 2 strain IQT1797 polyprotein gene (AF100467) |
| 93) | dengu16_21mers_2MM.txt | Dengue virus type 2 strain IQT1797 polyprotein gene (AF100467) |
| 94) | dengu16_22mers_0MM.txt | Dengue virus type 2 strain IQT1797 polyprotein gene (AF100467) |
| 95) | dengu16_22mers_1MM.txt | Dengue virus type 2 strain IQT1797 polyprotein gene (AF100467) |
| 96) | dengu16_22mers_2MM.txt | Dengue virus type 2 strain IQT1797 polyprotein gene (AF100467) |
| 97) | dengu17_19mers_0MM.txt | Dengue virus type 2 strain IQT2913 polyprotein gene (AF100468) |
| 98) | dengu17_19mers_1MM.txt | Dengue virus type 2 strain IQT2913 polyprotein gene (AF100468) |
| 99) | dengu17_19mers_2MM.txt | Dengue virus type 2 strain IQT2913 polyprotein gene (AF100468) |
| 100) | dengu17_20mers_0MM.txt | Dengue virus type 2 strain IQT2913 polyprotein gene (AF100468) |
| 101) | dengu17_20mers_1MM.txt | Dengue virus type 2 strain IQT2913 polyprotein gene (AF100468) |
| 102) | dengu17_20mers_2MM.txt | Dengue virus type 2 strain IQT2913 polyprotein gene (AF100468) |
| 103) | dengu17_21mers_0MM.txt | Dengue virus type 2 strain IQT2913 polyprotein gene (AF100468) |
| 104) | dengu17_21mers_1MM.txt | Dengue virus type 2 strain IQT2913 polyprotein gene (AF100468) |
| 105) | dengu17_21mers_2MM.txt | Dengue virus type 2 strain IQT2913 polyprotein gene (AF100468) |
| 106) | dengu17_22mers_0MM.txt | Dengue virus type 2 strain IQT2913 polyprotein gene (AF100468) |
| 107) | dengu17_22mers_1MM.txt | Dengue virus type 2 strain IQT2913 polyprotein gene (AF100468) |
| 108) | dengu17_22mers_2MM.txt | Dengue virus type 2 strain IQT2913 polyprotein gene (AF100468) |
| 109) | dengu18_19mers_0MM.txt | Dengue virus type 2 strain 131 polyprotein gene (AF100469) |
| 110) | dengu18_19mers_1MM.txt | Dengue virus type 2 strain 131 polyprotein gene (AF100469) |
| 111) | dengu18_19mers_2MM.txt | Dengue virus type 2 strain 131 polyprotein gene (AF100469) |
| 112) | dengu18_20mers_0MM.txt | Dengue virus type 2 strain 131 polyprotein gene (AF100469) |
| 113) | dengu18_20mers_1MM.txt | Dengue virus type 2 strain 131 polyprotein gene (AF100469) |
| 114) | dengu18_20mers_2MM.txt | Dengue virus type 2 strain 131 polyprotein gene (AF100469) |
| 115) | dengu18_21mers_0MM.txt | Dengue virus type 2 strain 131 polyprotein gene (AF100469) |
| 116) | dengu18_21mers_1MM.txt | Dengue virus type 2 strain 131 polyprotein gene (AF100469) |
| 117) | dengu18_21mers_2MM.txt | Dengue virus type 2 strain 131 polyprotein gene (AF100469) |
| 118) | dengu18_22mers_0MM.txt | Dengue virus type 2 strain 131 polyprotein gene (AF100469) |
| 119) | dengu18_22mers_1MM.txt | Dengue virus type 2 strain 131 polyprotein gene (AF100469) |
| 120) | dengu18_22mers_2MM.txt | Dengue virus type 2 strain 131 polyprotein gene (AF100469) |
| 121) | dengu2_19mers_0MM.txt | Dengue virus type 2 strain 16681-PDK53 capsid protein (C) gene |
| | | (DENCMEMSA) |
| 122) | dengu2_19mers_1MM.txt | Dengue virus type 2 strain 16681-PDK53 capsid protein (C) gene |
| | | (DENCMEMSA) |
| 123) | dengu2_19mers_2MM.txt | Dengue virus type 2 strain 16681-PDK53 capsid protein (C) gene |
| | | (DENCMEMSA) |
| 124) | dengu2_20mers_0MM.txt | Dengue virus type 2 strain 16681-PDK53 capsid protein (C) gene |
| | | (DENCMEMSA) |
| 125) | dengu2_20mers_1MM.txt | Dengue virus type 2 strain 16681-PDK53 capsid protein (C) gene |
| | | (DENCMEMSA) |
| 126) | dengu2_20mers_2MM.txt | Dengue virus type 2 strain 16681-PDK53 capsid protein (C) gene |
| | | (DENCMEMSA) |
| 127) | dengu2_21mers_0MM.txt | Dengue virus type 2 strain 16681-PDK53 capsid protein (C) gene |
| | | (DENCMEMSA) |
| 128) | dengu2_21mers_1MM.txt | Dengue virus type 2 strain 16681-PDK53 capsid protein (C) gene |
| | | (DENCMEMSA) |
| 129) | dengu2_21mers_2MM.txt | Dengue virus type 2 strain 16681-PDK53 capsid protein (C) gene |
| | | (DENCMEMSA) |
| 130) | dengu2_22mers_0MM.txt | Dengue virus type 2 strain 16681-PDK53 capsid protein (C) gene |
| | | (DENCMEMSA) |
| 131) | dengu2_22mers_1MM.txt | Dengue virus type 2 strain 16681-PDK53 capsid protein (C) gene |
| | | (DENCMEMSA) |
| 132) | dengu2_22mers_2MM.txt | Dengue virus type 2 strain 16681-PDK53 capsid protein (C) gene |
| | | (DENCMEMSA) |
| 133) | dengu20_19mers_0MM.txt | Dengue virus type 4 polyprotein precursor (DENSTRA) |
| 134) | dengu20_19mers_1MM.txt | Dengue virus type 4 polyprotein precursor (DENSTRA) |
| 135) | dengu20_19mers_2MM.txt | Dengue virus type 4 polyprotein precursor (DENSTRA) |
| 136) | dengu20_20mers_0MM.txt | Dengue virus type 4 polyprotein precursor (DENSTRA) |
| 137) | dengu20_20mers_1MM.txt | Dengue virus type 4 polyprotein precursor (DENSTRA) |
| 138) | dengu20_20mers_2MM.txt | Dengue virus type 4 polyprotein precursor (DENSTRA) |
| 139) | dengu20_21mers_0MM.txt | Dengue virus type 4 polyprotein precursor (DENSTRA) |
| 140) | dengu20_21mers_1MM.txt | Dengue virus type 4 polyprotein precursor (DENSTRA) |
| 141) | dengu20_21mers_2MM.txt | Dengue virus type 4 polyprotein precursor (DENSTRA) |
| 142) | dengu20_22mers_0MM.txt | Dengue virus type 4 polyprotein precursor (DENSTRA) |
| 143) | dengu20_22mers_1MM.txt | Dengue virus type 4 polyprotein precursor (DENSTRA) |
| 144) | dengu20_22mers_2MM.txt | Dengue virus type 4 polyprotein precursor (DENSTRA) |
| 145) | dengu21_19mers_0MM.txt | Dengue virus type 1 strain 16007 polyprotein precursor (AF180817) |
| 146) | dengu21_19mers_1MM.txt | Dengue virus type 1 strain 16007 polyprotein precursor (AF180817) |
| 147) | dengu21_19mers_2MM.txt | Dengue virus type 1 strain 16007 polyprotein precursor (AF180817) |
| 148) | dengu21_20mers_0MM.txt | Dengue virus type 1 strain 16007 polyprotein precursor (AF180817) |
| 149) | dengu21_20mers_1MM.txt | Dengue virus type 1 strain 16007 polyprotein precursor (AF180817) |
| 150) | dengu21_20mers_2MM.txt | Dengue virus type 1 strain 16007 polyprotein precursor (AF180817) |
| 151) | dengu21_21mers_0MM.txt | Dengue virus type 1 strain 16007 polyprotein precursor (AF180817) |
| 152) | dengu21_21mers_1MM.txt | Dengue virus type 1 strain 16007 polyprotein precursor (AF180817) |
| 153) | dengu21_21mers_2MM.txt | Dengue virus type 1 strain 16007 polyprotein precursor (AF180817) |
| 154) | dengu21_22mers_0MM.txt | Dengue virus type 1 strain 16007 polyprotein precursor (AF180817) |
| 155) | dengu21_22mers_1MM.txt | Dengue virus type 1 strain 16007 polyprotein precursor (AF180817) |
| 156) | dengu21_22mers_2MM.txt | Dengue virus type 1 strain 16007 polyprotein precursor (AF180817) |
| 157) | dengu22_19mers_0MM.txt | Dengue virus type 1 strain 16007 (PDK-13) polyprotein precursor |
| | | (AF180818) |
| 158) | dengu22_19mers_1MM.txt | Dengue virus type 1 strain 16007 (PDK-13) polyprotein precursor |
| | | (AF180818) |
| 159) | dengu22_19mers_2MM.txt | Dengue virus type 1 strain 16007 (PDK-13) polyprotein precursor |
| | | (AF180818) |
| 160) | dengu22_20mers_0MM.txt | Dengue virus type 1 strain 16007 (PDK-13) polyprotein precursor |
| | | (AF180818) |
| 161) | dengu22_20mers_1MM.txt | Dengue virus type 1 strain 16007 (PDK-13) polyprotein precursor |
| | | (AF180818) |
| 162) | dengu22_20mers_2MM.txt | Dengue virus type 1 strain 16007 (PDK-13) polyprotein precursor |
| | | (AF180818) |
| 163) | dengu22_21mers_0MM.txt | Dengue virus type 1 strain 16007 (PDK-13) polyprotein precursor |
| | | (AF180818) |
| 164) | dengu22_21mers_1MM.txt | Dengue virus type 1 strain 16007 (PDK-13) polyprotein precursor |
| | | (AF180818) |
| 165) | dengu22_21mers_2MM.txt | Dengue virus type 1 strain 16007 (PDK-13) polyprotein precursor |
| | | (AF180818) |
| 166) | dengu22_22mers_0MM.txt | Dengue virus type 1 strain 16007 (PDK-13) polyprotein precursor |
| | | (AF180818) |
| 167) | dengu22_22mers_1MM.txt | Dengue virus type 1 strain 16007 (PDK-13) polyprotein precursor |
| | | (AF180818) |
| 168) | dengu22_22mers_2MM.txt | Dengue virus type 1 strain 16007 (PDK-13) polyprotein precursor |
| | | (AF180818) |
| 169) | dengu23_19mers_0MM.txt | dengue virus type 4 China Guangzhou B5 strain genomic RNA |
| | | (AF289029) |
| 170) | dengu23_19mers_1MM.txt | dengue virus type 4 China Guangzhou B5 strain genomic RNA |
| | | (AF289029) |
| 171) | dengu23_19mers_2MM.txt | dengue virus type 4 China Guangzhou B5 strain genomic RNA |
| | | (AF289029) |
| 172) | dengu23_20mers_0MM.txt | dengue virus type 4 China Guangzhou B5 strain genomic RNA |
| | | (AF289029) |
| 173) | dengu23_20mers_1MM.txt | dengue virus type 4 China Guangzhou B5 strain genomic RNA |
| | | (AF289029) |
| 174) | dengu23_20mers_2MM.txt | dengue virus type 4 China Guangzhou B5 strain genomic RNA |
| | | (AF289029) |
| 175) | dengu23_21mers_0MM.txt | dengue virus type 4 China Guangzhou B5 strain genomic RNA |
| | | (AF289029) |
| 176) | dengu23_21mers_1MM.txt | dengue virus type 4 China Guangzhou B5 strain genomic RNA |
| | | (AF289029) |
| 177) | dengu23_21mers_2MM.txt | dengue virus type 4 China Guangzhou B5 strain genomic RNA |
| | | (AF289029) |
| 178) | dengu23_22mers_0MM.txt | dengue virus type 4 China Guangzhou B5 strain genomic RNA |
| | | (AF289029) |
| 179) | dengu23_22mers_1MM.txt | dengue virus type 4 China Guangzhou B5 strain genomic RNA |
| | | (AF289029) |
| 180) | dengu23_22mers_2MM.txt | dengue virus type 4 China Guangzhou B5 strain genomic RNA |
| | | (AF289029) |
| 181) | dengu24_19mers_0MM.txt | Dengue virus type 2 strain DEN2/H/IMTSSA-MART/98-703 |
| | | (AF208496) |
| 182) | dengu24_19mers_1MM.txt | Dengue virus type 2 strain DEN2/H/IMTSSA-MART/98-703 |
| | | (AF208496) |
| 183) | dengu24_19mers_2MM.txt | Dengue virus type 2 strain DEN2/H/IMTSSA-MART/98-703 |
| | | (AF208496) |
| 184) | dengu24_20mers_0MM.txt | Dengue virus type 2 strain DEN2/H/IMTSSA-MART/98-703 |
| | | (AF208496) |
| 185) | dengu24_20mers_1MM.txt | Dengue virus type 2 strain DEN2/H/IMTSSA-MART/98-703 |
| | | (AF208496) |
| 186) | dengu24_20mers_2MM.txt | Dengue virus type 2 strain DEN2/H/IMTSSA-MART/98-703 |
| | | (AF208496) |
| 187) | dengu24_21mers_0MM.txt | Dengue virus type 2 strain DEN2/H/IMTSSA-MART/98-703 |
| | | (AF208496) |
| 188) | dengu24_21mers_1MM.txt | Dengue virus type 2 strain DEN2/H/IMTSSA-MART/98-703 |
| | | (AF208496) |
| 189) | dengu24_21mers_2MM.txt | Dengue virus type 2 strain DEN2/H/IMTSSA-MART/98-703 |
| | | (AF208496) |
| 190) | dengu24_22mers_0MM.txt | Dengue virus type 2 strain DEN2/H/IMTSSA-MART/98-703 |
| | | (AF208496) |
| 191) | dengu24_22mers_1MM.txt | Dengue virus type 2 strain DEN2/H/IMTSSA-MART/98-703 |
| | | (AF208496) |
| 192) | dengu24_22mers_2MM.txt | Dengue virus type 2 strain DEN2/H/IMTSSA-MART/98-703 |
| | | (AF208496) |
| 193) | dengu25_19mers_0MM.txt | Dengue virus type 4 recombinant clone rDEN4 (AF326825) |
| 194) | dengu25_19mers_1MM.txt | Dengue virus type 4 recombinant clone rDEN4 (AF326825) |
| 195) | dengu25_19mers_2MM.txt | Dengue virus type 4 recombinant clone rDEN4 (AF326825) |
| 196) | dengu25_20mers_0MM.txt | Dengue virus type 4 recombinant clone rDEN4 (AF326825) |
| 197) | dengu25_20mers_1MM.txt | Dengue virus type 4 recombinant clone rDEN4 (AF326825) |
| 198) | dengu25_20mers_2MM.txt | Dengue virus type 4 recombinant clone rDEN4 (AF326825) |
| 199) | dengu25_21mers_0MM.txt | Dengue virus type 4 recombinant clone rDEN4 (AF326825) |
| 200) | dengu25_21mers_1MM.txt | Dengue virus type 4 recombinant clone rDEN4 (AF326825) |
| 201) | dengu25_21mers_2MM.txt | Dengue virus type 4 recombinant clone rDEN4 (AF326825) |
| 202) | dengu25_22mers_0MM.txt | Dengue virus type 4 recombinant clone rDEN4 (AF326825) |
| 203) | dengu25_22mers_1MM.txt | Dengue virus type 4 recombinant clone rDEN4 (AF326825) |
| 204) | dengu25_22mers_2MM.txt | Dengue virus type 4 recombinant clone rDEN4 (AF326825) |
| 205) | dengu26_19mers_0MM.txt | Dengue virus type 4 recombinant clone 2Adel30 (AF326826) |
| 206) | dengu26_19mers_1MM.txt | Dengue virus type 4 recombinant clone 2Adel30 (AF326826) |
| 207) | dengu26_19mers_2MM.txt | Dengue virus type 4 recombinant clone 2Adel30 (AF326826) |
| 208) | dengu26_20mers_0MM.txt | Dengue virus type 4 recombinant clone 2Adel30 (AF326826) |
| 209) | dengu26_20mers_1MM.txt | Dengue virus type 4 recombinant clone 2Adel30 (AF326826) |
| 210) | dengu26_20mers_2MM.txt | Dengue virus type 4 recombinant clone 2Adel30 (AF326826) |
| 211) | dengu26_21mers_0MM.txt | Dengue virus type 4 recombinant clone 2Adel30 (AF326826) |
| 212) | dengu26_21mers_1MM.txt | Dengue virus type 4 recombinant clone 2Adel30 (AF326826) |
| 213) | dengu26_21mers_2MM.txt | Dengue virus type 4 recombinant clone 2Adel30 (AF326826) |
| 214) | dengu26_22mers_0MM.txt | Dengue virus type 4 recombinant clone 2Adel30 (AF326826) |
| 215) | dengu26_22mers_1MM.txt | Dengue virus type 4 recombinant clone 2Adel30 (AF326826) |
| 216) | dengu26_22mers_2MM.txt | Dengue virus type 4 recombinant clone 2Adel30 (AF326826) |
| 217) | dengu27_19mers_0MM.txt | Dengue virus type 4 recombinant clone rDEN4del30 (AF326827) |
| 218) | dengu27_19mers_1MM.txt | Dengue virus type 4 recombinant clone rDEN4del30 (AF326827) |
| 219) | dengu27_19mers_2MM.txt | Dengue virus type 4 recombinant clone rDEN4del30 (AF326827) |
| 220) | dengu27_20mers_0MM.txt | Dengue virus type 4 recombinant clone rDEN4del30 (AF326827) |
| 221) | dengu27_20mers_1MM.txt | Dengue virus type 4 recombinant clone rDEN4del30 (AF326827) |
| 222) | dengu27_20mers_2MM.txt | Dengue virus type 4 recombinant clone rDEN4del30 (AF326827) |
| 223) | dengu27_21mers_0MM.txt | Dengue virus type 4 recombinant clone rDEN4del30 (AF326827) |
| 224) | dengu27_21mers_1MM.txt | Dengue virus type 4 recombinant clone rDEN4del30 (AF326827) |
| 225) | dengu27_21mers_2MM.txt | Dengue virus type 4 recombinant clone rDEN4del30 (AF326827) |
| 226) | dengu27_22mers_0MM.txt | Dengue virus type 4 recombinant clone rDEN4del30 (AF326827) |
| 227) | dengu27_22mers_1MM.txt | Dengue virus type 4 recombinant clone rDEN4del30 (AF326827) |
| 228) | dengu27_22mers_2MM.txt | Dengue virus type 4 recombinant clone rDEN4del30 (AF326827) |
| 229) | dengu28_19mers_0MM.txt | Dengue virus type 4 strain 814669 (AF326573) |
| 230) | dengu28_19mers_1MM.txt | Dengue virus type 4 strain 814669 (AF326573) |
| 231) | dengu28_19mers_2MM.txt | Dengue virus type 4 strain 814669 (AF326573) |
| 232) | dengu28_20mers_0MM.txt | Dengue virus type 4 strain 814669 (AF326573) |
| 233) | dengu28_20mers_1MM.txt | Dengue virus type 4 strain 814669 (AF326573) |
| 234) | dengu28_20mers_2MM.txt | Dengue virus type 4 strain 814669 (AF326573) |
| 235) | dengu28_21mers_0MM.txt | Dengue virus type 4 strain 814669 (AF326573) |
| 236) | dengu28_21mers_1MM.txt | Dengue virus type 4 strain 814669 (AF326573) |
| 237) | dengu28_21mers_2MM.txt | Dengue virus type 4 strain 814669 (AF326573) |
| 238) | dengu28_22mers_0MM.txt | Dengue virus type 4 strain 814669 (AF326573) |
| 239) | dengu28_22mers_1MM.txt | Dengue virus type 4 strain 814669 (AF326573) |
| 240) | dengu28_22mers_2MM.txt | Dengue virus type 4 strain 814669 (AF326573) |
| 241) | dengu29_19mers_0MM.txt | Dengue virus type 3 strain 80-2 (AF317645) |
| 242) | dengu29_19mers_1MM.txt | Dengue virus type 3 strain 80-2 (AF317645) |
| 243) | dengu29_19mers_2MM.txt | Dengue virus type 3 strain 80-2 (AF317645) |
| 244) | dengu29_20mers_0MM.txt | Dengue virus type 3 strain 80-2 (AF317645) |
| 245) | dengu29_20mers_1MM.txt | Dengue virus type 3 strain 80-2 (AF317645) |
| 246) | dengu29_20mers_2MM.txt | Dengue virus type 3 strain 80-2 (AF317645) |
| 247) | dengu29_21mers_0MM.txt | Dengue virus type 3 strain 80-2 (AF317645) |
| 248) | dengu29_21mers_1MM.txt | Dengue virus type 3 strain 80-2 (AF317645) |
| 249) | dengu29_21mers_2MM.txt | Dengue virus type 3 strain 80-2 (AF317645) |
| 250) | dengu29_22mers_0MM.txt | Dengue virus type 3 strain 80-2 (AF317645) |
| 251) | dengu29_22mers_1MM.txt | Dengue virus type 3 strain 80-2 (AF317645) |
| 252) | dengu29_22mers_2MM.txt | Dengue virus type 3 strain 80-2 (AF317645) |
| 253) | dengu3_19mers_0MM.txt | Dengue virus type 2 strain 16681 capsid protein (C) gene |
| | | (DENCMEMSB) |
| 254) | dengu3_19mers_1MM.txt | Dengue virus type 2 strain 16681 capsid protein (C) gene |
| | | (DENCMEMSB) |
| 255) | dengu3_19mers_2MM.txt | Dengue virus type 2 strain 16681 capsid protein (C) gene |
| | | (DENCMEMSB) |
| 256) | dengu3_20mers_0MM.txt | Dengue virus type 2 strain 16681 capsid protein (C) gene |
| | | (DENCMEMSB) |
| 257) | dengu3_20mers_1MM.txt | Dengue virus type 2 strain 16681 capsid protein (C) gene |
| | | (DENCMEMSB) |
| 258) | dengu3_20mers_2MM.txt | Dengue virus type 2 strain 16681 capsid protein (C) gene |
| | | (DENCMEMSB) |
| 259) | dengu3_21mers_0MM.txt | Dengue virus type 2 strain 16681 capsid protein (C) gene |
| | | (DENCMEMSB) |
| 260) | dengu3_21mers_1MM.txt | Dengue virus type 2 strain 16681 capsid protein (C) gene |
| | | (DENCMEMSB) |
| 261) | dengu3_21mers_2MM.txt | Dengue virus type 2 strain 16681 capsid protein (C) gene |
| | | (DENCMEMSB) |
| 262) | dengu3_22mers_0MM.txt | Dengue virus type 2 strain 16681 capsid protein (C) gene |
| | | (DENCMEMSB) |
| 263) | dengu3_22mers_1MM.txt | Dengue virus type 2 strain 16681 capsid protein (C) gene |
| | | (DENCMEMSB) |
| 264) | dengu3_22mers_2MM.txt | Dengue virus type 2 strain 16681 capsid protein (C) gene |
| | | (DENCMEMSB) |
| 265) | dengu30_19mers_0MM.txt | Dengue virus type 2 strain Mara4 polyprotein gene (AF100466) |
| 266) | dengu30_19mers_1MM.txt | Dengue virus type 2 strain Mara4 polyprotein gene (AF100466) |
| 267) | dengu30_19mers_2MM.txt | Dengue virus type 2 strain Mara4 polyprotein gene (AF100466) |
| 268) | dengu30_20mers_0MM.txt | Dengue virus type 2 strain Mara4 polyprotein gene (AF100466) |
| 269) | dengu30_20mers_1MM.txt | Dengue virus type 2 strain Mara4 polyprotein gene (AF100466) |
| 270) | dengu30_20mers_2MM.txt | Dengue virus type 2 strain Mara4 polyprotein gene (AF100466) |
| 271) | dengu30_21mers_0MM.txt | Dengue virus type 2 strain Mara4 polyprotein gene (AF100466) |
| 272) | dengu30_21mers_1MM.txt | Dengue virus type 2 strain Mara4 polyprotein gene (AF100466) |
| 273) | dengu30_21mers_2MM.txt | Dengue virus type 2 strain Mara4 polyprotein gene (AF100466) |
| 274) | dengu30_22mers_0MM.txt | Dengue virus type 2 strain Mara4 polyprotein gene (AF100466) |
| 275) | dengu30_22mers_1MM.txt | Dengue virus type 2 strain Mara4 polyprotein gene (AF100466) |
| 276) | dengu30_22mers_2MM.txt | Dengue virus type 2 strain Mara4 polyprotein gene (AF100466) |
| 277) | dengu31_19mers_0MM.txt | Dengue virus type 1 strain GZ/80 polyprotein precursor (AF350498) |
| 278) | dengu31_19mers_1MM.txt | Dengue virus type 1 strain GZ/80 polyprotein precursor (AF350498) |
| 279) | dengu31_19mers_2MM.txt | Dengue virus type 1 strain GZ/80 polyprotein precursor (AF350498) |
| 280) | dengu31_20mers_0MM.txt | Dengue virus type 1 strain GZ/80 polyprotein precursor (AF350498) |
| 281) | dengu31_20mers_1MM.txt | Dengue virus type 1 strain GZ/80 polyprotein precursor (AF350498) |
| 282) | dengu31_20mers_2MM.txt | Dengue virus type 1 strain GZ/80 polyprotein precursor (AF350498) |
| 283) | dengu31_21mers_0MM.txt | Dengue virus type 1 strain GZ/80 polyprotein precursor (AF350498) |
| 284) | dengu31_21mers_1MM.txt | Dengue virus type 1 strain GZ/80 polyprotein precursor (AF350498) |
| 285) | dengu31_21mers_2MM.txt | Dengue virus type 1 strain GZ/80 polyprotein precursor (AF350498) |
| 286) | dengu31_22mers_0MM.txt | Dengue virus type 1 strain GZ/80 polyprotein precursor (AF350498) |
| 287) | dengu31_22mers_1MM.txt | Dengue virus type 1 strain GZ/80 polyprotein precursor (AF350498) |
| 288) | dengu31_22mers_2MM.txt | Dengue virus type 1 strain GZ/80 polyprotein precursor (AF350498) |
| 289) | dengu32_19mers_0MM.txt | Dengue virus type 2 strain FJ11/99 (AF359579) |
| 290) | dengu32_19mers_1MM.txt | Dengue virus type 2 strain FJ11/99 (AF359579) |
| 291) | dengu32_19mers_2MM.txt | Dengue virus type 2 strain FJ11/99 (AF359579) |
| 292) | dengu32_20mers_0MM.txt | Dengue virus type 2 strain FJ11/99 (AF359579) |
| 293) | dengu32_20mers_1MM.txt | Dengue virus type 2 strain FJ11/99 (AF359579) |
| 294) | dengu32_20mers_2MM.txt | Dengue virus type 2 strain FJ11/99 (AF359579) |
| 295) | dengu32_21mers_0MM.txt | Dengue virus type 2 strain FJ11/99 (AF359579) |
| 296) | dengu32_21mers_1MM.txt | Dengue virus type 2 strain FJ11/99 (AF359579) |
| 297) | dengu32_21mers_2MM.txt | Dengue virus type 2 strain FJ11/99 (AF359579) |
| 298) | dengu32_22mers_0MM.txt | Dengue virus type 2 strain FJ11/99 (AF359579) |
| 299) | dengu32_22mers_1MM.txt | Dengue virus type 2 strain FJ11/99 (AF359579) |
| 300) | dengu32_22mers_2MM.txt | Dengue virus type 2 strain FJ11/99 (AF359579) |
| 301) | dengu33_19mers_0MM.txt | Dengue virus type 1 strain FGA/89 (AF226687) |
| 302) | dengu33_19mers_1MM.txt | Dengue virus type 1 strain FGA/89 (AF226687) |
| 303) | dengu33_19mers_2MM.txt | Dengue virus type 1 strain FGA/89 (AF226687) |
| 304) | dengu33_20mers_0MM.txt | Dengue virus type 1 strain FGA/89 (AF226687) |
| 305) | dengu33_20mers_1MM.txt | Dengue virus type 1 strain FGA/89 (AF226687) |
| 306) | dengu33_20mers_2MM.txt | Dengue virus type 1 strain FGA/89 (AF226687) |
| 307) | dengu33_21mers_0MM.txt | Dengue virus type 1 strain FGA/89 (AF226687) |
| 308) | dengu33_21mers_1MM.txt | Dengue virus type 1 strain FGA/89 (AF226687) |
| 309) | dengu33_21mers_2MM.txt | Dengue virus type 1 strain FGA/89 (AF226687) |
| 310) | dengu33_22mers_0MM.txt | Dengue virus type 1 strain FGA/89 (AF226687) |
| 311) | dengu33_22mers_1MM.txt | Dengue virus type 1 strain FGA/89 (AF226687) |
| 312) | dengu33_22mers_2MM.txt | Dengue virus type 1 strain FGA/89 (AF226687) |
| 313) | dengu34_19mers_0MM.txt | Dengue virus type 4 recombinant clone 2A (AF375822) |
| 314) | dengu34_19mers_1MM.txt | Dengue virus type 4 recombinant clone 2A (AF375822) |
| 315) | dengu34_19mers_2MM.txt | Dengue virus type 4 recombinant clone 2A (AF375822) |
| 316) | dengu34_20mers_0MM.txt | Dengue virus type 4 recombinant clone 2A (AF375822) |
| 317) | dengu34_20mers_1MM.txt | Dengue virus type 4 recombinant clone 2A (AF375822) |
| 318) | dengu34_20mers_2MM.txt | Dengue virus type 4 recombinant clone 2A (AF375822) |
| 319) | dengu34_21mers_0MM.txt | Dengue virus type 4 recombinant clone 2A (AF375822) |
| 320) | dengu34_21mers_1MM.txt | Dengue virus type 4 recombinant clone 2A (AF375822) |
| 321) | dengu34_21mers_2MM.txt | Dengue virus type 4 recombinant clone 2A (AF375822) |
| 322) | dengu34_22mers_0MM.txt | Dengue virus type 4 recombinant clone 2A (AF375822) |
| 323) | dengu34_22mers_1MM.txt | Dengue virus type 4 recombinant clone 2A (AF375822) |
| 324) | dengu34_22mers_2MM.txt | Dengue virus type 4 recombinant clone 2A (AF375822) |
| 325) | dengu35_19mers_0MM.txt | Dengue virus type 1 Abidjan strain polyprotein gene (AF298807) |
| 326) | dengu35_19mers_1MM.txt | Dengue virus type 1 Abidjan strain polyprotein gene (AF298807) |
| 327) | dengu35_19mers_2MM.txt | Dengue virus type 1 Abidjan strain polyprotein gene (AF298807) |
| 328) | dengu35_20mers_0MM.txt | Dengue virus type 1 Abidjan strain polyprotein gene (AF298807) |
| 329) | dengu35_20mers_1MM.txt | Dengue virus type 1 Abidjan strain polyprotein gene (AF298807) |
| 330) | dengu35_20mers_2MM.txt | Dengue virus type 1 Abidjan strain polyprotein gene (AF298807) |
| 331) | dengu35_21mers_0MM.txt | Dengue virus type 1 Abidjan strain polyprotein gene (AF298807) |
| 332) | dengu35_21mers_1MM.txt | Dengue virus type 1 Abidjan strain polyprotein gene (AF298807) |
| 333) | dengu35_21mers_2MM.txt | Dengue virus type 1 Abidjan strain polyprotein gene (AF298807) |
| 334) | dengu35_22mers_0MM.txt | Dengue virus type 1 Abidjan strain polyprotein gene (AF298807) |
| 335) | dengu35_22mers_1MM.txt | Dengue virus type 1 Abidjan strain polyprotein gene (AF298807) |
| 336) | dengu35_22mers_2MM.txt | Dengue virus type 1 Abidjan strain polyprotein gene (AF298807) |
| 337) | dengu36_19mers_0MM.txt | Dengue virus type 1 Djibouti strain D1/H/IMTSSA/98/606 polyprotein |
| | | (AF298808) |
| 338) | dengu36_19mers_1MM.txt | Dengue virus type 1 Djibouti strain D1/H/IMTSSA/98/606 polyprotein |
| | | (AF298808) |
| 339) | dengu36_19mers_2MM.txt | Dengue virus type 1 Djibouti strain D1/H/IMTSSA/98/606 polyprotein |
| | | (AF298808) |
| 340) | dengu36_20mers_0MM.txt | Dengue virus type 1 Djibouti strain D1/H/IMTSSA/98/606 polyprotein |
| | | (AF298808) |
| 341) | dengu36_20mers_1MM.txt | Dengue virus type 1 Djibouti strain D1/H/IMTSSA/98/606 polyprotein |
| | | (AF298808) |
| 342) | dengu36_20mers_2MM.txt | Dengue virus type 1 Djibouti strain D1/H/IMTSSA/98/606 polyprotein |
| | | (AF298808) |
| 343) | dengu36_21mers_0MM.txt | Dengue virus type 1 Djibouti strain D1/H/IMTSSA/98/606 polyprotein |
| | | (AF298808) |
| 344) | dengu36_21mers_1MM.txt | Dengue virus type 1 Djibouti strain D1/H/IMTSSA/98/606 polyprotein |
| | | (AF298808) |
| 345) | dengu36_21mers_2MM.txt | Dengue virus type 1 Djibouti strain D1/H/IMTSSA/98/606 polyprotein |
| | | (AF298808) |
| 346) | dengu36_22mers_0MM.txt | Dengue virus type 1 Djibouti strain D1/H/IMTSSA/98/606 polyprotein |
| | | (AF298808) |
| 347) | dengu36_22mers_1MM.txt | Dengue virus type 1 Djibouti strain D1/H/IMTSSA/98/606 polyprotein |
| | | (AF298808) |
| 348) | dengu36_22mers_2MM.txt | Dengue virus type 1 Djibouti strain D1/H/IMTSSA/98/606 polyprotein |
| | | (AF298808) |
| 349) | dengu37_19mers_0MM.txt | Dengue virus type 1 from Cambodia (AF309641) |
| 350) | dengu37_19mers_1MM.txt | Dengue virus type 1 from Cambodia (AF309641) |
| 351) | dengu37_19mers_2MM.txt | Dengue virus type 1 from Cambodia (AF309641) |
| 352) | dengu37_20mers_0MM.txt | Dengue virus type 1 from Cambodia (AF309641) |
| 353) | dengu37_20mers_1MM.txt | Dengue virus type 1 from Cambodia (AF309641) |
| 354) | dengu37_20mers_2MM.txt | Dengue virus type 1 from Cambodia (AF309641) |
| 355) | dengu37_21mers_0MM.txt | Dengue virus type 1 from Cambodia (AF309641) |
| 356) | dengu37_21mers_1MM.txt | Dengue virus type 1 from Cambodia (AF309641) |
| 357) | dengu37_21mers_2MM.txt | Dengue virus type 1 from Cambodia (AF309641) |
| 358) | dengu37_22mers_0MM.txt | Dengue virus type 1 from Cambodia (AF309641) |
| 359) | dengu37_22mers_1MM.txt | Dengue virus type 1 from Cambodia (AF309641) |
| 360) | dengu37_22mers_2MM.txt | Dengue virus type 1 from Cambodia (AF309641) |
| 361) | dengu38_19mers_0MM.txt | Dengue virus type 2 strain TSV01 (AY037116) |
| 362) | dengu38_19mers_1MM.txt | Dengue virus type 2 strain TSV01 (AY037116) |
| 363) | dengu38_19mers_2MM.txt | Dengue virus type 2 strain TSV01 (AY037116) |
| 364) | dengu38_20mers_0MM.txt | Dengue virus type 2 strain TSV01 (AY037116) |
| 365) | dengu38_20mers_1MM.txt | Dengue virus type 2 strain TSV01 (AY037116) |
| 366) | dengu38_20mers_2MM.txt | Dengue virus type 2 strain TSV01 (AY037116) |
| 367) | dengu38_21mers_0MM.txt | Dengue virus type 2 strain TSV01 (AY037116) |
| 368) | dengu38_21mers_1MM.txt | Dengue virus type 2 strain TSV01 (AY037116) |
| 369) | dengu38_21mers_2MM.txt | Dengue virus type 2 strain TSV01 (AY037116) |
| 370) | dengu38_22mers_0MM.txt | Dengue virus type 2 strain TSV01 (AY037116) |
| 371) | dengu38_22mers_1MM.txt | Dengue virus type 2 strain TSV01 (AY037116) |
| 372) | dengu38_22mers_2MM.txt | Dengue virus type 2 strain TSV01 (AY037116) |
| 373) | dengu39_19mers_0MM.txt | Dengue virus type 1 genomic RNA (AB074760) |
| 374) | dengu39_19mers_1MM.txt | Dengue virus type 1 genomic RNA (AB074760) |
| 375) | dengu39_19mers_2MM.txt | Dengue virus type 1 genomic RNA (AB074760) |
| 376) | dengu39_20mers_0MM.txt | Dengue virus type 1 genomic RNA (AB074760) |
| 377) | dengu39_20mers_1MM.txt | Dengue virus type 1 genomic RNA (AB074760) |
| 378) | dengu39_20mers_2MM.txt | Dengue virus type 1 genomic RNA (AB074760) |
| 379) | dengu39_21mers_0MM.txt | Dengue virus type 1 genomic RNA (AB074760) |
| 380) | dengu39_21mers_1MM.txt | Dengue virus type 1 genomic RNA (AB074760) |
| 381) | dengu39_21mers_2MM.txt | Dengue virus type 1 genomic RNA (AB074760) |
| 382) | dengu39_22mers_0MM.txt | Dengue virus type 1 genomic RNA (AB074760) |
| 383) | dengu39_22mers_1MM.txt | Dengue virus type 1 genomic RNA (AB074760) |
| 384) | dengu39_22mers_2MM.txt | Dengue virus type 1 genomic RNA (AB074760) |
| 385) | dengu4_19mers_0MM.txt | Dengue virus type 1 clone 45AZ5 (DVU88536) |
| 386) | dengu4_19mers_1MM.txt | Dengue virus type 1 clone 45AZ5 (DVU88536) |
| 387) | dengu4_19mers_2MM.txt | Dengue virus type 1 clone 45AZ5 (DVU88536) |
| 388) | dengu4_20mers_0MM.txt | Dengue virus type 1 clone 45AZ5 (DVU88536) |
| 389) | dengu4_20mers_1MM.txt | Dengue virus type 1 clone 45AZ5 (DVU88536) |
| 390) | dengu4_20mers_2MM.txt | Dengue virus type 1 clone 45AZ5 (DVU88536) |
| 391) | dengu4_21mers_0MM.txt | Dengue virus type 1 clone 45AZ5 (DVU88536) |
| 392) | dengu4_21mers_1MM.txt | Dengue virus type 1 clone 45AZ5 (DVU88536) |
| 393) | dengu4_21mers_2MM.txt | Dengue virus type 1 clone 45AZ5 (DVU88536) |
| 394) | dengu4_22mers_0MM.txt | Dengue virus type 1 clone 45AZ5 (DVU88536) |
| 395) | dengu4_22mers_1MM.txt | Dengue virus type 1 clone 45AZ5 (DVU88536) |
| 396) | dengu4_22mers_2MM.txt | Dengue virus type 1 clone 45AZ5 (DVU88536) |
| 397) | dengu40_19mers_0MM.txt | Dengue virus type 1 genomic RNA (AB074761) |
| 398) | dengu40_19mers_1MM.txt | Dengue virus type 1 genomic RNA (AB074761) |
| 399) | dengu40_19mers_2MM.txt | Dengue virus type 1 genomic RNA (AB074761) |
| 400) | dengu40_20mers_0MM.txt | Dengue virus type 1 genomic RNA (AB074761) |
| 401) | dengu40_20mers_1MM.txt | Dengue virus type 1 genomic RNA (AB074761) |
| 402) | dengu40_20mers_2MM.txt | Dengue virus type 1 genomic RNA (AB074761) |
| 403) | dengu40_21mers_0MM.txt | Dengue virus type 1 genomic RNA (AB074761) |
| 404) | dengu40_21mers_1MM.txt | Dengue virus type 1 genomic RNA (AB074761) |
| 405) | dengu40_21mers_2MM.txt | Dengue virus type 1 genomic RNA (AB074761) |
| 406) | dengu40_22mers_0MM.txt | Dengue virus type 1 genomic RNA (AB074761) |
| 407) | dengu40_22mers_1MM.txt | Dengue virus type 1 genomic RNA (AB074761) |
| 408) | dengu40_22mers_2MM.txt | Dengue virus type 1 genomic RNA (AB074761) |
| 409) | dengu41_19mers_0MM.txt | Dengue Virus Type 2 strain BR64022 (AF489932) |
| 410) | dengu41_19mers_1MM.txt | Dengue Virus Type 2 strain BR64022 (AF489932) |
| 411) | dengu41_19mers_2MM.txt | Dengue Virus Type 2 strain BR64022 (AF489932) |
| 412) | dengu41_20mers_0MM.txt | Dengue Virus Type 2 strain BR64022 (AF489932) |
| 413) | dengu41_20mers_1MM.txt | Dengue Virus Type 2 strain BR64022 (AF489932) |
| 414) | dengu41_20mers_2MM.txt | Dengue Virus Type 2 strain BR64022 (AF489932) |
| 415) | dengu41_21mers_0MM.txt | Dengue Virus Type 2 strain BR64022 (AF489932) |
| 416) | dengu41_21mers_1MM.txt | Dengue Virus Type 2 strain BR64022 (AF489932) |
| 417) | dengu41_21mers_2MM.txt | Dengue Virus Type 2 strain BR64022 (AF489932) |
| 418) | dengu41_22mers_0MM.txt | Dengue Virus Type 2 strain BR64022 (AF489932) |
| 419) | dengu41_22mers_1MM.txt | Dengue Virus Type 2 strain BR64022 (AF489932) |
| 420) | dengu41_22mers_2MM.txt | Dengue Virus Type 2 strain BR64022 (AF489932) |
| 421) | dengu42_19mers_0MM.txt | Dengue virus type 1 recombinant clone rDEN1 (AY145121) |
| 422) | dengu42_19mers_1MM.txt | Dengue virus type 1 recombinant clone rDEN1 (AY145121) |
| 423) | dengu42_19mers_2MM.txt | Dengue virus type 1 recombinant clone rDEN1 (AY145121) |
| 424) | dengu42_20mers_0MM.txt | Dengue virus type 1 recombinant clone rDEN1 (AY145121) |
| 425) | dengu42_20mers_1MM.txt | Dengue virus type 1 recombinant clone rDEN1 (AY145121) |
| 426) | dengu42_20mers_2MM.txt | Dengue virus type 1 recombinant clone rDEN1 (AY145121) |
| 427) | dengu42_21mers_0MM.txt | Dengue virus type 1 recombinant clone rDEN1 (AY145121) |
| 428) | dengu42_21mers_1MM.txt | Dengue virus type 1 recombinant clone rDEN1 (AY145121) |
| 429) | dengu42_21mers_2MM.txt | Dengue virus type 1 recombinant clone rDEN1 (AY145121) |
| 430) | dengu42_22mers_0MM.txt | Dengue virus type 1 recombinant clone rDEN1 (AY145121) |
| 431) | dengu42_22mers_1MM.txt | Dengue virus type 1 recombinant clone rDEN1 (AY145121) |
| 432) | dengu42_22mers_2MM.txt | Dengue virus type 1 recombinant clone rDEN1 (AY145121) |
| 433) | dengu43_19mers_0MM.txt | Dengue virus type 1 recombinant clone rDEN1mutF (AY145122) |
| 434) | dengu43_19mers_1MM.txt | Dengue virus type 1 recombinant clone rDEN1mutF (AY145122) |
| 435) | dengu43_19mers_2MM.txt | Dengue virus type 1 recombinant clone rDEN1mutF (AY145122) |
| 436) | dengu43_20mers_0MM.txt | Dengue virus type 1 recombinant clone rDEN1mutF (AY145122) |
| 437) | dengu43_20mers_1MM.txt | Dengue virus type 1 recombinant clone rDEN1mutF (AY145122) |
| 438) | dengu43_20mers_2MM.txt | Dengue virus type 1 recombinant clone rDEN1mutF (AY145122) |
| 439) | dengu43_21mers_0MM.txt | Dengue virus type 1 recombinant clone rDEN1mutF (AY145122) |
| 440) | dengu43_21mers_1MM.txt | Dengue virus type 1 recombinant clone rDEN1mutF (AY145122) |
| 441) | dengu43_21mers_2MM.txt | Dengue virus type 1 recombinant clone rDEN1mutF (AY145122) |
| 442) | dengu43_22mers_0MM.txt | Dengue virus type 1 recombinant clone rDEN1mutF (AY145122) |
| 443) | dengu43_22mers_1MM.txt | Dengue virus type 1 recombinant clone rDEN1mutF (AY145122) |
| 444) | dengu43_22mers_2MM.txt | Dengue virus type 1 recombinant clone rDEN1mutF (AY145122) |
| 445) | dengu44_19mers_0MM.txt | Dengue virus type 1 recombinant clone rDEN1delta30 (AY145123) |
| 446) | dengu44_19mers_1MM.txt | Dengue virus type 1 recombinant clone rDEN1delta30 (AY145123) |
| 447) | dengu44_19mers_2MM.txt | Dengue virus type 1 recombinant clone rDEN1delta30 (AY145123) |
| 448) | dengu44_20mers_0MM.txt | Dengue virus type 1 recombinant clone rDEN1delta30 (AY145123) |
| 449) | dengu44_20mers_1MM.txt | Dengue virus type 1 recombinant clone rDEN1delta30 (AY145123) |
| 450) | dengu44_20mers_2MM.txt | Dengue virus type 1 recombinant clone rDEN1delta30 (AY145123) |
| 451) | dengu44_21mers_0MM.txt | Dengue virus type 1 recombinant clone rDEN1delta30 (AY145123) |
| 452) | dengu44_21mers_1MM.txt | Dengue virus type 1 recombinant clone rDEN1delta30 (AY145123) |
| 453) | dengu44_21mers_2MM.txt | Dengue virus type 1 recombinant clone rDEN1delta30 (AY145123) |
| 454) | dengu44_22mers_0MM.txt | Dengue virus type 1 recombinant clone rDEN1delta30 (AY145123) |
| 455) | dengu44_22mers_1MM.txt | Dengue virus type 1 recombinant clone rDEN1delta30 (AY145123) |
| 456) | dengu44_22mers_2MM.txt | Dengue virus type 1 recombinant clone rDEN1delta30 (AY145123) |
| 457) | dengu45_19mers_0MM.txt | Dengue virus type 1 strain Den1BR/90 (AF226685) |
| 458) | dengu45_19mers_1MM.txt | Dengue virus type 1 strain Den1BR/90 (AF226685) |
| 459) | dengu45_19mers_2MM.txt | Dengue virus type 1 strain Den1BR/90 (AF226685) |
| 460) | dengu45_20mers_0MM.txt | Dengue virus type 1 strain Den1BR/90 (AF226685) |
| 461) | dengu45_20mers_1MM.txt | Dengue virus type 1 strain Den1BR/90 (AF226685) |
| 462) | dengu45_20mers_2MM.txt | Dengue virus type 1 strain Den1BR/90 (AF226685) |
| 463) | dengu45_21mers_0MM.txt | Dengue virus type 1 strain Den1BR/90 (AF226685) |
| 464) | dengu45_21mers_1MM.txt | Dengue virus type 1 strain Den1BR/90 (AF226685) |
| 465) | dengu45_21mers_2MM.txt | Dengue virus type 1 strain Den1BR/90 (AF226685) |
| 466) | dengu45_22mers_0MM.txt | Dengue virus type 1 strain Den1BR/90 (AF226685) |
| 467) | dengu45_22mers_1MM.txt | Dengue virus type 1 strain Den1BR/90 (AF226685) |
| 468) | dengu45_22mers_2MM.txt | Dengue virus type 1 strain Den1BR/90 (AF226685) |
| 469) | dengu46_19mers_0MM.txt | Dengue virus type 1 strain BR/97-111 (AF311956) |
| 470) | dengu46_19mers_1MM.txt | Dengue virus type 1 strain BR/97-111 (AF311956) |
| 471) | dengu46_19mers_2MM.txt | Dengue virus type 1 strain BR/97-111 (AF311956) |
| 472) | dengu46_20mers_0MM.txt | Dengue virus type 1 strain BR/97-111 (AF311956) |
| 473) | dengu46_20mers_1MM.txt | Dengue virus type 1 strain BR/97-111 (AF311956) |
| 474) | dengu46_20mers_2MM.txt | Dengue virus type 1 strain BR/97-111 (AF311956) |
| 475) | dengu46_21mers_0MM.txt | Dengue virus type 1 strain BR/97-111 (AF311956) |
| 476) | dengu46_21mers_1MM.txt | Dengue virus type 1 strain BR/97-111 (AF311956) |
| 477) | dengu46_21mers_2MM.txt | Dengue virus type 1 strain BR/97-111 (AF311956) |
| 478) | dengu46_22mers_0MM.txt | Dengue virus type 1 strain BR/97-111 (AF311956) |
| 479) | dengu46_22mers_1MM.txt | Dengue virus type 1 strain BR/97-111 (AF311956) |
| 480) | dengu46_22mers_2MM.txt | Dengue virus type 1 strain BR/97-111 (AF311956) |
| 481) | dengu47_19mers_0MM.txt | Dengue virus type 1 strain BR/97-409 (AF311957) |
| 482) | dengu47_19mers_1MM.txt | Dengue virus type 1 strain BR/97-409 (AF311957) |
| 483) | dengu47_19mers_2MM.txt | Dengue virus type 1 strain BR/97-409 (AF311957) |
| 484) | dengu47_20mers_0MM.txt | Dengue virus type 1 strain BR/97-409 (AF311957) |
| 485) | dengu47_20mers_1MM.txt | Dengue virus type 1 strain BR/97-409 (AF311957) |
| 486) | dengu47_20mers_2MM.txt | Dengue virus type 1 strain BR/97-409 (AF311957) |
| 487) | dengu47_21mers_0MM.txt | Dengue virus type 1 strain BR/97-409 (AF311957) |
| 488) | dengu47_21mers_1MM.txt | Dengue virus type 1 strain BR/97-409 (AF311957) |
| 489) | dengu47_21mers_2MM.txt | Dengue virus type 1 strain BR/97-409 (AF311957) |
| 490) | dengu47_22mers_0MM.txt | Dengue virus type 1 strain BR/97-409 (AF311957) |
| 491) | dengu47_22mers_1MM.txt | Dengue virus type 1 strain BR/97-409 (AF311957) |
| 492) | dengu47_22mers_2MM.txt | Dengue virus type 1 strain BR/97-409 (AF311957) |
| 493) | dengu48_19mers_0MM.txt | Dengue virus type 1 strain BR/97-233 (AF311958) |
| 494) | dengu48_19mers_1MM.txt | Dengue virus type 1 strain BR/97-233 (AF311958) |
| 495) | dengu48_19mers_2MM.txt | Dengue virus type 1 strain BR/97-233 (AF311958) |
| 496) | dengu48_20mers_0MM.txt | Dengue virus type 1 strain BR/97-233 (AF311958) |
| 497) | dengu48_20mers_1MM.txt | Dengue virus type 1 strain BR/97-233 (AF311958) |
| 498) | dengu48_20mers_2MM.txt | Dengue virus type 1 strain BR/97-233 (AF311958) |
| 499) | dengu48_21mers_0MM.txt | Dengue virus type 1 strain BR/97-233 (AF311958) |
| 500) | dengu48_21mers_1MM.txt | Dengue virus type 1 strain BR/97-233 (AF311958) |
| 501) | dengu48_21mers_2MM.txt | Dengue virus type 1 strain BR/97-233 (AF311958) |
| 502) | dengu48_22mers_0MM.txt | Dengue virus type 1 strain BR/97-233 (AF311958) |
| 503) | dengu48_22mers_1MM.txt | Dengue virus type 1 strain BR/97-233 (AF311958) |
| 504) | dengu48_22mers_2MM.txt | Dengue virus type 1 strain BR/97-233 (AF311958) |
| 505) | dengu49_19mers_0MM.txt | Dengue virus type 1 strain 280par00 polyprotein gene (AF514878) |
| 506) | dengu49_19mers_1MM.txt | Dengue virus type 1 strain 280par00 polyprotein gene (AF514878) |
| 507) | dengu49_19mers_2MM.txt | Dengue virus type 1 strain 280par00 polyprotein gene (AF514878) |
| 508) | dengu49_20mers_0MM.txt | Dengue virus type 1 strain 280par00 polyprotein gene (AF514878) |
| 509) | dengu49_20mers_1MM.txt | Dengue virus type 1 strain 280par00 polyprotein gene (AF514878) |
| 510) | dengu49_20mers_2MM.txt | Dengue virus type 1 strain 280par00 polyprotein gene (AF514878) |
| 511) | dengu49_21mers_0MM.txt | Dengue virus type 1 strain 280par00 polyprotein gene (AF514878) |
| 512) | dengu49_21mers_1MM.txt | Dengue virus type 1 strain 280par00 polyprotein gene (AF514878) |
| 513) | dengu49_21mers_2MM.txt | Dengue virus type 1 strain 280par00 polyprotein gene (AF514878) |
| 514) | dengu49_22mers_0MM.txt | Dengue virus type 1 strain 280par00 polyprotein gene (AF514878) |
| 515) | dengu49_22mers_1MM.txt | Dengue virus type 1 strain 280par00 polyprotein gene (AF514878) |
| 516) | dengu49_22mers_2MM.txt | Dengue virus type 1 strain 280par00 polyprotein gene (AF514878) |
| 517) | dengu5_19mers_0MM.txt | Dengue virus type 2 (strain 16681) polyprotein mRNA (U87411) |
| 518) | dengu5_19mers_1MM.txt | Dengue virus type 2 (strain 16681) polyprotein mRNA (U87411) |
| 519) | dengu5_19mers_2MM.txt | Dengue virus type 2 (strain 16681) polyprotein mRNA (U87411) |
| 520) | dengu5_20mers_0MM.txt | Dengue virus type 2 (strain 16681) polyprotein mRNA (U87411) |
| 521) | dengu5_20mers_1MM.txt | Dengue virus type 2 (strain 16681) polyprotein mRNA (U87411) |
| 522) | dengu5_20mers_2MM.txt | Dengue virus type 2 (strain 16681) polyprotein mRNA (U87411) |
| 523) | dengu5_21mers_0MM.txt | Dengue virus type 2 (strain 16681) polyprotein mRNA (U87411) |
| 524) | dengu5_21mers_1MM.txt | Dengue virus type 2 (strain 16681) polyprotein mRNA (U87411) |
| 525) | dengu5_21mers_2MM.txt | Dengue virus type 2 (strain 16681) polyprotein mRNA (U87411) |
| 526) | dengu5_22mers_0MM.txt | Dengue virus type 2 (strain 16681) polyprotein mRNA (U87411) |
| 527) | dengu5_22mers_1MM.txt | Dengue virus type 2 (strain 16681) polyprotein mRNA (U87411) |
| 528) | dengu5_22mers_2MM.txt | Dengue virus type 2 (strain 16681) polyprotein mRNA (U87411) |
| 529) | dengu50_19mers_0MM.txt | Dengue virus type 1 strain 297arg00 envelope glycoprotein gene |
| | | (AF514889) |
| 530) | dengu50_19mers_1MM.txt | Dengue virus type 1 strain 297arg00 envelope glycoprotein gene |
| | | (AF514889) |
| 531) | dengu50_19mers_2MM.txt | Dengue virus type 1 strain 297arg00 envelope glycoprotein gene |
| | | (AF514889) |
| 532) | dengu50_20mers_0MM.txt | Dengue virus type 1 strain 297arg00 envelope glycoprotein gene |
| | | (AF514889) |
| 533) | dengu50_20mers_1MM.txt | Dengue virus type 1 strain 297arg00 envelope glycoprotein gene |
| | | (AF514889) |
| 534) | dengu50_20mers_2MM.txt | Dengue virus type 1 strain 297arg00 envelope glycoprotein gene |
| | | (AF514889) |
| 535) | dengu50_21mers_0MM.txt | Dengue virus type 1 strain 297arg00 envelope glycoprotein gene |
| | | (AF514889) |
| 536) | dengu50_21mers_1MM.txt | Dengue virus type 1 strain 297arg00 envelope glycoprotein gene |
| | | (AF514889) |
| 537) | dengu50_21mers_2MM.txt | Dengue virus type 1 strain 297arg00 envelope glycoprotein gene |
| | | (AF514889) |
| 538) | dengu50_22mers_0MM.txt | Dengue virus type 1 strain 297arg00 envelope glycoprotein gene |
| | | (AF514889) |
| 539) | dengu50_22mers_1MM.txt | Dengue virus type 1 strain 297arg00 envelope glycoprotein gene |
| | | (AF514889) |
| 540) | dengu50_22mers_2MM.txt | Dengue virus type 1 strain 297arg00 envelope glycoprotein gene |
| | | (AF514889) |
| 541) | dengu51_19mers_0MM.txt | Dengue virus type 1 isolate BR/01-MR (AF513110) |
| 542) | dengu51_19mers_1MM.txt | Dengue virus type 1 isolate BR/01-MR (AF513110) |
| 543) | dengu51_19mers_2MM.txt | Dengue virus type 1 isolate BR/01-MR (AF513110) |
| 544) | dengu51_20mers_0MM.txt | Dengue virus type 1 isolate BR/01-MR (AF513110) |
| 545) | dengu51_20mers_1MM.txt | Dengue virus type 1 isolate BR/01-MR (AF513110) |
| 546) | dengu51_20mers_2MM.txt | Dengue virus type 1 isolate BR/01-MR (AF513110) |
| 547) | dengu51_21mers_0MM.txt | Dengue virus type 1 isolate BR/01-MR (AF513110) |
| 548) | dengu51_21mers_1MM.txt | Dengue virus type 1 isolate BR/01-MR (AF513110) |
| 549) | dengu51_21mers_2MM.txt | Dengue virus type 1 isolate BR/01-MR (AF513110) |
| 550) | dengu51_22mers_0MM.txt | Dengue virus type 1 isolate BR/01-MR (AF513110) |
| 551) | dengu51_22mers_1MM.txt | Dengue virus type 1 isolate BR/01-MR (AF513110) |
| 552) | dengu51_22mers_2MM.txt | Dengue virus type 1 isolate BR/01-MR (AF513110) |
| 553) | dengu52_19mers_0MM.txt | Dengue virus type 1 strain 301arg00 polyprotein gene (AF514876) |
| 554) | dengu52_19mers_1MM.txt | Dengue virus type 1 strain 301arg00 polyprotein gene (AF514876) |
| 555) | dengu52_19mers_2MM.txt | Dengue virus type 1 strain 301arg00 polyprotein gene (AF514876) |
| 556) | dengu52_20mers_0MM.txt | Dengue virus type 1 strain 301arg00 polyprotein gene (AF514876) |
| 557) | dengu52_20mers_1MM.txt | Dengue virus type 1 strain 301arg00 polyprotein gene (AF514876) |
| 558) | dengu52_20mers_2MM.txt | Dengue virus type 1 strain 301arg00 polyprotein gene (AF514876) |
| 559) | dengu52_21mers_0MM.txt | Dengue virus type 1 strain 301arg00 polyprotein gene (AF514876) |
| 560) | dengu52_21mers_1MM.txt | Dengue virus type 1 strain 301arg00 polyprotein gene (AF514876) |
| 561) | dengu52_21mers_2MM.txt | Dengue virus type 1 strain 301arg00 polyprotein gene (AF514876) |
| 562) | dengu52_22mers_0MM.txt | Dengue virus type 1 strain 301arg00 polyprotein gene (AF514876) |
| 563) | dengu52_22mers_1MM.txt | Dengue virus type 1 strain 301arg00 polyprotein gene (AF514876) |
| 564) | dengu52_22mers_2MM.txt | Dengue virus type 1 strain 301arg00 polyprotein gene (AF514876) |
| 565) | dengu53_19mers_0MM.txt | Dengue virus type 1 strain 295arg00 polyprotein gene (AF514885) |
| 566) | dengu53_19mers_1MM.txt | Dengue virus type 1 strain 295arg00 polyprotein gene (AF514885) |
| 567) | dengu53_19mers_2MM.txt | Dengue virus type 1 strain 295arg00 polyprotein gene (AF514885) |
| 568) | dengu53_20mers_0MM.txt | Dengue virus type 1 strain 295arg00 polyprotein gene (AF514885) |
| 569) | dengu53_20mers_1MM.txt | Dengue virus type 1 strain 295arg00 polyprotein gene (AF514885) |
| 570) | dengu53_20mers_2MM.txt | Dengue virus type 1 strain 295arg00 polyprotein gene (AF514885) |
| 571) | dengu53_21mers_0MM.txt | Dengue virus type 1 strain 295arg00 polyprotein gene (AF514885) |
| 572) | dengu53_21mers_1MM.txt | Dengue virus type 1 strain 295arg00 polyprotein gene (AF514885) |
| 573) | dengu53_21mers_2MM.txt | Dengue virus type 1 strain 295arg00 polyprotein gene (AF514885) |
| 574) | dengu53_22mers_0MM.txt | Dengue virus type 1 strain 295arg00 polyprotein gene (AF514885) |
| 575) | dengu53_22mers_1MM.txt | Dengue virus type 1 strain 295arg00 polyprotein gene (AF514885) |
| 576) | dengu53_22mers_2MM.txt | Dengue virus type 1 strain 295arg00 polyprotein gene (AF514885) |
| 577) | dengu54_19mers_0MM.txt | Dengue virus type 1 strain 259par00 polyprotein gene (AF514883) |
| 578) | dengu54_19mers_1MM.txt | Dengue virus type 1 strain 259par00 polyprotein gene (AF514883) |
| 579) | dengu54_19mers_2MM.txt | Dengue virus type 1 strain 259par00 polyprotein gene (AF514883) |
| 580) | dengu54_20mers_0MM.txt | Dengue virus type 1 strain 259par00 polyprotein gene (AF514883) |
| 581) | dengu54_20mers_1MM.txt | Dengue virus type 1 strain 259par00 polyprotein gene (AF514883) |
| 582) | dengu54_20mers_2MM.txt | Dengue virus type 1 strain 259par00 polyprotein gene (AF514883) |
| 583) | dengu54_21mers_0MM.txt | Dengue virus type 1 strain 259par00 polyprotein gene (AF514883) |
| 584) | dengu54_21mers_1MM.txt | Dengue virus type 1 strain 259par00 polyprotein gene (AF514883) |
| 585) | dengu54_21mers_2MM.txt | Dengue virus type 1 strain 259par00 polyprotein gene (AF514883) |
| 586) | dengu54_22mers_0MM.txt | Dengue virus type 1 strain 259par00 polyprotein gene (AF514883) |
| 587) | dengu54_22mers_1MM.txt | Dengue virus type 1 strain 259par00 polyprotein gene (AF514883) |
| 588) | dengu54_22mers_2MM.txt | Dengue virus type 1 strain 259par00 polyprotein gene (AF514883) |
| 589) | dengu55_19mers_0MM.txt | Dengue virus type 1 strain 293arg00 polyprotein gene (AY206457) |
| 590) | dengu55_19mers_1MM.txt | Dengue virus type 1 strain 293arg00 polyprotein gene (AY206457) |
| 591) | dengu55_19mers_2MM.txt | Dengue virus type 1 strain 293arg00 polyprotein gene (AY206457) |
| 592) | dengu55_20mers_0MM.txt | Dengue virus type 1 strain 293arg00 polyprotein gene (AY206457) |
| 593) | dengu55_20mers_1MM.txt | Dengue virus type 1 strain 293arg00 polyprotein gene (AY206457) |
| 594) | dengu55_20mers_2MM.txt | Dengue virus type 1 strain 293arg00 polyprotein gene (AY206457) |
| 595) | dengu55_21mers_0MM.txt | Dengue virus type 1 strain 293arg00 polyprotein gene (AY206457) |
| 596) | dengu55_21mers_1MM.txt | Dengue virus type 1 strain 293arg00 polyprotein gene (AY206457) |
| 597) | dengu55_21mers_2MM.txt | Dengue virus type 1 strain 293arg00 polyprotein gene (AY206457) |
| 598) | dengu55_22mers_0MM.txt | Dengue virus type 1 strain 293arg00 polyprotein gene (AY206457) |
| 599) | dengu55_22mers_1MM.txt | Dengue virus type 1 strain 293arg00 polyprotein gene (AY206457) |
| 600) | dengu55_22mers_2MM.txt | Dengue virus type 1 strain 293arg00 polyprotein gene (AY206457) |
| 601) | dengu56_19mers_0MM.txt | Dengue virus type 2 complete genome. (DEN2CGA) |
| 602) | dengu56_19mers_1MM.txt | Dengue virus type 2 complete genome. (DEN2CGA) |
| 603) | dengu56_19mers_2MM.txt | Dengue virus type 2 complete genome. (DEN2CGA) |
| 604) | dengu56_20mers_0MM.txt | Dengue virus type 2 complete genome. (DEN2CGA) |
| 605) | dengu56_20mers_1MM.txt | Dengue virus type 2 complete genome. (DEN2CGA) |
| 606) | dengu56_20mers_2MM.txt | Dengue virus type 2 complete genome. (DEN2CGA) |
| 607) | dengu56_21mers_0MM.txt | Dengue virus type 2 complete genome. (DEN2CGA) |
| 608) | dengu56_21mers_1MM.txt | Dengue virus type 2 complete genome. (DEN2CGA) |
| 609) | dengu56_21mers_2MM.txt | Dengue virus type 2 complete genome. (DEN2CGA) |
| 610) | dengu56_22mers_0MM.txt | Dengue virus type 2 complete genome. (DEN2CGA) |
| 611) | dengu56_22mers_1MM.txt | Dengue virus type 2 complete genome. (DEN2CGA) |
| 612) | dengu56_22mers_2MM.txt | Dengue virus type 2 complete genome. (DEN2CGA) |
| 613) | dengu57_19mers_0MM.txt | Dengue virus type 2 Jamaica/N.1409 (DEN2JAMCG) |
| 614) | dengu57_19mers_1MM.txt | Dengue virus type 2 Jamaica/N.1409 (DEN2JAMCG) |
| 615) | dengu57_19mers_2MM.txt | Dengue virus type 2 Jamaica/N.1409 (DEN2JAMCG) |
| 616) | dengu57_20mers_0MM.txt | Dengue virus type 2 Jamaica/N.1409 (DEN2JAMCG) |
| 617) | dengu57_20mers_1MM.txt | Dengue virus type 2 Jamaica/N.1409 (DEN2JAMCG) |
| 618) | dengu57_20mers_2MM.txt | Dengue virus type 2 Jamaica/N.1409 (DEN2JAMCG) |
| 619) | dengu57_21mers_0MM.txt | Dengue virus type 2 Jamaica/N.1409 (DEN2JAMCG) |
| 620) | dengu57_21mers_1MM.txt | Dengue virus type 2 Jamaica/N.1409 (DEN2JAMCG) |
| 621) | dengu57_21mers_2MM.txt | Dengue virus type 2 Jamaica/N.1409 (DEN2JAMCG) |
| 622) | dengu57_22mers_0MM.txt | Dengue virus type 2 Jamaica/N.1409 (DEN2JAMCG) |
| 623) | dengu57_22mers_1MM.txt | Dengue virus type 2 Jamaica/N.1409 (DEN2JAMCG) |
| 624) | dengu57_22mers_2MM.txt | Dengue virus type 2 Jamaica/N.1409 (DEN2JAMCG) |
| 625) | dengu58_19mers_0MM.txt | Dengue virus type 1 complete genome. (DENT1SEQ) |
| 626) | dengu58_19mers_1MM.txt | Dengue virus type 1 complete genome. (DENT1SEQ) |
| 627) | dengu58_19mers_2MM.txt | Dengue virus type 1 complete genome. (DENT1SEQ) |
| 628) | dengu58_20mers_0MM.txt | Dengue virus type 1 complete genome. (DENT1SEQ) |
| 629) | dengu58_20mers_1MM.txt | Dengue virus type 1 complete genome. (DENT1SEQ) |
| 630) | dengu58_20mers_2MM.txt | Dengue virus type 1 complete genome. (DENT1SEQ) |
| 631) | dengu58_21mers_0MM.txt | Dengue virus type 1 complete genome. (DENT1SEQ) |
| 632) | dengu58_21mers_1MM.txt | Dengue virus type 1 complete genome. (DENT1SEQ) |
| 633) | dengu58_21mers_2MM.txt | Dengue virus type 1 complete genome. (DENT1SEQ) |
| 634) | dengu58_22mers_0MM.txt | Dengue virus type 1 complete genome. (DENT1SEQ) |
| 635) | dengu58_22mers_1MM.txt | Dengue virus type 1 complete genome. (DENT1SEQ) |
| 636) | dengu58_22mers_2MM.txt | Dengue virus type 1 complete genome. (DENT1SEQ) |
| 637) | dengu59_19mers_0MM.txt | Dengue virus type 1 clone WestPac (DVU88535) |
| 638) | dengu59_19mers_1MM.txt | Dengue virus type 1 clone WestPac (DVU88535) |
| 639) | dengu59_19mers_2MM.txt | Dengue virus type 1 clone WestPac (DVU88535) |
| 640) | dengu59_20mers_0MM.txt | Dengue virus type 1 clone WestPac (DVU88535) |
| 641) | dengu59_20mers_1MM.txt | Dengue virus type 1 clone WestPac (DVU88535) |
| 642) | dengu59_20mers_2MM.txt | Dengue virus type 1 clone WestPac (DVU88535) |
| 643) | dengu59_21mers_0MM.txt | Dengue virus type 1 clone WestPac (DVU88535) |
| 644) | dengu59_21mers_1MM.txt | Dengue virus type 1 clone WestPac (DVU88535) |
| 645) | dengu59_21mers_2MM.txt | Dengue virus type 1 clone WestPac (DVU88535) |
| 646) | dengu59_22mers_0MM.txt | Dengue virus type 1 clone WestPac (DVU88535) |
| 647) | dengu59_22mers_1MM.txt | Dengue virus type 1 clone WestPac (DVU88535) |
| 648) | dengu59_22mers_2MM.txt | Dengue virus type 1 clone WestPac (DVU88535) |
| 649) | dengu6_19mers_0MM.txt | Dengue virus type 2 polyprotein mRNA (U87412) |
| 650) | dengu6_19mers_1MM.txt | Dengue virus type 2 polyprotein mRNA (U87412) |
| 651) | dengu6_19mers_2MM.txt | Dengue virus type 2 polyprotein mRNA (U87412) |
| 652) | dengu6_20mers_0MM.txt | Dengue virus type 2 polyprotein mRNA (U87412) |
| 653) | dengu6_20mers_1MM.txt | Dengue virus type 2 polyprotein mRNA (U87412) |
| 654) | dengu6_20mers_2MM.txt | Dengue virus type 2 polyprotein mRNA (U87412) |
| 655) | dengu6_21mers_0MM.txt | Dengue virus type 2 polyprotein mRNA (U87412) |
| 656) | dengu6_21mers_1MM.txt | Dengue virus type 2 polyprotein mRNA (U87412) |
| 657) | dengu6_21mers_2MM.txt | Dengue virus type 2 polyprotein mRNA (U87412) |
| 658) | dengu6_22mers_0MM.txt | Dengue virus type 2 polyprotein mRNA (U87412) |
| 659) | dengu6_22mers_1MM.txt | Dengue virus type 2 polyprotein mRNA (U87412) |
| 660) | dengu6_22mers_2MM.txt | Dengue virus type 2 polyprotein mRNA (U87412) |
| 661) | dengu60_19mers_0MM.txt | Dengue virus type 1 clone 45AZ5-PDK27 (DVU88537) |
| 662) | dengu60_19mers_1MM.txt | Dengue virus type 1 clone 45AZ5-PDK27 (DVU88537) |
| 663) | dengu60_19mers_2MM.txt | Dengue virus type 1 clone 45AZ5-PDK27 (DVU88537) |
| 664) | dengu60_20mers_0MM.txt | Dengue virus type 1 clone 45AZ5-PDK27 (DVU88537) |
| 665) | dengu60_20mers_1MM.txt | Dengue virus type 1 clone 45AZ5-PDK27 (DVU88537) |
| 666) | dengu60_20mers_2MM.txt | Dengue virus type 1 clone 45AZ5-PDK27 (DVU88537) |
| 667) | dengu60_21mers_0MM.txt | Dengue virus type 1 clone 45AZ5-PDK27 (DVU88537) |
| 668) | dengu60_21mers_1MM.txt | Dengue virus type 1 clone 45AZ5-PDK27 (DVU88537) |
| 669) | dengu60_21mers_2MM.txt | Dengue virus type 1 clone 45AZ5-PDK27 (DVU88537) |
| 670) | dengu60_22mers_0MM.txt | Dengue virus type 1 clone 45AZ5-PDK27 (DVU88537) |
| 671) | dengu60_22mers_1MM.txt | Dengue virus type 1 clone 45AZ5-PDK27 (DVU88537) |
| 672) | dengu60_22mers_2MM.txt | Dengue virus type 1 clone 45AZ5-PDK27 (DVU88537) |
| 673) | dengu61_19mers_0MM.txt | Dengue virus type 2 strain ThNH-7/93 (AF022434) |
| 674) | dengu61_19mers_1MM.txt | Dengue virus type 2 strain ThNH-7/93 (AF022434) |
| 675) | dengu61_19mers_2MM.txt | Dengue virus type 2 strain ThNH-7/93 (AF022434) |
| 676) | dengu61_20mers_0MM.txt | Dengue virus type 2 strain ThNH-7/93 (AF022434) |
| 677) | dengu61_20mers_1MM.txt | Dengue virus type 2 strain ThNH-7/93 (AF022434) |
| 678) | dengu61_20mers_2MM.txt | Dengue virus type 2 strain ThNH-7/93 (AF022434) |
| 679) | dengu61_21mers_0MM.txt | Dengue virus type 2 strain ThNH-7/93 (AF022434) |
| 680) | dengu61_21mers_1MM.txt | Dengue virus type 2 strain ThNH-7/93 (AF022434) |
| 681) | dengu61_21mers_2MM.txt | Dengue virus type 2 strain ThNH-7/93 (AF022434) |
| 682) | dengu61_22mers_0MM.txt | Dengue virus type 2 strain ThNH-7/93 (AF022434) |
| 683) | dengu61_22mers_1MM.txt | Dengue virus type 2 strain ThNH-7/93 (AF022434) |
| 684) | dengu61_22mers_2MM.txt | Dengue virus type 2 strain ThNH-7/93 (AF022434) |
| 685) | dengu62_19mers_0MM.txt | Dengue virus type 2 strain ThNH-28/93 (AF022435) |
| 686) | dengu62_19mers_1MM.txt | Dengue virus type 2 strain ThNH-28/93 (AF022435) |
| 687) | dengu62_19mers_2MM.txt | Dengue virus type 2 strain ThNH-28/93 (AF022435) |
| 688) | dengu62_20mers_0MM.txt | Dengue virus type 2 strain ThNH-28/93 (AF022435) |
| 689) | dengu62_20mers_1MM.txt | Dengue virus type 2 strain ThNH-28/93 (AF022435) |
| 690) | dengu62_20mers_2MM.txt | Dengue virus type 2 strain ThNH-28/93 (AF022435) |
| 691) | dengu62_21mers_0MM.txt | Dengue virus type 2 strain ThNH-28/93 (AF022435) |
| 692) | dengu62_21mers_1MM.txt | Dengue virus type 2 strain ThNH-28/93 (AF022435) |
| 693) | dengu62_21mers_2MM.txt | Dengue virus type 2 strain ThNH-28/93 (AF022435) |
| 694) | dengu62_22mers_0MM.txt | Dengue virus type 2 strain ThNH-28/93 (AF022435) |
| 695) | dengu62_22mers_1MM.txt | Dengue virus type 2 strain ThNH-28/93 (AF022435) |
| 696) | dengu62_22mers_2MM.txt | Dengue virus type 2 strain ThNH-28/93 (AF022435) |
| 697) | dengu63_19mers_0MM.txt | Dengue virus type 2 strain ThNH-52/93 (AF022436) |
| 698) | dengu63_19mers_1MM.txt | Dengue virus type 2 strain ThNH-52/93 (AF022436) |
| 699) | dengu63_19mers_2MM.txt | Dengue virus type 2 strain ThNH-52/93 (AF022436) |
| 700) | dengu63_20mers_0MM.txt | Dengue virus type 2 strain ThNH-52/93 (AF022436) |
| 701) | dengu63_20mers_1MM.txt | Dengue virus type 2 strain ThNH-52/93 (AF022436) |
| 702) | dengu63_20mers_2MM.txt | Dengue virus type 2 strain ThNH-52/93 (AF022436) |
| 703) | dengu63_21mers_0MM.txt | Dengue virus type 2 strain ThNH-52/93 (AF022436) |
| 704) | dengu63_21mers_1MM.txt | Dengue virus type 2 strain ThNH-52/93 (AF022436) |
| 705) | dengu63_21mers_2MM.txt | Dengue virus type 2 strain ThNH-52/93 (AF022436) |
| 706) | dengu63_22mers_0MM.txt | Dengue virus type 2 strain ThNH-52/93 (AF022436) |
| 707) | dengu63_22mers_1MM.txt | Dengue virus type 2 strain ThNH-52/93 (AF022436) |
| 708) | dengu63_22mers_2MM.txt | Dengue virus type 2 strain ThNH-52/93 (AF022436) |
| 709) | dengu64_19mers_0MM.txt | Dengue virus type 2 strain ThNH-p11/93 (AF022437) |
| 710) | dengu64_19mers_1MM.txt | Dengue virus type 2 strain ThNH-p11/93 (AF022437) |
| 711) | dengu64_19mers_2MM.txt | Dengue virus type 2 strain ThNH-p11/93 (AF022437) |
| 712) | dengu64_20mers_0MM.txt | Dengue virus type 2 strain ThNH-p11/93 (AF022437) |
| 713) | dengu64_20mers_1MM.txt | Dengue virus type 2 strain ThNH-p11/93 (AF022437) |
| 714) | dengu64_20mers_2MM.txt | Dengue virus type 2 strain ThNH-p11/93 (AF022437) |
| 715) | dengu64_21mers_0MM.txt | Dengue virus type 2 strain ThNH-p11/93 (AF022437) |
| 716) | dengu64_21mers_1MM.txt | Dengue virus type 2 strain ThNH-p11/93 (AF022437) |
| 717) | dengu64_21mers_2MM.txt | Dengue virus type 2 strain ThNH-p11/93 (AF022437) |
| 718) | dengu64_22mers_0MM.txt | Dengue virus type 2 strain ThNH-p11/93 (AF022437) |
| 719) | dengu64_22mers_1MM.txt | Dengue virus type 2 strain ThNH-p11/93 (AF022437) |
| 720) | dengu64_22mers_2MM.txt | Dengue virus type 2 strain ThNH-p11/93 (AF022437) |
| 721) | dengu65_19mers_0MM.txt | Dengue virus type 2 strain ThNH-p12/93 (AF022438) |
| 722) | dengu65_19mers_1MM.txt | Dengue virus type 2 strain ThNH-p12/93 (AF022438) |
| 723) | dengu65_19mers_2MM.txt | Dengue virus type 2 strain ThNH-p12/93 (AF022438) |
| 724) | dengu65_20mers_0MM.txt | Dengue virus type 2 strain ThNH-p12/93 (AF022438) |
| 725) | dengu65_20mers_1MM.txt | Dengue virus type 2 strain ThNH-p12/93 (AF022438) |
| 726) | dengu65_20mers_2MM.txt | Dengue virus type 2 strain ThNH-p12/93 (AF022438) |
| 727) | dengu65_21mers_0MM.txt | Dengue virus type 2 strain ThNH-p12/93 (AF022438) |
| 728) | dengu65_21mers_1MM.txt | Dengue virus type 2 strain ThNH-p12/93 (AF022438) |
| 729) | dengu65_21mers_2MM.txt | Dengue virus type 2 strain ThNH-p12/93 (AF022438) |
| 730) | dengu65_22mers_0MM.txt | Dengue virus type 2 strain ThNH-p12/93 (AF022438) |
| 731) | dengu65_22mers_1MM.txt | Dengue virus type 2 strain ThNH-p12/93 (AF022438) |
| 732) | dengu65_22mers_2MM.txt | Dengue virus type 2 strain ThNH-p12/93 (AF022438) |
| 733) | dengu66_19mers_0MM.txt | Dengue virus type 2 strain ThNH-p14/93 (AF022439) |
| 734) | dengu66_19mers_1MM.txt | Dengue virus type 2 strain ThNH-p14/93 (AF022439) |
| 735) | dengu66_19mers_2MM.txt | Dengue virus type 2 strain ThNH-p14/93 (AF022439) |
| 736) | dengu66_20mers_0MM.txt | Dengue virus type 2 strain ThNH-p14/93 (AF022439) |
| 737) | dengu66_20mers_1MM.txt | Dengue virus type 2 strain ThNH-p14/93 (AF022439) |
| 738) | dengu66_20mers_2MM.txt | Dengue virus type 2 strain ThNH-p14/93 (AF022439) |
| 739) | dengu66_21mers_0MM.txt | Dengue virus type 2 strain ThNH-p14/93 (AF022439) |
| 740) | dengu66_21mers_1MM.txt | Dengue virus type 2 strain ThNH-p14/93 (AF022439) |
| 741) | dengu66_21mers_2MM.txt | Dengue virus type 2 strain ThNH-p14/93 (AF022439) |
| 742) | dengu66_22mers_0MM.txt | Dengue virus type 2 strain ThNH-p14/93 (AF022439) |
| 743) | dengu66_22mers_1MM.txt | Dengue virus type 2 strain ThNH-p14/93 (AF022439) |
| 744) | dengu66_22mers_2MM.txt | Dengue virus type 2 strain ThNH-p14/93 (AF022439) |
| 745) | dengu67_19mers_0MM.txt | Dengue virus type 2 strain ThNH-p16/93 (AF022440) |
| 746) | dengu67_19mers_1MM.txt | Dengue virus type 2 strain ThNH-p16/93 (AF022440) |
| 747) | dengu67_19mers_2MM.txt | Dengue virus type 2 strain ThNH-p16/93 (AF022440) |
| 748) | dengu67_20mers_0MM.txt | Dengue virus type 2 strain ThNH-p16/93 (AF022440) |
| 749) | dengu67_20mers_1MM.txt | Dengue virus type 2 strain ThNH-p16/93 (AF022440) |
| 750) | dengu67_20mers_2MM.txt | Dengue virus type 2 strain ThNH-p16/93 (AF022440) |
| 751) | dengu67_21mers_0MM.txt | Dengue virus type 2 strain ThNH-p16/93 (AF022440) |
| 752) | dengu67_21mers_1MM.txt | Dengue virus type 2 strain ThNH-p16/93 (AF022440) |
| 753) | dengu67_21mers_2MM.txt | Dengue virus type 2 strain ThNH-p16/93 (AF022440) |
| 754) | dengu67_22mers_0MM.txt | Dengue virus type 2 strain ThNH-p16/93 (AF022440) |
| 755) | dengu67_22mers_1MM.txt | Dengue virus type 2 strain ThNH-p16/93 (AF022440) |
| 756) | dengu67_22mers_2MM.txt | Dengue virus type 2 strain ThNH-p16/93 (AF022440) |
| 757) | dengu68_19mers_0MM.txt | Dengue virus type 2 strain ThNH-p36/93 (AF022441) |
| 758) | dengu68_19mers_1MM.txt | Dengue virus type 2 strain ThNH-p36/93 (AF022441) |
| 759) | dengu68_19mers_2MM.txt | Dengue virus type 2 strain ThNH-p36/93 (AF022441) |
| 760) | dengu68_20mers_0MM.txt | Dengue virus type 2 strain ThNH-p36/93 (AF022441) |
| 761) | dengu68_20mers_1MM.txt | Dengue virus type 2 strain ThNH-p36/93 (AF022441) |
| 762) | dengu68_20mers_2MM.txt | Dengue virus type 2 strain ThNH-p36/93 (AF022441) |
| 763) | dengu68_21mers_0MM.txt | Dengue virus type 2 strain ThNH-p36/93 (AF022441) |
| 764) | dengu68_21mers_1MM.txt | Dengue virus type 2 strain ThNH-p36/93 (AF022441) |
| 765) | dengu68_21mers_2MM.txt | Dengue virus type 2 strain ThNH-p36/93 (AF022441) |
| 766) | dengu68_22mers_0MM.txt | Dengue virus type 2 strain ThNH-p36/93 (AF022441) |
| 767) | dengu68_22mers_1MM.txt | Dengue virus type 2 strain ThNH-p36/93 (AF022441) |
| 768) | dengu68_22mers_2MM.txt | Dengue virus type 2 strain ThNH-p36/93 (AF022441) |
| 769) | dengu69_19mers_0MM.txt | Dengue virus type 2 China isolate 04 (AF119661) |
| 770) | dengu69_19mers_1MM.txt | Dengue virus type 2 China isolate 04 (AF119661) |
| 771) | dengu69_19mers_2MM.txt | Dengue virus type 2 China isolate 04 (AF119661) |
| 772) | dengu69_20mers_0MM.txt | Dengue virus type 2 China isolate 04 (AF119661) |
| 773) | dengu69_20mers_1MM.txt | Dengue virus type 2 China isolate 04 (AF119661) |
| 774) | dengu69_20mers_2MM.txt | Dengue virus type 2 China isolate 04 (AF119661) |
| 775) | dengu69_21mers_0MM.txt | Dengue virus type 2 China isolate 04 (AF119661) |
| 776) | dengu69_21mers_1MM.txt | Dengue virus type 2 China isolate 04 (AF119661) |
| 777) | dengu69_21mers_2MM.txt | Dengue virus type 2 China isolate 04 (AF119661) |
| 778) | dengu69_22mers_0MM.txt | Dengue virus type 2 China isolate 04 (AF119661) |
| 779) | dengu69_22mers_1MM.txt | Dengue virus type 2 China isolate 04 (AF119661) |
| 780) | dengu69_22mers_2MM.txt | Dengue virus type 2 China isolate 04 (AF119661) |
| 781) | dengu7_19mers_0MM.txt | Dengue virus type 2 strain New Guinea C/PUO-218 hybrid polyprotein (AF038402) |
| 782) | dengu7_19mers_1MM.txt | Dengue virus type 2 strain New Guinea C/PUO-218 hybrid polyprotein (AF038402) |
| 783) | dengu7_19mers_2MM.txt | Dengue virus type 2 strain New Guinea C/PUO-218 hybrid polyprotein (AF038402) |
| 784) | dengu7_20mers_0MM.txt | Dengue virus type 2 strain New Guinea C/PUO-218 hybrid polyprotein (AF038402) |
| 785) | dengu7_20mers_1MM.txt | Dengue virus type 2 strain New Guinea C/PUO-218 hybrid polyprotein (AF038402) |
| 786) | dengu7_20mers_2MM.txt | Dengue virus type 2 strain New Guinea C/PUO-218 hybrid polyprotein (AF038402) |
| 787) | dengu7_21mers_0MM.txt | Dengue virus type 2 strain New Guinea C/PUO-218 hybrid polyprotein (AF038402) |
| 788) | dengu7_21mers_1MM.txt | Dengue virus type 2 strain New Guinea C/PUO-218 hybrid polyprotein (AF038402) |
| 789) | dengu7_21mers_2MM.txt | Dengue virus type 2 strain New Guinea C/PUO-218 hybrid |
| 790) | dengu7_22mers_0MM.txt | Dengue virus type 2 strain New Guinea C/PUO-218 hybrid polyprotein (AF038402) |
| 791) | dengu7_22mers_1MM.txt | Dengue virus type 2 strain New Guinea C/PUO-218 hybrid polyprotein (AF038402) |
| 792) | dengu7_22mers_2MM.txt | Dengue virus type 2 strain New Guinea C/PUO-218 hybrid polyprotein (AF038402) |
| 793) | dengu70_19mers_0MM.txt | Dengue virus type 2 strain 44 (AF204177) |
| 794) | dengu70_19mers_1MM.txt | Dengue virus type 2 strain 44 (AF204177) |
| 795) | dengu70_19mers_2MM.txt | Dengue virus type 2 strain 44 (AF204177) |
| 796) | dengu70_20mers_0MM.txt | Dengue virus type 2 strain 44 (AF204177) |
| 797) | dengu70_20mers_1MM.txt | Dengue virus type 2 strain 44 (AF204177) |
| 798) | dengu70_20mers_2MM.txt | Dengue virus type 2 strain 44 (AF204177) |
| 799) | dengu70_21mers_0MM.txt | Dengue virus type 2 strain 44 (AF204177) |
| 800) | dengu70_21mers_1MM.txt | Dengue virus type 2 strain 44 (AF204177) |
| 801) | dengu70_21mers_2MM.txt | Dengue virus type 2 strain 44 (AF204177) |
| 802) | dengu70_22mers_0MM.txt | Dengue virus type 2 strain 44 (AF204177) |
| 803) | dengu70_22mers_1MM.txt | Dengue virus type 2 strain 44 (AF204177) |
| 804) | dengu70_22mers_2MM.txt | Dengue virus type 2 strain 44 (AF204177) |
| 805) | dengu71_19mers_0MM.txt | Dengue virus type 2 strain 43 (AF204178) |
| 806) | dengu71_19mers_1MM.txt | Dengue virus type 2 strain 43 (AF204178) |
| 807) | dengu71_19mers_2MM.txt | Dengue virus type 2 strain 43 (AF204178) |
| 808) | dengu71_20mers_0MM.txt | Dengue virus type 2 strain 43 (AF204178) |
| 809) | dengu71_20mers_1MM.txt | Dengue virus type 2 strain 43 (AF204178) |
| 810) | dengu71_20mers_2MM.txt | Dengue virus type 2 strain 43 (AF204178) |
| 811) | dengu71_21mers_0MM.txt | Dengue virus type 2 strain 43 (AF204178) |
| 812) | dengu71_21mers_1MM.txt | Dengue virus type 2 strain 43 (AF204178) |
| 813) | dengu71_21mers_2MM.txt | Dengue virus type 2 strain 43 (AF204178) |
| 814) | dengu71_22mers_0MM.txt | Dengue virus type 2 strain 43 (AF204178) |
| 815) | dengu71_22mers_1MM.txt | Dengue virus type 2 strain 43 (AF204178) |
| 816) | dengu71_22mers_2MM.txt | Dengue virus type 2 strain 43 (AF204178) |
| 817) | dengu72_19mers_0MM.txt | Dengue virus type 2 strain ThNH29/93 (AF169678) |
| 818) | dengu72_19mers_1MM.txt | Dengue virus type 2 strain ThNH29/93 (AF169678) |
| 819) | dengu72_19mers_2MM.txt | Dengue virus type 2 strain ThNH29/93 (AF169678) |
| 820) | dengu72_20mers_0MM.txt | Dengue virus type 2 strain ThNH29/93 (AF169678) |
| 821) | dengu72_20mers_1MM.txt | Dengue virus type 2 strain ThNH29/93 (AF169678) |
| 822) | dengu72_20mers_2MM.txt | Dengue virus type 2 strain ThNH29/93 (AF169678) |
| 823) | dengu72_21mers_0MM.txt | Dengue virus type 2 strain ThNH29/93 (AF169678) |
| 824) | dengu72_21mers_1MM.txt | Dengue virus type 2 strain ThNH29/93 (AF169678) |
| 825) | dengu72_21mers_2MM.txt | Dengue virus type 2 strain ThNH29/93 (AF169678) |
| 826) | dengu72_22mers_0MM.txt | Dengue virus type 2 strain ThNH29/93 (AF169678) |
| 827) | dengu72_22mers_1MM.txt | Dengue virus type 2 strain ThNH29/93 (AF169678) |
| 828) | dengu72_22mers_2MM.txt | Dengue virus type 2 strain ThNH29/93 (AF169678) |
| 829) | dengu73_19mers_0MM.txt | Dengue virus type 2 strain ThNH36/93 (AF169679) |
| 830) | dengu73_19mers_1MM.txt | Dengue virus type 2 strain ThNH36/93 (AF169679) |
| 831) | dengu73_19mers_2MM.txt | Dengue virus type 2 strain ThNH36/93 (AF169679) |
| 832) | dengu73_20mers_0MM.txt | Dengue virus type 2 strain ThNH36/93 (AF169679) |
| 833) | dengu73_20mers_1MM.txt | Dengue virus type 2 strain ThNH36/93 (AF169679) |
| 834) | dengu73_20mers_2MM.txt | Dengue virus type 2 strain ThNH36/93 (AF169679) |
| 835) | dengu73_21mers_0MM.txt | Dengue virus type 2 strain ThNH36/93 (AF169679) |
| 836) | dengu73_21mers_1MM.txt | Dengue virus type 2 strain ThNH36/93 (AF169679) |
| 837) | dengu73_21mers_2MM.txt | Dengue virus type 2 strain ThNH36/93 (AF169679) |
| 838) | dengu73_22mers_0MM.txt | Dengue virus type 2 strain ThNH36/93 (AF169679) |
| 839) | dengu73_22mers_1MM.txt | Dengue virus type 2 strain ThNH36/93 (AF169679) |
| 840) | dengu73_22mers_2MM.txt | Dengue virus type 2 strain ThNH36/93 (AF169679) |
| 841) | dengu74_19mers_0MM.txt | Dengue virus type 2 strain ThNH45/93 (AF169680) |
| 842) | dengu74_19mers_1MM.txt | Dengue virus type 2 strain ThNH45/93 (AF169680) |
| 843) | dengu74_19mers_2MM.txt | Dengue virus type 2 strain ThNH45/93 (AF169680) |
| 844) | dengu74_20mers_0MM.txt | Dengue virus type 2 strain ThNH45/93 (AF169680) |
| 845) | dengu74_20mers_1MM.txt | Dengue virus type 2 strain ThNH45/93 (AF169680) |
| 846) | dengu74_20mers_2MM.txt | Dengue virus type 2 strain ThNH45/93 (AF169680) |
| 847) | dengu74_21mers_0MM.txt | Dengue virus type 2 strain ThNH45/93 (AF169680) |
| 848) | dengu74_21mers_1MM.txt | Dengue virus type 2 strain ThNH45/93 (AF169680) |
| 849) | dengu74_21mers_2MM.txt | Dengue virus type 2 strain ThNH45/93 (AF169680) |
| 850) | dengu74_22mers_0MM.txt | Dengue virus type 2 strain ThNH45/93 (AF169680) |
| 851) | dengu74_22mers_1MM.txt | Dengue virus type 2 strain ThNH45/93 (AF169680) |
| 852) | dengu74_22mers_2MM.txt | Dengue virus type 2 strain ThNH45/93 (AF169680) |
| 853) | dengu75_19mers_0MM.txt | Dengue virus type 2 strain ThNH55/93 (AF169681) |
| 854) | dengu75_19mers_1MM.txt | Dengue virus type 2 strain ThNH55/93 (AF169681) |
| 855) | dengu75_19mers_2MM.txt | Dengue virus type 2 strain ThNH55/93 (AF169681) |
| 856) | dengu75_20mers_0MM.txt | Dengue virus type 2 strain ThNH55/93 (AF169681) |
| 857) | dengu75_20mers_1MM.txt | Dengue virus type 2 strain ThNH55/93 (AF169681) |
| 858) | dengu75_20mers_2MM.txt | Dengue virus type 2 strain ThNH55/93 (AF169681) |
| 859) | dengu75_21mers_0MM.txt | Dengue virus type 2 strain ThNH55/93 (AF169681) |
| 860) | dengu75_21mers_1MM.txt | Dengue virus type 2 strain ThNH55/93 (AF169681) |
| 861) | dengu75_21mers_2MM.txt | Dengue virus type 2 strain ThNH55/93 (AF169681) |
| 862) | dengu75_22mers_0MM.txt | Dengue virus type 2 strain ThNH55/93 (AF169681) |
| 863) | dengu75_22mers_1MM.txt | Dengue virus type 2 strain ThNH55/93 (AF169681) |
| 864) | dengu75_22mers_2MM.txt | Dengue virus type 2 strain ThNH55/93 (AF169681) |
| 865) | dengu76_19mers_0MM.txt | Dengue virus type 2 strain ThNH54/93 (AF169682) |
| 866) | dengu76_19mers_1MM.txt | Dengue virus type 2 strain ThNH54/93 (AF169682) |
| 867) | dengu76_19mers_2MM.txt | Dengue virus type 2 strain ThNH54/93 (AF169682) |
| 868) | dengu76_20mers_0MM.txt | Dengue virus type 2 strain ThNH54/93 (AF169682) |
| 869) | dengu76_20mers_1MM.txt | Dengue virus type 2 strain ThNH54/93 (AF169682) |
| 870) | dengu76_20mers_2MM.txt | Dengue virus type 2 strain ThNH54/93 (AF169682) |
| 871) | dengu76_21mers_0MM.txt | Dengue virus type 2 strain ThNH54/93 (AF169682) |
| 872) | dengu76_21mers_1MM.txt | Dengue virus type 2 strain ThNH54/93 (AF169682) |
| 873) | dengu76_21mers_2MM.txt | Dengue virus type 2 strain ThNH54/93 (AF169682) |
| 874) | dengu76_22mers_0MM.txt | Dengue virus type 2 strain ThNH54/93 (AF169682) |
| 875) | dengu76_22mers_1MM.txt | Dengue virus type 2 strain ThNH54/93 (AF169682) |
| 876) | dengu76_22mers_2MM.txt | Dengue virus type 2 strain ThNH54/93 (AF169682) |
| 877) | dengu77_19mers_0MM.txt | Dengue virus type 2 strain ThNH62/93 (AF169683) |
| 878) | dengu77_19mers_1MM.txt | Dengue virus type 2 strain ThNH62/93 (AF169683) |
| 879) | dengu77_19mers_2MM.txt | Dengue virus type 2 strain ThNH62/93 (AF169683) |
| 880) | dengu77_20mers_0MM.txt | Dengue virus type 2 strain ThNH62/93 (AF169683) |
| 881) | dengu77_20mers_1MM.txt | Dengue virus type 2 strain ThNH62/93 (AF169683) |
| 882) | dengu77_20mers_2MM.txt | Dengue virus type 2 strain ThNH62/93 (AF169683) |
| 883) | dengu77_21mers_0MM.txt | Dengue virus type 2 strain ThNH62/93 (AF169683) |
| 884) | dengu77_21mers_1MM.txt | Dengue virus type 2 strain ThNH62/93 (AF169683) |
| 885) | dengu77_21mers_2MM.txt | Dengue virus type 2 strain ThNH62/93 (AF169683) |
| 886) | dengu77_22mers_0MM.txt | Dengue virus type 2 strain ThNH62/93 (AF169683) |
| 887) | dengu77_22mers_1MM.txt | Dengue virus type 2 strain ThNH62/93 (AF169683) |
| 888) | dengu77_22mers_2MM.txt | Dengue virus type 2 strain ThNH62/93 (AF169683) |
| 889) | dengu78_19mers_0MM.txt | Dengue virus type 2 strain ThNH63/93 (AF169684) |
| 890) | dengu78_19mers_1MM.txt | Dengue virus type 2 strain ThNH63/93 (AF169684) |
| 891) | dengu78_19mers_2MM.txt | Dengue virus type 2 strain ThNH63/93 (AF169684) |
| 892) | dengu78_20mers_0MM.txt | Dengue virus type 2 strain ThNH63/93 (AF169684) |
| 893) | dengu78_20mers_1MM.txt | Dengue virus type 2 strain ThNH63/93 (AF169684) |
| 894) | dengu78_20mers_2MM.txt | Dengue virus type 2 strain ThNH63/93 (AF169684) |
| 895) | dengu78_21mers_0MM.txt | Dengue virus type 2 strain ThNH63/93 (AF169684) |
| 896) | dengu78_21mers_1MM.txt | Dengue virus type 2 strain ThNH63/93 (AF169684) |
| 897) | dengu78_21mers_2MM.txt | Dengue virus type 2 strain ThNH63/93 (AF169684) |
| 898) | dengu78_22mers_0MM.txt | Dengue virus type 2 strain ThNH63/93 (AF169684) |
| 899) | dengu78_22mers_1MM.txt | Dengue virus type 2 strain ThNH63/93 (AF169684) |
| 900) | dengu78_22mers_2MM.txt | Dengue virus type 2 strain ThNH63/93 (AF169684) |
| 901) | dengu79_19mers_0MM.txt | Dengue virus type 2 strain ThNH69/93 (AF169685) |
| 902) | dengu79_19mers_1MM.txt | Dengue virus type 2 strain ThNH69/93 (AF169685) |
| 903) | dengu79_19mers_2MM.txt | Dengue virus type 2 strain ThNH69/93 (AF169685) |
| 904) | dengu79_20mers_0MM.txt | Dengue virus type 2 strain ThNH69/93 (AF169685) |
| 905) | dengu79_20mers_1MM.txt | Dengue virus type 2 strain ThNH69/93 (AF169685) |
| 906) | dengu79_20mers_2MM.txt | Dengue virus type 2 strain ThNH69/93 (AF169685) |
| 907) | dengu79_21mers_0MM.txt | Dengue virus type 2 strain ThNH69/93 (AF169685) |
| 908) | dengu79_21mers_1MM.txt | Dengue virus type 2 strain ThNH69/93 (AF169685) |
| 909) | dengu79_21mers_2MM.txt | Dengue virus type 2 strain ThNH69/93 (AF169685) |
| 910) | dengu79_22mers_0MM.txt | Dengue virus type 2 strain ThNH69/93 (AF169685) |
| 911) | dengu79_22mers_1MM.txt | Dengue virus type 2 strain ThNH69/93 (AF169685) |
| 912) | dengu79_22mers_2MM.txt | Dengue virus type 2 strain ThNH69/93 (AF169685) |
| 913) | dengu8_19mers_0MM.txt | Dengue virus type 2 strain New Guinea C polyprotein mRNA (AF038403) |
| 914) | dengu8_19mers_1MM.txt | Dengue virus type 2 strain New Guinea C polyprotein mRNA |
| | | (AF038403) |
| 915) | dengu8_19mers_2MM.txt | Dengue virus type 2 strain New Guinea C polyprotein mRNA |
| | | (AF038403) |
| 916) | dengu8_20mers_0MM.txt | Dengue virus type 2 strain New Guinea C polyprotein mRNA |
| | | (AF038403) |
| 917) | dengu8_20mers_1MM.txt | Dengue virus type 2 strain New Guinea C polyprotein mRNA |
| | | (AF038403) |
| 918) | dengu8_20mers_2MM.txt | Dengue virus type 2 strain New Guinea C polyprotein mRNA |
| | | (AF038403) |
| 919) | dengu8_21mers_0MM.txt | Dengue virus type 2 strain New Guinea C polyprotein mRNA |
| | | (AF038403) |
| 920) | dengu8_21mers_1MM.txt | Dengue virus type 2 strain New Guinea C polyprotein mRNA |
| | | (AF038403) |
| 921) | dengu8_21mers_2MM.txt | Dengue virus type 2 strain New Guinea C polyprotein mRNA |
| | | (AF038403) |
| 922) | dengu8_22mers_0MM.txt | Dengue virus type 2 strain New Guinea C polyprotein mRNA |
| | | (AF038403) |
| 923) | dengu8_22mers_1MM.txt | Dengue virus type 2 strain New Guinea C polyprotein mRNA |
| | | (AF038403) |
| 924) | dengu8_22mers_2MM.txt | Dengue virus type 2 strain New Guinea C polyprotein mRNA |
| | | (AF038403) |
| 925) | dengu80_19mers_0MM.txt | Dengue virus type 2 strain ThNH73/93 (AF169686) |
| 926) | dengu80_19mers_1MM.txt | Dengue virus type 2 strain ThNH73/93 (AF169686) |
| 927) | dengu80_19mers_2MM.txt | Dengue virus type 2 strain ThNH73/93 (AF169686) |
| 928) | dengu80_20mers_0MM.txt | Dengue virus type 2 strain ThNH73/93 (AF169686) |
| 929) | dengu80_20mers_1MM.txt | Dengue virus type 2 strain ThNH73/93 (AF169686) |
| 930) | dengu80_20mers_2MM.txt | Dengue virus type 2 strain ThNH73/93 (AF169686) |
| 931) | dengu80_21mers_0MM.txt | Dengue virus type 2 strain ThNH73/93 (AF169686) |
| 932) | dengu80_21mers_1MM.txt | Dengue virus type 2 strain ThNH73/93 (AF169686) |
| 933) | dengu80_21mers_2MM.txt | Dengue virus type 2 strain ThNH73/93 (AF169686) |
| 934) | dengu80_22mers_0MM.txt | Dengue virus type 2 strain ThNH73/93 (AF169686) |
| 935) | dengu80_22mers_1MM.txt | Dengue virus type 2 strain ThNH73/93 (AF169686) |
| 936) | dengu80_22mers_2MM.txt | Dengue virus type 2 strain ThNH73/93 (AF169686) |
| 937) | dengu81_19mers_0MM.txt | Dengue virus type 2 strain ThNH76/93 (AF169687) |
| 938) | dengu81_19mers_1MM.txt | Dengue virus type 2 strain ThNH76/93 (AF169687) |
| 939) | dengu81_19mers_2MM.txt | Dengue virus type 2 strain ThNH76/93 (AF169687) |
| 940) | dengu81_20mers_0MM.txt | Dengue virus type 2 strain ThNH76/93 (AF169687) |
| 941) | dengu81_20mers_1MM.txt | Dengue virus type 2 strain ThNH76/93 (AF169687) |
| 942) | dengu81_20mers_2MM.txt | Dengue virus type 2 strain ThNH76/93 (AF169687) |
| 943) | dengu81_21mers_0MM.txt | Dengue virus type 2 strain ThNH76/93 (AF169687) |
| 944) | dengu81_21mers_1MM.txt | Dengue virus type 2 strain ThNH76/93 (AF169687) |
| 945) | dengu81_21mers_2MM.txt | Dengue virus type 2 strain ThNH76/93 (AF169687) |
| 946) | dengu81_22mers_0MM.txt | Dengue virus type 2 strain ThNH76/93 (AF169687) |
| 947) | dengu81_22mers_1MM.txt | Dengue virus type 2 strain ThNH76/93 (AF169687) |
| 948) | dengu81_22mers_2MM.txt | Dengue virus type 2 strain ThNH76/93 (AF169687) |
| 949) | dengu82_19mers_0MM.txt | Dengue virus type 2 strain ThNH81/93 (AF169688) |
| 950) | dengu82_19mers_1MM.txt | Dengue virus type 2 strain ThNH81/93 (AF169688) |
| 951) | dengu82_19mers_2MM.txt | Dengue virus type 2 strain ThNH81/93 (AF169688) |
| 952) | dengu82_20mers_0MM.txt | Dengue virus type 2 strain ThNH81/93 (AF169688) |
| 953) | dengu82_20mers_1MM.txt | Dengue virus type 2 strain ThNH81/93 (AF169688) |
| 954) | dengu82_20mers_2MM.txt | Dengue virus type 2 strain ThNH81/93 (AF169688) |
| 955) | dengu82_21mers_0MM.txt | Dengue virus type 2 strain ThNH81/93 (AF169688) |
| 956) | dengu82_21mers_1MM.txt | Dengue virus type 2 strain ThNH81/93 (AF169688) |
| 957) | dengu82_21mers_2MM.txt | Dengue virus type 2 strain ThNH81/93 (AF169688) |
| 958) | dengu82_22mers_0MM.txt | Dengue virus type 2 strain ThNH81/93 (AF169688) |
| 959) | dengu82_22mers_1MM.txt | Dengue virus type 2 strain ThNH81/93 (AF169688) |
| 960) | dengu82_22mers_2MM.txt | Dengue virus type 2 strain ThNH81/93 (AF169688) |
| 961) | dengu83_19mers_0MM.txt | Dengue virus type 1 strain FGA/NA d1d (AF226686) |
| 962) | dengu83_19mers_1MM.txt | Dengue virus type 1 strain FGA/NA d1d (AF226686) |
| 963) | dengu83_19mers_2MM.txt | Dengue virus type 1 strain FGA/NA d1d (AF226686) |
| 964) | dengu83_20mers_0MM.txt | Dengue virus type 1 strain FGA/NA d1d (AF226686) |
| 965) | dengu83_20mers_1MM.txt | Dengue virus type 1 strain FGA/NA d1d (AF226686) |
| 966) | dengu83_20mers_2MM.txt | Dengue virus type 1 strain FGA/NA d1d (AF226686) |
| 967) | dengu83_21mers_0MM.txt | Dengue virus type 1 strain FGA/NA d1d (AF226686) |
| 968) | dengu83_21mers_1MM.txt | Dengue virus type 1 strain FGA/NA d1d (AF226686) |
| 969) | dengu83_21mers_2MM.txt | Dengue virus type 1 strain FGA/NA d1d (AF226686) |
| 970) | dengu83_22mers_0MM.txt | Dengue virus type 1 strain FGA/NA d1d (AF226686) |
| 971) | dengu83_22mers_1MM.txt | Dengue virus type 1 strain FGA/NA d1d (AF226686) |
| 972) | dengu83_22mers_2MM.txt | Dengue virus type 1 strain FGA/NA d1d (AF226686) |
| 973) | dengu84_19mers_0MM.txt | Dengue virus type 2 strain FJ-10 (AF276619) |
| 974) | dengu84_19mers_1MM.txt | Dengue virus type 2 strain FJ-10 (AF276619) |
| 975) | dengu84_19mers_2MM.txt | Dengue virus type 2 strain FJ-10 (AF276619) |
| 976) | dengu84_20mers_0MM.txt | Dengue virus type 2 strain FJ-10 (AF276619) |
| 977) | dengu84_20mers_1MM.txt | Dengue virus type 2 strain FJ-10 (AF276619) |
| 978) | dengu84_20mers_2MM.txt | Dengue virus type 2 strain FJ-10 (AF276619) |
| 979) | dengu84_21mers_0MM.txt | Dengue virus type 2 strain FJ-10 (AF276619) |
| 980) | dengu84_21mers_1MM.txt | Dengue virus type 2 strain FJ-10 (AF276619) |
| 981) | dengu84_21mers_2MM.txt | Dengue virus type 2 strain FJ-10 (AF276619) |
| 982) | dengu84_22mers_0MM.txt | Dengue virus type 2 strain FJ-10 (AF276619) |
| 983) | dengu84_22mers_1MM.txt | Dengue virus type 2 strain FJ-10 (AF276619) |
| 984) | dengu84_22mers_2MM.txt | Dengue virus type 2 strain FJ-10 (AF276619) |
| 985) | dengu9_19mers_0MM.txt | Dengue virus type 2 strain K0008 polyprotein gene (AF100459) |
| 986) | dengu9_19mers_1MM.txt | Dengue virus type 2 strain K0008 polyprotein gene (AF100459) |
| 987) | dengu9_19mers_2MM.txt | Dengue virus type 2 strain K0008 polyprotein gene (AF100459) |
| 988) | dengu9_20mers_0MM.txt | Dengue virus type 2 strain K0008 polyprotein gene (AF100459) |
| 989) | dengu9_20mers_1MM.txt | Dengue virus type 2 strain K0008 polyprotein gene (AF100459) |
| 990) | dengu9_20mers_2MM.txt | Dengue virus type 2 strain K0008 polyprotein gene (AF100459) |
| 991) | dengu9_21mers_0MM.txt | Dengue virus type 2 strain K0008 polyprotein gene (AF100459) |
| 992) | dengu9_21mers_1MM.txt | Dengue virus type 2 strain K0008 polyprotein gene (AF100459) |
| 993) | dengu9_21mers_2MM.txt | Dengue virus type 2 strain K0008 polyprotein gene (AF100459) |
| 994) | dengu9_22mers_0MM.txt | Dengue virus type 2 strain K0008 polyprotein gene (AF100459) |
| 995) | dengu9_22mers_1MM.txt | Dengue virus type 2 strain K0008 polyprotein gene (AF100459) |
| 996) | dengu9_22mers_2MM.txt | Dengue virus type 2 strain K0008 polyprotein gene (AF100459) |
| 997) | Dengue(NC_001474)_16mers_0MM.txt | Dengue (NC 001474) |
| 998) | Dengue(NC_001474)_16mers_1MM.txt | Dengue (NC 001474) |
| 999) | Dengue(NC_001474)_16mers_2MM.txt | Dengue (NC 001474) |
| 1000) | Dengue(NC_001474)_17mers_0MM.txt | Dengue (NC 001474) |
| 1001) | Dengue(NC_001474)_17mers_1MM.txt | Dengue (NC 001474) |
| 1002) | Dengue(NC_001474)_17mers_2MM.txt | Dengue (NC 001474) |
| 1003) | Dengue(NC_001474)_18mers_0MM.txt | Dengue (NC 001474) |
| 1004) | Dengue(NC_001474)_18mers_1MM.txt | Dengue (NC 001474) |
| 1005) | Dengue(NC_001474)_18mers_2MM.txt | Dengue (NC 001474) |
| 1006) | DV_t1(M87512)_16mers_0MM.txt | DV t1 (M87512) |
| 1007) | DV_t1(M87512)_16mers_1MM.txt | DV t1 (M87512) |
| 1008) | DV_t1(M87512)_16mers_2MM.txt | DV t1 (M87512) |
| 1009) | DV_t1(M87512)_17mers_0MM.txt | DV t1 (M87512) |
| 1010) | DV_t1(M87512)_17mers_1MM.txt | DV t1 (M87512) |
| 1011) | DV_t1(M87512)_17mers_2MM.txt | DV t1 (M87512) |
| 1012) | DV_t1(M87512)_18mers_0MM.txt | DV t1 (M87512) |
| 1013) | DV_t1(M87512)_18mers_1MM.txt | DV t1 (M87512) |
| 1014) | DV_t1(M87512)_18mers_2MM.txt | DV t1 (M87512) |
| 1015) | DV_t1_Abidjan_strain_polyprotein_gene(AF298807)_16mers_0MM.txt | DV t1 Abidjan strain |
| | | polyprotein gene (AF298807) |
| 1016) | DV_t1_Abidjan_strain_polyprotein_gene(AF298807)_16mers_1MM.txt | DV t1 Abidjan strain |
| | | polyprotein gene (AF298807) |
| 1017) | DV_t1_Abidjan_strain_polyprotein_gene(AF298807)_16mers_2MM.txt | DV t1 Abidjan strain |
| | | polyprotein gene (AF298807) |
| 1018) | DV_t1_Abidjan_strain_polyprotein_gene(AF298807)_17mers_0MM.txt | DV t1 Abidjan strain |
| | | polyprotein gene (AF298807) |
| 1019) | DV_t1_Abidjan_strain_polyprotein_gene(AF298807)_17mers_1MM.txt | DV t1 Abidjan strain |
| | | polyprotein gene (AF298807) |
| 1020) | DV_t1_Abidjan_strain_polyprotein_gene(AF298807)_17mers_2MM.txt | DV t1 Abidjan strain |
| | | polyprotein gene (AF298807) |
| 1021) | DV_t1_Abidjan_strain_polyprotein_gene(AF298807)_18mers_0MM.txt | DV t1 Abidjan strain |
| | | polyprotein gene (AF298807) |
| 1022) | DV_t1_Abidjan_strain_polyprotein_gene(AF298807)_18mers_1MM.txt | DV t1 Abidjan strain |
| | | polyprotein gene (AF298807) |
| 1023) | DV_t1_Abidjan_strain_polyprotein_gene(AF298807)_18mers_2MM.txt | DV t1 Abidjan strain |
| | | polyprotein gene (AF298807) |
| 1024) | DV_t1_clone_45AZ5(U88536)_16mers_0MM.txt | DV t1 clone 45AZ5 (U88536) |
| 1025) | DV_t1_clone_45AZ5(U88536)_16mers_1MM.txt | DV t1 clone 45AZ5 (U88536) |
| 1026) | DV_t1_clone_45AZ5(U88536)_16mers_2MM.txt | DV t1 clone 45AZ5 (U88536) |
| 1027) | DV_t1_clone_45AZ5(U88536)_17mers_0MM.txt | DV t1 clone 45AZ5 (U88536) |
| 1028) | DV_t1_clone_45AZ5(U88536)_17mers_1MM.txt | DV t1 clone 45AZ5 (U88536) |
| 1029) | DV_t1_clone_45AZ5(U88536)_17mers_2MM.txt | DV t1 clone 45AZ5 (U88536) |
| 1030) | DV_t1_clone_45AZ5(U88536)_18mers_0MM.txt | DV t1 clone 45AZ5 (U88536) |
| 1031) | DV_t1_clone_45AZ5(U88536)_18mers_1MM.txt | DV t1 clone 45AZ5 (U88536) |
| 1032) | DV_t1_clone_45AZ5(U88536)_18mers_2MM.txt | DV t1 clone 45AZ5 (U88536) |
| 1033) | DV_t1_clone_45AZ5-PDK27(U88537)_16mers_0MM.txt | DV t1 clone 45AZ5-PDK27 |
| | | (U88537) |
| 1034) | DV_t1_clone_45AZ5-PDK27(U88537)_16mers_1MM.txt | DV t1 clone 45AZ5-PDK27 |
| | | (U88537) |
| 1035) | DV_t1_clone_45AZ5-PDK27(U88537)_16mers_2MM.txt | DV t1 clone 45AZ5-PDK27 |
| | | (U88537) |
| 1036) | DV_t1_clone_45AZ5-PDK27(U88537)_17mers_0MM.txt | DV t1 clone 45AZ5-PDK27 |
| | | (U88537) |
| 1037) | DV_t1_clone_45AZ5-PDK27(U88537)_17mers_1MM.txt | DV t1 clone 45AZ5-PDK27 |
| | | (U88537) |
| 1038) | DV_t1_clone_45AZ5-PDK27(U88537)_17mers_2MM.txt | DV t1 clone 45AZ5-PDK27 |
| | | (U88537) |
| 1039) | DV_t1_clone_45AZ5-PDK27(U88537)_18mers_0MM.txt | DV t1 clone 45AZ5-PDK27 |
| | | (U88537) |
| 1040) | DV_t1_clone_45AZ5-PDK27(U88537)_18mers_1MM.txt | DV t1 clone 45AZ5-PDK27 |
| | | (U88537) |
| 1041) | DV_t1_clone_45AZ5-PDK27(U88537)_18mers_2MM.txt | DV t1 clone 45AZ5-PDK27 |
| | | (U88537) |
| 1042) | DV_t1_clone_WestPac(U88535)_16mers_0MM.txt | DV t1 clone WestPac (U88535) |
| 1043) | DV_t1_clone_WestPac(U88535)_16mers_1MM.txt | DV t1 clone WestPac (U88535) |
| 1044) | DV_t1_clone_WestPac(U88535)_16mers_2MM.txt | DV t1 clone WestPac (U88535) |
| 1045) | DV_t1_clone_WestPac(U88535)_17mers_0MM.txt | DV t1 clone WestPac (U88535) |
| 1046) | DV_t1_clone_WestPac(U88535)_17mers_1MM.txt | DV t1 clone WestPac (U88535) |
| 1047) | DV_t1_clone_WestPac(U88535)_17mers_2MM.txt | DV t1 clone WestPac (U88535) |
| 1048) | DV_t1_clone_WestPac(U88535)_18mers_0MM.txt | DV t1 clone WestPac (U88535) |
| 1049) | DV_t1_clone_WestPac(U88535)_18mers_1MM.txt | DV t1 clone WestPac (U88535) |
| 1050) | DV_t1_clone_WestPac(U88535)_18mers_2MM.txt | DV t1 clone WestPac (U88535) |
| 1051) | DV_t1_Djibouti_strain_D1-H-IMTSSA-98-606_polyprotein_gene(AF298808)_16mers_0MM.txt | DV t1 Djibouti strain D1-H-IMTSSA-98-606 polyprotein gene (AF298808) |
| 1052) | DV_t1_Djibouti_strain_D1-H-IMTSSA-98-606_polyprotein_gene(AF298808)_16mers_1MM.txt | DV t1 Djibouti strain D1-H-IMTSSA-98-606 polyprotein gene (AF298808) |
| 1053) | DV_t1_Djibouti_strain_D1-H-IMTSSA-98-606_polyprotein_gene(AF298808)_16mers_2MM.txt | DV t1 Djibouti strain D1-H-IMTSSA-98-606 polyprotein gene (AF298808) |
| 1054) | DV_t1_Djibouti_strain_D1-H-IMTSSA-98-606_polyprotein_gene(AF298808)_17mers_0MM.txt | DV t1 Djibouti strain D1-H-IMTSSA-98-606 polyprotein gene (AF298808) |
| 1055) | DV_t1_Djibouti_strain_D1-H-IMTSSA-98-606_polyprotein_gene(AF298808)_17mers_1MM.txt | DV t1 Djibouti strain D1-H-IMTSSA-98-606 polyprotein gene (AF298808) |
| 1056) | DV_t1_Djibouti_strain_D1-H-IMTSSA-98-606_polyprotein_gene(AF298808)_17mers_2MM.txt | DV t1 Djibouti strain D1-H-IMTSSA-98-606 polyprotein gene (AF298808) |
| 1057) | DV_t1_Djibouti_strain_D1-H-IMTSSA-98-606_polyprotein_gene(AF298808)_18mers_0MM.txt | DV t1 Djibouti strain D1-H-IMTSSA-98-606 polyprotein gene (AF298808) |
| 1058) | DV_t1_Djibouti_strain_D1-H-IMTSSA-98-606_polyprotein_gene(AF298808)_18mers_1MM.txt | DV t1 Djibouti strain D1-H-IMTSSA-98-606 polyprotein gene (AF298808) |
| 1059) | DV_t1_Djibouti_strain_D1-H-IMTSSA-98-606_polyprotein_gene(AF298808)_18mers_2MM.txt | DV t1 Djibouti strain D1-H-IMTSSA-98-606 polyprotein gene (AF298808) |
| 1060) | DV_t1_from_Cambodia(AF309641)_16mers_0MM.txt | DV t1 from Cambodia (AF309641) |
| 1061) | DV_t1_from_Cambodia(AF309641)_16mers_1MM.txt | DV t1 from Cambodia (AF309641) |
| 1062) | DV_t1_from_Cambodia(AF309641)_16mers_2MM.txt | DV t1 from Cambodia (AF309641) |
| 1063) | DV_t1_from_Cambodia(AF309641)_17mers_0MM.txt | DV t1 from Cambodia (AF309641) |
| 1064) | DV_t1_from_Cambodia(AF309641)_17mers_1MM.txt | DV t1 from Cambodia (AF309641) |
| 1065) | DV_t1_from_Cambodia(AF309641)_17mers_2MM.txt | DV t1 from Cambodia (AF309641) |
| 1066) | DV_t1_from_Cambodia(AF309641)_18mers_0MM.txt | DV t1 from Cambodia (AF309641) |
| 1067) | DV_t1_from_Cambodia(AF309641)_18mers_1MM.txt | DV t1 from Cambodia (AF309641) |
| 1068) | DV_t1_from_Cambodia(AF309641)_18mers_2MM.txt | DV t1 from Cambodia (AF309641) |
| 1069) | DV_t1_from_Patent_W09322440(A75711)_16mers_0MM.txt | DV t1 from Patent W09322440 |
| | | (A75711) |
| 1070) | DV_t1_from_Patent_W09322440(A75711)_16mers_1MM.txt | DV t1 from Patent W09322440 |
| | | (A75711) |
| 1071) | DV_t1_from_Patent_W09322440(A75711)_16mers_2MM.txt | DV t1 from Patent W09322440 |
| | | (A75711) |
| 1072) | DV_t1_from_Patent_W09322440(A75711)_17mers_0MM.txt | DV t1 from Patent W09322440 |
| | | (A75711) |
| 1073) | DV_t1_from_Patent_W09322440(A75711)_17mers_1MM.txt | DV t1 from Patent W09322440 |
| | | (A75711) |
| 1074) | DV_t1_from_Patent_W09322440(A75711)_17mers_2MM.txt | DV t1 from Patent W09322440 |
| | | (A75711) |
| 1075) | DV_t1_from_Patent_W09322440(A75711)_18mers_0MM.txt | DV t1 from Patent W09322440 |
| | | (A75711) |
| 1076) | DV_t1_from_Patent_W09322440(A75711)_18mers_1MM.txt | DV t1 from Patent W09322440 |
| | | (A75711) |
| 1077) | DV_t1_from_Patent_W09322440(A75711)_18mers_2MM.txt | DV t1 from Patent W09322440 |
| | | (A75711) |
| 1078) | DV_t1_genomic_RNA(AB178040)_16mers_0MM.txt | DV t1 genomic RNA (AB178040) |
| 1079) | DV_t1_genomic_RNA(AB178040)_16mers_1MM.txt | DV t1 genomic RNA (AB178040) |
| 1080) | DV_t1_genomic_RNA(AB178040)_16mers_2MM.txt | DV t1 genomic RNA (AB178040) |
| 1081) | DV_t1_genomic_RNA(AB178040)_17mers_0MM.txt | DV t1 genomic RNA (AB178040) |
| 1082) | DV_t1_genomic_RNA(AB178040)_17mers_1MM.txt | DV t1 genomic RNA (AB178040) |
| 1083) | DV_t1_genomic_RNA(AB178040)_17mers_2MM.txt | DV t1 genomic RNA (AB178040) |
| 1084) | DV_t1_genomic_RNA(AB178040)_18mers_0MM.txt | DV t1 genomic RNA (AB178040) |
| 1085) | DV_t1_genomic_RNA(AB178040)_18mers_1MM.txt | DV t1 genomic RNA (AB178040) |
| 1086) | DV_t1_genomic_RNA(AB178040)_18mers_2MM.txt | DV t1 genomic RNA (AB178040) |
| 1087) | DV_t1_isolate_ARG0028(AY277665)_16mers_0MM.txt | DV t1 isolate ARG0028 |
| | | (AY277665) |
| 1088) | DV_t1_isolate_ARG0028(AY277665)_16mers_1MM.txt | DV t1 isolate ARG0028 |
| | | (AY277665) |
| 1089) | DV_t1_isolate_ARG0028(AY277665)_16mers_2MM.txt | DV t1 isolate ARG0028 |
| | | (AY277665) |
| 1090) | DV_t1_isolate_ARG0028(AY277665)_17mers_0MM.txt | DV t1 isolate ARG0028 |
| | | (AY277665) |
| 1091) | DV_t1_isolate_ARG0028(AY277665)_17mers_1MM.txt | DV t1 isolate ARG0028 |
| | | (AY277665) |
| 1092) | DV_t1_isolate_ARG0028(AY277665)_17mers_2MM.txt | DV t1 isolate ARG0028 |
| | | (AY277665) |
| 1093) | DV_t1_isolate_ARG0028(AY277665)_18mers_0MM.txt | DV t1 isolate ARG0028 |
| | | (AY277665) |
| 1094) | DV_t1_isolate_ARG0028(AY277665)_18mers_1MM.txt | DV t1 isolate ARG0028 |
| | | (AY277665) |
| 1095) | DV_t1_isolate_ARG0028(AY277665)_18mers_2MM.txt | DV t1 isolate ARG0028 |
| | | (AY277665) |
| 1096) | DV_t1_isolate_ARG0048(AY277666)_16mers_0MM.txt | DV t1 isolate ARG0048 |
| | | (AY277666) |
| 1097) | DV_t1_isolate_ARG0048(AY277666)_16mers_1MM.txt | DV t1 isolate ARG0048 |
| | | (AY277666) |
| 1098) | DV_t1_isolate_ARG0048(AY277666)_16mers_2MM.txt | DV t1 isolate ARG0048 |
| | | (AY277666) |
| 1099) | DV_t1_isolate_ARG0048(AY277666)_17mers_0MM.txt | DV t1 isolate ARG0048 |
| | | (AY277666) |
| 1100) | DV_t1_isolate_ARG0048(AY277666)_17mers_1MM.txt | DV t1 isolate ARG0048 |
| | | (AY277666) |
| 1101) | DV_t1_isolate_ARG0048(AY277666)_17mers_2MM.txt | DV t1 isolate ARG0048 |
| | | (AY277666) |
| 1102) | DV_t1_isolate_ARG0048(AY277666)_18mers_0MM.txt | DV t1 isolate ARG0048 |
| | | (AY277666) |
| 1103) | DV_t1_isolate_ARG0048(AY277666)_18mers_1MM.txt | DV t1 isolate ARG0048 |
| | | (AY277666) |
| 1104) | DV_t1_isolate_ARG0048(AY277666)_18mers_2MM.txt | DV t1 isolate ARG0048 |
| | | (AY277666) |
| 1105) | DV_t1_isolate_ARG9920(AY277664)_16mers_0MM.txt | DV t1 isolate ARG9920 |
| | | (AY277664) |
| 1106) | DV_t1_isolate_ARG9920(AY277664)_16mers_1MM.txt | DV t1 isolate ARG9920 |
| | | (AY277664) |
| 1107) | DV_t1_isolate_ARG9920(AY277664)_16mers_2MM.txt | DV t1 isolate ARG9920 |
| | | (AY277664) |
| 1108) | DV_t1_isolate_ARG9920(AY277664)_17mers_0MM.txt | DV t1 isolate ARG9920 |
| | | (AY277664) |
| 1109) | DV_t1_isolate_ARG9920(AY277664)_17mers_1MM.txt | DV t1 isolate ARG9920 |
| | | (AY277664) |
| 1110) | DV_t1_isolate_ARG9920(AY277664)_17mers_2MM.txt | DV t1 isolate ARG9920 |
| | | (AY277664) |
| 1111) | DV_t1_isolate_ARG9920(AY277664)_18mers_0MM.txt | DV t1 isolate ARG9920 |
| | | (AY277664) |
| 1112) | DV_t1_isolate_ARG9920(AY277664)_18mers_1MM.txt | DV t1 isolate ARG9920 |
| | | (AY277664) |
| 1113) | DV_t1_isolate_ARG9920(AY277664)_18mers_2MM.txt | DV t1 isolate ARG9920 |
| | | (AY277664) |
| 1114) | DV_t1_isolate_BR-01-MR(AF513110)_16mers_0MM.txt | DV t1 isolate BR-01-MR |
| | | (AF513110) |
| 1115) | DV_t1_isolate_BR-01-MR(AF513110)_16mers_1MM.txt | DV t1 isolate BR-01-MR |
| | | (AF513110) |
| 1116) | DV_t1_isolate_BR-01-MR(AF513110)_16mers_2MM.txt | DV t1 isolate BR-01-MR |
| | | (AF513110) |
| 1117) | DV_t1_isolate_BR-01-MR(AF513110)_17mers_0MM.txt | DV t1 isolate BR-01-MR |
| | | (AF513110) |
| 1118) | DV_t1_isolate_BR-01-MR(AF513110)_17mers_1MM.txt | DV t1 isolate BR-01-MR |
| | | (AF513110) |
| 1119) | DV_t1_isolate_BR-01-MR(AF513110)_17mers_2MM.txt | DV t1 isolate BR-01-MR |
| | | (AF513110) |
| 1120) | DV_t1_isolate_BR-01-MR(AF513110)_18mers_0MM.txt | DV t1 isolate BR-01-MR |
| | | (AF513110) |
| 1121) | DV_t1_isolate_BR-01-MR(AF513110)_18mers_1MM.txt | DV t1 isolate BR-01-MR |
| | | (AF513110) |
| 1122) | DV_t1_isolate_BR-01-MR(AF513110)_18mers_2MM.txt | DV t1 isolate BR-01-MR |
| | | (AF513110) |
| 1123) | DV_t1_recombinant_clone_rDEN1(AY145121)_16mers_0MM.txt | DV t1 recombinant clone |
| | | rDEN1 (AY145121) |
| 1124) | DV_t1_recombinant_clone_rDEN1(AY145121)_16mers_1MM.txt | DV t1 recombinant clone |
| | | rDEN1 (AY145121) |
| 1125) | DV_t1_recombinant_clone_rDEN1(AY145121)_16mers_2MM.txt | DV t1 recombinant clone |
| | | rDEN1 (AY145121) |
| 1126) | DV_t1_recombinant_clone_rDEN1(AY145121)_17mers_0MM.txt | DV t1 recombinant clone |
| | | rDEN1 (AY145121) |
| 1127) | DV_t1_recombinant_clone_rDEN1(AY145121)_17mers_1MM.txt | DV t1 recombinant clone |
| | | rDEN1 (AY145121) |
| 1128) | DV_t1_recombinant_clone_rDEN1(AY145121)_17mers_2MM.txt | DV t1 recombinant clone |
| | | rDEN1 (AY145121) |
| 1129) | DV_t1_recombinant_clone_rDEN1(AY145121)_18mers_0MM.txt | DV t1 recombinant clone |
| | | rDEN1 (AY145121) |
| 1130) | DV_t1_recombinant_clone_rDEN1(AY145121)_18mers_1MM.txt | DV t1 recombinant clone |
| | | rDEN1 (AY145121) |
| 1131) | DV_t1_recombinant_clone_rDEN1(AY145121)_18mers_2MM.txt | DV t1 recombinant clone |
| | | rDEN1 (AY145121) |
| 1132) | DV_t1_recombinant_clone_rDEN1delta30(AY145123)_16mers_0MM.txt | DV t1 |
| | | recombinant clone rDEN1delta30 (AY145123) |
| 1133) | DV_t1_recombinant_clone_rDEN1delta30(AY145123)_16mers_1MM.txt | DV t1 |
| | | recombinant clone rDEN1delta30 (AY145123) |
| 1134) | DV_t1_recombinant_clone_rDEN1delta30(AY145123)_16mers_2MM.txt | DV t1 |
| | | recombinant clone rDEN1delta30 (AY145123) |
| 1135) | DV_t1_recombinant_clone_rDEN1delta30(AY145123)_17mers_0MM.txt | DV t1 |
| | | recombinant clone rDEN1delta30 (AY145123) |
| 1136) | DV_t1_recombinant_clone_rDEN1delta30(AY145123)_17mers_1MM.txt | DV t1 |
| | | recombinant clone rDEN1delta30 (AY145123) |
| 1137) | DV_t1_recombinant_clone_rDEN1delta30(AY145123)_17mers_2MM.txt | DV t1 |
| | | recombinant clone rDEN1delta30 (AY145123) |
| 1138) | DV_t1_recombinant_clone_rDEN1delta30(AY145123)_18mers_0MM.txt | DV t1 |
| | | recombinant clone rDEN1delta30 (AY145123) |
| 1139) | DV_t1_recombinant_clone_rDEN1delta30(AY145123)_18mers_1MM.txt | DV t1 |
| | | recombinant clone rDEN1delta30 (AY145123) |
| 1140) | DV_t1_recombinant_clone_rDEN1delta30(AY145123)_18mers_2MM.txt | DV t1 |
| | | recombinant clone rDEN1delta30 (AY145123) |
| 1141) | DV_t1_recombinant_clone_rDEN1mutF(AY145122)_16mers_0MM.txt | DV t1 recombinant clone |
| | | rDEN1mutF (AY145122) |
| 1142) | DV_t1_recombinant_clone_rDEN1mutF(AY145122)_16mers_1MM.txt | DV t1 recombinant clone |
| | | rDEN1mutF (AY145122) |
| 1143) | DV_t1_recombinant_clone_rDEN1mutF(AY145122)_16mers_2MM.txt | DV t1 recombinant clone |
| | | rDEN1mutF (AY145122) |
| 1144) | DV_t1_recombinant_clone_rDEN1mutF(AY145122)_17mers_0MM.txt | DV t1 recombinant clone |
| | | rDEN1mutF (AY145122) |
| 1145) | DV_t1_recombinant_clone_rDEN1mutF(AY145122)_17mers_1MM.txt | DV t1 recombinant clone |
| | | rDEN1mutF (AY145122) |
| 1146) | DV_t1_recombinant_clone_rDEN1mutF(AY145122)_17mers_2MM.txt | DV t1 recombinant clone |
| | | rDEN1mutF (AY145122) |
| 1147) | DV_t1_recombinant_clone_rDEN1mutF(AY145122)_18mers_0MM.txt | DV t1 recombinant clone |
| | | rDEN1mutF (AY145122) |
| 1148) | DV_t1_recombinant_clone_rDEN1mutF(AY145122)_18mers_1MM.txt | DV t1 recombinant clone |
| | | rDEN1mutF (AY145122) |
| 1149) | DV_t1_recombinant_clone_rDEN1mutF(AY145122)_18mers_2MM.txt | DV t1 recombinant clone |
| | | rDEN1mutF (AY145122) |
| 1150) | DV_t1_strain_16007_(PDK-13)_polyprotein_precursor(AF180818)_16mers_0MM.txt | DV t1 |
| | | strain 16007 (PDK-13) polyprotein precursor (AF180818) |
| 1151) | DV_t1_strain_16007_(PDK-13)_polyprotein_precursor(AF180818)_16mers_1MM.txt | DV t1 |
| | | strain 16007 (PDK-13) polyprotein precursor (AF180818) |
| 1152) | DV_t1_strain_16007_(PDK-13)_polyprotein_precursor(AF180818)_16mers_2MM.txt | DV t1 |
| | | strain 16007 (PDK-13) polyprotein precursor (AF180818) |
| 1153) | DV_t1_strain_16007_(PDK-13)_polyprotein_precursor(AF180818)_17mers_0MM.txt | DV t1 |
| | | strain 16007 (PDK-13) polyprotein precursor (AF180818) |
| 1154) | DV_t1_strain_16007_(PDK-13)_polyprotein_precursor(AF180818)_17mers_1MM.txt | DV t1 |
| | | strain 16007 (PDK-13) polyprotein precursor (AF180818) |
| 1155) | DV_t1_strain_16007_(PDK-13)_polyprotein_precursor(AF180818)_17mers_2MM.txt | DV t1 |
| | | strain 16007 (PDK-13) polyprotein precursor (AF180818) |
| 1156) | DV_t1_strain_16007_(PDK-13)_polyprotein_precursor(AF180818)_18mers_0MM.txt | DV t1 |
| | | strain 16007 (PDK-13) polyprotein precursor (AF180818) |
| 1157) | DV_t1_strain_16007_(PDK-13)_polyprotein_precursor(AF180818)_18mers_1MM.txt | DV t1 |
| | | strain 16007 (PDK-13) polyprotein precursor (AF180818) |
| 1158) | DV_t1_strain_16007_(PDK-13)_polyprotein_precursor(AF180818)_18mers_2MM.txt | DV t1 |
| | | strain 16007 (PDK-13) polyprotein precursor (AF180818) |
| 1159) | DV_t1_strain_16007_polyprotein_precursor(AF180817)_16mers_0MM.txt | DV t1 strain |
| | | 16007 polyprotein precursor (AF180817) |
| 1160) | DV_t1_strain_16007_polyprotein_precursor(AF180817)_16mers_1MM.txt | DV t1 strain |
| | | 16007 polyprotein precursor (AF180817) |
| 1161) | DV_t1_strain_16007_polyprotein_precursor(AF180817)_16mers_2MM.txt | DV t1 strain |
| | | 16007 polyprotein precursor (AF180817) |
| 1162) | DV_t1_strain_16007_polyprotein_precursor(AF180817)_17mers_0MM.txt | DV t1 strain |
| | | 16007 polyprotein precursor (AF180817) |
| 1163) | DV_t1_strain_16007_polyprotein_precursor(AF180817)_17mers_1MM.txt | DV t1 strain |
| | | 16007 polyprotein precursor (AF180817) |
| 1164) | DV_t1_strain_16007_polyprotein_precursor(AF180817)_17mers_2MM.txt | DV t1 strain |
| | | 16007 polyprotein precursor (AF180817) |
| 1165) | DV_t1_strain_16007_polyprotein_precursor(AF180817)_18mers_0MM.txt | DV t1 strain |
| | | 16007 polyprotein precursor (AF180817) |
| 1166) | DV_t1_strain_16007_polyprotein_precursor(AF180817)_18mers_1MM.txt | DV t1 strain |
| | | 16007 polyprotein precursor (AF180817) |
| 1167) | DV_t1_strain_16007_polyprotein_precursor(AF180817)_18mers_2MM.txt | DV t1 strain |
| | | 16007 polyprotein precursor (AF180817) |
| 1168) | DV_t1_strain_259par00_polyprotein_gene(AF514883)_16mers_0MM.txt | DV t1 strain 259par00 |
| | | polyprotein gene (AF514883) |
| 1169) | DV_t1_strain_259par00_polyprotein_gene(AF514883)_16mers_1MM.txt | DV t1 strain 259par00 |
| | | polyprotein gene (AF514883) |
| 1170) | DV_t1_strain_259par00_polyprotein_gene(AF514883)_16mers_2MM.txt | DV t1 strain 259par00 |
| | | polyprotein gene (AF514883) |
| 1171) | DV_t1_strain_259par00_polyprotein_gene(AF514883)_17mers_0MM.txt | DV t1 strain 259par00 |
| | | polyprotein gene (AF514883) |
| 1172) | DV_t1_strain_259par00_polyprotein_gene(AF514883)_17mers_1MM.txt | DV t1 strain 259par00 |
| | | polyprotein gene (AF514883) |
| 1173) | DV_t1_strain_259par00_polyprotein_gene(AF514883)_17mers_2MM.txt | DV t1 strain 259par00 |
| | | polyprotein gene (AF514883) |
| 1174) | DV_t1_strain_259par00_polyprotein_gene(AF514883)_18mers_0MM.txt | DV t1 strain 259par00 |
| | | polyprotein gene (AF514883) |
| 1175) | DV_t1_strain_259par00_polyprotein_gene(AF514883)_18mers_1MM.txt | DV t1 strain 259par00 |
| | | polyprotein gene (AF514883) |
| 1176) | DV_t1_strain_259par00_polyprotein_gene(AF514883)_18mers_2MM.txt | DV t1 strain 259par00 |
| | | polyprotein gene (AF514883) |
| 1177) | DV_t1_strain_280par00_polyprotein_gene(AF514878)_16mers_0MM.txt | DV t1 strain 280par00 |
| | | polyprotein gene (AF514878) |
| 1178) | DV_t1_strain_280par00_polyprotein_gene(AF514878)_16mers_1MM.txt | DV t1 strain 280par00 |
| | | polyprotein gene (AF514878) |
| 1179) | DV_t1_strain_280par00_polyprotein_gene(AF514878)_16mers_2MM.txt | DV t1 strain 280par00 |
| | | polyprotein gene (AF514878) |
| 1180) | DV_t1_strain_280par00_polyprotein_gene(AF514878)_17mers_0MM.txt | DV t1 strain 280par00 |
| | | polyprotein gene (AF514878) |
| 1181) | DV_t1_strain_280par00_polyprotein_gene(AF514878)_17mers_1MM.txt | DV t1 strain 280par00 |
| | | polyprotein gene (AF514878) |
| 1182) | DV_t1_strain_280par00_polyprotein_gene(AF514878)_17mers_2MM.txt | DV t1 strain 280par00 |
| | | polyprotein gene (AF514878) |
| 1183) | DV_t1_strain_280par00_polyprotein_gene(AF514878)_18mers_0MM.txt | DV t1 strain 280par00 |
| | | polyprotein gene (AF514878) |
| 1184) | DV_t1_strain_280par00_polyprotein_gene(AF514878)_18mers_1MM.txt | DV t1 strain 280par00 |
| | | polyprotein gene (AF514878) |
| 1185) | DV_t1_strain_280par00_polyprotein_gene(AF514878)_18mers_2MM.txt | DV t1 strain 280par00 |
| | | polyprotein gene (AF514878) |
| 1186) | Dv_t1_strain_293arg00_polyprotein_gene(AY206457)_16mers_0MM.txt | Dv t1 strain 293arg00 |
| | | polyprotein gene (AY206457) |
| 1187) | Dv_t1_strain_293arg00_polyprotein_gene(AY206457)_16mers_1MM.txt | Dv t1 strain 293arg00 |
| | | polyprotein gene (AY206457) |
| 1188) | Dv_t1_strain_293arg00_polyprotein_gene(AY206457)_16mers_2MM.txt | Dv t1 strain 293arg00 |
| | | polyprotein gene (AY206457) |
| 1189) | Dv_t1_strain_293arg00_polyprotein_gene(AY206457)_17mers_0MM.txt | Dv t1 strain 293arg00 |
| | | polyprotein gene (AY206457) |
| 1190) | Dv_t1_strain_293arg00_polyprotein_gene(AY206457)_17mers_1MM.txt | Dv t1 strain 293arg00 |
| | | polyprotein gene (AY206457) |
| 1191) | Dv_t1_strain_293arg00_polyprotein_gene(AY206457)_17mers_2MM.txt | Dv t1 strain 293arg00 |
| | | polyprotein gene (AY206457) |
| 1192) | Dv_t1_strain_293arg00_polyprotein_gene(AY206457)_18mers_0MM.txt | Dv t1 strain 293arg00 |
| | | polyprotein gene (AY206457) |
| 1193) | Dv_t1_strain_293arg00_polyprotein_gene(AY206457)_18mers_1MM.txt | Dv t1 strain 293arg00 |
| | | polyprotein gene (AY206457) |
| 1194) | Dv_t1_strain_293arg00_polyprotein_gene(AY206457)_18mers_2MM.txt | Dv t1 strain 293arg00 |
| | | polyprotein gene (AY206457) |
| 1195) | DV_t1_strain_295arg00_polyprotein_gene(AF514885)_16mers_0MM.txt | DV t1 strain 295arg00 |
| | | polyprotein gene (AF514885) |
| 1196) | DV_t1_strain_295arg00_polyprotein_gene(AF514885)_16mers_1MM.txt | DV t1 strain 295arg00 |
| | | polyprotein gene (AF514885) |
| 1197) | DV_t1_strain_295arg00_polyprotein_gene(AF514885)_16mers_2MM.txt | DV t1 strain 295arg00 |
| | | polyprotein gene (AF514885) |
| 1198) | DV_t1_strain_295arg00_polyprotein_gene(AF514885)_17mers_0MM.txt | DV t1 strain 295arg00 |
| | | polyprotein gene (AF514885) |
| 1199) | DV_t1_strain_295arg00_polyprotein_gene(AF514885)_17mers_1MM.txt | DV t1 strain 295arg00 |
| | | polyprotein gene (AF514885) |
| 1200) | DV_t1_strain_295arg00_polyprotein_gene(AF514885)_17mers_2MM.txt | DV t1 strain 295arg00 |
| | | polyprotein gene (AF514885) |
| 1201) | DV_t1_strain_295arg00_polyprotein_gene(AF514885)_18mers_0MM.txt | DV t1 strain 295arg00 |
| | | polyprotein gene (AF514885) |
| 1202) | DV_t1_strain_295arg00_polyprotein_gene(AF514885)_18mers_1MM.txt | DV t1 strain 295arg00 |
| | | polyprotein gene (AF514885) |
| 1203) | DV_t1_strain_295arg00_polyprotein_gene(AF514885)_18mers_2MM.txt | DV t1 strain 295arg00 |
| | | polyprotein gene (AF514885) |
| 1204) | DV_t1_strain_297arg00_envelope_glycoprotein_gene(AF514889)_16mers_0MM.txt | DV t1 |
| | | strain 297arg00 envelope glycoprotein gene (AF514889) |
| 1205) | DV_t1_strain_297arg00_envelope_glycoprotein_gene(AF514889)_16mers_1MM.txt | DV t1 |
| | | strain 297arg00 envelope glycoprotein gene (AF514889) |
| 1206) | DV_t1_strain_297arg00_envelope_glycoprotein_gene(AF514889)_16mers_2MM.txt | DV t1 |
| | | strain 297arg00 envelope glycoprotein gene (AF514889) |
| 1207) | DV_t1_strain_297arg00_envelope_glycoprotein_gene(AF514889)_17mers_0MM.txt | DV t1 |
| | | strain 297arg00 envelope glycoprotein gene (AF514889) |
| 1208) | DV_t1_strain_297arg00_envelope_glycoprotein_gene(AF514889)_17mers_1MM.txt | DV t1 |
| | | strain 297arg00 envelope glycoprotein gene (AF514889) |
| 1209) | DV_t1_strain_297arg00_envelope_glycoprotein_gene(AF514889)_17mers_2MM.txt | DV t1 |
| | | strain 297arg00 envelope glycoprotein gene (AF514889) |
| 1210) | DV_t1_strain_297arg00_envelope_glycoprotein_gene(AF514889)_18mers_0MM.txt | DV t1 |
| | | strain 297arg00 envelope glycoprotein gene (AF514889) |
| 1211) | DV_t1_strain_297arg00_envelope_glycoprotein_gene(AF514889)_18mers_1MM.txt | DV t1 |
| | | strain 297arg00 envelope glycoprotein gene (AF514889) |
| 1212) | DV_t1_strain_297arg00_envelope_glycoprotein_gene(AF514889)_18mers_2MM.txt | DV t1 |
| | | strain 297arg00 envelope glycoprotein gene (AF514889) |
| 1213) | DV_t1_strain_301arg00_polyprotein_gene(AF514876)_16mers_0MM.txt | DV t1 strain 301arg00 |
| | | polyprotein gene (AF514876) |
| 1214) | DV_t1_strain_301arg00_polyprotein_gene(AF514876)_16mers_1MM.txt | DV t1 strain 301arg00 |
| | | polyprotein gene (AF514876) |
| 1215) | DV_t1_strain_301arg00_polyprotein_gene(AF514876)_16mers_2MM.txt | DV t1 strain 301arg00 |
| | | polyprotein gene (AF514876) |
| 1216) | DV_t1_strain_301arg00_polyprotein_gene(AF514876)_17mers_0MM.txt | DV t1 strain 301arg00 |
| | | polyprotein gene (AF514876) |
| 1217) | DV_t1_strain_301arg00_polyprotein_gene(AF514876)_17mers_1MM.txt | DV t1 strain 301arg00 |
| | | polyprotein gene (AF514876) |
| 1218) | DV_t1_strain_301arg00_polyprotein_gene(AF514876)_17mers_2MM.txt | DV t1 strain 301arg00 |
| | | polyprotein gene (AF514876) |
| 1219) | DV_t1_strain_301arg00_polyprotein_gene(AF514876)_18mers_0MM.txt | DV t1 strain 301arg00 |
| | | polyprotein gene (AF514876) |
| 1220) | DV_t1_strain_301arg00_polyprotein_gene(AF514876)_18mers_1MM.txt | DV t1 strain 301arg00 |
| | | polyprotein gene (AF514876) |
| 1221) | DV_t1_strain_301arg00_polyprotein_gene(AF514876)_18mers_2MM.txt | DV t1 strain 301arg00 |
| | | polyprotein gene (AF514876) |
| 1222) | DV_t1_strain_A88(AB074761)_16mers_0MM.txt | DV t1 strain A88 (AB074761) |
| 1223) | DV_t1_strain_A88(AB074761)_16mers_1MM.txt | DV t1 strain A88 (AB074761) |
| 1224) | DV_t1_strain_A88(AB074761)_16mers_2MM.txt | DV t1 strain A88 (AB074761) |
| 1225) | DV_t1_strain_A88(AB074761)_17mers_0MM.txt | DV t1 strain A88 (AB074761) |
| 1226) | DV_t1_strain_A88(AB074761)_17mers_1MM.txt | DV t1 strain A88 (AB074761) |
| 1227) | DV_t1_strain_A88(AB074761)_17mers_2MM.txt | DV t1 strain A88 (AB074761) |
| 1228) | DV_t1_strain_A88(AB074761)_18mers_0MM.txt | DV t1 strain A88 (AB074761) |
| 1229) | DV_t1_strain_A88(AB074761)_18mers_1MM.txt | DV t1 strain A88 (AB074761) |
| 1230) | DV_t1_strain_A88(AB074761)_18mers_2MM.txt | DV t1 strain A88 (AB074761) |
| 1231) | DV_t1_strain_BR-97-111(AF311956)_16mers_0MM.txt | DV t1 strain BR-97-111 |
| | | (AF311956) |
| 1232) | DV_t1_strain_BR-97-111(AF311956)_16mers_1MM.txt | DV t1 strain BR-97-111 |
| | | (AF311956) |
| 1233) | DV_t1_strain_BR-97-111(AF311956)_16mers_2MM.txt | DV t1 strain BR-97-111 |
| | | (AF311956) |
| 1234) | DV_t1_strain_BR-97-111(AF311956)_17mers_0MM.txt | DV t1 strain BR-97-111 |
| | | (AF311956) |
| 1235) | DV_t1_strain_BR-97-111(AF311956)_17mers_1MM.txt | DV t1 strain BR-97-111 |
| | | (AF311956) |
| 1236) | DV_t1_strain_BR-97-111(AF311956)_17mers_2MM.txt | DV t1 strain BR-97-111 |
| | | (AF311956) |
| 1237) | DV_t1_strain_BR-97-111(AF311956)_18mers_0MM.txt | DV t1 strain BR-97-111 |
| | | (AF311956) |
| 1238) | DV_t1_strain_BR-97-111(AF311956)_18mers_1MM.txt | DV t1 strain BR-97-111 |
| | | (AF311956) |
| 1239) | DV_t1_strain_BR-97-111(AF311956)_18mers_2MM.txt | DV t1 strain BR-97-111 |
| | | (AF311956) |
| 1240) | DV_t1_strain_BR-97-233(AF311958)_16mers_0MM.txt | DV t1 strain BR-97-233 |
| | | (AF311958) |
| 1241) | DV_t1_strain_BR-97-233(AF311958)_16mers_1MM.txt | DV t1 strain BR-97-233 |
| | | (AF311958) |
| 1242) | DV_t1_strain_BR-97-233(AF311958)_16mers_2MM.txt | DV t1 strain BR-97-233 |
| | | (AF311958) |
| 1243) | DV_t1_strain_BR-97-233(AF311958)_17mers_0MM.txt | DV t1 strain BR-97-233 |
| | | (AF311958) |
| 1244) | DV_t1_strain_BR-97-233(AF311958)_17mers_1MM.txt | DV t1 strain BR-97-233 |
| | | (AF311958) |
| 1245) | DV_t1_strain_BR-97-233(AF311958)_17mers_2MM.txt | DV t1 strain BR-97-233 |
| | | (AF311958) |
| 1246) | DV_t1_strain_BR-97-233(AF311958)_18mers_0MM.txt | DV t1 strain BR-97-233 |
| | | (AF311958) |
| 1247) | DV_t1_strain_BR-97-233(AF311958)_18mers_1MM.txt | DV t1 strain BR-97-233 |
| | | (AF311958) |
| 1248) | DV_t1_strain_BR-97-233(AF311958)_18mers_2MM.txt | DV t1 strain BR-97-233 |
| | | (AF311958) |
| 1249) | DV_t1_strain_BR-97-409(AF311957)_16mers_0MM.txt | DV t1 strain BR-97-409 |
| | | (AF311957) |
| 1250) | DV_t1_strain_BR-97-409(AF311957)_16mers_1MM.txt | DV t1 strain BR-97-409 |
| | | (AF311957) |
| 1251) | DV_t1_strain_BR-97-409(AF311957)_16mers_2MM.txt | DV t1 strain BR-97-409 |
| | | (AF311957) |
| 1252) | DV_t1_strain_BR-97-409(AF311957)_17mers_0MM.txt | DV t1 strain BR-97-409 |
| | | (AF311957) |
| 1253) | DV_t1_strain_BR-97-409(AF311957)_17mers_1MM.txt | DV t1 strain BR-97-409 |
| | | (AF311957) |
| 1254) | DV_t1_strain_BR-97-409(AF311957)_17mers_2MM.txt | DV t1 strain BR-97-409 |
| | | (AF311957) |
| 1255) | DV_t1_strain_BR-97-409(AF311957)_18mers_0MM.txt | DV t1 strain BR-97-409 |
| | | (AF311957) |
| 1256) | DV_t1_strain_BR-97-409(AF311957)_18mers_1MM.txt | DV t1 strain BR-97-409 |
| | | (AF311957) |
| 1257) | DV_t1_strain_BR-97-409(AF311957)_18mers_2MM.txt | DV t1 strain BR-97-409 |
| | | (AF311957) |
| 1258) | DV_t1_strain_Den1BR-90(AF226685)_16mers_0MM.txt | DV t1 strain Den1BR-90 |
| | | (AF226685) |
| 1259) | DV_t1_strain_Den1BR-90(AF226685)_16mers_1MM.txt | DV t1 strain Den1BR-90 |
| | | (AF226685) |
| 1260) | DV_t1_strain_Den1BR-90(AF226685)_16mers_2MM.txt | DV t1 strain Den1BR-90 |
| | | (AF226685) |
| 1261) | DV_t1_strain_Den1BR-90(AF226685)_17mers_0MM.txt | DV t1 strain Den1BR-90 |
| | | (AF226685) |
| 1262) | DV_t1_strain_Den1BR-90(AF226685)_17mers_1MM.txt | DV t1 strain Den1BR-90 |
| | | (AF226685) |
| 1263) | DV_t1_strain_Den1BR-90(AF226685)_17mers_2MM.txt | DV t1 strain Den1BR-90 |
| | | (AF226685) |
| 1264) | DV_t1_strain_Den1BR-90(AF226685)_18mers_0MM.txt | DV t1 strain Den1BR-90 |
| | | (AF226685) |
| 1265) | DV_t1_strain_Den1BR-90(AF226685)_18mers_1MM.txt | DV t1 strain Den1BR-90 |
| | | (AF226685) |
| 1266) | DV_t1_strain_Den1BR-90(AF226685)_18mers_2MM.txt | DV t1 strain Den1BR-90 |
| | | (AF226685) |
| 1267) | DV_t1_strain_FGA-89(AF226687)_16mers_0MM.txt | DV t1 strain FGA-89 (AF226687) |
| 1268) | DV_t1_strain_FGA-89(AF226687)_16mers_1MM.txt | DV t1 strain FGA-89 (AF226687) |
| 1269) | DV_t1_strain_FGA-89(AF226687)_16mers_2MM.txt | DV t1 strain FGA-89 (AF226687) |
| 1270) | DV_t1_strain_FGA-89(AF226687)_17mers_0MM.txt | DV t1 strain FGA-89 (AF226687) |
| 1271) | DV_t1_strain_FGA-89(AF226687)_17mers_1MM.txt | DV t1 strain FGA-89 (AF226687) |
| 1272) | DV_t1_strain_FGA-89(AF226687)_17mers_2MM.txt | DV t1 strain FGA-89 (AF226687) |
| 1273) | DV_t1_strain_FGA-89(AF226687)_18mers_0MM.txt | DV t1 strain FGA-89 (AF226687) |
| 1274) | DV_t1_strain_FGA-89(AF226687)_18mers_1MM.txt | DV t1 strain FGA-89 (AF226687) |
| 1275) | DV_t1_strain_FGA-89(AF226687)_18mers_2MM.txt | DV t1 strain FGA-89 (AF226687) |
| 1276) | DV_t1_strain_FGA-NA_d1d(AF226686)_16mers_0MM.txt | DV t1 strain FGA-NA d1d |
| | | (AF226686) |
| 1277) | DV_t1_strain_FGA-NA_d1d(AF226686)_16mers_1MM.txt | DV t1 strain FGA-NA d1d |
| | | (AF226686) |
| 1278) | DV_t1_strain_FGA-NA_d1d(AF226686)_16mers_2MM.txt | DV t1 strain FGA-NA d1d |
| | | (AF226686) |
| 1279) | DV_t1_strain_FGA-NA_d1d(AF226686)_17mers_0MM.txt | DV t1 strain FGA-NA d1d |
| | | (AF226686) |
| 1280) | DV_t1_strain_FGA-NA_d1d(AF226686)_17mers_1MM.txt | DV t1 strain FGA-NA d1d |
| | | (AF226686) |
| 1281) | DV_t1_strain_FGA-NA_d1d(AF226686)_17mers_2MM.txt | DV t1 strain FGA-NA d1d |
| | | (AF226686) |
| 1282) | DV_t1_strain_FGA-NA_d1d(AF226686)_18mers_0MM.txt | DV t1 strain FGA-NA d1d |
| | | (AF226686) |
| 1283) | DV_t1_strain_FGA-NA_d1d(AF226686)_18mers_1MM.txt | DV t1 strain FGA-NA d1d |
| | | (AF226686) |
| 1284) | DV_t1_strain_FGA-NA_d1d(AF226686)_18mers_2MM.txt | DV t1 strain FGA-NA d1d |
| | | (AF226686) |
| 1285) | DV_t1_strain_GZ-80_polyprotein_precursor(AF350498)_16mers_0MM.txt | DV t1 strain GZ- |
| | | 80 polyprotein precursor (AF350498) |
| 1286) | DV_t1_strain_GZ-80_polyprotein_precursor(AF350498)_16mers_1MM.txt | DV t1 strain GZ- |
| | | 80 polyprotein precursor (AF350498) |
| 1287) | DV_t1_strain_GZ-80_polyprotein_precursor(AF350498)_16mers_2MM.txt | DV t1 strain GZ- |
| | | 80 polyprotein precursor (AF350498) |
| 1288) | DV_t1_strain_GZ-80_polyprotein_precursor(AF350498)_17mers_0MM.txt | DV t1 strain GZ- |
| | | 80 polyprotein precursor (AF350498) |
| 1289) | DV_t1_strain_GZ-80_polyprotein_precursor(AF350498)_17mers_1MM.txt | DV t1 strain GZ- |
| | | 80 polyprotein precursor (AF350498) |
| 1290) | DV_t1_strain_GZ-80_polyprotein_precursor(AF350498)_17mers_2MM.txt | DV t1 strain GZ- |
| | | 80 polyprotein precursor (AF350498) |
| 1291) | DV_t1_strain_GZ-80_polyprotein_precursor(AF350498)_18mers_0MM.txt | DV t1 strain GZ- |
| | | 80 polyprotein precursor (AF350498) |
| 1292) | DV_t1_strain_GZ-80_polyprotein_precursor(AF350498)_18mers_1MM.txt | DV t1 strain GZ- |
| | | 80 polyprotein precursor (AF350498) |
| 1293) | DV_t1_strain_GZ-80_polyprotein_precursor(AF350498)_18mers_2MM.txt | DV t1 strain GZ- |
| | | 80 polyprotein precursor (AF350498) |
| 1294) | DV_t1_strain_Mochizuki(AB074760)_16mers_0MM.txt | DV t1 strain Mochizuki |
| | | (AB074760) |
| 1295) | DV_t1_strain_Mochizuki(AB074760)_16mers_1MM.txt | DV t1 strain Mochizuki |
| | | (AB074760) |
| 1296) | DV_t1_strain_Mochizuki(AB074760)_16mers_2MM.txt | DV t1 strain Mochizuki |
| | | (AB074760) |
| 1297) | DV_t1_strain_Mochizuki(AB074760)_17mers_0MM.txt | DV t1 strain Mochizuki |
| | | (AB074760) |
| 1298) | DV_t1_strain_Mochizuki(AB074760)_17mers_1MM.txt | DV t1 strain Mochizuki |
| | | (AB074760) |
| 1299) | DV_t1_strain_Mochizuki(AB074760)_17mers_2MM.txt | DV t1 strain Mochizuki |
| | | (AB074760) |
| 1300) | DV_t1_strain_Mochizuki(AB074760)_18mers_0MM.txt | DV t1 strain Mochizuki |
| | | (AB074760) |
| 1301) | DV_t1_strain_Mochizuki(AB074760)_18mers_1MM.txt | DV t1 strain Mochizuki |
| | | (AB074760) |
| 1302) | DV_t1_strain_Mochizuki(AB074760)_18mers_2MM.txt | DV t1 strain Mochizuki |
| | | (AB074760) |
| 1303) | DV_t2(M29095)_16mers_0MM.txt | DV t2 (M29095) |
| 1304) | DV_t2(M29095)_16mers_1MM.txt | DV t2 (M29095) |
| 1305) | DV_t2(M29095)_16mers_2MM.txt | DV t2 (M29095) |
| 1306) | DV_t2(M29095)_17mers_0MM.txt | DV t2 (M29095) |
| 1307) | DV_t2(M29095)_17mers_1MM.txt | DV t2 (M29095) |
| 1308) | DV_t2(M29095)_17mers_2MM.txt | DV t2 (M29095) |
| 1309) | DV_t2(M29095)_18mers_0MM.txt | DV t2 (M29095) |
| 1310) | DV_t2(M29095)_18mers_1MM.txt | DV t2 (M29095) |
| 1311) | DV_t2(M29095)_18mers_2MM.txt | DV t2 (M29095) |
| 1312) | DV_t2_China_isolate_04(AF119661)_16mers_0MM.txt | DV t2 China isolate 04 |
| | | (AF119661) |
| 1313) | DV_t2_China_isolate_04(AF119661)_16mers_1MM.txt | DV t2 China isolate 04 |
| | | (AF119661) |
| 1314) | DV_t2_China_isolate_04(AF119661)_16mers_2MM.txt | DV t2 China isolate 04 |
| | | (AF119661) |
| 1315) | DV_t2_China_isolate_04(AF119661)_17mers_0MM.txt | DV t2 China isolate 04 |
| | | (AF119661) |
| 1316) | DV_t2_China_isolate_04(AF119661)_17mers_1MM.txt | DV t2 China isolate 04 |
| | | (AF119661) |
| 1317) | DV_t2_China_isolate_04(AF119661)_17mers_2MM.txt | DV t2 China isolate 04 |
| | | (AF119661) |
| 1318) | DV_t2_China_isolate_04(AF119661)_18mers_0MM.txt | DV t2 China isolate 04 |
| | | (AF119661) |
| 1319) | DV_t2_China_isolate_04(AF119661)_18mers_1MM.txt | DV t2 China isolate 04 |
| | | (AF119661) |
| 1320) | DV_t2_China_isolate_04(AF119661)_18mers_2MM.txt | DV t2 China isolate 04 |
| | | (AF119661) |
| 1321) | DV_t2_Jamaica(M20558)_16mers_0MM.txt | DV t2 Jamaica (M20558) |
| 1322) | DV_t2_Jamaica(M20558)_16mers_1MM.txt | DV t2 Jamaica (M20558) |
| 1323) | DV_t2_Jamaica(M20558)_16mers_2MM.txt | DV t2 Jamaica (M20558) |
| 1324) | DV_t2_Jamaica(M20558)_17mers_0MM.txt | DV t2 Jamaica (M20558) |
| 1325) | DV_t2_Jamaica(M20558)_17mers_1MM.txt | DV t2 Jamaica (M20558) |
| 1326) | DV_t2_Jamaica(M20558)_17mers_2MM.txt | DV t2 Jamaica (M20558) |
| 1327) | DV_t2_Jamaica(M20558)_18mers_0MM.txt | DV t2 Jamaica (M20558) |
| 1328) | DV_t2_Jamaica(M20558)_18mers_1MM.txt | DV t2 Jamaica (M20558) |
| 1329) | DV_t2_Jamaica(M20558)_18mers_2MM.txt | DV t2 Jamaica (M20558) |
| 1330) | DV_t2_polyprotein_mRNA(U87412)_16mers_0MM.txt | DV t2 polyprotein mRNA |
| | | (U87412) |
| 1331) | DV_t2_polyprotein_mRNA(U87412)_16mers_1MM.txt | DV t2 polyprotein mRNA |
| | | (U87412) |
| 1332) | DV_t2_polyprotein_mRNA(U87412)_16mers_2MM.txt | DV t2 polyprotein mRNA |
| | | (U87412) |
| 1333) | DV_t2_polyprotein_mRNA(U87412)_17mers_0MM.txt | DV t2 polyprotein mRNA |
| | | (U87412) |
| 1334) | DV_t2_polyprotein_mRNA(U87412)_17mers_1MM.txt | DV t2 polyprotein mRNA |
| | | (U87412) |
| 1335) | DV_t2_polyprotein_mRNA(U87412)_17mers_2MM.txt | DV t2 polyprotein mRNA |
| | | (U87412) |
| 1336) | DV_t2_polyprotein_mRNA(U87412)_18mers_0MM.txt | DV t2 polyprotein mRNA |
| | | (U87412) |
| 1337) | DV_t2_polyprotein_mRNA(U87412)_18mers_1MM.txt | DV t2 polyprotein mRNA |
| | | (U87412) |
| 1338) | DV_t2_polyprotein_mRNA(U87412)_18mers_2MM.txt | DV t2 polyprotein mRNA |
| | | (U87412) |
| 1339) | DV_t2_strain_131_polyprotein_gene(AF100469)_16mers_0MM.txt | DV t2 strain 131 |
| | | polyprotein gene (AF100469) |
| 1340) | DV_t2_strain_131_polyprotein_gene(AF100469)_16mers_1MM.txt | DV t2 strain 131 |
| | | polyprotein gene (AF100469) |
| 1341) | DV_t2_strain_131_polyprotein_gene(AF100469)_16mers_2MM.txt | DV t2 strain 131 |
| | | polyprotein gene (AF100469) |
| 1342) | DV_t2_strain_131_polyprotein_gene(AF100469)_17mers_0MM.txt | DV t2 strain 131 |
| | | polyprotein gene (AF100469) |
| 1343) | DV_t2_strain_131_polyprotein_gene(AF100469)_17mers_1MM.txt | DV t2 strain 131 |
| | | polyprotein gene (AF100469) |
| 1344) | DV_t2_strain_131_polyprotein_gene(AF100469)_17mers_2MM.txt | DV t2 strain 131 |
| | | polyprotein gene (AF100469) |
| 1345) | DV_t2_strain_131_polyprotein_gene(AF100469)_18mers_0MM.txt | DV t2 strain 131 |
| | | polyprotein gene (AF100469) |
| 1346) | DV_t2_strain_131_polyprotein_gene(AF100469)_18mers_1MM.txt | DV t2 strain 131 |
| | | polyprotein gene (AF100469) |
| 1347) | DV_t2_strain_131_polyprotein_gene(AF100469)_18mers_2MM.txt | DV t2 strain 131 |
| | | polyprotein gene (AF100469) |
| 1348) | DV_t2_strain_16681_capsid_protein(C)_gene(M84727)_16mers_0MM.txt | DV t2 strain |
| | | 16681 capsid protein (C) gene (M84727) |
| 1349) | DV_t2_strain_16681_capsid_protein(C)_gene(M84727)_16mers_1MM.txt | DV t2 strain |
| | | 16681 capsid protein (C) gene (M84727) |
| 1350) | DV_t2_strain_16681_capsid_protein(C)_gene(M84727)_16mers_2MM.txt | DV t2 strain |
| | | 16681 capsid protein (C) gene (M84727) |
| 1351) | DV_t2_strain_16681_capsid_protein(C)_gene(M84727)_17mers_0MM.txt | DV t2 strain |
| | | 16681 capsid protein (C) gene (M84727) |
| 1352) | DV_t2_strain_16681_capsid_protein(C)_gene(M84727)_17mers_1MM.txt | DV t2 strain |
| | | 16681 capsid protein (C) gene (M84727) |
| 1353) | DV_t2_strain_16681_capsid_protein(C)_gene(M84727)_17mers_2MM.txt | DV t2 strain |
| | | 16681 capsid protein (C) gene (M84727) |
| 1354) | DV_t2_strain_16681_capsid_protein(C)_gene(M84727)_18mers_0MM.txt | DV t2 strain |
| | | 16681 capsid protein (C) gene (M84727) |
| 1355) | DV_t2_strain_16681_capsid_protein(C)_gene(M84727)_18mers_1MM.txt | DV t2 strain |
| | | 16681 capsid protein (C) gene (M84727) |
| 1356) | DV_t2_strain_16681_capsid_protein(C)_gene(M84727)_18mers_2MM.txt | DV t2 strain |
| | | 16681 capsid protein (C) gene (M84727) |
| 1357) | DV_t2_strain_16681_polyprotein_mRNA(U87411)_16mers_0MM.txt | DV t2 strain 16681 |
| | | polyprotein mRNA (U87411) |
| 1358) | DV_t2_strain_16681_polyprotein_mRNA(U87411)_16mers_1MM.txt | DV t2 strain 16681 |
| | | polyprotein mRNA (U87411) |
| 1359) | DV_t2_strain_16681_polyprotein_mRNA(U87411)_16mers_2MM.txt | DV t2 strain 16681 |
| | | polyprotein mRNA (U87411) |
| 1360) | DV_t2_strain_16681_polyprotein_mRNA(U87411)_17mers_0MM.txt | DV t2 strain 16681 |
| | | polyprotein mRNA (U87411) |
| 1361) | DV_t2_strain_16681_polyprotein_mRNA(U87411)_17mers_1MM.txt | DV t2 strain 16681 |
| | | polyprotein mRNA (U87411) |
| 1362) | DV_t2_strain_16681_polyprotein_mRNA(U87411)_17mers_2MM.txt | DV t2 strain 16681 |
| | | polyprotein mRNA (U87411) |
| 1363) | DV_t2_strain_16681_polyprotein_mRNA(U87411)_18mers_0MM.txt | DV t2 strain 16681 |
| | | polyprotein mRNA (U87411) |
| 1364) | DV_t2_strain_16681_polyprotein_mRNA(U87411)_18mers_1MM.txt | DV t2 strain 16681 |
| | | polyprotein mRNA (U87411) |
| 1365) | DV_t2_strain_16681_polyprotein_mRNA(U87411)_18mers_2MM.txt | DV t2 strain 16681 |
| | | polyprotein mRNA (U87411) |
| 1366) | DV_t2_strain_16681-PDK53_caspid_protein(C)_gene(M84728)_16mers_0MM.txt | DV t2 |
| | | strain 16681-PDK53 caspid protein (C) gene (M84728) |
| 1367) | DV_t2_strain_16681-PDK53_caspid_protein(C)_gene(M84728)_16mers_1MM.txt | DV t2 |
| | | strain 16681-PDK53 caspid protein (C) gene (M84728) |
| 1368) | DV_t2_strain_16681-PDK53_caspid_protein(C)_gene(M84728)_16mers_2MM.txt | DV t2 |
| | | strain 16681-PDK53 caspid protein (C) gene (M84728) |
| 1369) | DV_t2_strain_16681-PDK53_caspid_protein(C)_gene(M84728)_17mers_0MM.txt | DV t2 |
| | | strain 16681-PDK53 caspid protein (C) gene (M84728) |
| 1370) | DV_t2_strain_16681-PDK53_caspid_protein(C)_gene(M84728)_17mers_1MM.txt | DV t2 |
| | | strain 16681-PDK53 caspid protein (C) gene (M84728) |
| 1371) | DV_t2_strain_16681-PDK53_caspid_protein(C)_gene(M84728)_17mers_2MM.txt | DV t2 |
| | | strain 16681-PDK53 caspid protein (C) gene (M84728) |
| 1372) | DV_t2_strain_16681-PDK53_caspid_protein(C)_gene(M84728)_18mers_0MM.txt | DV t2 |
| | | strain 16681-PDK53 caspid protein (C) gene (M84728) |
| 1373) | DV_t2_strain_16681-PDK53_caspid_protein(C)_gene(M84728)_18mers_1MM.txt | DV t2 |
| | | strain 16681-PDK53 caspid protein (C) gene (M84728) |
| 1374) | DV_t2_strain_16681-PDK53_caspid_protein(C)_gene(M84728)_18mers_2MM.txt | DV t2 |
| | | strain 16681-PDK53 caspid protein (C) gene (M84728) |
| 1375) | DV_t2_strain_43(AF204178)_16mers_0MM.txt | DV t2 strain 43 (AF204178) |
| 1376) | DV_t2_strain_43(AF204178)_16mers_1MM.txt | DV t2 strain 43 (AF204178) |
| 1377) | DV_t2_strain_43(AF204178)_16mers_2MM.txt | DV t2 strain 43 (AF204178) |
| 1378) | DV_t2_strain_43(AF204178)_17mers_0MM.txt | DV t2 strain 43 (AF204178) |
| 1379) | DV_t2_strain_43(AF204178)_17mers_1MM.txt | DV t2 strain 43 (AF204178) |
| 1380) | DV_t2_strain_43(AF204178)_17mers_2MM.txt | DV t2 strain 43 (AF204178) |
| 1381) | DV_t2_strain_43(AF204178)_18mers_0MM.txt | DV t2 strain 43 (AF204178) |
| 1382) | DV_t2_strain_43(AF204178)_18mers_1MM.txt | DV t2 strain 43 (AF204178) |
| 1383) | DV_t2_strain_43(AF204178)_18mers_2MM.txt | DV t2 strain 43 (AF204178) |
| 1384) | DV_t2_strain_44(AF204177)_16mers_0MM.txt | DV t2 strain 44 (AF204177) |
| 1385) | DV_t2_strain_44(AF204177)_16mers_1MM.txt | DV t2 strain 44 (AF204177) |
| 1386) | DV_t2_strain_44(AF204177)_16mers_2MM.txt | DV t2 strain 44 (AF204177) |
| 1387) | DV_t2_strain_44(AF204177)_17mers_0MM.txt | DV t2 strain 44 (AF204177) |
| 1388) | DV_t2_strain_44(AF204177)_17mers_1MM.txt | DV t2 strain 44 (AF204177) |
| 1389) | DV_t2_strain_44(AF204177)_17mers_2MM.txt | DV t2 strain 44 (AF204177) |
| 1390) | DV_t2_strain_44(AF204177)_18mers_0MM.txt | DV t2 strain 44 (AF204177) |
| 1391) | DV_t2_strain_44(AF204177)_18mers_1MM.txt | DV t2 strain 44 (AF204177) |
| 1392) | DV_t2_strain_44(AF204177)_18mers_2MM.txt | DV t2 strain 44 (AF204177) |
| 1393) | DV_t2_strain_BR64022(AF489932)_16mers_0MM.txt | DV t2 strain BR64022 (AF489932) |
| 1394) | DV_t2_strain_BR64022(AF489932)_16mers_1MM.txt | DV t2 strain BR64022 (AF489932) |
| 1395) | DV_t2_strain_BR64022(AF489932)_16mers_2MM.txt | DV t2 strain BR64022 (AF489932) |
| 1396) | DV_t2_strain_BR64022(AF489932)_17mers_0MM.txt | DV t2 strain BR64022 (AF489932) |
| 1397) | DV_t2_strain_BR64022(AF489932)_17mers_1MM.txt | DV t2 strain BR64022 (AF489932) |
| 1398) | DV_t2_strain_BR64022(AF489932)_17mers_2MM.txt | DV t2 strain BR64022 (AF489932) |
| 1399) | DV_t2_strain_BR64022(AF489932)_18mers_0MM.txt | DV t2 strain BR64022 (AF489932) |
| 1400) | DV_t2_strain_BR64022(AF489932)_18mers_1MM.txt | DV t2 strain BR64022 (AF489932) |
| 1401) | DV_t2_strain_BR64022(AF489932)_18mers_2MM.txt | DV t2 strain BR64022 (AF489932) |
| 1402) | DV_t2_strain_C0166_polyprotein_gene(AF100463)_16mers_0MM.txt | DV t2 strain C0166 |
| | | polyprotein gene (AF100463) |
| 1403) | DV_t2_strain_C0166_polyprotein_gene(AF100463)_16mers_1MM.txt | DV t2 strain C0166 |
| | | polyprotein gene (AF100463) |
| 1404) | DV_t2_strain_C0166_polyprotein_gene(AF100463)_16mers_2MM.txt | DV t2 strain C0166 |
| | | polyprotein gene (AF100463) |
| 1405) | DV_t2_strain_C0166_polyprotein_gene(AF100463)_17mers_0MM.txt | DV t2 strain C0166 |
| | | polyprotein gene (AF100463) |
| 1406) | DV_t2_strain_C0166_polyprotein_gene(AF100463)_17mers_1MM.txt | DV t2 strain C0166 |
| | | polyprotein gene (AF100463) |
| 1407) | DV_t2_strain_C0166_polyprotein_gene(AF100463)_17mers_2MM.txt | DV t2 strain C0166 |
| | | polyprotein gene (AF100463) |
| 1408) | DV_t2_strain_C0166_polyprotein_gene(AF100463)_18mers_0MM.txt | DV t2 strain C0166 |
| | | polyprotein gene (AF100463) |
| 1409) | DV_t2_strain_C0166_polyprotein_gene(AF100463)_18mers_1MM.txt | DV t2 strain C0166 |
| | | polyprotein gene (AF100463) |
| 1410) | DV_t2_strain_C0166_polyprotein_gene(AF100463)_18mers_2MM.txt | DV t2 strain C0166 |
| | | polyprotein gene (AF100463) |
| 1411) | DV_t2_strain_C0167_polyprotein_gene(AF100464)_16mers_0MM.txt | DV t2 strain C0167 |
| | | polyprotein gene (AF100464) |
| 1412) | DV_t2_strain_C0167_polyprotein_gene(AF100464)_16mers_1MM.txt | DV t2 strain C0167 |
| | | polyprotein gene (AF100464) |
| 1413) | DV_t2_strain_C0167_polyprotein_gene(AF100464)_16mers_2MM.txt | DV t2 strain C0167 |
| | | polyprotein gene (AF100464) |
| 1414) | DV_t2_strain_C0167_polyprotein_gene(AF100464)_17mers_0MM.txt | DV t2 strain C0167 |
| | | polyprotein gene (AF100464) |
| 1415) | DV_t2_strain_C0167_polyprotein_gene(AF100464)_17mers_1MM.txt | DV t2 strain C0167 |
| | | polyprotein gene (AF100464) |
| 1416) | DV_t2_strain_C0167_polyprotein_gene(AF100464)_17mers_2MM.txt | DV t2 strain C0167 |
| | | polyprotein gene (AF100464) |
| 1417) | DV_t2_strain_C0167_polyprotein_gene(AF100464)_18mers_0MM.txt | DV t2 strain C0167 |
| | | polyprotein gene (AF100464) |
| 1418) | DV_t2_strain_C0167_polyprotein_gene(AF100464)_18mers_1MM.txt | DV t2 strain C0167 |
| | | polyprotein gene (AF100464) |
| 1419) | DV_t2_strain_C0167_polyprotein_gene(AF100464)_18mers_2MM.txt | DV t2 strain C0167 |
| | | polyprotein gene (AF100464) |
| 1420) | DV_t2_strain_C0371_polyprotein_gene(AF100461)_16mers_0MM.txt | DV t2 strain C0371 |
| | | polyprotein gene (AF100461) |
| 1421) | DV_t2_strain_C0371_polyprotein_gene(AF100461)_16mers_1MM.txt | DV t2 strain C0371 |
| | | polyprotein gene (AF100461) |
| 1422) | DV_t2_strain_C0371_polyprotein_gene(AF100461)_16mers_2MM.txt | DV t2 strain C0371 |
| | | polyprotein gene (AF100461) |
| 1423) | DV_t2_strain_C0371_polyprotein_gene(AF100461)_17mers_0MM.txt | DV t2 strain C0371 |
| | | polyprotein gene (AF100461) |
| 1424) | DV_t2_strain_C0371_polyprotein_gene(AF100461)_17mers_1MM.txt | DV t2 strain C0371 |
| | | polyprotein gene (AF100461) |
| 1425) | DV_t2_strain_C0371_polyprotein_gene(AF100461)_17mers_2MM.txt | DV t2 strain C0371 |
| | | polyprotein gene (AF100461) |
| 1426) | DV_t2_strain_C0371_polyprotein_gene(AF100461)_18mers_0MM.txt | DV t2 strain C0371 |
| | | polyprotein gene (AF100461) |
| 1427) | DV_t2_strain_C0371_polyprotein_gene(AF100461)_18mers_1MM.txt | DV t2 strain C0371 |
| | | polyprotein gene (AF100461) |
| 1428) | DV_t2_strain_C0371_polyprotein_gene(AF100461)_18mers_2MM.txt | DV t2 strain C0371 |
| | | polyprotein gene (AF100461) |
| 1429) | DV_t2_strain_C0390_polyprotein_gene(AF100462)_16mers_0MM.txt | DV t2 strain C0390 |
| | | polyprotein gene (AF100462) |
| 1430) | DV_t2_strain_C0390_polyprotein_gene(AF100462)_16mers_1MM.txt | DV t2 strain C0390 |
| | | polyprotein gene (AF100462) |
| 1431) | DV_t2_strain_C0390_polyprotein_gene(AF100462)_16mers_2MM.txt | DV t2 strain C0390 |
| | | polyprotein gene (AF100462) |
| 1432) | DV_t2_strain_C0390_polyprotein_gene(AF100462)_17mers_0MM.txt | DV t2 strain C0390 |
| | | polyprotein gene (AF100462) |
| 1433) | DV_t2_strain_C0390_polyprotein_gene(AF100462)_17mers_1MM.txt | DV t2 strain C0390 |
| | | polyprotein gene (AF100462) |
| 1434) | DV_t2_strain_C0390_polyprotein_gene(AF100462)_17mers_2MM.txt | DV t2 strain C0390 |
| | | polyprotein gene (AF100462) |
| 1435) | DV_t2_strain_C0390_polyprotein_gene(AF100462)_18mers_0MM.txt | DV t2 strain C0390 |
| | | polyprotein gene (AF100462) |
| 1436) | DV_t2_strain_C0390_polyprotein_gene(AF100462)_18mers_1MM.txt | DV t2 strain C0390 |
| | | polyprotein gene (AF100462) |
| 1437) | DV_t2_strain_C0390_polyprotein_gene(AF100462)_18mers_2MM.txt | DV t2 strain C0390 |
| | | polyprotein gene (AF100462) |
| 1438) | DV_t2_strain_DEN2-H-IMTSSA-MART-98-703(AF208496)_16mers_0MM.txt | DV t2 strain |
| | | DEN2-H-IMTSSA-MART-98-703 (AF208496) |
| 1439) | DV_t2_strain_DEN2-H-IMTSSA-MART-98-703(AF208496)_16mers_1MM.txt | DV t2 strain |
| | | DEN2-H-IMTSSA-MART-98-703 (AF208496) |
| 1440) | DV_t2_strain_DEN2-H-IMTSSA-MART-98-703(AF208496)_16mers_2MM.txt | DV t2 strain |
| | | DEN2-H-IMTSSA-MART-98-703 (AF208496) |
| 1441) | DV_t2_strain_DEN2-H-IMTSSA-MART-98-703(AF208496)_17mers_0MM.txt | DV t2 strain |
| | | DEN2-H-IMTSSA-MART-98-703 (AF208496) |
| 1442) | DV_t2_strain_DEN2-H-IMTSSA-MART-98-703(AF208496)_17mers_1MM.txt | DV t2 strain |
| | | DEN2-H-IMTSSA-MART-98-703 (AF208496) |
| 1443) | DV_t2_strain_DEN2-H-IMTSSA-MART-98-703(AF208496)_17mers_2MM.txt | DV t2 strain |
| | | DEN2-H-IMTSSA-MART-98-703 (AF208496) |
| 1444) | DV_t2_strain_DEN2-H-IMTSSA-MART-98-703(AF208496)_18mers_0MM.txt | DV t2 strain |
| | | DEN2-H-IMTSSA-MART-98-703 (AF208496) |
| 1445) | DV_t2_strain_DEN2-H-IMTSSA-MART-98-703(AF208496)_18mers_1MM.txt | DV t2 strain |
| | | DEN2-H-IMTSSA-MART-98-703 (AF208496) |
| 1446) | DV_t2_strain_DEN2-H-IMTSSA-MART-98-703(AF208496)_18mers_2MM.txt | DV t2 strain |
| | | DEN2-H-IMTSSA-MART-98-703 (AF208496) |
| 1447) | DV_t2_strain_FJ-10(AF276619)_16mers_0MM.txt | DV t2 strain FJ-10 (AF276619) |
| 1448) | DV_t2_strain_FJ-10(AF276619)_16mers_1MM.txt | DV t2 strain FJ-10 (AF276619) |
| 1449) | DV_t2_strain_FJ-10(AF276619)_16mers_2MM.txt | DV t2 strain FJ-10 (AF276619) |
| 1450) | DV_t2_strain_FJ-10(AF276619)_17mers_0MM.txt | DV t2 strain FJ-10 (AF276619) |
| 1451) | DV_t2_strain_FJ-10(AF276619)_17mers_1MM.txt | DV t2 strain FJ-10 (AF276619) |
| 1452) | DV_t2_strain_FJ-10(AF276619)_17mers_2MM.txt | DV t2 strain FJ-10 (AF276619) |
| 1453) | DV_t2_strain_FJ-10(AF276619)_18mers_0MM.txt | DV t2 strain FJ-10 (AF276619) |
| 1454) | DV_t2_strain_FJ-10(AF276619)_18mers_1MM.txt | DV t2 strain FJ-10 (AF276619) |
| 1455) | DV_t2_strain_FJ-10(AF276619)_18mers_2MM.txt | DV t2 strain FJ-10 (AF276619) |
| 1456) | DV_t2_strain_FJ11-99(AF359579)_16mers_0MM.txt | DV t2 strain FJ11-99 (AF359579) |
| 1457) | DV_t2_strain_FJ11-99(AF359579)_16mers_1MM.txt | DV t2 strain FJ11-99 (AF359579) |
| 1458) | DV_t2_strain_FJ11-99(AF359579)_16mers_2MM.txt | DV t2 strain FJ11-99 (AF359579) |
| 1459) | DV_t2_strain_FJ11-99(AF359579)_17mers_0MM.txt | DV t2 strain FJ11-99 (AF359579) |
| 1460) | DV_t2_strain_FJ11-99(AF359579)_17mers_1MM.txt | DV t2 strain FJ11-99 (AF359579) |
| 1461) | DV_t2_strain_FJ11-99(AF359579)_17mers_2MM.txt | DV t2 strain FJ11-99 (AF359579) |
| 1462) | DV_t2_strain_FJ11-99(AF359579)_18mers_0MM.txt | DV t2 strain FJ11-99 (AF359579) |
| 1463) | DV_t2_strain_FJ11-99(AF359579)_18mers_1MM.txt | DV t2 strain FJ11-99 (AF359579) |
| 1464) | DV_t2_strain_FJ11-99(AF359579)_18mers_2MM.txt | DV t2 strain FJ11-99 (AF359579) |
| 1465) | DV_t2_strain_IQT1797_polyprotein_gene(AF100467)_16mers_0MM.txt | DV t2 strain IQT1797 |
| | | polyprotein gene (AF100467) |
| 1466) | DV_t2_strain_IQT1797_polyprotein_gene(AF100467)_16mers_1MM.txt | DV t2 strain IQT1797 |
| | | polyprotein gene (AF100467) |
| 1467) | DV_t2_strain_IQT1797_polyprotein_gene(AF100467)_16mers_2MM.txt | DV t2 strain IQT1797 |
| | | polyprotein gene (AF100467) |
| 1468) | DV_t2_strain_IQT1797_polyprotein_gene(AF100467)_17mers_0MM.txt | DV t2 strain IQT1797 |
| | | polyprotein gene (AF100467) |
| 1469) | DV_t2_strain_IQT1797_polyprotein_gene(AF100467)_17mers_1MM.txt | DV t2 strain IQT1797 |
| | | polyprotein gene (AF100467) |
| 1470) | DV_t2_strain_IQT1797_polyprotein_gene(AF100467)_17mers_2MM.txt | DV t2 strain IQT1797 |
| | | polyprotein gene (AF100467) |
| 1471) | DV_t2_strain_IQT1797_polyprotein_gene(AF100467)_18mers_0MM.txt | DV t2 strain IQT1797 |
| | | polyprotein gene (AF100467) |
| 1472) | DV_t2_strain_IQT1797_polyprotein_gene(AF100467)_18mers_1MM.txt | DV t2 strain IQT1797 |
| | | polyprotein gene (AF100467) |
| 1473) | DV_t2_strain_IQT1797_polyprotein_gene(AF100467)_18mers_2MM.txt | DV t2 strain IQT1797 |
| | | polyprotein gene (AF100467) |
| 1474) | DV_t2_strain_IQT2913_polyprotein_gene(AF100468)_16mers_0MM.txt | DV t2 strain IQT2913 |
| | | polyprotein gene (AF100468) |
| 1475) | DV_t2_strain_IQT2913_polyprotein_gene(AF100468)_16mers_1MM.txt | DV t2 strain IQT2913 |
| | | polyprotein gene (AF100468) |
| 1476) | DV_t2_strain_IQT2913_polyprotein_gene(AF100468)_16mers_2MM.txt | DV t2 strain IQT2913 |
| | | polyprotein gene (AF100468) |
| 1477) | DV_t2_strain_IQT2913_polyprotein_gene(AF100468)_17mers_0MM.txt | DV t2 strain IQT2913 |
| | | polyprotein gene (AF100468) |
| 1478) | DV_t2_strain_IQT2913_polyprotein_gene(AF100468)_17mers_1MM.txt | DV t2 strain IQT2913 |
| | | polyprotein gene (AF100468) |
| 1479) | DV_t2_strain_IQT2913_polyprotein_gene(AF100468)_17mers_2MM.txt | DV t2 strain IQT2913 |
| | | polyprotein gene (AF100468) |
| 1480) | DV_t2_strain_IQT2913_polyprotein_gene(AF100468)_18mers_0MM.txt | DV t2 strain IQT2913 |
| | | polyprotein gene (AF100468) |
| 1481) | DV_t2_strain_IQT2913_polyprotein_gene(AF100468)_18mers_1MM.txt | DV t2 strain IQT2913 |
| | | polyprotein gene (AF100468) |
| 1482) | DV_t2_strain_IQT2913_polyprotein_gene(AF100468)_18mers_2MM.txt | DV t2 strain IQT2913 |
| | | polyprotein gene (AF100468) |
| 1483) | DV_t2_strain_K0008_polyprotein_gene(AF100459)_16mers_0MM.txt | DV t2 strain K0008 |
| | | polyprotein gene (AF100459) |
| 1484) | DV_t2_strain_K0008_polyprotein_gene(AF100459)_16mers_1MM.txt | DV t2 strain K0008 |
| | | polyprotein gene (AF100459) |
| 1485) | DV_t2_strain_K0008_polyprotein_gene(AF100459)_16mers_2MM.txt | DV t2 strain K0008 |
| | | polyprotein gene (AF100459) |
| 1486) | DV_t2_strain_K0008_polyprotein_gene(AF100459)_17mers_0MM.txt | DV t2 strain K0008 |
| | | polyprotein gene (AF100459) |
| 1487) | DV_t2_strain_K0008_polyprotein_gene(AF100459)_17mers_1MM.txt | DV t2 strain K0008 |
| | | polyprotein gene (AF100459) |
| 1488) | DV_t2_strain_K0008_polyprotein_gene(AF100459)_17mers_2MM.txt | DV t2 strain K0008 |
| | | polyprotein gene (AF100459) |
| 1489) | DV_t2_strain_K0008_polyprotein_gene(AF100459)_18mers_0MM.txt | DV t2 strain K0008 |
| | | polyprotein gene (AF100459) |
| 1490) | DV_t2_strain_K0008_polyprotein_gene(AF100459)_18mers_1MM.txt | DV t2 strain K0008 |
| | | polyprotein gene (AF100459) |
| 1491) | DV_t2_strain_K0008_polyprotein_gene(AF100459)_18mers_2MM.txt | DV t2 strain K0008 |
| | | polyprotein gene (AF100459) |
| 1492) | DV_t2_strain_K0010_polyprotein_gene(AF100460)_16mers_0MM.txt | DV t2 strain K0010 |
| | | polyprotein gene (AF100460) |
| 1493) | DV_t2_strain_K0010_polyprotein_gene(AF100460)_16mers_1MM.txt | DV t2 strain K0010 |
| | | polyprotein gene (AF100460) |
| 1494) | DV_t2_strain_K0010_polyprotein_gene(AF100460)_16mers_2MM.txt | DV t2 strain K0010 |
| | | polyprotein gene (AF100460) |
| 1495) | DV_t2_strain_K0010_polyprotein_gene(AF100460)_17mers_0MM.txt | DV t2 strain K0010 |
| | | polyprotein gene (AF100460) |
| 1496) | DV_t2_strain_K0010_polyprotein_gene(AF100460)_17mers_1MM.txt | DV t2 strain K0010 |
| | | polyprotein gene (AF100460) |
| 1497) | DV_t2_strain_K0010_polyprotein_gene(AF100460)_17mers_2MM.txt | DV t2 strain K0010 |
| | | polyprotein gene (AF100460) |
| 1498) | DV_t2_strain_K0010_polyprotein_gene(AF100460)_18mers_0MM.txt | DV t2 strain K0010 |
| | | polyprotein gene (AF100460) |
| 1499) | DV_t2_strain_K0010_polyprotein_gene(AF100460)_18mers_1MM.txt | DV t2 strain K0010 |
| | | polyprotein gene (AF100460) |
| 1500) | DV_t2_strain_K0010_polyprotein_gene(AF100460)_18mers_2MM.txt | DV t2 strain K0010 |
| | | polyprotein gene (AF100460) |
| 1501) | DV_t2_strain_Mara4_polyprotein_gene(AF100466)_16mers_0MM.txt | DV t2 strain Mara4 |
| | | polyprotein gene (AF100466) |
| 1502) | DV_t2_strain_Mara4_polyprotein_gene(AF100466)_16mers_1MM.txt | DV t2 strain Mara4 |
| | | polyprotein gene (AF100466) |
| 1503) | DV_t2_strain_Mara4_polyprotein_gene(AF100466)_16mers_2MM.txt | DV t2 strain Mara4 |
| | | polyprotein gene (AF100466) |
| 1504) | DV_t2_strain_Mara4_polyprotein_gene(AF100466)_17mers_0MM.txt | DV t2 strain Mara4 |
| | | polyprotein gene (AF100466) |
| 1505) | DV_t2_strain_Mara4_polyprotein_gene(AF100466)_17mers_1MM.txt | DV t2 strain Mara4 |
| | | polyprotein gene (AF100466) |
| 1506) | DV_t2_strain_Mara4_polyprotein_gene(AF100466)_17mers_2MM.txt | DV t2 strain Mara4 |
| | | polyprotein gene (AF100466) |
| 1507) | DV_t2_strain_Mara4_polyprotein_gene(AF100466)_18mers_0MM.txt | DV t2 strain Mara4 |
| | | polyprotein gene (AF100466) |
| 1508) | DV_t2_strain_Mara4_polyprotein_gene(AF100466)_18mers_1MM.txt | DV t2 strain Mara4 |
| | | polyprotein gene (AF100466) |
| 1509) | DV_t2_strain_Mara4_polyprotein_gene(AF100466)_18mers_2MM.txt | DV t2 strain Mara4 |
| | | polyprotein gene (AF100466) |
| 1510) | DV_t2_strain_New_Guinea_C_polyprotein_mRNA(AF038403)_16mers_0MM.txt | DV t2 strain New Guinea C |
| | | polyprotein mRNA (AF038403) |
| 1511) | DV_t2_strain_New_Guinea_C_polyprotein_mRNA(AF038403)_16mers_1MM.txt | DV t2 strain New Guinea C |
| | | polyprotein mRNA (AF038403) |
| 1512) | DV_t2_strain_New_Guinea_C_polyprotein_mRNA(AF038403)_16mers_2MM.txt | DV t2 strain New Guinea C |
| | | polyprotein mRNA (AF038403) |
| 1513) | DV_t2_strain_New_Guinea_C_polyprotein_mRNA(AF038403)_17mers_0MM.txt | DV t2 strain New Guinea C |
| | | polyprotein mRNA (AF038403) |
| 1514) | DV_t2_strain_New_Guinea_C_polyprotein_mRNA(AF038403)_17mers_1MM.txt | DV t2 strain New Guinea C |
| | | polyprotein mRNA (AF038403) |
| 1515) | DV_t2_strain_New_Guinea_C_polyprotein_mRNA(AF038403)_17mers_2MM.txt | DV t2 strain New Guinea C |
| | | polyprotein mRNA (AF038403) |
| 1516) | DV_t2_strain_New_Guinea_C_polyprotein_mRNA(AF038403)_18mers_0MM.txt | DV t2 strain New Guinea C |
| | | polyprotein mRNA (AF038403) |
| 1517) | DV_t2_strain_New_Guinea_C_polyprotein_mRNA(AF038403)_18mers_1MM.txt | DV t2 strain New Guinea C |
| | | polyprotein mRNA (AF038403) |
| 1518) | DV_t2_strain_New_Guinea_C_polyprotein_mRNA(AF038403)_18mers_2MM.txt | DV t2 strain New Guinea C |
| | | polyprotein mRNA (AF038403) |
| 1519) | DV_t2_strain_New_Guinea_C-PUO-218_hybrid_polyprotein_gene(AF038402)_16mers_0MM.txt | DV t2 strain New Guinea |
| | | C-PUO-218 hybrid polyprotein gene |
| | | (AF038402) |
| 1520) | DV_t2_strain_New_Guinea_C-PUO-218_hybrid_polyprotein_gene(AF038402)_16mers_1MM.txt | DV t2 strain New Guinea |
| | | C-PUO-218 hybrid polyprotein gene |
| | | (AF038402) |
| 1521) | DV_t2_strain_New_Guinea_C-PUO-218_hybrid_polyprotein_gene(AF038402)_16mers_2MM.txt | DV t2 strain New Guinea |
| | | C-PUO-218 hybrid polyprotein gene |
| | | (AF038402) |
| 1522) | DV_t2_strain_New_Guinea_C-PUO-218_hybrid_polyprotein_gene(AF038402)_17mers_0MM.txt | DV t2 strain New Guinea |
| | | C-PUO-218 hybrid polyprotein gene |
| | | (AF038402) |
| 1523) | DV_t2_strain_New_Guinea_C-PUO-218_hybrid_polyprotein_gene(AF038402)_17mers_1MM.txt | DV t2 strain New Guinea |
| | | C-PUO-218 hybrid polyprotein gene |
| | | (AF038402) |
| 1524) | DV_t2_strain_New_Guinea_C-PUO-218_hybrid_polyprotein_gene(AF038402)_17mers_2MM.txt | DV t2 strain New Guinea |
| | | C-PUO-218 hybrid polyprotein gene |
| | | (AF038402) |
| 1525) | DV_t2_strain_New_Guinea_C-PUO-218_hybrid_polyprotein_gene(AF038402)_18mers_0MM.txt | DV t2 strain New Guinea |
| | | C-PUO-218 hybrid polyprotein gene |
| | | (AF038402) |
| 1526) | DV_t2_strain_New_Guinea_C-PUO-218_hybrid_polyprotein_gene(AF038402)_18mers_1MM.txt | DV t2 strain New Guinea |
| | | C-PUO-218 hybrid polyprotein gene |
| | | (AF038402) |
| 1527) | DV_t2_strain_New_Guinea_C-PUO-218_hybrid_polyprotein_gene(AF038402)_18mers_2MM.txt | DV t2 strain New Guinea |
| | | C-PUO-218 hybrid polyprotein gene |
| | | (AF038402) |
| 1528) | DV_t2_strain_ThNH-11-93(AF022437)_16mers_0MM.txt | DV t2 strain ThNH-11-93 |
| | | (AF022437) |
| 1529) | DV_t2_strain_ThNH-11-93(AF022437)_16mers_1MM.txt | DV t2 strain ThNH-11-93 |
| | | (AF022437) |
| 1530) | DV_t2_strain_ThNH-11-93(AF022437)_16mers_2MM.txt | DV t2 strain ThNH-11-93 |
| | | (AF022437) |
| 1531) | DV_t2_strain_ThNH-11-93(AF022437)_17mers_0MM.txt | DV t2 strain ThNH-11-93 |
| | | (AF022437) |
| 1532) | DV_t2_strain_ThNH-11-93(AF022437)_17mers_1MM.txt | DV t2 strain ThNH-11-93 |
| | | (AF022437) |
| 1533) | DV_t2_strain_ThNH-11-93(AF022437)_17mers_2MM.txt | DV t2 strain ThNH-11-93 |
| | | (AF022437) |
| 1534) | DV_t2_strain_ThNH-11-93(AF022437)_18mers_0MM.txt | DV t2 strain ThNH-11-93 |
| | | (AF022437) |
| 1535) | DV_t2_strain_ThNH-11-93(AF022437)_18mers_1MM.txt | DV t2 strain ThNH-11-93 |
| | | (AF022437) |
| 1536) | DV_t2_strain_ThNH-11-93(AF022437)_18mers_2MM.txt | DV t2 strain ThNH-11-93 |
| | | (AF022437) |
| 1537) | DV_t2_strain_ThNH-12-93(AF022438)_16mers_0MM.txt | DV t2 strain ThNH-12-93 |
| | | (AF022438) |
| 1538) | DV_t2_strain_ThNH-12-93(AF022438)_16mers_1MM.txt | DV t2 strain ThNH-12-93 |
| | | (AF022438) |
| 1539) | DV_t2_strain_ThNH-12-93(AF022438)_16mers_2MM.txt | DV t2 strain ThNH-12-93 |
| | | (AF022438) |
| 1540) | DV_t2_strain_ThNH-12-93(AF022438)_17mers_0MM.txt | DV t2 strain ThNH-12-93 |
| | | (AF022438) |
| 1541) | DV_t2_strain_ThNH-12-93(AF022438)_17mers_1MM.txt | DV t2 strain ThNH-12-93 |
| | | (AF022438) |
| 1542) | DV_t2_strain_ThNH-12-93(AF022438)_17mers_2MM.txt | DV t2 strain ThNH-12-93 |
| | | (AF022438) |
| 1543) | DV_t2_strain_ThNH-12-93(AF022438)_18mers_0MM.txt | DV t2 strain ThNH-12-93 |
| | | (AF022438) |
| 1544) | DV_t2_strain_ThNH-12-93(AF022438)_18mers_1MM.txt | DV t2 strain ThNH-12-93 |
| | | (AF022438) |
| 1545) | DV_t2_strain_ThNH-12-93(AF022438)_18mers_2MM.txt | DV t2 strain ThNH-12-93 |
| | | (AF022438) |
| 1546) | DV_t2_strain_ThNH-14-93(AF022439)_16mers_0MM.txt | DV t2 strain ThNH-14-93 |
| | | (AF022439) |
| 1547) | DV_t2_strain_ThNH-14-93(AF022439)_16mers_1MM.txt | DV t2 strain ThNH-14-93 |
| | | (AF022439) |
| 1548) | DV_t2_strain_ThNH-14-93(AF022439)_16mers_2MM.txt | DV t2 strain ThNH-14-93 |
| | | (AF022439) |
| 1549) | DV_t2_strain_ThNH-14-93(AF022439)_17mers_0MM.txt | DV t2 strain ThNH-14-93 |
| | | (AF022439) |
| 1550) | DV_t2_strain_ThNH-14-93(AF022439)_17mers_1MM.txt | DV t2 strain ThNH-14-93 |
| | | (AF022439) |
| 1551) | DV_t2_strain_ThNH-14-93(AF022439)_17mers_2MM.txt | DV t2 strain ThNH-14-93 |
| | | (AF022439) |
| 1552) | DV_t2_strain_ThNH-14-93(AF022439)_18mers_0MM.txt | DV t2 strain ThNH-14-93 |
| | | (AF022439) |
| 1553) | DV_t2_strain_ThNH-14-93(AF022439)_18mers_1MM.txt | DV t2 strain ThNH-14-93 |
| | | (AF022439) |
| 1554) | DV_t2_strain_ThNH-14-93(AF022439)_18mers_2MM.txt | DV t2 strain ThNH-14-93 |
| | | (AF022439) |
| 1555) | DV_t2_strain_ThNH-16-93(AF022440)_16mers_0MM.txt | DV t2 strain ThNH-16-93 |
| | | (AF022440) |
| 1556) | DV_t2_strain_ThNH-16-93(AF022440)_16mers_1MM.txt | DV t2 strain ThNH-16-93 |
| | | (AF022440) |
| 1557) | DV_t2_strain_ThNH-16-93(AF022440)_16mers_2MM.txt | DV t2 strain ThNH-16-93 |
| | | (AF022440) |
| 1558) | DV_t2_strain_ThNH-16-93(AF022440)_17mers_0MM.txt | DV t2 strain ThNH-16-93 |
| | | (AF022440) |
| 1559) | DV_t2_strain_ThNH-16-93(AF022440)_17mers_1MM.txt | DV t2 strain ThNH-16-93 |
| | | (AF022440) |
| 1560) | DV_t2_strain_ThNH-16-93(AF022440)_17mers_2MM.txt | DV t2 strain ThNH-16-93 |
| | | (AF022440) |
| 1561) | DV_t2_strain_ThNH-16-93(AF022440)_18mers_0MM.txt | DV t2 strain ThNH-16-93 |
| | | (AF022440) |
| 1562) | DV_t2_strain_ThNH-16-93(AF022440)_18mers_1MM.txt | DV t2 strain ThNH-16-93 |
| | | (AF022440) |
| 1563) | DV_t2_strain_ThNH-16-93(AF022440)_18mers_2MM.txt | DV t2 strain ThNH-16-93 |
| | | (AF022440) |
| 1564) | DV_t2_strain_ThNH-28-93(AF022435)_16mers_0MM.txt | DV t2 strain ThNH-28-93 |
| | | (AF022435) |
| 1565) | DV_t2_strain_ThNH-28-93(AF022435)_16mers_1MM.txt | DV t2 strain ThNH-28-93 |
| | | (AF022435) |
| 1566) | DV_t2_strain_ThNH-28-93(AF022435)_16mers_2MM.txt | DV t2 strain ThNH-28-93 |
| | | (AF022435) |
| 1567) | DV_t2_strain_ThNH-28-93(AF022435)_17mers_0MM.txt | DV t2 strain ThNH-28-93 |
| | | (AF022435) |
| 1568) | DV_t2_strain_ThNH-28-93(AF022435)_17mers_1MM.txt | DV t2 strain ThNH-28-93 |
| | | (AF022435) |
| 1569) | DV_t2_strain_ThNH-28-93(AF022435)_17mers_2MM.txt | DV t2 strain ThNH-28-93 |
| | | (AF022435) |
| 1570) | DV_t2_strain_ThNH-28-93(AF022435)_18mers_0MM.txt | DV t2 strain ThNH-28-93 |
| | | (AF022435) |
| 1571) | DV_t2_strain_ThNH-28-93(AF022435)_18mers_1MM.txt | DV t2 strain ThNH-28-93 |
| | | (AF022435) |
| 1572) | DV_t2_strain_ThNH-28-93(AF022435)_18mers_2MM.txt | DV t2 strain ThNH-28-93 |
| | | (AF022435) |
| 1573) | DV_t2_strain_ThNH-29-93(AF169678)_16mers_0MM.txt | DV t2 strain ThNH-29-93 |
| | | (AF169678) |
| 1574) | DV_t2_strain_ThNH-29-93(AF169678)_16mers_1MM.txt | DV t2 strain ThNH-29-93 |
| | | (AF169678) |
| 1575) | DV_t2_strain_ThNH-29-93(AF169678)_16mers_2MM.txt | DV t2 strain ThNH-29-93 |
| | | (AF169678) |
| 1576) | DV_t2_strain_ThNH-29-93(AF169678)_17mers_0MM.txt | DV t2 strain ThNH-29-93 |
| | | (AF169678) |
| 1577) | DV_t2_strain_ThNH-29-93(AF169678)_17mers_1MM.txt | DV t2 strain ThNH-29-93 |
| | | (AF169678) |
| 1578) | DV_t2_strain_ThNH-29-93(AF169678)_17mers_2MM.txt | DV t2 strain ThNH-29-93 |
| | | (AF169678) |
| 1579) | DV_t2_strain_ThNH-29-93(AF169678)_18mers_0MM.txt | DV t2 strain ThNH-29-93 |
| | | (AF169678) |
| 1580) | DV_t2_strain_ThNH-29-93(AF169678)_18mers_1MM.txt | DV t2 strain ThNH-29-93 |
| | | (AF169678) |
| 1581) | DV_t2_strain_ThNH-29-93(AF169678)_18mers_2MM.txt | DV t2 strain ThNH-29-93 |
| | | (AF169678) |
| 1582) | DV_t2_strain_ThNH-36-93(AF022441)_16mers_0MM.txt | DV t2 strain ThNH-36-93 |
| | | (AF022441) |
| 1583) | DV_t2_strain_ThNH-36-93(AF022441)_16mers_1MM.txt | DV t2 strain ThNH-36-93 |
| | | (AF022441) |
| 1584) | DV_t2_strain_ThNH-36-93(AF022441)_16mers_2MM.txt | DV t2 strain ThNH-36-93 |
| | | (AF022441) |
| 1585) | DV_t2_strain_ThNH-36-93(AF022441)_17mers_0MM.txt | DV t2 strain ThNH-36-93 |
| | | (AF022441) |
| 1586) | DV_t2_strain_ThNH-36-93(AF022441)_17mers_1MM.txt | DV t2 strain ThNH-36-93 |
| | | (AF022441) |
| 1587) | DV_t2_strain_ThNH-36-93(AF022441)_17mers_2MM.txt | DV t2 strain ThNH-36-93 |
| | | (AF022441) |
| 1588) | DV_t2_strain_ThNH-36-93(AF022441)_18mers_0MM.txt | DV t2 strain ThNH-36-93 |
| | | (AF022441) |
| 1589) | DV_t2_strain_ThNH-36-93(AF022441)_18mers_1MM.txt | DV t2 strain ThNH-36-93 |
| | | (AF022441) |
| 1590) | DV_t2_strain_ThNH-36-93(AF022441)_18mers_2MM.txt | DV t2 strain ThNH-36-93 |
| | | (AF022441) |
| 1591) | DV_t2_strain_ThNH-36-93(AF169679)_16mers_0MM.txt | DV t2 strain ThNH-36-93 |
| | | (AF169679) |
| 1592) | DV_t2_strain_ThNH-36-93(AF169679)_16mers_1MM.txt | DV t2 strain ThNH-36-93 |
| | | (AF169679) |
| 1593) | DV_t2_strain_ThNH-36-93(AF169679)_16mers_2MM.txt | DV t2 strain ThNH-36-93 |
| | | (AF169679) |
| 1594) | DV_t2_strain_ThNH-36-93(AF169679)_17mers_0MM.txt | DV t2 strain ThNH-36-93 |
| | | (AF169679) |
| 1595) | DV_t2_strain_ThNH-36-93(AF169679)_17mers_1MM.txt | DV t2 strain ThNH-36-93 |
| | | (AF169679) |
| 1596) | DV_t2_strain_ThNH-36-93(AF169679)_17mers_2MM.txt | DV t2 strain ThNH-36-93 |
| | | (AF169679) |
| 1597) | DV_t2_strain_ThNH-36-93(AF169679)_18mers_0MM.txt | DV t2 strain ThNH-36-93 |
| | | (AF169679) |
| 1598) | DV_t2_strain_ThNH-36-93(AF169679)_18mers_1MM.txt | DV t2 strain ThNH-36-93 |
| | | (AF169679) |
| 1599) | DV_t2_strain_ThNH-36-93(AF169679)_18mers_2MM.txt | DV t2 strain ThNH-36-93 |
| | | (AF169679) |
| 1600) | DV_t2_strain_ThNH-45-93(AF169680)_16mers_0MM.txt | DV t2 strain ThNH-45-93 |
| | | (AF169680) |
| 1601) | DV_t2_strain_ThNH-45-93(AF169680)_16mers_1MM.txt | DV t2 strain ThNH-45-93 |
| | | (AF169680) |
| 1602) | DV_t2_strain_ThNH-45-93(AF169680)_16mers_2MM.txt | DV t2 strain ThNH-45-93 |
| | | (AF169680) |
| 1603) | DV_t2_strain_ThNH-45-93(AF169680)_17mers_0MM.txt | DV t2 strain ThNH-45-93 |
| | | (AF169680) |
| 1604) | DV_t2_strain_ThNH-45-93(AF169680)_17mers_1MM.txt | DV t2 strain ThNH-45-93 |
| | | (AF169680) |
| 1605) | DV_t2_strain_ThNH-45-93(AF169680)_17mers_2MM.txt | DV t2 strain ThNH-45-93 |
| | | (AF169680) |
| 1606) | DV_t2_strain_ThNH-45-93(AF169680)_18mers_0MM.txt | DV t2 strain ThNH-45-93 |
| | | (AF169680) |
| 1607) | DV_t2_strain_ThNH-45-93(AF169680)_18mers_1MM.txt | DV t2 strain ThNH-45-93 |
| | | (AF169680) |
| 1608) | DV_t2_strain_ThNH-45-93(AF169680)_18mers_2MM.txt | DV t2 strain ThNH-45-93 |
| | | (AF169680) |
| 1609) | DV_t2_strain_ThNH-52-93(AF022436)_16mers_0MM.txt | DV t2 strain ThNH-52-93 |
| | | (AF022436) |
| 1610) | DV_t2_strain_ThNH-52-93(AF022436)_16mers_1MM.txt | DV t2 strain ThNH-52-93 |
| | | (AF022436) |
| 1611) | DV_t2_strain_ThNH-52-93(AF022436)_16mers_2MM.txt | DV t2 strain ThNH-52-93 |
| | | (AF022436) |
| 1612) | DV_t2_strain_ThNH-52-93(AF022436)_17mers_0MM.txt | DV t2 strain ThNH-52-93 |
| | | (AF022436) |
| 1613) | DV_t2_strain_ThNH-52-93(AF022436)_17mers_1MM.txt | DV t2 strain ThNH-52-93 |
| | | (AF022436) |
| 1614) | DV_t2_strain_ThNH-52-93(AF022436)_17mers_2MM.txt | DV t2 strain ThNH-52-93 |
| | | (AF022436) |
| 1615) | DV_t2_strain_ThNH-52-93(AF022436)_18mers_0MM.txt | DV t2 strain ThNH-52-93 |
| | | (AF022436) |
| 1616) | DV_t2_strain_ThNH-52-93(AF022436)_18mers_1MM.txt | DV t2 strain ThNH-52-93 |
| | | (AF022436) |
| 1617) | DV_t2_strain_ThNH-52-93(AF022436)_18mers_2MM.txt | DV t2 strain ThNH-52-93 |
| | | (AF022436) |
| 1618) | DV_t2_strain_ThNH-54-93(AF169682)_16mers_0MM.txt | DV t2 strain ThNH-54-93 |
| | | (AF169682) |
| 1619) | DV_t2_strain_ThNH-54-93(AF169682)_16mers_1MM.txt | DV t2 strain ThNH-54-93 |
| | | (AF169682) |
| 1620) | DV_t2_strain_ThNH-54-93(AF169682)_16mers_2MM.txt | DV t2 strain ThNH-54-93 |
| | | (AF169682) |
| 1621) | DV_t2_strain_ThNH-54-93(AF169682)_17mers_0MM.txt | DV t2 strain ThNH-54-93 |
| | | (AF169682) |
| 1622) | DV_t2_strain_ThNH-54-93(AF169682)_17mers_1MM.txt | DV t2 strain ThNH-54-93 |
| | | (AF169682) |
| 1623) | DV_t2_strain_ThNH-54-93(AF169682)_17mers_2MM.txt | DV t2 strain ThNH-54-93 |
| | | (AF169682) |
| 1624) | DV_t2_strain_ThNH-54-93(AF169682)_18mers_0MM.txt | DV t2 strain ThNH-54-93 |
| | | (AF169682) |
| 1625) | DV_t2_strain_ThNH-54-93(AF169682)_18mers_1MM.txt | DV t2 strain ThNH-54-93 |
| | | (AF169682) |
| 1626) | DV_t2_strain_ThNH-54-93(AF169682)_18mers_2MM.txt | DV t2 strain ThNH-54-93 |
| | | (AF169682) |
| 1627) | DV_t2_strain_ThNH-55-93(AF169681)_16mers_0MM.txt | DV t2 strain ThNH-55-93 |
| | | (AF169681) |
| 1628) | DV_t2_strain_ThNH-55-93(AF169681)_16mers_1MM.txt | DV t2 strain ThNH-55-93 |
| | | (AF169681) |
| 1629) | DV_t2_strain_ThNH-55-93(AF169681)_16mers_2MM.txt | DV t2 strain ThNH-55-93 |
| | | (AF169681) |
| 1630) | DV_t2_strain_ThNH-55-93(AF169681)_17mers_0MM.txt | DV t2 strain ThNH-55-93 |
| | | (AF169681) |
| 1631) | DV_t2_strain_ThNH-55-93(AF169681)_17mers_1MM.txt | DV t2 strain ThNH-55-93 |
| | | (AF169681) |
| 1632) | DV_t2_strain_ThNH-55-93(AF169681)_17mers_2MM.txt | DV t2 strain ThNH-55-93 |
| | | (AF169681) |
| 1633) | DV_t2_strain_ThNH-55-93(AF169681)_18mers_0MM.txt | DV t2 strain ThNH-55-93 |
| | | (AF169681) |
| 1634) | DV_t2_strain_ThNH-55-93(AF169681)_18mers_1MM.txt | DV t2 strain ThNH-55-93 |
| | | (AF169681) |
| 1635) | DV_t2_strain_ThNH-55-93(AF169681)_18mers_2MM.txt | DV t2 strain ThNH-55-93 |
| | | (AF169681) |
| 1636) | DV_t2_strain_ThNH-62-93(AF169683)_16mers_0MM.txt | DV t2 strain ThNH-62-93 |
| | | (AF169683) |
| 1637) | DV_t2_strain_ThNH-62-93(AF169683)_16mers_1MM.txt | DV t2 strain ThNH-62-93 |
| | | (AF169683) |
| 1638) | DV_t2_strain_ThNH-62-93(AF169683)_16mers_2MM.txt | DV t2 strain ThNH-62-93 |
| | | (AF169683) |
| 1639) | DV_t2_strain_ThNH-62-93(AF169683)_17mers_0MM.txt | DV t2 strain ThNH-62-93 |
| | | (AF169683) |
| 1640) | DV_t2_strain_ThNH-62-93(AF169683)_17mers_1MM.txt | DV t2 strain ThNH-62-93 |
| | | (AF169683) |
| 1641) | DV_t2_strain_ThNH-62-93(AF169683)_17mers_2MM.txt | DV t2 strain ThNH-62-93 |
| | | (AF169683) |
| 1642) | DV_t2_strain_ThNH-62-93(AF169683)_18mers_0MM.txt | DV t2 strain ThNH-62-93 |
| | | (AF169683) |
| 1643) | DV_t2_strain_ThNH-62-93(AF169683)_18mers_1MM.txt | DV t2 strain ThNH-62-93 |
| | | (AF169683) |
| 1644) | DV_t2_strain_ThNH-62-93(AF169683)_18mers_2MM.txt | DV t2 strain ThNH-62-93 |
| | | (AF169683) |
| 1645) | DV_t2_strain_ThNH-63-93(AF169684)_16mers_0MM.txt | DV t2 strain ThNH-63-93 |
| | | (AF169684) |
| 1646) | DV_t2_strain_ThNH-63-93(AF169684)_16mers_1MM.txt | DV t2 strain ThNH-63-93 |
| | | (AF169684) |
| 1647) | DV_t2_strain_ThNH-63-93(AF169684)_16mers_2MM.txt | DV t2 strain ThNH-63-93 |
| | | (AF169684) |
| 1648) | DV_t2_strain_ThNH-63-93(AF169684)_17mers_0MM.txt | DV t2 strain ThNH-63-93 |
| | | (AF169684) |
| 1649) | DV_t2_strain_ThNH-63-93(AF169684)_17mers_1MM.txt | DV t2 strain ThNH-63-93 |
| | | (AF169684) |
| 1650) | DV_t2_strain_ThNH-63-93(AF169684)_17mers_2MM.txt | DV t2 strain ThNH-63-93 |
| | | (AF169684) |
| 1651) | DV_t2_strain_ThNH-63-93(AF169684)_18mers_0MM.txt | DV t2 strain ThNH-63-93 |
| | | (AF169684) |
| 1652) | DV_t2_strain_ThNH-63-93(AF169684)_18mers_1MM.txt | DV t2 strain ThNH-63-93 |
| | | (AF169684) |
| 1653) | DV_t2_strain_ThNH-63-93(AF169684)_18mers_2MM.txt | DV t2 strain ThNH-63-93 |
| | | (AF169684) |
| 1654) | DV_t2_strain_ThNH-69-93(AF169685)_16mers_0MM.txt | DV t2 strain ThNH-69-93 |
| | | (AF169685) |
| 1655) | DV_t2_strain_ThNH-69-93(AF169685)_16mers_1MM.txt | DV t2 strain ThNH-69-93 |
| | | (AF169685) |
| 1656) | DV_t2_strain_ThNH-69-93(AF169685)_16mers_2MM.txt | DV t2 strain ThNH-69-93 |
| | | (AF169685) |
| 1657) | DV_t2_strain_ThNH-69-93(AF169685)_17mers_0MM.txt | DV t2 strain ThNH-69-93 |
| | | (AF169685) |
| 1658) | DV_t2_strain_ThNH-69-93(AF169685)_17mers_1MM.txt | DV t2 strain ThNH-69-93 |
| | | (AF169685) |
| 1659) | DV_t2_strain_ThNH-69-93(AF169685)_17mers_2MM.txt | DV t2 strain ThNH-69-93 |
| | | (AF169685) |
| 1660) | DV_t2_strain_ThNH-69-93(AF169685)_18mers_0MM.txt | DV t2 strain ThNH-69-93 |
| | | (AF169685) |
| 1661) | DV_t2_strain_ThNH-69-93(AF169685)_18mers_1MM.txt | DV t2 strain ThNH-69-93 |
| | | (AF169685) |
| 1662) | DV_t2_strain_ThNH-69-93(AF169685)_18mers_2MM.txt | DV t2 strain ThNH-69-93 |
| | | (AF169685) |
| 1663) | DV_t2_strain_ThNH-73-93(AF169686)_16mers_0MM.txt | DV t2 strain ThNH-73-93 |
| | | (AF169686) |
| 1664) | DV_t2_strain_ThNH-73-93(AF169686)_16mers_1MM.txt | DV t2 strain ThNH-73-93 |
| | | (AF169686) |
| 1665) | DV_t2_strain_ThNH-73-93(AF169686)_16mers_2MM.txt | DV t2 strain ThNH-73-93 |
| | | (AF169686) |
| 1666) | DV_t2_strain_ThNH-73-93(AF169686)_17mers_0MM.txt | DV t2 strain ThNH-73-93 |
| | | (AF169686) |
| 1667) | DV_t2_strain_ThNH-73-93(AF169686)_17mers_1MM.txt | DV t2 strain ThNH-73-93 |
| | | (AF169686) |
| 1668) | DV_t2_strain_ThNH-73-93(AF169686)_17mers_2MM.txt | DV t2 strain ThNH-73-93 |
| | | (AF169686) |
| 1669) | DV_t2_strain_ThNH-73-93(AF169686)_18mers_0MM.txt | DV t2 strain ThNH-73-93 |
| | | (AF169686) |
| 1670) | DV_t2_strain_ThNH-73-93(AF169686)_18mers_1MM.txt | DV t2 strain ThNH-73-93 |
| | | (AF169686) |
| 1671) | DV_t2_strain_ThNH-73-93(AF169686)_18mers_2MM.txt | DV t2 strain ThNH-73-93 |
| | | (AF169686) |
| 1672) | DV_t2_strain_ThNH-76-93(AF169687)_16mers_0MM.txt | DV t2 strain ThNH-76-93 |
| | | (AF169687) |
| 1673) | DV_t2_strain_ThNH-76-93(AF169687)_16mers_1MM.txt | DV t2 strain ThNH-76-93 |
| | | (AF169687) |
| 1674) | DV_t2_strain_ThNH-76-93(AF169687)_16mers_2MM.txt | DV t2 strain ThNH-76-93 |
| | | (AF169687) |
| 1675) | DV_t2_strain_ThNH-76-93(AF169687)_17mers_0MM.txt | DV t2 strain ThNH-76-93 |
| | | (AF169687) |
| 1676) | DV_t2_strain_ThNH-76-93(AF169687)_17mers_1MM.txt | DV t2 strain ThNH-76-93 |
| | | (AF169687) |
| 1677) | DV_t2_strain_ThNH-76-93(AF169687)_17mers_2MM.txt | DV t2 strain ThNH-76-93 |
| | | (AF169687) |
| 1678) | DV_t2_strain_ThNH-76-93(AF169687)_18mers_0MM.txt | DV t2 strain ThNH-76-93 |
| | | (AF169687) |
| 1679) | DV_t2_strain_ThNH-76-93(AF169687)_18mers_1MM.txt | DV t2 strain ThNH-76-93 |
| | | (AF169687) |
| 1680) | DV_t2_strain_ThNH-76-93(AF169687)_18mers_2MM.txt | DV t2 strain ThNH-76-93 |
| | | (AF169687) |
| 1681) | DV_t2_strain_ThNH-7-93(AF022434)_16mers_0MM.txt | DV t2 strain ThNH-7-93 |
| | | (AF022434) |
| 1682) | DV_t2_strain_ThNH-7-93(AF022434)_16mers_1MM.txt | DV t2 strain ThNH-7-93 |
| | | (AF022434) |
| 1683) | DV_t2_strain_ThNH-7-93(AF022434)_16mers_2MM.txt | DV t2 strain ThNH-7-93 |
| | | (AF022434) |
| 1684) | DV_t2_strain_ThNH-7-93(AF022434)_17mers_0MM.txt | DV t2 strain ThNH-7-93 |
| | | (AF022434) |
| 1685) | DV_t2_strain_ThNH-7-93(AF022434)_17mers_1MM.txt | DV t2 strain ThNH-7-93 |
| | | (AF022434) |
| 1686) | DV_t2_strain_ThNH-7-93(AF022434)_17mers_2MM.txt | DV t2 strain ThNH-7-93 |
| | | (AF022434) |
| 1687) | DV_t2_strain_ThNH-7-93(AF022434)_18mers_0MM.txt | DV t2 strain ThNH-7-93 |
| | | (AF022434) |
| 1688) | DV_t2_strain_ThNH-7-93(AF022434)_18mers_1MM.txt | DV t2 strain ThNH-7-93 |
| | | (AF022434) |
| 1689) | DV_t2_strain_ThNH-7-93(AF022434)_18mers_2MM.txt | DV t2 strain ThNH-7-93 |
| | | (AF022434) |
| 1690) | DV_t2_strain_ThNH-81-93(AF169688)_16mers_0MM.txt | DV t2 strain ThNH-81-93 |
| | | (AF169688) |
| 1691) | DV_t2_strain_ThNH-81-93(AF169688)_16mers_1MM.txt | DV t2 strain ThNH-81-93 |
| | | (AF169688) |
| 1692) | DV_t2_strain_ThNH-81-93(AF169688)_16mers_2MM.txt | DV t2 strain ThNH-81-93 |
| | | (AF169688) |
| 1693) | DV_t2_strain_ThNH-81-93(AF169688)_17mers_0MM.txt | DV t2 strain ThNH-81-93 |
| | | (AF169688) |
| 1694) | DV_t2_strain_ThNH-81-93(AF169688)_17mers_1MM.txt | DV t2 strain ThNH-81-93 |
| | | (AF169688) |
| 1695) | DV_t2_strain_ThNH-81-93(AF169688)_17mers_2MM.txt | DV t2 strain ThNH-81-93 |
| | | (AF169688) |
| 1696) | DV_t2_strain_ThNH-81-93(AF169688)_18mers_0MM.txt | DV t2 strain ThNH-81-93 |
| | | (AF169688) |
| 1697) | DV_t2_strain_ThNH-81-93(AF169688)_18mers_1MM.txt | DV t2 strain ThNH-81-93 |
| | | (AF169688) |
| 1698) | DV_t2_strain_ThNH-81-93(AF169688)_18mers_2MM.txt | DV t2 strain ThNH-81-93 |
| | | (AF169688) |
| 1699) | DV_t2_strain_TSV01(AY037116)_16mers_0MM.txt | DV t2 strain TSV01 (AY037116) |
| 1700) | DV_t2_strain_TSV01(AY037116)_16mers_1MM.txt | DV t2 strain TSV01 (AY037116) |
| 1701) | DV_t2_strain_TSV01(AY037116)_16mers_2MM.txt | DV t2 strain TSV01 (AY037116) |
| 1702) | DV_t2_strain_TSV01(AY037116)_17mers_0MM.txt | DV t2 strain TSV01 (AY037116) |
| 1703) | DV_t2_strain_TSV01(AY037116)_17mers_1MM.txt | DV t2 strain TSV01 (AY037116) |
| 1704) | DV_t2_strain_TSV01(AY037116)_17mers_2MM.txt | DV t2 strain TSV01 (AY037116) |
| 1705) | DV_t2_strain_TSV01(AY037116)_18mers_0MM.txt | DV t2 strain TSV01 (AY037116) |
| 1706) | DV_t2_strain_TSV01(AY037116)_18mers_1MM.txt | DV t2 strain TSV01 (AY037116) |
| 1707) | DV_t2_strain_TSV01(AY037116)_18mers_2MM.txt | DV t2 strain TSV01 (AY037116) |
| 1708) | DV_t2_strain_Ven2_polyprotein_gene(AF100465)_16mers_0MM.txt | DV t2 strain Ven2 |
| | | polyprotein gene (AF100465) |
| 1709) | DV_t2_strain_Ven2_polyprotein_gene(AF100465)_16mers_1MM.txt | DV t2 strain Ven2 |
| | | polyprotein gene (AF100465) |
| 1710) | DV_t2_strain_Ven2_polyprotein_gene(AF100465)_16mers_2MM.txt | DV t2 strain Ven2 |
| | | polyprotein gene (AF100465) |
| 1711) | DV_t2_strain_Ven2_polyprotein_gene(AF100465)_17mers_0MM.txt | DV t2 strain Ven2 |
| | | polyprotein gene (AF100465) |
| 1712) | DV_t2_strain_Ven2_polyprotein_gene(AF100465)_17mers_1MM.txt | DV t2 strain Ven2 |
| | | polyprotein gene (AF100465) |
| 1713) | DV_t2_strain_Ven2_polyprotein_gene(AF100465)_17mers_2MM.txt | DV t2 strain Ven2 |
| | | polyprotein gene (AF100465) |
| 1714) | DV_t2_strain_Ven2_polyprotein_gene(AF100465)_18mers_0MM.txt | DV t2 strain Ven2 |
| | | polyprotein gene (AF100465) |
| 1715) | DV_t2_strain_Ven2_polyprotein_gene(AF100465)_18mers_1MM.txt | DV t2 strain Ven2 |
| | | polyprotein gene (AF100465) |
| 1716) | DV_t2_strain_Ven2_polyprotein_gene(AF100465)_18mers_2MM.txt | DV t2 strain Ven2 |
| | | polyprotein gene (AF100465) |
| 1717) | DV_t3(M939130)_16mers_0MM.txt | DV t3 (M939130) |
| 1718) | DV_t3(M939130)_16mers_1MM.txt | DV t3 (M939130) |
| 1719) | DV_t3(M939130)_16mers_2MM.txt | DV t3 (M939130) |
| 1720) | DV_t3(M939130)_17mers_0MM.txt | DV t3 (M939130) |
| 1721) | DV_t3(M939130)_17mers_1MM.txt | DV t3 (M939130) |
| 1722) | DV_t3(M939130)_17mers_2MM.txt | DV t3 (M939130) |
| 1723) | DV_t3(M939130)_18mers_0MM.txt | DV t3 (M939130) |
| 1724) | DV_t3(M939130)_18mers_1MM.txt | DV t3 (M939130) |
| 1725) | DV_t3(M939130)_18mers_2MM.txt | DV t3 (M939130) |
| 1726) | DV_t3_isolate_D3-H-IMTSSA-MART-1999-1243(AY099337)_16mers_0MM.txt | DV t3 isolate D3- |
| | | H-IMTSSA-MART-1999-1243 |
| | | (AY099337) |
| 1727) | DV_t3_isolate_D3-H-IMTSSA-MART-1999-1243(AY099337)_16mers_1MM.txt | DV t3 isolate D3- |
| | | H-IMTSSA-MART-1999-1243 |
| | | (AY099337) |
| 1728) | DV_t3_isolate_D3-H-IMTSSA-MART-1999-1243(AY099337)_16mers_2MM.txt | DV t3 isolate D3- |
| | | H-IMTSSA-MART-1999-1243 |
| | | (AY099337) |
| 1729) | DV_t3_isolate_D3-H-IMTSSA-MART-1999-1243(AY099337)_17mers_0MM.txt | DV t3 isolate D3- |
| | | H-IMTSSA-MART-1999-1243 |
| | | (AY099337) |
| 1730) | DV_t3_isolate_D3-H-IMTSSA-MART-1999-1243(AY099337)_17mers_1MM.txt | DV t3 isolate D3- |
| | | H-IMTSSA-MART-1999-1243 |
| | | (AY099337) |
| 1731) | DV_t3_isolate_D3-H-IMTSSA-MART-1999-1243(AY099337)_17mers_2MM.txt | DV t3 isolate D3- |
| | | H-IMTSSA-MART-1999-1243 |
| | | (AY099337) |
| 1732) | DV_t3_isolate_D3-H-IMTSSA-MART-1999-1243(AY099337)_18mers_0MM.txt | DV t3 isolate D3- |
| | | H-IMTSSA-MART-1999-1243 |
| | | (AY099337) |
| 1733) | DV_t3_isolate_D3-H-IMTSSA-MART-1999-1243(AY099337)_18mers_1MM.txt | DV t3 isolate D3- |
| | | H-IMTSSA-MART-1999-1243 |
| | | (AY099337) |
| 1734) | DV_t3_isolate_D3-H-IMTSSA-MART-1999-1243(AY099337)_18mers_2MM.txt | DV t3 isolate D3- |
| | | H-IMTSSA-MART-1999-1243 |
| | | (AY099337) |
| 1735) | DV_t3_isolate_D3-H-IMTSSA-SRI-2000-1266(AY099336)_16mers_0MM.txt | DV t3 isolate D3- |
| | | H-IMTSSA-SRI-2000-1266 |
| | | (AY099336) |
| 1736) | DV_t3_isolate_D3-H-IMTSSA-SRI-2000-1266(AY099336)_16mers_1MM.txt | DV t3 isolate D3- |
| | | H-IMTSSA-SRI-2000-1266 |
| | | (AY099336) |
| 1737) | DV_t3_isolate_D3-H-IMTSSA-SRI-2000-1266(AY099336)_16mers_2MM.txt | DV t3 isolate D3- |
| | | H-IMTSSA-SRI-2000-1266 |
| | | (AY099336) |
| 1738) | DV_t3_isolate_D3-H-IMTSSA-SRI-2000-1266(AY099336)_17mers_0MM.txt | DV t3 isolate D3- |
| | | H-IMTSSA-SRI-2000-1266 |
| | | (AY099336) |
| 1739) | DV_t3_isolate_D3-H-IMTSSA-SRI-2000-1266(AY099336)_17mers_1MM.txt | DV t3 isolate D3- |
| | | H-IMTSSA-SRI-2000-1266 |
| | | (AY099336) |
| 1740) | DV_t3_isolate_D3-H-IMTSSA-SRI-2000-1266(AY099336)_17mers_2MM.txt | DV t3 isolate D3- |
| | | H-IMTSSA-SRI-2000-1266 |
| | | (AY099336) |
| 1741) | DV_t3_isolate_D3-H-IMTSSA-SRI-2000-1266(AY099336)_18mers_0MM.txt | DV t3 isolate D3- |
| | | H-IMTSSA-SRI-2000-1266 |
| | | (AY099336) |
| 1742) | DV_t3_isolate_D3-H-IMTSSA-SRI-2000-1266(AY099336)_18mers_1MM.txt | DV t3 isolate D3- |
| | | H-IMTSSA-SRI-2000-1266 |
| | | (AY099336) |
| 1743) | DV_t3_isolate_D3-H-IMTSSA-SRI-2000-1266(AY099336)_18mers_2MM.txt | DV t3 isolate D3- |
| | | H-IMTSSA-SRI-2000-1266 |
| | | (AY099336) |
| 1744) | DV_t3_strain_80-2(AF317645)_16mers_0MM.txt | DV t3 strain 80-2 (AF317645) |
| 1745) | DV_t3_strain_80-2(AF317645)_16mers_1MM.txt | DV t3 strain 80-2 (AF317645) |
| 1746) | DV_t3_strain_80-2(AF317645)_16mers_2MM.txt | DV t3 strain 80-2 (AF317645) |
| 1747) | DV_t3_strain_80-2(AF317645)_17mers_0MM.txt | DV t3 strain 80-2 (AF317645) |
| 1748) | DV_t3_strain_80-2(AF317645)_17mers_1MM.txt | DV t3 strain 80-2 (AF317645) |
| 1749) | DV_t3_strain_80-2(AF317645)_17mers_2MM.txt | DV t3 strain 80-2 (AF317645) |
| 1750) | DV_t3_strain_80-2(AF317645)_18mers_0MM.txt | DV t3 strain 80-2 (AF317645) |
| 1751) | DV_t3_strain_80-2(AF317645)_18mers_1MM.txt | DV t3 strain 80-2 (AF317645) |
| 1752) | DV_t3_strain_80-2(AF317645)_18mers_2MM.txt | DV t3 strain 80-2 (AF317645) |
| 1753) | DV_t4_polyprotein_precursor(M14931)_16mers_0MM.txt | DV t4 polyprotein precursor |
| | | (M14931) |
| 1754) | DV_t4_polyprotein_precursor(M14931)_16mers_1MM.txt | DV t4 polyprotein precursor |
| | | (M14931) |
| 1755) | DV_t4_polyprotein_precursor(M14931)_16mers_2MM.txt | DV t4 polyprotein precursor |
| | | (M14931) |
| 1756) | DV_t4_polyprotein_precursor(M14931)_17mers_0MM.txt | DV t4 polyprotein precursor |
| | | (M14931) |
| 1757) | DV_t4_polyprotein_precursor(M14931)_17mers_1MM.txt | DV t4 polyprotein precursor |
| | | (M14931) |
| 1758) | DV_t4_polyprotein_precursor(M14931)_17mers_2MM.txt | DV t4 polyprotein precursor |
| | | (M14931) |
| 1759) | DV_t4_polyprotein_precursor(M14931)_18mers_0MM.txt | DV t4 polyprotein precursor |
| | | (M14931) |
| 1760) | DV_t4_polyprotein_precursor(M14931)_18mers_1MM.txt | DV t4 polyprotein precursor |
| | | (M14931) |
| 1761) | DV_t4_polyprotein_precursor(M14931)_18mers_2MM.txt | DV t4 polyprotein precursor |
| | | (M14931) |
| 1762) | DV_t4_China_Guangzhou_B5_strain_genomic_RNA(AF289029)_16mers_0MM.txt | DV t4 |
| | | China Guangzhou B5 strain |
| | | genomic RNA (AF289029) |
| 1763) | DV_t4_China_Guangzhou_B5_strain_genomic_RNA(AF289029)_16mers_1MM.txt | DV t4 |
| | | China Guangzhou B5 strain |
| | | genomic RNA (AF289029) |
| 1764) | DV_t4_China_Guangzhou_B5_strain_genomic_RNA(AF289029)_16mers_2MM.txt | DV t4 |
| | | China Guangzhou B5 strain |
| | | genomic RNA (AF289029) |
| 1765) | DV_t4_China_Guangzhou_B5_strain_genomic_RNA(AF289029)_17mers_0MM.txt | DV t4 |
| | | China Guangzhou B5 strain |
| | | genomic RNA (AF289029) |
| 1766) | DV_t4_China_Guangzhou_B5_strain_genomic_RNA(AF289029)_17mers_1MM.txt | DV t4 |
| | | China Guangzhou B5 strain |
| | | genomic RNA (AF289029) |
| 1767) | DV_t4_China_Guangzhou_B5_strain_genomic_RNA(AF289029)_17mers_2MM.txt | DV t4 |
| | | China Guangzhou B5 strain |
| | | genomic RNA (AF289029) |
| 1768) | DV_t4_China_Guangzhou_B5_strain_genomic_RNA(AF289029)_18mers_0MM.txt | DV t4 |
| | | China Guangzhou B5 strain |
| | | genomic RNA (AF289029) |
| 1769) | DV_t4_China_Guangzhou_B5_strain_genomic_RNA(AF289029)_18mers_1MM.txt | DV t4 |
| | | China Guangzhou B5 strain |
| | | genomic RNA (AF289029) |
| 1770) | DV_t4_China_Guangzhou_B5_strain_genomic_RNA(AF289029)_18mers_2MM.txt | DV t4 |
| | | China Guangzhou B5 strain |
| | | genomic RNA (AF289029) |
| 1771) | DV_t4_recombinant_clone_2A(AF375822)_16mers_0MM.txt | DV t4 recombinant clone 2A |
| | | (AF375822) |
| 1772) | DV_t4_recombinant_clone_2A(AF375822)_16mers_1MM.txt | DV t4 recombinant clone 2A |
| | | (AF375822) |
| 1773) | DV_t4_recombinant_clone_2A(AF375822)_16mers_2MM.txt | DV t4 recombinant clone 2A |
| | | (AF375822) |
| 1774) | DV_t4_recombinant_clone_2A(AF375822)_17mers_0MM.txt | DV t4 recombinant clone 2A |
| | | (AF375822) |
| 1775) | DV_t4_recombinant_clone_2A(AF375822)_17mers_1MM.txt | DV t4 recombinant clone 2A |
| | | (AF375822) |
| 1776) | DV_t4_recombinant_clone_2A(AF375822)_17mers_2MM.txt | DV t4 recombinant clone 2A |
| | | (AF375822) |
| 1777) | DV_t4_recombinant_clone_2A(AF375822)_18mers_0MM.txt | DV t4 recombinant clone 2A |
| | | (AF375822) |
| 1778) | DV_t4_recombinant_clone_2A(AF375822)_18mers_1MM.txt | DV t4 recombinant clone 2A |
| | | (AF375822) |
| 1779) | DV_t4_recombinant_clone_2A(AF375822)_18mers_2MM.txt | DV t4 recombinant clone 2A |
| | | (AF375822) |
| 1780) | DV_t4_recombinant_clone_2Ade130(AF326826)_16mers_0MM.txt | DV t4 recombinant clone |
| | | 2Ade130 (AF326826) |
| 1781) | DV_t4_recombinant_clone_2Ade130(AF326826)_16mers_1MM.txt | DV t4 recombinant clone |
| | | 2Ade130 (AF326826) |
| 1782) | DV_t4_recombinant_clone_2Ade130(AF326826)_16mers_2MM.txt | DV t4 recombinant clone |
| | | 2Ade130 (AF326826) |
| 1783) | DV_t4_recombinant_clone_2Ade130(AF326826)_17mers_0MM.txt | DV t4 recombinant clone |
| | | 2Ade130 (AF326826) |
| 1784) | DV_t4_recombinant_clone_2Ade130(AF326826)_17mers_1MM.txt | DV t4 recombinant clone |
| | | 2Ade130 (AF326826) |
| 1785) | DV_t4_recombinant_clone_2Ade130(AF326826)_17mers_2MM.txt | DV t4 recombinant clone |
| | | 2Ade130 (AF326826) |
| 1786) | DV_t4_recombinant_clone_2Ade130(AF326826)_18mers_0MM.txt | DV t4 recombinant clone |
| | | 2Ade130 (AF326826) |
| 1787) | DV_t4_recombinant_clone_2Ade130(AF326826)_18mers_1MM.txt | DV t4 recombinant clone |
| | | 2Ade130 (AF326826) |
| 1788) | DV_t4_recombinant_clone_2Ade130(AF326826)_18mers_2MM.txt | DV t4 recombinant clone |
| | | 2Ade130 (AF326826) |
| 1789) | DV_t4_recombinant_clone_rDEN4(AF326825)_16mers_0MM.txt | DV t4 recombinant clone |
| | | rDEN4 (AF326825) |
| 1790) | DV_t4_recombinant_clone_rDEN4(AF326825)_16mers_1MM.txt | DV t4 recombinant clone |
| | | rDEN4 (AF326825) |
| 1791) | DV_t4_recombinant_clone_rDEN4(AF326825)_16mers_2MM.txt | DV t4 recombinant clone |
| | | rDEN4 (AF326825) |
| 1792) | DV_t4_recombinant_clone_rDEN4(AF326825)_17mers_0MM.txt | DV t4 recombinant clone |
| | | rDEN4 (AF326825) |
| 1793) | DV_t4_recombinant_clone_rDEN4(AF326825)_17mers_1MM.txt | DV t4 recombinant clone |
| | | rDEN4 (AF326825) |
| 1794) | DV_t4_recombinant_clone_rDEN4(AF326825)_17mers_2MM.txt | DV t4 recombinant clone |
| | | rDEN4 (AF326825) |
| 1795) | DV_t4_recombinant_clone_rDEN4(AF326825)_18mers_0MM.txt | DV t4 recombinant clone |
| | | rDEN4 (AF326825) |
| 1796) | DV_t4_recombinant_clone_rDEN4(AF326825)_18mers_1MM.txt | DV t4 recombinant clone |
| | | rDEN4 (AF326825) |
| 1797) | DV_t4_recombinant_clone_rDEN4(AF326825)_18mers_2MM.txt | DV t4 recombinant clone |
| | | rDEN4 (AF326825) |
| 1798) | DV_t4_recombinant_clone_rDEN4de130(AF326827)_16mers_0MM.txt | DV t4 recombinant clone |
| | | rDEN4de130 (AF326827) |
| 1799) | DV_t4_recombinant_clone_rDEN4de130(AF326827)_16mers_1MM.txt | DV t4 recombinant clone |
| | | rDEN4de130 (AF326827) |
| 1800) | DV_t4_recombinant_clone_rDEN4de130(AF326827)_16mers_2MM.txt | DV t4 recombinant clone |
| | | rDEN4de130 (AF326827) |
| 1801) | DV_t4_recombinant_clone_rDEN4de130(AF326827)_17mers_0MM.txt | DV t4 recombinant clone |
| | | rDEN4de130 (AF326827) |
| 1802) | DV_t4_recombinant_clone_rDEN4de130(AF326827)_17mers_1MM.txt | DV t4 recombinant clone |
| | | rDEN4de130 (AF326827) |
| 1803) | DV_t4_recombinant_clone_rDEN4de130(AF326827)_17mers_2MM.txt | DV t4 recombinant clone |
| | | rDEN4de130 (AF326827) |
| 1804) | DV_t4_recombinant_clone_rDEN4del30(AF326827)_18mers_0MM.txt | DV t4 recombinant clone |
| | | rDEN4del30 (AF326827) |
| 1805) | DV_t4_recombinant_clone_rDEN4del30(AF326827)_18mers_1MM.txt | DV t4 recombinant clone |
| | | rDEN4del30 (AF326827) |
| 1806) | DV_t4_recombinant_clone_rDEN4del30(AF326827)_18mers_2MM.txt | DV t4 recombinant clone |
| | | rDEN4del30 (AF326827) |
| 1807) | DV_t4_strain_814669(AF326573)_16mers_0MM.txt | DV t4 strain 814669 (AF326573) |
| 1808) | DV_t4_strain_814669(AF326573)_16mers_1MM.txt | DV t4 strain 814669 (AF326573) |
| 1809) | DV_t4_strain_814669(AF326573)_16mers_2MM.txt | DV t4 strain 814669 (AF326573) |
| 1810) | DV_t4_strain_814669(AF326573)_17mers_0MM.txt | DV t4 strain 814669 (AF326573) |
| 1811) | DV_t4_strain_814669(AF326573)_17mers_1MM.txt | DV t4 strain 814669 (AF326573) |
| 1812) | DV_t4_strain_814669(AF326573)_17mers_2MM.txt | DV t4 strain 814669 (AF326573) |
| 1813) | DV_t4_strain_814669(AF326573)_18mers_0MM.txt | DV t4 strain 814669 (AF326573) |
| 1814) | DV_t4_strain_814669(AF326573)_18mers_1MM.txt | DV t4 strain 814669 (AF326573) |
| 1815) | DV_t4_strain_814669(AF326573)_18mers_2MM.txt | DV t4 strain 814669 (AF326573) |

Folder nmerseqsWNV - It contains 465 West Nile virus blind files, containing
sequences which are present in West Nile virus genomes but are not present in Human
genome even with two mismatches. The list below gives us the file name and the
corresponding genome name.

| | File Name | Genome Name |
|---|---|---|
| 1) | WN_1_19mers_0MM.txt | Kunjin virus gene for polyprotein (D00246) |
| 2) | WN_1_19mers_1MM.txt | Kunjin virus gene for polyprotein (D00246) |
| 3) | WN_1_19mers_2MM.txt | Kunjin virus gene for polyprotein (D00246) |
| 4) | WN_1_20mers_0MM.txt | Kunjin virus gene for polyprotein (D00246) |
| 5) | WN_1_20mers_1MM.txt | Kunjin virus gene for polyprotein (D00246) |
| 6) | WN_1_20mers_2MM.txt | Kunjin virus gene for polyprotein (D00246) |
| 7) | WN_1_21mers_0MM.txt | Kunjin virus gene for polyprotein (D00246) |
| 8) | WN_1_21mers_1MM.txt | Kunjin virus gene for polyprotein (D00246) |
| 9) | WN_1_22mers_0MM.txt | Kunjin virus gene for polyprotein (D00246) |
| 10) | WN_1_22mers_1MM.txt | Kunjin virus gene for polyprotein (D00246) |
| 11) | WN_1_22mers_2MM.txt | Kunjin virus gene for polyprotein (D00246) |
| 12) | WN_10_19mers_0MM.txt | WNV polyprotein precursor (AF533540) |
| 13) | WN_10_19mers_1MM.txt | WNV polyprotein precursor (AF533540) |
| 14) | WN_10_19mers_2MM.txt | WNV polyprotein precursor (AF533540) |
| 15) | WN_10_20mers_0MM.txt | WNV polyprotein precursor (AF533540) |
| 16) | WN_10_20mers_1MM.txt | WNV polyprotein precursor (AF533540) |
| 17) | WN_10_20mers_2MM.txt | WNV polyprotein precursor (AF533540) |
| 18) | WN_10_21mers_0MM.txt | WNV polyprotein precursor (AF533540) |
| 19) | WN_10_21mers_1MM.txt | WNV polyprotein precursor (AF533540) |
| 20) | WN_10_22mers_0MM.txt | WNV polyprotein precursor (AF533540) |
| 21) | WN_10_22mers_1MM.txt | WNV polyprotein precursor (AF533540) |
| 22) | WN_10_22mers_2MM.txt | WNV polyprotein precursor (AF533540) |
| 23) | WN_11_19mers_0MM.txt | WNV isolate KN3829 polyprotein gene (AY262283) |
| 24) | WN_11_19mers_1MM.txt | WNV isolate KN3829 polyprotein gene (AY262283) |
| 25) | WN_11_19mers_2MM.txt | WNV isolate KN3829 polyprotein gene (AY262283) |
| 26) | WN_11_20mers_0MM.txt | WNV isolate KN3829 polyprotein gene (AY262283) |
| 27) | WN_11_20mers_1MM.txt | WNV isolate KN3829 polyprotein gene (AY262283) |
| 28) | WN_11_20mers_2MM.txt | WNV isolate KN3829 polyprotein gene (AY262283) |
| 29) | WN_11_21mers_0MM.txt | WNV isolate KN3829 polyprotein gene (AY262283) |
| 30) | WN_11_21mers_1MM.txt | WNV isolate KN3829 polyprotein gene (AY262283) |
| 31) | WN_11_22mers_0MM.txt | WNV isolate KN3829 polyprotein gene (AY262283) |
| 32) | WN_11_22mers_1MM.txt | WNV isolate KN3829 polyprotein gene (AY262283) |
| 33) | WN_11_22mers_2MM.txt | WNV isolate KN3829 polyprotein gene (AY262283) |
| 34) | WN_12_19mers_0MM.txt | WNV isolate LEIV-Krnd88-190 (AY277251) |
| 35) | WN_12_19mers_1MM.txt | WNV isolate LEIV-Krnd88-190 (AY277251) |
| 36) | WN_12_19mers_2MM.txt | WNV isolate LEIV-Krnd88-190 (AY277251) |
| 37) | WN_12_20mers_0MM.txt | WNV isolate LEIV-Krnd88-190 (AY277251) |
| 38) | WN_12_20mers_1MM.txt | WNV isolate LEIV-Krnd88-190 (AY277251) |
| 39) | WN_12_20mers_2MM.txt | WNV isolate LEIV-Krnd88-190 (AY277251) |
| 40) | WN_12_21mers_0MM.txt | WNV isolate LEIV-Krnd88-190 (AY277251) |
| 41) | WN_12_21mers_1MM.txt | WNV isolate LEIV-Krnd88-190 (AY277251) |
| 42) | WN_12_22mers_0MM.txt | WNV isolate LEIV-Krnd88-190 (AY277251) |
| 43) | WN_12_22mers_1MM.txt | WNV isolate LEIV-Krnd88-190 (AY277251) |
| 44) | WN_12_22mers_2MM.txt | WNV isolate LEIV-Krnd88-190 (AY277251) |
| 45) | WN_13_19mers_0MM.txt | WNV isolate LEIV-Vlg99-27889 (AY27752) |
| 46) | WN_13_19mers_1MM.txt | WNV isolate LEIV-Vlg99-27889 (AY27752) |
| 47) | WN_13_19mers_2MM.txt | WNV isolate LEIV-Vlg99-27889 (AY27752) |
| 48) | WN_13_20mers_0MM.txt | WNV isolate LEIV-Vlg99-27889 (AY27752) |
| 49) | WN_13_20mers_1MM.txt | WNV isolate LEIV-Vlg99-27889 (AY27752) |
| 50) | WN_13_20mers_2MM.txt | WNV isolate LEIV-Vlg99-27889 (AY27752) |
| 51) | WN_13_21mers_0MM.txt | WNV isolate LEIV-Vlg99-27889 (AY27752) |
| 52) | WN_13_21mers_1MM.txt | WNV isolate LEIV-Vlg99-27889 (AY27752) |
| 53) | WN_13_22mers_0MM.txt | WNV isolate LEIV-Vlg99-27889 (AY27752) |
| 54) | WN_13_22mers_1MM.txt | WNV isolate LEIV-Vlg99-27889 (AY27752) |
| 55) | WN_13_22mers_2MM.txt | WNV isolate LEIV-Vlg99-27889 (AY27752) |
| 56) | WN_14_19mers_0MM.txt | WNV isolate Ast99-901 (AY278441) |
| 57) | WN_14_19mers_1MM.txt | WNV isolate Ast99-901 (AY278441) |
| 58) | WN_14_19mers_2MM.txt | WNV isolate Ast99-901 (AY278441) |
| 59) | WN_14_20mers_0MM.txt | WNV isolate Ast99-901 (AY278441) |
| 60) | WN_14_20mers_1MM.txt | WNV isolate Ast99-901 (AY278441) |
| 61) | WN_14_20mers_2MM.txt | WNV isolate Ast99-901 (AY278441) |
| 62) | WN_14_21mers_0MM.txt | WNV isolate Ast99-901 (AY278441) |
| 63) | WN_14_21mers_1MM.txt | WNV isolate Ast99-901 (AY278441) |
| 64) | WN_14_22mers_0MM.txt | WNV isolate Ast99-901 (AY278441) |
| 65) | WN_14_22mers_1MM.txt | WNV isolate Ast99-901 (AY278441) |
| 66) | WN_14_22mers_2MM.txt | WNV isolate Ast99-901 (AY278441) |
| 67) | WN_15_19mers_0MM.txt | WNV isolate LEIV-Vlg00-27924 (AY278442) |
| 68) | WN_15_19mers_1MM.txt | WNV isolate LEIV-Vlg00-27924 (AY278442) |
| 69) | WN_15_19mers_2MM.txt | WNV isolate LEIV-Vlg00-27924 (AY278442) |
| 70) | WN_15_20mers_0MM.txt | WNV isolate LEIV-Vlg00-27924 (AY278442) |
| 71) | WN_15_20mers_1MM.txt | WNV isolate LEIV-Vlg00-27924 (AY278442) |
| 72) | WN_15_20mers_2MM.txt | WNV isolate LEIV-Vlg00-27924 (AY278442) |
| 73) | WN_15_21mers_0MM.txt | WNV isolate LEIV-Vlg00-27924 (AY278442) |
| 74) | WN_15_21mers_1MM.txt | WNV isolate LEIV-Vlg00-27924 (AY278442) |
| 75) | WN_15_22mers_0MM.txt | WNV isolate LEIV-Vlg00-27924 (AY278442) |
| 76) | WN_15_22mers_1MM.txt | WNV isolate LEIV-Vlg00-27924 (AY278442) |
| 77) | WN_15_22mers_2MM.txt | WNV isolate LEIV-Vlg00-27924 (AY278442) |
| 78) | WN_16_19mers_0MM.txt | Kunjin virus clone FLSDX polyprotein mRNA (AY274504) |
| 79) | WN_16_19mers_1MM.txt | Kunjin virus clone FLSDX polyprotein mRNA (AY274504) |
| 80) | WN_16_19mers_2MM.txt | Kunjin virus clone FLSDX polyprotein mRNA (AY274504) |
| 81) | WN_16_20mers_0MM.txt | Kunjin virus clone FLSDX polyprotein mRNA (AY274504) |
| 82) | WN_16_20mers_1MM.txt | Kunjin virus clone FLSDX polyprotein mRNA (AY274504) |
| 83) | WN_16_20mers_2MM.txt | Kunjin virus clone FLSDX polyprotein mRNA (AY274504) |
| 84) | WN_16_21mers_0MM.txt | Kunjin virus clone FLSDX polyprotein mRNA (AY274504) |
| 85) | WN_16_21mers_1MM.txt | Kunjin virus clone FLSDX polyprotein mRNA (AY274504) |
| 86) | WN_16_22mers_0MM.txt | Kunjin virus clone FLSDX polyprotein mRNA (AY274504) |
| 87) | WN_16_22mers_1MM.txt | Kunjin virus clone FLSDX polyprotein mRNA (AY274504) |
| 88) | WN_16_22mers_2MM.txt | Kunjin virus clone FLSDX polyprotein mRNA (AY274504) |
| 89) | WN_17_19mers_0MM.txt | Kunjin virus clone pAKUN polyprotein mRNA (AY274505) |
| 90) | WN_17_19mers_1MM.txt | Kunjin virus clone pAKUN polyprotein mRNA (AY274505) |
| 91) | WN_17_19mers_2MM.txt | Kunjin virus clone pAKUN polyprotein mRNA (AY274505) |
| 92) | WN_17_20mers_0MM.txt | Kunjin virus clone pAKUN polyprotein mRNA (AY274505) |
| 93) | WN_17_20mers_1MM.txt | Kunjin virus clone pAKUN polyprotein mRNA (AY274505) |
| 94) | WN_17_20mers_2MM.txt | Kunjin virus clone pAKUN polyprotein mRNA (AY274505) |
| 95) | WN_17_21mers_0MM.txt | Kunjin virus clone pAKUN polyprotein mRNA (AY274505) |
| 96) | WN_17_21mers_1MM.txt | Kunjin virus clone pAKUN polyprotein mRNA (AY274505) |
| 97) | WN_17_22mers_0MM.txt | Kunjin virus clone pAKUN polyprotein mRNA (AY274505) |
| 98) | WN_17_22mers_1MM.txt | Kunjin virus clone pAKUN polyprotein mRNA (AY274505) |
| 99) | WN_17_22mers_2MM.txt | Kunjin virus clone pAKUN polyprotein mRNA (AY274505) |
| 100) | WN_18_19mers_0MM.txt | WNV isolate 2741 (AF206518) |
| 101) | WN_18_19mers_1MM.txt | WNV isolate 2741 (AF206518) |
| 102) | WN_18_19mers_2MM.txt | WNV isolate 2741 (AF206518) |
| 103) | WN_18_20mers_0MM.txt | WNV isolate 2741 (AF206518) |
| 104) | WN_18_20mers_1MM.txt | WNV isolate 2741 (AF206518) |
| 105) | WN_18_20mers_2MM.txt | WNV isolate 2741 (AF206518) |
| 106) | WN_18_21mers_0MM.txt | WNV isolate 2741 (AF206518) |
| 107) | WN_18_21mers_1MM.txt | WNV isolate 2741 (AF206518) |
| 108) | WN_18_22mers_0MM.txt | WNV isolate 2741 (AF206518) |
| 109) | WN_18_22mers_1MM.txt | WNV isolate 2741 (AF206518) |
| 110) | WN_18_22mers_2MM.txt | WNV isolate 2741 (AF206518) |
| 111) | WN_19_19mers_0MM.txt | WNV strain NY99-eqhs (AF260967) |
| 112) | WN_19_19mers_1MM.txt | WNV strain NY99-eqhs (AF260967) |
| 113) | WN_19_19mers_2MM.txt | WNV strain NY99-eqhs (AF260967) |
| 114) | WN_19_20mers_0MM.txt | WNV strain NY99-eqhs (AF260967) |
| 115) | WN_19_20mers_1MM.txt | WNV strain NY99-eqhs (AF260967) |
| 116) | WN_19_20mers_2MM.txt | WNV strain NY99-eqhs (AF260967) |
| 117) | WN_19_21mers_0MM.txt | WNV strain NY99-eqhs (AF260967) |
| 118) | WN_19_21mers_1MM.txt | WNV strain NY99-eqhs (AF260967) |
| 119) | WN_19_22mers_0MM.txt | WNV strain NY99-eqhs (AF260967) |
| 120) | WN_19_22mers_1MM.txt | WNV strain NY99-eqhs (AF260967) |
| 121) | WN_19_22mers_2MM.txt | WNV strain NY99-eqhs (AF260967) |
| 122) | WN_2_19mers_0MM.txt | WNV strain HNY1999 polyprotein (AF202541) |
| 123) | WN_2_19mers_1MM.txt | WNV strain HNY1999 polyprotein (AF202541) |
| 124) | WN_2_19mers_2MM.txt | WNV strain HNY1999 polyprotein (AF202541) |
| 125) | WN_2_20mers_0MM.txt | WNV strain HNY1999 polyprotein (AF202541) |
| 126) | WN_2_20mers_1MM.txt | WNV strain HNY1999 polyprotein (AF202541) |
| 127) | WN_2_20mers_2MM.txt | WNV strain HNY1999 polyprotein (AF202541) |
| 128) | WN_2_21mers_0MM.txt | WNV strain HNY1999 polyprotein (AF202541) |
| 129) | WN_2_21mers_1MM.txt | WNV strain HNY1999 polyprotein (AF202541) |
| 130) | WN_2_22mers_0MM.txt | WNV strain HNY1999 polyprotein (AF202541) |
| 131) | WN_2_22mers_1MM.txt | WNV strain HNY1999 polyprotein (AF202541) |
| 132) | WN_2_22mers_2MM.txt | WNV strain HNY1999 polyprotein (AF202541) |
| 133) | WN_20_20mers_0MM.txt | WNV strain Eg101 (AF260968) |
| 134) | WN_20_20mers_1MM.txt | WNV strain Eg101 (AF260968) |
| 135) | WN_20_20mers_2MM.txt | WNV strain Eg101 (AF260968) |
| 136) | WN_20_22mers_0MM.txt | WNV strain Eg101 (AF260968) |
| 137) | WN_20_22mers_1MM.txt | WNV strain Eg101 (AF260968) |
| 138) | WN_20_22mers_2MM.txt | WNV strain Eg101 (AF260968) |
| 139) | WN_21_19mers_0MM.txt | WNV strain Eg101 (AF260968) |
| 140) | WN_21_19mers_1MM.txt | WNV strain Eg101 (AF260968) |
| 141) | WN_21_19mers_2MM.txt | WNV strain Eg101 (AF260968) |
| 142) | WN_21_21mers_0MM.txt | WNV strain Eg101 (AF260968) |
| 143) | WN_21_21mers_1MM.txt | WNV strain Eg101 (AF260968) |
| 144) | WN_21_22mers_0MM.txt | WNV strain Eg101 (AF260968) |
| 145) | WN_21_22mers_1MM.txt | WNV strain Eg101 (AF260968) |
| 146) | WN_21_22mers_2MM.txt | WNV strain Eg101 (AF260968) |
| 147) | WN_22_19mers_0MM.txt | WNV strain RO97-50 (AF260969) |
| 148) | WN_22_19mers_1MM.txt | WNV strain RO97-50 (AF260969) |
| 149) | WN_22_19mers_2MM.txt | WNV strain RO97-50 (AF260969) |
| 150) | WN_22_21mers_0MM.txt | WNV strain RO97-50 (AF260969) |
| 151) | WN_22_21mers_1MM.txt | WNV strain RO97-50 (AF260969) |
| 152) | WN_22_22mers_0MM.txt | WNV strain RO97-50 (AF260969) |
| 153) | WN_22_22mers_1MM.txt | WNV strain RO97-50 (AF260969) |
| 154) | WN_22_22mers_2MM.txt | WNV strain RO97-50 (AF260969) |
| 155) | WN_23_19mers_0MM.txt | WNV strain NY99-flamingo382-99 (AF196835) |
| 156) | WN_23_19mers_1MM.txt | WNV strain NY99-flamingo382-99 (AF196835) |
| 157) | WN_23_19mers_2MM.txt | WNV strain NY99-flamingo382-99 (AF196835) |
| 158) | WN_23_21mers_0MM.txt | WNV strain NY99-flamingo382-99 (AF196835) |
| 159) | WN_23_21mers_1MM.txt | WNV strain NY99-flamingo382-99 (AF196835) |
| 160) | WN_23_22mers_0MM.txt | WNV strain NY99-flamingo382-99 (AF196835) |
| 161) | WN_23_22mers_1MM.txt | WNV strain NY99-flamingo382-99 (AF196835) |
| 162) | WN_23_22mers_2MM.txt | WNV strain NY99-flamingo382-99 (AF196835) |
| 163) | WN_24_19mers_0MM.txt | WNV strain PaAn001 polyprotein (pol) gene (AY268132) |
| 164) | WN_24_19mers_1MM.txt | WNV strain PaAn001 polyprotein (pol) gene (AY268132) |
| 165) | WN_24_19mers_2MM.txt | WNV strain PaAn001 polyprotein (pol) gene (AY268132) |
| 166) | WN_24_20mers_0MM.txt | WNV strain PaAn001 polyprotein (pol) gene (AY268132) |
| 167) | WN_24_20mers_1MM.txt | WNV strain PaAn001 polyprotein (pol) gene (AY268132) |
| 168) | WN_24_20mers_2MM.txt | WNV strain PaAn001 polyprotein (pol) gene (AY268132) |
| 169) | WN_24_21mers_0MM.txt | WNV strain PaAn001 polyprotein (pol) gene (AY268132) |
| 170) | WN_24_21mers_1MM.txt | WNV strain PaAn001 polyprotein (pol) gene (AY268132) |
| 171) | WN_24_22mers_0MM.txt | WNV strain PaAn001 polyprotein (pol) gene (AY268132) |
| 172) | WN_24_22mers_1MM.txt | WNV strain PaAn001 polyprotein (pol) gene (AY268132) |
| 173) | WN_24_22mers_2MM.txt | WNV strain PaAn001 polyprotein (pol) gene (AY268132) |
| 174) | WN_25_19mers_0MM.txt | WNV strain Chin-01 (AY490240) |
| 175) | WN_25_19mers_1MM.txt | WNV strain Chin-01 (AY490240) |
| 176) | WN_25_19mers_2MM.txt | WNV strain Chin-01 (AY490240) |
| 177) | WN_25_20mers_0MM.txt | WNV strain Chin-01 (AY490240) |
| 178) | WN_25_20mers_1MM.txt | WNV strain Chin-01 (AY490240) |
| 179) | WN_25_20mers_2MM.txt | WNV strain Chin-01 (AY490240) |
| 180) | WN_25_21mers_0MM.txt | WNV strain Chin-01 (AY490240) |
| 181) | WN_25_21mers_1MM.txt | WNV strain Chin-01 (AY490240) |
| 182) | WN_3_19mers_0MM.txt | WNV VLG-4 polyprotein precursor (AF317203) |
| 183) | WN_3_19mers_1MM.txt | WNV VLG-4 polyprotein precursor (AF317203) |
| 184) | WN_3_19mers_2MM.txt | WNV VLG-4 polyprotein precursor (AF317203) |
| 185) | WN_3_20mers_0MM.txt | WNV VLG-4 polyprotein precursor (AF317203) |
| 186) | WN_3_20mers_1MM.txt | WNV VLG-4 polyprotein precursor (AF317203) |
| 187) | WN_3_20mers_2MM.txt | WNV VLG-4 polyprotein precursor (AF317203) |
| 188) | WN_3_21mers_0MM.txt | WNV VLG-4 polyprotein precursor (AF317203) |
| 189) | WN_3_21mers_1MM.txt | WNV VLG-4 polyprotein precursor (AF317203) |
| 190) | WN_3_22mers_0MM.txt | WNV VLG-4 polyprotein precursor (AF317203) |
| 191) | WN_3_22mers_1MM.txt | WNV VLG-4 polyprotein precursor (AF317203) |
| 192) | WN_3_22mers_2MM.txt | WNV VLG-4 polyprotein precursor (AF317203) |
| 193) | WN_4_19mers_0MM.txt | WNV strain IS-98 STD (AF481864) |
| 194) | WN_4_19mers_1MM.txt | WNV strain IS-98 STD (AF481864) |
| 195) | WN_4_19mers_2MM.txt | WNV strain IS-98 STD (AF481864) |
| 196) | WN_4_20mers_0MM.txt | WNV strain IS-98 STD (AF481864) |
| 197) | WN_4_20mers_1MM.txt | WNV strain IS-98 STD (AF481864) |
| 198) | WN_4_20mers_2MM.txt | WNV strain IS-98 STD (AF481864) |
| 199) | WN_4_21mers_0MM.txt | WNV strain IS-98 STD (AF481864) |
| 200) | WN_4_21mers_1MM.txt | WNV strain IS-98 STD (AF481864) |
| 201) | WN_4_22mers_0MM.txt | WNV strain IS-98 STD (AF481864) |
| 202) | WN_4_22mers_1MM.txt | WNV strain IS-98 STD (AF481864) |
| 203) | WN_4_22mers_2MM.txt | WNV strain IS-98 STD (AF481864) |
| 204) | WN_5_19mers_0MM.txt | WNV isolate WN MD 2000-crow265 (AF404753) |
| 205) | WN_5_19mers_1MM.txt | WNV isolate WN MD 2000-crow265 (AF404753) |
| 206) | WN_5_19mers_2MM.txt | WNV isolate WN MD 2000-crow265 (AF404753) |
| 207) | WN_5_20mers_0MM.txt | WNV isolate WN MD 2000-crow265 (AF404753) |
| 208) | WN_5_20mers_1MM.txt | WNV isolate WN MD 2000-crow265 (AF404753) |
| 209) | WN_5_20mers_2MM.txt | WNV isolate WN MD 2000-crow265 (AF404753) |
| 210) | WN_5_21mers_0MM.txt | WNV isolate WN MD 2000-crow265 (AF404753) |
| 211) | WN_5_21mers_1MM.txt | WNV isolate WN MD 2000-crow265 (AF404753) |
| 212) | WN_5_22mers_0MM.txt | WNV isolate WN MD 2000-crow265 (AF404753) |
| 213) | WN_5_22mers_1MM.txt | WNV isolate WN MD 2000-crow265 (AF404753) |
| 214) | WN_5_22mers_2MM.txt | WNV isolate WN MD 2000-crow265 (AF404753) |
| 215) | WN_6_19mers_0MM.txt | WNV isolate WN NJ 2000 MQ5488 (AF404754) |
| 216) | WN_6_19mers_1MM.txt | WNV isolate WN NJ 2000 MQ5488 (AF404754) |
| 217) | WN_6_19mers_2MM.txt | WNV isolate WN NJ 2000 MQ5488 (AF404754) |
| 218) | WN_6_20mers_0MM.txt | WNV isolate WN NJ 2000 MQ5488 (AF404754) |
| 219) | WN_6_20mers_1MM.txt | WNV isolate WN NJ 2000 MQ5488 (AF404754) |
| 220) | WN_6_20mers_2MM.txt | WNV isolate WN NJ 2000 MQ5488 (AF404754) |
| 221) | WN_6_21mers_0MM.txt | WNV isolate WN NJ 2000 MQ5488 (AF404754) |
| 222) | WN_6_21mers_1MM.txt | WNV isolate WN NJ 2000 MQ5488 (AF404754) |
| 223) | WN_6_22mers_0MM.txt | WNV isolate WN NJ 2000 MQ5488 (AF404754) |
| 224) | WN_6_22mers_1MM.txt | WNV isolate WN NJ 2000 MQ5488 (AF404754) |
| 225) | WN_6_22mers_2MM.txt | WNV isolate WN NJ 2000 MQ5488 (AF404754) |
| 226) | WN_7_19mers_0MM.txt | WNV isolate WN NY 2000-grouse3282 (AF404755) |
| 227) | WN_7_19mers_1MM.txt | WNV isolate WN NY 2000-grouse3282 (AF404755) |
| 228) | WN_7_19mers_2MM.txt | WNV isolate WN NY 2000-grouse3282 (AF404755) |
| 229) | WN_7_20mers_0MM.txt | WNV isolate WN NY 2000-grouse3282 (AF404755) |
| 230) | WN_7_20mers_1MM.txt | WNV isolate WN NY 2000-grouse3282 (AF404755) |
| 231) | WN_7_20mers_2MM.txt | WNV isolate WN NY 2000-grouse3282 (AF404755) |
| 232) | WN_7_21mers_0MM.txt | WNV isolate WN NY 2000-grouse3282 (AF404755) |
| 233) | WN_7_21mers_1MM.txt | WNV isolate WN NY 2000-grouse3282 (AF404755) |
| 234) | WN_7_22mers_0MM.txt | WNV isolate WN NY 2000-grouse3282 (AF404755) |
| 235) | WN_7_22mers_1MM.txt | WNV isolate WN NY 2000-grouse3282 (AF404755) |
| 236) | WN_7_22mers_2MM.txt | WNV isolate WN NY 2000-grouse3282 (AF404755) |
| 237) | WN_8_19mers_0MM.txt | WNV isolate WN NY 2000-crow3356 (AF404756) |
| 238) | WN_8_19mers_1MM.txt | WNV isolate WN NY 2000-crow3356 (AF404756) |
| 239) | WN_8_19mers_2MM.txt | WNV isolate WN NY 2000-crow3356 (AF404756) |
| 240) | WN_8_20mers_0MM.txt | WNV isolate WN NY 2000-crow3356 (AF404756) |
| 241) | WN_8_20mers_1MM.txt | WNV isolate WN NY 2000-crow3356 (AF404756) |
| 242) | WN_8_20mers_2MM.txt | WNV isolate WN NY 2000-crow3356 (AF404756) |
| 243) | WN_8_21mers_0MM.txt | WNV isolate WN NY 2000-crow3356 (AF404756) |
| 244) | WN_8_21mers_1MM.txt | WNV isolate WN NY 2000-crow3356 (AF404756) |
| 245) | WN_8_22mers_0MM.txt | WNV isolate WN NY 2000-crow3356 (AF404756) |
| 246) | WN_8_22mers_1MM.txt | WNV isolate WN NY 2000-crow3356 (AF404756) |
| 247) | WN_8_22mers_2MM.txt | WNV isolate WN NY 2000-crow3356 (AF404756) |
| 248) | WN_9_19mers_0MM.txt | WNV isolate WN Italy 1998-equine (AF404757) |
| 249) | WN_9_19mers_1MM.txt | WNV isolate WN Italy 1998-equine (AF404757) |
| 250) | WN_9_19mers_2MM.txt | WNV isolate WN Italy 1998-equine (AF404757) |
| 251) | WN_9_20mers_0MM.txt | WNV isolate WN Italy 1998-equine (AF404757) |
| 252) | WN_9_20mers_1MM.txt | WNV isolate WN Italy 1998-equine (AF404757) |
| 253) | WN_9_20mers_2MM.txt | WNV isolate WN Italy 1998-equine (AF404757) |
| 254) | WN_9_21mers_0MM.txt | WNV isolate WN Italy 1998-equine (AF404757) |
| 255) | WN_9_21mers_1MM.txt | WNV isolate WN Italy 1998-equine (AF404757) |
| 256) | WN_9_22mers_0MM.txt | WNV isolate WN Italy 1998-equine (AF404757) |
| 257) | WN_9_22mers_1MM.txt | WNV isolate WN Italy 1998-equine (AF404757) |
| 258) | WN_9_22mers_2MM.txt | WNV isolate WN Italy 1998-equine (AF404757) |
| 259) | WNV(NC_001563)_16mers_0MM.txt | WNV(NC 001563) |
| 260) | WNV(NC_001563)_16mers_1MM.txt | WNV(NC 001563) |
| 261) | WNV(NC_001563)_16mers_2MM.txt | WNV(NC 001563) |
| 262) | WNV(NC_001563)_17mers_0MM.txt | WNV(NC 001563) |
| 263) | WNV(NC_001563)_17mers_1MM.txt | WNV(NC 001563) |
| 264) | WNV(NC_001563)_17mers_2MM.txt | WNV(NC 001563) |
| 265) | WNV(NC_001563)_18mers_0MM.txt | WNV(NC 001563) |
| 266) | WNV(NC_001563)_18mers_1MM.txt | WNV(NC 001563) |
| 267) | WNV(NC_001563)_18mers_2MM.txt | WNV(NC 001563) |
| 268) | WNV_isolate_2741(AF206518)_16mers_0MM.txt | WNV isolate 2741(AF206518) |
| 269) | WNV_isolate_2741(AF206518)_16mers_1MM.txt | WNV isolate 2741(AF206518) |
| 270) | WNV_isolate_2741(AF206518)_16mers_2MM.txt | WNV isolate 2741(AF206518) |
| 271) | WNV_isolate_2741(AF206518)_17mers_0MM.txt | WNV isolate 2741(AF206518) |
| 272) | WNV_isolate_2741(AF206518)_17mers_1MM.txt | WNV isolate 2741(AF206518) |
| 273) | WNV_isolate_2741(AF206518)_17mers_2MM.txt | WNV isolate 2741(AF206518) |
| 274) | WNV_isolate_2741(AF206518)_18mers_0MM.txt | WNV isolate 2741(AF206518) |
| 275) | WNV_isolate_2741(AF206518)_18mers_1MM.txt | WNV isolate 2741(AF206518) |
| 276) | WNV_isolate_2741(AF206518)_18mers_2MM.txt | WNV isolate 2741(AF206518) |
| 277) | WNV_isolate_Ast99-901(AY278441)_16mers_0MM.txt | WNV isolate Ast99-901(AY278441) |
| 278) | WNV_isolate_Ast99-901(AY278441)_16mers_1MM.txt | WNV isolate Ast99-901(AY278441) |
| 279) | WNV_isolate_Ast99-901(AY278441)_16mers_2MM.txt | WNV isolate Ast99-901(AY278441) |
| 280) | WNV_isolate_Ast99-901(AY278441)_17mers_0MM.txt | WNV isolate Ast99-901(AY278441) |
| 281) | WNV_isolate_Ast99-901(AY278441)_17mers_1MM.txt | WNV isolate Ast99-901(AY278441) |
| 282) | WNV_isolate_Ast99-901(AY278441)_17mers_2MM.txt | WNV isolate Ast99-901(AY278441) |
| 283) | WNV_isolate_Ast99-901(AY278441)_18mers_0MM.txt | WNV isolate Ast99-901(AY278441) |
| 284) | WNV_isolate_Ast99-901(AY278441)_18mers_1MM.txt | WNV isolate Ast99-901(AY278441) |
| 285) | WNV_isolate_Ast99-901(AY278441)_18mers_2MM.txt | WNV isolate Ast99-901(AY278441) |
| 286) | WNV_isolate_KN3829_polyprotein_gene(AY262283)_16mers_0MM.txt | WNV isolate KN3829 |
| | | polyprotein gene(AY262283) |
| 287) | WNV_isolate_KN3829_polyprotein_gene(AY262283)_16mers_1MM.txt | WNV isolate KN3829 |
| | | polyprotein gene(AY262283) |
| 288) | WNV_isolate_KN3829_polyprotein_gene(AY262283)_16mers_2MM.txt | WNV isolate KN3829 |
| | | polyprotein gene(AY262283) |
| 289) | WNV_isolate_KN3829_polyprotein_gene(AY262283)_17mers_0MM.txt | WNV isolate KN3829 |
| | | polyprotein gene(AY262283) |
| 290) | WNV_isolate_KN3829_polyprotein_gene(AY262283)_17mers_1MM.txt | WNV isolate KN3829 |
| | | polyprotein gene(AY262283) |
| 291) | WNV_isolate_KN3829_polyprotein_gene(AY262283)_17mers_2MM.txt | WNV isolate KN3829 |
| | | polyprotein gene(AY262283) |
| 292) | WNV_isolate_KN3829_polyprotein_gene(AY262283)_18mers_0MM.txt | WNV isolate KN3829 |
| | | polyprotein gene(AY262283) |
| 293) | WNV_isolate_KN3829_polyprotein_gene(AY262283)_18mers_1MM.txt | WNV isolate KN3829 |
| | | polyprotein gene(AY262283) |
| 294) | WNV_isolate_KN3829_polyprotein_gene(AY262283)_18mers_2MM.txt | WNV isolate KN3829 |
| | | polyprotein gene(AY262283) |
| 295) | WNV_isolate_LEIV-Krnd88-190(AY277251)_16mers_0MM.txt | WNV isolate LEIV-Krnd88- |
| | | 190(AY277251) |
| 296) | WNV_isolate_LEIV-Krnd88-190(AY277251)_16mers_1MM.txt | WNV isolate LEIV-Krnd88- |
| | | 190(AY277251) |
| 297) | WNV_isolate_LEIV-Krnd88-190(AY277251)_16mers_2MM.txt | WNV isolate LEIV-Krnd88- |
| | | 190(AY277251) |
| 298) | WNV_isolate_LEIV-Krnd88-190(AY277251)_17mers_0MM.txt | WNV isolate LEIV-Krnd88- |
| | | 190(AY277251) |
| 299) | WNV_isolate_LEIV-Krnd88-190(AY277251)_17mers_1MM.txt | WNV isolate LEIV-Krnd88- |
| | | 190(AY277251) |
| 300) | WNV_isolate_LEIV-Krnd88-190(AY277251)_17mers_2MM.txt | WNV isolate LEIV-Krnd88- |
| | | 190(AY277251) |
| 301) | WNV_isolate_LEIV-Krnd88-190(AY277251)_18mers_0MM.txt | WNV isolate LEIV-Krnd88- |
| | | 190(AY277251) |
| 302) | WNV_isolate_LEIV-Krnd88-190(AY277251)_18mers_1MM.txt | WNV isolate LEIV-Krnd88- |
| | | 190(AY277251) |
| 303) | WNV_isolate_LEIV-Krnd88-190(AY277251)_18mers_2MM.txt | WNV isolate LEIV-Krnd88- |
| | | 190(AY277251) |
| 304) | WNV_isolate_LEIV-Vlg00-27924(AY278442)_16mers_0MM.txt | WNV isolate LEIV-Vlg00- |
| | | 27924(AY278442) |
| 305) | WNV_isolate_LEIV-Vlg00-27924(AY278442)_16mers_1MM.txt | WNV isolate LEIV-Vlg00- |
| | | 27924(AY278442) |
| 306) | WNV_isolate_LEIV-Vlg00-27924(AY278442)_16mers_2MM.txt | WNV isolate LEIV-Vlg00- |
| | | 27924(AY278442) |
| 307) | WNV_isolate_LEIV-Vlg00-27924(AY278442)_17mers_0MM.txt | WNV isolate LEIV-Vlg00- |
| | | 27924(AY278442) |
| 308) | WNV_isolate_LEIV-Vlg00-27924(AY278442)_17mers_1MM.txt | WNV isolate LEIV-Vlg00- |
| | | 27924(AY278442) |
| 309) | WNV_isolate_LEIV-Vlg00-27924(AY278442)_17mers_2MM.txt | WNV isolate LEIV-Vlg00- |
| | | 27924(AY278442) |
| 310) | WNV_isolate_LEIV-Vlg00-27924(AY278442)_18mers_0MM.txt | WNV isolate LEIV-Vlg00- |
| | | 27924(AY278442) |
| 311) | WNV_isolate_LEIV-Vlg00-27924(AY278442)_18mers_1MM.txt | WNV isolate LEIV-Vlg00- |
| | | 27924(AY278442) |
| 312) | WNV_isolate_LEIV-Vlg00-27924(AY278442)_18mers_2MM.txt | WNV isolate LEIV-Vlg00- |
| | | 27924(AY278442) |
| 313) | WNV_isolate_LEIV-Vlg99-27889(AY27752)_16mers_0MM.txt | WNV isolate LEIV-Vlg99- |
| | | 27889(AY27752) |
| 314) | WNV_isolate_LEIV-Vlg99-27889(AY27752)_16mers_1MM.txt | WNV isolate LEIV-Vlg99- |
| | | 27889(AY27752) |
| 315) | WNV_isolate_LEIV-Vlg99-27889(AY27752)_16mers_2MM.txt | WNV isolate LEIV-Vlg99- |
| | | 27889(AY27752) |
| 316) | WNV_isolate_LEIV-Vlg99-27889(AY27752)_17mers_0MM.txt | WNV isolate LEIV-Vlg99- |
| | | 27889(AY27752) |
| 317) | WNV_isolate_LEIV-Vlg99-27889(AY27752)_17mers_1MM.txt | WNV isolate LEIV-Vlg99- |
| | | 27889(AY27752) |
| 318) | WNV_isolate_LEIV-Vlg99-27889(AY27752)_17mers_2MM.txt | WNV isolate LEIV-Vlg99- |
| | | 27889(AY27752) |
| 319) | WNV_isolate_LEIV-Vlg99-27889(AY27752)_18mers_0MM.txt | WNV isolate LEIV-Vlg99- |
| | | 27889(AY27752) |
| 320) | WNV_isolate_LEIV-Vlg99-27889(AY27752)_18mers_1MM.txt | WNV isolate LEIV-Vlg99- |
| | | 27889(AY27752) |
| 321) | WNV_isolate_LEIV-Vlg99-27889(AY27752)_18mers_2MM.txt | WNV isolate LEIV-Vlg99- |
| | | 27889(AY27752) |
| 322) | WNV_isolate_WN_Italy_1998-equine(AF404757)_16mers_0MM.txt | WNV isolate WN Italy |
| | | 1998-equine(AF404757) |
| 323) | WNV_isolate_WN_Italy_1998-equine(AF404757)_16mers_1MM.txt | WNV isolate WN Italy |
| | | 1998-equine(AF404757) |
| 324) | WNV_isolate_WN_Italy_1998-equine(AF404757)_16mers_2MM.txt | WNV isolate WN Italy |
| | | 1998-equine(AF404757) |
| 325) | WNV_isolate_WN_Italy_1998-equine(AF404757)_17mers_0MM.txt | WNV isolate WN Italy |
| | | 1998-equine(AF404757) |
| 326) | WNV_isolate_WN_Italy_1998-equine(AF404757)_17mers_1MM.txt | WNV isolate WN Italy |
| | | 1998-equine(AF404757) |
| 327) | WNV_isolate_WN_Italy_1998-equine(AF404757)_17mers_2MM.txt | WNV isolate WN Italy |
| | | 1998-equine(AF404757) |
| 328) | WNV_isolate_WN_Italy_1998-equine(AF404757)_18mers_0MM.txt | WNV isolate WN Italy |
| | | 1998-equine(AF404757) |
| 329) | WNV_isolate_WN_Italy_1998-equine(AF404757)_18mers_1MM.txt | WNV isolate WN Italy |
| | | 1998-equine(AF404757) |
| 330) | WNV_isolate_WN_Italy_1998-equine(AF404757)_18mers_2MM.txt | WNV isolate WN Italy |
| | | 1998-equine(AF404757) |
| 331) | WNV_isolate_WN_MD_2000-crow265(AF404753)_16mers_0MM.txt | WNV isolate WN MD |
| | | 2000-crow265(AF404753) |
| 332) | WNV_isolate_WN_MD_2000-crow265(AF404753)_16mers_1MM.txt | WNV isolate WN MD |
| | | 2000-crow265(AF404753) |
| 333) | WNV_isolate_WN_MD_2000-crow265(AF404753)_16mers_2MM.txt | WNV isolate WN MD |
| | | 2000-crow265(AF404753) |
| 334) | WNV_isolate_WN_MD_2000-crow265(AF404753)_17mers_0MM.txt | WNV isolate WN MD |
| | | 2000-crow265(AF404753) |
| 335) | WNV_isolate_WN_MD_2000-crow265(AF404753)_17mers_1MM.txt | WNV isolate WN MD |
| | | 2000-crow265(AF404753) |
| 336) | WNV_isolate_WN_MD_2000-crow265(AF404753)_17mers_2MM.txt | WNV isolate WN MD |
| | | 2000-crow265(AF404753) |
| 337) | WNV_isolate_WN_MD_2000-crow265(AF404753)_18mers_0MM.txt | WNV isolate WN MD |
| | | 2000-crow265(AF404753) |
| 338) | WNV_isolate_WN_MD_2000-crow265(AF404753)_18mers_1MM.txt | WNV isolate WN MD |
| | | 2000-crow265(AF404753) |
| 339) | WNV_isolate_WN_MD_2000-crow265(AF404753)_18mers_2MM.txt | WNV isolate WN MD |
| | | 2000-crow265(AF404753) |
| 340) | WNV_isolate_WN_NJ_2000_MQ5488(AF404754)_16mers_0MM.txt | WNV isolate WN NJ 2000 |
| | | MQ5488(AF404754) |
| 341) | WNV_isolate_WN_NJ_2000_MQ5488(AF404754)_16mers_1MM.txt | WNV isolate WN NJ 2000 |
| | | MQ5488(AF404754) |
| 342) | WNV_isolate_WN_NJ_2000_MQ5488(AF404754)_16mers_2MM.txt | WNV isolate WN NJ 2000 |
| | | MQ5488(AF404754) |
| 343) | WNV_isolate_WN_NJ_2000_MQ5488(AF404754)_17mers_0MM.txt | WNV isolate WN NJ 2000 |
| | | MQ5488(AF404754) |
| 344) | WNV_isolate_WN_NJ_2000_MQ5488(AF404754)_17mers_1MM.txt | WNV isolate WN NJ 2000 |
| | | MQ5488(AF404754) |
| 345) | WNV_isolate_WN_NJ_2000_MQ5488(AF404754)_17mers_2MM.txt | WNV isolate WN NJ 2000 |
| | | MQ5488(AF404754) |
| 346) | WNV_isolate_WN_NJ_2000_MQ5488(AF404754)_18mers_0MM.txt | WNV isolate WN NJ 2000 |
| | | MQ5488(AF404754) |
| 347) | WNV_isolate_WN_NJ_2000_MQ5488(AF404754)_18mers_1MM.txt | WNV isolate WN NJ 2000 |
| | | MQ5488(AF404754) |
| 348) | WNV_isolate_WN_NJ_2000_MQ5488(AF404754)_18mers_2MM.txt | WNV isolate WN NJ 2000 |
| | | MQ5488(AF404754) |
| 349) | WNV_isolate_WN_NY_2000-crow3356(AF404756)_16mers_0MM.txt | WNV isolate WN NY |
| | | 2000-crow3356(AF404756) |
| 350) | WNV_isolate_WN_NY_2000-crow3356(AF404756)_16mers_1MM.txt | WNV isolate WN NY |
| | | 2000-crow3356(AF404756) |
| 351) | WNV_isolate_WN_NY_2000-crow3356(AF404756)_16mers_2MM.txt | WNV isolate WN NY |
| | | 2000-crow3356(AF404756) |
| 352) | WNV_isolate_WN_NY_2000-crow3356(AF404756)_17mers_0MM.txt | WNV isolate WN NY |
| | | 2000-crow3356(AF404756) |
| 353) | WNV_isolate_WN_NY_2000-crow3356(AF404756)_17mers_1MM.txt | WNV isolate WN NY |
| | | 2000-crow3356(AF404756) |
| 354) | WNV_isolate_WN_NY_2000-crow3356(AF404756)_17mers_2MM.txt | WNV isolate WN NY |
| | | 2000-crow3356(AF404756) |
| 355) | WNV_isolate_WN_NY_2000-crow3356(AF404756)_18mers_0MM.txt | WNV isolate WN NY |
| | | 2000-crow3356(AF404756) |
| 356) | WNV_isolate_WN_NY_2000-crow3356(AF404756)_18mers_1MM.txt | WNV isolate WN NY |
| | | 2000-crow3356(AF404756) |
| 357) | WNV_isolate_WN_NY_2000-crow3356(AF404756)_18mers_2MM.txt | WNV isolate WN NY |
| | | 2000-crow3356(AF404756) |
| 358) | WNV_isolate_WN_NY_2000-grouse3282(AF404755)_16mers_0MM.txt | WNV isolate WN NY |
| | | 2000-grouse3282(AF404755) |
| 359) | WNV_isolate_WN_NY_2000-grouse3282(AF404755)_16mers_1MM.txt | WNV isolate WN NY |
| | | 2000-grouse3282(AF404755) |
| 360) | WNV_isolate_WN_NY_2000-grouse3282(AF404755)_16mers_2MM.txt | WNV isolate WN NY |
| | | 2000-grouse3282(AF404755) |
| 361) | WNV_isolate_WN_NY_2000-grouse3282(AF404755)_17mers_0MM.txt | WNV isolate WN NY |
| | | 2000-grouse3282(AF404755) |
| 362) | WNV_isolate_WN_NY_2000-grouse3282(AF404755)_17mers_1MM.txt | WNV isolate WN NY |
| | | 2000-grouse3282(AF404755) |
| 363) | WNV_isolate_WN_NY_2000-grouse3282(AF404755)_17mers_2MM.txt | WNV isolate WN NY |
| | | 2000-grouse3282(AF404755) |
| 364) | WNV_isolate_WN_NY_2000-grouse3282(AF404755)_18mers_0MM.txt | WNV isolate WN NY |
| | | 2000-grouse3282(AF404755) |
| 365) | WNV_isolate_WN_NY_2000-grouse3282(AF404755)_18mers_1MM.txt | WNV isolate WN NY |
| | | 2000-grouse3282(AF404755) |
| 366) | WNV_isolate_WN_NY_2000-grouse3282(AF404755)_18mers_2MM.txt | WNV isolate WN NY |
| | | 2000-grouse3282(AF404755) |
| 367) | WNV_polyprotein_precursor(AF533540)_16mers_0MM.txt | WNV polyprotein |
| | | precursor(AF533540) |
| 368) | WNV_polyprotein_precursor(AF533540)_16mers_1MM.txt | WNV polyprotein |
| | | precursor(AF533540) |
| 369) | WNV_polyprotein_precursor(AF533540)_16mers_2MM.txt | WNV polyprotein |
| | | precursor(AF533540) |
| 370) | WNV_polyprotein_precursor(AF533540)_17mers_0MM.txt | WNV polyprotein |
| | | precursor(AF533540) |
| 371) | WNV_polyprotein_precursor(AF533540)_17mers_1MM.txt | WNV polyprotein |
| | | precursor(AF533540) |
| 372) | WNV_polyprotein_precursor(AF533540)_17mers_2MM.txt | WNV polyprotein |
| | | precursor(AF533540) |
| 373) | WNV_polyprotein_precursor(AF533540)_18mers_0MM.txt | WNV polyprotein |
| | | precursor(AF533540) |
| 374) | WNV_polyprotein_precursor(AF533540)_18mers_1MM.txt | WNV polyprotein |
| | | precursor(AF533540) |
| 375) | WNV_polyprotein_precursor(AF533540)_18mers_2MM.txt | WNV polyprotein |
| | | precursor(AF533540) |
| 376) | WNV_strain_Chin-01(AY490240)_16mers_0MM.txt | WNV strain Chin-01(AY490240) |
| 377) | WNV_strain_Chin-01(AY490240)_16mers_1MM.txt | WNV strain Chin-01(AY490240) |
| 378) | WNV_strain_Chin-01(AY490240)_16mers_2MM.txt | WNV strain Chin-01(AY490240) |
| 379) | WNV_strain_Chin-01(AY490240)_17mers_0MM.txt | WNV strain Chin-01(AY490240) |
| 380) | WNV_strain_Chin-01(AY490240)_17mers_1MM.txt | WNV strain Chin-01(AY490240) |
| 381) | WNV_strain_Chin-01(AY490240)_17mers_2MM.txt | WNV strain Chin-01(AY490240) |
| 382) | WNV_strain_Chin-01(AY490240)_18mers_0MM.txt | WNV strain Chin-01(AY490240) |
| 383) | WNV_strain_Chin-01(AY490240)_18mers_1MM.txt | WNV strain Chin-01(AY490240) |
| 384) | WNV_strain_Chin-01(AY490240)_18mers_2MM.txt | WNV strain Chin-01(AY490240) |
| 385) | WNV_strain_Eg101(AF260968)_16mers_0MM.txt | WNV strain Eg101(AF260968) |
| 386) | WNV_strain_Eg101(AF260968)_16mers_1MM.txt | WNV strain Eg101(AF260968) |
| 387) | WNV_strain_Eg101(AF260968)_16mers_2MM.txt | WNV strain Eg101(AF260968) |
| 388) | WNV_strain_Eg101(AF260968)_17mers_0MM.txt | WNV strain Eg101(AF260968) |
| 389) | WNV_strain_Eg101(AF260968)_17mers_1MM.txt | WNV strain Eg101(AF260968) |
| 390) | WNV_strain_Eg101(AF260968)_17mers_2MM.txt | WNV strain Eg101(AF260968) |
| 391) | WNV_strain_Eg101(AF260968)_18mers_0MM.txt | WNV strain Eg101(AF260968) |
| 392) | WNV_strain_Eg101(AF260968)_18mers_1MM.txt | WNV strain Eg101(AF260968) |
| 393) | WNV_strain_Eg101(AF260968)_18mers_2MM.txt | WNV strain Eg101(AF260968) |
| 394) | WNV_strain_HNY1999(AF202541)_16mers_0MM.txt | WNV strain HNY1999(AF202541) |
| 395) | WNV_strain_HNY1999(AF202541)_16mers_1MM.txt | WNV strain HNY1999(AF202541) |
| 396) | WNV_strain_HNY1999(AF202541)_16mers_2MM.txt | WNV strain HNY1999(AF202541) |
| 397) | WNV_strain_HNY1999(AF202541)_17mers_0MM.txt | WNV strain HNY1999(AF202541) |
| 398) | WNV_strain_HNY1999(AF202541)_17mers_1MM.txt | WNV strain HNY1999(AF202541) |
| 399) | WNV_strain_HNY1999(AF202541)_17mers_2MM.txt | WNV strain HNY1999(AF202541) |
| 400) | WNV_strain_HNY1999(AF202541)_18mers_0MM.txt | WNV strain HNY1999(AF202541) |
| 401) | WNV_strain_HNY1999(AF202541)_18mers_1MM.txt | WNV strain HNY1999(AF202541) |
| 402) | WNV_strain_HNY1999(AF202541)_18mers_2MM.txt | WNV strain HNY1999(AF202541) |
| 403) | WNV_strain_IS-98_STD(AF81864)_16mers_0MM.txt | WNV strain IS-98 STD(AF81864) |
| 404) | WNV_strain_IS-98_STD(AF81864)_16mers_1MM.txt | WNV strain IS-98 STD(AF81864) |
| 405) | WNV_strain_IS-98_STD(AF81864)_16mers_2MM.txt | WNV strain IS-98 STD(AF81864) |
| 406) | WNV_strain_IS-98_STD(AF81864)_17mers_0MM.txt | WNV strain IS-98 STD(AF81864) |
| 407) | WNV_strain_IS-98_STD(AF81864)_17mers_1MM.txt | WNV strain IS-98 STD(AF81864) |
| 408) | WNV_strain_IS-98_STD(AF81864)_17mers_2MM.txt | WNV strain IS-98 STD(AF81864) |
| 409) | WNV_strain_IS-98_STD(AF81864)_18mers_0MM.txt | WNV strain IS-98 STD(AF81864) |
| 410) | WNV_strain_IS-98_STD(AF81864)_18mers_1MM.txt | WNV strain IS-98 STD(AF81864) |
| 411) | WNV_strain_IS-98_STD(AF81864)_18mers_2MM.txt | WNV strain IS-98 STD(AF81864) |
| 412) | WNV_strain_NY99-eqhs(AF260968)_16mers_0MM.txt | WNV strain NY99-eqhs(AF260968) |
| 413) | WNV_strain_NY99-eqhs(AF260968)_16mers_1MM.txt | WNV strain NY99-eqhs(AF260968) |
| 414) | WNV_strain_NY99-eqhs(AF260968)_16mers_2MM.txt | WNV strain NY99-eqhs(AF260968) |
| 415) | WNV_strain_NY99-eqhs(AF260968)_17mers_0MM.txt | WNV strain NY99-eqhs(AF260968) |
| 416) | WNV_strain_NY99-eqhs(AF260968)_17mers_1MM.txt | WNV strain NY99-eqhs(AF260968) |
| 417) | WNV_strain_NY99-eqhs(AF260968)_17mers_2MM.txt | WNV strain NY99-eqhs(AF260968) |
| 418) | WNV_strain_NY99-eqhs(AF260968)_18mers_0MM.txt | WNV strain NY99-eqhs(AF260968) |
| 419) | WNV_strain_NY99-eqhs(AF260968)_18mers_1MM.txt | WNV strain NY99-eqhs(AF260968) |
| 420) | WNV_strain_NY99-eqhs(AF260968)_18mers_2MM.txt | WNV strain NY99-eqhs(AF260968) |
| 421) | WNV_strain_NY99-flamingo382-99(AF196835)_16mers_0MM.txt | WNV strain NY99- |
| | | flamingo382-99(AF196835) |
| 422) | WNV_strain_NY99-flamingo382-99(AF196835)_16mers_1MM.txt | WNV strain NY99- |
| | | flamingo382-99(AF196835) |
| 423) | WNV_strain_NY99-flamingo382-99(AF196835)_16mers_2MM.txt | WNV strain NY99- |
| | | flamingo382-99(AF196835) |
| 424) | WNV_strain_NY99-flamingo382-99(AF196835)_17mers_0MM.txt | WNV strain NY99- |
| | | flamingo382-99(AF196835) |
| 425) | WNV_strain_NY99-flamingo382-99(AF196835)_17mers_1MM.txt | WNV strain NY99- |
| | | flamingo382-99(AF196835) |
| 426) | WNV_strain_NY99-flamingo382-99(AF196835)_17mers_2MM.txt | WNV strain NY99- |
| | | flamingo382-99(AF196835) |
| 427) | WNV_strain_NY99-flamingo382-99(AF196835)_18mers_0MM.txt | WNV strain NY99- |
| | | flamingo382-99(AF196835) |
| 428) | WNV_strain_NY99-flamingo382-99(AF196835)_18mers_1MM.txt | WNV strain NY99- |
| | | flamingo382-99(AF196835) |
| 429) | WNV_strain_NY99-flamingo382-99(AF196835)_18mers_2MM.txt | WNV strain NY99- |
| | | flamingo382-99(AF196835) |
| 430) | WNV_strain_PaAn001_polyprotein(pol)_gene(AY268132)_16mers_0MM.txt | WNV strain |
| | | PaAn001 polyprotein(pol) gene(AY268132) |
| 431) | WNV_strain_PaAn001_polyprotein(pol)_gene(AY268132)_16mers_1MM.txt | WNV strain |
| | | PaAn001 polyprotein(pol) gene(AY268132) |
| 432) | WNV_strain_PaAn001_polyprotein(pol)_gene(AY268132)_16mers_2MM.txt | WNV strain |
| | | PaAn001 polyprotein(pol) gene(AY268132) |
| 433) | WNV_strain_PaAn001_polyprotein(pol)_gene(AY268132)_17mers_0MM.txt | WNV strain |
| | | PaAn001 polyprotein(pol) gene(AY268132) |
| 434) | WNV_strain_PaAn001_polyprotein(pol)_gene(AY268132)_17mers_1MM.txt | WNV strain |
| | | PaAn001 polyprotein(pol) gene(AY268132) |
| 435) | WNV_strain_PaAn001_polyprotein(pol)_gene(AY268132)_17mers_2MM.txt | WNV strain |
| | | PaAn001 polyprotein(pol) gene(AY268132) |
| 436) | WNV_strain_PaAn001_polyprotein(pol)_gene(AY268132)_18mers_0MM.txt | WNV strain |
| | | PaAn001 polyprotein(pol) gene(AY268132) |
| 437) | WNV_strain_PaAn001_polyprotein(pol)_gene(AY268132)_18mers_1MM.txt | WNV strain |
| | | PaAn001 polyprotein(pol) gene(AY268132) |
| 438) | WNV_strain_PaAn001_polyprotein(pol)_gene(AY268132)_18mers_2MM.txt | WNV strain |
| | | PaAn001 polyprotein(pol) gene(AY268132) |
| 439) | WNV_strain_RO97-50(AF260969)_16mers_0MM.txt | WNV strain RO97-50(AF260969) |
| 440) | WNV_strain_RO97-50(AF260969)_16mers_1MM.txt | WNV strain RO97-50(AF260969) |
| 441) | WNV_strain_RO97-50(AF260969)_16mers_2MM.txt | WNV strain RO97-50(AF260969) |
| 442) | WNV_strain_RO97-50(AF260969)_17mers_0MM.txt | WNV strain RO97-50(AF260969) |
| 443) | WNV_strain_RO97-50(AF260969)_17mers_1MM.txt | WNV strain RO97-50(AF260969) |
| 444) | WNV_strain_RO97-50(AF260969)_17mers_2MM.txt | WNV strain RO97-50(AF260969) |
| 445) | WNV_strain_RO97-50(AF260969)_18mers_0MM.txt | WNV strain RO97-50(AF260969) |
| 446) | WNV_strain_RO97-50(AF260969)_18mers_1MM.txt | WNV strain RO97-50(AF260969) |
| 447) | WNV_strain_RO97-50(AF260969)_18mers_2MM.txt | WNV strain RO97-50(AF260969) |
| 448) | WNV_strain_TVP_8533(AY289214)_16mers_0MM.txt | WNV strain TVP 8533(AY289214) |
| 449) | WNV_strain_TVP_8533(AY289214)_16mers_1MM.txt | WNV strain TVP 8533(AY289214) |
| 450) | WNV_strain_TVP_8533(AY289214)_16mers_2MM.txt | WNV strain TVP 8533(AY289214) |
| 451) | WNV_strain_TVP_8533(AY289214)_17mers_0MM.txt | WNV strain TVP 8533(AY289214) |
| 452) | WNV_strain_TVP_8533(AY289214)_17mers_1MM.txt | WNV strain TVP 8533(AY289214) |
| 453) | WNV_strain_TVP_8533(AY289214)_17mers_2MM.txt | WNV strain TVP 8533(AY289214) |
| 454) | WNV_strain_TVP_8533(AY289214)_18mers_0MM.txt | WNV strain TVP 8533(AY289214) |
| 455) | WNV_strain_TVP_8533(AY289214)_18mers_1MM.txt | WNV strain TVP 8533(AY289214) |
| 456) | WNV_strain_TVP_8533(AY289214)_18mers_2MM.txt | WNV strain TVP 8533(AY289214) |
| 457) | WNV_VLG-4_polyprotein_precursor(AF317203)_16mers_0MM.txt | WNV VLG-4 polyprotein |
| | | precursor(AF317203) |
| 458) | WNV_VLG-4_polyprotein_precursor(AF317203)_16mers_1MM.txt | WNV VLG-4 polyprotein |
| | | precursor(AF317203) |
| 459) | WNV_VLG-4_polyprotein_precursor(AF317203)_16mers_2MM.txt | WNV VLG-4 polyprotein |
| | | precursor(AF317203) |
| 460) | WNV_VLG-4_polyprotein_precursor(AF317203)_17mers_0MM.txt | WNV VLG-4 polyprotein |
| | | precursor(AF317203) |
| 461) | WNV_VLG-4_polyprotein_precursor(AF317203)_17mers_1MM.txt | WNV VLG-4 polyprotein |
| | | precursor(AF317203) |
| 462) | WNV_VLG-4_polyprotein_precursor(AF317203)_17mers_2MM.txt | WNV VLG-4 polyprotein |
| | | precursor(AF317203) |
| 463) | WNV_VLG-4_polyprotein_precursor(AF317203)_18mers_0MM.txt | WNV VLG-4 polyprotein |
| | | precursor(AF317203) |
| 464) | WNV_VLG-4_polyprotein_precursor(AF317203)_18mers_1MM.txt | WNV VLG-4 polyprotein |
| | | precursor(AF317203) |
| 465) | WNV_VLG-4_polyprotein_precursor(AF317203)_18mers_2MM.txt | WNV VLG-4 polyprotein |
| | | precursor(AF317203) |

Table A gives preferred values for some of the parameters of the invention.

TABLE A

| Parameter | Preferred | More Preferred | Most Pref |
|---|---|---|---|
| Host | Organism | Animal | Human |
| Input Sample | Body Fluids (blood, urine, | Body fluids, | Body fluids, |
| | saliva, sputum, sperm, | agricultural | PCR |
| | biopsy sample, forensic | products, | products |
| | samples, tumor cell, | microbial colonies, | |
| | vascular placques, transplant | PCR products | |
| | tiussues, skin, urinefeces); | | |
| | Agricultural Products | | |
| | (grains, seeds, plants, meat, | | |
| | livestock, vegetables, rumen | | |
| | contents, etc.); soil, air | | |
| | particulates; PCR products; | | |
| | purified nucleic acids, | | |
| | amplified nucleic acids, | | |
| | natural waters, contaminated | | |
| | liquids; surface scrapings or | | |
| | swabbings; Animal RNA, | | |
| | cell cultures, pharmaceutical | | |
| | production cultures, CHO | | |
| | cell cultures, bacterial | | |
| | cultures, virus-infected | | |
| | cultures, microbial colonies | | |
| Target organisms | 0-100 | 2-20 | 1-2 |
| per sample | | | |
| Target sequence | SSU RNAs, LSU-rRNAs, | 16S rRNA, Virus | 16S rRNA |
| type | 5S rRNA, spacer region | RNA, Virus DNA | |
| | DNA from rRNA gene | rRNA gene cluster | |
| | clusters, 5.8S rRNA, 4.5S | spacer region DNA | |
| | rRNA, 10S RNA, RNAseP | | |
| | RNA, guide RNA, | | |
| | telomerase RNA, snRNAs- | | |
| | e.g. U1 RNA etc, scRNAs, | | |
| | Mitochondrial DNA, | | |
| | Virus DNA, virus RNA | | |
| | PCR product, human DNA, | | |
| | human cDNA, artificial | | |
| | RNA, siRNA | | |
| Organism | Bacterium, virus, plant, | Bacterium, | Virus |
| | animal, fungus, yeast, | Archaea, | |
| | mold, Archae; Eukyarotes; | eukaryotic | |
| | Spores; Fish; Human; | microorganisms | |
| | Gram-Negative bacterium, | virus | |
| | Y. pestis, HIV1, B. | | |
| | anthracis, Smallpox virus | | |
| Nucleic Acid | Chromosomal DNA; | rRNA, Viral RNA, | rRNA |
| | rRNA; rDNA; cDNA; mt | Viral DNA | |
| | DNA, cpDNA, aRNA, | | |
| | plasmid DNA, | | |
| | oligonucleotides; PCR | | |
| | product; Viral RNA; Viral | | |
| | DNA; restriction | | |
| | fragment; YAC, BAC, | | |
| | cosmid | | |
| Probe length | 5 to 2500 | 7 to 20 | 10 to 20 |
| Number of | 1-100,000,000 | 20-100,000 | 50-10,000 |
| probes | | | |
| Classification | Kingdom; Phylum; Class; | Genus; Species, | Strain, |
| Level | Order; Family; Genus; | Strain | Species |
| | Species; Subgroups; | | |
| | Strain, Tribe, Serotype; | | |
| | Gram stain | | |
| Utility | Clinical Diagnosis; | Clinical Diagnosis; | Clinical |
| | Pathogen discovery; | Biodefense; | Diagnosis |
| | Biodefense; Research; | Adulterant | |
| | Adulterant Detection; | Detection | |
| | Counterfeit Detection; | | |
| | Food Safety; Taxonomic | | |
| | Classification; | | |
| | Microbial ecology; | | |
| | Environmental | | |
| | Monitoring; Agronomy; | | |
| | Law Enforcement | | |
| Sample | acid, base, detergent, | Polymerase, | Polymerase, |
| preparation | phenol, ethanol, | restriction | phenol |
| Agent | isopropanol, chaotrope, | endonuclease, | |
| | enzyme, protease, | Phenol | |
| | nuclease, polymerase, | | |
| | adsorbent, ligase, primer, | | |
| | nucleotide, restriction | | |
| | endonuclease, detergent | | |
| Sample | Filter, Centrifuge, Extract, | Filter, centrifuge, | Filter, |
| Preparation | Adsorb, protease, | culture | culture |
| Pretreatment | nuclease, partition, wash, | | |
| | leach, lyse, electrophoresis, | | |
| | precipitate, germinate, | | |
| | Culture | | |
| Hybridization | Aqueous buffer, solution | Aqueous buffer, | Solution |
| Media | containing formamide, | solution | containing |
| | zwitterion solution, heated | containing | formamide, |
| | solution, alcohol solution | formamide, heated | heated |
| | | solution | solution |
| Cultivation | LB, M9, blood agar, | LB, blood agar, | Blood agar |
| Media | DMEM, calf serum | Culture medium | |
| | medium, McConkey's | containing host | |
| | medium, Culture medium | cells | |
| | containing host cells | | |
| Separation media | Ion exchanger, filter, | Ion exchanger, | Ion |
| for sample | ultrafilter, depth filter, | multiwell filter, | exchanger, |
| preparation | multiwell filter, centrifuge | immobilized-metal | silica, |
| | tube, multiwell plate, | affinity adsorbent, | magnetic |
| | immobilized-metal affinity | multiwell plate, | beads |
| | adsorbent, hydroxyapatite, | hydroxyapatite, | |
| | silica, zirconia, magnetic | silica, magnetic | |
| | beads | beads | |
| Detection Means: | Mass Spec.; Fluorescence; | | Fluorescence |
| (Probe | Chemiluminesence; | | |
| Hybridization): | Enzyme Reaction; | | |
| | Radiochemical; Self- | | |
| | quenching Probe | | |
| | hybridization; Surface | | |
| | Plasmon Resonance; Total | | |
| | Internal Reflection | | |
| | Fluorescence; Liquid | | |
| | Crystals; Magnetic; | | |
| | Infrared; Array Detection | | |
| | Peptide Nucleic Acid | | |
| | hybridization; Branched | | |
| | DNA hybridization; Redox | | |
| | Chemistry; LNA | | |
| | hybridization, PNA | | |
| | hybridization, array, bead | | |
| | array | | |
| Detection Means: | Mass Spectrometry; | | |
| (Nonhybridization | Electrophoresis; Affinity | | |
| Methods) | electrophoresis; | | |
| | Chromatography, HPLC; | | |
| | DHPLC; Neutron | | |
| | Activation Analysis | | |

### Utilities

A feature of this invention is our newly developed ability to calculate the occurrence of all sub-sequences within DNA genomes. The present invention builds on this ability with the recognition that if we can calculate the occurrence of all sub-sequences in the human genome, then we can design diagnostics targeting only sequences known to be absent from that genome, thereby avoiding the enormous background of human DNA which plagues many clinical diagnostic activities.

There are many other applications that look like this one, and these fall into two general categories. The first application, which we will call “rifle shot”, application consists of targeting a known organism or sequence of interest in the presence of (usually an excessive quantity of) some background DNA. For example, we could use the known sequences of the hepatitis C and human genomes to design primers which PCR-amplify hepatitis C nucleic acids while not amplifying human DNA. It bears emphasis that this design is relatively easy once the enormous computational achievement of identifying all the primers which can amplify human DNA has been accomplished. There are many, many extensions and modifications of this class of application. First, it should be noted that human genome is not a single entity but includes a large range of variation known as single nucleotide polymorphisms (“SNPs”). These variations can be taken into account in the computations, and the computations will be updated as more are reported. For the moment, the computations will normally produce candidate probes and primers that will then be experimentally tested because of variations in hybridization efficiency and the occurrence of unknown genetic variations in humans. In addition to designing PCR primers or DNA probes for known pathogens, it is also possible to identify organisms of agricultural interest in animal-derived samples, pathogens in meat (e.g. avoiding cow sequences, once all or most of the cow genome is known), tracking organisms of environmental interest, distinguishing a small quantity of human Y-chromosome—derived material from excess female genetic material in forensic cases, diagnosing plant diseases, monitoring malaria parasite DNA in mosquitoes, testing for contamination in transplant tissues and pharmaceutical manufacturing samples, etc.

The other class of applications is those in which the background sequences are known, but the precise sequence to be looked for is not yet completely identified. These we term “butterfly net” applications. One recent example would be the outbreak of SARS. In principle we could design rather large sets of primers capable of randomly amplifying non-human DNA present in human blood samples. Application of this PCR primer set to blood samples taken from SARS patients could result in the amplification of previously unknown DNA sequences that would be the SARS genome, rapidly advancing the identification and the diagnosis of this disease. Biodefense and bioterrorism applications of this approach are also evident. Another butterfly net application of historical importance would be the identification of Helicobacter pylori as a causative agent of human gastric ulcers. In this case, we could have amplified Helicobacter genomic DNA from biopsy specimens (or possibly gastric, intestinal, or colon content samples). Currently interesting applications include identification of emerging diseases and biodefense agents, and causative agents of upper respiratory infections, vascular disease, cancers, etc.

It bears emphasis that specific primers and probes to be developed here can be used in a variety of manifestations. In general, these divide into amplification schemes and hybridization assays.

Amplification schemes will include especially PCR but also RT-PCR, NASBA, LCR, transcription, and a variety of other known amplification schemes. It also bears notice that RNA and DNA may be interconverted as an early step in the assay, and that isolation of nucleic acids may in many cases be quite useful to facilitate the hybridization or reactions used, by concentration and removal of inhibitors.

The second general class of applications is in hybridization assays in which sequences are detected directly. Hybridization may be preceded by several purification and/or amplification. Hybridization may be performed in a spotted or grown array of probes, and these probes may be oligonucleotides, PCR products or spotted natural nucleic acids. Arrays may commonly be in the usual planar or 3-D gel pad format, but they may also consist of arrays of beads scored either by imaging or cytometric technique. Both probes and primers may consist of non-natural nucleic acids or nucleic acid analogs modifications may include alterations of backbone, fluorescence reporters, beacon probes, aminopurine and other fluorescent reporter nucleotides, and perhaps most importantly variations such as LNAs and PNAs.

In most applications the computational techniques based on the Fofanov R-Q set calculations will yield a candidate set of primers of interest which will then be polished experimentally to avoid difficulties with unknown variations in background DNA or unexpected behavior in hybridization. Polishing of the candidate set will usually be experimental and will consist of hybridization or test amplification reactions. The calculations will produce an abundance of candidates, and it is possible and desirable to apply additional criteria, especially for predicted hybridization/melting temperature, mismatch location, etc. Constraining the range of predicted melting temperature of probes or primers facilitates their use in subsequent amplifications and assays, and this constraint is fairly easily applied by searching among the candidate probes or primers for those containing similar G+C content, or by more sophisticated melting temperature estimates which take into account neighbor-neighbor interactions (e.g., using nearest neighbor parameters published in Breslauer, Frank, Bloeker and Marky, Proc. Natl. Acad. Sci. USA, vol 83, pp 3746-3750) and mismatch (MM) location. In solution-based assays such as PCR, it will often be important to filter sequences by possible complemetarity to each other, to separation distance of target, or by chromosomal/episomal location of target sequence.

It is worth noting that there are a variety of modifications and extensions of the technology including miniaturization, parallelization, scoring by beacon fluorescence, scoring by radionuclide incorporation, scoring of hybridization by fluorescence, by antibody, by chemiluminescence, by electrical signals such as capacitance or impedance or conductivity. A huge variety of technologies are known in the literature for conducting amplification and/hybridization based assays and nearly all of these are applicable with the present invention.

Another class is the compositions of matter of the primers of interest, especially primers known not to hybridize to or amplify known human sequences including the publicly available human genome sequence and its SNP-derived variants (or primers selected not to hybridize to other background(s) which may be present). A particularly interesting group of primers are those of selected length (e.g., 6-, 9-, 14-, 15-, 16-, 17-, or 24-mers) selected for similar melting temperatures to make a set of primers and/or probes which could be used under similar conditions and in parallel or high throughput manner. These sequences have known utility and are well defined in terms of composition, we submit them on CD to allow for the large numbers which we'll identify.

### [6. Scope of Potential Use]

The database of subsequences of microbial and viral genomes (6-20+-mers) will have broad applications by a variety of communities. In addition to the area of microbial identification, other areas of application include evolution and phylogeny, genome stability, viral strain origins, environmental microbiology, infectious diseases, food safety, DNA-binding proteins, restriction and modification systems (and practical applications in molecular biology), siRNA, forensics, biodefense, and ribosome function.

The database of subsequences of microbial and viral genomes (6-25+-mers) that are absent in the human genome will provide convenient and accurate genomic signatures for a large and ever increasing number of microbial and viral genomes. One can quickly obtain these signatures and develop a set of primers or probes to target the microbial or viral species of interest. Furthermore, since the subset of subsequences that are present in the human genome are eliminated, one can develop probing methods not requiring removal of human tissue from the sample. Any research lab that is interested in identifying and investigating one or more microbial or viral species can benefit from this resource.

With our database of genome subsequences, one can develop a set of microarray probes which will provide a distinctive pattern in the presence of any known pathogenic microbial or viral species, even without isolation of pathogen DNA from infected human tissue. Similar applications in mRNA expression monitoring can be envisioned, where RNA processing gives rise to sequences not present in the genomic DNA, but which can be calculated using standard bioinformatics methods, or in many cases accessed from cDNA databases.

### EXAMPLES

### Database of Genomic Subsequences and Ultra-Specific Probes for Microbial Detection

### Example 1

We develop a set of novel algorithms that make it possible to analyze the occurrence frequency of all short subsequences (“n-mers”) of length 5 to 25+ nucleotides in any genome within a reasonable time (minutes). These algorithms are used to perform a comparative statistical analysis of the presence of all possible “n-mers” in genomes of more than 250 microbial, viral and multicellular organisms (including humans)^{26,27} The results show a remarkable similarity of presence/absence distributions for different n-mers in all genomes. It suggests that the presence/absence distribution of n-mers in all genomes considered (provided that the condition M<<4ⁿ holds, where M is the total genome sequence length) can be treated as nearly random. The massive computational analysis of the presence/absence of short subsequences in more than one genome simultaneously is performed for all published (before May, 2002) microbial and virus genomes^{28,29}, and is repeated for the 1600+ genomes which were available by May 2003. Results show that for organisms that are not close relatives, the presence/absence of different 7-20-mers in their genomes are not correlated. For close biological relatives, some correlation of the presence of n-mers appears, but is not as strong as expected. The low level of correlation among the n-mers present in different genomes leads to the possibility of using random (quasi-random) sets of n-mers (with appropriately chosen n) to discriminate genomes of different organisms (and possibly individual genomes of the same species including humans) with a low probability of error^{29,30}.

We work on the first generation of microbial/viral biosensor microarrays based on these special properties of genomic n-mers. The general long-term object is to develop laboratory technology to rapidly and conclusively identify any organism. As a part of this effort, considerable improvement of the n-mers computation algorithms is achieved. It is now possible to exclude in the array design step all subsequences which are present in the human genome and publicly available human SNP database. This improvement makes it possible to refocus the project on development of “human-blind” probes and primers, instead of just random primers. This new approach can readily be extended to develop assays which are insensitive to the background of a host such as a food animal or plant, host organism, or any environmental background for which genomic sequence information is available. It can be used to design and manufacture a DNA microarray, which will allow one to distinguish pathogen (microbial or viral) DNA from a sample that contains a host DNA (for instance, blood, and tissue samples from an animal or human). Indeed, in the event of microbial infection, it would be of value to have a diagnostic system that could readily identify any bacterium that is present in the host organism, regardless of prior expectations of what might be found. It would be essential to determine as closely as possible, the genetic identity of the organism (microbial or viral) that is causing the problem as this will clarify where the organism came from, what treatments are likely to be effective, etc. Since more than 1600 genomes have been collected at the UH Bioinformatics lab and more than 72,600 16S rRNA sequences are available from the RDP, it appears possible to find all “human blind” subsequences of size 8 to 25+.

Critical to development of this technology is the ability to identify and use sequences which do not hybridize to human DNA, including known SNPs (there will be some false positives due to unknown SNPs, but these will be few and the number will decrease with SNP database growth). The basic idea is to use a set of human-blind signature n-mer sequences for microbial/viral identification. Even if each such sequence is not exclusive, and could be present in many genomes, the combination of sequences present is exclusive for each genome. As a consequence, it is possible to use a random subset of human-blind n-mers (with appropriately chosen n) to design a microarray to diagnose to which organism a given DNA/RNA sample belongs. As shown in^{29,30}, the size of this microarray would be several thousand oligonucleotides, which is well within the range of current experimental methods.

An important advantage of this approach is that it can be used without a priori knowledge of the genomic sequence of the target organism. In other words, one can identify organisms for which the total genome sequences have not yet been determined. It is sufficient to obtain the purified DNA, hybridize it on the microarray chip and check which n-mers show up (they provide a signature of an organism on our chip). Taking into account the availability of standard samples of microbes and viruses, how easily each microarray can be produced, and the fact that one does not need to determine quantitative values of expression (only a yes/no answer is required)—a universal human-blind microbial/viral DNA identification chip can be readily produced.

The amount of computation necessary to find all the short subsequences present in any target genome, and absent in the human or another host genome, is substantial and cannot be readily performed whenever needed. Given that those interested in such data will typically be experimental laboratories rather than computational ones, the need to provide public access to the results is clear. Various pathogen genomes, the ability to find information regarding the location of each signature in each genome, the ability to find sets of unique signatures and PCR primers that are expected to allow ultra-specific microbe genetic material amplification will add useful information to scientific community. This technology may be useful in developing new nucleic acid-based diagnostics, forensic assays, veterinary and agricultural assays. It will also have considerable utility in rapidly identifying unknown pathogens or bio-warfare agents.

Statistical analysis of the appearance of short subsequences of length n, called motifs or n-mers, in different DNA sequences from individual genes to full genomes, is of interest in terms of evolutionary biology, analysis of regulatory sites on DNA promoter sequences, etc. In addition, knowledge of the distribution of presence of n-mers is necessary for PCR primer^{32,33} and microarray probe design³⁴. Several attempts³⁵⁻⁴¹ have been made to employ the frequency distributions of n-mers to identify species with relatively short genome sizes (microbes). In such an approach, the shape of the frequency distribution for particular short subsequences (2-4-mers^{35-39,42} and 8-9-mers^{40,41}) has been proposed as a criterion to determine which microbial genome is present, based on a given random piece of a genome or a whole genome. It has been reported that when genome size M is greater than 4ⁿ, the appearances of n-mers in various genomes are not random^{35,36,38,39,43}. The basic motivation of our recent analysis was to explore the statistical properties of the presence of longer n-mers if genome size M is much less than 4^{n 26,27,29-31}.

### Example 2

We examine the number of all distinct 5-20-mers present in more than 1600 viral and microbial genomes available^{26,27,29-31.} We also analyze the genomes of several multicellular organisms, including human⁴⁴. Tables 1 and 2 contain typical results for some of the analyzed genomes (microbial and viral), for n=8 and 12. The numbers of 7-20 mers present in the human genome can be found in Table 3.

TABLE 1
Frequency of presence of 8-mers and self-similarity value (see definition in the
text) for several viral genomes.

| | | | | Frequency | | |
|---|---|---|---|---|---|---|
| | | Total | Number | of | | |
| | | Sequence | of presence | presence | Random | Self- |
| Accession | Genome | length | 8-mers | 8-mers | boundary | similarity |
| NC_001436 | Human T-cell lymphotropic | 17,014 | 13,739 | 20.96% | 22.86% | 8.31% |
| | virus type 1 | | | | | |
| NC_001707 | Hepatitis B virus | 6,430 | 5,963 | 9.10% | 9.35% | 2.64% |
| NC_001503 | Mouse mammary tumor | 17,610 | 14,307 | 21.83% | 23.56% | 7.35% |
| | virus | | | | | |
| NC_001434 | Hepatitis E virus | 7,176 | 6,517 | 9.94% | 10.37% | 4.12% |
| NC_001489 | Hepatitis A virus | 7,478 | 6,543 | 9.98% | 10.78% | 7.42% |
| NC_003461 | Human parainfluenza virus 1 | 15,600 | 12,242 | 18.68% | 21.18% | 11.82% |

TABLE 2
Frequency of presence of 12-mers and self-similarity value for several
microbial genomes.

| | | | | Frequency | | |
|---|---|---|---|---|---|---|
| | | Total | Number | of | | |
| | | Sequence | of present | present | Random | Self- |
| Accession | Genome | length | 12-mers | 12-mers | boundary | similarity |
| NC_000964 | Bacillus subtilis | 8,429,628 | 5,346,103 | 31.87% | 39.50% | 19.32% |
| NC_000913 | Escherichia coli K12 | 9,278,442 | 5,695,881 | 33.95% | 42.48% | 20.08% |
| NC_003197 | Salmonella typhimurium | 9,714,864 | 5,821,910 | 34.70% | 43.96% | 21.06% |
| | LT2 | | | | | |
| NC_002758 | Staphylococcus aureus | 5,756,080 | 3,398,622 | 20.26% | 29.04% | 30.25% |
| | Mu50 | | | | | |
| NC_003098 | Streptococcus pneumoniae | 4,077,230 | 2,992,091 | 17.83% | 21.57% | 17.34% |
| | R6 | | | | | |
| NC_002689 | Thermoplasma volcanium | 3,169,608 | 2,590,718 | 15.44% | 17.22% | 10.30% |
| NC_002162 | Ureaplasma urealyticum | 1,503,438 | 948,274 | 5.65% | 8.57% | 34.06% |

When n increases, the total number of possible n-mers (4ⁿ) strongly exceeds the total sequence length M and most of the possible n-mers do not appear at all because the maximum number of n-mers contained in this sequence is M−n+1≈M. Moreover, for a reasonably high ratio, 4ⁿ/M, most of the n-mers which appear tend to appear only once, in accordance with the fact that the number of n-mers present becomes very close to M. This is why we have chosen to use the statistics for “presence/absence” (frequency of presence) in our analysis instead of the usual “frequency of appearance”, which is reasonable for short n-mers (total sequence length M>4ⁿ).

TABLE 3
Presence/absence statistics for human genome of size 2,874,736,094 base pairs.
Calculation was performed using both (original and complementary) DNA strand
sequences.

| n- | Number of | | | | | Number of n-mers | |
|---|---|---|---|---|---|---|---|
| mer | different n-mers | Number of absent n- | | Number of n-mers | | present more than | |
| size | 4ⁿ | mers | | present only once | | once | |
| 7 | 16,384 | 0 | 0.00% | 0 | 0.00% | 16,384 | 100.00% |
| 8 | 65,536 | 0 | 0.00% | 0 | 0.00% | 65,536 | 100.00% |
| 9 | 262,144 | 0 | 0.00% | 0 | 0.00% | 262,144 | 100.00% |
| 10 | 1,048,576 | 0 | 0.00% | 0 | 0.00% | 1,048,576 | 100.00% |
| 11 | 4,194,304 | 42 | 0.00% | 324 | 0.01% | 4,193,938 | 99.99% |
| 12 | 16,777,216 | 42,501 | 0.25% | 91,146 | 0.54% | 16,643,569 | 99.20% |
| 13 | 67,108,864 | 2,382,096 | 3.55% | 2,642,582 | 3.94% | 62,084,186 | 92.51% |
| 14 | 268,435,456 | 41,634,971 | 15.51% | 30,411,367 | 11.33% | 196,389,118 | 73.16% |
| 15 | 1,073,741,824 | 410,828,287 | 38.26% | 166,998,278 | 15.55% | 495,915,259 | 46.19% |
| 16 | 4,294,967,296 | 2,717,880,983 | 63.28% | 671,192,253 | 15.63% | 905,894,060 | 21.09% |
| 17 | 17,179,869,184 | 14,452,040,667 | 84.12% | 1,790,043,813 | 10.42% | 937,784,704 | 5.46% |
| 18 | 68,719,476,736 | 65,147,397,575 | 94.80% | 2,881,849,256 | 4.19% | 690,229,905 | 1.00% |
| 19 | 274,877,906,944 | 270,850,664,602 | 98.53% | 3,538,156,028 | 1.29% | 489,086,314 | 0.18% |
| 20 | 1,099,511,627,776 | 1,095,257,688,530 | 99.61% | 3,866,031,543 | 0.35% | 387,907,703 | 0.04% |

### Example 3

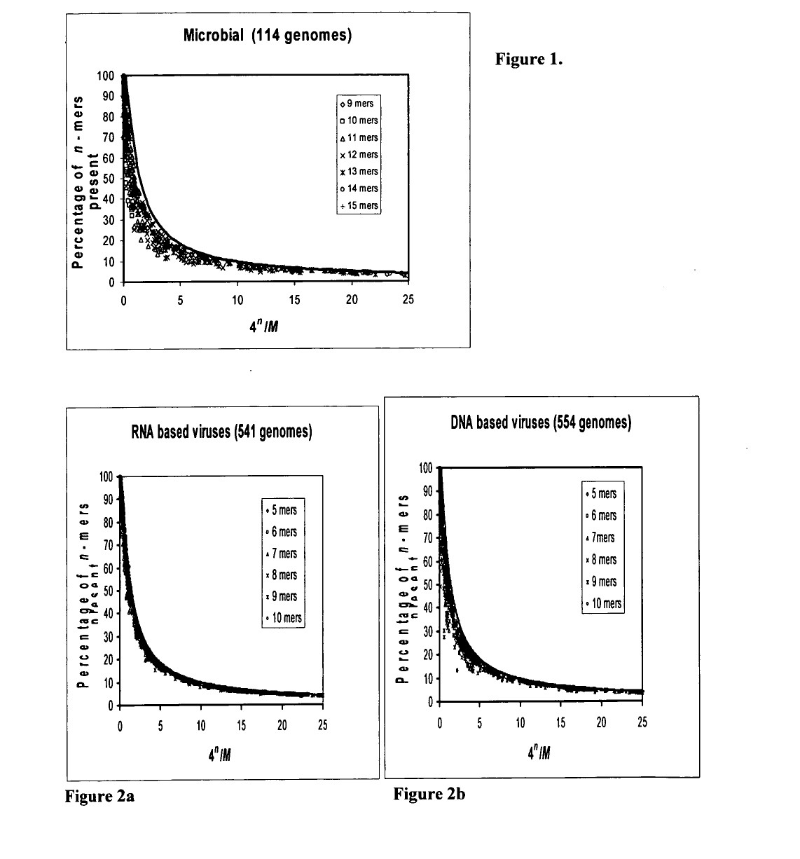

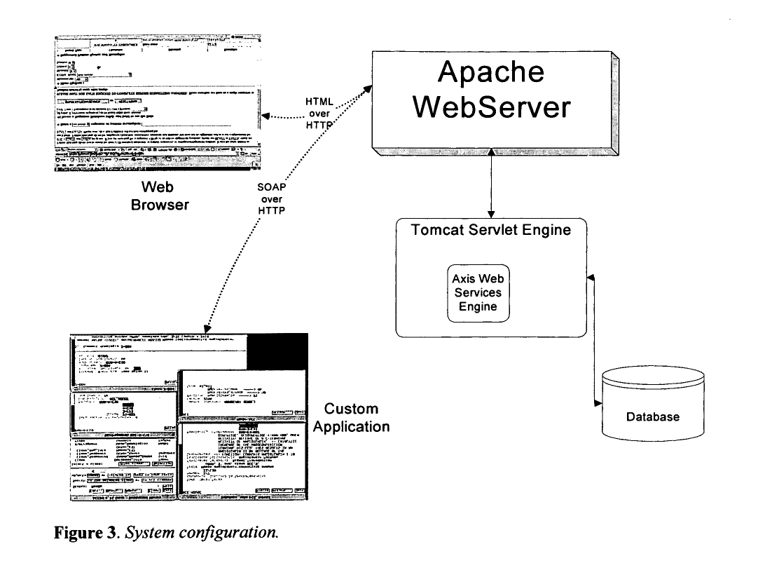

Based on data from Example 2, FIGS. 1 and 2 are plots of the frequency of presence, f; of n-mers in genomes (the number of different n-mers present in a given genome over the total number of n-mers, 4ⁿ) against the ratio 4ⁿ/M. FIG. 1 corresponds to the microbial genomes, and FIG. 2 to RNA- and DNA-containing viruses. All of the different genomes form a well-defined pattern, when plotted against the ratio 4ⁿ/M and not against the size of the genome or the length of the n-mers separately. This similarity was first observed in analyzing 250+ microbial and viral genomes²⁹⁻³¹ and led us to the hypothesis that the frequency f of presence/absence of relatively long n-mers (M<4ⁿ) can be treated as a result of a random process, or at least may contain a strong random component. In order to test this suggestion we extended our calculations to 1600+ genomes available, and found remarkable agreement with our hypothesis.

In fact, the frequencies of presence of n-mers, f, in various genomes nearly belong to the same universal curve representing the “random boundary” (always being below it). Assuming equal probabilities of appearance of every nucleotide, one can analytically find (in full agreement with the Monte-Carlo simulations which we also performed as an independent test),

$${f}_{0} = 1 - exp ( - \frac{1}{x} ) , x = \frac{{4}^{″}}{M} , \qquad ( 1 )$$

where f₀ is M, x is the ratio of the total number of possible n-mers to the number of n-mers in the sequence in consideration, M−n+1≈M. This “random boundary” is shown in FIGS. 1 and 2 as a solid line.

The relative deviation

$$D = 1 - \frac{f}{{f}_{0}}$$

of real results from the random boundary can be used as a definition of “non-randomness”, or “self-similarity” of a given genome.

Corresponding data for several genomes are given in Tables 1-2 and in supplementary data to Refs.^{30,31}. We observe that, in general, shorter genomes are more random (based on this definition) than longer ones.

As shown in FIG. 2, the average value of the relative deviation, D, for 500+ RNA viruses calculated for 7-8 mers is less than 7%, for 500+ DNA viruses for 8-10-mers it is less than 12%, and for 114 microbial genomes for 11-12-mers it is less than 30%. For the five large genomes considered, the most “non-random” behavior (the largest self-similarity) is demonstrated by the human genome: 50.2% for 16-mers and the rice genome: 45.9% for 15-mers. The next is C. elegans: 40.5% for 14-mers. The most “random” is Drosophila melanogaster: 24.8% for 14-mers.

### Example 4

The analysis of the presence/absence of short subsequences in several genomes simultaneously was performed for 250+ microbial, viral, and multicellular organism genomes^{26,27,29-31}, and further repeated our calculations for the 1600+ genomes. We examined all possible short nucleotide subsequences of various lengths (5<n<20). To the best of our knowledge, no such studies appear in the literature for n>11. This type of calculation is challenging for larger n because of the exponential growth of time/memory required by brute force algorithms. To be able to perform calculations for n>11, new algorithms and specialized data structures have been developed and implemented^{26,27}. Our goal was to find out how independent/correlated the appearances of n-mers are in different genomes. To answer this question we used the multiplication property for the joint probability of the intersection of events, according to which two events A, and B, can be treated as independent if p(A∩B)=p(A)p(B).

Consider a simple example based on 3 different genomes: (1) Salmonella typhi (NC_003198), (2) Mycobacterium tuberculosis H37Rv (NC_000962), and (3) Bacillus subtilis (NC_000964). A complete set of n-mers would contain 4ⁿ n-mers, which, for n=12, is 4¹²=16,777,216. We use both strands of the complete genome sequences for our calculations, and N(n,G) stands for the number of different n-mers in genome G. In Table 4 we present the number of different 12-mers that occur in each of these three genomes.

### Example 5

Based on the data of Examples 1-4, to estimate the probability of finding randomly chosen 12-mers in each genome, we calculate the frequency of presence of 12-mers in each genome (p=N(12,G)/4ⁿ). These values are also presented in Table 4. A moderate percentage can be observed when compared with the maximum of possible sequences, 4ⁿ.

TABLE 4
The frequency of presence of 12-mers
within the 3 microbial genomes.

| | | | Number of | |
|---|---|---|---|---|
| | | | different 12- | |
| | | Total | mers present | |
| | Genome | sequence | in genome: | p = |
| Genome (G) | length | length | N(12, G) | N(12, G)/4ⁿ |
| (1) Salmonella typhi | 4,809,037 | 9,618,074 | 5,813,330 | 34.65% |
| (2) Mycobacterium | 4,411,529 | 8,823,058 | 4,361,508 | 26.00% |
| tuberculosis H37Rv | | | | |
| (3) Bacillus subtilis | 4,214,814 | 8,429,628 | 5,346,103 | 31.87% |

The number N(n, G₁, G₂) of n-mers (n=12) that appear in each pair of genomes (G₁, G₂) was also computed (Table 5). Based on this we were able to compare the probabilities of finding randomly picked 12-mers in each pair of genomes with the probabilities calculated using the multiplication rule. As is shown in Table 5, the actual and calculated (expected) probabilities do not differ greatly from each other. This allows us to treat the presence/absence of randomly picked 12-mers in these 3 genomes as independent events.

### Example 6

TABLE 5
Actual and predicted simultaneous presence of 12-mers within
the 3 microbial genomes: (1) Salmonella typhi, (2)
Mycobacterium tuberculosis H37Rv, and (3) Bacillus subtilis

| | | | Probability |
|---|---|---|---|
| | Number | N(n, G₁, | assuming |
| Case | 12-mers | G₂)/4ⁿ | independence |
| Present in genomes | 1,943,814 | 11.6% | 9.0% |
| (1) and (2) | | | |
| Present in genomes | 2,335,710 | 13.9% | 11.0% |
| (1) and (3) | | | |
| Present in genomes | 1,334,288 | 8.0% | 8.3% |
| (2) and (3) | | | |

Based on Example 5, we calculate the actual and expected probabilities for each pair of genomes in the three groups (microbes, DNA and RNA based viruses)²⁹⁻³¹ for values of n from 7 to 20. For all organisms which are not extremely closely related to each other, we find that in a certain range of n the presence/absence of n-mers can be treated as almost independent events. Moreover, this property is valid for almost any subset of n-mers such as, for instance, n-mers with a given C+G content, a subset of n-mers chosen according to Tm (melting temperature), etc.

Our calculations show that very similar results can be achieved using a “host blind” subset of n-mers chosen not to be present in the human genome. The only problem in this case occurs not because of the amount of host DNA, but because of the large difference between the lengths (sequence complexities) of human and microbial DNAs. For example, using 11-mers to detect microbes against a human DNA background is nearly impossible, because practically all 11-mers are present in the human genome. Nevertheless, it can be done be using 14 or 15-mers, (only 84.5% of 14-mers and 61.7% of 15-mers are present in the human genome; see Table 3 for more details). In this case one needs to increase the number of probes on the microarray to 10,000-15,000, which is technically possible. Such an approach also can be used to detect unknown viruses, but it requires the use of a still larger (but technically feasible) array.

As described above, the steady accumulation of genomic sequence information can transform the utility of DNA probes for microbial identification. An array (or set of PCR primers) designed to be blind to the background DNA allows extremely efficient and powerful microbial identification. The important advantage of such an approach is that it can be used without a priori knowledge of the target organism's genomic sequence. This implies that there is no need to perform the expensive and time-consuming process of sequencing before array design. It is enough to obtain the purified DNA, hybridize it to a sufficiently random microarray chip and check which n-mers are present. Considering the rapid growth in genomic databases, the increasing availability of custom DNA probe arrays, and the fact that we do not need to determine quantitative values of expression (we need just a yes/no answer) we believe it is possible to produce an essentially universal microbial/viral DNA chip. The oligonucleotide calculations and database proposed here would radically advance microbial detection and identification, as well as serving a wide variety of applications in the analysis of genome structure, evolution, and the regulation of gene expression.

### Example 7

Subsequences of each of the known sequences of Ebola virus are determined, and sequences common to nearly all strains of the virus and not found in the human sequence or known SNPs are identified. Sequences absent from the human genome and from all but one known Ebola sequence are also identified. All-strain- and single-strain-characteristic complementary probe sequences are immobilized on a microscope slide, and hybridized with labeled human clinical samples. Positive hybridization to both types of probes identifies the presence of the Ebola virus, and the presence of the particular strain of interest.

### Example 8

Subsequences of each of the known sequences of Ebola virus are determined, and sequences common to nearly all strains of the virus and not found in the human sequence or known SNPs are identified. Sequences absent from the human genome and from all but one known Ebola sequence are also identified. All-strain- and single-strain-characteristic probe sequences selected for similar predicted melting temperature are immobilized on a microscope slide, and hybridized with labeled RNA amplified from human clinical samples. Negative hybridization to both types of probes confirms the absence of the Ebola virus.

### Example 9

Subsequences of each of the known sequences of Ebola virus are determined, and sequences common to nearly all strains of the virus and not found in the human sequence or known SNPs are identified. 19-mer sequences absent from the human genome and from all but one known Ebola sequence are also identified. Among these sequences, the subset not convertible into a perfect match for any human sequence or SNP-modified sequence by any combination of 1, 2 or 3 mismatches are selected and probes to them are immobilized on a microscope slide, and hybridized with labeled human clinical samples. Positive hybridization to both types of probes identifies the presence of the Ebola virus, and the presence of the particular strain of interest.

### Example 10

Sequences absent from the human genome and all known human SNPs, and not convertible into a perfect match for any human sequence or SNP-modified sequence by any combination of 1, 2 or 3 mismatches are identified, and a subset of these sequences with predicted hybridization melting temperature in the range 62-65 degrees C. and limited propensity to hybridize to each other is synthesized and blended in to a PCR mixture. Nucleic acids isolated from liver biopsies from patients showing hepatitis of unknown origin are added to the mixture and aliquots subjected to PCR under a range of conditions. Sequences from a previously unknown virus distantly related to the Hepatitis C virus are amplified.

### Example 11

Sequences absent from the human genome and all known human SNPs, and not convertible into a perfect match for any human sequence, SNP-modified sequence or the genes encoding production of a therapeutic antibody by any combination of 1, 2 or 3 mismatches are identified, and primers to a subset of these sequences with predicted hybridization melting temperature in the range 62-65 degrees C. is synthesized and blended in to a PCR mixture. Nucleic acids isolated from human cell line cultures used in production of the therapeutic antibody are added to the mixture and aliquots subjected to PCR under a range of conditions. The absence of amplification (and successful amplification of spiked positive control mixtures) is used as a quality control measure before release of the production batch.

### Example 12

Sequences predicted to be present in human mRNA based on cDNA databases and genome annotation, but absent from the human genomic DNA sequence and all known human SNPs and not convertible into a perfect match for any human genomic DNA sequence or SNP-modified sequence by any combination of 1 or 2 mismatches are identified. Probes to a subset of these sequences with predicted DNA probe/mRNA hybridization melting temperature in the range 63-70 degrees C. are synthesized as DNA probes and spotted onto a microscope slide, and hybridized with labeled mRNA amplified from human leukemia samples. The pattern of gene expression inferred from the hybridization results is used to choose the course of treatment of the leukemia.

### Example 13

19-mer sequences in the genomic DNA of a pathogenic organism but absent from the human genome and all known human SNPs, and not convertible into a perfect match for any human sequence or SNP-modified sequence by any combination of 1 or 2 mismatches are identified. The process is repeated for each of 30 known human pathogens, and for each pathogen probes to a subset of these sequences with predicted hybridization melting temperature in a selected range are synthesized. An array of all these sequences is used in the analysis of human cerebrospinal fluid for diagnosis of meningitis.

### Example 14

Subsequences of each of the known sequences of Ebola virus are determined, and sequences common to nearly all strains of the virus and not found in the human sequence or known SNPs are identified. 19-mer sequences absent from the human genome and from all but one known Ebola sequence are also identified. Among these sequences, the subset not convertible into a perfect match for any human sequence or SNP-modified sequence by any combination of 1, 2 or 3 mismatches are selected and corresponding probes are immobilized on a microscope slide, and hybridized with labeled human clinical samples. Although not all Ebola-specific probes give positive signals, statistical analysis of the hybridization pattern identifies the presence of the Ebola virus, and the presence of the particular strain of interest.

### Example 15

Sequences present in some human Y chromosomes but otherwise absent from the human genome and all known human SNPs, and not convertible into a perfect match for any non-Y human sequence or SNP-modified sequence by any combination of 1, 2 or 3 mismatches are identified, and 8-mer LNA probes to a subset of these sequences are synthesized and immobilized on individual beads of a bead-based hybridization assay. The assay is applied to pooled human female DNA samples, and a few nominally Y-specific probes are found to give false positive results. Results obtained from these probes are subsequently ignored. Nucleic acids isolated from female-subject forensic samples are assayed using this probe set, and Y-derived sequences are found, serving as evidence in criminal prosecution.

### Example 16

Probes complementary to sequences absent from the known genome sequence of a pregnant human female are synthesized and arrayed on a 3-dimensional gel pad. Amplified nucleic acids derived from a sample of the subject's blood are hybridized to the array, and the probes giving hybridization are compared with the archival data for similarly-amplified nucleic acids derived from a sample of the subject's blood taken before her pregnancy. The differences observed are used in conjunction with data from male subjects for the establishment of paternity.

### Example 17

Probes complementary to sequences absent from the known genome sequence of a pregnant human female are synthesized and arrayed on a 3-dimensional gel pad. Amplified nucleic acids derived from a sample of the subject's blood are hybridized to the array, and the probes giving hybridization are compared with the archival data for similarly-amplified nucleic acids derived from a sample of the subject's blood taken before her pregnancy. The differences observed are used for prenatal diagnosis of the status of the fetus.

### Example 18

Sequences absent from the human genome and all known human SNPs, and not convertible into a perfect match for any human DNA or RNA sequence or SNP-modified sequence by any combination of 1, 2 or 3 mismatches are identified, and probes to a subset of these sequences with predicted hybridization melting temperature in the range 62-65 degrees C., complementarity to known HIV-2 sequences, few-hundred base spacing, facing orientation, and limited propensity to hybridize to each other are synthesized and blended into a PCR mixture. Nucleic acids reverse transcribed from blood samples of patients showing immunodeficiency of unknown origin are added to the mixture and aliquots subjected to PCR under a range of conditions. Positive amplification results are diagnostic of HIV-2.

### Example 19

Sequences absent from the bovine genome and all known bovine SNPs, and not convertible into a perfect match for any bovine sequence or SNP-modified sequence by any combination of 1, 2 or 3 mismatches, and also not present in any sequence found by 700 megabases of shotgun sequencing of total DNA from bovine rumen contents are identified, and probes to a subset of these sequences with predicted complement hybridization melting temperature in the range 62-65 degrees C. are synthesized and immobilized on an array. Hybridization of rumen contents DNA to these arrays is used for routine monitoring of the health and nutritional status of cows, and for determining responses to changes in diet.

### Example 20

Sequences absent from the bovine genome and all known bovine SNPs, and not convertible into a perfect match for any bovine sequence or SNP-modified sequence by any combination of 1, 2 or 3 mismatches, and also not present in any sequence found in known human food pathogens are identified, and probes to a subset of these sequences with predicted complement hybridization melting temperature in the range 62-65 degrees C. are synthesized and immobilized on an array. Hybridization of nucleic acids derived from meat samples to these arrays is used for food safety assurance.

### Example 21

Sequences absent from all but one gene of the human genome and all known human SNPs, and not convertible into a perfect match for any other human sequence or SNP-modified sequence by any combination of 1 or 2 mismatches are identified. An array of DNA probes complementary to these sequences and 1-base modifications of them is used for resequencing of a large portion of the gene in multiple individuals.

### Example 22

Sequences absent from all but the mitochondrial portion of the human genome and all known human SNPs, and not convertible into a perfect match for any other human sequence or SNP-modified sequence by any combination of 1 or 2 mismatches are identified. An array of DNA probes complementary to these sequences and 1-base modifications of them is used for sequencing of a large portion of the mitochondrial genome in multiple individuals.

### Example 23

Sequences complementary to portions of the RNA genome of a virus and absent from the human genome and all known human SNPs, and not convertible into a perfect match for any other human sequence or SNP-modified sequence by any combination of 1 or 2 mismatches are identified. Several of these sequences are synthesized as DNA oligonucleotides fused to T7 RNA polymerase promoter sequences. These fused oligonucleotides are added to total nucleic acids isolated from patients suspected to be infected with the virus, with each oligonucleotide being added to the clinical sample in a separate reaction microwell. Treatment with reverse transcriptase followed by T7 RNA polymerase along with cy5-labeled nucleotides produces a fluorescent RNA product in 5 of 7 wells; this is interpreted as positive evidence that the patient is infected with the virus.

### Example 24

19-mer sequences absent from the human genome and all known human SNPs, and not convertible into a perfect match for any human sequence or SNP-modified sequence by any combination of 1, 2 or 3 mismatches are identified, and primers recognizing a subset of these sequences, with very limited propensity to hybridize to each other, is synthesized and blended into a PCR mixture. Nucleic acids isolated from atherosclerotic vessel biopsies from human patients are added, along with a primer directed to a highly-conserved region of eubacterial 16S ribosomal RNA and aliquots subjected to PCR under a range of conditions in a gradient thermocycler with 2 cycles of PCR under low stringency, followed by 30 cycles at higher stringency. Sequences from a previously unknown bacterium are amplified; sequencing produces enough 16S rRNA sequence for approximate classification of the organism.

### Example 25

Subsequences of each of the known genomic DNA sequences of C. jejuni are determined, and sequences common to nearly all strains of the organism and not found in the human DNA sequence or known SNPs are identified. The masses of DNA fragments of 8-25 nucleotides predicted to result from simultaneous digestion with multiple restriction endonucleases of DNA containing these subsequences are calculated. Nucleic acids isolated from human clinical samples are subjected to digestion with these restriction endonucleases and the masses of fragments determined by MALDI-TOF mass spectrometry. Matching of a predicted fragment mass is used to identify the presence of C. jejuni.

### Example 26

Subsequences of each of the known genomic DNA sequences of C. jejuni are determined, and sequences common to nearly all strains of the organism and not found in the human DNA sequence or known SNPs are identified. The masses of DNA fragments of 8-25 nucleotides predicted to result from simultaneous digestion with multiple restriction endonucleases of DNA containing these subsequences are calculated. Nucleic acids isolated from human clinical samples are subjected to digestion with these restriction endonucleases and the masses of fragments determined by MALDI-TOF mass spectrometry. Matching of a set of predicted fragment masses is used to identify the presence of C. jejuni.

### Example 27

Sequences absent from the human genome and all known human SNPs, and not convertible into a perfect match for any human sequence or SNP-modified sequence by any combination of 1, 2 or 3 mismatches are identified, and a subset of these sequences with predicted hybridization melting temperature in the range 62-65 degrees C. is immobilized in a 10,000-element array. A library of hybridization patterns on this array patterns produced by each of 50 pathogens of concern is developed in control experiments such that the terrorist agent can be unequivocally identified by matching with the pattern library.

### Example 28

Sequences absent from the human genome and all known human SNPs, and not convertible into a perfect match for any human sequence or SNP-modified sequence by any combination of 1, 2 or 3 mismatches are identified, and a subset of these sequences with predicted hybridization melting temperature in the range 65-70 degrees C. is immobilized in 8,000 elements of a 10,000-element array. The remaining 2,000 elements of the array are devoted to not-human-complimentary 16S rRNA signature sequences (Zhang, Z., Willson, R. C., and Fox, G. E. “Identification of Characteristic Oligonucleotides in the 16S Ribosomal RNA Sequence Dataset,” Bioinformatics, 18: 244-250 (2002)) chosen to be characteristic of various pathogenic subgroups. A library of hybridization patterns on this array patterns produced by each of 50 pathogens of concern is developed in control experiments such that a bioterrorism agent can in most cases be unequivocally identified by matching with the pattern library. However, a terrorist might seek to evade this highly sensitive system by transferring pathogenic genes to a closely related but normally non-pathogenic strain or by using a novel pathogenic strain. In either case, a pattern would be generated that would not be recognizably similar to any of the control patterns. In order to over come this problem, the phylogenetically characteristic signature probes are used such that when a pattern is produced which is not in the library, it is still possible to determine the genetic affinity of the pathogen being used. This is done by determining which of the phylogenetically-informative hybridization probes gave a positive result. Hence we might learn that the pathogen is a close relative of B. anthracis.

### Example 29

Subsequences of each of the known 16S rRNA sequences of B. anthracis are determined, and sequences common to nearly all strains of the bacterium and not found in the human sequence, other bacterial sequences, or known SNPs are identified. Locked nucleic acid probes to these sequences are synthesized and are immobilized on a collection of optically coded Luminex xMAP beads. Total RNA isolated from unknown bacterial samples are fragmented, labeled, and hybridized to the bead array. The bead array is analyzed, and some samples are found to be B. anthracis.

### Example 30

19-mer sequences absent from the human genome and all known human SNPs, and not convertible into a perfect match for any known human, microbial or viral sequence or SNP-modified sequence by any combination of 1, 2 or 3 mismatches are identified, and probes to a subset of these sequences with predicted hybridization melting temperature lying in a range spanning 5 degrees C. is synthesized in an array of 300,000 probes arranged in 50,000 spots with 6 types of probes in each spot. Nucleic acids isolated from blood samples from patients showing fevers of unknown origin are labeled and hybridized to the array, as are samples from control subjects. Spots absent in the control samples and often found in the fever-associated samples are found, and separate analysis of the probe sequences found in these spots identifies probes useful in detecting the causative agent of the fever.

### Example 31

A set of 19-mer probes not complementary to any sequence in the mosquito or human sequences or any 1- or 2-base modifications of these sequences, and complementary to the genome sequences of known mosquito-born viral pathogens is immobilized on an array. Nucleic acids isolated from blood-feeding mosquitoes are hybridized to the array and the results used to identify the pathogenic viruses carried by mosquitoes in a study area irrespective of whether or not the mosquito has recently ingested human blood.

### Example 32

A human/mosquito blind array is constructed that has a set of random 19-mer probes not complementary to any sequence in the mosquito or human sequences or any 1- or 2-base modifications of these sequences. The array has 40,000 spots each containing 10 random sequences. Nucleic acids isolated from blood-feeding mosquitoes are hybridized to the array and the results used to identify the pathogenic viruses carried by mosquitoes in a study area.

### Example 33

A human/mosquito blind array is constructed that has a set of random 19-mer probes not complementary to any sequence in the mosquito or human sequences or any 1- or 2-base modifications of these sequences. The array has 40,000 spots each containing 10 random sequences. Characteristic hybridization patterns for a variety of known viruses are cataloged, and nucleic acids isolated from blood-feeding mosquitoes are hybridized to the array and the results used to identify the pathogenic viruses carried by mosquitoes in a study area.

### Example 34

Subsequences of each of the known sequences of Ebola virus are determined, and sequences common to nearly all strains of the virus and not found in the human sequence or known SNPs are identified. 19-mer sequences absent from the human genome and from all but one known Ebola sequence are also identified. Among these sequences, the subset not convertible into a perfect match for any human sequence or SNP-modified sequence by any combination of 1, 2 or 3 mismatches are selected and are immobilized on a microscope slide, and hybridized with labeled human clinical samples. Positive hybridization to both types of probes identifies the presence of the Ebola virus, and the presence of the particular strain of interest.

### Example 35

Sequences absent from the human genome and all known human SNPs, and not convertible into a perfect match for any human sequence or SNP-modified sequence by any combination of 1, 2 or 3 mismatches are identified, and a subset of these sequences with predicted hybridization melting temperature in the range 62-65 degrees C. is synthesized and blended in to a PCR mixture. Nucleic acids isolated from liver biopsies from patients showing hepatitis of unknown origin are added to the mixture and aliquots subjected to PCR under a range of conditions. Sequences from a previously unknown virus distantly related to the Hepatitis C virus are amplified.

### Example 36

Sequences absent from the human genome and all known human SNPs, and not convertible into a perfect match for any human sequence, SNP-modified sequence or the genes encoding production of a therapeutic antibody by any combination of 1, 2 or 3 mismatches are identified, and a subset of these sequences with predicted hybridization melting temperature in the range 62-65 degrees C. is synthesized and blended in to a PCR mixture. Nucleic acids isolated from human cell line cultures used in production of the therapeutic antibody are added to the mixture and aliquots subjected to PCR under a range of conditions. The absence of amplification (and successful amplification of spiked positive control mixtures) is used as a quality control measure before release of the production batch.

### Example 37

Company X is using a novel strain of a bacterium to process an organic waste in an above ground bioreactor. Nearby, several children become extremely sick from a bacterial infection that can be traced to apparent contamination of a private swimming pool. Unfortunately, the problem bacterium can not be cultivated or isolated. A highly reputable private laboratory was hired to investigate further. Using PCR probes targeting 16S rRNA they showed that organisms with the same 16S rRNA sequence as the strain used in the company's bioreactor are present in soil samples taken near the swimming pool. The parents are now suing the company for large sums of money. In order to determine if it is really the company strain that is or is not present in the soil samples the genome of the company strain is sequenced. Next, sequences of length N which are blind to all known bacteria are identified. A hybridization array containing 1000 randomly chosen bacterial blind sequences of length N is constructed. If the company strain is present, essentially every probe will give a positive result. However, because the genomes change significantly for even closely related strains, if the problem organism is not the company strain a large number of probes will not give a signal. Key controls will be soil samples known to contain the company strain, soil known to contain the company DNA and a third sample containing a noncompany strain of the problem organism.

### Example 38

### Universal Identification System

It is of concern that any of a number of bacterial pathogens might be used as bioterrorist agents and therefore a universal identification system is desirable. We therefore use the methodology above to develop an array of 1000 random human blind N-mers of similar hybridization properties. For bacterial size genomes when these oligomers hybridize they produce a unique pattern that changes very rapidly so that even closely related strains can be distinguished. For example, all three E. coli strains whose genome has been sequenced can be readily distinguished. A library of patterns produced by each pathogen of concern (e.g. NIAID A, B and C lists) is developed in control experiments such that matching with the pattern library can unequivocally identify the terrorist agent. However, a terrorist might seek to evade this highly sensitive system by transferring pathogenic genes to a closely related but normally non-pathogenic strain or by using a novel pathogenic strain. In either case, a pattern would be generated that would not be recognizably similar to any of the control patterns. In order to overcome this problem, a large number of human blind16S rRNA signature sequences are calculated. Human-blind signature sequences for various pathogenic subgroups with appropriate hybridization properties are then incorporated into the universal array. This adds a phylogenic component to the array such that when a pattern is produced which is not in the library; it is still possible to determine the genetic affinity of the pathogen being used. Determining which of the phylogenetically informative hybridization probes gave a positive result does this. Hence we might learn that the pathogen is a close relative of B. anthracis.

### Example 39

Environmental issues necessitate a better understanding of microbial ecosystems. In order to understand a complex ecosystem one needs to know what the major microbial components of the ecosystem are. One can identify many of the major components with culture studies or by sequencing 16S rDNA fragments produced by PCR. However, it is in either case difficult to know the extent to which the population diversity has actually been determined. This is addressed by constructing an array of probes which are blind to the genomes of all known bacteria in the ecosystem of interest. If a unique genome of n megabases is present it will be expected to react positively to a predictable number of random all bacterial blind probes. The number of probes that do react will allow statistical estimation of the number major ecosystem components that remain unidentified.

### Example 40

Subsequences of each of the known sequences of the yellow fever virus are determined, Sequences common to nearly all strains of the virus and not found in the human sequence, known human SNPs, or the known genomic sequence of Aedes aegypti (the yellow fever mosquito) are identified. Molecular beacon fluorescent probes complementary to 100 all-strain- and single-strain-characteristic sequences are synthesized. All the molecular beacons will emit fluorescent light at the same wavelength upon hybridization with their target sequence. This allows amplification of the total signal while accounting for the presence of essentially any yellow fever strain in the mosquitoes being tested. In order to conduct a test, homogenized mosquitoes will simply be added to a premixed set of molecular beacon probes and fluorescence monitored. The test will be readily used in remote areas where sophisticated instrumentation is not readily available. It will provide a positive result for any yellow fever strain and will still work even if the mosquitoes have recently ingested human blood.

### Example 41

Environmental issues necessitate a better understanding of microbial ecosystems. In order to understand a complex ecosystem one needs to know what the major microbial components of the ecosystem are. One can identify many of the major components with culture studies or by sequencing 16S rDNA fragments produced by PCR. However, it is difficult to know the extent to which the population diversity has actually been determined. This is addressed by constructing an array of probes which are blind to the genomes of all known bacteria in the ecosystem of interest. If a unique genome of n megabases is present it will be expected to react positively to a predictable number of random all bacterial blind probes. The number of probes that do react will allow statistical estimation of the number of major ecosystem components that remain unidentified.

### Example 42

Sequences differing in one position from the most-closely matching DNA of the mitochondrial portion of the human genome and all known human SNPs, and not convertible into a perfect match for any other human sequence or SNP-modified sequence by any combination of 1 or 2 or 3 mismatches are identified. An array of DNA probes complementary to these sequences and 1-base modifications of them is used for sequencing of a large portion of the mitochondrial genome in multiple tissue samples and biopsy specimens from a single individual to monitor the appearance of mutations.

### Example 43

Sequences predicted to be present in virus-encoded mRNA based on cDNA databases and genome annotation, but absent from the mouse genomic DNA sequence and all known mouse SNPs and not convertible into a perfect match for any mouse genomic DNA sequence or SNP-modified sequence by any combination of 1 or 2 mismatches are identified. Probes to a subset of these sequences with predicted DNA probe/mRNA hybridization melting temperature in the range 63-70 degrees C. are synthesized as DNA probes and spotted onto a microscope slide, and hybridized with labeled mRNA amplified from virus-infected mouse samples. The pattern of gene expression inferred from the hybridization results is used to study the course of viral infection and the effects of candidate therapeutics.

### Example 44

Sequences absent from the human genome and all known human SNPs, and not convertible into a perfect match for any human sequence or SNP-modified sequence by any combination of 1, 2 or 3 mismatches, and also not present in any sequence found by 300 megabases of shotgun sequencing of total DNA from human oral biofilm contents are identified, among this set sequences specific to a particular organism thought to act as a competitive inhibitor of human dental caries formation are identified. Probes to a subset of these sequences with predicted complement hybridization melting temperature in the range 62-65 degrees C. are synthesized and immobilized on an array. After administration of the organism, hybridization of oral rinse-derived DNA to these arrays is used for monitoring of the survival and spread of the caries-prevention organism in the mouth.

### Example 45

Sequences not found in 900 megabases of random sequencing of DNA isolated from a hazardous-waste spillage site and not convertible into a perfect match for any such sequence by any combination of 1, 2 or 3 mismatches are identified, and among this set sequences specific to a particular plasmid known to carry genes for waste degradation are identified. Probes to a subset of these sequences with predicted complement hybridization melting temperature in the range 60-70 degrees C. are synthesized and immobilized on an array. Hybridization of soil-derived DNA to these arrays is used for monitoring of the survival and spread of the plasmid in this ecosystem.

### Example 46

Subsequences of each of the known sequences of Ebola virus are determined, and 18-mer sequences common to nearly all strains of the virus and not found in the human sequence or known SNPs are identified. 22-mer DNA probes complementary to these 18-mer sequences, but extended by 2 bases (equimolar mixtures of the 4 natural DNA nucleotides) at the 3′ end, and by two nucleotides (inosines) at the 5′ end, are immobilized on a microscope slide, and hybridized with labeled human clinical samples. Positive hybridization to these probes identifies the presence of the Ebola virus.

### Specific Objects

Other objects of the inventions comprise the determination and identification of other microorganisms, valuable or deleterious, which are comprised in a host, or the confirmation of their absence.

### Modifications

Specific compositions, methods, or embodiments discussed are intended to be only illustrative of the invention disclosed by this specification. Variations on these compositions, methods, or embodiments are readily apparent to a person of skill in the art based upon the teachings of this specification and are therefore intended to be included as part of the inventions disclosed herein. For example, the growth of genome and SNP databases will readily allow the development of additional probe/primer sequences for detection of newly-sequenced organisms, or “blind” to these organisms, or blind to additional genetic variants by the methods disclosed here. Also it will be obvious to skilled persons that longer N-mers than those recited herein may be used to great advantage in specific applications of the invention.

There are many variations to the invention; including its processes, processes of using sequences listed in the Contents and in the CDs, the sequences (subsequences) themselves, the related compositions and probes, the arrays, and the algorithms set forth in the Specification.

For example, the invention comprises a process for identifying whether a specific target organism (including viruses) is present in a sample, by the process comprising in combination: a. identifying at least one nucleic acid sequence (n-mer) characteristic of the target organism; b. identifying at least one nucleic acid which may be present in the sample and is characteristic of one or more other (nontarget) organisms which may be present in the sample; c. identifying nucleic acid sequence(s) of the organism or virus differing in at least N positions (N mismatches, where N is 1-10) from the nucleic acid sequence(s) of one or more other (non target) organisms which may be present in the sample; and d. determining the presence in the sample of one or more sequence(s) from step a which do not meet the criteria of step b and do meet those of step c, whereby the presence of such sequences indicates the presence of the target organism.

Preferred are such processes in which at least some of the nucleic acid sequences of steps a, b, and c are identified be selecting from a database; or in which at least some of the nucleic acid sequences of steps a, b, and c are identified by selecting from a database listed in the attached CDs; or in which at least some of the nucleic acid sequences of step d are not assayed to be present, and the target organism is determined to be present by the application of a mathematical decision formula; or in which at least some of the nucleic acid sequences of step b are assayed to be present, and the target organism is determined to be present by the application of a mathematical decision formula; or in which at least some of the nucleic acid sequences of step b are assayed to be present, and at least some of the nucleic acid sequences of step d are not assayed to be present, and the target organism is determined to be present by the application of a mathematical decision formula. Especially preferred are the use of 5, 10, 100 or 1000 or more sequences (subsequences).

The invention also comprises processes for identifying whether an organism or virus is present in a sample, the processes comprising in combination: a. identifying nucleic acid signature(s) of the organism or virus, b. identifying at least 100 nucleic acid signature(s) of one or more other organisms and/or viruses which may be present in the sample, c. identifying nucleic acid signature(s) of the organism or virus differing in at least N positions (N MM) from nucleic acid signature(s) of one or more other organisms and/or viruses which may be present in the sample, where N is selected to give a desired improbability of mistaken detection of the target organism, d. determining the presence in the sample of sequence(s) from step a which do not meet the criteria of step b and do meet those of step c, whereby the presence of such sequences indicates the presence of the target organism. Particularly preferred are such processes wherein the organism of steps b or c is an animal host, whereby there is created a set of host-blind sequences; or wherein N is about 1 to 5; or wherein said host-blind sequences comprise n-mers, wherein n is from 5 to 25.

Also the invention comprises processes using a specific human-blind sequence from [File] SEQ ID NO 1 to 17 to identify or detect the associated organism in the presence of human RNA or DNA; and each specific human-blind sequence 11- to 18-mer human-blind sequence from SEQ ID No 1-17; and each specific human-blind sequence 18-mer shown in folder microbial SEQ ID NO 1 [CD1]; and each specific host-blind sequence 11- to 13-mer shown in folder human mismatches SEQ ID NO 2 [CD1]; and each specific human-blind sequence 14-mer shown in folder human mismatches SEQ ID NO 3 [CD2]; and each specific human-blind sequence 18-mer shown in folder virus 18 mers 2MM SEQ ID NO 4 [CD3]; and each specific human-blind sequence 17-mer shown in folder virus 17 mers 2MM SEQ ID NO 6 [CD3]; and each specific human-blind sequence 17-mer shown in folder virus 17-mers 1MM SEQ ID NO 7 [CD3] and each specific human-blind sequence 17-mer shown in folder selected microbe 17 mers SEQ ID NO 8 [CD3]; and each specific human-blind sequence 18-mer shown in folder selected microbe 18 mers SEQ ID NO 9 [CD3]; each specific human-blind sequence 18-mer shown in folder Microbial 17-mers 2MM SEQ ID NO 10 [CD4]; each specific human-blind sequence 17-mer shown in folder Microbial 17-mers 1MM SEQ ID NO 11 [CD4]; and each specific human-blind sequence 17-mer shown in folder Microbial 17 mers 1MM SEQ ID NO 12 [CD5]; and each specific human-blind sequence 17-mer shown in folder Microbial 17-mers 1MM SEQ ID NO 13 [CD6]; and each specific human-blind sequence 17-mer shown in folder Microchip 17-mers 1MM SEQ ID NO 14 [CD6]; and each specific human-blind sequence shown in CD7, Folder nmerseqsWNV, SEQ ID NO 15 [CD7] [contains 465 West Nile virus], Folder nmerseqsDV, SEQ ID NO 16 [CD7] [contains 1815 Dengue virus], and/or Folder nmerseqsAllFlavi, SEQ ID NO 17 [CD7] [contains 765 Flavivirus].

The invention also comprises collections of at least 10 sequences from SEQ ID NO 1 to 17; and preferred, collections of probe sequences comprising at least 5, more preferably 10, and most preferably 100 or more of the sequences associated with a single organism in SEQ ID NO 1 to 17 [CDs 1-7]; and collections of probe sequences comprising at least 5% (more preferably 25 and most preferably 90%) of the sequences associated with a single organism in SEQ ID NO 1 to 17.

Still further, the invention comprises compositions comprising target organism nucleic acids (or nucleotides) hybridized with probe sequences selected from SEQ ID NO 1 to 17; preferably compositions comprising at least 20 probe sequences selected from SEQ ID NO 1 to 17 and more preferably located in a different position from each other; more preferably with detectable labels and/or polymerase.

Valuable embodiments of the invention comprise other chemical forms of probes besides ordinary DNA, and also cover longer and shorter sequences derived from the ones in the enclosed CDs. For example, composition comprising at least 10 DNA, RNA, LNA, PNA, modified backbone DNA, or modified base probe sequences selected from SEQ ID NO 1 to 17, and a detectable label and composition comprising at least 10 probe sequences selected from the sequences, extensions of the sequences, and truncated derivatives of length at least 12, of the sequences SEQ ID NO 1 to 17.

The invention offers processes for monitoring gene expression of a target organism or virus in a sample, the process comprising in combination: a. identifying nucleic acid signature(s) of the organism or virus, b identifying at least 100 nucleic acid signature(s) of one or more nontarget organisms and/or viruses which may be present in the sample, c. identifying nucleic acid signature(s) of the organism or virus differing in at least N positions from nucleic acid signature(s) of one or more nontarget organisms and/or viruses which may be present in the sample, where N is selected to give a desired certainty of non-hybridization to nucleic acids of the nontarget organism; d. determining the concentration in the sample of sequence(s) from step a which do not meet the criteria of step b and do meet those of step c, whereby the presence of such sequences indicates gene expression of the target organism.

Also encompassed by the invention are processes for detecting the presence of a virus or other non-human organism in human-derived samples, comprising in combinations: a. preparing an array of oligonucleotide probe sequences selected not to hybridize to any known human sequence or any sequence derivable from any known human sequence by any combination of 3 or fewer changes, b. hybridizing labeled nucleic acids isolated from a human blood sample to the array, c. observing the resulting hybridization pattern. And also processes for detecting the presence of a virus or other non-human host organism in human-derived samples, comprising in combination: a. preparing a mixture of 14- to 20-mer oligonucleotide primer sequences including sequences selected not to hybridize to any known human sequence or any sequence derivable from any known human sequence by any combination of 3 or fewer changes, but within five sequence changes (mismatches) of sequences complimentary to known target sequences, with the primer sequences selected not to give amplification with flavivirus-free human nucleic acid samples, b. Performing RT-PCR with these primers and nucleic acids isolated from a human blood sample, c. observing amplification with blood samples from humans infected with the target virus. Especially preferred in any of the above is the process wherein the target is flavivirus.

For scanning, the invention can employ processes for identifying whether any other organism/guest/parasite/ is present in a biologic host, the process comprising in combination: a. scanning for non-host signatures, and b. scanning for N-error-removed non-host signatures; where N is selected to give the desired statistical certainty of the presence or absence of any parasite in the host and preferably wherein N=1 to 10, more preferably 2-8 and most preferably 3-5.

As to sequences, the invention comprises each specific human-blind motif [n-mer/signature/sequence/subsequence shown in CD1 to CD7 preferably those of n=7 to 25-mers and also each correlation of n-mer vs. genome (sequence/signature/n-mer/organism shown in CD1 to CD7 [SEQ ID 1-17].

And also arrays [“chips”] of at least 10 [preferably 5 and more preferably 2 of the signatures shown in CD1 to CD7.

The mathematical aspects of the invention include each algorithm shown in this application, especially those for comparing yes/no testing to identify an ultraspecific microorganism.

Specific fragments can include an isolated polynucleotide fragment comprising a nucleic acid sequence selected from the group consisting of: SEQ ID NOs 1 to 17 and an isolated polynucleotide complementary to the polynucleotide of any of the claims; preferably those isolated polynucleotides which polynucleotide comprise a heterologous nucleic acid sequence; more preferably an isolated polynucleotide of claim 3, where the heterologous nucleic acid sequence encodes a heterologous polypeptide. Also valuable are uses of the invention for making a recombinant vector comprising inserting the isolated polynucleotide of claim 1 into a vector.

Reference to documents made in the specification is intended to result in such patents or literature being expressly incorporated herein by reference. Four unpublished drafts and a list of references cited above are enclosed and incorporated by reference. Larger print copies are available on request to the Attorney.

SEQUENCE LISTING

The patent application contains a lengthy “Sequence Listing” section. A copy of the “Sequence Listing” is available in electronic form from the USPTO web site https://bulkdata.uspto.gov/data2/lengthysequencelisting/2017/. An electronic copy of the “Sequence Listing” will also be available from the USPTO upon request and payment of the fee set forth in 37 CFR 1.19(b)(3).

What is claimed is:

1. A process for identifying whether a specific target organism (including viruses) is present in a sample, the process comprising in combination:
a. identifying at least one nucleic acid sequence (n-mer) characteristic of the target organism;
b. identifying at least one nucleic acid which may be present in the sample and is characteristic of one or more other (nontarget) organisms which may be present in the sample;
c. identifying nucleic acid sequence(s) of the organism or virus differing in at least N positions (N mismatches, where N is 1-10) from the nucleic acid sequence(s) of one or more other (non target) organisms which may be present in the sample; and
d. determining the presence in the sample of one or more sequence(s) from step a which do not meet the criteria of step b and do meet those of step c, whereby the presence of such sequences indicates the presence of the target organism.

2. A process according to claim 1 in which at least some of the nucleic acid sequences of steps a, b, and c are identified by selecting from a database listed in the attached CDs 1-7.

3. A process according to claim 1 in which at least some of the nucleic acid sequences of step d are not identified to be present, and the target organism is determined to be present by the application of a mathematical decision formula.

4. A process according to claim 1 in which at least some of the nucleic acid sequences of step b are assayed to be present, and the target organism is determined to be present by the application of a mathematical decision formula.

5. A process according to claim 1 in which at least some of the nucleic acid sequences of step b are identified to be present, and at least some of the nucleic acid sequences of step d are not identified to be present, and the target organism is determined to be present by the application of a mathematical decision formula.

6. A process according to claim 1 for identifying whether an organism or virus is present in a sample, the process comprising in combination:
a. identifying nucleic acid signature(s) of the organism or virus,
b. identifying at least 100 nucleic acid signature(s) of one or more other organisms and/or viruses which may be present in the sample,
c. identifying nucleic acid signature(s) of the organism or virus differing in at least N positions (N MM mismatches) from the nucleic acid signature(s) of one or more other organisms and/or viruses which may be present in the sample, where N is selected to give a desired improbability of mistaken detection of the target organism.
d. determining the presence in the sample of sequence(s) from step a which do not meet the criteria of step b and do meet those of step c, whereby the presence of such sequences indicates the presence of the target organism.

7. A process according to claim 1 wherein the organism of steps b or c is an animal or plant host, whereby there is created a set of host-blind sequences.

8. A process according to claim 1 wherein N is about 1 to 5.

9. A process according to claim 7 wherein said host-blind sequences comprise n-mers, wherein n is from 5 to 25.

10. The process of using a specific human-blind sequence from SEQ ID NO 1 to 17 to identify or detect the associated organism in the presence of human RNA or DNA.

11. Each specific human-blind sequence 11- to 18-mer human-blind sequence from SEQ ID No 1-17.

12. A collection of at least 10 sequences according to claim 11 from SEQ ID NO 1 to 17.

13. A collection of probe sequences according to claim 13 comprising at least 5 sequences associated with a single organism in SEQ ID NO 1 to 17 or derivatives of these sequences or equivalent.

14. A composition according to claim 13 comprising target organism nucleic acids hybridized with probe sequences selected from SEQ ID NO 1 to 17.

15. A composition according to claim 13 comprising at least 10 DNA, RNA, LNA, PNA, modified backbone DNA, or modified base probe sequences selected from SEQ ID NO 1 to 14, and a detectable label.

16. A composition according to claim 13 comprising at least 10 DNA, RNA, LNA, PNA, modified backbone DNA, or modified base probe sequences selected from the sequences, extensions of the sequences, and truncated derivatives of length at least 12, of the sequences SEQ ID NO 1 to 17.

17. A process according to claim 1 for detecting the presence of a virus or other non-human organism in human-derived samples, comprising in combinations:
a. preparing an array of oligonucleotide probe sequences selected not to hybridize to any known human sequence or any sequence derivable from any known human sequence by any combination of 3 or fewer changes,
b. hybridizing labeled nucleic acids isolated from a human blood sample to the array,
c. examining the resulting hybridization pattern for patterns characteristic of the virus or other non-human organism.

18. An array of at least 5 of the signatures of claim 13 shown in CD1 to 17.

19. Each algorithm shown in the specification for identifying an ultraspecific microorganism.

20. A process according to claim 1 comprising the use of an algorithm shown in the specification for identifying a microorganism.
